(19) United States
(12) Patent Application Publication
(10) Pub. No.: US 2026/0207786 A1
(43) Pub. Date: Jul. 23, 2026

(54) METHODS AND COMPOSITIONS FOR IMPROVED DELIVERY VIA ULTRASOUND

(71) Applicant: SonoThera, Inc., South San Francisco, CA (US)

(72) Inventors: Ivan KRIVEGA, Burlingame, CA (US); Margarita KRIVEGA, South San Francisco, CA (US); Steven B. FEINSTEIN, Highland Park, IL (US); Kenneth GREENBERG, Berkeley, CA (US)

(21) Appl. No.: 19/391,999

(22) Filed: Nov. 17, 2025

Related U.S. Application Data
(60) Provisional application No. 63/722,046, filed on Nov. 18, 2024.
(60) Provisional application No. 63/722,045, filed on Nov. 18, 2024.
(60) Provisional application No. 63/774,082, filed on Mar. 18, 2025.
(60) Provisional application No. 63/805,758, filed on May 14, 2025.
(60) Provisional application No. 63/894,540, filed on Oct. 6, 2025.

(51) Int. Cl. A61K 48/00; A61K 38/17; A61K 41/00

(52) U.S. Cl. CPC A61K 48/0058; A61K 38/1709; A61K 41/0047

(57) ABSTRACT

Provided are methods and compositions for expressing a functional dystrophin protein or a functional fragment thereof in a subject. The method can include administering a nucleic acid encoding the dystrophin protein to the subject, administering a sonoactive agent to the subject, and applying ultrasound energy the cell. Also provided are use of a nucleic acid encoding a functional dystrophin protein and a sonoactive agent in a method of treating a subject having a dystrophinopathy disorder requiring a dystrophin gene therapy or a dystrophin protein replacement therapy.

### CROSS REFERENCE TO RELATED APPLICATIONS

This application claims the benefit of U.S. Provisional Patent Application No. 63/722,045 filed Nov. 18, 2024, U.S. Provisional Patent Application No. 63/774,082 filed Mar. 18, 2025, U.S. Provisional Patent Application No. 63/805,758 filed May 14, 2025, U.S. Provisional Patent Application No. 63/894,540 filed Oct. 6, 2025, and U.S. Provisional Patent Application No. 63/722,046, filed Nov. 18, 2024, each of which is incorporated herein by reference in its entirety and for all purposes.

### INCORPORATION BY REFERENCE OF SEQUENCE LISTING

The present application is being filed along with a Sequence Listing in electronic format. The Sequence Listing is provided as a file entitled SequenceListing62668742201.XML, created Nov. 17, 2025, which is 453,266 bytes in size. The information in the electronic format of the Sequence Listing is incorporated by reference in its entirety.

### BACKGROUND

Gene therapy, in which a functional copy of a gene is transfected into a cell, has been proposed as a possible method of treating genetic diseases. One proposed method of delivering a gene therapy to a subject is delivery of therapeutic agents using ultrasound and microbubbles, also referred to as sonoporation. However, prior art methods of gene therapy using ultrasound or sonoporation suffer from significant shortcomings such as low transfection rates, and insufficient gene expression, which have prevented the clinical development and commercialization of these methodologies. There remains a need in the art for more effective gene therapy techniques that can transfect a gene to a cell in an organ or a tissue in a subject in a safe, effective, and durable manner.

### SUMMARY

Dystrophinopathy disorders such as Duchenne muscular dystrophy (DMD), Becker muscular dystrophy (BMD), and DMD-associated dilated cardiomyopathy (DCM) are genetic disorders associated with disfunction of the dystrophin gene in mammalian subjects, and are among the most devastating genetic disorders of our time. Lacking a safe and effective treatment, affected subjects generally begin to experience symptoms of progressive muscle fiber degeneration and weakness in mid-childhood to teenage years, with most DMD patients having a life expectancy of only 18 to 25 years. While various gene therapy approaches attempt to a treat dystrophinopathy disorders within the limitations of current technology, such as with delivery of dystrophin truncates or dystrophin fusion proteins, there are no therapies which improve the functional outcome of affected patients by delivering a gene therapy which results in the production of a functional copy of the dystrophin protein.

Aspects disclosed herein provide a method of expressing a nucleic acid encoding a dystrophin protein in a cell of a subject, comprising: administering the nucleic acid encoding the dystrophin protein to the subject, administering a sonoactive agent to the subject, and applying ultrasound energy the cell. Aspects disclosed herein provide a method of treating a dystrophinopathy disorder in a subject in need thereof comprising administering to the subject a nucleic acid encoding a functional dystrophin protein, delivering the nucleic acid to a myocyte in a muscle tissue of the subject along a length of a muscle fiber in the muscle tissue and/or about the sarcolemma of a muscle fiber in the muscle tissue, thereby treating the dystrophinopathy disorder. Aspects disclosed herein provide a method of expressing dystrophin in a subject comprising: inducing uptake by a cell of an expression vector encoding a functional dystrophin protein or a functional fragment thereof. Aspects disclosed herein provide a method of expressing dystrophin in a subject comprising: inducing uptake by a cell in a population of cells of an expression vector encoding a functional dystrophin protein or a functional fragment thereof, in which at least 0.25% of cells in the population of cells uptake the expression vector. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject in an amount exceeding 0.5% of a dystrophin level in a wild type subject not having the dystrophinopathy disorder, thereby treating the dystrophinopathy disorder. Aspects disclosed herein provide a use of a nucleic acid encoding a functional dystrophin protein and a sonoactive agent in a method of treating a subject having a dystrophinopathy disorder requiring a dystrophin gene therapy or a dystrophin protein replacement therapy. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject about the cellular membrane of the myocyte, throughout the cytoplasm of the myocyte, or both about the about the cellular membrane and throughout the cytoplasm of the myocyte, thereby treating the dystrophinopathy disorder. Aspects disclosed herein provide a method of expressing dystrophin in a subject comprising: inducing uptake by a cell in a diaphragm of a subject of an expression vector encoding a functional dystrophin protein or a functional fragment thereof. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not conjugated to an antibody. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not associated with cationic peptide moieties. Aspects disclosed herein provide a method of treating a dystrophinopathy disorder in a subject in need thereof comprising administering to the subject a nucleic acid encoding a functional dystrophin protein, transfecting the nucleic acid to a muscle tissue of the subject, wherein at least 5% of muscle fibers in the muscle tissue of the subject contain the nucleic acid encoding the functional dystrophin protein. Aspects disclosed herein provide a method of delivering a nucleic acid encoding a functional dystrophin protein in a muscle cell in a muscle tissue of a subject, comprising: administering the nucleic acid encoding the dystrophin protein to the subject wherein a coding sequence of the nucleic acid encoding the dystrophin protein is at least 10000 nucleotides; administering a sonoactive agent to the subject; and applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue thereby disrupting the sonoactive agent and delivering the nucleic acid encoding the dystrophin protein to at least 5% of muscle fibers in the muscle tissue.

In some embodiments, the cells comprise a myocyte, and wherein the functional dystrophin protein or a functional fragment thereof is expressed in the myocyte in an amount exceeding 0.5% of a dystrophin level in a wild type subject. In some embodiments, the cells comprise a myocyte, and wherein the functional dystrophin protein or a functional fragment thereof is expressed (i) about the cellular membrane of the myocyte, (ii) throughout the cytoplasm of the myocyte, or (iii) both about the cellular membrane and throughout the cytoplasm of the myocyte. In some embodiments, the functional dystrophin protein positioned about the sarcolemma is located along a surface of the sarcolemma along a length of the sarcolemma in a direction parallel to the muscle fiber encapsulated within the sarcolemma. In some embodiments, the functional dystrophin protein is expressed in the myocyte in the muscle tissue. In some embodiments, the functional dystrophin protein is expressed along the length of the muscle fiber in the muscle tissue. In some embodiments, the functional dystrophin protein is about the sarcolemma of the muscle fiber in the muscle tissue. In some embodiments, the functional dystrophin protein transfers tension along the length of the muscle fiber or improves a maximum tension loading capacity of the muscle tissue. In some embodiments, at least 1, 3, 5, 10, 15, 20, 25, or 28% of the muscle fibers in the treated muscle tissue comprise the nucleic acid encoding the functional dystrophin protein. In some embodiments, the cell is a myocyte, wherein the dystrophin protein or a functional fragment thereof is expressed in the myocyte in an amount exceeding 0.5% of a dystrophin level in a wild type subject. In some embodiments, the method induces uptake of the nucleic acid into at least 0.25% of a population of cells comprising the cell. In some embodiments, the population of cells is in a cardiac tissue or a heart of the subject, a skeletal muscle tissue or a skeletal muscle of the subject, or a diaphragm tissue or a diaphragm of the subject. In some embodiments, the cell is a multinucleated cell, a myocyte or a muscle cell. In some embodiments, the myocyte or the muscle cell comprises a skeletal muscle myocyte, a cardiomyocyte, or a smooth muscle cell. In some embodiments, the myocyte or the muscle cell is located in a cardiac tissue or a heart of the subject. In some embodiments, dystrophin is expressed in the cardiac tissue or the heart in an amount of at least 0.3, 0.5, 0.75, 1, 2, 3, 4, 5, 6, 7, 8, 9, or 10% of a dystrophin level in human heart whole tissue lysate as measured by western blot assay. In some embodiments, the myocyte or the muscle cell is located in a skeletal muscle tissue or a skeletal muscle of the subject. In some embodiments, the dystrophin is expressed in the muscle tissue or the skeletal muscle in an amount of at least 0.3, 0.5, 0.75, 1, 2, 3, 4, 5% of a dystrophin level in human skeletal muscle whole tissue lysate when measured by western blot assay. In some embodiments, the myocyte or the muscle cell is located in a diaphragm tissue or a diaphragm of the subject. In some embodiments, the dystrophin is expressed in the diaphragm tissue or the diaphragm in an amount of at least 0.3, 0.5, 0.75, 1, 2, 3, 4, 5, 6, 7, 8, 9, or 10% of a dystrophin level in human diaphragm whole tissue lysate when measured by western blot assay. In some embodiments, the dystrophin is expressed in the cardiac tissue, the muscle tissue, or the diaphragm tissue, throughout a treated organ comprising the cardiac tissue, the muscle tissue, or the diaphragm tissue. In some embodiments, the dystrophin is expressed at detectable levels throughout the treated organ in two or more samples taken from opposite ends of the treated organ. In some embodiments, the nucleic acid or the expression vector is an unencapsulated nucleic acid. In some embodiments, the nucleic acid encodes a full length dystrophin protein. In some embodiments, a coding sequence of the nucleic acid encoding the full length dystrophin protein is at least 5000, 6000, 7000, 8000, 9000, 10000, or 11000 nucleotides. In some embodiments, a length the functional dystrophin protein is at least 1750, 2000, 2250, 2500, 2750, 3000, or 3250 amino acids. In some embodiments, the myocyte is a skeletal muscle myocyte and wherein dystrophin is expressed in the myocyte at a level of at least 1, 2, 3, 4, 5, 10, 20, 30, 40, 50, 60, 70, 80, 90, 100, 110, or 120% of a normal dystrophin level in a subject not having a dystrophinopathy disorder. A normal dystrophin level in skeletal muscle tissue generally refers to a dystrophin level in human skeletal muscle whole tissue lysate. In some embodiments, a coding sequence of the functional dystrophin protein is at least 5000, 6000, 7000, 8000, 9000, 10000, or 11000 nucleotides. In some embodiments, the method further includes administering a sonoactive agent to the subject, and applying ultrasound energy a cell of the subject. In some embodiments, a length the functional dystrophin protein is at least 1750, 2000, 2250, 2500, 2750, 3000, or 3250 amino acids. In some embodiments, a coding sequence of the functional dystrophin protein is at least 5000 nucleotides. In some embodiments, the nucleic acid encoding the functional dystrophin protein and the sonoactive agent are configured to induce expression of the functional dystrophin protein in a cell of the subject upon administration of ultrasound energy to the cell. In some embodiments, a coding sequence of the dystrophin protein is at least 6000, 7000, 8000, 9000, 10000, or 11000 nucleotides. In some embodiments, the coding sequence of the dystrophin protein encodes a functional dystrophin protein. In some embodiments, the dystrophin protein is at least 1750, 2000, 2250, 2500, 2750, 3000, or 3250 amino acids in length. In some embodiments, the coding sequence of the functional dystrophin protein encodes a full length dystrophin protein. In some embodiments, the functional dystrophin protein is at least 1750, 2000, 2250, 2500, 2750, 3000, or 3250 amino acids in length. In some embodiments, the nucleic acid encoding the dystrophin protein further comprises a promoter sequence. In some embodiments, the promoter sequence comprises a sequence of any one of SEQ ID NO: 4-5, or 18. In some embodiments, the nucleic acid encoding the dystrophin protein further comprises a post transcriptional regulatory element. In some embodiments, the post transcriptional regulatory element comprises the sequence of any one of SEQ ID NO: 8-10. In some embodiments, the nucleic acid encoding the dystrophin protein further comprises a nuclear targeting sequence. In some embodiments, the nuclear targeting sequence comprise a sequence of SEQ ID NO: 11. In some embodiments, the nucleic acid encoding the dystrophin protein further comprises a post transcriptional regulatory element and a nuclear targeting sequence. In some embodiments, the post transcriptional regulatory element and the nuclear targeting sequence comprise a sequence of SEQ ID NO: 12. In some embodiments, the nucleic acid encoding the dystrophin protein comprises the sequence of any one of SEQ ID NO: 2-3. In some embodiments, the nucleic acid encoding the dystrophin protein comprises the sequence of any one of SEQ ID NO: 6-7. In some embodiments, the cell is a myocyte. In some embodiments, the muscle cell comprises a skeletal muscle myocyte, a cardiomyocyte, or a smooth muscle cell. In some embodiments, the muscle cell is located within a muscle tissue of the subject, the method further comprising one or more of: applying heat to the muscle tissue, or increasing a blood flow rate in the muscle tissue. In some embodiments, heat is applied to the muscle tissue using a transcutaneous compress at a temperature of at least 37 C. In some embodiments, heat is applied to the muscle tissue using ultrasound energy. In some embodiments, a coding sequence of the nucleic acid encoding the full length dystrophin protein is contiguous. In some embodiments, the nucleic acid coding sequence of the functional dystrophin protein is contiguous. In some embodiments, at least 6, 7, 8, 9. 10, 11, 12, 13, 14, 15, 16, 17, 18, 19, 20, 21, 22, 23, 24, 25, 26, 27, 28, 29, 30, 35, 40, 45, 50, 55, 60, or 65% of muscle fibers in the muscle tissue of the subject contain the nucleic acid encoding the functional dystrophin protein. In some embodiments, at least 1% of muscle fibers in the muscle tissue of the subject express the dystrophin protein. In some embodiments, the cell or the myocyte is in a muscle tissue of the subject, wherein administering the sonoactive agent to the subject comprises distributing the sonoactive agent throughout the capillaries of the muscle tissue. In some embodiments, applying the ultrasound energy comprises applying an acoustic radiation force to the muscle tissue In some embodiments, applying the acoustic radiation force comprises displacing the muscle tissue In some embodiments, applying the acoustic radiation force comprises displacing a volume of blood in the capillaries of the muscle tissue In some embodiments, displacing the volume of blood in the capillaries of the muscle tissue transmits a pressure wave throughout a vascular system of the muscle tissue, thereby disrupting the sonoactive agent. In some embodiments, the pressure wave does and/or the ultrasound energy applying the acoustic radiation force does not induce vibrational cavitation of the sonoactive agent. In some embodiments, the muscle tissue is located in a heart or a diaphragm of the subject. In some embodiments, administering the ultrasound acoustic energy comprises applying a first ultrasound energy at a MI of up to 0.4, and a first second ultrasound energy at a MI of greater than 0.4 and up to 3.0. In some embodiments, administering the ultrasound acoustic energy comprises applying a first ultrasound energy at a MI of up to 0.4, and a first second ultrasound energy at a MI of 0.8-1.9. In some embodiments, administering the ultrasound acoustic energy comprises applying an acoustic radiation force to the subject. In some embodiments, the ultrasound energy is applied at an intensity (ISPTA) of at least 200 mW/cm². In some embodiments, the ultrasound energy displaces a muscle tissue of the subject by at least 0.1 mm. In some embodiments, the ultrasound energy is applied at a pulse length of at least 20 microseconds. In some embodiments, the sonoactive agent comprises a shell filled with a gas. In some embodiments, the shell comprises a protein stabilized shell or a lipid stabilized shell. In some embodiments, the gas comprises a perfluorinated case. In some embodiments, the sonoactive agent comprises microbubbles. In some embodiments, the sonoactive agent comprises nanobubbles. In some embodiments, the subject has a mutation in a gene encoding a dystrophin protein in a genome of the subject. In some embodiments, the subject has a dystrophinopathy disorder. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte. In some embodiments, administering the nucleic acid, administering the sonoactive agent, and applying the ultrasound energy induces expression of the dystrophin protein thereby treating the subject having the dystrophinopathy disorder. In some embodiments, the method reduces or ameliorates a symptom of the dystrophinopathy disorder in the subject. In some embodiments, the method further includes administering a second dose of the nucleic acid encoding the dystrophin protein, the sonoactive agent, and the ultrasound energy to the subject. In some embodiments, the subject is a mammalian subject. In some embodiments, the subject is a human subject. In some embodiments, the nucleic acid encoding the dystrophin protein is not comprised within a viral vector. In some embodiments, the subject is non-embryonic. In some embodiments, the subject weighs at least 5 kg

Aspects disclosed herein provide a nucleic acid comprising a nucleic acid sequence having at least 90% sequence identity to SEQ ID NO: 4. In some embodiments, the nucleic acid sequence has at least 91% sequence identity to SEQ ID NO: 4. In some embodiments, the nucleic acid sequence has at least 92% sequence identity to SEQ ID NO: 4. In some embodiments, the nucleic acid sequence has at least 93% sequence identity to SEQ ID NO: 4. In some embodiments, the nucleic acid sequence has at least 94% sequence identity to SEQ ID NO: 4. In some embodiments, the nucleic acid sequence has at least 95% sequence identity to SEQ ID NO: 4. In some embodiments, the nucleic acid sequence has at least 96% sequence identity to SEQ ID NO: 4. In some embodiments, the nucleic acid sequence has at least 97% sequence identity to SEQ ID NO: 4. In some embodiments, the nucleic acid sequence has at least 98% sequence identity to SEQ ID NO: 4. In some embodiments, the nucleic acid sequence has at least 99% sequence identity to SEQ ID NO: 4. In some embodiments, the nucleic acid sequence is a muscle tissue specific promoter sequence. In some embodiments, the nucleic acid sequence enhances expression of a therapeutic transgene in a muscle cell. In some embodiments, the muscle cell comprises a myocyte, a cardiomyocyte, or a smooth muscle cell. In some embodiments, the nucleic acid sequence facilitates expression of a transgene coupled to the promoter for at least 30, 60, 90, 120, 150, 180, 210, 240, 270, or 275 days. In some embodiments, the nucleic acid sequence is comprised within a nucleic acid delivery vector and coupled to a nucleic acid coding sequence encoding a therapeutic transgene, wherein the therapeutic transgene encodes a protein having at least 90% sequence identity to SEQ ID NO: 1. Aspects disclosed herein provide a nucleic acid delivery vector comprising the nucleic acid which is a muscle tissue specific promoter sequence having at least 90% sequence identity to SEQ ID NO: 4. Aspects disclosed herein provide a pharmaceutical composition comprising the nucleic acid which is a muscle tissue specific promoter sequence having at least 90% sequence identity to SEQ ID NO: 4. Aspects disclosed herein provide a cell comprising the nucleic acid which is a muscle tissue specific promoter sequence having at least 90% sequence identity to SEQ ID NO: 4. Aspects disclosed herein provide a kit comprising: 1) a sonoactive agent; and 2) means for expressing a therapeutic transgene in a subject. Aspects disclosed herein provide a kit comprising: 1) a sonoactive agent; and 2) the nucleic acid which is a muscle tissue specific promoter sequence having at least 90% sequence identity to SEQ ID NO: 4.

Aspects disclosed herein provide a nucleic acid comprising a nucleic acid sequence having at least 90% sequence identity to SEQ ID NO: 34. In some embodiments, the nucleic acid sequence has at least 91% sequence identity to SEQ ID NO: 34. In some embodiments, the nucleic acid sequence has at least 92% sequence identity to SEQ ID NO: 34. In some embodiments, the nucleic acid sequence has at least 93% sequence identity to SEQ ID NO: 34. In some embodiments, the nucleic acid sequence has at least 95% sequence identity to SEQ ID NO: 34. In some embodiments, the nucleic acid sequence has at least 97% sequence identity to SEQ ID NO: 34. In some embodiments, the nucleic acid sequence has at least 99% sequence identity to SEQ ID NO: 34. In some embodiments, the nucleic acid sequence is SEQ ID NO: 34. In some embodiments, the nucleic acid sequence encodes a dystrophin protein. In some embodiments, the nucleic acid sequence encodes a full length dystrophin protein. In some embodiments, the nucleic acid sequence encodes a dystrophin protein at least 3500 amino acids in length. In some embodiments, the nucleic acid sequence encodes a protein having at least 90% sequence identity to SEQ ID NO: 1. In some embodiments, the nucleic acid sequence comprises a muscle tissue specific promoter sequence. In some embodiments, the muscle tissue specific promoter sequence enhances expression of a dystrophin protein in a muscle cell. In some embodiments, the muscle cell comprises a myocyte, a cardiomyocyte, or a smooth muscle cell. In some embodiments, the nucleic acid sequence is an expression cassette for a dystrophin protein. In some embodiments, the nucleic acid sequence is an expression cassette for a dystrophin protein optimized for expression of the dystrophin protein in mammalian cells. In some embodiments, the nucleic acid sequence is an expression cassette for a dystrophin protein optimized for expression of the dystrophin protein in myocytes. In some embodiments, the nucleic acid sequence is comprised within a nucleic acid delivery vector having a sequence of SEQ ID NO: 17.

Aspects disclosed herein provide a nucleic acid sequence comprising a sequence having at least 80% sequence identity to any one of SEQ ID NO: 2-3. In some embodiments, the nucleic acid sequence has at least 81, 82, 83, 84, 85, 86, 87, 88, 89, 90, 91, 92, 93, 94, 95, 96, 97, 98, or 99% sequence identity to any one of SEQ ID NO: 2-3. In some embodiments, the nucleic acid sequence is any one of SEQ ID NO: 2-3. In some embodiments, the nucleic acid sequence encodes a dystrophin protein. In some embodiments, the nucleic acid sequence encodes a full length dystrophin protein. In some embodiments, the nucleic acid sequence encodes a dystrophin protein at least 3500 amino acids in length. Aspects disclosed herein provide a nucleic acid delivery vector comprising the nucleic acid of embodiments disclosed herein. Aspects disclosed herein provide a use of the nucleic acids of any one of embodiments disclosed herein in a method of treating a subject having a dystrophinopathy disorder requiring a dystrophin gene therapy or a dystrophin protein replacement therapy. Aspects disclosed herein provide a pharmaceutical composition comprising the nucleic acid of embodiments disclosed herein. Aspects disclosed herein provide a use of the pharmaceutical composition of embodiments disclosed herein in a method of treating a subject having a dystrophinopathy disorder requiring a dystrophin gene therapy or a dystrophin protein replacement therapy. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte. Aspects disclosed herein provide a cell comprising the nucleic acid of embodiments disclosed herein.

Aspects disclosed herein provide a nucleic acid comprising a nucleic acid sequence having at least 80% sequence identity to any one of SEQ ID NO: 4-5. In some embodiments, the nucleic acid has at least 81, 82, 83, 84, 85, 86, 87, 88, 89, 90, 91, 92, 93, 94, 95, 96, 97, 98, or 99% sequence identity to any one of SEQ ID NO: 4-5. In some embodiments, the nucleic acid is a muscle tissue specific promoter sequence. In some embodiments, the nucleic acid which comprises the muscle specific promoter sequence facilitates expression of a transgene coupled to the promoter for at least 30, 60, 90, 120, 150, 180, 210, 240, 270, or 275 days. In some embodiments, a nucleic acid delivery vector comprises the muscle specific promoter sequence facilitates expression of a transgene coupled to the promoter for at least 30, 60, 90, 120, 150, 180, 210, 240, 270, or 275 days. In some embodiments, the nucleic acid enhances expression of a therapeutic transgene in a muscle cell. In some embodiments, the muscle cell comprises a myocyte, a cardiomyocyte, or a smooth muscle cell. Aspects disclosed herein provide a nucleic acid delivery vector comprising the nucleic acid of any one of embodiments disclosed. Aspects disclosed herein provide a use of the nucleic acid of any one of embodiments disclosed in a method of treating a subject having a dystrophinopathy disorder requiring a dystrophin gene therapy or a dystrophin protein replacement therapy. Aspects disclosed herein provide a pharmaceutical composition comprising the nucleic acid of any one of embodiments disclosed. Aspects disclosed herein provide a use of the pharmaceutical composition of embodiments disclosed in a method of treating a subject having a dystrophinopathy disorder requiring a dystrophin gene therapy or a dystrophin protein replacement therapy. Aspects disclosed herein provide a cell comprising the nucleic acid of any one of embodiments disclosed. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte.

Aspects disclosed herein provide a nucleic acid comprising a promoter sequence having at least 90% sequence identity to SEQ ID NO: 18. In some embodiments, the promoter sequence has at least 91, 92, 93, 94, 95, 96, 97, 98, or 99% sequence identity to SEQ ID NO: 18. In some embodiments, the promoter sequence is SEQ ID NO: 18.

Aspects disclosed herein provide a nucleic acid comprising a sequence which has at least 80% sequence identity to any one of SEQ ID NO: 16-17. Aspects disclosed herein provide a nucleic acid comprising a sequence of SEQ ID NO: 16-17.

In some embodiments, a coding sequence of the nucleic acid encoding the full length dystrophin protein is contiguous. In some embodiments, the nucleic acid coding sequence of the functional dystrophin protein is contiguous. In some embodiments, at least 6, 7, 8, 9. 10, 11, 12, 13, 14, 15, 16, 17, 18, 19, 20, 21, 22, 23, 24, 25, 26, 27, 28, 29, 30, 35, 40, 45, 50, 55, 60, or 65% of muscle fibers in the muscle tissue of the subject contain the nucleic acid encoding the functional dystrophin protein. In some embodiments, at least 1% of muscle fibers in the muscle tissue of the subject express the dystrophin protein. In some embodiments, the cell or the myocyte is in a muscle tissue of the subject, wherein administering the sonoactive agent to the subject comprises distributing the sonoactive agent throughout the capillaries of the muscle tissue. In some embodiments, applying the ultrasound energy comprises applying an acoustic radiation force to the muscle tissue In some embodiments, applying the acoustic radiation force comprises displacing the muscle tissue In some embodiments, applying the acoustic radiation force comprises displacing a volume of blood in the capillaries of the muscle tissue In some embodiments, displacing the volume of blood in the capillaries of the muscle tissue transmits a pressure wave throughout a vascular system of the muscle tissue, thereby disrupting the sonoactive agent. In some embodiments, the pressure wave does and/or the ultrasound energy applying the acoustic radiation force does not induce vibrational cavitation of the sonoactive agent.

Aspects disclosed herein provide a kit comprising: 1) a sonoactive agent; and 2) means for expressing a functional dystrophin protein in a subject. Aspects disclosed herein provide a kit comprising: 1) a sonoactive agent; and 2) a nucleic acid of any one of SEQ ID NO: 1-50. Aspects disclosed herein provide a kit comprising: 1) a sonoactive agent; and 2) the nucleic acid of SEQ ID NOS: 2-7, 34-36, or 37-40. In some embodiments, the kit further comprises instructions for administering ultrasound acoustic energy to a subject to facilitate expression of the functional dystrophin protein in a subject. In some embodiments, the kit further comprises instructions for administering ultrasound acoustic energy to a subject to facilitate expression of the nucleic acid in a subject. In some embodiments, the sonoactive agent comprises lipid-stabilized microstructures. In some embodiments, wherein the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble. In some embodiments, the sonoactive agent comprises protein stabilized microstructures. In some embodiments, the protein stabilized microstructures comprise an albumin stabilized shell surrounding a perfluorinated gas core. In some embodiments, the protein stabilized microstructures are Optison microbubbles.

Aspects disclosed herein provide a nucleic acid comprising a sequence which has at least 80% sequence identity to any one of SEQ ID NO: 1-50. Aspects disclosed herein provide a nucleic acid comprising a sequence of SEQ ID NO: 1-50.

Aspects disclosed herein provide a nucleic acid comprising a promoter sequence having at least 90% sequence identity to any one of SEQ ID NOS: 37-40. In some embodiments, the promoter sequence comprises at least 91, 92, 93, 94, 95, 96, 97, 98, or 99% sequence identity to any one of SEQ ID NOS: 37-40. In some embodiments, the promoter sequence comprises any one of SEQ ID NOS: 37-40. In some embodiments, the promoter sequence is configured to increase expression of a nucleic acid encoding a therapeutic protein operably coupled to the promoter sequence. In some embodiments, the promoter sequence is configured to increase expression of a nucleic acid encoding a therapeutic protein operably coupled to the promoter sequence in multiple cell types. In some embodiments, the promoter sequence is configured to increase expression of the nucleic acid encoding the therapeutic protein in a muscle cell. In some embodiments, the promoter sequence is configured to increase expression of the nucleic acid encoding the therapeutic protein in a kidney cell. In some embodiments, the promoter sequence is configured to increase expression of the nucleic acid encoding the therapeutic protein in a liver cell. In some embodiments, the liver cell is a hepatocyte, or a LSEC. In some embodiments, the promoter sequence is configured to increase expression of the nucleic acid encoding the therapeutic protein in a myocyte. Aspects disclosed herein provide nucleic acid delivery vector comprising the nucleic acid of any one of the present embodiments disclosed herein. Aspects disclosed herein provide pharmaceutical composition comprising the nucleic acid of any one of the present embodiments disclosed herein Aspects disclosed herein provide cell comprising the nucleic acid of any one of the present embodiments disclosed herein. Aspects disclosed herein provide method of treating a subject comprising administering the nucleic acid of any one of the present embodiments disclosed herein. In some embodiments, the method further includes administering ultrasound to the subject. In some embodiments, the method further includes administering a sonoactive agent to the subject. In some embodiments, the method further includes administering a lipid nanoparticle or a polymer nanoparticle to the subject. In some embodiments, the nucleic acid is contained within a viral vector.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary ultrasound transducer system having computer processors with a computer readable medium storing instructions for implementing the methods of the present disclosure;

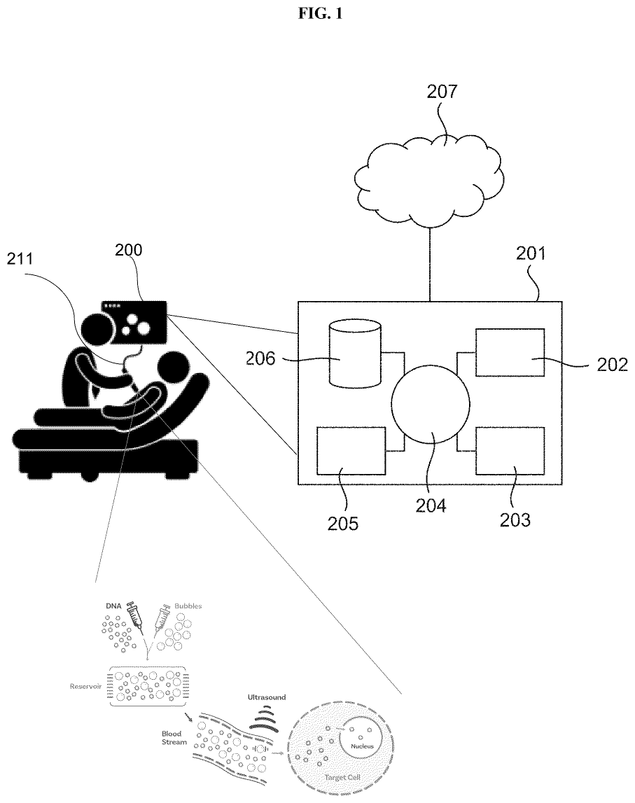

FIG. 2 shows results of a western blot assay illustrating an antibody highly specific to human dystrophin with no cross reactivity to murine dystrophin;

FIG. 3 shows results of a western blot assay illustrating in-vitro expression data of expression of various codon optimized human dystrophin sequences;

FIG. 4 shows results of a chemiluminescence immunoassay illustrating in-vitro expression data of expression of various codon optimized human dystrophin sequences;

FIG. 5 shows in-vivo data fluorescence data illustrating expression of a gene encoding a reporter protein downstream of a promoter sequence of the present disclosure;

FIG. 6 shows results of a western blot assay illustrating expression of full length human dystrophin protein in-vivo;

FIG. 7 illustrates a human dystrophin protein and the epitopes targeted by various antibodies used to detect human dystrophin;

FIG. 8 shows results of a western blot assay illustrating expression of full length human dystrophin protein in-vivo;

FIG. 9 shows in-vivo fluorescence data illustrating expression of a gene encoding a reporter protein downstream of a promoter sequence of the present disclosure;

FIG. 10 shows results of a western blot assay illustrating expression of full length human dystrophin protein in-vivo;

FIG. 11 shows results of a western blot assay illustrating expression of full length human dystrophin protein in-vivo;

FIG. 12 shows immunohistochemistry stain images illustrating expression of full length human dystrophin protein in-vivo in transfected cells, arrows indicate cells expressing human dystrophin protein;

FIG. 13 shows immunohistochemistry stain images illustrating expression of full length human dystrophin protein in-vivo in transfected cells, arrows indicate cells expressing human dystrophin protein;

FIG. 14 shows immunohistochemistry stain images illustrating expression of full length human dystrophin protein in-vivo in transfected cells, arrows indicate cells expressing human dystrophin protein;

FIG. 15 shows immunohistochemistry stain images illustrating delivery of nucleic acids encoding length human dystrophin protein in-vivo in transfected tissue;

FIG. 16 shows results of a digital pathology analysis quantifying delivery of a nucleic acid of the present disclosure in a population of muscle fibers from treated muscle tissue;

FIG. 17 shows in-vivo data fluorescence data illustrating expression of a gene encoding a reporter protein downstream of a promoter sequence of the present disclosure;

FIG. 18 shows immunohistochemistry stain images illustrating delivery of nucleic acids encoding length human dystrophin protein in-vivo throughout transfected tissue;

FIG. 19 shows results of a digital pathology analysis quantifying delivery of a nucleic acid of the present disclosure in a population of muscle fibers from treated muscle tissue;

FIG. 20 shows results of a copy number analysis quantifying delivery of a nucleic acid of the present disclosure in a population of muscle cells from treated muscle tissue;

FIG. 21 shows results of a western blot assay assessing expression of full length dystrophin and normalized for total protein expression compared to an untreated control;

FIG. 22 shows results of a western blot assay illustrating expression of full length dystrophin protein in-vivo;

FIG. 23 results of a copy number analysis quantifying delivery of a nucleic acid of the present disclosure in a population of muscle cells from treated muscle tissue;

FIG. 24 shows immunohistochemistry stain images illustrating delivery of nucleic acids encoding length human dystrophin protein in-vivo throughout transfected tissue;

FIG. 25 shows results of a digital pathology analysis quantifying delivery of a nucleic acid of the present disclosure in a population of muscle fibers from treated muscle tissue;

FIG. 26 shows in-vivo fluorescence data illustrating expression of a gene encoding a reporter protein downstream of a promoter sequence of the present disclosure;

FIG. 27 shows in-vivo data fluorescence data illustrating expression of a gene encoding a reporter protein downstream of a promoter sequence of the present disclosure;

FIG. 28 shows results of a western blot assay illustrating expression of full length human dystrophin protein in-vivo with a promoter sequence of the present disclosure; and

FIG. 29 shows in-vivo data fluorescence data illustrating expression of a gene encoding a reporter protein downstream of a promoter sequence of the present disclosure.

### DETAILED DESCRIPTION

Dystrophinopathy disorders such as Duchenne muscular dystrophy (DMD), Becker muscular dystrophy (BMD), and DMD-associated dilated cardiomyopathy (DCM) are genetic disorders associated with disfunction of the dystrophin gene in mammalian subjects, and are among the most devastating genetic disorders of our time. Lacking a safe and effective treatment, affected subjects generally begin to experience symptoms of progressive muscle fiber degeneration and weakness in mid-childhood to teenage years, with most DMD patients having a life expectancy of only 18 to 25 years. While various gene therapy approaches attempt to a treat dystrophinopathy disorders within the limitations of current technology, such as with delivery of dystrophin truncates or dystrophin fusion proteins, there are no therapies which improve the functional outcome of affected patients by delivering a gene therapy which results in the production of a functional copy of the dystrophin protein.

Dystrophinopathy disorders are exceptionally difficult genetic disorders to treat due to the many challenges associated with attempting to deliver and express a functional copy of the dystrophin protein in affected subjects. Extensive muscle mass in skeletal muscles and heart are difficult to target with viral vectors, with most viral vectors poorly penetrating through vascular tissue to reach myocytes, and significant immune response to viral vectors in the skeletal muscles and heart further reducing their ability target these tissues. In addition, dystrophin is among the largest genes in the human genome at over 3500 amino acids, and far exceeds the carrying capacity of viral vectors for gene replacement therapies. Other gene therapy approaches such as gene editing with CRISPR or exon skipping are disfavored modalities for correction of the dystrophin gene because they would need to be tailored for specific mutations in view of the large length of the dystrophin gene, limiting the broad applicability of any such therapy. Further, the early on-set of most dystrophinopathy disorders also requires effective treatments to start in childhood, which significantly limits the options for readministering the gene therapy, and complicating development efforts. Proposed herein are methods of expressing a functional dystrophin protein in a cell of a subject using an ultrasound based delivery modality which is highly effective at inducing expression of the dystrophin protein, while also avoiding many of the problems associated with prior approaches such as the lack of general applicability, the inability to transfect a large or full length dystrophin protein, inability to repeatedly dose subjects, and safety/toxicity concerns.

Aspects disclosed herein provide a method of expressing a nucleic acid encoding a dystrophin protein in a cell of a subject, comprising: administering the nucleic acid encoding the dystrophin protein to the subject, administering a sonoactive agent to the subject, and applying ultrasound energy the cell. Aspects disclosed herein provide a method of treating a dystrophinopathy disorder in a subject in need thereof comprising administering to the subject a nucleic acid encoding a functional dystrophin protein, delivering the nucleic acid to a myocyte in a muscle tissue of the subject along a length of a muscle fiber in the muscle tissue and/or about the sarcolemma of a muscle fiber in the muscle tissue, thereby treating the dystrophinopathy disorder. Aspects disclosed herein provide a method of expressing dystrophin in a subject comprising: inducing uptake by a cell in a population of cells of an expression vector encoding a functional dystrophin protein or a functional fragment thereof, in which at least 0.25% of cells in the population of cells uptake the expression vector. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject in an amount exceeding 0.5% of a dystrophin level in a wild type subject not having the dystrophinopathy disorder, thereby treating the dystrophinopathy disorder. Aspects disclosed herein provide a use of a nucleic acid encoding a functional dystrophin protein and a sonoactive agent in a method of treating a subject having a dystrophinopathy disorder requiring a dystrophin gene therapy or a dystrophin protein replacement therapy. Aspects disclosed herein provide a method of delivering a nucleic acid encoding a functional dystrophin protein in a muscle cell in a muscle tissue of a subject, comprising: administering the nucleic acid encoding the dystrophin protein to the subject wherein a coding sequence of the nucleic acid encoding the dystrophin protein is at least 10000 nucleotides; administering a sonoactive agent to the subject; and applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue thereby disrupting the sonoactive agent and delivering the nucleic acid encoding the dystrophin protein to at least 5% of muscle fibers in the muscle tissue.

In some embodiments, the cells comprise a myocyte, and wherein the functional dystrophin protein or a functional fragment thereof is expressed in the myocyte in an amount exceeding 0.5% of a dystrophin level in a wild type subject. In some embodiments, the cells comprise a myocyte, and wherein the functional dystrophin protein or a functional fragment thereof is expressed (i) about the cellular membrane of the myocyte, (ii) throughout the cytoplasm of the myocyte, or (iii) both about the cellular membrane and throughout the cytoplasm of the myocyte. In some embodiments, the functional dystrophin protein positioned about the sarcolemma is located along a surface of the sarcolemma along a length of the sarcolemma in a direction parallel to the muscle fiber encapsulated within the sarcolemma. In some embodiments, the cell is a myocyte, wherein the dystrophin protein or a functional fragment thereof is expressed in the myocyte in an amount exceeding 0.5% of a dystrophin level in a wild type subject. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject about the cellular membrane of the myocyte, throughout the cytoplasm of the myocyte, or both about the about the cellular membrane and throughout the cytoplasm of the myocyte, thereby treating the dystrophinopathy disorder. Aspects disclosed herein provide a method of expressing dystrophin in a subject comprising: inducing uptake by a cell in a diaphragm of a subject of an expression vector encoding a functional dystrophin protein or a functional fragment thereof. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not conjugated to an antibody. Aspects disclosed herein provide a method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not associated with cationic peptide moieties. Aspects disclosed herein provide a method of treating a dystrophinopathy disorder in a subject in need thereof comprising administering to the subject a nucleic acid encoding a functional dystrophin protein, transfecting the nucleic acid to a muscle tissue of the subject, wherein at least 5% of muscle fibers in the muscle tissue of the subject contain the nucleic acid encoding the functional dystrophin protein.

Dystrophin is a critical structural protein essential for maintaining the integrity and functionality of muscle cells. Encoded by a gene located on the X chromosome, dystrophin's absence or dysfunction leads to severe muscle disorders like Duchenne muscular dystrophy (DMD) and Becker muscular dystrophy (BMD). Structurally, dystrophin is a large rod-shaped protein with a molecular weight of approximately 427 kDa. It consists of several domains: the N-terminal actin-binding domain (ABD), which anchors dystrophin to the intracellular actin cytoskeleton and provides mechanical support; the central rod domain, made up of 24 spectrin-like repeats and hinge regions, giving the protein its flexibility and elasticity; the cysteine-rich domain, which connects dystrophin to β-dystroglycan in the sarcolemma via the dystroglycan complex; and the C-terminal domain, which interacts with syntrophins and dystrobrevin to form and stabilize the dystrophin-associated protein complex (DAPC). Functionally, dystrophin serves as a mechanical stabilizer and a signaling hub within muscle cells. It links the internal actin cytoskeleton to the extracellular matrix (ECM) via the DAPC, distributing mechanical forces generated during muscle contraction and protecting the sarcolemma from damage. This connection maintains sarcolemma integrity, preventing ruptures and excessive permeability. Additionally, dystrophin facilitates signal transduction by interacting with various proteins in the DAPC, modulating cellular pathways critical for muscle repair, regeneration, and stress response. The protein also contributes to calcium homeostasis by stabilizing membrane channels that control calcium influx, thereby protecting muscle cells from calcium-mediated damage. Furthermore, dystrophin helps mitigate fibrosis and inflammation, which are consequences of sarcolemma instability in its absence.

It is proposed that one possible reason for the failure of prior gene therapies to produce a functional copy of a dystrophin protein is due to the inability to deliver a full length dystrophin protein, or a fragment thereof which approaches a full length dystrophin protein. Most AAV vectors seeking to delivery dystrophin truncates only result in expression of dystrophin truncates of less than 1500 amino acids, far fewer than the 3500 amino acids of the native protein. Similarly, other AAV and viral vectors seeking to delivery dystrophin fragments for formation of a fusion protein also fail to form functional dystrophin protein in the subject. As is disclosed herein, the methods disclosed herein provide a beneficial technical effect of allowing for the delivery of gene therapies encoding large and, in some cases, full length dystrophin protein. In some cases, the expression of such large and/or full length dystrophin proteins in subjects can provide beneficial technical effect of treating or ameliorating a symptom of a dystrophinopathy disorders where prior gene therapies have failed to do so.

In some embodiments, the method induces uptake of the nucleic acid into at least 0.25% of a population of cells comprising the cell. In some embodiments, the population of cells is in a cardiac tissue or a heart of the subject, a skeletal muscle tissue or a skeletal muscle of the subject, or a diaphragm tissue or a diaphragm of the subject. In some embodiments, the cell is a multinucleated cell, a myocyte or a muscle cell. In sone embodiments, the functional dystrophin protein or a functional fragment thereof is expressed about the cellular membrane of the myocyte, throughout the cytoplasm of the myocyte, or both about the about the cellular membrane and throughout the cytoplasm of the myocyte. In some embodiments, the myocyte or the muscle cell comprises a skeletal muscle myocyte, a cardiomyocyte, or a smooth muscle cell. In some embodiments, the myocyte or the muscle cell is located in a cardiac tissue or a heart of the subject. In some embodiments, dystrophin is expressed in the cardiac tissue or the heart in an amount of at least 0.3, 0.5, 0.75, 1, 2, 3, 4, 5, 6, 7, 8, 9, or 10% of a dystrophin level in human heart whole tissue lysate as measured by western blot assay. In some embodiments, the myocyte or the muscle cell is located in a skeletal muscle tissue or a skeletal muscle of the subject. In some embodiments, the dystrophin is expressed in the muscle tissue or the skeletal muscle in an amount of at least 0.3, 0.5, 0.75, 1, 2, 3, 4, 5% of a dystrophin level in human skeletal muscle whole tissue lysate when measured by western blot assay. In some embodiments, the myocyte or the muscle cell is located in a diaphragm tissue or a diaphragm of the subject. In some embodiments, the dystrophin is expressed in the diaphragm tissue or the diaphragm in an amount of at least 0.3, 0.5, 0.75, 1, 2, 3, 4, 5, 6, 7, 8, 9, or 10% of a dystrophin level in human diaphragm whole tissue lysate when measured by western blot assay. In some embodiments, the dystrophin is expressed in the cardiac tissue, the muscle tissue, or the diaphragm tissue, throughout a treated organ comprising the cardiac tissue, the muscle tissue, or the diaphragm tissue. In some embodiments, the dystrophin is expressed at detectable levels throughout the treated organ in two or more samples taken from opposite ends of the treated organ. In some embodiments, the nucleic acid or the expression vector is an unencapsulated nucleic acid. In some embodiments, a coding sequence of the nucleic acid encoding the full length dystrophin protein is contiguous. In some embodiments, the nucleic acid coding sequence of the functional dystrophin protein is contiguous. In some embodiments, at least 6, 7, 8, 9. 10, 11, 12, 13, 14, 15, 16, 17, 18, 19, 20, 21, 22, 23, 24, 25, 26, 27, 28, 29, 30, 35, 40, 45, 50, 55, 60, or 65% of muscle fibers in the muscle tissue of the subject contain the nucleic acid encoding the functional dystrophin protein. In some embodiments, at least 1% of muscle fibers in the muscle tissue of the subject express the dystrophin protein.

Aspects disclosed herein provide a method of delivering a nucleic acid encoding a functional dystrophin protein in a muscle cell in a muscle tissue of a subject, comprising: administering the nucleic acid encoding the dystrophin protein to the subject wherein a coding sequence of the nucleic acid encoding the dystrophin protein is at least 10000 nucleotides; administering a sonoactive agent to the subject; and applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue thereby disrupting the sonoactive agent and delivering the nucleic acid encoding the dystrophin protein to at least 5% of muscle fibers in the muscle tissue. In some embodiments, the method includes applying heat to the muscle tissue and increasing a perfusion of the nucleic acid and the sonoactive agent in the muscle tissue before administering the ultrasound. In some cases, the methods of administration and the ultrasound mediated delivery described herein which includes perfusing the muscle tissue with the sonoactive agent and the nucleic acid and then applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue provides a beneficial technical effect in disrupting the sonoactive agent throughout the muscle tissue and delivering the nucleic acid encoding the dystrophin protein to at least 5% of muscle fibers in the muscle tissue. In some cases, the method further provides a technical benefit of properly localizing the functional dystrophin protein positioned about the sarcolemma along a surface of the sarcolemma along a length of the sarcolemma in a direction parallel to the muscle fiber encapsulated within the sarcolemma.

In some embodiments, a coding sequence of the functional dystrophin protein is at least 5000, 6000, 7000, 8000, 9000, 10000, or 11000 nucleotides. In some embodiments, the method further includes administering a s agent to the subject, and applying ultrasound energy a cell of the subject. In some embodiments, a length the functional dystrophin protein is at least 1750, 2000, 2250, 2500, 2750, 3000, or 3250 amino acids. In some embodiments, a coding sequence of the functional dystrophin protein is at least 5000 nucleotides. In some embodiments, the nucleic acid encoding the functional dystrophin protein and the sonoactive agent are configured to induce expression of the functional dystrophin protein in a cell of the subject upon administration of ultrasound energy to the cell. In some embodiments, the nucleic acid encodes a full length dystrophin protein. In some embodiments, a coding sequence of the nucleic acid encoding the full length dystrophin protein is at least 5000, 6000, 7000, 8000, 9000, 10000, or 11000 nucleotides. In some embodiments, a length the functional dystrophin protein is at least 1750, 2000, 2250, 2500, 2750, 3000, or 3250 amino acids. In some embodiments, the myocyte is a skeletal muscle myocyte and wherein dystrophin is expressed in the myocyte at a level of at least 1, 2, 3, 4, 5, 10, 20, 30, 40, 50, 60, 70, 80, 90, 100, 110, or 120% of a normal dystrophin level in a subject not having a dystrophinopathy disorder. A normal dystrophin level in skeletal muscle tissue generally refers to a dystrophin level in human skeletal muscle whole tissue lysate. In some embodiments, a coding sequence of the dystrophin protein in the nucleic acid encoding the dystrophin protein is at least 6000, 7000, 8000, 9000, 10000, or 11000 nucleotides. In some embodiments, the coding sequence of the dystrophin protein encodes a functional dystrophin protein. In some embodiments, the dystrophin protein is at least 1750, 2000, 2250, 2500, 2750, 3000, or 3250 amino acids in length. In some embodiments, the coding sequence of the functional dystrophin protein encodes a full length dystrophin protein. In some embodiments, the functional dystrophin protein is at least 1750, 2000, 2250, 2500, 2750, 3000, or 3250 amino acids in length. In some embodiments, the nucleic acid encoding the dystrophin protein is not comprised within a viral vector. In some embodiments, a coding sequence of the nucleic acid encoding the full length dystrophin protein is contiguous. In some embodiments, the nucleic acid coding sequence of the functional dystrophin protein is contiguous. In some embodiments, at least 6, 7, 8, 9. 10, 11, 12, 13, 14, 15, 16, 17, 18, 19, 20, 21, 22, 23, 24, 25, 26, 27, 28, 29, 30, 35, 40, 45, 50, 55, 60, or 65% of muscle fibers in the muscle tissue of the subject contain the nucleic acid encoding the functional dystrophin protein. In some embodiments, at least 1% of muscle fibers in the muscle tissue of the subject express the dystrophin protein. In some embodiments, a functional dystrophin protein is a dystrophin protein which is effective at reducing or ameliorating a symptom of the dystrophinopathy disorder in a subject.

It is further proposed that an additional reason for the failure of prior gene therapies to produce a functional copy of a dystrophin protein is due to the inability to correct disease phenotypes associated with dystrophinopathy disorder is the failure of micro-dystrophin transgene products to properly be delivered or localize within treated myocytes in a manner which fulfills the mechanical function of an endogenously produced dystrophin protein in healthy subjects, for example, due to failure to localize to the inner (cytoplasmic) surface of the sarcolemma and link the internal actin cytoskeleton to the extracellular matrix (ECM) and distributing mechanical forces generated during muscle contraction. Aspects disclosed herein provide a method of treating a dystrophinopathy disorder in a subject in need thereof comprising administering to the subject a nucleic acid encoding a functional dystrophin protein, delivering the nucleic acid to a myocyte in a muscle tissue of the subject along a length of a muscle fiber in the muscle tissue and/or about the sarcolemma of a muscle fiber in the muscle tissue, thereby treating the dystrophinopathy disorder. In some embodiments, the functional dystrophin protein is expressed in the myocyte in the muscle tissue. In some embodiments, the functional dystrophin protein is expressed along the length of the muscle fiber in the muscle tissue. In some embodiments, the functional dystrophin protein is about the sarcolemma of the muscle fiber in the muscle tissue. In some embodiments, the functional dystrophin protein transfers tension along the length of the muscle fiber or improves a maximum tension loading capacity of the muscle tissue. In some embodiments, at least 1, 3, 5, 10, 15, 20, 25, or 28% of the muscle fibers in the treated muscle tissue are contain the nucleic acid encoding the functional dystrophin protein. In some cases, the methods of ultrasound mediated delivery described herein provide a beneficial technical effect in delivering the nucleic acid to a myocyte in a muscle tissue of the subject along a length of a muscle fiber in the muscle tissue and/or about the sarcolemma of a muscle fiber in the muscle tissue. In some embodiments, along the length of a muscle fiber refers to positioning along the longitudinal (axial) of the muscle fiber sarcolemma, running parallel to the axial direction of the muscle fibers. In some cases, the methods of ultrasound mediated delivery described herein provide a beneficial technical effect in expressing the functional dystrophin protein in a myocyte in a muscle tissue of the subject along a length of a muscle fiber in the muscle tissue and/or about the sarcolemma of a muscle fiber in the muscle tissue.

Disclosed herein are optimized vectors for promoting expression of a dystrophin in a myocyte of a subject. In some embodiments, the nucleic acid encoding the dystrophin protein further comprises a promoter sequence. In some cases, the promoter sequence provides a beneficial technical effect of enhancing expression of the dystrophin in a myocyte of a subject. In some cases, the promoter sequence provides a beneficial technical effect of reducing off-target expression of the dystrophin in cells other than myocytes in the subject. In some embodiments, the promoter sequence comprises a sequence of any one of SEQ ID NO: 4-5, or 18.

In some embodiments, the nucleic acid delivery vectors disclosed herein comprise a Woodchuck Hepatitis Virus Posttranscriptional Regulatory Element 3 (WPRE3). In some cases, the WPRE3 element improves ribosomal recruitment to a transcribed mRNA, enhances translation initiation of a transcribed mRNA, and promotes the expression of the therapeutic nucleic acid sequence. In some cases, the WPRE3 element contains sequences which interacts with cellular translation initiation factors, such as eIF4E (eukaryotic translation initiation factor 4E) and eIF4G (eukaryotic translation initiation factor 4G) and enhances the assembly of the translation initiation complex, facilitating the recruitment of ribosomes to the mRNA. In some cases, the WPRE3 element promotes efficient scanning of ribosomes along the mRNA, increasing initiation of translation at the correct site, and promoting the expression of the therapeutic nucleic acid sequence in the cell. In some cases, the translated WPRE3 element contains sequences that are recognized by ribosomal RNA or elements that stabilize the interaction between ribosomal subunits and the mRNA. In some cases, the translated WPRE3 element can reduce secondary mRNA structures such as hairpins and stem-loops, which can hinder ribosomal recruitment and reduce the efficiency of translation. In some cases, the translated WPRE3 element interacts with ribosomal proteins, promoting their binding to the mRNA, and increasing the stability of association between ribosomes and the mRNA. In some cases, the nucleic acid delivery vectors of the present disclosure may comprise one or more genetic regulatory elements including promoters, enhancers, polyadenylation signals, terminator sequences, transcriptional regulatory elements, nuclear targeting sequences, and viral sequences. In some cases, the nucleic acid delivery vectors or the present disclosure may comprise a promoter sequence operatively linked to a therapeutic nucleic acid sequence. In some cases, the nucleic acid delivery vectors or the present disclosure may comprise a polyadenylation sequence operatively linked to a therapeutic nucleic acid sequence. In some cases, the nucleic acid delivery vectors of the present disclosure may comprise an intron sequence operatively linked to a therapeutic nucleic acid sequence. In some cases, the nucleic acid delivery vectors of the present disclosure may comprise a nuclear targeting sequence positioned downstream of the therapeutic nucleic acid sequence. In some cases, the nucleic acid delivery vectors of the present disclosure may comprise a nuclear targeting sequence positioned downstream of the transgene and other regulatory elements, for example, a post transcriptional regulatory element, a polyadenylation signal, and/or other regulatory element. In some cases, the nucleic acid delivery vectors may comprise promoter sequences positioned upstream of the therapeutic nucleic acid sequence. In some embodiments, the nucleic acid delivery vectors may comprise an intron sequence positioned in between a promoter sequence and a therapeutic nucleic acid sequence. In some cases, the nucleic acid delivery vectors may comprise a post transcriptional regulatory element positioned downstream of a therapeutic nucleic acid sequence. In some cases, the nucleic acid delivery vectors may comprise polyadenylation sequence positioned downstream of a therapeutic nucleic acid sequence. In some cases, the nucleic acid delivery vectors may comprise a nuclear targeting sequence positioned downstream of a therapeutic nucleic acid sequence. In some cases, the nucleic acid delivery vectors comprise a WPRE3 element positioned downstream of the transgene. In some cases, the nucleic acid delivery vector comprises a bGH-poly A element position downstream of the WPRE3 element. In some cases, the WPRE3 element is positioned in between the therapeutic nucleic acid sequence and the bGH-poly A element. In some cases, the WPRE3 element is operably linked to the WPRE3 element and the bGH-poly A element. In some cases, the nuclear targeting sequence is positioned downstream of the bGH-polyA element. In some cases, the nuclear targeting sequence is operatively linked to the bGH-poly A element. In some cases, the nucleic acid delivery of vectors of the present disclosure may comprise a post transcriptional regulatory element operatively linked to the therapeutic nucleic acid sequence. In some cases, the nucleic acid delivery vectors of the present disclosure may comprise a nuclear targeting sequence operatively linked to a therapeutic nucleic acid sequence. In some cases, the nucleic acid delivery vectors of the present disclosure may comprise one or more viral sequences operatively linked to a therapeutic nucleic acid sequence. In some embodiments, the nucleic acid encoding the dystrophin protein further comprises a post transcriptional regulatory element. In some embodiments, the post transcriptional regulatory element comprises the sequence of any one of SEQ ID NO: 8-10. In some embodiments, the post transcriptional regulatory element and the nuclear targeting sequence comprise a sequence of SEQ ID NO: 12. Aspects disclosed herein provide a nucleic acid delivery vector comprising: a therapeutic nucleic acid sequence encoding a peptide having an amino acid sequence of SEQ ID NO:1; a first regulatory element comprising a sequence of SEQ ID NO: 51 positioned upstream of the therapeutic nucleic acid sequence; and a second regulatory element comprising a sequence of SEQ ID NO: 8 positioned downstream of the therapeutic nucleic acid sequence. In some embodiments, the second regulatory element comprises a sequence of SEQ ID NO: 8 upstream of SEQ ID NO: 9. In some embodiments, the nucleic acid delivery vector further comprises a nuclear targeting sequence having a sequence of SEQ ID NO: 11. In some embodiments, the nuclear targeting sequence is positioned downstream of the second regulatory element.

In some embodiments, the nucleic acid encoding the dystrophin protein further comprises a nuclear targeting sequence. The delivery vectors disclosed herein may comprise DNA nuclear targeting sequences (DTS) that enhance delivery of the nucleic acid composition and/or the therapeutic nucleic acid sequence to a cell. In some cases, the DTS sequences may comprise recognition sequences for endogenous DNA-binding proteins which lead to increased transfection efficiency of non-viral gene delivery by virtue of enhanced nuclear import of the nucleic acid composition, which can lead to enhanced transgene expression. In some cases, the DTS sequences are recognized by nuclear transport proteins, such as importins, bind the DTS sequences and form an import complex which shields the delivery vector from cytoplasmic nucleases during movement toward the nuclear envelope, and facilitate translocation of the delivery vector through the nuclear pore complex. In some cases, the enhanced nuclear import of the nucleic acid composition avoids degradation of the nucleic acid composition in the cytoplasm, brings nucleic acid composition closer in proximity to the transcriptional machinery, thereby promoting efficient transcription of the delivered transgene. In some cases, the DTS sequence minimizes the loss of transgene during cell division, and increases the likelihood of stable inheritance of the transgene by one or more daughter cells. Various DNA nuclear targeting sequences can be operably linked with nucleic acid sequences comprising the transgene of interest in the delivery vectors disclosed herein. In some embodiments, the nuclear targeting sequence comprise a sequence of SEQ ID NO: 11. In some embodiments, the nucleic acid encoding the dystrophin protein further comprises a post transcriptional regulatory element and a nuclear targeting sequence. In some embodiments, the post transcriptional regulatory element and the nuclear targeting sequence comprise a sequence of SEQ ID NO: 12.

Aspects disclosed herein provide a nucleic acid comprising a nucleic acid sequence having at least 80% sequence identity to any one of SEQ ID NO: 4-5. In some embodiments, the nucleic acid has at least 81, 82, 83, 84, 85, 86, 87, 88, 89, 90, 91, 92, 93, 94, 95, 96, 97, 98, or 99% sequence identity to any one of SEQ ID NO: 4-5. In some embodiments, the nucleic acid which comprises the muscle specific promoter sequence facilitates expression of a transgene coupled to the promoter for at least 30, 60, 90, 120, 150, 180, 210, 240, 270, or 275 days. In some embodiments, a nucleic acid delivery vector comprises the muscle specific promoter sequence facilitates expression of a transgene coupled to the promoter for at least 30, 60, 90, 120, 150, 180, 210, 240, 270, or 275 days. In some embodiments, the nucleic acid is a muscle tissue specific promoter sequence. In some embodiments, the nucleic acid enhances expression of a therapeutic transgene in a muscle cell. In some embodiments, the muscle cell comprises a myocyte, a cardiomyocyte, or a smooth muscle cell. Aspects disclosed herein provide a nucleic acid delivery vector comprising the nucleic acid of any one of embodiments disclosed. Aspects disclosed herein provide a use of the nucleic acid of any one of embodiments disclosed in a method of treating a subject having a dystrophinopathy disorder requiring a dystrophin gene therapy or a dystrophin protein replacement therapy. Aspects disclosed herein provide a pharmaceutical composition comprising the nucleic acid of any one of embodiments disclosed. Aspects disclosed herein provide a use of the pharmaceutical composition of embodiments disclosed in a method of treating a subject having a dystrophinopathy disorder requiring a dystrophin gene therapy or a dystrophin protein replacement therapy. Aspects disclosed herein provide a cell comprising the nucleic acid of any one of embodiments disclosed. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte.

Aspects disclosed herein provide a nucleic acid comprising a sequence which has at least 80% sequence identity to any one of SEQ ID NO: 16-17. Aspects disclosed herein provide a nucleic acid comprising a sequence of SEQ ID NO: 16-17.

In addition to challenges associated with aspects of nucleic acid vector design outside of the transgene coding sequence, modifying the nucleic acid coding sequence of therapeutic transgenes can improve transgene expression even without modifying the amino acids coded by the nucleic acid coding sequence. Certain nucleic acid coding sequence, even those which are native to the human genome, can present challenges with inducing proper gene expression and protein synthesis in human gene therapies. For example, different organisms may prefer specific codons for the same amino acid, and non-preferred codons may result in inefficient protein production in certain organisms (e.g., mammalian organisms). In some cases, codon optimization can adjust codons to match the preferences of the host organism, improving translation efficiency. In other cases, mRNA can form secondary structures such hairpins or loops, which can block ribosomal translation of the mRNA into protein. Even in the absence of the formation of secondary structures, the sequence of the mRNA itself with too many repetitive sequences can trigger immune response to the transcribed mRNA. In other cases, the lack of optimized codons around the ribosomal binding site can reduce the initiation of translation by the ribosome.

Disclosed herein are optimized coding sequences of human dystrophin protein s which provide a beneficial technical effect of increasing expression of the dystrophin in the subject. In some embodiments, the nucleic acid encoding the dystrophin protein comprises the sequence of any one of SEQ ID NO: 2-3. In some embodiments, the nucleic acid encoding the dystrophin protein comprises the sequence of any one of SEQ ID NO: 6-7. In some embodiments, the cell is a myocyte. In some embodiments, the muscle cell comprises a skeletal muscle myocyte, a cardiomyocyte, or a smooth muscle cell.

Aspects disclosed herein provide a nucleic acid sequence comprising a sequence having at least 80% sequence identity to any one of SEQ ID NO: 2-3. In some embodiments, the nucleic acid sequence has at least 81, 82, 83, 84, 85, 86, 87, 88, 89, 90, 91, 92, 93, 94, 95, 96, 97, 98, or 99% sequence identity to any one of SEQ ID NO: 2-3. In some embodiments, the nucleic acid sequence is any one of SEQ ID NO: 2-3. In some embodiments, the nucleic acid sequence encodes a dystrophin protein. In some embodiments, the nucleic acid sequence encodes a full length dystrophin protein. In some embodiments, the nucleic acid sequence encodes a dystrophin protein at least 3500 amino acids in length. Aspects disclosed herein provide a nucleic acid delivery vector comprising the nucleic acid of embodiments disclosed herein. Aspects disclosed herein provide a use of the nucleic acids of any one of embodiments disclosed herein in a method of treating a subject having a dystrophinopathy disorder requiring a dystrophin gene therapy or a dystrophin protein replacement therapy. Aspects disclosed herein provide a pharmaceutical composition comprising the nucleic acid of embodiments disclosed herein. Aspects disclosed herein provide a use of the pharmaceutical composition of embodiments disclosed herein in a method of treating a subject having a dystrophinopathy disorder requiring a dystrophin gene therapy or a dystrophin protein replacement therapy. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte. Aspects disclosed herein provide a cell comprising the nucleic acid of embodiments disclosed herein.

In some embodiments, the muscle cell is located within a muscle tissue of the subject, the method further comprising one or more of: applying heat to the muscle tissue, or increasing a blood flow rate in the muscle tissue. In some embodiments, heat is applied to the muscle tissue using a transcutaneous compress at a temperature of at least 37 C. In some embodiments, heat is applied to the muscle tissue using ultrasound energy. In some embodiments, the muscle tissue is located in a heart or a diaphragm of the subject. In some cases, applying heat to the muscle tissue and/or increasing a blood flow rate provides a beneficial technical effect of increasing delivery of the nucleic acid encoding the functional dystrophin protein to the cell, and resulting expression of the functional dystrophin protein.

Aspects disclosed herein provide a nucleic acid comprising a promoter sequence having at least 90% sequence identity to any one of SEQ ID NOS: 37-40. In some embodiments, the promoter sequence comprises at least 91, 92, 93, 94, 95, 96, 97, 98, or 99% sequence identity to any one of SEQ ID NOS: 37-40. In some embodiments, the promoter sequence comprises any one of SEQ ID NOS: 37-40. In some embodiments, the promoter sequence is configured to increase expression of a nucleic acid encoding a therapeutic protein operably coupled to the promoter sequence. In some embodiments, the promoter sequence is configured to increase expression of a nucleic acid encoding a therapeutic protein operably coupled to the promoter sequence in multiple cell types. In some embodiments, the promoter sequence is configured to increase expression of the nucleic acid encoding the therapeutic protein in a muscle cell. In some embodiments, the promoter sequence is configured to increase expression of the nucleic acid encoding the therapeutic protein in a kidney cell. In some embodiments, the promoter sequence is configured to increase expression of the nucleic acid encoding the therapeutic protein in a liver cell. In some embodiments, the liver cell is a hepatocyte, or a LSEC. In some embodiments, the promoter sequence is configured to increase expression of the nucleic acid encoding the therapeutic protein in a myocyte. Aspects disclosed herein provide nucleic acid delivery vector comprising the nucleic acid of any one of the present embodiments disclosed herein. Aspects disclosed herein provide pharmaceutical composition comprising the nucleic acid of any one of the present embodiments disclosed herein Aspects disclosed herein provide cell comprising the nucleic acid of any one of the present embodiments disclosed herein. Aspects disclosed herein provide method of treating a subject comprising administering the nucleic acid of any one of the present embodiments disclosed herein. In some embodiments, the method further includes administering ultrasound to the subject. In some embodiments, the method further includes administering a sonoactive agent to the subject. In some embodiments, the method further includes administering a lipid nanoparticle or a polymer nanoparticle to the subject. In some embodiments, the nucleic acid is contained within a viral vector.

Aspects disclosed herein provide a nucleic acid comprising a nucleic acid sequence having at least 90% sequence identity to SEQ ID NO: 34. In some embodiments, the nucleic acid sequence has at least 91% sequence identity to SEQ ID NO: 34. In some embodiments, the nucleic acid sequence has at least 92% sequence identity to SEQ ID NO: 34. In some embodiments, the nucleic acid sequence has at least 93% sequence identity to SEQ ID NO: 34. In some embodiments, the nucleic acid sequence has at least 95% sequence identity to SEQ ID NO: 34. In some embodiments, the nucleic acid sequence has at least 97% sequence identity to SEQ ID NO: 34. In some embodiments, the nucleic acid sequence has at least 99% sequence identity to SEQ ID NO: 34. In some embodiments, the nucleic acid sequence is SEQ ID NO: 34. In some embodiments, the nucleic acid sequence encodes a dystrophin protein. In some embodiments, the nucleic acid sequence encodes a full length dystrophin protein. In some embodiments, the nucleic acid sequence encodes a dystrophin protein at least 3500 amino acids in length. In some embodiments, the nucleic acid sequence encodes a protein having at least 90% sequence identity to SEQ ID NO: 1. In some embodiments, the nucleic acid sequence comprises a muscle tissue specific promoter sequence. In some embodiments, the muscle tissue specific promoter sequence enhances expression of a dystrophin protein in a muscle cell. In some embodiments, the muscle cell comprises a myocyte, a cardiomyocyte, or a smooth muscle cell. In some embodiments, the nucleic acid sequence is an expression cassette for a dystrophin protein. In some embodiments, the nucleic acid sequence is an expression cassette for a dystrophin protein optimized for expression of the dystrophin protein in mammalian cells. In some embodiments, the nucleic acid sequence is an expression cassette for a dystrophin protein optimized for expression of the dystrophin protein in myocytes. In some embodiments, the nucleic acid sequence is comprised within a nucleic acid delivery vector having a sequence of SEQ ID NO: 17. In some cases, the expression cassette of SEQ ID NO: 34 provides a beneficial technical effect of improving delivery and expression of a dystrophin transgene encoded by the nucleic acid delivery vector in muscle cells of a subject.

Aspects disclosed herein provide a nucleic acid comprising a nucleic acid sequence having at least 90% sequence identity to SEQ ID NO: 4. In some embodiments, the nucleic acid sequence has at least 91% sequence identity to SEQ ID NO: 4. In some embodiments, the nucleic acid sequence has at least 92% sequence identity to SEQ ID NO: 4. In some embodiments, the nucleic acid sequence has at least 93% sequence identity to SEQ ID NO: 4. In some embodiments, the nucleic acid sequence has at least 94% sequence identity to SEQ ID NO: 4. In some embodiments, the nucleic acid sequence has at least 95% sequence identity to SEQ ID NO: 4. In some embodiments, the nucleic acid sequence has at least 96% sequence identity to SEQ ID NO: 4. In some embodiments, the nucleic acid sequence has at least 97% sequence identity to SEQ ID NO: 4. In some embodiments, the nucleic acid sequence has at least 98% sequence identity to SEQ ID NO: 4. In some embodiments, the nucleic acid sequence has at least 99% sequence identity to SEQ ID NO: 4. In some embodiments, the nucleic acid sequence is a muscle tissue specific promoter sequence. In some embodiments, the nucleic acid sequence enhances expression of a therapeutic transgene in a muscle cell. In some embodiments, the muscle cell comprises a myocyte, a cardiomyocyte, or a smooth muscle cell. In some embodiments, the nucleic acid sequence facilitates expression of a transgene coupled to the promoter for at least 30, 60, 90, 120, 150, 180, 210, 240, 270, or 275 days. In some embodiments, the nucleic acid sequence is comprised within a nucleic acid delivery vector and coupled to a nucleic acid coding sequence encoding a therapeutic transgene, wherein the therapeutic transgene encodes a protein having at least 90% sequence identity to SEQ ID NO: 1. Aspects disclosed herein provide a nucleic acid delivery vector comprising the nucleic acid which is a muscle tissue specific promoter sequence having at least 90% sequence identity to SEQ ID NO: 4. Aspects disclosed herein provide a pharmaceutical composition comprising the nucleic acid which is a muscle tissue specific promoter sequence having at least 90% sequence identity to SEQ ID NO: 4. Aspects disclosed herein provide a cell comprising the nucleic acid which is a muscle tissue specific promoter sequence having at least 90% sequence identity to SEQ ID NO: 4. Aspects disclosed herein provide a kit comprising: 1) a sonoactive agent; and 2) means for expressing a therapeutic transgene in a subject. Aspects disclosed herein provide a kit comprising: 1) a sonoactive agent; and 2) the nucleic acid which is a muscle tissue specific promoter sequence having at least 90% sequence identity to SEQ ID NO: 4. In some cases, the muscle specific promoter sequence of SEQ ID NO: 4 provides a beneficial technical effect of improving delivery and expression of a therapeutic transgene coupled to the muscle specific promoter sequence in muscle cells of a subject.

In some embodiments, administering the ultrasound acoustic energy comprises applying a first ultrasound energy at a MI of up to 0.4, and a first second ultrasound energy at a MI of greater than 0.4 and up to 3.0. In some embodiments, administering the ultrasound acoustic energy comprises applying a first ultrasound energy at a MI of up to 0.4, and a first second ultrasound energy at a MI of 0.8-1.9. In some embodiments, administering the ultrasound acoustic energy comprises applying an acoustic radiation force to the subject. In some embodiments, the ultrasound energy is applied at an intensity (ISPTA) of at least 200 mW/cm². In some embodiments, the ultrasound energy displaces a muscle tissue of the subject by at least 0.1 mm. In some embodiments, the muscle tissue is displaced by at least 0.1, 0.2, 0.3, 0.4, 0.5, 0.6, 0.7, 0.8, 0.9, or 1 mm. In some embodiments, the ultrasound energy is applied at a pulse length of at least 20 microseconds. In some embodiments, the cell or the myocyte is in a muscle tissue of the subject, wherein administering the sonoactive agent to the subject comprises distributing the sonoactive agent throughout the capillaries of the muscle tissue. In some embodiments, applying the ultrasound energy comprises applying an acoustic radiation force to the muscle tissue In some embodiments, applying the acoustic radiation force comprises displacing the muscle tissue In some embodiments, applying the acoustic radiation force comprises displacing a volume of blood in the capillaries of the muscle tissue In some embodiments, displacing the volume of blood in the capillaries of the muscle tissue transmits a pressure wave throughout a vascular system of the muscle tissue, thereby disrupting the sonoactive agent. In some embodiments, the pressure wave does and/or the ultrasound energy applying the acoustic radiation force does not induce vibrational cavitation of the sonoactive agent. In some embodiments, the sonoactive agent comprises a shell filled with a gas. In some embodiments, the shell comprises a protein stabilized shell or a lipid stabilized shell. In some embodiments, the gas comprises a perfluorinated case. In some embodiments, the sonoactive agent comprises microbubbles. In some embodiments, the sonoactive agent comprises nanobubbles. In some embodiments, the subject has a mutation in a gene encoding a dystrophin protein in a genome of the subject. In some embodiments, the subject has a dystrophinopathy disorder. In some embodiments, the dystrophinopathy disorder comprises Duchenne muscular dystrophy (DMD), Becker muscular dystrophy (BMD), or DMD-associated dilated cardiomyopathy. In some embodiments, the dystrophinopathy disorder comprises Duchenne muscular dystrophy (DMD), and wherein the cell is a skeletal muscle myocyte. In some embodiments, the dystrophinopathy disorder comprises Becker muscular dystrophy (BMD), and wherein the cell is a skeletal muscle myocyte. In some embodiments, the dystrophinopathy disorder comprises DMD-associated dilated cardiomyopathy, and wherein the cell is a cardiac myocyte. In some embodiments, administering the nucleic acid, administering the sonoactive agent, and applying the ultrasound energy induces expression of the dystrophin protein thereby treating the subject having the dystrophinopathy disorder. In some embodiments, the method reduces or ameliorates a symptom of the dystrophinopathy disorder in the subject. In some embodiments, the method further includes administering a second dose of the nucleic acid encoding the dystrophin protein, the sonoactive agent, and the ultrasound energy to the subject. In some embodiments, the subject is a mammalian subject. In some embodiments, the subject is a human subject.

Aspects disclosed herein provide a nucleic acid comprising a promoter sequence having at least 90% sequence identity to SEQ ID NO: 18. In some embodiments, the promoter sequence has at least 91, 92, 93, 94, 95, 96, 97, 98, or 99% sequence identity to SEQ ID NO: 18. In some embodiments, the promoter sequence is SEQ ID NO: 18.

Ultrasound refers to the application of electromagnetic energy in the range of greater than 20 kHz up to several gigahertz. Ultrasound is used in many different fields, most commonly in the field of diagnostics and medical imaging for producing images of tissue within the human body. Ultrasound energy can be generated at various frequencies within the 20 kHz up to several gigahertz range, most commonly within the range of about 1 to 10 megahertz when used for diagnostic imaging purposes. Ultrasound is commonly applied using ultrasonic transducers comprising one or more piezoelectric crystals which convert electrical energy into acoustic energy. In addition to imaging applications, ultrasound can also be used for a variety of other diagnostic and therapeutic applications, including determination of tissue elasticity and fibrosis, focused destruction of tissue using ultrasound ablation, and for the delivery of exogenous payloads (e.g., nucleic acids and therapeutic agents) to a cell. Sonoporation refers to the delivery of therapeutic agents, for example nucleic acids, using ultrasound and/or sonoactive microstructures to a cell. Provided in certain embodiments herein are methods of delivering a nucleic acid construct into a target cell or tissue (e.g., of a subject) by applying ultrasound energy to a cell, tissue, or organ (e.g., with sonoporation).

B mode ultrasound imaging refers to brightness mode imaging, in which ultrasonic waves are reflected from the tissue of a subject back to the ultrasound probe, and displayed on a 2 dimensional objects that are closer to the ultrasound transducer appear brighter, and objects which are farther away from the ultrasound transducer appear darker. B mode ultrasound imaging generally will focus ultrasound energy emitted from a plurality of ultrasound arrays comprising piezoelectric crystals into a focused ultrasound beam which penetrates into the tissue about a vertical axis which is perpendicular to the surface of the ultrasound probe. The focused ultrasound beam reflects off the tissue and back towards the ultrasound transducer, forming a scan line in an ultrasound image. By moving the ultrasound transducer about a surface of tissue, an image of the underlying tissue can be generated using a B mode ultrasound image. B mode ultrasound imaging is the most common form of ultrasound used in the United States for medical imaging, and is what is commonly referred to as diagnostic or imaging ultrasound.

Plane wave imaging refers to an ultrasound imaging technique in which a plurality of ultrasound arrays comprising piezoelectric crystals in an ultrasound transducer are simultaneously fired without directing ultrasound energy into a focused ultrasound beam, and which instead direct a large unfocused sheet or wave of ultrasound energy into a medium or tissue underlying an ultrasound probe. The primary difference between plane wave ultrasound imaging and B mode ultrasound imaging is the number of transducer arrays which are fired. Plain wave imaging typically will fire all arrays within an ultrasound transducer Producing a much larger and less focused wave of ultrasonic energy, while B mode imaging will typically only fire a subset of arrays which focused the ultrasound into a beam producing what is commonly referred to as a scan line. In plane wave imaging, the acoustic radiation pressure is almost uniform over the entire field of view, and lower peak and negative pressures are typically experienced as compared to traditional beam mode focused ultrasound beam imaging.

Acoustic radiation force refers to a static or transient force applied by an acoustic wave on the propagation medium or to an object in the path of the acoustic wave. Acoustic radiation forces can be applied using an ultrasound transducer when applying ultrasound energy to a surface of a tissue or a propagation medium with sufficient ultrasound intensity. When applying a sufficient acoustic radiation force to a propagation medium or a tissue, the propagation medium or tissue under lying the ultrasound probe applying the acoustic radiation force may be displaced.

Shear waves, or secondary waves refer to transversely oriented waves which occur in elastic medium that is subjected to a periodic shear. Shear refers to a change in shape without a change of volume of a layer of a propagation medium or tissue produced by a pair of equal forces acting in opposite directions about two faces of the layer or the propagation medium. Shear waves are a type of elastic wave which move through the body of an object or a propagation medium. In an elastic medium, the layer or the tissue will resume its original shape following application of the shear force, adjacent layers will undergo subsequent shear, and the movement of particles within the medium or tissue will be propagated as a shear wave throughout the propagation medium or tissue. In an elastic medium, shear waves can be produced as a secondary wave following a compressional wave which is transmitted in the propagation medium or tissue. Ultrasound applying an acoustic radiation force can apply a compressional wave to a tissue, which can result in application of shear waves to a tissue when applied with sufficient intensity, at regular intervals, for sufficient periods of time to induce a regular shear in layers of a tissue. A compressional wave displaces tissue in a direction parallel to the propagation of the compressional waves. An ultrasound transducer can induce a compressional wave in a tissue which propagates from the ultrasound transducer about a vector normal to a surface of the ultrasound transducer. In some embodiments, applying the focused acoustic radiation force to the tissue comprises generating a compressional wave in the tissue. As an elastic tissue recovers from displacement due to a compressional wave, shear waves or secondary waves can be generated. In a shear wave, the direction of particle motion is parallel to the direction of propagation of the compressional wave, and the direction of propagation of the shear wave is normal to the direction of propagation of the compressional wave. The direction of particle motion in a shear wave is also normal to the direction of shear wave propagation in an elastic medium. Further, a compressional wave may be followed by a rarefaction wave which is a negative acoustic force in the tissue.

Shear wave elastography refers to a diagnostic technique using ultrasound to determine the elastic modulus of tissue, which is indicative of its fibrotic quality. Diseased tissue with certain fibrotic conditions will result in a significantly reduced elastic modulus of the tissue, as compared to a healthy tissue which is reasonably elastic as compared to diseased tissue in a fibrotic state. Shear wave elastography is a diagnostic imaging technique which uses a combination of acoustic radiation force, plane wave imaging, and B mode imaging to provide a clinician with information as to the fibrotic quality of a tissue. Shear wave elastography applies an acoustic radiation force to displace the tissue underlying an ultrasound probe with a compressional wave, thereby generating shear waves in the tissue, applies a plane wave ultrasound to the tissue to monitor the propagation of the shear waves throughout the tissue thereby calculating the elastic modulus, and overlays this data atop a standard B mode ultrasound image in order to provide a visual representation of tissue stiffness. Using ultrasound machines configured to perform shear wave elastography, an acoustic radiation force to displace the tissue underlying an ultrasound probe with a compressional wave, thereby generating shear waves in the tissue, can be applied. In some cases, a standard B mode ultrasound image and/or plane wave in order to calculate or provide a visual representation of tissue stiffness may not be performed.

Provided in certain embodiments herein are methods of delivering a nucleic acid construct into a target cell or tissue (e.g., of a subject) by applying ultrasound energy to a cell, tissue, or organ. Provided in certain embodiments herein are methods of delivering a nucleic acid construct into a target cell or tissue (e.g., of a subject) by delivering an exogenous payload to the subject; and applying a focused acoustic radiation force (ARF) to the subject, thereby generating shear waves in the tissue of the subject, wherein the focused acoustic radiation force enhances delivery of the exogenous payload to the cell in the tissue of an organ of the subject. Disclosed herein are methods of sonoporation in which an exogenous payload is delivered to a cell in a tissue of a subject using a focused acoustic radiation force applied using ultrasound. Aspects of the sonoporation methods disclosed herein may also include inducing displacing the tissue of the subject with the acoustic radiation force to induce propagation of shear waves throughout the tissue of the subject thereby enhancing delivery of a nucleic acid payload to a cell. Disclosed herein are methods of sonoporation in which an exogenous payload is delivered to a cell in a tissue of a subject using a focused acoustic radiation force applied using ultrasound. Aspects of the sonoporation methods disclosed herein may also include inducing displacing the tissue of the subject with the acoustic radiation force to induce propagation of shear waves throughout the tissue of the subject thereby enhancing delivery of a nucleic acid payload to a cell. The method may further include applying the acoustic radiation force to induce propagation of shear waves throughout the tissue in combination with other secondary ultrasound energies such as plane wave ultrasound or focused beam ultrasound in which the secondary ultrasound energy moves sonoactive microstructures toward an endothelial border of a tissue comprising the cell, while applying the focused acoustic radiation force during shear wave propagation induces inertial cavitation of sonoactive microstructures at the endothelial border of the tissue comprising the cell, thereby enhancing delivery of the therapeutic payload to a cell, and, in cases of a nucleic acid payload, resulting gene expression.

In some embodiments, the focused acoustic radiation force is applied using an ultrasound probe applying ultrasound energy to the tissue. In some embodiments, the ARF displaces the tissue of the subject. In some embodiments, the shear waves displace the tissue of the subject. In some embodiments, a tissue displacement is at least 0.001 mm. In some embodiments, a tissue displacement ranges from at least 0.001 mm to about 5 mm. In some embodiments, a tissue displacement ranges from 0.01 mm to about 1 mm.

In some embodiments, the focused acoustic radiation force is applied using an ultrasound probe applying ultrasound energy to the tissue. In some embodiments, the ultrasound energy is applied at a mechanical index greater than 0.4. In some embodiments, the ultrasound energy is applied at a mechanical index of about 1.4. In some embodiments, the ultrasound energy is applied at a mechanical index of at least 1.3. In some embodiments, the ultrasound energy is applied at a mechanical index of greater than 0.4 up to about 3.0. In some embodiments, the ultrasound energy is applied at a frequency of about 0.1 MHz to about 10 MHz. In some embodiments, the ultrasound energy is applied at a frequency of about 2.5 MHz. In some embodiments, the applying ultrasound energy to the tissue comprises applying the ultrasound energy at an ultrasound intensity of at least 100 mW/cm². In some embodiments, the applying ultrasound energy to the tissue comprises applying the ultrasound energy at an ultrasound intensity of about 100 mW/cm² to about 10,000 mW/cm². In some embodiments, the applying ultrasound energy to the tissue comprises applying the ultrasound energy at an ultrasound intensity of about 100 mW/cm² to about 5,000 mW/cm². In some embodiments, the applying ultrasound energy to the tissue comprises applying the ultrasound energy at an ultrasound intensity of about 100 mW/cm² to about 500 mW/cm². In some embodiments, the applying ultrasound energy to the tissue comprises applying the ultrasound energy at an ultrasound intensity of about 110 mW/cm² to about 200 mW/cm². In some embodiments, the applying ultrasound energy to the tissue comprises applying the ultrasound energy at an ultrasound intensity of about 188 mW/cm². In some embodiments, the ultrasound intensity is a spatial-peak temporal average intensity. In some embodiments, the spatial-peak temporal average intensity is calculated in a focal region of the tissue. In some embodiments, the focused acoustic radiation force is applied in two or more pulses, with an interval between each of the two or more pulses. In some embodiments, a plane wave ultrasound is applied to the tissue during the interval. In some embodiments, the one or more pulses are up to 500 microseconds. In some embodiments, the one or more pulses are at least 100 microseconds. In some embodiments, the one or more pulses are about 100 microseconds to about 500 microseconds. In some embodiments, the interval is up to 500 milliseconds. In some embodiments, the interval is up to 100, 500, 1000, 1500, 2000, 2500, 3000, 4000, or 5000 milliseconds. In some embodiments, the interval is from about 100 milliseconds to about 5000 milliseconds. In some embodiments, the applying the focused acoustic radiation force is performed in one or more sequences, wherein a sequence comprises two or more pulses and interval(s) therebetween. In some embodiments, a time between application of the one or more sequences is at least 5, 10, 20, 30, 60, 120, 180, 240, 300, 360, 420, 480, 540, or 600 seconds. In some embodiments, a time between application of the one or more sequences ranges from about 5 to about 300 seconds. In some embodiments, a time between application of the one or more sequences ranges from about 10 to about 60 seconds. In some embodiments, the focused acoustic radiation force is applied for at least 10, 20, 30, 60, 120, 180, 240, 300, 360, 420, 480, 540, or 600 seconds. In some embodiments, applying ultrasound energy to the tissue comprises applying the ultrasound energy at a focal depth of up to 10, 8, 6, or 4 cm from the ultrasound transducer. In some embodiments, applying ultrasound energy to the tissue comprises applying the ultrasound energy at a focal depth of about 1 to about 10 cm from the ultrasound transducer. In some embodiments, applying ultrasound energy to the tissue comprises applying the ultrasound energy at a focal depth of about 4 to about 10 cm from the ultrasound transducer. In some embodiments, applying ultrasound energy to the tissue comprises applying the ultrasound energy at a focal depth of about 4 cm from the ultrasound transducer. In some embodiments, applying ultrasound energy to the tissue comprises applying the ultrasound energy at a focal depth of about 6 cm from the ultrasound transducer.

In some embodiments, applying the focused wave ultrasound results in moving the sonoactive microstructures towards an endothelial border of the tissue comprising the cell. In some embodiments, the shear waves induce inertial cavitation the sonoactive microstructures at an endothelial border of the tissue comprising the cell, thereby enhancing delivery of the nucleic acid to the cell. In some embodiments, the focused acoustic radiation force increases internalization of the exogenous payload in the cell. In some embodiments, the shear waves increase internalization of the exogenous payload in the cell. In some embodiments, inducing inertial cavitation the sonoactive microstructures increases internalization of the exogenous payload in the cell.

In some embodiments, a process provided herein provides sonoporation at two or more different ultrasonic acoustic energies (e.g., a first and second ultrasound energy having a first and second MI, respectively). In certain embodiments, a process provided herein provides a process wherein an ultrasound energy is continuously applied (e.g., ultrasound energy transitions from the first ultrasound energy to the second ultrasound energy, without a period of no ultrasound energy being applied). In certain embodiments, a transitory (e.g., third, fourth, etc.) ultrasound energy is applied between application of the first and second ultrasonic acoustic energies. Provided in certain embodiments herein are methods of delivering a nucleic acid construct into a target cell or tissue (e.g., of a subject) by applying a first ultrasound energy to a cell, tissue, or organ, and applying a second ultrasound energy to the cell, tissue, or organ. In some embodiments herein are methods for transfecting a nucleic acid construct into a target cell or tissue by applying a first ultrasound energy having a first mechanical index (MI) and applying a second ultrasound energy having a second mechanical index (MI). The present disclosure provides methods for enhancing transfection of a nucleic acid construct into the target cell or tissue by applying alternating ultrasound energy, the alternating acoustic energy alternating between a first mechanical index (MI) and a second MI. Application of ultrasound energy can be repeated several times during sonoporation, such as to increase the efficiency of nucleic acid construct transfection and/or delivery.

In some embodiments, the sonoporation treatment comprises applying an ultrasound energy to the target cell (e.g., of a tissue or organ of the subject) (e.g., the ultrasound energy having a mechanical index (MI)). In some embodiments, applying an ultrasound energy to the target cell comprises applying a first ultrasound energy to the target cell and applying a second ultrasound energy to the target cell. In some embodiments, the (e.g., first or second) ultrasound energy has a first mechanical index (MI). In certain embodiments, (e.g., the other of the first or second) ultrasonic energy has a second mechanical index (MI). In some embodiments, the (e.g., first or second) MI is less than 0.4. In certain embodiments, the (e.g., the other of the first or second) MI is greater than 0.4 (e.g., and less than 2.0). In some embodiments, a first ultrasound energy and a second ultrasound energy are applied sequentially in a repeated manner. In some embodiments, a first MI is a Low MI (e.g., less than 0.4). In certain embodiments, a second MI is a High MI (e.g., 0.4 or greater). In some embodiments, a first MI is a Low MI (e.g., less than 0.4) and a second MI is a High MI (e.g., 0.4 or greater). In some embodiments, a second MI is a Low MI (e.g., less than 0.4). In certain embodiments, a first MI is a High MI (e.g., 0.4 or greater). In given embodiments, a second MI is a Low MI (e.g., less than 0.4) and a first MI is a High MI (e.g., 0.4 or greater). In some embodiments, a Low MI is less than 0.3. In given embodiments, a Low MI is less than 0.2. In more given embodiments, a Low MI is less than 0.1. In still more given embodiments, a Low MI is about 0.09. In still more given embodiments, a Low MI is about 0.04. In still more given embodiments, a Low MI is about 0.03. In some embodiments, a High MI is greater than 0.5. In given embodiments, a High MI is 0.5 to 2.0 or is between 0.5 and 2.0. In more given embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is greater than 0.4. In some embodiments, a High MI is greater than 0.5. In more given embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is 2.2. In some embodiments, a High MI is 2.5. In some embodiments, a High MI is 2.8. In some embodiments, a High MI is 3.0. In certain embodiments, any process provided herein (e.g., a sonoporation treatment) comprises administering of a continuous ultrasound energy (which may have varying energy level) that alternates (e.g., in identical, similar, or variable periods) between Low MI and High MI. In some embodiments, a low MI (e.g., less than 0.1) (e.g., first) ultrasound energy (also referred to herein as a Low MI) is administered to the subject, and a set number pulses (e.g., of less than 30 seconds) of High MI (e.g., second) ultrasound energy (also referred to herein as a High MI) is administered to the subject. In some embodiments, a process provided herein comprises administration of a plurality of pulses of high MI (e.g., second) ultrasound energy, e.g., during an otherwise continuous administration of a low MI (e.g., first) ultrasound energy. In given embodiments, the number of High MI pulses is about 4 or more, 8 or more, 12, or more, 16 or more, 18 or more, 25 or more, such as up to about 30, or an unlimited number of pulses. In given embodiments, the number of High MI pulses is 6-30. In still more given embodiments, the number of High MI pulses is between 8, 9, 12, 15, 18, 27, 36, 45, or any number therebetween. In some embodiments, at least 8, 9, 12, 15, or 18 high MI pulses are administered to the subject in between applications of low MI ultrasound energy. In still more given embodiments, the number of High MI pulses applied is at least 8, 9, 12, 15, 18, 27, 36, or 45. In still more given embodiments, the number of High MI pulses applied in a given sequence of high MI ultrasound energy is at least 8, 9, 12, 15, 18, 27, 36, or 45.

In certain embodiments, the first (either High MI or Low MI) ultrasound energy is applied before or after administration of any other agent, such as the nucleic acid and/or sonoactive structure. In some embodiments, the first ultrasound energy is applied after administration of the sonoactive structure to the subject. In certain embodiments, the first ultrasound energy is applied after administration of the nucleic acid to the subject. In some embodiments, the first ultrasound energy is applied after administration of both the nucleic acid and the sonoactive structure(s).

In some embodiments, high MI ultrasound energy is administered in a pulse. In given embodiments, a pulse length is any suitable length, such as less than 30 seconds. In more given embodiments, a pulse length is less than 15 seconds. In still more given embodiments, a pulse length is less than 10 seconds. In yet more given embodiments, a pulse length is less than 5 seconds. In more given embodiments, a pulse length is less than 2 seconds. In still more given embodiments, a pulse length is less than 1 second and/or may be greater than or equal to 1 microsecond. In some embodiments, a pulse length ranges from 100 to 300 microseconds. In some embodiments, a pulse length is up to about 200 microseconds. In some embodiments, a pulse length is up to about 500 microseconds. In some embodiments, a pulse length ranges from 1 to 500 microseconds.

In various embodiments, a High MI ultrasound energy is provided first temporally (e.g., first in order). In other embodiments, a Low MI ultrasound energy is provided second temporally (e.g., second in order).

In some embodiments, any process provided herein further comprises administering (e.g., systemically administering, such as via infusion) a nucleic acid (e.g., any nucleic acid provided herein) to a subject (e.g., to whom the ultrasonic acoustic energies are applied).

In some embodiments, any process provided herein further comprises administering (e.g., systemically administering, such as via infusion) a sonoactive structure (e.g., any sonoactive structure or microbubble described herein) to a subject (e.g., to whom the ultrasonic acoustic energies are applied).

In certain embodiments, provided herein is a method of delivering a nucleic acid payload in a target cell (e.g., of a tissue or organ) of a subject, the method comprising: (a) administering to the subject a nucleic acid construct comprising the nucleic acid payload; (b) administering to the subject a plurality of sonoactive microstructures; and (c) administering an ultrasound energy, thereby delivering a sonoporation treatment.

In some embodiments, a sonoporation treatment (e.g., application of a first ultrasound energy, a second ultrasound energy, a single cycle of a first ultrasound energy and a second ultrasound energy, or series of cycles comprising a plurality of applications of a first ultrasound energy and a plurality of applications of a second acoustic energy) can last for a few seconds (e.g., 1-100 seconds) or more, such as up to a few minutes (e.g., 1-3 minutes). In given embodiments, a sonoporation treatment lasts for 1-30 seconds. In some given embodiments, a sonoporation treatment lasts for 5-100 seconds. In certain embodiments, a sonoporation treatment lasts for at least 1 minute (e.g., 1-30 minutes).

In some embodiments, the first ultrasound energy is administered within 60 minutes of administration of the nucleic acid and/or sonoactive structure(s). In given embodiments, the first ultrasound energy is administered within 30 minutes of administration of the nucleic acid and/or sonoactive structure(s). In more given embodiments, the first ultrasound energy is administered within 5 minutes of administration of the nucleic acid and/or sonoactive structure(s). In still more given embodiments, the first ultrasound energy is administered within 2 minutes of administration of the nucleic acid and/or sonoactive structure(s). In still more given embodiments, the first ultrasound energy may be applied simultaneously with administration of the nucleic acid and/or sonoactive structure(s).

In given embodiments, the first (e.g., High MI) ultrasound energy is applied immediately upon administration (e.g., infusion) or a period of time after administration (e.g., infusion) of the sonoactive structure(s) and/or nucleic acid.

In some embodiments, either the first or second ultrasound energy is an ultrasound energy (e.g., Low MI) that when applied to a cell, tissue, or organ of a subject results in stable cavitation (or stable vibrational cavitation) of the sonoactive structure and/or a change in the average diameter of the sonoactive structure(s), for example, due to inherent resonance properties of the microbubbles.

In certain embodiments, the first or second ultrasound energy is an ultrasound energy (e.g., High MI) that when applied to a cell, tissue, or organ of a subject results in inertial cavitation or the collapse of the sonoactive structures and/or disruption of cell membrane and/or vascular endothelial integrity.

In certain embodiments, either the first or second ultrasound energy is an ultrasound energy (e.g., Low MI) that when applied to a cell, tissue, or organ of a subject results in stable cavitation (or stable vibrational cavitation) and/or a change in the average diameter of the sonoactive structure(s), and the other of the first or second ultrasound energy is an ultrasound energy (e.g., High MI) that when applied to a cell, tissue, or organ of a subject results in inertial cavitation or the collapse of the sonoactive structures and/or disruption of cell membrane and/or vascular endothelial integrity.

In some instances, disruption of cell membrane allows target cells to become permeable to circulating agents such as nucleic acid constructs. In certain instances, such circulating agents can then enter the target cells, tissues or organs, such as in a more rapid manner (e.g., relative to either Low MI or High MI ultrasound energy application alone, or in the absence of ultrasound energy application).

In some embodiments, the methods herein comprise alternating the ultrasound energy applied between a first ultrasound energy having a first MI and a second ultrasound energy having a second MI. In some embodiments, applying alternating ultrasound energy administered to a subject between a first MI and a second MI is performed repeatedly over a number of times, such as to enhance gene transfection into the target cells, tissue or organ (e.g., relative to a similar process wherein a first and second ultrasound energy are not used and/or are not alternately applied and/or are not alternately applied repeatedly).

In some embodiments, the first MI ranges from about 0.05 to less than 0.4. In some embodiments, the first MI ranges from about 0.05 to about 0.3. In some embodiments, the first MI ranges from about 0.05 to less than 0.4. In some embodiments, the first MI ranges from about 0.09 to about 0.3.

In some embodiments, the second MI ranges from about 0.4 to about 3.0. In some embodiments, the second MI ranges from about 0.5 to about 3.0. In some embodiments, the second MI ranges from greater than 1.4 to about 3.0. In some embodiments, the second MI ranges from greater than 1.4 to about 3.0. In some embodiments, the second MI ranges from about 1.5 to about 3.0.

In some instances, the ultrasound energy applied at the second MI (e.g., high MI) is applied using a pulse. In some instances, a pulse comprises applying the ultrasound energy in a short pulse (e.g., microsecond length pulse). In some cases, the high MI is applied with the pulse, results in induces inertial cavitation and destruction of the sonoactive microstructure, resulting in the disruption of cell membrane and vascular endothelial integrity, transducing the nucleic acid payload to the cell. In some instances, the pulse is applied with a duration of about 1 μs to about 200 μs. In some instances, the pulse is applied with a duration of about 1 μs to about 200 μs or greater.

In some embodiments, applying the ultrasound energy at the second MI comprises applying the ultrasound energy at the second MI using a pulse. In some instances, the duration of the second MI applied ranges from 0.1 μs to about 200 μs. In some instances, the duration of the second MI applied ranges from 1 μs to about 200 μs or greater. In some embodiments, applying the ultrasound energy at the second MI comprises applying the ultrasound energy at the second MI using a pulse with a duration of about 1 μs to about 200 μs. In some embodiments, applying the ultrasound energy at the second MI comprises applying the ultrasound energy at the second MI using a pulse with a duration of up to 200 μs. In some embodiments, applying the ultrasound energy at the second MI comprises applying the ultrasound energy at the second MI using a pulse with a duration of about 1 μs to about 500 μs. In some embodiments, applying the ultrasound energy at the second MI comprises applying the ultrasound energy at the second MI using a pulse with a duration of up to 500 μs. In some embodiments, applying the ultrasound energy at the second MI comprises applying the ultrasound energy at the second MI using a pulse with a duration of about 2.3 μs. In some embodiments, applying the ultrasound energy at the second MI comprises applying the ultrasound energy at the second MI using a pulse with a duration of at least 2.3 μs. In some embodiments, applying the ultrasound energy at the second MI comprises applying the ultrasound energy at the second MI using a pulse with a duration ranging from 1-500 μs. In some embodiments, applying the ultrasound energy at the second MI comprises applying the ultrasound energy at the second MI using a pulse with a duration ranging from 0.1-500 μs.

In some embodiments, the ultrasound energy is applied using an ultrasound probe applying ultrasound energy to the tissue. In some embodiments, the acoustic radiation force is applied using an ultrasound probe applying ultrasound energy to the tissue. In some embodiments, the ultrasound probe comprises a plurality of piezoelectric elements configured to emit ultrasound energy. In some embodiments, portions of the plurality of piezoelectric elements are arranged in one or more arrays. In some embodiments, the ultrasound probe is a phased array transducer comprising a plurality of piezoelectric elements configured to emit ultrasound energy. In some embodiments, the ultrasound probe is a phased array ultrasound probe, a linear ultrasound probe, a curvilinear ultrasound probe, a convex array ultrasound probe, an endocavitary ultrasound probe, a 3D ultrasound probe, a 4D ultrasound probe, a Doppler ultrasound probe, or a color doppler ultrasound probe.

Sonoactive agent (also referred to as sonoactive microstructures acoustic microspheres or “microbubbles”) contemplated herein include, but are not limited to, those used as ultrasonic imaging contrast agents. In some embodiments, the sonoactive agent comprise a phospholipid stabilized microstructure. In some embodiments, the phospholipid stabilized microstructure comprises a high molecular wight gas core, or a perflutran core. Examples of sonoactive agent include, but are not limited to, OPTISON (GE Healthcare), Sonazoid (GE Healthcare), or DEFINITY and Definity RT (Lantheus Medical Imaging, Inc). In some embodiments, the sonoactive agent are LUMASON (Bracco) (sulfur hexafluoride lipid-type A microspheres). In some embodiments, the sonoactive agent are Sono Vue (sulfur hexafluoride microbubbles). In some embodiments, the sonoactive agent comprise a protein stabilized microstructure. In some embodiments, the sonoactive agent are Optison microbubbles.

The sonoactive agent can be administered prior to, after, or simultaneous (e.g., co-administered) with the administration of the nucleic acid (or cargo polynucleotide). In some embodiments, the nucleic acid and the sonoactive agent are coadministered. In some embodiments, the administering of the nucleic acid and the sonoactive agent occurs serially, concurrently, sequentially, or continuously. In some embodiments, the administering of the nucleic acid and the sonoactive agent occurs serially. In some embodiments, the administering of the nucleic acid and the sonoactive agent occurs concurrently. In some embodiments, the administering of the nucleic acid and the sonoactive agent occurs sequentially. In some embodiments, the administering of the nucleic acid and the sonoactive agent occurs continuously.

In some embodiments, the nucleic acid is administered at a dosage of about 0.5 mg/kg to about 500 mg/kg. In some embodiments, about 2×10 \hat{ }13 to about 3×10 \hat{ }13 copies of the nucleic acid are administered to the subject.

In some embodiments, the sonoactive microstructures are administered at a dosage of about 1-50 mL, for example 1 mL of Optison. The sonoactive microstructures may be administered at a concentration of about 5M to about 8M microstructures per mL. In some embodiments, the sonoactive microstructures are administered at a concentration of about 5×10\hat{ }8 to about 1.2×10 \hat{ }9 microstructures/mL, for example 1×10 \hat{ }9 of Definity RT. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10 \hat{ }9 microstructures/mL. In some embodiments, the sonoactive microstructures are administered at a concentration of about 5×10 \hat{ }8 to about 8×10 \hat{ }8 microstructures/mL.

In some embodiments, the nucleic acid and the sonoactive microstructures are mixed prior to being coadministered. In some instances, the sonoactive microstructures are mixed with the nucleic acids before administering to the subject. In some instances, the sonoactive microstructures are mixed with the nucleic acids along with additional buffers or agents such as saline or other biocompatible solutions with varying electrostatic charges and surface chemistries and ligands before administering to the subject. For example, Optison sonoactive microstructures can be mixed with a miniplasmid construct, e.g., a Nanoplasmid, comprising a promoter coupled to a transgene, e.g., APOE-Fluc, and saline, and administered together.

In some embodiments, the administering of the nucleic acid and the sonoactive agent is by intravenous administration or subcutaneous or intramuscular or intra-arterial or inter-osseus or direct organ puncture.

In some embodiments, after administering of the nucleic acid and sonoactive agent, the ultrasound acoustic energy is applied at the target cell, tissue, or organ.

Once the nucleic acids are inside the target cell, expression of the cargo polynucleotide is induced. In some embodiments, the cargo polynucleotide comprises luciferase.

In some embodiments, inducing expression of the cargo polynucleotide comprises inducing expressing within about 3 to about 12 hours of administering the pay load. In some embodiments, inducing expression of the cargo polynucleotide comprises inducing expressing within about 3 hours of administration. In some embodiments, inducing expression of the cargo polynucleotide comprises inducing expressing within about 6 hours of administration. In some embodiments, inducing expression of the cargo polynucleotide comprises inducing expressing within about 12 hours of administration.

Undesirable effects on living cells or tissues can occur due to ultrasound applications. In some embodiments, the present disclosure provides methods for improvement of gene transfection and not result in substantial DNA or cell damage in the target cells, tissues, or organs, using sonoporation by alternating ultrasonic acoustic energy between the first MI and the second MI. In some embodiments, the method does not result in substantial cellular damage to the target cell. In some embodiments, the method results in less than 1%, 5%, or 10% of target cells undergoing apoptosis.

Aspects disclosed herein provide a kit comprising: 1) a sonoactive agent; and 2) means for expressing a functional dystrophin protein in a subject. Aspects disclosed herein provide a kit comprising: 1) a sonoactive agent; and 2) a nucleic acid of any one of SEQ ID NO: 1-50. Aspects disclosed herein provide a kit comprising: 1) a sonoactive agent; and 2) the nucleic acid of any one of claim 98-106, or 111-116. In some embodiments, the kit further comprises instructions for administering ultrasound acoustic energy to a subject to facilitate expression of the functional dystrophin protein in a subject. In some embodiments, the kit further comprises instructions for administering ultrasound acoustic energy to a subject to facilitate expression of the nucleic acid in a subject. In some embodiments, the sonoactive agent comprises lipid-stabilized microstructures. In some embodiments, wherein the lipid-stabilized microstructures comprise a lipid stabilized shell surrounding a perfluorinated gas core. In some embodiments, the lipid stabilized shell comprises a monomolecular membrane of hydrogenated egg yolk phosphatidyl serine, wherein the perfluorinated gas core comprises perfluorobutane gas. In some embodiments, the sonoactive agent is a Sonazoid microbubble. In some embodiments, the sonoactive agent comprises protein stabilized microstructures. In some embodiments, the protein stabilized microstructures comprise an albumin stabilized shell surrounding a perfluorinated gas core. In some embodiments, the protein stabilized microstructures are Optison microbubbles.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

As used herein the term “sequence identity” refers to the percentage identity calculated as the matching residues divided by the total number of residues in the total alignment when performing a consensus alignment of two sequences which considers all residues in the query sequence under evaluation with gaps in the alignment scored as a mismatching residue, with the alignment determined by aligning the sequences using the Needleman-Wunsch alignment algorithm as implemented using the “Global Align” BLAST program available at https://blast.ncbi.nlm.nih.gov/Blast.cgi (with match/mismatch scores of 2,−3, a gap existence penalty of 5, and a gap extension penalty of 2) and comparing the aligned sequences. The number of exact matches divided by the total number of resides in the alignment (which corresponds with the number of nucleotides in the reference sequence plus any gaps in the reference sequence when aligned with the query sequence, including gaps or mismatches introduced at the beginning or the end of the sequence) is determined and expressed as a percentage. This is the percent sequence identity between the query sequence and the reference sequence (i.e., percent sequence identity=(#of exact matches)/(total #of residues in the query sequence)*100).

The terms “subject,” “individual,” or “patient” are often used interchangeably herein. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### II. EXEMPLARY EMBODIMENTS

Among the exemplary embodiments are:
1. A method of expressing a nucleic acid encoding a dystrophin protein in a cell of a subject, comprising: administering the nucleic acid encoding the dystrophin protein to the subject, administering a sonoactive agent to the subject, and applying ultrasound energy to the cell.
2. A method of expressing a functional dystrophin protein in a subject, comprising: administering an expression vector encoding the functional dystrophin protein or a functional fragment thereof to the subject such that at least 0.25% of cells in a population of cells of the subject uptake the expression vector.
3. A method of expressing a functional dystrophin protein in a subject, comprising: administering an expression vector encoding a functional dystrophin protein or a functional fragment thereof to the subject, thereby inducing uptake of the expression vector in a cell of a diaphragm of the subject.
4. A method of treating a dystrophinopathy disorder in a subject in need thereof, comprising: administering to the subject a nucleic acid encoding a functional dystrophin protein such that the functional dystrophin protein or a functional fragment thereof is expressed in a myocyte of the subject in an amount exceeding 0.5% of a dystrophin level in a wild type subject not having the dystrophinopathy disorder, thereby treating the dystrophinopathy disorder.
5. A method of treating a dystrophinopathy disorder in a subject in need thereof, comprising: administering to the subject a nucleic acid encoding a functional dystrophin protein, expressing the functional dystrophin protein or a functional fragment thereof in a myocyte of the subject about the cellular membrane of the myocyte, throughout the cytoplasm of the myocyte, or both about the about the cellular membrane and throughout the cytoplasm of the myocyte, thereby treating the dystrophinopathy disorder.
6. Use of (i) a nucleic acid encoding a functional dystrophin protein or a functional fragment thereof and (ii) a sonoactive agent in a method of treating a subject having a dystrophinopathy disorder requiring a dystrophin gene therapy or a dystrophin protein replacement therapy.
7. A method of treating a dystrophinopathy disorder in a subject in need thereof, comprising: administering to the subject a nucleic acid encoding a functional dystrophin protein, thereby delivering the nucleic acid to a myocyte in a muscle tissue of the subject (i) along a length of a muscle fiber in the muscle tissue and/or (ii) about the sarcolemma of a muscle fiber in the muscle tissue, thereby treating the dystrophinopathy disorder.
8. A method of delivering a nucleic acid encoding a functional dystrophin protein in a subject, comprising, comprising: administering to the subject the nucleic acid encoding the functional dystrophin protein, and transfecting the nucleic acid to a muscle tissue of the subject, wherein at least 5% of muscle fibers in the muscle tissue of the subject contain the nucleic acid.
9. A method of delivering a nucleic acid encoding a functional dystrophin protein in a muscle cell in a muscle tissue of a subject, comprising: administering the nucleic acid encoding the dystrophin protein to the subject wherein a coding sequence of the nucleic acid encoding the dystrophin protein is at least 10000 nucleotides; administering a sonoactive agent to the subject; and applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue thereby disrupting the sonoactive agent and delivering the nucleic acid encoding the dystrophin protein to at least 5% of muscle fibers in the muscle tissue.
10. The method of any one of embodiments 1-8, wherein the nucleic acid encodes a full length dystrophin protein.
11. The method of embodiment 10, wherein a coding sequence of the nucleic acid encoding the full length dystrophin protein is at least 5000, 6000, 7000, 8000, 9000, 10000, or 11000 nucleotides.
12. The method of embodiment 10, wherein a length the functional dystrophin protein is at least 1750, 2000, 2250, 2500, 2750, 3000, or 3250 amino acids.
13. The method of any one of embodiments 9-12, wherein the coding sequence of the nucleic acid encoding the full length dystrophin protein is contiguous.
14. The method of embodiment 1, wherein the cell is a myocyte, and wherein the dystrophin protein or a functional fragment thereof is expressed in the myocyte in an amount exceeding 0.5% of a dystrophin level in a wild type subject.
15. The method of embodiment 2, wherein the cells comprise a myocyte, and wherein the functional dystrophin protein or a functional fragment thereof is expressed in the myocyte in an amount exceeding 0.5% of a dystrophin level in a wild type subject.
16. The method of embodiment 1, wherein the cell is a myocyte, and wherein the dystrophin protein or a functional fragment thereof is expressed (i) about the cellular membrane of the myocyte, (ii) throughout the cytoplasm of the myocyte, or (iii) both about the cellular membrane and throughout the cytoplasm of the myocyte.
17. The method of embodiment 2, wherein the cells comprise a myocyte, and wherein the functional dystrophin protein or a functional fragment thereof is expressed (i) about the cellular membrane of the myocyte, (ii) throughout the cytoplasm of the myocyte, or (iii) both about the cellular membrane and throughout the cytoplasm of the myocyte.
18. The method of any one of embodiments 4 or 5, wherein the functional dystrophin protein or the functional fragment thereof is expressed in a cell of the subject, wherein the cell is a myocyte.
19. The method of embodiment 7, wherein the functional dystrophin protein or a functional fragment thereof is expressed (i) about the cellular membrane of the myocyte, (ii) throughout the cytoplasm of the myocyte, or (iii) both about the about the cellular membrane and throughout the cytoplasm of the myocyte.
20. The method of any one of embodiments 1 or 3, wherein the method induces uptake of the nucleic acid into at least 0.25% of a population of cells comprising the cell.
21. The method of any one of embodiments 2 or 17, wherein the population of cells is in a cardiac tissue or a heart of the subject, a skeletal muscle tissue or a skeletal muscle of the subject, or a diaphragm tissue or a diaphragm of the subject.
22. The method of embodiment 7, wherein the functional dystrophin protein is expressed in the myocyte in the muscle tissue.
23. The method of embodiment 22, wherein the functional dystrophin protein is expressed along the length of the muscle fiber in the muscle tissue.
24. The method of embodiment 22, wherein the functional dystrophin protein is positioned about the sarcolemma of the muscle fiber in the muscle tissue.
25. The method of any one of embodiments 24, wherein the functional dystrophin protein positioned about the sarcolemma is located along a surface of the sarcolemma along a length of the sarcolemma in a direction parallel to the muscle fiber encapsulated within the sarcolemma.
26. The method of any one of embodiments 22-24, wherein the functional dystrophin protein transfers tension along the length of the muscle fiber or improves a maximum tension loading capacity of the muscle tissue.
27. The method of any one of embodiments 7 or 22-26, wherein at least 1, 3, 5, 10, 15, 20, 25, or 28% of muscle fibers in the treated muscle tissue comprise the nucleic acid encoding the functional dystrophin protein.
28. The method of embodiment 8, wherein at least 6, 7, 8, 9. 10, 11, 12, 13, 14, 15, 16, 17, 18, 19, 20, 21, 22, 23, 24, 25, 26, 27, 28, 29, 30, 35, 40, 45, 50, 55, 60, or 65% of muscle fibers in the muscle tissue of the subject contain the nucleic acid encoding the functional dystrophin protein.
29. The method of embodiment 8 or 28, wherein at least 1% of muscle fibers in the muscle tissue of the subject express the full length dystrophin protein.
30. The method of any one of embodiments 2-8, wherein the cell is a multinucleated cell, a myocyte, or a muscle cell.
31. The method of embodiment 30, wherein the myocyte or the muscle cell comprises a skeletal muscle myocyte, a cardiomyocyte, or a smooth muscle cell.
32. The method of embodiment 31, wherein the myocyte is a skeletal muscle myocyte, and wherein the functional dystrophin protein or fragment thereof is expressed in the myocyte at a level of at least 1, 2, 3, 4, 5, 10, 20, 30, 40, 50, 60, 70, 80, 90, 100, 110, or 120% of normal dystrophin level in a subject not having a dystrophinopathy disorder.
33. The method of embodiment 28, wherein the myocyte or the muscle cell is located in a cardiac tissue or a heart of the subject.
34. The method of embodiment 33, wherein the functional dystrophin protein or fragment thereof is expressed in the cardiac tissue or the heart in an amount of at least 0.3, 0.5, 0.75, 1, 2, 3, 4, 5, 6, 7, 8, 9, or 10% of a dystrophin level in human heart whole tissue lysate as measured by western blot assay.
35. The method of embodiment 30, wherein the myocyte or the muscle cell is located in a skeletal muscle tissue or a skeletal muscle of the subject.
36. The method of embodiment 35, wherein the functional dystrophin protein or fragment thereof is expressed in the muscle tissue or the skeletal muscle in an amount of at least 0.3, 0.5, 0.75, 1, 2, 3, 4, 5% of a dystrophin level in human skeletal muscle whole tissue lysate when measured by western blot assay.
37. The method of embodiment 30, wherein the myocyte or the muscle cell is located in a diaphragm tissue or a diaphragm of the subject.
38. The method of embodiment 37, wherein the functional dystrophin protein or fragment thereof is expressed in the diaphragm tissue or the diaphragm in an amount of at least 0.3, 0.5, 0.75, 1, 2, 3, 4, 5, 6, 7, 8, 9, or 10% of a dystrophin level in human diaphragm whole tissue lysate when measured by western blot assay.
39. The method of any one of embodiments 33, 35, or 37, wherein the functional dystrophin protein or fragment thereof is expressed in the cardiac tissue, the muscle tissue, or the diaphragm tissue, throughout a treated organ comprising the cardiac tissue, throughout a treated organ comprising the muscle tissue, or throughout a treated organ comprising the diaphragm tissue.
40. The method of embodiment 39, wherein the functional dystrophin protein is expressed at detectable levels throughout the treated organ in two or more samples taken from opposite ends of the treated organ.
41. The method of any one of the preceding claims, wherein the nucleic acid or the expression vector is an unencapsulated nucleic acid.
42. The method of any one of embodiments 1, 4, 5, or 7, wherein a coding sequence of the nucleic acid encoding the functional dystrophin protein is at least 5000, 6000, 7000, 8000, 9000, 10000, or 11000 nucleotides.
43. The method of any one of embodiments 2-5, 7, or 8, further comprising administering a sonoactive agent to the subject, and applying ultrasound energy a cell of the subject.
44. The method of any one of embodiments 2-8, wherein a length the functional dystrophin protein is at least 1750, 2000, 2250, 2500, 2750, 3000, or 3250 amino acids.
45. The use of embodiment 6, wherein a coding sequence of the functional dystrophin protein is at least 5000 nucleotides.
46. The use of embodiment 6, wherein the nucleic acid encoding the functional dystrophin protein and the sonoactive agent are configured to induce expression of the functional dystrophin protein in a cell of the subject upon administration of ultrasound energy to the cell.
47. The method of any one of the preceding claims, wherein a coding sequence of the dystrophin protein or the functional dystrophin protein is at least 6000, 7000, 8000, 9000, 10000, or 11000 nucleotides.
48. The method of embodiment 1, wherein a coding sequence of the nucleic acid encoding the dystrophin protein encodes a functional dystrophin protein.
49. The method of embodiment 48, wherein the dystrophin protein is at least 1750, 2000, 2250, 2500, 2750, 3000, or 3250 amino acids in length.
50. The method of any one of the preceding claims, wherein the functional dystrophin protein comprises a full length dystrophin protein.
51. The method of any one of the preceding claims, wherein the functional dystrophin protein is at least 1750, 2000, 2250, 2500, 2750, 3000, or 3250 amino acids in length.
52. The method of any one of the preceding claims, wherein a coding sequence of the nucleic acid encoding the functional dystrophin protein is contiguous.
53. The method of any one of the preceding claims, wherein the nucleic acid encoding (i) the dystrophin protein or (ii) the functional dystrophin protein further comprises a promoter sequence.
54. The method of embodiment 53, wherein the promoter sequence comprises a sequence of any one of SEQ ID NO: 4-5, or 18.
55. The method of any one of the preceding claims, wherein the nucleic acid encoding the (i) dystrophin protein or (ii) the functional dystrophin protein further comprises a post transcriptional regulatory element.
56. The method of embodiment 55, wherein the post transcriptional regulatory element comprises the sequence of any one of SEQ ID NO: 8-10.
57. The method of any one of the preceding claims, wherein the nucleic acid encoding (i) the dystrophin protein or (ii) the functional dystrophin protein further comprises a nuclear targeting sequence.
58. The method of embodiment 57, wherein the nuclear targeting sequence comprises a sequence of SEQ ID NO: 11.
59. The method of any one of the preceding claims, wherein the (i) nucleic acid encoding the dystrophin protein or (ii) the functional dystrophin protein further comprises a post transcriptional regulatory element and a nuclear targeting sequence.
60. The method of embodiment 59, wherein the post transcriptional regulatory element and the nuclear targeting sequence comprise a sequence of SEQ ID NO: 12.
61. The method of any one of the preceding claims, wherein the nucleic acid encoding (i) the dystrophin protein or (ii) the functional dystrophin protein comprises a sequence of any one of SEQ ID NO: 2-3.
62. The method of any one of the preceding claims, wherein the nucleic acid encoding (i) the dystrophin protein or (ii) the functional dystrophin protein comprises a sequence of any one of SEQ ID NO: 6-7.
63. The method of any one of embodiments 30-31, wherein the muscle cell is located within a muscle tissue of the subject, and wherein the method further comprises one or more of: applying heat to the muscle tissue or increasing a blood flow rate in the muscle tissue.
64. The method of embodiment 63, wherein the heat is applied to the muscle tissue using a transcutaneous compress at a temperature of at least 37° C.
65. The method of embodiment 63, wherein the heat is applied to the muscle tissue using ultrasound energy.
66. The method of embodiment 65, the muscle tissue is located in a heart or a diaphragm of the subject.
67. The method of any one of the preceding claims, wherein the cell or the myocyte is in a muscle tissue of the subject, wherein the method further comprises administering a sonoactive agent to the subject, and wherein the administering the sonoactive agent to the subject comprises distributing the sonoactive agent throughout the capillaries of the muscle tissue.
68. The method of any one of the preceding claims, wherein the cell or the myocyte is in a muscle tissue of the subject, wherein the method further comprises applying ultrasound energy to the subject, and wherein the applying the ultrasound energy comprises applying an acoustic radiation force to the muscle tissue.
69. The method of embodiment 68, wherein the applying the acoustic radiation force comprises displacing the muscle tissue.
70. The method of embodiment 69, wherein the muscle tissue is displaced by at least 0.1, 0.2, 0.3, 0.4, 0.5, 0.6, 0.7, 0.8, 0.9, or 1 mm.
71. The method of embodiment 68-69, wherein the applying the acoustic radiation force comprises displacing a volume of blood in capillaries of the muscle tissue.
72. The method of embodiment 71, wherein the method further comprises administering a sonoactive agent to the subject, wherein the displacing the volume of blood in the capillaries of the muscle tissue transmits a pressure wave throughout a vascular system of the muscle tissue, thereby disrupting the sonoactive agent.
73. The method of embodiment 72, wherein the pressure wave does and/or the ultrasound energy applying the acoustic radiation force does not induce vibrational cavitation of the sonoactive agent.
74. The method of any one of embodiments 1 or 41, wherein the applying the ultrasound acoustic energy comprises applying (i) a first ultrasound energy at a MI of up to 0.4 and (ii) a second ultrasound energy at a MI of greater than 0.4 and up to 3.0.
75. The method of any one of embodiments 1 or 41, wherein the applying the ultrasound acoustic energy comprises applying (i) a first ultrasound energy at a MI of up to 0.4 and (ii) a second ultrasound energy at a MI from about 0.8 to about 1.9.
76. The method of any one of embodiments 1 or 41, wherein the applying the ultrasound acoustic energy comprises applying an acoustic radiation force to the subject.
77. The method of embodiment 76, wherein the ultrasound energy is applied at an intensity (ISPTA) of at least 200 mW/cm².
78. The method any one of embodiments 76-77, wherein the ultrasound energy displaces a muscle tissue of the subject by at least 0.1 mm.
79. The method of any one of embodiments 76-78, wherein the ultrasound energy is applied at a pulse length of at least 20 microseconds.
80. The method of any one of embodiments 1 or 41, wherein the sonoactive agent comprises a shell filled with a gas.
81. The method of embodiment 80, wherein the shell comprises a protein stabilized shell or a lipid stabilized shell.
82. The method of embodiment 80, wherein the gas comprises a perfluorinated case.
83. The method of any one of embodiments 1 or 41, wherein the sonoactive agent comprises microbubbles.
84. The method of any one of embodiments 1 or 41, wherein the sonoactive agent comprises nanobubbles.
85. The method of any one of the preceding claims, wherein the subject has a mutation in a gene encoding a dystrophin protein in a genome of the subject.
86. The method of any one of the preceding claims, wherein the subject has a dystrophinopathy disorder.
87. The method of embodiment 86, wherein the dystrophinopathy disorder comprises Duchenne muscular dystrophy (DMD), Becker muscular dystrophy (BMD), or DMD-associated dilated cardiomyopathy.
88. The method of embodiment 87, wherein the dystrophinopathy disorder comprises Duchenne muscular dystrophy (DMD), and wherein the cell is a skeletal muscle myocyte.
89. The method of embodiment 87, wherein the dystrophinopathy disorder comprises Becker muscular dystrophy (BMD), and wherein the cell is a skeletal muscle myocyte.
90. The method of embodiment 87, wherein the dystrophinopathy disorder comprises DMD-associated dilated cardiomyopathy, and wherein the cell is a cardiac myocyte.
91. The method of embodiment 86, wherein administering the nucleic acid, administering the sonoactive agent, and applying the ultrasound energy induces expression of the dystrophin protein or the functional dystrophin protein, thereby treating the subject having the dystrophinopathy disorder.
92. The method of any one of the preceding claims, wherein the method reduces or ameliorates a symptom of the dystrophinopathy disorder in the subject.
93. The method of any one of the preceding claims, wherein the nucleic acid encoding the dystrophin protein or the functional dystrophin protein is not comprised within a viral vector.
94. The method of any one of the preceding claims, further comprising administering a second dose of the nucleic acid encoding the dystrophin protein or the functional dystrophin protein, the sonoactive agent, and the ultrasound energy to the subject.
95. The method of embodiment 94, further comprising administering a third dose of the nucleic acid encoding the dystrophin protein or the functional dystrophin protein, the sonoactive agent, and the ultrasound energy to the subject, wherein the first through third dose of the nucleic acid encoding the dystrophin protein or the functional dystrophin protein, the sonoactive agent, and the ultrasound energy constitutes a triplicate treatment session.
96. The method of embodiment 95, further comprising administering a second triplicate treatment session to the subject.
97. The method of embodiment 96, further comprising administering a third triplicate treatment session to the subject.
98. The method of any one of the preceding claims, wherein the subject is non-embryonic.
99. The method of any one of the preceding claims, wherein the subject is a mammalian subject.
100. The method of any one of the preceding claims, wherein the subject is a primate subject.
101. The method of any one of the preceding claims, wherein the subject is a human subject.
102. The method of any one of the preceding claims, wherein the subject weighs at least 5 kg.
103. A nucleic acid comprising a nucleic acid sequence having at least 80% sequence identity to any one of SEQ ID NO: 2-3.
104. The nucleic acid of embodiment 103, wherein the nucleic acid sequence has at least 81, 82, 83, 84, 85, 86, 87, 88, 89, 90, 91, 92, 93, 94, 95, 96, 97, 98, or 99% sequence identity to any one of SEQ ID NO: 2-3.
105. The nucleic acid of embodiment 103, wherein the nucleic acid sequence is any one of SEQ ID NO: 2-3.
106. The nucleic acid of any one of embodiments 103-105, wherein the nucleic acid sequence comprises at least 80% sequence identity to any one of SEQ ID NO: 16-17.
107. The nucleic acid of any one of embodiments 103-106, wherein the nucleic acid sequence comprises at least 80% sequence identity to any one of SEQ ID NO: 34-36.
108. The nucleic acid of any one of embodiments 103-107, wherein the nucleic acid sequence encodes a dystrophin protein.
109. The nucleic acid of any one of embodiments 103-107, wherein the nucleic acid sequence encodes a full length dystrophin protein.
110. The nucleic acid of any one of embodiments 103-107, wherein the nucleic acid sequence encodes a dystrophin protein at least 3500 amino acids in length.
111. A nucleic acid delivery vector comprising the nucleic acid of any one of embodiments 103-110.
112. Use of the nucleic acid of any one of embodiments 103-111 in a method of treating a subject having a dystrophinopathy disorder requiring a dystrophin gene therapy or a dystrophin protein replacement therapy.
113. A pharmaceutical composition comprising the nucleic acid of any one of embodiments 103-111.
114. Use of the pharmaceutical composition of embodiment 113 in a method of treating a subject having a dystrophinopathy disorder requiring a dystrophin gene therapy or a dystrophin protein replacement therapy.
115. A cell comprising the nucleic acid of any one of embodiments 103-111.
116. A nucleic acid comprising a nucleic acid sequence having at least 80% sequence identity to any one of SEQ ID NO: 4-5.
117. The nucleic acid of embodiment 116, wherein the nucleic acid sequence has at least 81, 82, 83, 84, 85, 86, 87, 88, 89, 90, 91, 92, 93, 94, 95, 96, 97, 98, or 99% sequence identity to any one of SEQ ID NOS: 4-5.
118. The nucleic acid of embodiment 116, wherein the nucleic acid sequence is a muscle tissue specific promoter sequence.
119. The nucleic acid of embodiment 116, wherein the nucleic acid sequence enhances expression of a therapeutic transgene in a muscle cell.
120. The nucleic acid of embodiment 119, wherein the muscle cell comprises a myocyte, a cardiomyocyte, or a smooth muscle cell.
121. The nucleic acid of embodiment 119, wherein the nucleic acid sequence facilitates expression of a transgene coupled to the promoter for at least 30, 60, 90, 120, 150, 180, 210, 240, 270, or 275 days.
122. The nucleic acid of embodiment 119, wherein the nucleic acid sequence is comprised within a nucleic acid delivery vector and coupled to a nucleic acid coding sequence encoding a protein having at least 90% sequence identity to SEQ ID NO: 1.
123. The nucleic acid of embodiment 119, wherein the nucleic acid sequence is comprised within a nucleic acid delivery vector having a sequence of SEQ ID NO: 7.
124. A nucleic acid delivery vector comprising the nucleic acid of any one of embodiments 116-123.
125. Use of the nucleic acid of any one of embodiments 116-124 in a method of treating a subject having a dystrophinopathy disorder requiring a dystrophin gene therapy or a dystrophin protein replacement therapy, comprising administering the nucleic acid to the subject.
126. A pharmaceutical composition comprising the nucleic acid of any one of embodiments 116-124.
127. Use of the pharmaceutical composition of embodiment 126 in a method of treating a subject having a dystrophinopathy disorder requiring a dystrophin gene therapy or a dystrophin protein replacement therapy.
128. A cell comprising the nucleic acid of any one of embodiments 116-124.
129. A kit comprising: 1) a sonoactive agent; and 2) means for expressing a functional dystrophin protein in a subject.
130. A kit comprising: 1) a sonoactive agent; and 2) a nucleic acid of any one of SEQ ID NO: 1-50.
131. A kit comprising: 1) a sonoactive agent; and 2) the nucleic acid of any one of embodiments 103-111, or 116-121.
132. The kit of embodiment 129, further comprising instructions for administering ultrasound acoustic energy to a subject to facilitate expression of the functional dystrophin protein in a subject.
133. The kit of embodiment 131, further comprising instructions for administering ultrasound acoustic energy to a subject to facilitate expression of the nucleic acid in a subject.
134. The kit of any one of embodiments 129-133, wherein the sonoactive agent comprises lipid-stabilized microstructures.
135. The kit of embodiment 134, wherein the lipid-stabilized microstructures comprise a lipid stabilized shell surrounding a perfluorinated gas core.
136. The kit of embodiment 130, wherein the lipid stabilized shell comprises a monomolecular membrane of hydrogenated egg yolk phosphatidyl serine, wherein the perfluorinated gas core comprises perfluorobutane gas.
137. The kit of embodiment 134, wherein the sonoactive agent is a Sonazoid microbubble. 138. The kit of any one of embodiments 129-133, wherein the sonoactive agent comprises protein stabilized microstructures.
139. The kit of embodiment 138, wherein the protein stabilized microstructures comprise an albumin stabilized shell surrounding a perfluorinated gas core.
140. The kit of embodiment 138, wherein the protein stabilized microstructures are Optison microbubbles.
141. The use of embodiments 114 or 127, wherein the dystrophinopathy disorder comprises Duchenne muscular dystrophy (DMD), Becker muscular dystrophy (BMD), or DMD-associated dilated cardiomyopathy.
142. The use of embodiment 141, wherein the dystrophinopathy disorder comprises Duchenne muscular dystrophy (DMD).
143. The use of embodiment 141, wherein the dystrophinopathy disorder comprises Becker muscular dystrophy (BMD).
144. The use of embodiment 141, wherein the dystrophinopathy disorder comprises DMD-associated dilated cardiomyopathy.
145. A method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not conjugated to an antibody.
146. A method of treating a dystrophinopathy disorder in a subject in need thereof comprising administering to the subject a nucleic acid encoding a functional dystrophin protein, expressing the functional dystrophin protein or a functional fragment thereof in a myocyte of the subject, thereby treating the dystrophinopathy disorder, wherein the nucleic acid encoding the functional dystrophin protein is not associated with cationic peptide moieties.
147. Aspects disclosed herein provide a nucleic acid having a sequence of any one of SEQ ID NO: 1-50.
148. A nucleic acid comprising a promoter sequence having at least 90% sequence identity to any one of SEQ ID NOS: 37-40.
149. The nucleic acid of embodiment 143, wherein the promoter sequence comprises at least 91, 92, 93, 94, 95, 96, 97, 98, or 99% sequence identity to any one of SEQ ID NOS: 37-40.
150. The nucleic acid of embodiment 143, wherein the promoter sequence comprises any one of SEQ ID NOS: 37-40.
151. The nucleic acid of embodiment 143, wherein the promoter sequence is configured to increase expression of a nucleic acid encoding a therapeutic protein operably coupled to the promoter sequence.
152. The nucleic acid of embodiment 143, wherein the promoter sequence is configured to increase expression of a nucleic acid encoding a therapeutic protein operably coupled to the promoter sequence in multiple cell types.
153. The nucleic acid of embodiment 151, wherein the promoter sequence is configured to increase expression of the nucleic acid encoding the therapeutic protein in a muscle cell.
154. The nucleic acid of embodiment 151, wherein the promoter sequence is configured to increase expression of the nucleic acid encoding the therapeutic protein in a kidney cell.
155. The nucleic acid of embodiment 151, wherein the promoter sequence is configured to increase expression of the nucleic acid encoding the therapeutic protein in a liver cell.
156. The nucleic acid of embodiment 150, wherein the liver cell is a hepatocyte, or a LSEC.
157. The nucleic acid of embodiment 151, wherein the promoter sequence is configured to increase expression of the nucleic acid encoding the therapeutic protein in a myocyte.
158. A nucleic acid delivery vector comprising the nucleic acid of any one of embodiments 143-156.
159. A pharmaceutical composition comprising the nucleic acid of any one of embodiments 143-156.
160. A cell comprising the nucleic acid of any one of embodiments 143-156.
161. A method of treating a subject comprising administering the nucleic acid of any one of embodiments 143-156 to a subject.
162. The method of embodiment 161, further comprising administering ultrasound to the subject.
163. The method of embodiment 162, further comprising administering a sonoactive agent to the subject.
164. The method of embodiment 161, further comprising administering a lipid nanoparticle or a polymer nanoparticle to the subject.
165. The method of embodiment 161, wherein the nucleic acid is contained within a viral vector.
166. A kit comprising a sonoactive agent and a nucleic acid of any one of SEQ ID NO: 37-40.
167. Use of a nucleic acid encoding a functional dystrophin protein in a method of treating a dystrophinopathy disorder in a subject in need thereof the method comprising delivering a nucleic acid encoding a functional dystrophin protein in a muscle cell in a muscle tissue of a subject, comprising: administering the nucleic acid encoding the dystrophin protein to the subject wherein a coding sequence of the nucleic acid encoding the dystrophin protein is at least 10000 nucleotides; administering a sonoactive agent to the subject; and applying ultrasound energy having an ultrasound intensity (Ispta) of at least 100 mW/cm², a pulse length of at least 20 μs, and a duty cycle of less than 5% in which the ultrasound energy applies an acoustic radiation force to the muscle tissue and displaces the muscle tissue by at least 0.1 mm and displaces a volume of blood in the capillaries of the muscle tissue and transmits a pressure wave throughout a vascular system of the muscle tissue thereby disrupting the sonoactive agent and delivering the nucleic acid encoding the dystrophin protein to at least 5% of muscle fibers in the muscle tissue.

### III. EXAMPLES

The following examples are included for illustrative purposes only and are not intended to limit the scope of the invention.

### Example 1: In-Vitro Evaluation of Varying Dystrophin Nucleic Acid Sequences in Transfection of C2C12 Cells

The following experiment evaluates expression of nucleic acid delivery vectors comprising codon optimized sequences of human dystrophin.

A plurality of variations of codon optimized sequences of human dystrophin were prepared (SEQ ID NO: 2-3, 19-20) and were compared against one another for expression level of the human dystrophin protein. The codon optimized sequences of human dystrophin were placed in a nucleic acid delivery vector under the influence of either a ubiquitous promoter sequence (SEQ ID NO: 18) or a muscle tissue specific promoter sequence (SEQ ID NO: 4). The double stranded circular DNA vectors were produced in an E. coli based method known within the art, and the sequences of the final vectors evaluated are shown in SEQ ID NOS: 21-28.

Transfection began by preparing the media for C2C12 cells. First, a P3000-DNA master mix was prepared by combining 1000 ng of the DNA plasmids (SEQ ID NOS: 21-28) with the P3000 reagent according to the manufacturer's instructions. Concurrently, a Lipofectamine mix was prepared by diluting Lipofectamine reagent in Opti-MEM medium. Following preparation, equal volumes of the P3000-DNA and Lipofectamine mixes were combined to form the DNA-Lipofectamine-P3000 complexes. This mixture was allowed to incubate at room temperature for 5 minutes to ensure complex formation.

Next, 100 μL of the prepared DNA-Lipofectamine-P3000 complex was added to each well of the 12-well plate according to the final plate layout. The cells were incubated under standard culture conditions, and transfection efficiency was assessed by monitoring reporter expression in positive control wells. The expression of the reporter was visualized using a Revolve microscope, and images were captured for analysis.

Cells were then harvested by collecting the cell pellet for further analysis, including protein expression verification via western blotting.

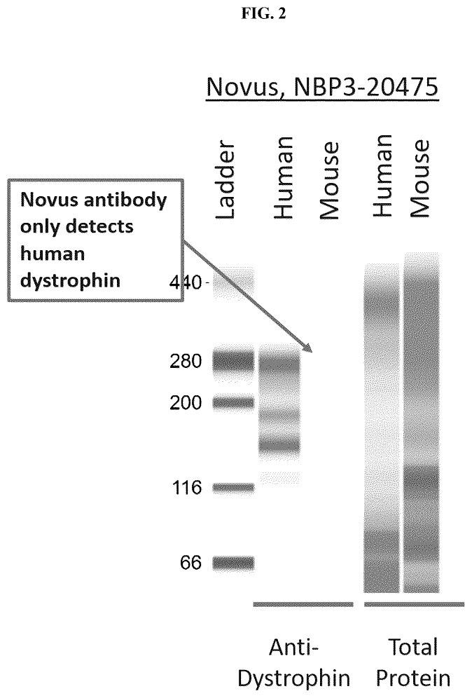

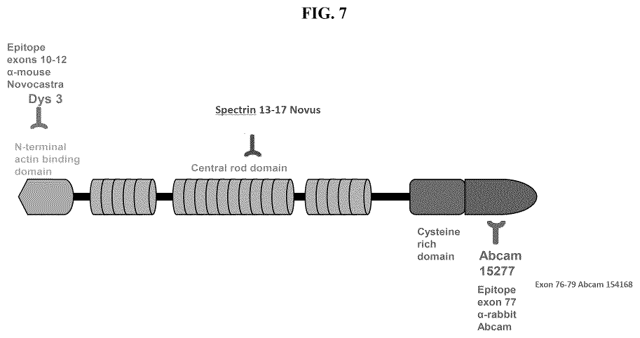

Capillary Western blot evaluation of DMD transfected cells was performed to assess the expression of proteins of interest relative to reference proteins. Capillary Western blot evaluation of DMD ORFs was performed using a Novus Primary antibody, #NBP3-20475 which detects human dystrophin, and does not cross-react with mouse dystrophin. A capillary western blow was performed using Novus Primary antibody, #NBP3-20475 to determine lack of cross reactivity by measuring binding with mouse skeletal lysate and human skeletal muscle tissue lysate, the results of which are show in FIG. 2. Dystrophin is a long, rod-like protein (see FIG. 7) which maintains substantial residual secondary structure due to its elongated spectrin-repeat rod domain, even under boiling, reducing, and SDS conditions, which prevented complete unfolding and thus reduces its apparent molecular weight Western or capillary-based immunoassays because dystrophin migrated anomalously on the SDS-PAGE and Simple Western (Wes) ladders due to dystrophin's rod-like architecture and high proline/coil-content which limited SDS binding, altered its charge-to-mass ratio, and produced migration in the 280-300 kDa range despite the protein's true size. See Beekman et al., Use of capillary Western immunoassay (Wes) for quantification of dystrophin levels in skeletal muscle of healthy controls and individuals with Becker and Duchenne muscular dystrophy, PLOS|ONE (2018) at S1 FIG (“The ladders do not completely line up in the high molecular weight region. The 440 band of Wes ladder migrated slower than 460 band of HiMark ladder, which can lead to underestimation of high molecular weights >280 kDa on the Wes.”); see also id. at FIG. 2D (“The specificity of dystrophin detection by Wes was however confirmed by the fact that 4 different antibodies against dystrophin which recognise different dystrophin epitopes, Mandys106, ab154168, Manex59B and Dys1, all resulted in a signal at 300 kDa in a healthy control sample (FIG. 2D).”); see also Goossens et al., DMD antisense oligonucleotide mediated exon skipping efficiency correlates with flanking intron retention time and target position within the exon, 20:1 RNA Biology 693-702 (2023) (detecting dystrophin protein by Wes capillary immunoassay after treatment of DMD patient with exon skipper 48-50 to produce a long-length but still partially truncated dystrophin protein detected at 280 kDa) and Morisaka et al., CRISPR-Cas3 induces broad and unidirectional genome editing in human cells, 10:5302 Nature Communications (2019) (detecting dystrophin protein by Wes capillary immunoassay after treatment of DMD patient with exon skipper 44 to produce a long-length but still partially truncated dystrophin protein detected at 280 kDa).

Three μl of lysate was diluted to 2 ug/ul. The protocol began with confirming that the centrifuge was at room temperature. Reagents from the EZ Standard pack of the Separation Module were prepared by placing the 5× Mastermix, Ladder, and dithiothreitol (DTT) on ice. To prepare a 400 mM DTT solution, 40 μl of double-distilled water (ddH2O) was added to the DTT tube, the foil seal was pierced with a pipette tip, and the contents were mixed thoroughly. A 10× Sample Buffer was prepared by adding 20 μl to the 5× Mastermix tube along with 20 μl of the DTT solution, and the mixture was pipetted to ensure homogeneity. Additionally, 20 μl of ddH2O was added to the Ladder tube, mixed and stored on ice.

Samples were diluted to the desired concentrations in 0.1% Sample Buffer, with protein lysates from co-immunoprecipitation (Co-IP) experiments diluted at ratios of 50× and 100× using specified volumes. Master mix was added to the diluted samples at a ratio of 1:4 in PCR strip tubes, which were thoroughly mixed and briefly centrifuged. Samples were denatured by incubating at 95° C. for 5 minutes in a thermal cycler, after which they were spun down and placed on ice to minimize evaporation. Reconstitution agents were prepared by sequentially adding 150 μl of Reconstitution Agent 1 and 150 μl of Reconstitution Agent 2 to the designated tube, with gentle pipetting for mixing. Reconstitution Reagents 1 and 2 were combined in a conical tube at a ratio of 1.4 mL to 0.35 mL and vortexed briefly. A detection reagent mixture of 450 μl Luminol-S and 450 μl peroxide was prepared and kept on ice.

The primary antibody was diluted at either 1:50 or 1:100 in Antibody Diluent 2, depending on the requirements. Reagents were dispensed into a plate as per predefined maps, with the foil cover's top portion removed for access. The plate was covered with a lid, balanced carefully in the centrifuge, and centrifuged at 2500 rpm for 5 minutes at room temperature, and. Using the ProteinSimple software, a new assay was created with parameters suitable for the specific experimental setup. The instrument door of a Jess Automated Western Blot System (Jess) door was opened by activating the top silver button, and the capillary cartridge was inserted into the holder, with the internal light shifting from orange to blue.

Following centrifugation, the plate lid was removed, and the evaporative seal on the foil's bottom portion was peeled off. Bubbles in the Separation matrix wells were eliminated using a pipette tip before the plate was placed in the holder within the Jess instrument. The door was closed, and the assay was initiated via the software's Start button, with continuous monitoring to prevent errors. Upon completion of the assay, the plate was disposed of in biosafety level (BSL) waste, and the cartridge was discarded in sharps waste.

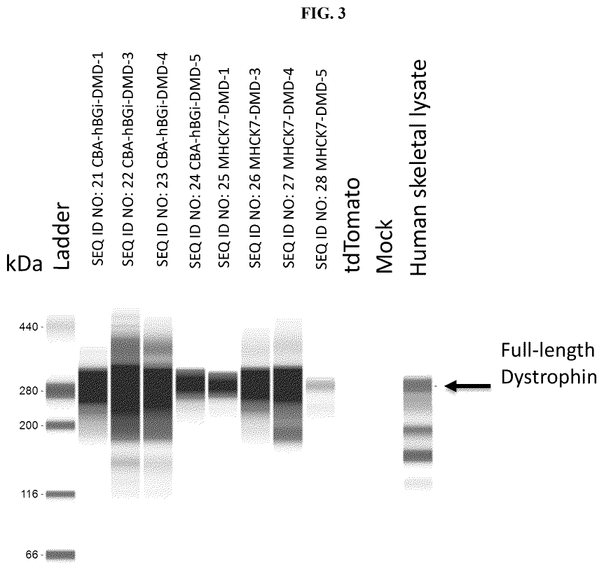

Band results of the capillary western blot are shown in FIG. 3, in which it is observed that some codon optimized sequences of human dystrophin SEQ ID NO: 2-3 were highly expressed under the influence of both the ubiquitous promoter sequence (hBGi; SEQ ID NO: 18) or a muscle tissue specific promoter sequence (MHCK7; SEQ ID NO: 4), while some codon optimized sequences of human dystrophin SEQ ID NO: 19-20 exhibited only modest expression under the influence of both the ubiquitous promoter sequence (SEQ ID NO: 18) or a muscle tissue specific-promoter sequence (SEQ ID NO: 4).

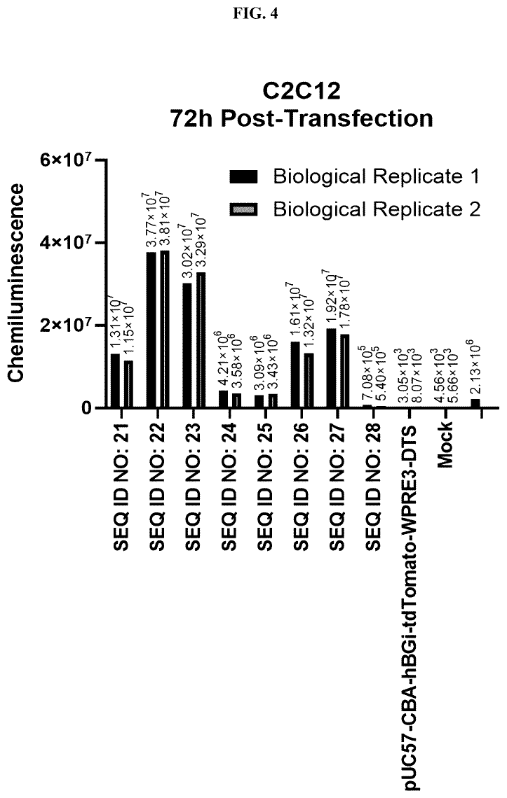

Chemiluminescence was utilized to produce a quantitative readout of expression levels using a secondary antibody configured to bind Novus Primary antibody, #NBP3-20475. Results are shown in FIG. 4 in which it is observed that codon optimized sequences of human dystrophin SEQ ID NO: 2-3 under the influence of a ubiquitous promoter sequence (SEQ ID NO: 18) contained within vectors SEQ ID NO: 22 and 33, respectively, were highly expressed, exhibiting chemiluminescence values of about 3.8 E7 and about 3.2 E7, respectively; and codon optimized sequences of human dystrophin SEQ ID NO: 2-3 under the influence of a muscle specific promoter SEQ ID NO: 4 contained within vectors SEQ ID NO: 26 and 27, respectively, were highly expressed exhibiting chemiluminescence values of about 1.4 E7 and about 1.8 E7 respectively. In both cases, the codon optimized sequences of human dystrophin SEQ ID NO: 2-3 exhibited 3-10 fold greater expression then other codon optimized sequences of human dystrophin SEQ ID NO: 19 contained within vector sequences SEQ ID NO: 21 and 25, and codon optimized sequences of human dystrophin SEQ ID NO: 20 contained within vector sequences SEQ ID NO 24 and 28.

### Example 2: In-Vivo Evaluation of Muscle Tissue Specific Vector and Expression of Full-Length Dystrophin Protein in a Murine Model of Sonoporation to the Skeletal Muscle

In this example, nucleic acid delivery vectors with expression cassettes comprising promoter sequences which are optimized for expression in skeletal myocytes are evaluated in a murine model of ultrasound mediated nucleic acid delivery to the skeletal muscle (quadriceps).

### Experimental Animals and Protocol

Five groups of RAG2 mice were evaluated. Prior to the experiment, mice were implanted with a jugular vein catheter though which the sonoactive agent and the DNA solution was administered. The experimental groups were set forth in Table 1 below. In brief, Group 1 was a no-ultrasound control administered only the vector and the sonoactive agent, Groups 2-3 were administered vectors comprising a gene encoding a fluorescent reporter protein (FLUC) under either a ubiquitous or a muscle tissue specific promoter with administration of a sonoactive agent and ultrasound, and Groups 4-5 were administered vectors comprising codon optimized sequences of human dystrophin, SEQ ID NO: 3, under either a ubiquitous or a muscle tissue specific promoter with administration of a sonoactive agent and ultrasound. Optison (human serum albumin stabilized perflutran core microbubbles) was used as the ultrasound contrast agent in this experiment.

TABLE 1
In-Vivo Evaluation of Muscle Tissue Specific Vector and Expression of Full-Length
Dystrophin Protein in a Murine Model of Sonoporation to the Skeletal Muscle

| | | DNA | | | | |
|---|---|---|---|---|---|---|
| Group | | Dose | Sonoactive | # of | | |
| (n = 3) | Strain | (ug) | agent | doses | Ultrasound | Vector |
| 1 | Rag2 | 250 | Optison | 3 | None | SEQ ID NO: 29 (ubiquitous promoter + |
| | | | | | | FLUC reporter) |
| 2 | Rag2 | 250 | Optison | 3 | L8-18, MI 1.4, 10 | SEQ ID NO: 29 (ubiquitous promoter + |
| | | | | | sec on, 20 sec off, | FLUC reporter) |
| | | | | | 60 sec total on | |
| 3 | Rag2 | 250 | Optison | 3 | L8-18, MI 1.4, 10 | SEQ ID NO: 30 (muscle tissue specific |
| | | | | | sec on, 20 sec off, | promoter + FLUC reporter) |
| | | | | | 60 sec total on | |
| 4 | Rag2 | 250 | Optison | 3 | L8-18, MI 1.4, 10 | SEQ ID NO: 31 (ubiquitous promoter + |
| | | | | | sec on, 20 sec off, | DMD Codon Optimized SEQ ID NO: 3) |
| | | | | | 60 sec total on | |
| 5 | Rag2 | 250 | Optison | 3 | L8-18, MI 1.4, 10 | SEQ ID NO: 32 (muscle tissue specific |
| | | | | | sec on, 20 sec off, | promoter + DMD Codon Optimized SEQ ID |
| | | | | | 60 sec total on | NO: 3) |

The mice received application of ultrasound energy following administration of 50 μL of the sonoactive agent and one third of the DNA payload in each treatment session. In a treatment session, the left leg of each animal was placed in a heated water bath at 38° C. for 2 minutes prior to applying ultrasound using a L8-18 probe on a General Electric LOGIQ E9 system in Elasto mode, the probe applying an acoustic radiation force at a frequency of about 2.5 MHz at a mechanical index of 1.4 at a 100% push output at an ultrasound intensity of at least 200 mW/cm² (ISPTA) for about 10 sec on, 20 sec off, for a total of 60 sec (2 cycles) of ultrasound application over the left quadriceps region of each mouse. A second identical treatment session was applied at 24 hours after the first treatment session administering the second one third of the DNA payload, and a third identical treatment session was applied at 24 hours after the second treatment session administering the last one third of the DNA payload.

For Groups 1-3, the mice were injected with D-Luciferin, potassium salt and imaged using IVIS: 1 day following the first treatment session; 1 day following the second treatment session; and 1, 2 and 6 days following the third treatment session.

For Groups 4-5, necropsy was performed 4 days following the third treatment session. Tissue samples were collected, placed into a sterile tube, and snap frozen using liquid nitrogen (N₂). Tissue samples were directly transferred to −80° C. storage.

Capillary Western blot evaluation of DMD transfected samples was performed to assess the expression of DMD. Capillary western blot analysis was performed as described in Example 1 but using Abcam antibody for dystrophin (ab154168) which is specific to human dystrophin, with the addition of human skeletal muscle lysate which was normalized to 2 ug/ul, then diluted to create standards at 100, 50, 25, 5, 2, and 1%. A 2 μg/uL was the selected concentration for the western blot analysis. Samples were then diluted to 2 μg/uL and compared with human skeletal muscle lysate standards. Dystrophin is a long, rod-like protein (see FIG. 7) which maintains substantial residual secondary structure due to its elongated spectrin-repeat rod domain, even under boiling, reducing, and SDS conditions, which prevented complete unfolding and thus reduces its apparent molecular weight Western or capillary-based immunoassays because dystrophin migrated anomalously on the SDS-PAGE and Simple Western (Wes) ladders due to dystrophin's rod-like architecture and high proline/coil-content which limited SDS binding, altered its charge-to-mass ratio, and produced migration in the 280-300 kDa range despite the protein's true size. See Beekman et al., Use of capillary Western immunoassay (Wes) for quantification of dystrophin levels in skeletal muscle of healthy controls and individuals with Becker and Duchenne muscular dystrophy, PLOS|ONE (2018) at S1 FIG (“The ladders do not completely line up in the high molecular weight region. The 440 band of Wes ladder migrated slower than 460 band of HiMark ladder, which can lead to underestimation of high molecular weights >280 kDa on the Wes.”); see also id. at FIG. 2D (“The specificity of dystrophin detection by Wes was however confirmed by the fact that 4 different antibodies against dystrophin which recognise different dystrophin epitopes, Mandys106, ab154168, Manex59B and Dys1, all resulted in a signal at 300 kDa in a healthy control sample (FIG. 2D).”); see also Goossens et al., DMD antisense oligonucleotide mediated exon skipping efficiency correlates with flanking intron retention time and target position within the exon, 20:1 RNA Biology 693-702 (2023) (detecting dystrophin protein by Wes capillary immunoassay after treatment of DMD patient with exon skipper 48-50 to produce a long-length but still partially truncated dystrophin protein detected at 280 kDa) and Morisaka et al., CRISPR-Cas3 induces broad and unidirectional genome editing in human cells, 10:5302 Nature Communications (2019) (detecting dystrophin protein by Wes capillary immunoassay after treatment of DMD patient with exon skipper 44 to produce a long-length but still partially truncated dystrophin protein detected at 280 kDa).

### Results

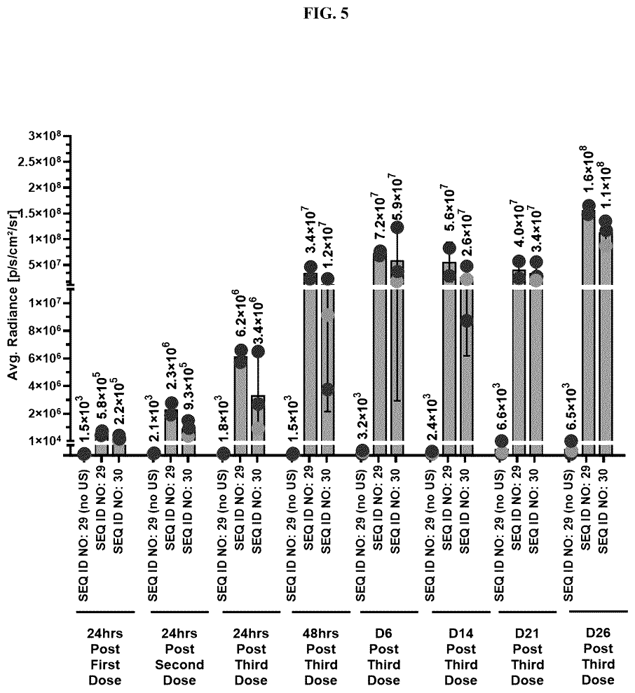

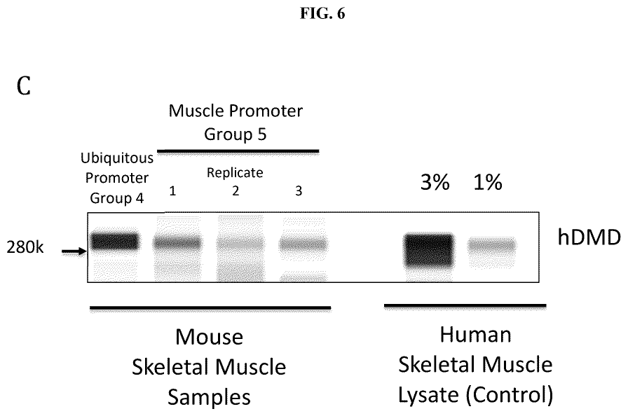

Results for Groups 1-3 are shown in FIG. 5, in which it is shown that the negative control group 1 exhibits only background levels of average radiance at all time points; Group 2 administered the vector of SEQ ID NO: 29 (ubiquitous promoter+FLUC reporter) exhibited average radiance levels of about 2.3 E6 p/s/cm2/sr at 24 hours following the second treatment, average radiance levels of about 6.2 E6 p/s/cm2/sr at 24 hours following the third treatment, average radiance levels of about 3.44 E7 p/s/cm2/sr at 48 hours following the third treatment, and average radiance levels of about 7.2 E7 p/s/cm2/sr at 72 hours following the third treatment; and Group 3 administered the vector of SEQ ID NO: 30 (muscle tissue specific promoter+FLUC reporter) exhibited average radiance levels of about 9.3 E5 p/s/cm2/sr at 24 hours following the second treatment, average radiance levels of about 3.4 E6 p/s/cm2/sr at 24 hours following the third treatment, average radiance levels of about 1.2 E7 p/s/cm2/sr at 48 hours following the third treatment, and average radiance levels of about 5.9 E7 p/s/cm2/sr at 72 hours following the third treatment. The mice were followed for up to 26 days following the third treatment, at which point Group 2 administered the vector of SEQ ID NO: 29 (ubiquitous promoter+FLUC reporter) exhibited average radiance levels of about 1.6 E8 p/s/cm2/sr; and Group 3 administered the vector of SEQ ID NO: 30 (muscle tissue specific promoter+FLUC reporter) exhibited average radiance levels of about 1.1 E8 p/s/cm2/sr. Results of the capillary western blot are shown in FIG. 6, in which samples were diluted to 2 μg/uL and compared with human skeletal muscle lysate standards. In FIG. 6, it is observed that Group 4 (administered the vector of SEQ ID NO: 31 comprising a ubiquitous promoter coupled to DMD Codon Optimized SEQ ID NO: 3) exhibited strong expression of the full length dystrophin protein consistent with the 3% human skeletal muscle lysate standard, while Group 5 (administered the vector of SEQ ID NO: 32 comprising a muscle tissue specific promoter coupled to DMD Codon Optimized SEQ ID NO: 3) exhibited expression of the full length dystrophin protein consistent with the 1% human skeletal muscle lysate standard.

### Example 3: In-Vivo Evaluation of Muscle Tissue Specific Vector and Expression of Full-Length Dystrophin Protein in a Murine Model of Sonoporation to the Diaphragm

In this example, nucleic acid delivery vectors with expression cassettes comprising promoter sequences which were optimized for expression in skeletal myocytes cells were evaluated in a murine model of ultrasound mediated nucleic acid delivery to the diaphragm muscle tissue.

### Experimental Animals and Protocol

Three groups of MDX mice (a mouse model which carries a point mutation in the mouse dystrophin gene (DMD) leading to a lack of functional dystrophin protein) were evaluated. Prior to the experiment, mice were implanted with a jugular vein catheter though which the sonoactive agent and the DNA solution were administered. The experimental groups used are set forth in Table 2 below. In brief, Group 1 was a no-ultrasound control group administered only the vector dose and the sonoactive agent, Group 2 was administered vectors comprising codon optimized sequences of human dystrophin SEQ ID NO: 3 under a muscle tissue specific promoter of SEQ ID NO: 4 (the combination of which being comprised within the expression cassette of SEQ ID NO: 34) with administration of a sonoactive agent and ultrasound and were evaluated for expression effect resulting from repeated dosing increases among the groups, and Group 3 was administered vectors comprising a gene encoding a fluorescent reporter protein under a muscle tissue specific promoter with administration of a sonoactive agent and ultrasound. Sonazoid (phospholipid stabilized perfluorobutane core microbubbles) was used as the sonoactive agent in this experiment.

TABLE 2
In-Vivo Evaluation of Muscle Tissue Specific Vector and Expression of Full-
Length Dystrophin Protein in a Murine Model of Sonoporation to the Diaphragm

| | | DNA | | | | |
|---|---|---|---|---|---|---|
| Group | | Dose | Sonoactive | # of | | Vector/Expression Cassette/Promoter |
| (n = 3) | Strain | (ug) | agent | doses | Ultrasound | Transgene |
| 1 | MDX | 250 | Sonazoid | 3 | None | SEQ ID NO: 27 (expression cassette of SEQ |
| (n = 1) | | | | | | ID NO: 34 with muscle tissue specific |
| | | | | | | promoter of SEQ ID NO: 4 + DMD Codon |
| | | | | | | Optimized SEQ ID NO: 3) |
| 2 | MDX | 250 | Sonazoid | 3 | L8-18, MI 1.4, 2 | SEQ ID NO: 27 (expression cassette of SEQ |
| | | | | | sec on/2 sec off, | ID NO: 34 with muscle tissue specific |
| | | | | | 60 sec total on | promoter of SEQ ID NO: 4 + DMD Codon |
| | | | | | | Optimized SEQ ID NO: 3) |
| 3 | MDX | 250 | Sonazoid | 3 + | L8-18, MI 1.4, 2 | SEQ ID NO: 30 (muscle tissue specific |
| | | | | 3 + | sec on/2 sec off, | promoter of SEQ ID NO: 4 + FLUC reporter) |
| | | | | 3 | 60 sec total on | |

The mice received application of ultrasound energy following administration of 50 μL of the sonoactive agent and one third of the DNA payload in each treatment session. In a treatment session, the middle abdomen region was targeted with ultrasound using a L8-18 probe on a General Electric LOGIQ E9 system in Elasto mode applying an acoustic radiation force at a frequency of about 6.25 MHz at a mechanical index of 1.4 at a 100% push output at an ultrasound intensity of at least 200 mW/cm2 (ISPTA) alternating between 2 seconds on of ultrasound application and 2 seconds off of ultrasound application, for a total of 60 sec of on ultrasound application (30 cycles) over the diaphragm region of each mouse. A second identical treatment session was applied at 3 days following the first treatment session, and a third identical treatment session was applied at 3-4 days after the second treatment session. Each set of three identical treatment sessions is referred to herein as a triplicate treatment session. Group 3 was administered three triplicate treatment sessions.

For Group 3, mice were injected with D-Luciferin, potassium salt and imaged using IVIS weekly following each triplicate treatment session.

For Groups 1-2, 14 days following the first triplicate treatment session, subjects were sacrificed and tissue samples were collected for further analysis.

For Groups 1-2, necropsy was performed at the stated time points, and tissue samples were collected, placed into a sterile tube, and snap frozen using liquid N₂. Tissue samples were directly transferred to −80° C. storage.

Capillary Western blot evaluation of DMD transfected samples was performed to assess the expression of DMD. Capillary western blot analysis was performed as described in Example 1, but using Abcam antibody for dystrophin (ab154168) which is specific to human dystrophin, with the addition of human diaphragm whole tissue lysate (Novus Bio) normalized to 1 mg/mL, then diluted to create standards at 5%, 2%, and 1%. A 1 mg/mL concentration was the selected concentration for the western blot analysis (ab154168 (1:150)). Samples were then diluted to 1 mg/mL and compared with human diaphragm whole tissue lysate standards.

Transgenic tissue samples were stained and imaged using Dystrophin DAB (3,3′-diaminobenzidine) chromogen staining, and hematoxylin nuclei staining. Slides were pre-baked at 60° C. for at least 15 minutes and positioned on a black slide tray. Labels were generated and affixed onto the slides, which were then transferred into the Slide Staining Assemblies (SSA). The Polymer Refine Detection Kit was registered on the Leica controller, and all required reagents were verified. The Refine reagent kit was loaded onto the reagent platform for dip testing. Antibodies specific to the target protein (human dystrophin) were prepared and dispensed into titration tubes. Up to 5 mL of antibody was used for a maximum 30-slide run. The necessary antibodies were loaded onto the reagent platform, and dip testing proceeded. Bulk reagents were replenished as needed, and waste containers were emptied. After dip testing, the SSA buttons were pressed to lower and lock the slide trays. The Leica Bond RX Fully Automated Research Stainer was used for the staining procedure. Once staining concluded, the SSA buttons were pressed again to release the slide trays. Covertiles were removed, and slides were placed in a staining rack. Slides were washed in deionized water for three minutes and allowed to dry at 60° C. for a minimum of 30 minutes. Coverslipping was performed using mounting media, and slides were left to dry overnight for optimal imaging quality. The stained slides were scanned using an Olympus VS200 Slide Scanner to assess dystrophin protein presence in the transgenic tissue samples.

### Results

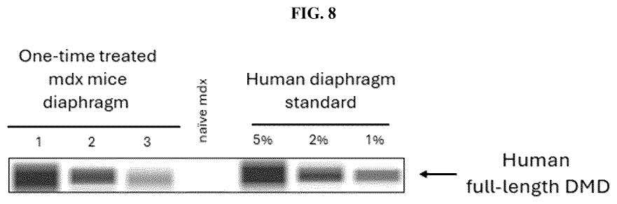

Results of the capillary western blot for Group 2 are shown in FIG. 8 (each lane showing samples collected from a different subject MDX mouse in Group 2), in which samples are diluted to 1 mg/mL and compared with human diaphragm whole tissue lysate (Novus Bio) normalized to 1 mg/mL and diluted to 5, 2, and 1% standards.

Lanes 1, 2 and 3, labeled “one-time treated MDX diaphragm” in FIG. 8, correspond to different subjects from Group 2, and exhibit expression levels of full length human dystrophin, which are comparable to the human diaphragm whole tissue lysate 5, 2, and 1% standards respectively. A sample from a naive MDX mouse diaphragm is also shown as a negative control, and the assay indicates no detectable human dystrophin in the naïve MDX sample, validating the specificity of the antibody and the sensitivity assay for human dystrophin.

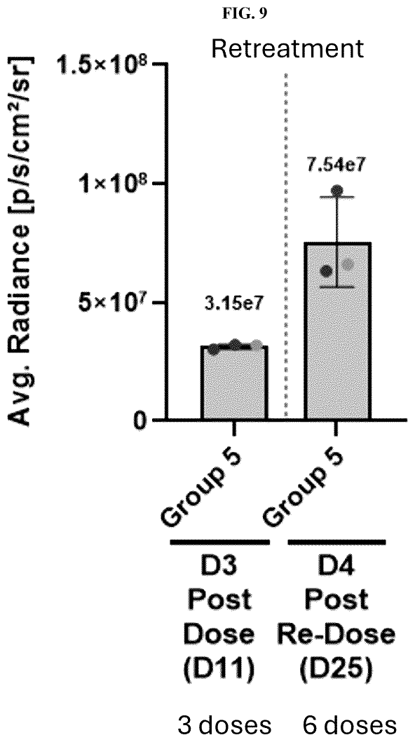

IVIS results for Groups 3 are shown in FIG. 9, in which it is shown that Group 3, administered the vector of SEQ ID NO: 30 (muscle tissue specific promoter+FLUC reporter), exhibited average radiance levels of about 3.15 E7 p/s/cm2/sr at 3 days following the final dose of the first triplicate treatment session (day 11 of the study), and average radiance levels of about 7.54 E7 p/s/cm2/sr at 3 days following the final dose of the second triplicate treatment session (day 25 of the study).

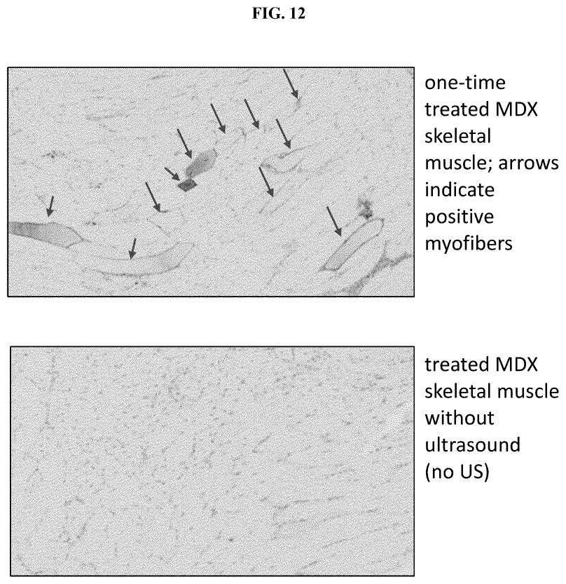

Results of the Dystrophin DAB chromogen staining, and hematoxylin nuclei staining of samples taken from the top performing mouse in Group 2 are shown in FIG. 12 in which expression of the full length dystrophin protein can be observed in transfected myocytes of the subject in both the cellular membrane of the myocyte and throughout the cytoplasm of the myocyte, as shown by the orange and dark orange DAB staining in FIG. 12. Blue dots represent individual nuclei within the tissue samples, as stained with hematoxylin.

### Example 4: In-Vivo Evaluation of Muscle Tissue Specific Vector and Expression of Full-Length Dystrophin Protein in a Murine Model of Sonoporation to the Heart

In this example, nucleic acid delivery vectors with expression cassettes comprising promoter sequences which were optimized for expression in skeletal myocytes were evaluated in a murine model of ultrasound mediated nucleic acid delivery to cardiac tissue.

### Experimental Animals and Protocol

Two groups of MDX mice (a mouse model which carries a point mutation in the mouse dystrophin gene (DMD) leading to a lack of functional dystrophin protein) were evaluated. Prior to the experiment, mice were implanted with a jugular vein catheter though which the sonoactive agent and the DNA solution were administered. The experimental groups used are set forth in Table 3 below. In brief, Group 1 was a no-ultrasound control group administered only the vector dose and the sonoactive agent, Group 2 was administered codon optimized sequences of human dystrophin SEQ ID NO: 3 under a muscle tissue specific promoter of SEQ ID NO: 4 (the combination of which being comprised within the expression cassette of SEQ ID NO: 34) with administration of a sonoactive agent and ultrasound and was evaluated for the expression effect resulting from repeated dosing increases among the groups. Sonazoid (phospholipid stabilized perfluorobutane core microbubbles) was used as the sonoactive agent in this experiment.

TABLE 3
In-Vivo Evaluation of Muscle Tissue Specific Vector and Expression of Full-
Length Dystrophin Protein in a Murine Model of Sonoporation to the Heart

| | | DNA | | | | |
|---|---|---|---|---|---|---|
| Group | | Dose | Sonoactive | # of | | Vector/Expression |
| (n = 5) | Strain | (ug) | agent | doses | Ultrasound | Cassette/Promoter/Transgene |
| 1 | MDX | 80 | Sonazoid | 3 | None | SEQ ID NO: 27 (expression cassette of |
| (n = 1) | | | | | | SEQ ID NO: 34 with muscle tissue specific |
| | | | | | | promoter of SEQ ID NO: 4 + DMD Codon |
| | | | | | | Optimized SEQ ID NO: 3) |
| 2 | MDX | 80 | Sonazoid | 3 | L8-18, MI 0.46, | SEQ ID NO: 27 (expression cassette of |
| | | | | | PO 30%, 40 sec | SEQ ID NO: 34 with muscle tissue specific |
| | | | | | on | promoter of SEQ ID NO: 4 + DMD Codon |
| | | | | | | Optimized SEQ ID NO: 3) |

The mice received application of ultrasound energy following administration of 50 uL of the sonoactive agent and one third of the DNA payload in each treatment session. In a treatment session, the upper abdomen region was targeted with ultrasound using a L8-18 probe on a General Electric LOGIQ E9 system in Elasto mode applying an acoustic radiation force at a frequency of about 6.25 MHz at a mechanical index of 0.46 at a 30% push output at an ultrasound intensity of at least 200 mW/cm2 (ISPTA) applying ultrasound for 40 seconds over the cardiac region of each mouse. A second identical treatment session was applied at 3 days following the first treatment session, and a third identical treatment session was applied at 3-4 days after the second treatment session. Each set of three identical treatment sessions is referred to herein as a triplicate treatment session.

For Groups 1-2, 14 day following the first triplicate treatment session, subjects were sacrificed and tissue samples were collected for further analysis.

For Groups 1-2, necropsy was performed at the stated time points, and tissue samples were collected, placed into a sterile tube, and snap frozen using liquid N₂. Tissue samples were directly transferred to −80° C. storage. Capillary western blot analysis was performed as described in Example 1, with the addition of human heart whole tissue lysate (Novus Bio) normalized to 1 mg/mL, then diluted to create standards at 10, 5 and 2%. A 1 mg/mL concentration was the selected concentration for the western blot analysis (ab154168 (1:150)). Samples were then diluted to 1 mg/mL and compared with human heart whole tissue lysate.

### Results

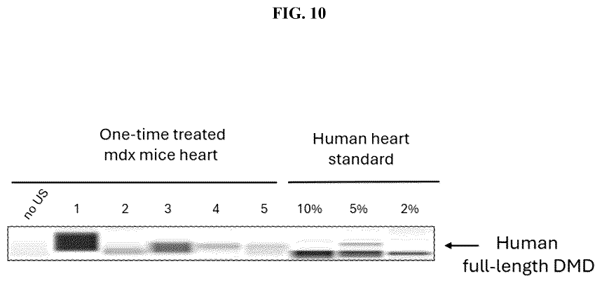

Results of the capillary western blot are shown for Group 2 in FIG. 10 (each lane showing samples collected from a different subject MDX mouse in Group 2), in which samples are diluted to 1 mg/mL and compared with human diaphragm whole tissue lysate (Novus Bio) normalized to 1 mg/mL and diluted to 10, 5 and 2% standards.

Lanes 1-5, labeled “one-time treated MDX heart” in FIG. 10, correspond to different subjects from Group 2, and exhibit expression levels of full length human dystrophin which are comparable to the human diaphragm whole tissue lysate 10, 5 and 2% standard, with the Lane 1 subject exhibiting an expression level most similar to the 10% standard. A sample from Group 1 not administered any ultrasound is also shown as a negative control, and the assay indicates no detectable human dystrophin in this sample.

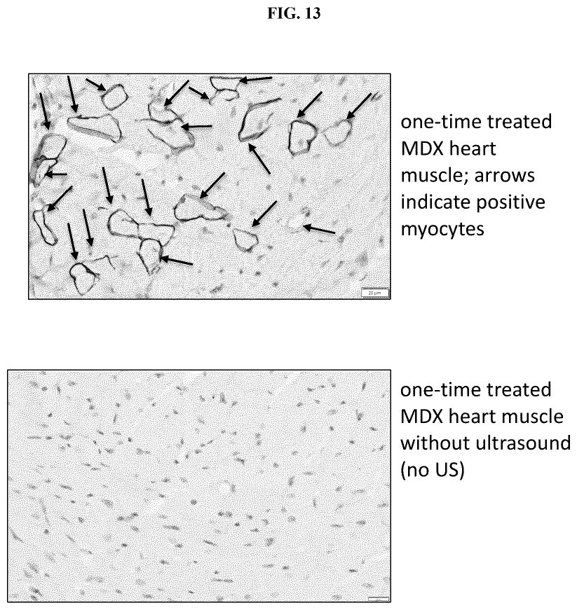

Staining was performed as described in Example 3. Results of the DAB staining are shown in FIG. 13 for the top performing mice in Group 2 in which expression of the full length dystrophin protein can be observed in transfected myocytes of the subject about the cellular membrane of the myocyte and throughout the cytoplasm of the myocyte, as shown by the orange and dark orange DAB staining in FIG. 13. Blue dots represent individual nuclei within the tissue samples stained with hematoxylin.

### Example 5: In-Vivo Evaluation of Muscle Tissue Specific Vector and Expression of Full-Length Dystrophin Protein in a Murine Model of Sonoporation to the Skeletal Muscle

In this example, nucleic acid delivery vectors with expression cassettes comprising promoter sequences which were optimized for expression in skeletal myocytes were evaluated in a murine model of ultrasound mediated nucleic acid delivery to the skeletal muscle (quadriceps).

### Experimental Animals and Protocol

Two groups of MDX mice (a mouse model which carries a point mutation in the mouse dystrophin gene (DMD) leading to a lack of functional dystrophin protein) were evaluated. Prior to the experiment, mice were implanted with a jugular vein catheter though which the sonoactive agent and the DNA solution were administered. The experimental groups used are set forth in Table 4 below. In brief, Group 1 was a no-ultrasound control group administered only the vector dose and the sonoactive agent, Group 2 was administered vectors comprising codon optimized sequences of human dystrophin SEQ ID NO: 3 under a muscle tissue specific promoter of SEQ ID NO: 4 (the combination of which being comprised within the expression cassette of SEQ ID NO: 34) with administration of a sonoactive agent and ultrasound and were evaluated for expression effects resulting from repeated dosing increases among the groups. Optison (human serum albumin stabilized perflutran core microbubbles) was used as the ultrasound contrast agent in this experiment.

TABLE 4
In-Vivo Evaluation of Muscle Tissue Specific Vector and Expression of Full-Length
Dystrophin Protein in a Murine Model of Sonoporation to the Skeletal Muscle

| | | DNA | | | | |
|---|---|---|---|---|---|---|
| Group | | Dose | Sonoactive | # of | | Vector/Expression Cassette/Promoter/ |
| (n = 3) | Strain | (ug) | agent | doses | Ultrasound | Transgene |
| 1 | MDX | 250 | Optison | 3 | None | SEQ ID NO: 27 (expression cassette of |
| (n = 1) | | | | | | SEQ ID NO: 34 with muscle tissue specific |
| | | | | | | promoter of SEQ ID NO: 4 + DMD Codon |
| | | | | | | Optimized SEQ ID NO: 3) |
| 2 | MDX | 250 | Optison | 3 | L8-18, MI 1.4, 2 | SEQ ID NO: 27 (expression cassette of |
| | | | | | sec on/2 sec off, | SEQ ID NO: 34 with muscle tissue specific |
| | | | | | 60 sec total on | promoter of SEQ ID NO: 4 + DMD Codon |
| | | | | | | Optimized SEQ ID NO: 3) |

The mice received application of ultrasound energy following administration of 50 uL of the sonoactive agent and one third of the DNA payload in each treatment session. In a treatment session, the middle thigh region was targeted ultrasound using a L8-18 probe on a General Electric LOGIQ E9 system in Elasto mode applying an acoustic radiation force at a frequency of about 6.25 MHz at a mechanical index of 1.4 at a 100% push output at an ultrasound intensity of at least 200 mW/cm2 (ISPTA) alternating between 2 seconds on of ultrasound application and 2 seconds off of ultrasound application, for a total of 60 sec of on ultrasound application over the quadriceps region of each mouse. A second identical treatment session was applied at 3 days following the first treatment session, and a third identical treatment session was applied at 3-4 days after the second treatment session, thereby applying a triplicate treatment session to the subjects.

For Groups 1-2, 14 day following the triplicate treatment session subjects were sacrificed and tissue samples were collected for further analysis.

Necropsy was performed at the stated time points, and tissue samples were collected, placed into a sterile tube, and snap frozen using liquid N₂. Tissue samples were directly transferred to −80 C storage. Capillary western blot analysis was performed as described in Example 1, with the addition of human heart whole tissue lysate (Novus Bio) normalized to 1 mg/mL, then diluted to create standards at 10, 5 and 2%. A 1 mg/mL concentration was the selected concentration for the western blot analysis (ab154168 (1:150)). Samples were then diluted to 1 mg/mL and compared with human heart whole tissue lysate.

### Results

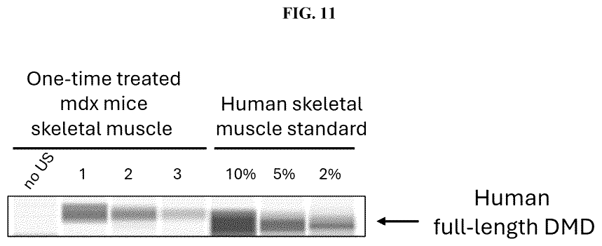

Results of the capillary western blot for Group 2 are shown in FIG. 11 (each showing samples collected from a different subject MDX mouse in Group 2), in which samples are diluted to 1 mg/mL and compared with human diaphragm whole tissue lysate (Novus Bio) normalized to 1 mg/mL and diluted to 10, 5 and 2% standards.

Lanes 1-3 labeled “one-time treated MDX skeletal muscle” in FIG. 11 correspond to different subjects from Group 2, and exhibit expression levels of full length human dystrophin which are comparable to the human diaphragm whole tissue lysate 5 and 2% standard, with the Lane 1 subject exhibiting an expression level most similar to the 5% standard. A sample from Group 1 not administered any ultrasound is also shown as a negative control, and the assay indicates no detectable human dystrophin in this sample.

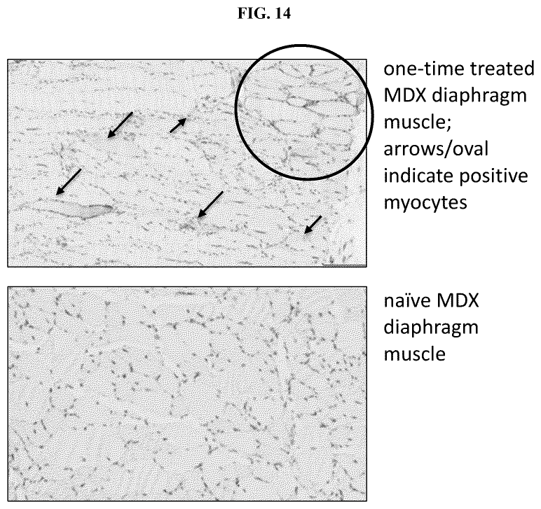

Staining was performed as described in Example 3. Results of the DAB staining is shown in FIG. 14 in which expression of the full length dystrophin protein can be observed in transfected myocytes of the subject both in the cellular membrane of the myocyte and throughout the cytoplasm of the myocyte, as shown by the orange and dark orange DAB staining in FIG. 14 Blue dots represent individual nuclei within the tissue samples stained with hematoxylin.

### Example 6: In-Vivo Evaluation of Muscle Tissue Specific Vector and Expression of Full-Length Dystrophin Protein in a NHP Model of Sonoporation to the Skeletal Muscle

In this example, nucleic acid delivery vectors with expression cassettes comprising promoter sequences which are optimized for expression in skeletal myocytes cells are evaluated in a non-human primate model of ultrasound mediated nucleic acid delivery to the skeletal muscle (quadriceps).

### Experimental Animals and Protocol

The subject was one male cynomolgus macaque (cyno). and was infused with phospholipid stabilized sonoactive microstructures (Sonazoid) and nucleic acids encoding a full length dystrophin protein coupled to a muscle tissue specific promoter sequence, as shown in Table 5 below. The nucleic acid delivery vector administered comprised codon optimized sequences of human dystrophin SEQ ID NO: 3 under a muscle tissue specific promoter of SEQ ID NO: 4 (the combination of which being comprised within the expression cassette of SEQ ID NO: 34). The subject was injected with the phospholipid stabilized sonoactive microstructures (Sonazoid) and nucleic acids via an intravenous infusion into the saphenous vein. Ultrasound energy was applied using a C-16 probe on a GE LOGIQ E10 system in research mode for the acoustic radiation force ultrasound application. An acoustic radiation force ultrasound was applied to the quadriceps of the cyno with the same GE LOGIQ e10 ultrasound system equipped with a C1-6 probe. First, contrast enhanced ultrasound energy was applied to generate an ultrasound image at a mechanical index of 0.4, and a frequency of about 2.5 MHz to confirm the presence of the sonoactive agent in the target muscle tissue. Next, the acoustic radiation force protocol was applied using ultrasound acoustic energy at an ultrasound intensity of about 500 mW/cm2 (ISPTA) (spatial-peak temporal average intensity) at frequency of about 2.3 MHz at a pulse length of 600 μs to disrupt the sonoactive microstructures. Serial bolus injections of approximately 1.0-2.0 mL of sonoactive microstructure and DNA solution were re-administered to the subject and the acoustic radiation force ultrasound was applied until the full dose of nucleic acids were administered, over a treatment duration of about 10 minutes. A rapid loss of ultrasound contrast (due to inertial cavitation of the sonoactive agent) was confirmed following the application of the acoustic radiation force by intermittently switching to contrast enhanced ultrasound mode for imaging.

TABLE 5
In-Vivo Evaluation of Muscle Tissue Specific Vector and Expression of Full-Length
Dystrophin Protein in a NHP Model of Sonoporation to the Skeletal Muscle

| | | DNA | | | | |
|---|---|---|---|---|---|---|
| Group | | Dose | Sonoactive | # of | | Vector/Expression Cassette/Promoter/ |
| (n = 1) | Strain | (ug) | agent | doses | Ultrasound | Transgene |
| 1 | Cyno | 50 | Sonazoid | 1 | Cl-6,~500 | SEQ ID NO: 27 (expression cassette of SEQ |
| | | | | | mW/cm2 Ispta, | ID NO: 34 with muscle tissue specific |
| | | | | | 2.3 MHz, 600 us | promoter of SEQ ID NO: 4 + DMD Codon |
| | | | | | | Optimized SEQ ID NO: 3) |

2 days following the sonoporation treatment, a biopsy was taken from the treated quadriceps for analysis using RNAscope to confirm presence of the transgenic DNA and transgenic mRNA in the target tissue. A control sample biopsy was also taken from the untreated quadriceps for consideration in the analysis. Transverse cryosections (~10 μm thickness) of quadricep muscle that had been collected from non-human primates administered the nucleic acid vector encoding a human transgene delivered via ultrasound mediated nucleic acid deliver were thaw-mounted onto positively charged glass slides and air-dried for 30 minutes at room temperature. Each slide was then secured in a black slide carrier of a motorized auto staining platform and covered with fluorophore-compatible cover-tiles to minimize tissue detachment during processing. Slide records were created in the instrument software by adding a new study and assigning individual slide identifiers. The run method specified a heat-mediated antigen-retrieval step (95° C., 15 min), an endogenous peroxidase block (10 min), and a protease digestion (40° C., 10 min). Probe channel 1 was reserved for a proprietary RNAscope probe targeting the codon-optimized transgene mRNA and transgenic DNA, while channel 2 was allocated to a probe recognizing a housekeeping transcript to verify DNA integrity. All kit reagents supplied with a commercial multiplex fluorescent RNAscope reagent set were registered on the instrument; reagent wands were loaded onto the platform and validated by an automated dip-test. Fluorophores were freshly diluted in tyramide-signal-amplification (TSA) buffer according to manufacturer instructions (typical dilutions:vivid dyes, 1:3 000; near-infra-red dye, 1:125). Target probes were transferred to open reagent tubes placed on an auxiliary wand that was integrated into the reagent platform without interrupting the dip-test cycle. Bulk wash solutions were replenished and waste containers emptied before initiating the staining run. After the slide racks had been lowered and locked, the auto stainer executed the programmed protocol, which included: hybridization of target probes at 42° C. for 2 h; six proprietary amplification steps; and sequential TSA-mediated deposition of distinct fluorophores for channels 1 and 2. A final bake at 60° C. for 30 min ensured covalent coupling of fluorophores to the amplification scaffold. Upon run completion, slide racks were released, cover-tiles were removed, and the sections were washed three times in running tap water, immersed for 2 min in 0.01% ammonium hydroxide to suppress non-specific background, rinsed twice again, and dipped in deionized water. Slides were dried in a 60° C. oven for 10 min and permanently mounted in an antifade medium containing DAPI to stain nuclei. Fluorescence microscopy (20× objective) revealed discrete cytoplasmic puncta corresponding to the transgene-specific transcript in a subset of myofibers, whereas the housekeeping-gene probe produced uniform staining, confirming assay integrity. Negative-control sections treated with a bacterial dapB probe displayed≤1 punctum per field, establishing specificity. The RNAscope protocol enabled robust in-situ detection of the target transgenic DNA and mRNA within non-human-primate quadriceps muscle.

Biopsy samples were further analyzed for DMD DNA and mRNA in the NHP muscle tissue samples to quantify percentage of positive muscle fibers in treated tissue using HALO (Indica Labs) by staining quadriceps samples from subjects with a probe which binds hDMD DNA and mRNA. Using HALO and HALO AI v4.0.5107.407 muscle tissue images were uploaded and opened for analysis. Tissue boundaries were annotated using the Flood-fill tool, with refinements made via the Pen and Brush tools. Debris and unwanted regions were excluded. Nuclear segmentation was performed using Indica Labs' pre-trained “Nuc Seg BF” classifier, with real-time tuning for optimization. HALO AI classifiers (RandomForest, MiniNet, or DenseNet) were applied based on the complexity of the regions of interest (ROIs). For analysis, the “Indica Labs-ISH v4.2.3” module was selected, and stain settings were configured for a single probe. Nuclear and probe stains were calibrated by selecting representative nuclei and puncta, while debris exclusion settings were applied. Since singleplex staining was used, phenotype and AI phenotyper settings were left blank. Real-time tuning validated puncta and nuclear recognition, with adjustments were made to segmentation aggressiveness, probe size, and optical density thresholds as needed. Cell-based analysis was performed by enabling “Detect Cells” and applying the nuclear segmentation classifier. Basescope criteria were set for expression quantification. The ROI classifier and relevant classes were selected, and analysis was executed on the designated tissue regions. Greater than 50% fiber membrane had to contain hDMD puncta to be counted as positive using this analysis.

### Results

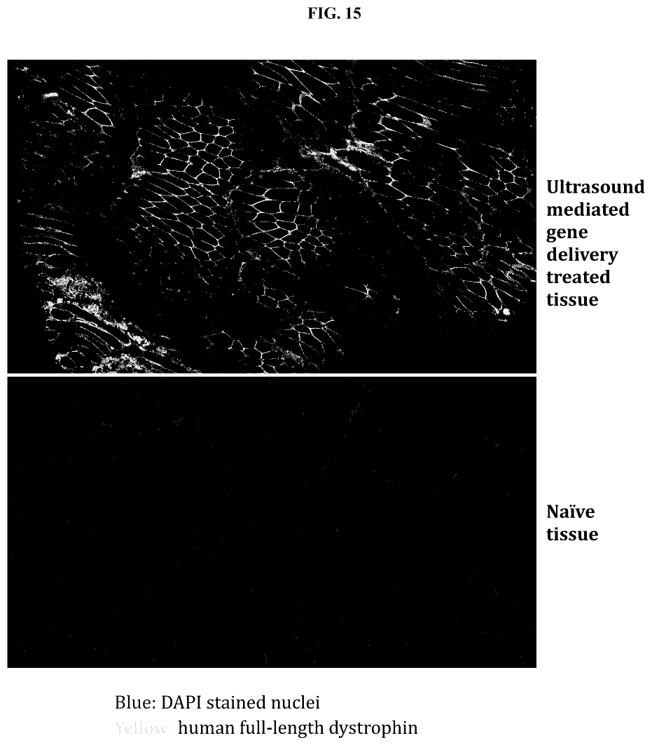

Results of the RNAscope analysis is shown in FIG. 15, in which it is observed that the naïve tissue sample shows DAPI stained nuclei throughout the sample but no yellow staining indicating a lack of presence of the dystrophin transgenic DMD nucleic acids; while the treated tissue sample treated with the ultrasound mediated nucleic acid (gene) delivery showed DAPI stained nuclei throughout the sample and extensive yellow staining along the length of a muscle fibers in the muscle tissue about the sarcolemma of a muscle fibers on the periphery of the cellular membrane of the myocytes, indicating extensive presence of the dystrophin transgenic DMD nucleic acids throughout the treated tissue.

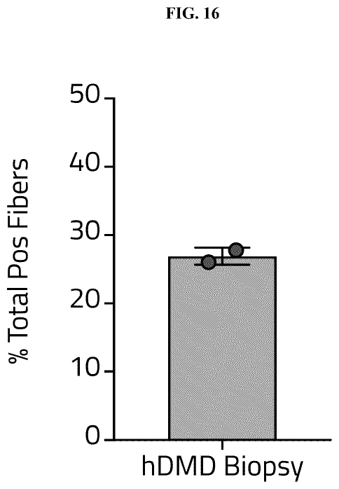

Results of the HALO analysis shown in FIG. 16, in which it is observed that the treated tissue sample exhibited approximately 28% of muscle fibers which were positive for transgenic DMD nucleic acids, with each positive fiber having greater than 50% fiber membrane positive for hDMD puncta.

### Example 7: In-Vivo Evaluation of Muscle Tissue Specific Vector and Expression in a Murine Model of Sonoporation to the Skeletal Muscle

In this example, nucleic acid delivery vectors with expression cassettes comprising promoter sequences which were optimized for expression in skeletal myocytes were evaluated in a murine model of ultrasound mediated nucleic acid delivery to the skeletal muscle (quadriceps).

### Experimental Animals and Protocol

One group of three RAG 2 mice are evaluated for long term expression of a FLUC reporter over 275 days. Prior to the experiment, mice were implanted with a jugular vein catheter though which the sonoactive agent and the DNA solution was administered. Optison (human serum albumin stabilized perflutran core microbubbles) was used as the ultrasound contrast agent in this experiment. Mice were administered three sonoporation treatments, with the second and third treatments given 24 hours after the preceding treatment. Mice were administered 250 μg of nucleic acids comprising pDNA comprising a sequence of SEQ ID NO: 30 (muscle tissue specific promoter (SEQ ID NO: 4)+FLUC reporter).

The mice received application of ultrasound energy following administration of 50 uL of the sonoactive agent and one third of the DNA payload in each treatment session. In a treatment session, the left leg of each animal was placed in a heated water bath at 38° C. for 2 minutes prior to applying ultrasound using a L8-18 probe on a General Electric LOGIQ E9 system in Elasto mode applying an acoustic radiation force at a frequency of about 2.5 MHz at a mechanical index of 1.4 at a 100% push output at an ultrasound intensity of at least 200 mW/cm2 (ISPTA) for about 10 sec on, 20 sec off, for a total of 60 sec (2 cycles) of ultrasound application over the left quadriceps region of each mouse. A second identical treatment session was applied at 24 hours following the first treatment session, and a third identical treatment session was applied at 24 hours after the second treatment session. IVIS measurements were taken approximately weekly to monitor ongoing expression of the FLUC reporter protein.

### Results

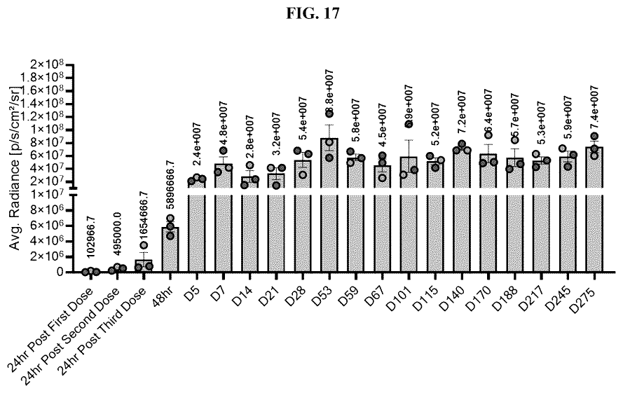

Results are shown in FIG. 17, in which it is observed that expression in the treated muscle tissue was maintained with IVIS measurements exceeding 2.4 E7 for the duration of the study, with a level of 7.4 E7 observed at day 275 of the study.

### Example 8: In-Vivo Evaluation of Muscle Tissue Specific Vector and Expression in a NHP Model of Sonoporation to the Heart and Diaphragm

In this example, nucleic acid delivery vectors with expression cassettes comprising promoter sequences which were optimized for expression in skeletal myocytes cells were evaluated in a non-human primate model of ultrasound mediated nucleic acid delivery to the heart and diaphragm.

### Experimental Animals and Protocol

A first subject was one male cynomolgus macaque (cyno), which was evaluated for nucleic acid delivery in the heart. The subject was infused with phospholipid stabilized sonoactive microstructures (Sonazoid), and nucleic acids encoding a EGFP reporter protein. The second subject was one male cynomolgus macaque (cyno), which was evaluated for nucleic acid delivery in the diaphragm. The second subject was infused with phospholipid stabilized sonoactive microstructures (Sonazoid), and nucleic acids encoding a EGFP reporter protein. Identical ultrasound procedures and nucleic acid administration techniques were applied to each subject. Table 6 below summarizes the experimental animals and protocol.

The subjects were injected with the phospholipid stabilized sonoactive microstructures (Sonazoid), and nucleic acids via an intravenous infusion into the saphenous vein. Ultrasound energy was applied using a C-16 probe on a GE LOGIQ E10 system in research mode for the acoustic radiation force ultrasound application. An acoustic radiation force ultrasound was applied to the heart or diaphragm of the cyno with the same GE LOGIQ e10 ultrasound system equipped with a C1-6 probe. First, contrast enhanced ultrasound energy was applied to generate an ultrasound image at a mechanical index of 0.4, and a frequency of about 2.5 MHz to confirm the presence of the sonoactive agent in the target muscle tissue. Next, the acoustic radiation force protocol was applied using ultrasound acoustic energy at an ultrasound intensity of about 500 mW/cm2 (ISPTA) (spatial-peak temporal average intensity) at frequency of about 2.3 MHz at a pulse length of 600 μs to disrupt the sonoactive microstructures. Serial bolus injections of approximately 1.0-2.0 mL of sonoactive microstructure and DNA solution were re-administered to the subject and the acoustic radiation force ultrasound was applied until the full dose of nucleic acids were administered, over a treatment duration of about 10 minutes. A rapid loss of ultrasound contrast (due to inertial cavitation of the sonoactive agent) was confirmed following the application of the acoustic radiation force by intermittently switching to contrast enhanced ultrasound mode for imaging.

TABLE 6
In-Vivo Evaluation of Muscle Tissue Specific Vector and Expression
in a NHP Model of Sonoporation to the Heart and Diaphragm

| | | DNA | | | | |
|---|---|---|---|---|---|---|
| Group | Strain | Dose | Sonoactive | # of | | |
| (n = 1) | (organ) | (ug) | agent | doses | Ultrasound | Vector/Promoter |
| 1 | Cyno | 50 | Sonazoid | 1 | Cl-6,~500 | SEQ ID NO: 30(NP-MHCK7-hBGi- |
| | (heart) | | | | mW/cm2 Ispta, | EGFP-P2A-Fluc-WPRE3-DTS) |
| | | | | | 2.3 MHz, 600 us | comprising the promoter MHCK7 |
| | | | | | | sequence of SEQ ID NO: 4 |
| 1 | Cyno | 50 | Sonazoid | 1 | Cl-6, ~500 | SEQ ID NO: 30(NP-MHCK7-hBGi- |
| | (diaphragm) | | | | mW/cm2 Ispta, | EGFP-P2A-Fluc-WPRE3-DTS) |
| | | | | | 2.3 MHz, 600 us | comprising the promoter MHCK7 |
| | | | | | | sequence of SEQ ID NO: 4 |

Necropsy was performed 7 hours following administration of the sonoporation treatment. Samples were collected from the treated heart and diaphragm. Tissue that had been collected from non-human primates administered the nucleic acid vector encoding a human transgene delivered via ultrasound mediated nucleic acid deliver were thaw-mounted onto positively charged glass slides and air-dried for 30 minutes at room temperature. Each slide was then secured in a black slide carrier of a motorized auto staining platform and covered with fluorophore-compatible cover-tiles to minimize tissue detachment during processing. Slide records were created in the instrument software by adding a new study and assigning individual slide identifiers. The run method specified a heat-mediated antigen-retrieval step (95° C., 15 min), an endogenous peroxidase block (10 min), and a protease digestion (40° C., 10 min). Probe channel 1 was reserved for a proprietary RNAscope probe targeting the codon-optimized transgene DNA and mRNA, while channel 2 was allocated to a probe recognizing a housekeeping transcript to verify DNA integrity. All kit reagents supplied with a commercial multiplex fluorescent RNAscope reagent set were registered on the instrument; reagent wands were loaded onto the platform and validated by an automated dip-test. Fluorophores were freshly diluted in tyramide-signal-amplification (TSA) buffer according to manufacturer instructions (typical dilutions:vivid dyes, 1:3 000; near-infra-red dye, 1:125). Target probes were transferred to open reagent tubes placed on an auxiliary wand that was integrated into the reagent platform without interrupting the dip-test cycle. Bulk wash solutions were replenished and waste containers emptied before initiating the staining run. After the slide racks had been lowered and locked, the auto stainer executed the programmed protocol, which included: hybridization of target probes at 42° C. for 2 h; six proprietary amplification steps; and sequential TSA-mediated deposition of distinct fluorophores for channels 1 and 2. A final bake at 60° C. for 30 min ensured covalent coupling of fluorophores to the amplification scaffold. Upon run completion, slide racks were released, cover-tiles were removed, and the sections were washed three times in running tap water, immersed for 2 min in 0.01% ammonium hydroxide to suppress non-specific background, rinsed twice again, and dipped in deionized water. Slides were dried in a 60° C. oven for 10 min and permanently mounted in an antifade medium containing DAPI to stain nuclei. Fluorescence microscopy (20× objective) revealed discrete cytoplasmic puncta corresponding to the transgene-specific transcript in a subset of myofibers, whereas the housekeeping-gene probe produced uniform staining, confirming assay integrity. Negative-control sections treated with a bacterial dapB probe displayed≤1 punctum per field, establishing specificity. The RNAscope protocol enabled robust in-situ detection of the target transgenic nucleic acid within non-human-primate tissues.

Biopsy samples were further analyzed for transgene DNA and mRNA in the NHP tissue samples to quantify percentage of positive muscle fibers in treated tissue using HALO (Indica Labs) by staining quadriceps samples from subjects with a probe which binds hDMD DNA and mRNA. Using HALO and HALO AI v4.0.5107.407 muscle tissue images were uploaded and opened for analysis. Tissue boundaries were annotated using the Flood-fill tool, with refinements made via the Pen and Brush tools. Debris and unwanted regions were excluded. Nuclear segmentation was performed using Indica Labs' pre-trained “Nuc Seg BF” classifier, with real-time tuning for optimization. HALO AI classifiers (RandomForest, MiniNet, or DenseNet) were applied based on the complexity of the regions of interest (ROIs). For analysis, the “Indica Labs-ISH v4.2.3” module was selected, and stain settings were configured for a single probe. Nuclear and probe stains were calibrated by selecting representative nuclei and puncta, while debris exclusion settings were applied. Since singleplex staining was used, phenotype and AI phenotyper settings were left blank. Real-time tuning validated puncta and nuclear recognition, with adjustments made to segmentation aggressiveness, probe size, and optical density thresholds as needed. Cell-based analysis was performed by enabling “Detect Cells” and applying the nuclear segmentation classifier. Basescope criteria were set for expression quantification. The ROI classifier and relevant classes were selected, and analysis was executed on the designated tissue regions. Greater than 50% fiber membrane must contain hDMD puncta to be counted as positive using this analysis.

Samples were evaluated using digital droplet polymer chain reaction (ddPCR) to assess copy number per diploid genome (CN/DG) levels of DNA payload delivered to the cells, quantified with respect to a reference gene. The procedure began by preparing the Master Mix (MM) for each reaction. First, the ddPCR Supermix for probes (no dUTP) was thawed and vortexed for at least 30 seconds. For each primer/probe set, 11 μL ddPCR Supermix, 1.1 μL primer/probe mix, 0.275 μL HindIII enzyme, and water were combined to bring the final reaction volume to 18 μL. For single-plex reactions, 5.626 μL of water was added, while for duplex reactions, 4.525 μL was added. The Master Mix was calculated with a correction factor of 1.1 to account for pipetting error. After preparation, 18 μL of the Master Mix was dispensed into individual wells of a 96-well PCR plate, which was then set aside at room temperature. DNA was extracted in duplicates from 15-30 mg adipose tissue samples using QIAGEN DNeasy kit. DNA were diluted to 0.025, 0.075, and 0.25 ng/uL using nuclease-free water, and 4 μL of the diluted genomic DNA (gDNA) or ddH2O (for negative controls) was added to each well according to the plate map. The plate was then sealed with a pierceable foil seal, ensuring the red stripe faced up at the top, and vortexed for 2 minutes at room temperature. Following this, the plate was spun down at 100-300 rcf for 1 minute to remove bubbles. The sealed plate was placed in the Automated Droplet Generator (ADG), ensuring that the destination plate was on a cooling block, DG32 cartridges, ddPCR tips (with lids removed), and an empty waste container were properly positioned. The droplet generation process was initiated, and the corresponding ddPCR program was selected on the thermal cycler, which was paused while waiting for the droplet generation to finish. Once the droplet generation was completed, the 96-well plate was removed from the cooling block, resealed with a pierceable foil seal, and transferred to the thermal cycler, where the cycling process was resumed. The cooling block was returned to −20° C. in an upside-down position for future use. Thermal cycling was performed based on the ddPCR program, which typically included steps such as denaturation, annealing, and extension, although specific conditions depended on the primer/probe set used. After thermal cycling, the plate was transferred to the Droplet Reader. In the ddPCR software, the appropriate Supermix and fluorophores (e.g., FAM, VIC, HEX) were selected before initiating the droplet reading process. Once the droplet reading was completed, the results provided the quantification of plasmid DNA copy number per diploid genome, as well as its relative abundance to the reference gene. The software generated data based on the fluorescence signals detected in the droplets, enabling accurate measurement of the target gene copy numbers.

### Results

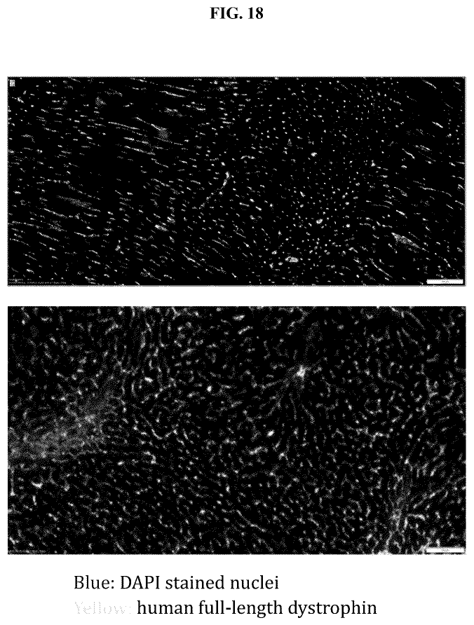

Results of the RNAscope analysis are shown in FIG. 18, in which the treated NHP heart tissues (top panel) are observed to exhibit delivery of transgenic DNA expression of transgene RNA (yellow staining) and treated diaphragm tissues (bottom panel) are observed to exhibit expression of transgene DNA and RNA (yellow staining) throughout the treated tissue.

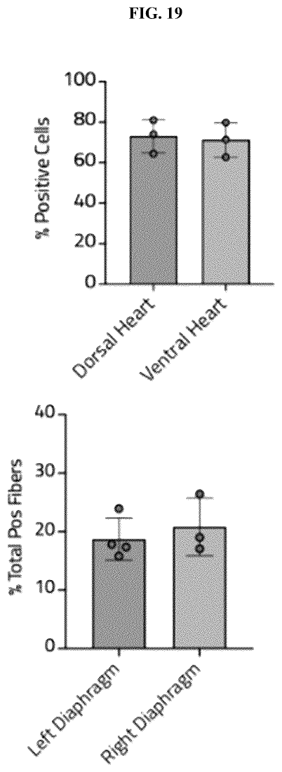

Results of the HALO analysis are shown in FIG. 19, in which it is observed that the treated cardiac tissue sample (top graph) exhibited approximately 70% of muscle fibers which were positive for the transgene in both the dorsal and ventral regions of the heart, and treated diaphragm samples (lower graph) exhibited approximately 20% of muscle fibers which were positive for the transgene in both the left and right regions of the diaphragm.

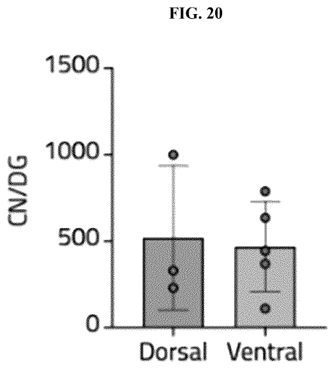

Results of the copy number analysis are shown in FIG. 20, in which it is observed that an average of 500 copies per diploid genome (CN/DG) of transgenic DNA are delivered to each cell in treated cardiac tissue samples, in both the dorsal and ventral regions of the heart.

### Example 9: In-Vivo Evaluation of Muscle Tissue Specific Vector and Expression of Full-Length Dystrophin Protein in a NHP Model of Sonoporation to the Skeletal Muscle

In this example, nucleic acid delivery vectors with expression cassettes comprising promoter sequences which are optimized for expression in skeletal myocytes cells were evaluated in a non-human primate model of ultrasound mediated nucleic acid delivery to the skeletal muscle (quadriceps).

### Experimental Animals and Protocol

As summarized in Table 7 below, the subject was one male cynomolgus macaque (cyno). the subject was infused with phospholipid stabilized sonoactive microstructures (Sonazoid), and nucleic acids encoding a full length monkey dystrophin protein coupled to a muscle tissue specific promoter sequence. The subject was injected with the phospholipid stabilized sonoactive microstructures (Sonazoid), and nucleic acids via an intravenous infusion into the saphenous vein. Ultrasound energy was applied using a C-16 probe on a GE LOGIQ E10 system in research mode for the acoustic radiation force ultrasound application. An acoustic radiation force ultrasound was applied to the quadriceps of the cyno with the same GE LOGIQ e10 ultrasound system equipped with a C1-6 probe. First, contrast enhanced ultrasound energy was applied to generate an ultrasound image at a mechanical index of 0.4, and a frequency of about 2.5 MHz to confirm the presence of the sonoactive agent in the target muscle tissue. Next, the acoustic radiation force protocol was applied using ultrasound acoustic energy at an ultrasound intensity of about 500 mW/cm2 (ISPTA) (spatial-peak temporal average intensity) at frequency of about 2.3 MHz at a pulse length of 600 μs to disrupt the sonoactive microstructures. Serial bolus injections of approximately 1.0-2.0 mL of sonoactive microstructure and DNA solution were re-administered to the subject and the acoustic radiation force ultrasound was applied until the full dose of nucleic acids were administered, over a treatment duration of about 10 minutes. A rapid loss of ultrasound contrast (due to inertial cavitation of the sonoactive agent) was confirmed following the application of the acoustic radiation force by intermittently switching to contrast enhanced ultrasound mode for imaging.

TABLE 7
In-Vivo Evaluation of Muscle Tissue Specific Vector and Expression of Full-Length
Dystrophin Protein in a NHP Model of Sonoporation to the Skeletal Muscle

| | | DNA | | | | |
|---|---|---|---|---|---|---|
| Group | | Dose | Sonoactive | # of | | |
| (n = 1) | Strain | (ug) | agent | doses | Ultrasound | Vector/Promoter |
| 1 | Cyno | 5 | Sonazoid | 1 | Cl-6, ~500 | SEQ ID NO: 33 (vector with muscle tissue |
| | | | | | mW/cm2 Ispta, | specific promoter (SEQ ID NO 4) + monkey |
| | | | | | 2.3 MHz, 600 us | DMD) |

5 days following treatment, a biopsy was taken from the treated quadriceps for analysis. Treated muscle tissue was evaluated for DMD expression using western blot analysis. Western blot analysis was performed as described in Example 1. Samples were further analyzed for total dystrophin expression normalized to total protein expression and were compared to a control which was a quadriceps biopsy taken from an untreated naive control animal.

### Results

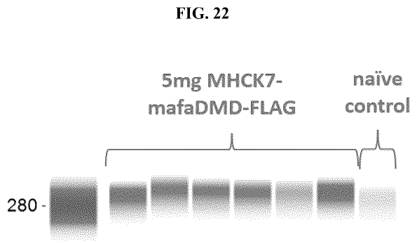

Results of the western blot assay are shown in FIG. 22, where binding of the antibody at a level which exceeds the negative (naïve) control is observed in all treated samples.

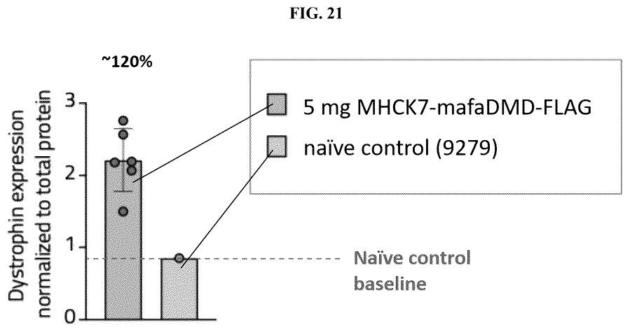

Results of the protein normalization assay are shown in FIG. 21, in which the treated muscle tissue exhibited approximately 220% of normal dystrophin levels, representing a 120% increase over the baseline dystrophin expression expected in a naive animal.

### Example 10: In-Vivo Evaluation of Muscle Tissue Specific Vector and Expression of Full-Length Dystrophin Protein in a NHP Model of Sonoporation to the Skeletal Muscle

In this example, nucleic acid delivery vectors with expression cassettes comprising promoter sequences which were optimized for expression in skeletal myocytes cells, and an expression cassette combining said promoter sequences with codon optimized DMD sequences were evaluated in a non-human primate model of ultrasound mediated nucleic acid delivery to the skeletal muscle (quadriceps). The vector further encoded an HA tag to enhance detection of the expressed protein product.

### Experimental Animals and Protocol

As summarized in Table 8 below, one male cynomolgus macaque (cyno), the subject, was infused with phospholipid stabilized sonoactive microstructures (Sonazoid) and nucleic acids encoding a full length monkey dystrophin protein coupled to a muscle tissue specific promoter sequence. The subject was injected with the phospholipid stabilized sonoactive microstructures (Sonazoid), and nucleic acids via an intravenous infusion into the saphenous vein. Prior to the infusion and administration of ultrasound, a heating pad was applied to the quadricep to be treated for 2-minutes immediately preceding the infusion. Ultrasound energy was applied using a C-16 probe on a GE LOGIQ E10 system in research mode for the acoustic radiation force ultrasound application. An acoustic radiation force ultrasound was applied to the quadricep of the cyno with the same GE LOGIQ e10 ultrasound system equipped with a C1-6 probe. First, contrast enhanced ultrasound energy was applied to generate an ultrasound image at a mechanical index of 0.09, and a frequency of about 2.5 MHz to confirm the presence of the sonoactive agent in the target muscle tissue. Next, the acoustic radiation force protocol was applied using ultrasound acoustic energy at an ultrasound intensity of about 500 mW/cm2 (ISPTA) (spatial-peak temporal average intensity) at frequency of about 2.5 MHz at a pulse length of 600 μs to disrupt the sonoactive microstructures. Serial bolus injections of approximately 1.0-2.0 mL of sonoactive microstructure and DNA solution were re-administered to the subject and the acoustic radiation force ultrasound was applied until the full dose of nucleic acids were administered, over a treatment duration of about 10 minutes. A rapid loss of ultrasound contrast (due to inertial cavitation of the sonoactive agent) was confirmed following the application of the acoustic radiation force by intermittently switching to contrast enhanced ultrasound mode for imaging.

TABLE 8
In-Vivo Evaluation of Muscle Tissue Specific Vector and Expression of Full-Length
Dystrophin Protein in a NHP Model of Sonoporation to the Skeletal Muscle

| | | DNA | | | | |
|---|---|---|---|---|---|---|
| Group | | Dose | Sonoactive | # of | | |
| (n = 1) | Strain | (ug) | agent | doses | Ultrasound | Expression Cassette/Promoter/Transgene |
| 1 | Cyno | 5 | Sonazoid | 1 | Cl-6, ~500 | SEQ ID NO: 34 (vector with muscle tissue |
| | | | | | mW/cm2 Ispta, | specific promoter (SEQ ID NO 4) + DMD |
| | | | | | 2.3 MHz, 600 us | Codon Optimized SEQ ID NO: 3) |

7 hours following treatment, necropsy was performed and a sample was taken from the treated quadriceps for analysis. Samples were analyzed using RNAscope to confirm presence of the transgenic DNA in the target tissue, and were further analyzed for DMD DNA and mRNA in the NHP muscle tissue samples to quantify percentage of positive muscle fibers in treated tissue using HALO (Indica Labs) by staining quadriceps samples from subjects with a probe that binds hDMD, as described in Example 6. Samples were also evaluated using digital droplet polymer chain reaction (ddPCR) to assess copy number per diploid genome (CN/DG) levels of DNA payload delivered to the cells, quantified with respect to a reference gene, as described in Example 8.

### Results

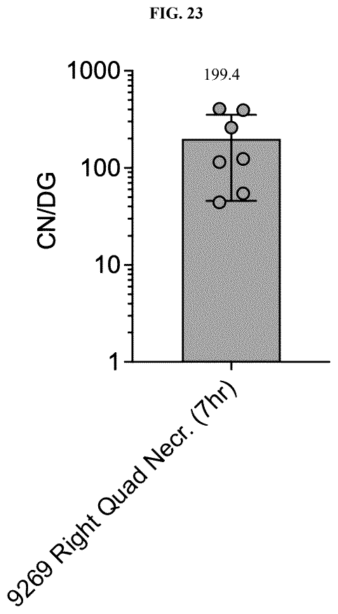

Results of the copy number analysis are shown in FIG. 23, in which it is observed that an average of 199.4 copies per diploid genome (CN/DG) of transgenic DNA are delivered to each cell in treated muscle tissue.

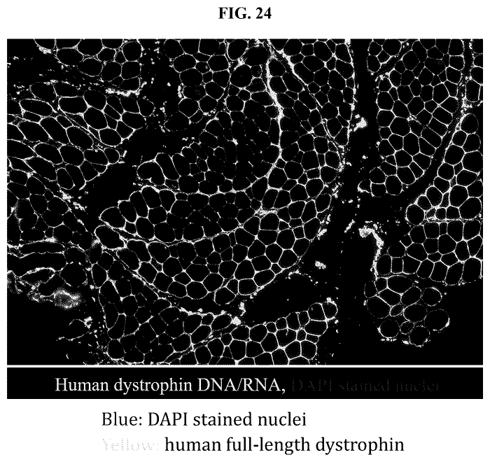

Results of the RNAscope analysis is shown in FIG. 24, in which it is observed that the treated tissue sample had DAPI stained nuclei throughout the sample and extensive yellow staining along the length of muscle fibers in the muscle tissue, specifically about the sarcolemma of the muscle fibers, and on the periphery of the cellular membrane of the myocytes, indicating extensive presence of the dystrophin transgenic DMD nucleic acids throughout the treated tissue.

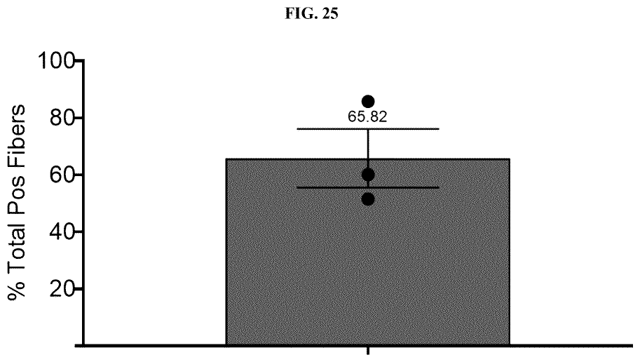

Results of the HALO analysis shown in FIG. 25, in which it is observed that the treated tissue sample exhibits approximately 65% of muscle fibers which are positive for transgenic DMD nucleic acids, with each positive fiber having greater than 50% fiber membrane positive for hDMD puncta.

### Example 11: Evaluation of Nucleic Acid Delivery Vectors in a Murine Model of Ultrasound Mediated Nucleic Acid Transfection to the Kidney Evaluating a Ubiquitous Promoter Sequence in the Kidney

In this example, nucleic acid delivery vectors with expression cassettes comprising promoter sequences which were optimized for expression in multiple cell types were evaluated in a murine model of ultrasound mediated nucleic acid delivery to the kidney to evaluate the efficacy of a ubiquitous promoter sequence disclosed herein in the kidney.

### Experimental Animals and Protocol

There are a total of 3 experimental groups of six RAG2 mice each of which varies the dosage of the promoter sequence operably coupled to the gene encoding the reporter protein were utilized in the experiment. Each group of mice received an intravenous infusion through an implanted jugular vein catheter of a composition comprising the sonoactive agent and the DNA payload. Definity RT was used as the sonoactive agent in this experiment. Each group received a dose of the nucleic acid delivery vector followed by application of ultrasound in a treatment session, and was administered a total of three treatment session each treatment session, 48 hours apart. The experimental groups are summarized in Table 9 below.

TABLE 9
Evaluation of Nucleic Acid Delivery Vectors in a Murine Model
of Ultrasound Mediated Nucleic Acid Transfection to the Kidney
Evaluating a Ubiquitous Promoter Sequence in the Kidney

| | | DNA | | | |
|---|---|---|---|---|---|
| Group | | Dose | | # of | |
| (n = 6) | Strain | (ug) | MB | doses | Vector |
| 1 | Rag2 | 100 | Definity | 3 | SEQ ID NO: 42 |
| | | | RT | | (comprising CAG promoter |
| | | | | | of SEQ ID NO: 5) |
| 2 | Rag2 | 100 | Definity | 3 | SEQ ID NO: 43 |
| | | | RT | | (comprising CBA promoter |
| | | | | | of SEQ ID NO: 37) |
| 3 | Rag2 | 100 | Definity | 3 | SEQ ID NO: 45 |
| | | | RT | | (comprising NPHSI promoter |
| | | | | | of SEQ ID NO: 44) |

Each experimental group received identical application of ultrasound energy following administration of the sonoactive agent and DNA payload. Using a L8-18 probe on a General Electric LOGIQ E9 system in Elasto mode, ultrasound was applied at a frequency of about 2.5 MHz at a mechanical index of 1.7 at a 100% push output applying a 300 μs push pulse with a pulse repetition frequency of 0.5 Hz at an ultrasound intensity of at least 200 mW/cm2 (ISPTA) for about 40 seconds of ultrasound application over the left kidney region of each mouse.

3 days following the final sonoporation treatment, the mice were injected with D-Luciferin, potassium salt and imaged using IVIS.

### Results

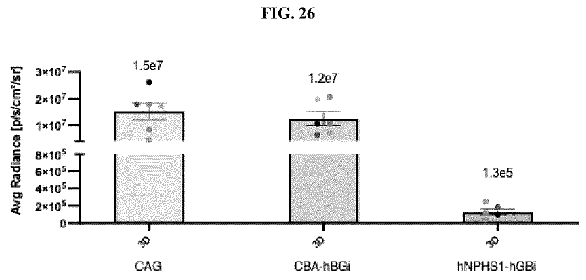

The results of this experiment are illustrated in FIG. 26. In FIG. 26, it is shown that mice treated with the vector of SEQ ID NO: 43 (comprising CBA promoter of SEQ ID NO: 37) exhibited average radiance of about 1.2 E7 p/s/cm2/sr, mice treated with the vector of SEQ ID NO: 42 (comprising CBA promoter of SEQ ID NO: 5) exhibited average radiance of about 1.5 E7 p/s/cm2/sr, and mice treated with the vector of SEQ ID NO: 45 (comprising NPHS1 promoter of SEQ ID NO: 37) exhibited average radiance of about 1.3 E5 p/s/cm2/sr. Of note is that mice treated with the vector of SEQ ID NO: 43 (comprising the ubiquitous CBA promoter of SEQ ID NO: 37) exhibited gene expression consistent with mice treated with the vector of SEQ ID NO: 42 comprising the well-known and highly effective ubiquitous CAG promoter, and exhibited gene expression which significantly out-performed mice treated with the vector of SEQ ID NO: 45 comprising the kidney tissue specific NPHS1 promoter.

### Example 12: Evaluation of Nucleic Acid Delivery Vectors in a Murine Model of Ultrasound Mediated Nucleic Acid Transfection to the Muscle Evaluating a Ubiquitous Promoter Sequence in the Muscle

In this example, nucleic acid delivery vectors with expression cassettes comprising the ubiquitous CBA promoter sequences disclosed herein were evaluated in a murine model of ultrasound mediated nucleic acid delivery to the skeletal muscle (quadriceps).

### Experimental Animals and Protocol

Five groups of RAG2 mice were evaluated as summarized in Table 10 below. Prior to the experiment, mice were implanted with a jugular vein catheter though which the sonoactive agent and the DNA solution was administered. The experimental groups were set forth below. In brief, Group 1 was a no-ultrasound control administered only the vector and the sonoactive agent, Group 2-3 were administered vectors comprising a gene encoding a fluorescent reporter protein (FLUC) under either a ubiquitous promoter of the present disclosure (SEQ ID NO: 37) or a muscle tissue specific promoter (SEQ ID NO: 10) with administration of a sonoactive agent and ultrasound, and Groups 4-5 were administered human dystrophin under either a ubiquitous or a muscle tissue specific promoter (SEQ ID NO: 10) with administration of a sonoactive agent and ultrasound. Optison (human serum albumin stabilized perflutran core microbubbles) was used as the ultrasound contrast agent in this experiment.

TABLE 10
Evaluation of Nucleic Acid Delivery Vectors in a Murine Model of Ultrasound Mediated Nucleic
Acid Transfection to the Muscle Evaluating a Ubiquitous Promoter Sequence in the Muscle

| | | DNA | | | | |
|---|---|---|---|---|---|---|
| Group | | Dose | Sonoactive | # of | | |
| (n = 3) | Strain | (ug) | agent | doses | Ultrasound | Vector |
| 1 | Rag2 | 250 | Optison | 3 | None | SEQ ID NO: 47 (SEQ ID NO: 37 |
| | | | | | | ubiquitous promoter + FLUC reporter) |
| 2 | Rag2 | 250 | Optison | 3 | L8-18, MI 1.4, 10 | SEQ ID NO: 47 (SEQ ID NO: 37 |
| | | | | | sec on, 20 sec off, | ubiquitous promoter + FLUC reporter) |
| | | | | | 60 sec total on | |
| 3 | Rag2 | 250 | Optison | 3 | L8-18, MI 1.4, 10 | SEQ ID NO: 48 (muscle tissue specific |
| | | | | | sec on, 20 sec off, | promoter + FLUC reporter) |
| | | | | | 60 sec total on | |
| 4 | Rag2 | 250 | Optison | 3 | L8-18, MI 1.4, 10 | SEQ ID NO: 49 (SEQ ID NO: 37 |
| | | | | | sec on, 20 sec off, | ubiquitous promoter + DMD) |
| | | | | | 60 sec total on | |
| 5 | Rag2 | 250 | Optison | 3 | L8-18, MI 1.4, 10 | SEQ ID NO: 50 (muscle tissue specific |
| | | | | | sec on, 20 sec off, | promoter + DMD) |
| | | | | | 60 sec total on | |

The mice received application of ultrasound energy following administration of 50 uL of the sonoactive agent and one third of the DNA payload in each treatment session. In a treatment session, the left leg of each animal was placed in a heated water bath at 38° C. for 2 minutes prior to applying ultrasound using a L8-18 probe on a General Electric LOGIQ E9 system in Elasto mode applying an acoustic radiation force at a frequency of about 2.5 MHz at a mechanical index of 1.4 at a 100% push output at an ultrasound intensity of at least 200 mW/cm2 (ISPTA) for about 10 sec on, 20 sec off, for a total of 60 sec of ultrasound application over the left quadriceps region of each mouse. A second identical treatment session was applied at 24 hours after the first treatment session, and a third identical treatment session was applied at 24 hours after the second treatment session.

For Groups 1-3, the mice were injected with D-Luciferin, potassium salt and imaged using IVIS 1 day following the first treatment session; 1 day following the second treatment session; and 1, 2 and 6 days following the third treatment session, and

For Groups 4-5, mice were sacrificed 4 days following the third treatment session. Tissue samples were collected, placed into a sterile tube, and snap frozen using liquid N₂. Tissue samples were directly transferred to −80 C storage. Capillary western blot analysis was performed as described in Example 1, with the addition of human skeletal muscle lysate was normalized to 2 ug/ul, then diluted to create standards at 100, 50, 25, 5, 2, and 1%. A 2 μg/uL concentration was the selected concentration for the western blot analysis. Samples were then diluted to 2 μg/uL and compared with human skeletal muscle lysate standards.

### Results

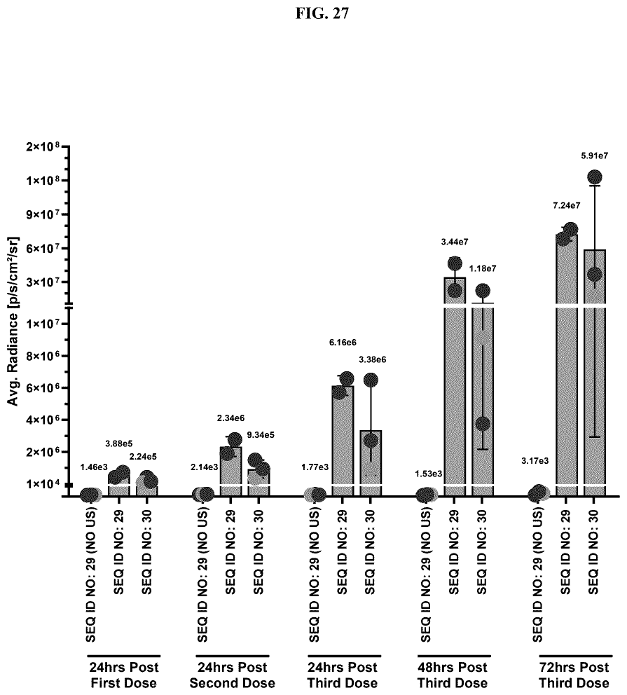

Results for Groups 1-3 are shown in FIG. 27, in which it is shown that the negative control, group 1, exhibited only background levels of average radiance at all time points; Group 2, administered the vector of SEQ ID NO: 47 (ubiquitous promoter+FLUC reporter), exhibited average radiance levels of about 2.3 E6 p/s/cm2/sr at 24 hours following the second treatment, average radiance levels of about 6.2 E6 p/s/cm2/sr at 24 hours following the third treatment, average radiance levels of about 3.44 E7 p/s/cm2/sr at 48 hours following the third treatment, and average radiance levels of about 7.2 E7 p/s/cm2/sr at 72 hours following the third treatment; and Group 3, administered the vector of SEQ ID NO: 48 (muscle tissue specific promoter+FLUC reporter), exhibited average radiance levels of about 9.3 E5 p/s/cm2/sr at 24 hours following the second treatment, average radiance levels of about 3.4 E6 p/s/cm2/sr at 24 hours following the third treatment, average radiance levels of about 1.2 E7 p/s/cm2/sr at 48 hours following the third treatment, and average radiance levels of about 5.9 E7 p/s/cm2/sr at 72 hours following the third treatment. Of note is that mice treated with the vectors comprising the SEQ ID NO: 37 ubiquitous promoter exhibited significantly increased gene expression as compared to groups treated with muscle tissue specific promoters.

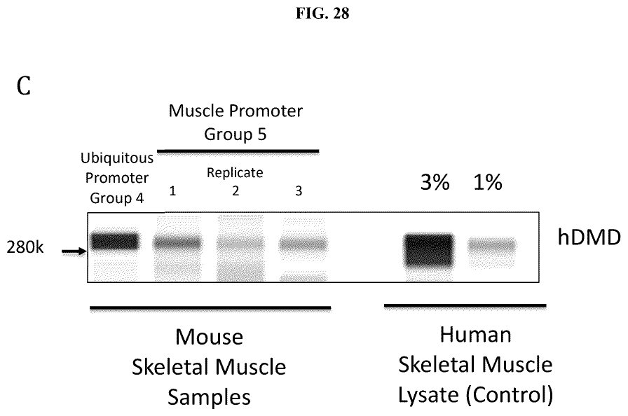

Results of the capillary western blot are shown in FIG. 28, in which samples are diluted to 2 μg/uL and compared with human skeletal muscle lysate standards. In FIG. 28, it is observed that Group 4, administered the vector of SEQ ID NO: 49 comprising a ubiquitous promoter coupled to a dystrophin transgene, exhibited strong expression of the full length dystrophin protein consistent with the 3% human skeletal muscle lysate standard, while Group 5, administered the vector of SEQ ID NO: 50 comprising a muscle tissue specific promoter coupled to a dystrophin transgene, exhibited expression of the full length dystrophin protein consistent with the 1% human skeletal muscle lysate standard. Of note is that mice treated with the vectors comprising the SEQ ID NO: 37 ubiquitous promoter exhibited significantly increased gene expression as compared to groups treated with muscle tissue specific promoters.

### Example 13: Evaluation of Nucleic Acid Delivery Vectors in a Murine Model of Ultrasound Mediated Nucleic Acid Transfection to the Liver Evaluating a Ubiquitous Promoter Sequence in the Liver

In this example, nucleic acid delivery vectors with expression cassettes comprising the ubiquitous CBA promoter sequences disclosed herein were evaluated in a murine model of ultrasound mediated nucleic acid delivery to the liver.

### Experimental Animals and Protocol

Two groups of three RAG2 mice were evaluated as summarized in Table 11 below. Prior to the experiment, mice were implanted with a jugular vein catheter though which the sonoactive agent and the DNA solution was administered. The experimental groups were set forth below. In brief, Group 1 was administered a vector comprising a gene encoding a fluorescent reporter protein (FLUC) under a well-known CAG promoter which is known to have strong promoter activity in a variety of cell types, and Groups 2 was administered a vector comprising a fluorescent reporter protein (FLUC) coupled to a ubiquitous promoter of the present disclosure (SEQ ID NO: 37), with each group receiving administration of a sonoactive agent and ultrasound. Sonazoid was used as the ultrasound contrast agent in this experiment.

TABLE 11
Evaluation of Nucleic Acid Delivery Vectors in a Murine Model of Ultrasound Mediated Nucleic
Acid Transfection to the Liver Evaluating a Ubiquitous Promoter Sequence in the Liver

| | | DNA | | | | |
|---|---|---|---|---|---|---|
| Group | | Dose | Sonoactive | # of | | |
| (n = 3) | Strain | (ug) | agent | doses | Ultrasound | Vector |
| 1 | Rag2 | 250 | Sonazoid | 1 | C1-6, MI 1.4, 10 | SEQ ID NO: 42 (SEQ ID NO: 41 CAG |
| | | | | | sec on, 20 sec off, | promoter + FLUC reporter) |
| | | | | | 40 sec total on | |
| 2 | Rag2 | 250 | Sonazoid | 1 | C1-6, MI 1.4, 10 | SEQ ID NO: 43 (SEQ ID NO: 37 |
| | | | | | sec on, 20 sec off, | ubiquitous promoter + FLUC reporter) |
| | | | | | 40 sec total on | |

The mice received application of ultrasound energy following administration of 50 μL of the sonoactive agent and one third of the DNA payload in each treatment session. In a treatment session, ultrasound was applied using a C1-6 probe on a General Electric LOGIQ E9 system in Elasto mode applying an acoustic radiation force at a frequency of about 2.5 MHz at a mechanical index of 1.4 at a 100% push output at an ultrasound intensity of at least 200 mW/cm2 (ISPTA) for about 10 sec on, 20 sec off, for a total of 40 sec of ultrasound application over the liver region of each mouse.

Groups 1-2 mice were injected with D-Luciferin, potassium salt and imaged using IVIS 96 hours following the treatment session; and 2 weeks following the treatment session.

### Results

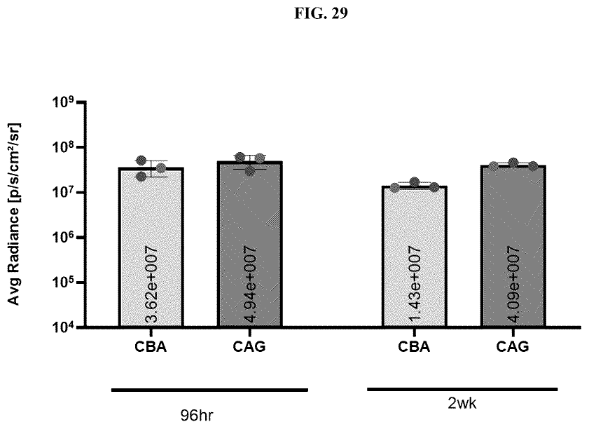

Results for Groups 1-2 are shown in FIG. 29, in which it is shown that Group 1 (CAG) mice exhibited average radiance levels of about 4.94 E7 p/s/cm2/sr at 96 hours following treatment, and exhibited average radiance levels of about 4.909 E7 p/s/cm2/sr at 2 weeks following treatment. Group 2 (CBA ubiquitous promoter sequence) exhibited average radiance levels of about 3.62 E7 p/s/cm2/sr at 96 hours following treatment, and exhibited average radiance levels of about 1.43 E7 p/s/cm2/sr at 2 weeks following treatment. The results of this experiment establish that the activity of the CBA promoter of SEQ ID NO 37 exhibits strong activity it the liver, comparable to that of the well-known CAG promoter.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

IV. SEQUENCE LISTING

| SEQ | | |
|---|---|---|
| ID NO | Description | SEQ |
| 1. | DMD-WT | ATGCTTTGGTGGGAAGAAGTAGAGGACTGTTATGAAAGAGAAGATGTTCAAAAGAAAACATTCAC |
| | | AAAATGGGTAAATGCACAATTTTCTAAGTTTGGGAAGCAGCATATTGAGAACCTCTTCAGTGACCTA |
| | | CAGGATGGGAGGCGCCTCCTAGACCTCCTCGAAGGCCTGACAGGGCAAAAACTGCCAAAAGAAA |
| | | AAGGATCCACAAGAGTTCATGCCCTGAACAATGTCAACAAGGCACTGCGGGTTTTGCAGAACAATA |
| | | ATGTTGATTTAGTGAATATTGGAAGTACTGACATCGTAGATGGAAATCATAAACTGACTCTTGGTTTG |
| | | ATTTGGAATATAATCCTCCACTGGCAGGTCAAAAATGTAATGAAAAATATCATGGCTGGATTGCAAC |
| | | AAACCAACAGTGAAAAGATTCTCCTGAGCTGGGTCCGACAATCAACTCGTAATTATCCACAGGTTA |
| | | ATGTAATCAACTTCACCACCAGCTGGTCTGATGGCCTGGCTTTGAATGCTCTCATCCATAGTCATAGG |
| | | CCAGACCTATTTGACTGGAATAGTGTGGTTTGCCAGCAGTCAGCCACACAACGACTGGAACATGCA |
| | | TTCAACATCGCCAGATATCAATTAGGCATAGAGAAACTACTCGATCCTGAAGATGTTGATACCACCT |
| | | ATCCAGATAAGAAGTCCATCTTAATGTACATCACATCACTCTTCCAAGTTTTGCCTCAACAAGTGAG |
| | | CATTGAAGCCATCCAGGAAGTGGAAATGTTGCCAAGGCCACCTAAAGTGACTAAAGAAGAACATTT |
| | | TCAGTTACATCATCAAATGCACTATTCTCAACAGATCACGGTCAGTCTAGCACAGGGATATGAGAGA |
| | | ACTTCTTCCCCTAAGCCTCGATTCAAGAGCTATGCCTACACACAGGCTGCTTATGTCACCACCTCTG |
| | | ACCCTACACGGAGCCCATTTCCTTCACAGCATTTGGAAGCTCCTGAAGACAAGTCATTTGGCAGTT |
| | | CATTGATGGAGAGTGAAGTAAACCTGGACCGTTATCAAACAGCTTTAGAAGAAGTATTATCGTGGCT |
| | | TCTTTCTGCTGAGGACACATTGCAAGCACAAGGAGAGATTTCTAATGATGTGGAAGTGGTGAAAGA |
| | | CCAGTTTCATACTCATGAGGGGTACATGATGGATTTGACAGCCCATCAGGGCCGGGTTGGTAATATT |
| | | CTACAATTGGGAAGTAAGCTGATTGGAACAGGAAAATTATCAGAAGATGAAGAAACTGAAGTACA |
| | | AGAGCAGATGAATCTCCTAAATTCAAGATGGGAATGCCTCAGGGTAGCTAGCATGGAAAAACAAAG |
| | | CAATTTACATAGAGTTTTAATGGATCTCCAGAATCAGAAACTGAAAGAGTTGAATGACTGGCTAACA |
| | | AAAACAGAAGAAAGAACAAGGAAAATGGAGGAAGAGCCTCTTGGACCTGATCTTGAAGACCTAA |
| | | AACGCCAAGTACAACAACATAAGGTGCTTCAAGAAGATCTAGAACAAGAACAAGTCAGGGTCAAT |
| | | TCTCTCACTCACATGGTGGTGGTAGTTGATGAATCTAGTGGAGATCACGCAACTGCTGCTTTGGAAG |
| | | AACAACTTAAGGTATTGGGAGATCGATGGGCAAACATCTGTAGATGGACAGAAGACCGCTGGGTTC |
| | | TTTTACAAGACATCCTTCTCAAATGGCAACGTCTTACTGAAGAACAGTGCCTTTTTAGTGCATGGCT |
| | | TTCAGAAAAAGAAGATGCAGTGAACAAGATTCACACAACTGGCTTTAAAGATCAAAATGAAATGTT |
| | | ATCAAGTCTTCAAAAACTGGCCGTTTTAAAAGCGGATCTAGAAAAGAAAAAGCAATCCATGGGCA |
| | | AACTGTATTCACTCAAACAAGATCTTCTTTCAACACTGAAGAATAAGTCAGTGACCCAGAAGACGG |
| | | AAGCATGGCTGGATAACTTTGCCCGGTGTTGGGATAATTTAGTCCAAAAACTTGAAAAGAGTACAG |
| | | CACAGATTTCACAGGCTGTCACCACCACTCAGCCATCACTAACACAGACAACTGTAATGGAAACAG |
| | | TAACTACGGTGACCACAAGGGAACAGATCCTGGTAAAGCATGCTCAAGAGGAACTTCCACCACCA |
| | | CCTCCCCAAAAGAAGAGGCAGATTACTGTGGATTCTGAAATTAGGAAAAGGTTGGATGTTGATATA |
| | | ACTGAACTTCACAGCTGGATTACTCGCTCAGAAGCTGTGTTGCAGAGTCCTGAATTTGCAATCTTTC |
| | | GGAAGGAAGGCAACTTCTCAGACTTAAAAGAAAAAGTCAATGCCATAGAGCGAGAAAAAGCTGA |
| | | GAAGTTCAGAAAACTGCAAGATGCCAGCAGATCAGCTCAGGCCCTGGTGGAACAGATGGTGAATG |
| | | AGGGTGTTAATGCAGATAGCATCAAACAAGCCTCAGAACAACTGAACAGCCGGTGGATCGAATTCT |
| | | GCCAGTTGCTAAGTGAGAGACTTAACTGGCTGGAGTATCAGAACAACATCATCGCTTTCTATAATCA |
| | | GCTACAACAATTGGAGCAGATGACAACTACTGCTGAAAACTGGTTGAAAATCCAACCCACCACCCC |
| | | ATCAGAGCCAACAGCAATTAAAAGTCAGTTAAAAATTTGTAAGGATGAAGTCAACCGGCTATCAGA |
| | | TCTTCAACCTCAAATTGAACGATTAAAAATTCAAAGCATAGCCCTGAAAGAGAAAGGACAAGGAC |
| | | CCATGTTCCTGGATGCAGACTTTGTGGCCTTTACAAATCATTTTAAGCAAGTCTTTTCTGATGTGCAG |
| | | GCCAGAGAGAAAGAGCTACAGACAATTTTTGACACTTTGCCACCAATGCGCTATCAGGAAACCATG |
| | | AGTGCCATCAGGACATGGGTCCAGCAGTCAGAAACCAAACTCTCCATACCTCAACTTAGTGTCACC |
| | | GACTATGAAATCATGGAGCAGAGACTCGGGGAATTGCAGGCTTTACAAAGTTCTCTGCAAGAGCAA |
| | | CAAAGTGGCCTATACTATCTCAGCACCACTGTGAAAGAGATGTCGAAGAAAGCGCCCTCTGAAATT |
| | | AGCCGGAAATATCAATCAGAATTTGAAGAAATTGAGGGACGCTGGAAGAAGCTCTCCTCCCAGCTG |
| | | GTTGAGCATTGTCAAAAGCTAGAGGAGCAAATGAATAAACTCCGAAAAATTCAGAATCACATACAA |
| | | ACCCTGAAGAAATGGATGGCTGAAGTTGATGTTTTTCTGAAGGAGGAATGGCCTGCCCTTGGGGAT |
| | | TCAGAAATTCTAAAAAAGCAGCTGAAACAGTGCAGACTTTTAGTCAGTGATATTCAGACAATTCAG |
| | | CCCAGTCTAAACAGTGTCAATGAAGGTGGGCAGAAGATAAAGAATGAAGCAGAGCCAGAGTTTGC |
| | | TTCGAGACTTGAGACAGAACTCAAAGAACTTAACACTCAGTGGGATCACATGTGCCAACAGGTCTA |
| | | TGCCAGAAAGGAGGCCTTGAAGGGAGGTTTGGAGAAAACTGTAAGCCTCCAGAAAGATCTATCAG |
| | | AGATGCACGAATGGATGACACAAGCTGAAGAAGAGTATCTTGAGAGAGATTTTGAATATAAAACTC |
| | | CAGATGAATTACAGAAAGCAGTTGAAGAGATGAAGAGAGCTAAAGAAGAGGCCCAACAAAAAGA |
| | | AGCGAAAGTGAAACTCCTTACTGAGTCTGTAAATAGTGTCATAGCTCAAGCTCCACCTGTAGCACA |
| | | AGAGGCCTTAAAAAAGGAACTTGAAACTCTAACCACCAACTACCAGTGGCTCTGCACTAGGCTGA |
| | | ATGGGAAATGCAAGACTTTGGAAGAAGTTTGGGCATGTTGGCATGAGTTATTGTCATACTTGGAGA |
| | | AAGCAAACAAGTGGCTAAATGAAGTAGAATTTAAACTTAAAACCACTGAAAACATTCCTGGCGGA |
| | | GCTGAGGAAATCTCTGAGGTGCTAGATTCACTTGAAAATTTGATGCGACATTCAGAGGATAACCCA |
| | | AATCAGATTCGCATATTGGCACAGACCCTAACAGATGGCGGAGTCATGGATGAGCTAATCAATGAGG |
| | | AACTTGAGACATTTAATTCTCGTTGGAGGGAACTACATGAAGAGGCTGTAAGGAGGCAAAAGTTGC |
| | | TTGAACAGAGCATCCAGTCTGCCCAGGAGACTGAAAAATCCTTACACTTAATCCAGGAGTCCCTCA |
| | | CATTCATTGACAAGCAGTTGGCAGCTTATATTGCAGACAAGGTGGACGCAGCTCAAATGCCTCAGG |
| | | AAGCCCAGAAAATCCAATCTGATTTGACAAGTCATGAGATCAGTTTAGAAGAAATGAAGAAACATA |
| | | ATCAGGGGAAGGAGGCTGCCCAAAGAGTCCTGTCTCAGATTGATGTTGCACAGAAAAAATTACAA |
| | | GATGTCTCCATGAAGTTTCGATTATTCCAGAAACCAGCCAATTTTGAGCAGCGTCTACAAGAAAGTA |
| | | AGATGATTTTAGATGAAGTGAAGATGCACTTGCCTGCATTGGAAACAAAGAGTGTGGAACAGGAA |
| | | GTAGTACAGTCACAGCTAAATCATTGTGTGAACTTGTATAAAAGTCTGAGTGAAGTGAAGTCTGAA |
| | | GTGGAAATGGTGATAAAGACTGGACGTCAGATTGTACAGAAAAAGCAGACGGAAAATCCCAAAGA |
| | | ACTTGATGAAAGAGTAACAGCTTTGAAATTGCATTATAATGAGCTGGGAGCAAAGGTAACAGAAAG |
| | | AAAGCAACAGTTGGAGAAATGCTTGAAATTGTCCCGTAAGATGCGAAAGGAAATGAATGTCTTGAC |
| | | AGAATGGCTGGCAGCTACAGATATGGAATTGACAAAGAGATCAGCAGTTGAAGGAATGCCTAGTAA |
| | | TTTGGATTCTGAAGTTGCCTGGGGAAAGGCTACTCAAAAAGAGATTGAGAAACAGAAGGTGCACC |
| | | TGAAGAGTATCACAGAGGTAGGAGAGGCCTTGAAAACAGTTTTGGGCAAGAAGGAGACGTTGGTG |
| | | GAAGATAAACTCAGTCTTCTGAATAGTAACTGGATAGCTGTCACCTCCCGAGCAGAAGAGTGGTTA |
| | | AATCTTTTGTTGGAATACCAGAAACACATGGAAACTTTTGACCAGAATGTGGACCACATCACAAAG |
| | | TGGATCATTCAGGCTGACACACTTTTGGATGAATCAGAGAAAAAGAAACCCCAGCAAAAAGAAGA |
| | | CGTGCTTAAGCGTTTAAAGGCAGAACTGAATGACATACGCCCAAAGGTGGACTCTACACGTGACCA |
| | | AGCAGCAAACTTGATGGCAAACCGCGGTGACCACTGCAGGAAATTAGTAGAGCCCCAAATCTCAG |
| | | AGCTCAACCATCGATTTGCAGCCATTTCACACAGAATTAAGACTGGAAAGGCCTCCATTCCTTTGAA |
| | | GGAATTGGAGCAGTTTAACTCAGATATACAAAAATTGCTTGAACCACTGGAGGCTGAAATTCAGCA |
| | | GGGGGTGAATCTGAAAGAGGAAGACTTCAATAAAGATATGAATGAAGACAATGAGGGTACTGTAA |
| | | AAGAATTGTTGCAAAGAGGAGACAACTTACAACAAAGAATCACAGATGAGAGAAAGCGAGAGGA |
| | | AATAAAGATAAAACAGCAGCTGTTACAGACAAAACATAATGCTCTCAAGGATTTGAGGAGCCAAAG |
| | | AAGAAAAAAGGCTCTAGAAATTTCTCATCAGTGGTATCAGTACAAGAGGCAGGCTGATGATCTCCT |
| | | GAAATGCTTGGATGACATTGAAAAAAAATTAGCCAGCCTACCTGAGCCCAGAGATGAAAGGAAAAT |
| | | AAAGGAAATTGATCGGGAATTGCAGAAGAAGAAAGAGGAGCTGAATGCAGTGCGTAGGCAAGCTG |
| | | AGGGCTTGTCTGAGGATGGGGCCGCAATGGCAGTGGAGCCAACTCAGATCCAGCTCAGCAAGCGC |
| | | TGGCGGGAAATTGAGAGCAAATTTGCTCAGTTTCGAAGACTCAACTTTGCACAAATTCACACTGTC |
| | | CGTGAAGAAACGATGATGGTGATGACTGAAGACATGCCTTTGGAAATTTCTTATGTGCCTTCTACTT |
| | | ATTTGACTGAAATCACTCATGTCTCACAAGCCCTATTAGAAGTGGAACAACTTCTCAATGCTCCTGA |
| | | CCTCTGTGCTAAGGACTTTGAAGATCTCTTTAAGCAAGAGGAGTCTCTGAAGAATATAAAAGATAGT |
| | | CTACAACAAAGCTCAGGTCGGATTGACATTATTCATAGCAAGAAGACAGCAGCATTGCAAAGTGCA |
| | | ACGCCTGTGGAAAGGGTGAAGCTACAGGAAGCTCTCTCCCAGCTTGATTTCCAATGGGAAAAAGTT |
| | | AACAAAATGTACAAGGACCGACAAGGGCGATTTGACAGATCTGTTGAGAAATGGCGGCGTTTTCAT |
| | | TATGATATAAAGATATTTAATCAGTGGCTAACAGAAGCTGAACAGTTTCTCAGAAAGACACAAATTC |
| | | CTGAGAATTGGGAACATGCTAAATACAAATGGTATCTTAAGGAACTCCAGGATGGCATTGGGCAGC |
| | | GGCAAACTGTTGTCAGAACATTGAATGCAACTGGGGAAGAAATAATTCAGCAATCCTCAAAAACAG |
| | | ATGCCAGTATTCTACAGGAAAAATTGGGAAGCCTGAATCTGCGGTGGCAGGAGGTCTGCAAACAGC |
| | | TGTCAGACAGAAAAAAGAGGCTAGAAGAACAAAAGAATATCTTGTCAGAATTTCAAAGAGATTTA |
| | | AATGAATTTGTTTTATGGTTGGAGGAAGCAGATAACATTGCTAGTATCCCACTTGAACCTGGAAAAG |
| | | AGCAGCAACTAAAAGAAAAGCTTGAGCAAGTCAAGTTACTGGTGGAAGAGTTGCCCCTGCGCCAG |
| | | GGAATTCTCAAACAATTAAATGAAACTGGAGGACCCGTGCTTGTAAGTGCTCCCATAAGCCCAGAA |
| | | GAGCAAGATAAACTTGAAAATAAGCTCAAGCAGACAAATCTCCAGTGGATAAAGGTTTCCAGAGCT |
| | | TTACCTGAGAAACAAGGAGAAATTGAAGCTCAAATAAAAGACCTTGGGCAGCTTGAAAAAAAGCT |
| | | TGAAGACCTTGAAGAGCAGTTAAATCATCTGCTGCTGTGGTTATCTCCTATTAGGAATCAGTTGGAA |
| | | ATTTATAACCAACCAAACCAAGAAGGACCATTTGACGTTAAGGAAACTGAAATAGCAGTTCAAGCT |
| | | AAACAACCGGATGTGGAAGAGATTTTGTCTAAAGGGCAGCATTTGTACAAGGAAAAACCAGCCAC |
| | | TCAGCCAGTGAAGAGGAAGTTAGAAGATCTGAGCTCTGAGTGGAAGGCGGTAAACCGTTTACTTC |
| | | AAGAGCTGAGGGCAAAGCAGCCTGACCTAGCTCCTGGACTGACCACTATTGGAGCCTCTCCTACTC |
| | | AGACTGTTACTCTGGTGACACAACCTGTGGTTACTAAGGAAACTGCCATCTCCAAACTAGAAATGC |
| | | CATCTTCCTTGATGTTGGAGGTACCTGCTCTGGCAGATTTCAACCGGGCTTGGACAGAACTTACCGA |
| | | CTGGCTTTCTCTGCTTGATCAAGTTATAAAATCACAGAGGGTGATGGTGGGTGACCTTGAGGATATC |
| | | AACGAGATGATCATCAAGCAGAAGGCAACAATGCAGGATTTGGAACAGAGGCGTCCCCAGTTGGA |
| | | AGAACTCATTACCGCTGCCCAAAATTTGAAAAACAAGACCAGCAATCAAGAGGCTAGAACAATCAT |
| | | TACGGATCGAATTGAAAGAATTCAGAATCAGTGGGATGAAGTACAAGAACACCTTCAGAACCGGA |
| | | GGCAACAGTTGAATGAAATGTTAAAGGATTCAACACAATGGCTGGAAGCTAAGGAAGAAGCTGAG |
| | | CAGGTCTTAGGACAGGCCAGAGCCAAGCTTGAGTCATGGAAGGAGGGTCCCTATACAGTAGATGCA |
| | | ATCCAAAAGAAAATCACAGAAACCAAGCAGTTGGCCAAAGACCTCCGCCAGTGGCAGACAAATGT |
| | | AGATGTGGCAAATGACTTGGCCCTGAAACTTCTCCGGGATTATTCTGCAGATGATACCAGAAAAGTC |
| | | CACATGATAACAGAGAATATCAATGCCTCTTGGAGAAGCATTCATAAAAGGGTGAGTGAGCGAGAG |
| | | GCTGCTTTGGAAGAAACTCATAGATTACTGCAACAGTTCCCCCTGGACCTGGAAAAGTTTCTTGCC |
| | | TGGCTTACAGAAGCTGAAACAACTGCCAATGTCCTACAGGATGCTACCCGTAAGGAAAGGCTCCTA |
| | | GAAGACTCCAAGGGAGTAAAAGAGCTGATGAAACAATGGCAAGACCTCCAAGGTGAAATTGAAGC |
| | | TCACACAGATGTTTATCACAACCTGGATGAAAACAGCCAAAAAATCCTGAGATCCCTGGAAGGTTC |
| | | CGATGATGCAGTCCTGTTACAAAGACGTTTGGATAACATGAACTTCAAGTGGAGTGAACTTCGGAA |
| | | AAAGTCTCTCAACATTAGGTCCCATTTGGAAGCCAGTTCTGACCAGTGGAAGCGTCTGCACCTTTC |
| | | TCTGCAGGAACTTCTGGTGTGGCTACAGCTGAAAGATGATGAATTAAGCCGGCAGGCACCTATTGG |
| | | AGGCGACTTTCCAGCAGTTCAGAAGCAGAACGATGTACATAGGGCCTTCAAGAGGGAATTGAAAA |
| | | CTAAAGAACCTGTAATCATGAGTACTCTTGAGACTGTACGAATATTTCTGACAGAGCAGCCTTTGGA |
| | | AGGACTAGAGAAACTCTACCAGGAGCCCAGAGAGCTGCCTCCTGAGGAGAGAGCCCAGAATGTCA |
| | | CTCGGCTTCTACGAAAGCAGGCTGAGGAGGTCAATACTGAGTGGGAAAAATTGAACCTGCACTCC |
| | | GCTGACTGGCAGAGAAAAATAGATGAAACCCTTGAAAGACTCCGGGAACTTCAAGAGGCCACGGA |
| | | TGAGCTGGACCTCAAGCTGCGCCAAGCTGAGGTGATCAAGGGATCCTGGCAGCCCGTGGGCGATC |
| | | TCCTCATTGACTCTCTCCAAGATCACCTGGAGAAAGTCAAGGCACTTCGAGGAGAAATTGCGCCTC |
| | | TGAAAGAGAACGTGAGCCACGTCAATGACCTTGCTCGCCAGCTTACCACTTTGGGCATTCAGCTCT |
| | | CACCGTATAACCTCAGCACTCTGGAAGACCTGAACACCAGATGGAAGCTTCTGCAGGTGGCCGTCG |
| | | AGGACCGAGTCAGGCAGCTGCATGAAGCCCACAGGGACTTTGGTCCAGCATCTCAGCACTTTCTTT |
| | | CCACGTCTGTCCAGGGTCCCTGGGAGAGAGCCATCTCGCCAAACAAAGTGCCCTACTATATCAACC |
| | | ACGAGACTCAAACAACTTGCTGGGACCATCCCAAAATGACAGAGCTCTACCAGTCTTTAGCTGACC |
| | | TGAATAATGTCAGATTCTCAGCTTATAGGACTGCCATGAAACTCCGAAGACTGCAGAAGGCCCTTTG |
| | | CTTGGATCTCTTGAGCCTGTCAGCTGCATGTGATGCCTTGGACCAGCACAACCTCAAGCAAAATGA |
| | | CCAGCCCATGGATATCCTGCAGATTATTAATTGTTTGACCACTATTTATGACCGCCTGGAGCAAGAGC |
| | | ACAACAATTTGGTCAACGTCCCTCTCTGCGTGGATATGTGTCTGAACTGGCTGCTGAATGTTTATGA |
| | | TACGGGACGAACAGGGAGGATCCGTGTCCTGTCTTTTAAAACTGGCATCATTTCCCTGTGTAAAGC |
| | | ACATTTGGAAGACAAGTACAGATACCTTTTCAAGCAAGTGGCAAGTTCAACAGGATTTTGTGACCA |
| | | GCGCAGGCTGGGCCTCCTTCTGCATGATTCTATCCAAATTCCAAGACAGTTGGGTGAAGTTGCATCC |
| | | TTTGGGGGCAGTAACATTGAGCCAAGTGTCCGGAGCTGCTTCCAATTTGCTAATAATAAGCCAGAG |
| | | ATCGAAGCGGCCCTCTTCCTAGACTGGATGAGACTGGAACCCCAGTCCATGGTGTGGCTGCCCGTC |
| | | CTGCACAGAGTGGCTGCTGCAGAAACTGCCAAGCATCAGGCCAAATGTAACATCTGCAAAGAGTG |
| | | TCCAATCATTGGATTCAGGTACAGGAGTCTAAAGCACTTTAATTATGACATCTGCCAAAGCTGCTTTT |
| | | TTTCTGGTCGAGTTGCAAAAGGCCATAAAATGCACTATCCCATGGTGGAATATTGCACTCCGACTAC |
| | | ATCAGGAGAAGATGTTCGAGACTTTGCCAAGGTACTAAAAAACAAATTTCGAACCAAAAGGTATTT |
| | | TGCGAAGCATCCCCGAATGGGCTACCTGCCAGTGCAGACTGTCTTAGAGGGGGACAACATGGAAA |
| | | CTCCCGTTACTCTGATCAACTTCTGGCCAGTAGATTCTGCGCCTGCCTCGTCCCCTCAGCTTTCACA |
| | | CGATGATACTCATTCACGCATTGAACATTATGCTAGCAGGCTAGCAGAAATGGAAAACAGCAATGGA |
| | | TCTTATCTAAATGATAGCATCTCTCCTAATGAGAGCATAGATGATGAACATTTGTTAATCCAGCATTAC |
| | | TGCCAAAGTTTGAACCAGGACTCCCCCCTGAGCCAGCCTCGTAGTCCTGCCCAGATCTTGATTTCCT |
| | | TAGAGAGTGAGGAAAGAGGGGAGCTAGAGAGAATCCTAGCAGATCTTGAGGAAGAAAACAGGAA |
| | | TCTGCAAGCAGAATATGACCGTCTAAAGCAGCAGCACGAACATAAAGGCCTGTCCCCACTGCCGTC |
| | | CCCTCCTGAAATGATGCCCACCTCTCCCCAGAGTCCCCGGGATGCTGAGCTCATTGCTGAGGCCAA |
| | | GCTACTGCGTCAACACAAAGGCCGCCTGGAAGCCAGGATGCAAATCCTGGAAGACCACAATAAAC |
| | | AGCTGGAGTCACAGTTACACAGGCTAAGGCAGCTGCTGGAGCAACCCCAGGCAGAGGCCAAAGT |
| | | GAATGGCACAACGGTGTCCTCTCCTTCTACCTCTCTACAGAGGTCCGACAGCAGTCAGCCTATGCTG |
| | | CTCCGAGTGGTTGGCAGTCAAACTTCGGACTCCATGGGTGAGGAAGATCTTCTCAGTCCTCCCCAG |
| | | GACACAAGCACAGGGTTAGAGGAGGTGATGGAGCAACTCAACAACTCCTTCCCTAGTTCAAGAGG |
| | | AAGAAATACCCCTGGAAAGCCAATGAGAGAGGACACAATGTAGTAA |
| 2. | DMD-3 | ATGCTGTGGTGGGAAGAGGTGGAGGACTGTTACGAGAGAGAGGACGTGCAGAAGAAGACATTCA |
| | | CCAAGTGGGTGAACGCTCAGTTCAGCAAGTTCGGCAAGCAGCACATCGAGAACCTGTTTAGCGAT |
| | | CTACAAGATGGCAGAAGACTGCTGGACCTGCTGGAGGGCCTGACCGGGCAGAAGCTGCCCAAAGA |
| | | AAAGGGGTCTACAAGAGTGCACGCCCTGAACAACGTGAACAAGGCCCTGAGAGTGCTGCAGAAC |
| | | AACAACGTGGACCTGGTGAACATCGGCAGCACCGACATCGTGGACGGCAACCACAAGCTGACCCT |
| | | GGGCCTGATCTGGAACATCATCCTGCACTGGCAAGTGAAGAACGTGATGAAGAACATCATGGCCGG |
| | | CCTGCAGCAGACCAACAGCGAGAAGATCCTGCTGAGCTGGGTGAGACAGAGCACAAGAAACTAC |
| | | CCCCAAGTGAACGTGATCAACTTCACCACAAGCTGGAGCGACGGCCTGGCCCTGAACGCCCTGAT |
| | | CCACAGCCACAGACCCGACCTGTTCGACTGGAACAGCGTGGTGTGTCAGCAGAGCGCCACACAGA |
| | | GACTGGAGCACGCCTTCAACATCGCTAGATATCAGCTGGGCATCGAGAAGTTACTGGACCCCGAAG |
| | | ATGTGGACACCACCTACCCCGACAAGAAGAGCATCCTGATGTACATCACAAGCCTGTTCCAAGTGC |
| | | TGCCTCAGCAAGTGAGCATCGAGGCCATCCAAGAAGTCGAGATGCTGCCTAGACCCCCCAAGGTG |
| | | ACCAAGGAGGAGCACTTTCAGCTGCACCATCAGATGCACTACTCTCAGCAGATCACAGTCAGCTTG |
| | | GCACAAGGCTACGAGAGAACAAGCAGCCCCAAGCCTAGATTCAAGAGCTACGCCTACACCCAAGC |
| | | CGCCTACGTGACCACAAGTGACCCCACAAGAAGCCCCTTCCCTTCTCAGCACCTGGAGGCCCCCG |
| | | AGGACAAGAGCTTCGGCAGCAGCCTGATGGAGAGCGAGGTGAACCTGGACAGATATCAGACCGCG |
| | | TTAGAAGAAGTGTTGAGCTGGCTGCTGAGCGCCGAGGACACCCTGCAAGCCCAAGGCGAGATCAG |
| | | CAACGATGTGGAGGTGGTGAAGGATCAGTTCCACACCCACGAGGGCTACATGATGGACCTGACCG |
| | | CCCACCAAGGCAGAGTGGGCAACATCCTGCAGCTGGGCAGCAAGCTGATCGGCACCGGCAAGCTG |
| | | AGCGAGGACGAGGAAACCGAGGTTCAAGAGCAAATGAACCTGCTAAACAGCAGATGGGAGTGCC |
| | | TGAGAGTGGCTAGCATGGAAAAGCAGAGTAACCTGCACAGAGTGCTGATGGACCTGCAGAATCAG |
| | | AAATTGAAAGAACTTAATGATTGGCTGACCAAGACCGAGGAGAGAACAAGAAAGATGGAGGAGG |
| | | AGCCCCTGGGCCCCGACCTGGAGGACCTGAAGAGACAAGTGCAGCAGCACAAGGTGCTGCAAGA |
| | | GGACCTGGAGCAAGAGCAAGTGAGAGTGAACTCTCTGACACACATGGTGGTGGTAGTGGACGAGA |
| | | GCAGCGGCGATCACGCTACCGCGGCGCTGGAAGAGCAGCTGAAAGTGCTGGGCGACAGATGGGCC |
| | | AACATCTGCAGATGGACCGAGGACAGATGGGTGCTGCTGCAAGACATCCTGCTGAAGTGGCAGAG |
| | | ACTGACCGAGGAGCAGTGCCTGTTCAGCGCCTGGCTGAGCGAGAAGGAAGACGCTGTGAACAAG |
| | | ATCCACACCACCGGCTTCAAGGATCAGAACGAGATGCTGAGCTCCCTCCAAAAGCTCGCAGTGCTG |
| | | AAGGCCGACCTGGAAAAGAAAAAACAGTCGATGGGCAAGCTGTACAGCCTGAAGCAAGACCTGC |
| | | TGAGCACCCTGAAGAACAAGAGCGTGACACAGAAGACCGAGGCCTGGCTGGACAACTTCGCTAG |
| | | ATGCTGGGACAACCTGGTACAGAAGTTAGAGAAGTCTACCGCTCAGATCAGCCAAGCCGTAACCAC |
| | | CACACAACCTAGCCTGACTCAGACCACCGTGATGGAAACCGTGACCACCGTGACAACCCGTGAGC |
| | | AGATCCTGGTGAAGCACGCCCAAGAGGAGCTGCCCCCCCCCCCCCCTCAGAAGAAGAGACAGATC |
| | | ACCGTGGACTCAGAGATTCGCAAGAGACTGGACGTGGACATCACCGAGCTGCACAGCTGGATCAC |
| | | AAGAAGCGAGGCCGTGCTGCAGAGCCCTGAGTTCGCGATCTTTAGAAAGGAGGGCAACTTCAGCG |
| | | ACTTGAAGGAGAAAGTGAACGCCATCGAGAGAGAGAAGGCCGAGAAGTTCAGAAAGTTGCAAGA |
| | | CGCCTCAAGAAGCGCCCAAGCCCTGGTGGAGCAGATGGTTAACGAAGGCGTGAATGCCGACAGCA |
| | | TCAAGCAAGCTAGCGAGCAGCTGAATAGCCGGTGGATTGAATTCTGTCAGCTGCTGAGCGAGAGAT |
| | | TAAACTGGCTGGAGTATCAGAACAACATCATCGCCTTCTACAATCAGCTGCAGCAACTAGAACAAA |
| | | TGACCACCACCGCCGAGAACTGGCTAAAGATTCAGCCCACCACCCCTAGCGAGCCCACCGCCATCA |
| | | AATCTCAGCTGAAAATTTGCAAGGACGAAGTGAACAGACTGTCGGACCTGCAGCCTCAAATCGAA |
| | | AGACTGAAAATTCAGAGCATCGCACTTAAGGAGAAGGGCCAAGGACCCATGTTCCTGGACGCCGA |
| | | CTTCGTGGCCTTCACCAACCACTTCAAGCAAGTGTTCAGCGACGTGCAAGCTAGAGAGAAGGAGC |
| | | TGCAGACCATCTTCGACACCCTGCCCCCCATGAGATACCAAGAAACCATGAGCGCCATCAGAACCT |
| | | GGGTGCAGCAGAGCGAAACCAAGCTGAGCATCCCTCAGCTGAGCGTGACCGACTACGAGATCATG |
| | | GAGCAAAGACTGGGAGAGCTGCAAGCCCTGCAGAGCAGCCTGCAAGAGCAGCAGAGCGGCCTGT |
| | | ACTACCTGAGCACCACCGTGAAGGAGATGTCGAAGAAAGCCCCGTCGGAGATCAGCAGAAAGTAT |
| | | CAGAGCGAGTTCGAGGAGATCGAGGGCAGATGGAAGAAGCTGAGCAGCCAACTGGTGGAACACT |
| | | GTCAGAAATTAGAAGAACAGATGAACAAGCTGAGAAAGATTCAGAACCACATTCAGACCCTGAAG |
| | | AAGTGGATGGCCGAGGTGGACGTGTTCCTGAAAGAGGAGTGGCCCGCCCTGGGCGATTCCGAAAT |
| | | CTTAAAAAAGCAGCTGAAGCAGTGCAGACTGCTGGTGTCAGATATCCAAACCATTCAACCAAGCCT |
| | | GAACAGCGTGAACGAAGGCGGGCAGAAGATCAAGAACGAGGCCGAGCCCGAGTTCGCTAGCAGA |
| | | CTGGAAACCGAGCTGAAGGAGCTGAATACACAGTGGGACCACATGTGTCAGCAAGTGTACGCTAG |
| | | AAAGGAAGCGCTGAAGGGCGGGCTGGAGAAGACCGTTAGCCTGCAAAAGGATCTGAGCGAGATG |
| | | CACGAGTGGATGACCCAAGCCGAGGAGGAGTACCTGGAGAGAGACTTCGAGTACAAGACCCCCG |
| | | ACGAGCTGCAGAAGGCCGTCGAGGAAATGAAGAGAGCGAAGGAGGAGGCTCAGCAGAAGGAAG |
| | | CCAAGGTGAAGCTGCTGACTGAATCTGTCAACAGTGTGATAGCTCAAGCACCCCCCGTTGCCCAAG |
| | | AGGCACTAAAGAAGGAGCTGGAAACTCTGACCACCAACTATCAGTGGCTGTGCACAAGACTGAAC |
| | | GGCAAGTGCAAGACCCTGGAGGAAGTGTGGGCCTGCTGGCACGAGCTGCTGAGCTACCTGGAGAA |
| | | GGCCAACAAGTGGCTGAACGAGGTGGAGTTCAAGCTGAAGACCACCGAGAACATCCCCGGCGGC |
| | | GCCGAGGAGATCAGCGAGGTGCTGGACAGCCTGGAGAACCTGATGAGACACAGCGAGGACAACC |
| | | CCAATCAGATCAGAATCCTGGCTCAGACCCTGACCGACGGCGGCGTGATGGACGAGCTGATCAACG |
| | | AGGAGCTGGAAACCTTTAACTCTAGATGGCGAGAACTGCACGAGGAGGCCGTGAGAAGACAGAA |
| | | GCTGCTGGAGCAAAGCATTCAGAGCGCCCAAGAAACCGAGAAGAGCCTGCACCTGATCCAAGAG |
| | | AGCCTGACCTTCATCGACAAGCAGCTGGCCGCCTACATCGCCGACAAGGTGGACGCCGCTCAGATG |
| | | CCACAAGAGGCTCAGAAGATTCAGTCCGATCTGACAAGCCACGAGATCAGCCTGGAGGAGATGAA |
| | | AAAACACAACCAAGGCAAGGAGGCCGCTCAGAGAGTGCTGTCTCAGATCGACGTGGCTCAGAAG |
| | | AAGCTGCAAGACGTGAGCATGAAGTTCAGACTGTTTCAGAAGCCCGCCAACTTCGAGCAGCGACT |
| | | GCAAGAGAGCAAGATGATCCTGGACGAGGTGAAGATGCACCTGCCCGCCCTGGAAACCAAGAGCG |
| | | TGGAGCAAGAGGTGGTGCAGTCTCAGTTGAACCACTGCGTGAACCTGTACAAGAGCCTGTCGGAG |
| | | GTCAAGAGTGAGGTCGAGATGGTGATCAAAACTGGGAGACAGATCGTGCAAAAAAAGCAGACCG |
| | | AGAACCCCAAGGAGCTGGACGAGCGTGTTACCGCCCTGAAGCTGCACTACAACGAGCTGGGCGCC |
| | | AAGGTGACCGAGAGAAAGCAGCAGCTGGAGAAGTGCCTGAAGCTGAGCAGAAAGATGAGAAAG |
| | | GAGATGAACGTGCTGACCGAGTGGCTGGCCGCCACCGACATGGAGCTGACCAAGAGAAGCGCCGT |
| | | GGAGGGCATGCCTAGCAACCTGGACAGCGAGGTGGCCTGGGGCAAGGCCACACAGAAGGAGATC |
| | | GAGAAGCAGAAGGTGCACCTGAAGAGCATCACCGAGGTGGGCGAGGCCCTGAAGACCGTGCTGG |
| | | GCAAGAAGGAAACCCTGGTGGAGGACAAGCTGAGCCTGCTGAACTCGAACTGGATCGCCGTGAC |
| | | AAGCAGAGCCGAGGAGTGGCTGAACCTGCTGTTAGAGTACCAAAAACACATGGAAACCTTCGATC |
| | | AGAACGTGGACCACATCACCAAGTGGATCATCCAAGCCGACACCCTGCTGGACGAAAGCGAAAAG |
| | | AAGAAGCCGCAACAAAAGGAGGATGTGCTGAAGAGACTGAAGGCCGAACTGAACGATATCAGAC |
| | | CCAAGGTGGACAGCACACGGGACCAAGCCGCCAACCTGATGGCCAACAGAGGCGACCACTGCAG |
| | | AAAACTGGTGGAGCCTCAGATCAGCGAGCTGAACCACAGATTCGCCGCCATCAGCCACAGAATCA |
| | | AAACCGGCAAAGCGAGCATCCCCTTGAAGGAACTGGAGCAATTTAACAGCGACATCCAAAAATTG |
| | | CTCGAACCACTGGAGGCCGAGATTCAGCAAGGCGTGAACCTGAAGGAAGAGGACTTCAACAAGG |
| | | ACATGAACGAGGACAACGAGGGCACCGTGAAAGAACTGCTGCAGAGAGGCGACAACCTGCAGCA |
| | | GAGAATCACCGACGAGAGAAAGAGAGAGGAGATCAAGATCAAGCAGCAGCTGCTGCAGACTAAG |
| | | CACAACGCCCTGAAGGATCTGAGATCTCAGAGGAGAAAAAAGGCTCTGGAAATTTCACATCAGTG |
| | | GTATCAGTACAAGAGACAAGCAGACGACCTGCTGAAGTGCCTGGACGACATCGAGAAGAAGTTAG |
| | | CTAGCCTGCCCGAGCCTAGAGACGAGAGAAAGATCAAGGAGATCGACAGAGAGTTACAAAAGAA |
| | | GAAGGAGGAGCTGAATGCGGTAAGAAGACAAGCGGAAGGCCTGTCCGAGGACGGCGCAGCCATG |
| | | GCCGTGGAGCCCACACAGATTCAGCTCAGCAAGAGATGGAGAGAGATCGAGAGCAAGTTCGCTCA |
| | | GTTCAGAAGACTGAACTTCGCTCAGATCCACACCGTGAGAGAGGAAACCATGATGGTGATGACCG |
| | | AGGACATGCCCCTGGAGATCTCATACGTTCCTAGCACCTACCTGACCGAGATCACCCACGTGAGCC |
| | | AAGCCCTGCTGGAGGTGGAGCAGCTGCTGAACGCCCCCGACCTGTGCGCCAAGGACTTCGAGGAC |
| | | CTGTTCAAACAAGAGGAGAGCCTGAAGAACATCAAGGACAGCCTGCAGCAAAGTAGCGGCAGAA |
| | | TCGACATCATCCACAGCAAGAAGACCGCCGCCCTGCAGAGCGCCACCCCCGTGGAGAGAGTGAAG |
| | | CTTCAAGAGGCCCTTTCGCAGCTGGATTTTCAGTGGGAGAAGGTGAACAAAATGTACAAGGACAG |
| | | ACAAGGCAGATTCGACAGAAGCGTGGAGAAGTGGAGAAGATTCCACTACGACATCAAGATCTTCA |
| | | ATCAGTGGCTGACCGAGGCAGAGCAGTTCCTGAGAAAGACACAGATCCCCGAGAACTGGGAGCAC |
| | | GCCAAGTACAAGTGGTACCTGAAGGAGCTGCAAGACGGCATCGGGCAGAGACAGACCGTGGTGA |
| | | GAACCCTGAACGCCACCGGCGAGGAGATCATCCAACAAAGCTCCAAAACCGACGCTAGCATCCTG |
| | | CAAGAGAAACTGGGCAGCCTGAACCTGAGATGGCAAGAGGTGTGCAAGCAGCTGAGCGACCGCA |
| | | AAAAACGGTTAGAGGAGCAGAAGAACATCCTGAGCGAGTTTCAGCGGGACCTGAACGAGTTCGTG |
| | | CTGTGGCTGGAGGAGGCCGATAACATCGCTAGCATCCCCCTGGAGCCCGGCAAGGAGCAGCAGTTA |
| | | AAAGAAAAACTTGAGCAAGTGAAGCTGCTGGTGGAGGAGCTGCCCCTGAGACAAGGCATCCTGA |
| | | AGCAGCTGAACGAGACTGGCGGCCCCGTGCTGGTGAGCGCCCCCATCAGCCCCGAGGAGCAAGAC |
| | | AAGCTGGAGAACAAGCTGAAGCAGACCAACCTGCAGTGGATCAAGGTGAGCAGAGCCCTGCCCG |
| | | AGAAGCAAGGCGAAATCGAGGCCCAAATAAAGGACCTGGGGCAGCTGGAGAAGAAACTCGAAGA |
| | | CTTGGAAGAACAACTCAACCACTTGCTGCTGTGGCTGAGCCCCATCAGAAATCAGCTGGAGATCTA |
| | | CAATCAGCCCAACCAAGAGGGGCCATTCGACGTGAAGGAAACCGAGATCGCCGTGCAAGCCAAGC |
| | | AGCCCGATGTGGAGGAGATCCTGAGCAAGGGGCAGCACCTGTACAAGGAGAAGCCCGCCACACA |
| | | GCCCGTCAAGCGTAAACTAGAGGATCTTTCCTCTGAGTGGAAGGCCGTGAACCGTCTGCTACAAGA |
| | | GCTGCGGGCTAAGCAACCCGACTTAGCGCCCGGCCTGACCACCATCGGTGCAAGCCCCACGCAGA |
| | | CCGTGACCCTGGTGACCCAACCCGTGGTGACCAAGGAAACCGCCATCAGCAAGCTGGAGATGCCT |
| | | AGCAGCCTGATGCTGGAGGTGCCCGCCCTGGCCGACTTCAACAGAGCCTGGACCGAGCTGACCGA |
| | | CTGGCTGAGCCTGCTGGACCAAGTGATCAAGTCTCAGAGAGTGATGGTGGGCGATCTGGAGGACA |
| | | TCAATGAGATGATCATCAAGCAGAAGGCCACCATGCAAGACCTGGAGCAGAGAAGACCTCAGCTG |
| | | GAGGAGCTGATCACCGCCGCTCAGAACCTGAAAAACAAGACGAGCAACCAAGAGGCTAGAACCA |
| | | TCATCACCGACAGAATCGAGAGAATTCAGAATCAGTGGGACGAGGTGCAAGAGCACCTGCAGAAC |
| | | AGAAGACAGCAACTAAACGAAATGCTCAAGGACAGCACACAGTGGCTGGAGGCCAAGGAGGAGG |
| | | CGGAGCAAGTGTTGGGCCAAGCTAGAGCCAAGCTGGAGAGCTGGAAGGAGGGACCCTACACCGT |
| | | GGACGCCATTCAGAAGAAGATCACCGAAACCAAGCAGCTGGCCAAGGACCTGCGACAGTGGCAG |
| | | ACCAACGTGGACGTGGCCAACGATCTTGCCCTGAAGCTGCTGCGCGACTATAGCGCCGACGACAC |
| | | AAGAAAGGTGCACATGATCACCGAGAACATCAACGCTAGCTGGCGTAGCATCCACAAGAGAGTGA |
| | | GCGAGAGAGAGGCGGCTTTGGAGGAAACCCATAGACTGCTGCAGCAGTTTCCCCTGGACCTGGAG |
| | | AAGTTCCTGGCCTGGCTGACCGAAGCCGAAACAACCGCCAACGTGCTGCAAGACGCCACAAGAA |
| | | AGGAGAGACTGCTGGAGGACAGCAAGGGCGTGAAGGAGCTGATGAAGCAGTGGCAAGACCTGCA |
| | | AGGCGAGATCGAAGCGCACACCGACGTGTACCACAACCTGGACGAGAACTCTCAGAAGATCCTGA |
| | | GAAGCCTGGAGGGCAGCGACGACGCCGTGCTGTTACAGAGAAGGCTGGACAACATGAACTTCAAG |
| | | TGGAGCGAGCTGAGAAAGAAGAGCCTGAACATCAGAAGCCACCTGGAGGCTAGCAGCGATCAGT |
| | | GGAAGAGACTGCACCTGAGCCTGCAAGAGCTGCTGGTCTGGCTTCAGTTGAAGGACGATGAGCTG |
| | | AGCAGACAAGCCCCCATCGGCGGCGACTTCCCCGCCGTGCAGAAGCAGAACGACGTGCATAGAGC |
| | | CTTCAAGAGAGAGCTGAAGACCAAGGAGCCCGTGATCATGAGCACCCTGGAAACGGTGAGAATCT |
| | | TCCTGACCGAGCAGCCCCTGGAGGGCCTGGAGAAGCTGTACCAAGAGCCTAGAGAGCTGCCCCCC |
| | | GAGGAGAGAGCTCAGAACGTGACAAGACTGCTGAGAAAGCAAGCCGAAGAGGTGAACACCGAGT |
| | | GGGAGAAGCTGAACCTGCACAGCGCCGACTGGCAGAGAAAGATCGACGAAACCCTGGAGAGACT |
| | | GAGAGAATTGCAAGAGGCGACCGACGAGCTGGACCTGAAGCTGAGACAAGCCGAGGTCATCAAG |
| | | GGCAGCTGGCAGCCCGTGGGCGATCTATTAATTGACTCTCTACAAGACCACCTGGAGAAAGTGAAG |
| | | GCCCTGAGAGGCGAGATCGCCCCCCTGAAGGAGAACGTGAGCCACGTGAATGATCTGGCACGTCA |
| | | GCTGACCACCCTGGGCATACAACTGTCCCCCTATAACCTGAGCACACTGGAGGATTTGAATACTAGA |
| | | TGGAAGCTGCTGCAAGTGGCCGTGGAGGACAGAGTAAGACAATTACATGAGGCCCACAGAGACTT |
| | | CGGCCCCGCTTCTCAGCACTTCCTGAGCACAAGCGTGCAAGGCCCCTGGGAGAGAGCCATTTCCCC |
| | | CAACAAGGTCCCCTACTACATCAACCACGAGACACAGACCACCTGCTGGGACCACCCCAAGATGA |
| | | CCGAGCTGTATCAGAGCCTCGCCGATCTGAACAATGTGAGATTCAGCGCCTACAGAACCGCCATGA |
| | | AGCTGAGAAGACTGCAGAAGGCCCTGTGCCTGGACTTATTAAGCCTGAGCGCCGCCTGCGACGCC |
| | | CTGGATCAGCACAACCTGAAGCAAAACGACCAACCCATGGACATCCTGCAGATCATCAACTGCCTC |
| | | ACCACCATATACGACAGACTGGAACAAGAGCACAACAACCTGGTGAACGTGCCCCTGTGCGTGGA |
| | | CATGTGCCTGAACTGGCTGCTGAACGTGTACGACACCGGCAGAACCGGCAGAATCAGAGTGCTGA |
| | | GCTTCAAGACCGGCATCATCAGCCTGTGCAAGGCCCACCTGGAGGACAAATACAGATACCTTTTCA |
| | | AACAAGTGGCTAGCAGCACCGGCTTCTGCGACCAAAGAAGACTGGGTCTGCTGCTGCACGACAGC |
| | | ATTCAGATCCCTAGACAGCTGGGCGAGGTGGCTAGCTTCGGCGGCAGCAACATCGAGCCTAGCGTG |
| | | AGAAGCTGCTTTCAGTTCGCCAACAACAAGCCCGAGATAGAGGCAGCCCTCTTCTTAGACTGGATG |
| | | AGACTGGAGCCTCAGAGCATGGTATGGCTGCCGGTCCTGCACAGAGTGGCCGCCGCCGAAACCGC |
| | | CAAGCACCAAGCCAAGTGCAACATCTGCAAGGAGTGCCCCATCATCGGCTTCAGATACAGAAGCCT |
| | | GAAGCACTTCAACTACGACATCTGTCAGAGCTGCTTCTTCAGCGGCAGAGTGGCCAAGGGCCACA |
| | | AGATGCACTACCCCATGGTGGAGTACTGCACCCCCACCACAAGCGGCGAGGACGTGAGAGACTTC |
| | | GCCAAGGTGTTGAAGAATAAGTTTAGAACCAAGAGATACTTCGCCAAGCACCCTAGAATGGGCTAC |
| | | CTGCCCGTGCAGACCGTGCTGGAGGGCGACAACATGGAAACCCCCGTGACCCTGATCAACTTCTG |
| | | GCCCGTGGACAGCGCCCCCGCTAGCAGCCCTCAGCTGAGCCACGACGACACCCACAGCAGAATCG |
| | | AGCACTACGCTAGCAGACTGGCCGAGATGGAGAACAGCAACGGCAGCTACCTGAACGACAGCATC |
| | | AGCCCGAATGAGAGCATCGACGACGAGCACCTGCTGATTCAGCACTACTGTCAGAGCCTGAACCA |
| | | AGACAGCCCCCTGTCTCAGCCTAGAAGCCCCGCTCAGATCCTGATCAGCCTGGAGAGCGAGGAGA |
| | | GAGGCGAGCTGGAGAGAATCCTAGCAGATCTGGAGGAAGAGAACAGAAACCTGCAAGCCGAGTAT |
| | | GACCGACTGAAACAGCAACATGAGCACAAAGGCCTGAGCCCACTGCCCTCCCCCCCCGAGATGAT |
| | | GCCCACAAGCCCTCAGAGCCCTAGAGACGCCGAGCTGATCGCTGAAGCGAAGTTACTTAGACAAC |
| | | ACAAGGGCAGACTGGAGGCTAGAATGCAGATCCTGGAGGACCACAACAAGCAGCTGGAGTCTCAG |
| | | CTGCACAGACTGAGGCAGCTGCTGGAACAGCCCCAAGCCGAGGCCAAAGTGAACGGCACCACCG |
| | | TGAGCAGTCCTAGCACAAGCCTGCAGAGAAGCGACAGCTCTCAGCCCATGCTGCTGAGAGTGGTG |
| | | GGCTCTCAGACAAGCGACAGCATGGGCGAGGAGGATCTGCTGAGTCCGCCCCAAGACACAAGCAC |
| | | CGGCCTGGAAGAGGTGATGGAGCAACTGAACAATAGCTTCCCTAGCAGCAGAGGCAGAAACACCC |
| | | CCGGCAAGCCCATGAGAGAGGACACCATGTGATGA |
| 3. | DMD-4 | ATGCTTTGGTGGGAAGAAGTCGAGGACTGCTACGAGCGCGAGGACGTGCAGAAGAAAACCTTCAC |
| | | CAAATGGGTCAACGCCCAGTTCAGCAAGTTCGGCAAGCAGCACATCGAGAACCTGTTCAGCGACC |
| | | TGCAGGATGGCAGAAGGCTGCTGGATCTGCTGGAAGGCCTGACAGGACAGAAGCTGCCCAAAGA |
| | | GAAGGGCAGCACAAGAGTGCACGCCCTGAACAACGTGAACAAGGCCCTGAGAGTGCTGCAGAAC |
| | | AACAACGTGGACCTGGTCAACATCGGCAGCACCGACATCGTGGACGGCAACCACAAACTGACCCT |
| | | GGGCCTGATCTGGAACATCATCCTGCACTGGCAAGTGAAGAACGTGATGAAGAACATCATGGCCGG |
| | | CCTGCAGCAGACCAACAGCGAGAAGATTCTGCTGAGCTGGGTCCGACAGAGCACCCGGAATTACC |
| | | CTCAAGTGAACGTGATCAACTTCACCACCTCTTGGAGCGACGGACTGGCCCTGAATGCCCTGATCC |
| | | ACAGCCACAGACCTGACCTGTTCGACTGGAACAGCGTCGTGTGTCAGCAGAGCGCCACACAGAGG |
| | | CTGGAACACGCCTTCAATATCGCCAGATACCAGCTGGGCATCGAGAAACTGCTGGACCCCGAGGAT |
| | | GTGGACACCACCTATCCTGACAAGAAATCCATCCTCATGTACATCACCAGCCTGTTCCAGGTGCTGC |
| | | CCCAGCAGGTTTCCATCGAGGCCATTCAAGAGGTCGAGATGCTGCCCAGACCTCCTAAAGTGACCA |
| | | AAGAGGAACACTTCCAGCTGCACCACCAGATGCACTACTCTCAGCAGATCACCGTGTCTCTGGCCC |
| | | AGGGCTACGAGAGAACAAGCAGCCCCAAGCCTCGGTTCAAGAGCTACGCCTATACACAGGCCGCC |
| | | TACGTGACCACCAGCGATCCTACAAGAAGCCCATTTCCTAGCCAGCATCTGGAAGCCCCTGAGGAC |
| | | AAGAGCTTTGGCAGCAGCCTGATGGAAAGCGAAGTGAACCTGGACCGCTACCAGACAGCCCTGGA |
| | | AGAGGTTCTGAGCTGGCTGCTGTCTGCCGAGGATACACTGCAGGCTCAGGGCGAGATCAGCAACG |
| | | ACGTGGAAGTGGTCAAGGACCAGTTTCACACCCACGAGGGCTACATGATGGACCTGACAGCCCAC |
| | | CAGGGCAGAGTGGGCAATATTCTGCAGCTGGGCTCCAAGCTGATCGGCACAGGCAAGCTGAGCGA |
| | | GGACGAAGAGACAGAGGTGCAAGAGCAGATGAACCTGCTGAACAGCAGATGGGAGTGTCTGAGA |
| | | GTGGCCAGCATGGAAAAGCAGAGCAACCTGCACCGGGTGCTGATGGATCTCCAGAACCAGAAGCT |
| | | GAAAGAGCTGAACGACTGGCTGACCAAGACCGAGGAACGGACCCGGAAGATGGAAGAGGAACCT |
| | | CTGGGACCCGACCTGGAAGATCTGAAAAGACAGGTGCAGCAGCATAAGGTGCTGCAAGAGGACCT |
| | | CGAACAAGAGCAAGTGCGCGTGAACAGCCTGACACACATGGTGGTGGTCGTGGATGAGAGCAGC |
| | | GGAGATCATGCCACAGCCGCTCTGGAAGAACAGCTGAAGGTGCTGGGAGACAGATGGGCCAATAT |
| | | CTGCCGGTGGACCGAGGATAGATGGGTGCTGCTCCAGGACATCCTGCTGAAGTGGCAGCGGCTGA |
| | | CAGAGGAACAGTGCCTGTTTAGCGCCTGGCTGTCCGAGAAAGAGGACGCCGTCAACAAGATCCAC |
| | | ACCACCGGCTTCAAGGATCAGAATGAGATGCTGAGCAGCCTGCAGAAACTGGCCGTGCTGAAGGC |
| | | TGACCTGGAAAAGAAAAAGCAGTCCATGGGCAAGCTGTACTCCCTGAAGCAGGACCTGCTGAGCA |
| | | CACTGAAGAACAAGTCTGTGACCCAGAAAACCGAGGCCTGGCTGGACAACTTCGCCCGGTGTTGG |
| | | GATAACCTGGTGCAGAAGCTGGAAAAGTCCACCGCTCAGATCTCTCAGGCCGTGACCACAACACA |
| | | GCCTTCTCTGACCCAGACCACCGTGATGGAAACAGTGACCACAGTGACAACCCGCGAGCAGATCC |
| | | TGGTCAAGCACGCCCAAGAAGAACTGCCTCCTCCACCTCCTCAGAAGAAACGGCAGATCACAGTG |
| | | GACAGCGAGATCCGGAAGAGACTGGACGTGGACATCACCGAGCTGCACAGCTGGATCACCAGATC |
| | | CGAAGCCGTGCTGCAGTCCCCTGAGTTCGCCATCTTCAGAAAAGAGGGCAACTTCTCCGACCTGA |
| | | AAGAGAAAGTCAACGCCATCGAGAGAGAGAAGGCCGAGAAGTTCCGGAAGCTGCAGGACGCCTC |
| | | TAGATCTGCCCAGGCTCTGGTGGAACAGATGGTCAACGAAGGCGTGAACGCCGACAGCATCAAGC |
| | | AGGCCTCAGAGCAGCTGAACTCCCGGTGGATCGAGTTCTGTCAGCTCCTGAGCGAGAGACTGAAC |
| | | TGGCTGGAATACCAGAACAATATCATTGCCTTCTACAACCAGCTCCAGCAGCTCGAACAGATGACC |
| | | ACCACAGCCGAGAATTGGCTGAAGATCCAGCCTACCACACCTAGCGAGCCCACCGCCATTAAGAGC |
| | | CAGCTGAAAATCTGCAAGGACGAAGTGAATCGGCTGAGCGATCTGCAGCCCCAGATCGAAAGACT |
| | | GAAAATCCAGTCTATCGCCCTCAAAGAGAAAGGACAGGGCCCCATGTTCCTGGACGCCGATTTTGT |
| | | GGCCTTTACCAACCACTTCAAACAGGTGTTCTCCGACGTGCAGGCCCGGGAAAAAGAGCTGCAGA |
| | | CCATCTTCGACACCCTGCCTCCAATGAGATACCAAGAGACAATGAGCGCCATCCGGACCTGGGTGC |
| | | AGCAAAGCGAGACAAAGCTGAGCATCCCACAGCTGAGCGTGACCGACTACGAGATCATGGAACAG |
| | | AGACTGGGCGAACTGCAGGCCCTCCAGTCTAGCCTGCAAGAACAGCAGTCCGGCCTGTACTACCT |
| | | GAGCACAACCGTGAAAGAGATGAGCAAGAAGGCCCCTAGCGAGATCTCCCGGAAGTACCAGAGC |
| | | GAGTTCGAAGAGATCGAAGGCCGGTGGAAGAAGCTGTCTAGCCAGCTGGTTGAGCACTGCCAGAA |
| | | ACTCGAAGAACAAATGAACAAGCTGCGCAAGATCCAGAATCACATCCAGACGCTGAAGAAATGGA |
| | | TGGCCGAGGTGGACGTGTTCCTGAAAGAAGAGTGGCCCGCTCTGGGCGACTCCGAGATTCTGAAG |
| | | AAACAGCTCAAACAGTGCCGGCTGCTGGTGTCCGATATCCAGACAATCCAGCCAAGCCTGAACTCC |
| | | GTGAATGAAGGCGGCCAGAAGATCAAGAACGAGGCCGAGCCTGAGTTTGCCAGCAGACTGGAAA |
| | | CCGAACTGAAAGAACTCAACACCCAGTGGGACCACATGTGCCAGCAAGTGTACGCCCGGAAAGAG |
| | | GCCCTGAAAGGCGGACTCGAAAAGACTGTGTCTCTGCAGAAAGACCTGTCTGAGATGCACGAGTG |
| | | GATGACCCAGGCCGAAGAGGAATACCTGGAACGGGACTTCGAGTACAAGACCCCTGATGAGCTGC |
| | | AAAAAGCCGTCGAGGAAATGAAGCGGGCCAAAGAAGAGGCCCAGCAGAAAGAAGCCAAAGTCA |
| | | AGCTGCTGACCGAGTCCGTGAATAGCGTTATCGCTCAGGCCCCTCCTGTGGCTCAAGAGGCTCTCA |
| | | AGAAAGAACTGGAAACTCTGACCACCAACTACCAGTGGCTGTGCACCAGACTGAACGGCAAGTGC |
| | | AAGACACTGGAAGAAGTGTGGGCCTGCTGGCACGAACTGCTGTCCTATCTGGAAAAGGCCAACAA |
| | | GTGGCTGAACGAGGTGGAATTCAAGCTGAAAACCACCGAGAACATCCCTGGCGGCGCTGAAGAGA |
| | | TTAGCGAGGTGCTGGACAGCCTGGAAAACCTGATGAGACACAGCGAGGACAACCCCAATCAGATC |
| | | AGAATCCTGGCTCAGACCCTGACCGATGGCGGCGTGATGGACGAGCTGATCAACGAGGAACTCGA |
| | | AACCTTCAACAGCCGGTGGCGGGAACTGCACGAGGAAGCTGTTCGGAGGCAGAAGTTGCTGGAA |
| | | CAGTCTATCCAGAGCGCACAAGAAACCGAGAAGTCCCTGCACCTGATCCAAGAGAGCCTGACCTT |
| | | CATCGACAAGCAGCTGGCCGCTTATATCGCCGACAAGGTGGACGCTGCTCAGATGCCCCAAGAGGC |
| | | ACAGAAGATTCAGAGCGACCTGACCAGCCACGAGATTAGCCTGGAAGAAATGAAGAAGCACAACC |
| | | AGGGCAAAGAGGCCGCTCAGAGAGTCCTGAGCCAGATCGATGTGGCACAGAAAAAGCTCCAGGAT |
| | | GTGTCCATGAAGTTTCGGCTGTTTCAGAAGCCCGCCAACTTCGAGCAGAGACTGCAAGAGTCCAA |
| | | GATGATCCTGGACGAAGTCAAAATGCATCTGCCCGCACTGGAAACAAAGTCCGTGGAACAAGAAG |
| | | TGGTGCAGTCCCAGCTGAACCACTGCGTGAACCTGTACAAGAGCCTGTCCGAAGTGAAGTCTGAG |
| | | GTGGAAATGGTCATCAAGACCGGCAGGCAGATCGTCCAGAAGAAGCAGACCGAGAATCCCAAAGA |
| | | ACTCGACGAGAGAGTGACCGCTCTGAAGCTGCACTACAATGAGCTGGGCGCCAAAGTGACCGAGC |
| | | GGAAGCAACAGCTTGAGAAGTGCCTGAAACTGTCTCGGAAGATGCGGAAAGAAATGAACGTGCTG |
| | | ACAGAGTGGCTGGCCGCCACCGATATGGAACTGACTAAGAGAAGCGCCGTGGAAGGCATGCCCAG |
| | | CAACCTGGATAGTGAAGTGGCCTGGGGCAAAGCCACTCAGAAAGAGATTGAGAAGCAGAAGGTCC |
| | | ACCTGAAGTCCATCACCGAAGTGGGCGAAGCCCTGAAAACCGTGCTGGGAAAGAAAGAGACACT |
| | | GGTCGAGGACAAGCTGTCCCTGCTGAATTCCAACTGGATCGCCGTGACCTCCAGAGCTGAGGAATG |
| | | GCTGAATCTGCTGCTTGAGTACCAGAAACACATGGAAACGTTCGACCAGAACGTCGACCACATCAC |
| | | AAAGTGGATCATCCAGGCTGATACCCTGCTGGACGAGTCCGAGAAGAAGAAACCCCAACAAAAAG |
| | | AAGATGTGCTGAAGCGGCTGAAAGCCGAGCTGAATGACATCCGGCCTAAGGTGGACAGCACCAGA |
| | | GATCAGGCAGCCAACCTGATGGCCAACAGAGGCGACCACTGTCGGAAGCTCGTGGAACCTCAGAT |
| | | CAGCGAGCTTAACCACAGATTTGCCGCCATCAGCCACCGGATCAAGACAGGCAAGGCCAGCATTCC |
| | | TCTCAAAGAGCTTGAGCAGTTCAACAGCGACATCCAAAAGCTGCTCGAACCCCTGGAAGCCGAGA |
| | | TCCAACAGGGCGTCAACCTCAAAGAAGAAGATTTCAACAAGGACATGAACGAGGACAATGAGGG |
| | | CACCGTCAAAGAACTGCTGCAGCGGGGCGATAATCTGCAGCAGCGGATTACCGATGAGCGCAAGC |
| | | GGGAAGAGATCAAGATTAAGCAGCAGCTGCTGCAGACCAAGCACAACGCCCTGAAGGACCTGCGG |
| | | AGCCAGAGAAGAAAGAAGGCCCTGGAAATCAGCCACCAGTGGTATCAGTACAAGCGGCAGGCCG |
| | | ACGACCTGCTGAAGTGCCTGGATGACATCGAGAAGAAGCTGGCCTCTCTGCCCGAGCCTAGGGAC |
| | | GAGAGAAAGATCAAAGAGATCGACCGCGAGCTGCAGAAAAAGAAAGAGGAACTGAACGCCGTCC |
| | | GCAGACAGGCCGAAGGACTTTCTGAAGATGGCGCCGCTATGGCCGTGGAACCCACACAGATTCAG |
| | | CTGAGCAAGCGGTGGCGGGAAATCGAGAGCAAGTTCGCCCAGTTCCGGCGGCTGAATTTCGCCCA |
| | | GATCCACACCGTGCGGGAAGAGACAATGATGGTCATGACAGAGGACATGCCCCTTGAGATCAGCTA |
| | | CGTGCCCAGCACCTACCTGACCGAGATCACCCATGTGTCTCAGGCCCTGCTGGAAGTGGAACAGCT |
| | | GCTGAATGCCCCTGACCTGTGCGCCAAGGACTTCGAGGACCTGTTCAAGCAAGAGGAAAGCCTGA |
| | | AGAACATCAAGGACAGCCTGCAGCAGAGCAGCGGCCGGATCGATATCATCCACAGCAAGAAAACC |
| | | GCCGCACTGCAGAGCGCCACACCTGTGGAAAGAGTGAAGCTGCAAGAGGCCCTGAGCCAGCTGG |
| | | ATTTCCAGTGGGAGAAAGTGAACAAGATGTACAAGGACCGGCAGGGCAGATTCGACCGCAGCGTT |
| | | GAAAAGTGGCGGCGGTTCCACTACGACATCAAGATCTTCAACCAGTGGCTGACCGAGGCCGAGCA |
| | | GTTCCTGAGAAAGACACAGATCCCCGAGAACTGGGAGCACGCCAAGTACAAGTGGTATCTGAAAG |
| | | AGCTGCAGGACGGCATCGGCCAGAGACAGACAGTCGTGCGGACACTGAATGCCACCGGCGAGGA |
| | | AATCATCCAGCAGTCCAGCAAGACCGACGCCAGCATCCTGCAAGAGAAGCTGGGCAGCCTGAACC |
| | | TGCGGTGGCAAGAAGTGTGCAAGCAGCTGAGCGACCGGAAGAAGCGGCTGGAAGAACAGAAGA |
| | | ATATCCTGAGCGAGTTCCAGCGGGACCTGAACGAGTTCGTGCTGTGGCTCGAAGAGGCCGACAATA |
| | | TCGCCAGCATTCCCCTGGAACCTGGCAAAGAGCAGCAGCTGAAAGAAAAGCTGGAACAAGTGAA |
| | | ACTGCTGGTCGAGGAACTGCCCCTGAGACAGGGAATCCTGAAACAGCTGAACGAGACAGGCGGC |
| | | CCTGTGCTGGTGTCTGCCCCTATTTCTCCCGAGGAACAGGACAAGCTGGAAAACAAGCTGAAGCA |
| | | GACCAACCTGCAGTGGATCAAGGTGTCCAGAGCACTGCCTGAGAAGCAGGGCGAGATCGAGGCCC |
| | | AGATCAAGGACCTGGGACAGCTCGAAAAGAAGCTTGAGGACCTCGAAGAACAGCTCAACCATCTG |
| | | CTGCTTTGGCTGAGCCCCATCCGGAACCAGCTGGAAATCTACAACCAGCCTAATCAAGAGGGCCCC |
| | | TTCGACGTGAAAGAAACCGAGATTGCCGTGCAGGCCAAGCAGCCCGATGTGGAAGAGATCCTGTC |
| | | TAAGGGCCAGCACCTGTACAAAGAGAAGCCCGCCACACAGCCCGTGAAGCGGAAACTGGAAGAT |
| | | CTGAGCAGCGAGTGGAAGGCCGTGAACCGGCTGCTGCAAGAACTGAGAGCCAAACAGCCTGATCT |
| | | GGCCCCTGGCCTGACAACAATTGGCGCCTCTCCTACACAGACCGTGACACTGGTCACACAGCCTGT |
| | | GGTCACCAAAGAGACAGCCATCAGCAAACTGGAAATGCCCAGCAGTCTGATGCTCGAAGTGCCCG |
| | | CACTGGCCGACTTCAATAGAGCCTGGACCGAGCTGACCGACTGGCTCAGTCTGCTGGACCAAGTC |
| | | ATCAAGTCCCAGAGAGTGATGGTCGGAGATCTTGAGGACATCAACGAGATGATCATTAAGCAGAAA |
| | | GCCACCATGCAGGACCTTGAGCAGAGAAGGCCCCAGCTTGAAGAACTGATCACCGCCGCTCAGAA |
| | | CCTGAAAAACAAGACCAGCAATCAAGAAGCCCGGACCATCATCACCGACCGGATCGAGAGAATCC |
| | | AGAACCAGTGGGACGAAGTGCAAGAGCATCTGCAGAACAGACGGCAGCAGCTCAATGAGATGCT |
| | | GAAGGACAGCACACAGTGGCTGGAAGCCAAAGAAGAAGCCGAGCAGGTTCTCGGACAGGCCAGA |
| | | GCTAAGCTGGAATCTTGGAAAGAGGGACCCTACACCGTCGACGCCATCCAGAAGAAGATCACCGA |
| | | GACAAAGCAGCTGGCCAAGGATCTGAGACAGTGGCAGACCAATGTGGACGTGGCCAACGACCTG |
| | | GCTCTGAAGCTGCTGAGAGACTACAGCGCCGACGACACCAGAAAGGTGCACATGATCACCGAAAA |
| | | CATCAACGCCTCTTGGCGGAGCATCCACAAGCGGGTGTCAGAGAGAGAAGCCGCTCTGGAAGAAA |
| | | CACATAGACTGCTCCAGCAGTTCCCACTCGACCTGGAAAAGTTCCTGGCCTGGCTGACAGAAGCCG |
| | | AAACCACCGCTAACGTGCTCCAGGATGCCACCAGAAAAGAGCGGCTGTTGGAGGACAGCAAGGG |
| | | CGTCAAAGAACTCATGAAGCAGTGGCAGGATCTGCAAGGGGAGATTGAAGCCCACACCGACGTGT |
| | | ACCACAACCTGGACGAGAACAGCCAGAAGATCCTGCGGTCCCTGGAAGGCTCTGATGATGCTGTG |
| | | CTGCTTCAGAGGCGGCTGGACAACATGAACTTCAAGTGGAGCGAGCTGCGGAAGAAGTCCCTGAA |
| | | CATCAGAAGCCACCTGGAAGCCTCCAGCGACCAGTGGAAAAGACTGCACCTGTCTCTCCAAGAGC |
| | | TGCTCGTGTGGCTGCAGCTGAAGGATGACGAGCTGAGTAGACAGGCCCCTATCGGCGGAGATTTTC |
| | | CCGCCGTGCAGAAACAGAACGACGTGCACCGGGCCTTCAAGAGAGAGCTGAAAACAAAAGAACC |
| | | CGTCATCATGAGCACCCTGGAAACCGTGCGGATCTTTCTGACCGAGCAGCCTCTGGAAGGACTGGA |
| | | AAAGCTGTACCAAGAGCCTAGAGAGCTGCCTCCTGAAGAAAGGGCCCAGAACGTGACACGGCTGC |
| | | TTAGAAAGCAGGCCGAGGAAGTGAACACCGAATGGGAGAAGCTGAACCTCCACTCCGCCGACTGG |
| | | CAGCGGAAGATCGATGAGACACTGGAACGGCTGCGGGAACTGCAAGAAGCCACAGACGAGCTGG |
| | | ACCTGAAACTGCGGCAGGCTGAAGTGATCAAAGGCTCCTGGCAGCCAGTGGGCGATCTGCTGATC |
| | | GATAGCCTGCAGGACCATCTGGAAAAAGTCAAGGCCCTGCGGGGAGAGATCGCCCCTCTCAAAGA |
| | | AAACGTGTCCCACGTGAACGATCTGGCCAGGCAGCTGACAACACTGGGCATCCAGCTGAGCCCTT |
| | | ACAACCTGTCTACCTTGGAGGACCTGAATACCCGGTGGAAGCTGCTCCAAGTGGCCGTCGAGGATA |
| | | GAGTGCGGCAGCTGCATGAGGCTCACAGAGATTTTGGACCCGCCAGCCAGCACTTCCTGAGCACAT |
| | | CTGTTCAAGGCCCCTGGGAGAGAGCTATCAGCCCTAACAAGGTGCCCTACTACATCAACCACGAGA |
| | | CACAGACCACCTGTTGGGATCACCCCAAGATGACCGAACTGTATCAGTCCCTGGCCGACCTGAACA |
| | | ACGTGCGGTTTAGCGCCTACCGGACCGCCATGAAGCTGCGCAGACTGCAGAAAGCTCTGTGCCTG |
| | | GACCTGCTGTCTCTGAGCGCTGCTTGTGATGCCCTGGACCAGCATAACCTCAAGCAGAACGACCAG |
| | | CCTATGGACATCCTGCAGATCATCAACTGCCTGACCACCATCTACGACCGGCTCGAACAAGAGCAC |
| | | AACAACCTGGTCAACGTGCCCCTGTGCGTGGACATGTGCCTGAATTGGCTGCTGAACGTGTACGAC |
| | | ACCGGCAGAACCGGCAGGATCAGAGTGCTGAGCTTCAAGACCGGCATCATCTCCCTGTGCAAGGC |
| | | CCACCTTGAGGATAAGTACCGCTACCTGTTTAAGCAGGTCGCCAGCAGCACCGGCTTTTGCGACCA |
| | | AAGAAGGCTGGGACTGCTGCTCCACGACAGCATTCAGATCCCTAGACAGCTGGGCGAAGTGGCCT |
| | | CTTTCGGCGGCTCTAATATCGAGCCTAGCGTGCGGAGCTGCTTCCAGTTCGCCAACAACAAGCCCG |
| | | AGATTGAGGCCGCACTGTTCCTGGACTGGATGAGACTGGAACCCCAGAGCATGGTCTGGCTGCCTG |
| | | TGCTGCATAGAGTGGCCGCAGCCGAAACAGCCAAGCACCAGGCCAAGTGCAACATCTGCAAAGAG |
| | | TGCCCCATCATCGGCTTCCGGTACAGATCCCTGAAGCACTTCAACTACGATATCTGCCAGTCCTGCT |
| | | TCTTCAGCGGCAGAGTGGCCAAGGGCCACAAGATGCACTACCCCATGGTGGAATACTGCACCCCTA |
| | | CCACCTCTGGCGAGGACGTGCGGGATTTTGCCAAGGTGCTCAAGAACAAGTTCCGGACCAAGCGC |
| | | TACTTCGCTAAGCACCCCAGAATGGGCTACCTGCCAGTGCAGACAGTGCTTGAGGGCGACAACATG |
| | | GAAACCCCTGTGACTCTGATCAACTTCTGGCCCGTGGATAGCGCCCCTGCTAGTTCTCCTCAGCTGT |
| | | CTCACGACGATACCCACAGCAGAATCGAGCACTACGCCTCCAGACTGGCCGAGATGGAAAACAGC |
| | | AACGGCAGCTACCTGAATGACAGCATCAGCCCCAACGAGAGCATCGACGACGAACATCTGCTCATT |
| | | CAGCACTACTGCCAGAGCCTGAATCAGGACAGCCCTCTGTCTCAGCCTAGAAGCCCTGCACAGATC |
| | | CTGATCAGCCTGGAAAGCGAGGAACGCGGCGAGCTGGAAAGAATCCTGGCAGACCTGGAAGAGG |
| | | AAAACCGGAACCTGCAGGCCGAATACGACAGACTCAAGCAGCAGCACGAGCACAAGGGACTGTC |
| | | TCCACTGCCTTCTCCACCTGAAATGATGCCCACCTCTCCACAGAGCCCCAGAGATGCCGAGCTGAT |
| | | TGCCGAAGCCAAACTGCTGAGGCAGCACAAAGGCAGACTCGAAGCCAGAATGCAGATTCTCGAAG |
| | | ATCACAACAAGCAGCTCGAATCCCAGCTGCACAGACTTAGGCAGCTGCTGGAACAGCCTCAGGCA |
| | | GAGGCCAAAGTGAATGGCACCACCGTGTCTAGCCCTAGCACAAGCCTGCAGAGATCCGACAGCAG |
| | | CCAGCCAATGCTTCTGAGAGTCGTGGGCTCCCAGACCAGCGATTCTATGGGCGAAGAGGACCTGCT |
| | | CAGCCCTCCTCAGGATACAAGCACCGGCCTGGAAGAAGTGATGGAACAACTGAACAACAGCTTCC |
| | | CCAGCAGCAGAGGCAGAAACACCCCTGGCAAGCCCATGCGCGAGGACACCATGTGATAA |
| 4. | MHCK | CTAGAAGCTGCATGTCTAAGCTAGACCCTTCAGATTAAAAATAACTGAGGTAAGGGCCTGGGTAGG |
| | Promoter | GGAGGTGGTGTGAGACGCTCCTGTCTCTCCTCTATCTGCCCATCGGCCCTTTGGGGAGGAGGAATG |
| | | TGCCCAAGGACTAAAAAAAGGCCATGGAGCCAGAGGGGCGAGGGCAACAGACCTTTCATGGGCA |
| | | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACC |
| 5. | MHCK | TGCCCAAGGACTAAAAAAAGGCCATGGAGCCAGAGGGGCGAGGGCAACAGACCTTTCATGGGCA |
| | Promoter SF | CTAGAAGCTGCATGTCTAAGCTAGACCCTTCAGATTAAAAATAACTGAGGTAAGGGCCTGGGTAGG |
| | | GGAGGTGGTGTGAGACGCTCCTGTCTCTCCTCTATCTGCCCATCGGCCCTTTGGGGAGGAGGAATG |
| | | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| 6. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 3-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTG |
| | | TGGTGGGAAGAGGTGGAGGACTGTTACGAGAGAGAGGACGTGCAGAAGAAGACATTCACCAAGT |
| | | GGGTGAACGCTCAGTTCAGCAAGTTCGGCAAGCAGCACATCGAGAACCTGTTTAGCGATCTACAA |
| | | GATGGCAGAAGACTGCTGGACCTGCTGGAGGGCCTGACCGGGCAGAAGCTGCCCAAAGAAAAGG |
| | | GGTCTACAAGAGTGCACGCCCTGAACAACGTGAACAAGGCCCTGAGAGTGCTGCAGAACAACAA |
| | | CGTGGACCTGGTGAACATCGGCAGCACCGACATCGTGGACGGCAACCACAAGCTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATCCTGCTGAGCTGGGTGAGACAGAGCACAAGAAACTACCCCCA |
| | | AGTGAACGTGATCAACTTCACCACAAGCTGGAGCGACGGCCTGGCCCTGAACGCCCTGATCCACA |
| | | GCCACAGACCCGACCTGTTCGACTGGAACAGCGTGGTGTGTCAGCAGAGCGCCACACAGAGACTG |
| | | GAGCACGCCTTCAACATCGCTAGATATCAGCTGGGCATCGAGAAGTTACTGGACCCCGAAGATGTG |
| | | GACACCACCTACCCCGACAAGAAGAGCATCCTGATGTACATCACAAGCCTGTTCCAAGTGCTGCCT |
| | | CAGCAAGTGAGCATCGAGGCCATCCAAGAAGTCGAGATGCTGCCTAGACCCCCCAAGGTGACCAA |
| | | GGAGGAGCACTTTCAGCTGCACCATCAGATGCACTACTCTCAGCAGATCACAGTCAGCTTGGCACA |
| | | AGGCTACGAGAGAACAAGCAGCCCCAAGCCTAGATTCAAGAGCTACGCCTACACCCAAGCCGCCT |
| | | ACGTGACCACAAGTGACCCCACAAGAAGCCCCTTCCCTTCTCAGCACCTGGAGGCCCCCGAGGAC |
| | | AAGAGCTTCGGCAGCAGCCTGATGGAGAGCGAGGTGAACCTGGACAGATATCAGACCGCGTTAGA |
| | | AGAAGTGTTGAGCTGGCTGCTGAGCGCCGAGGACACCCTGCAAGCCCAAGGCGAGATCAGCAAC |
| | | GATGTGGAGGTGGTGAAGGATCAGTTCCACACCCACGAGGGCTACATGATGGACCTGACCGCCCAC |
| | | CAAGGCAGAGTGGGCAACATCCTGCAGCTGGGCAGCAAGCTGATCGGCACCGGCAAGCTGAGCG |
| | | AGGACGAGGAAACCGAGGTTCAAGAGCAAATGAACCTGCTAAACAGCAGATGGGAGTGCCTGAG |
| | | AGTGGCTAGCATGGAAAAGCAGAGTAACCTGCACAGAGTGCTGATGGACCTGCAGAATCAGAAAT |
| | | TGAAAGAACTTAATGATTGGCTGACCAAGACCGAGGAGAGAACAAGAAAGATGGAGGAGGAGCC |
| | | CCTGGGCCCCGACCTGGAGGACCTGAAGAGACAAGTGCAGCAGCACAAGGTGCTGCAAGAGGAC |
| | | CTGGAGCAAGAGCAAGTGAGAGTGAACTCTCTGACACACATGGTGGTGGTAGTGGACGAGAGCAG |
| | | CGGCGATCACGCTACCGCGGCGCTGGAAGAGCAGCTGAAAGTGCTGGGCGACAGATGGGCCAACA |
| | | TCTGCAGATGGACCGAGGACAGATGGGTGCTGCTGCAAGACATCCTGCTGAAGTGGCAGAGACTG |
| | | ACCGAGGAGCAGTGCCTGTTCAGCGCCTGGCTGAGCGAGAAGGAAGACGCTGTGAACAAGATCC |
| | | ACACCACCGGCTTCAAGGATCAGAACGAGATGCTGAGCTCCCTCCAAAAGCTCGCAGTGCTGAAG |
| | | GCCGACCTGGAAAAGAAAAAACAGTCGATGGGCAAGCTGTACAGCCTGAAGCAAGACCTGCTGA |
| | | GCACCCTGAAGAACAAGAGCGTGACACAGAAGACCGAGGCCTGGCTGGACAACTTCGCTAGATGC |
| | | TGGGACAACCTGGTACAGAAGTTAGAGAAGTCTACCGCTCAGATCAGCCAAGCCGTAACCACCAC |
| | | ACAACCTAGCCTGACTCAGACCACCGTGATGGAAACCGTGACCACCGTGACAACCCGTGAGCAGA |
| | | TCCTGGTGAAGCACGCCCAAGAGGAGCTGCCCCCCCCCCCCCCTCAGAAGAAGAGACAGATCACC |
| | | GTGGACTCAGAGATTCGCAAGAGACTGGACGTGGACATCACCGAGCTGCACAGCTGGATCACAAG |
| | | AAGCGAGGCCGTGCTGCAGAGCCCTGAGTTCGCGATCTTTAGAAAGGAGGGCAACTTCAGCGACT |
| | | TGAAGGAGAAAGTGAACGCCATCGAGAGAGAGAAGGCCGAGAAGTTCAGAAAGTTGCAAGACGC |
| | | CTCAAGAAGCGCCCAAGCCCTGGTGGAGCAGATGGTTAACGAAGGCGTGAATGCCGACAGCATCA |
| | | AGCAAGCTAGCGAGCAGCTGAATAGCCGGTGGATTGAATTCTGTCAGCTGCTGAGCGAGAGATTAA |
| | | ACTGGCTGGAGTATCAGAACAACATCATCGCCTTCTACAATCAGCTGCAGCAACTAGAACAAATGA |
| | | CCACCACCGCCGAGAACTGGCTAAAGATTCAGCCCACCACCCCTAGCGAGCCCACCGCCATCAAAT |
| | | CTCAGCTGAAAATTTGCAAGGACGAAGTGAACAGACTGTCGGACCTGCAGCCTCAAATCGAAAGA |
| | | CTGAAAATTCAGAGCATCGCACTTAAGGAGAAGGGCCAAGGACCCATGTTCCTGGACGCCGACTTC |
| | | GTGGCCTTCACCAACCACTTCAAGCAAGTGTTCAGCGACGTGCAAGCTAGAGAGAAGGAGCTGCA |
| | | GACCATCTTCGACACCCTGCCCCCCATGAGATACCAAGAAACCATGAGCGCCATCAGAACCTGGGT |
| | | GCAGCAGAGCGAAACCAAGCTGAGCATCCCTCAGCTGAGCGTGACCGACTACGAGATCATGGAGC |
| | | AAAGACTGGGAGAGCTGCAAGCCCTGCAGAGCAGCCTGCAAGAGCAGCAGAGCGGCCTGTACTA |
| | | CCTGAGCACCACCGTGAAGGAGATGTCGAAGAAAGCCCCGTCGGAGATCAGCAGAAAGTATCAGA |
| | | GCGAGTTCGAGGAGATCGAGGGCAGATGGAAGAAGCTGAGCAGCCAACTGGTGGAACACTGTCA |
| | | GAAATTAGAAGAACAGATGAACAAGCTGAGAAAGATTCAGAACCACATTCAGACCCTGAAGAAGT |
| | | GGATGGCCGAGGTGGACGTGTTCCTGAAAGAGGAGTGGCCCGCCCTGGGCGATTCCGAAATCTTA |
| | | AAAAAGCAGCTGAAGCAGTGCAGACTGCTGGTGTCAGATATCCAAACCATTCAACCAAGCCTGAA |
| | | CAGCGTGAACGAAGGCGGGCAGAAGATCAAGAACGAGGCCGAGCCCGAGTTCGCTAGCAGACTG |
| | | GAAACCGAGCTGAAGGAGCTGAATACACAGTGGGACCACATGTGTCAGCAAGTGTACGCTAGAAA |
| | | GGAAGCGCTGAAGGGCGGGCTGGAGAAGACCGTTAGCCTGCAAAAGGATCTGAGCGAGATGCAC |
| | | GAGTGGATGACCCAAGCCGAGGAGGAGTACCTGGAGAGAGACTTCGAGTACAAGACCCCCGACG |
| | | AGCTGCAGAAGGCCGTCGAGGAAATGAAGAGAGCGAAGGAGGAGGCTCAGCAGAAGGAAGCCA |
| | | AGGTGAAGCTGCTGACTGAATCTGTCAACAGTGTGATAGCTCAAGCACCCCCCGTTGCCCAAGAGG |
| | | CACTAAAGAAGGAGCTGGAAACTCTGACCACCAACTATCAGTGGCTGTGCACAAGACTGAACGGC |
| | | AAGTGCAAGACCCTGGAGGAAGTGTGGGCCTGCTGGCACGAGCTGCTGAGCTACCTGGAGAAGGC |
| | | CAACAAGTGGCTGAACGAGGTGGAGTTCAAGCTGAAGACCACCGAGAACATCCCCGGCGGCGCC |
| | | GAGGAGATCAGCGAGGTGCTGGACAGCCTGGAGAACCTGATGAGACACAGCGAGGACAACCCCA |
| | | ATCAGATCAGAATCCTGGCTCAGACCCTGACCGACGGCGGCGTGATGGACGAGCTGATCAACGAG |
| | | GAGCTGGAAACCTTTAACTCTAGATGGCGAGAACTGCACGAGGAGGCCGTGAGAAGACAGAAGCT |
| | | GCTGGAGCAAAGCATTCAGAGCGCCCAAGAAACCGAGAAGAGCCTGCACCTGATCCAAGAGAGC |
| | | CTGACCTTCATCGACAAGCAGCTGGCCGCCTACATCGCCGACAAGGTGGACGCCGCTCAGATGCCA |
| | | CAAGAGGCTCAGAAGATTCAGTCCGATCTGACAAGCCACGAGATCAGCCTGGAGGAGATGAAAAA |
| | | ACACAACCAAGGCAAGGAGGCCGCTCAGAGAGTGCTGTCTCAGATCGACGTGGCTCAGAAGAAG |
| | | CTGCAAGACGTGAGCATGAAGTTCAGACTGTTTCAGAAGCCCGCCAACTTCGAGCAGCGACTGCA |
| | | AGAGAGCAAGATGATCCTGGACGAGGTGAAGATGCACCTGCCCGCCCTGGAAACCAAGAGCGTGG |
| | | AGCAAGAGGTGGTGCAGTCTCAGTTGAACCACTGCGTGAACCTGTACAAGAGCCTGTCGGAGGTC |
| | | AAGAGTGAGGTCGAGATGGTGATCAAAACTGGGAGACAGATCGTGCAAAAAAAGCAGACCGAGA |
| | | ACCCCAAGGAGCTGGACGAGCGTGTTACCGCCCTGAAGCTGCACTACAACGAGCTGGGCGCCAAG |
| | | GTGACCGAGAGAAAGCAGCAGCTGGAGAAGTGCCTGAAGCTGAGCAGAAAGATGAGAAAGGAG |
| | | ATGAACGTGCTGACCGAGTGGCTGGCCGCCACCGACATGGAGCTGACCAAGAGAAGCGCCGTGGA |
| | | GGGCATGCCTAGCAACCTGGACAGCGAGGTGGCCTGGGGCAAGGCCACACAGAAGGAGATCGAG |
| | | AAGCAGAAGGTGCACCTGAAGAGCATCACCGAGGTGGGCGAGGCCCTGAAGACCGTGCTGGGCA |
| | | AGAAGGAAACCCTGGTGGAGGACAAGCTGAGCCTGCTGAACTCGAACTGGATCGCCGTGACAAG |
| | | CAGAGCCGAGGAGTGGCTGAACCTGCTGTTAGAGTACCAAAAACACATGGAAACCTTCGATCAGA |
| | | ACGTGGACCACATCACCAAGTGGATCATCCAAGCCGACACCCTGCTGGACGAAAGCGAAAAGAAG |
| | | AAGCCGCAACAAAAGGAGGATGTGCTGAAGAGACTGAAGGCCGAACTGAACGATATCAGACCCA |
| | | AGGTGGACAGCACACGGGACCAAGCCGCCAACCTGATGGCCAACAGAGGCGACCACTGCAGAAA |
| | | ACTGGTGGAGCCTCAGATCAGCGAGCTGAACCACAGATTCGCCGCCATCAGCCACAGAATCAAAA |
| | | CCGGCAAAGCGAGCATCCCCTTGAAGGAACTGGAGCAATTTAACAGCGACATCCAAAAATTGCTCG |
| | | AACCACTGGAGGCCGAGATTCAGCAAGGCGTGAACCTGAAGGAAGAGGACTTCAACAAGGACAT |
| | | GAACGAGGACAACGAGGGCACCGTGAAAGAACTGCTGCAGAGAGGCGACAACCTGCAGCAGAG |
| | | AATCACCGACGAGAGAAAGAGAGAGGAGATCAAGATCAAGCAGCAGCTGCTGCAGACTAAGCAC |
| | | AACGCCCTGAAGGATCTGAGATCTCAGAGGAGAAAAAAGGCTCTGGAAATTTCACATCAGTGGTAT |
| | | CAGTACAAGAGACAAGCAGACGACCTGCTGAAGTGCCTGGACGACATCGAGAAGAAGTTAGCTAG |
| | | CCTGCCCGAGCCTAGAGACGAGAGAAAGATCAAGGAGATCGACAGAGAGTTACAAAAGAAGAAG |
| | | GAGGAGCTGAATGCGGTAAGAAGACAAGCGGAAGGCCTGTCCGAGGACGGCGCAGCCATGGCCG |
| | | TGGAGCCCACACAGATTCAGCTCAGCAAGAGATGGAGAGAGATCGAGAGCAAGTTCGCTCAGTTC |
| | | AGAAGACTGAACTTCGCTCAGATCCACACCGTGAGAGAGGAAACCATGATGGTGATGACCGAGGA |
| | | CATGCCCCTGGAGATCTCATACGTTCCTAGCACCTACCTGACCGAGATCACCCACGTGAGCCAAGCC |
| | | CTGCTGGAGGTGGAGCAGCTGCTGAACGCCCCCGACCTGTGCGCCAAGGACTTCGAGGACCTGTT |
| | | CAAACAAGAGGAGAGCCTGAAGAACATCAAGGACAGCCTGCAGCAAAGTAGCGGCAGAATCGAC |
| | | ATCATCCACAGCAAGAAGACCGCCGCCCTGCAGAGCGCCACCCCCGTGGAGAGAGTGAAGCTTCA |
| | | AGAGGCCCTTTCGCAGCTGGATTTTCAGTGGGAGAAGGTGAACAAAATGTACAAGGACAGACAAG |
| | | GCAGATTCGACAGAAGCGTGGAGAAGTGGAGAAGATTCCACTACGACATCAAGATCTTCAATCAGT |
| | | GGCTGACCGAGGCAGAGCAGTTCCTGAGAAAGACACAGATCCCCGAGAACTGGGAGCACGCCAA |
| | | GTACAAGTGGTACCTGAAGGAGCTGCAAGACGGCATCGGGCAGAGACAGACCGTGGTGAGAACC |
| | | CTGAACGCCACCGGCGAGGAGATCATCCAACAAAGCTCCAAAACCGACGCTAGCATCCTGCAAGA |
| | | GAAACTGGGCAGCCTGAACCTGAGATGGCAAGAGGTGTGCAAGCAGCTGAGCGACCGCAAAAAA |
| | | CGGTTAGAGGAGCAGAAGAACATCCTGAGCGAGTTTCAGCGGGACCTGAACGAGTTCGTGCTGTG |
| | | GCTGGAGGAGGCCGATAACATCGCTAGCATCCCCCTGGAGCCCGGCAAGGAGCAGCAGTTAAAAG |
| | | AAAAACTTGAGCAAGTGAAGCTGCTGGTGGAGGAGCTGCCCCTGAGACAAGGCATCCTGAAGCA |
| | | GCTGAACGAGACTGGCGGCCCCGTGCTGGTGAGCGCCCCCATCAGCCCCGAGGAGCAAGACAAGC |
| | | TGGAGAACAAGCTGAAGCAGACCAACCTGCAGTGGATCAAGGTGAGCAGAGCCCTGCCCGAGAA |
| | | GCAAGGCGAAATCGAGGCCCAAATAAAGGACCTGGGGCAGCTGGAGAAGAAACTCGAAGACTTG |
| | | GAAGAACAACTCAACCACTTGCTGCTGTGGCTGAGCCCCATCAGAAATCAGCTGGAGATCTACAAT |
| | | CAGCCCAACCAAGAGGGGCCATTCGACGTGAAGGAAACCGAGATCGCCGTGCAAGCCAAGCAGC |
| | | CCGATGTGGAGGAGATCCTGAGCAAGGGGCAGCACCTGTACAAGGAGAAGCCCGCCACACAGCCC |
| | | GTCAAGCGTAAACTAGAGGATCTTTCCTCTGAGTGGAAGGCCGTGAACCGTCTGCTACAAGAGCTG |
| | | CGGGCTAAGCAACCCGACTTAGCGCCCGGCCTGACCACCATCGGTGCAAGCCCCACGCAGACCGT |
| | | GACCCTGGTGACCCAACCCGTGGTGACCAAGGAAACCGCCATCAGCAAGCTGGAGATGCCTAGCA |
| | | GCCTGATGCTGGAGGTGCCCGCCCTGGCCGACTTCAACAGAGCCTGGACCGAGCTGACCGACTGG |
| | | CTGAGCCTGCTGGACCAAGTGATCAAGTCTCAGAGAGTGATGGTGGGCGATCTGGAGGACATCAAT |
| | | GAGATGATCATCAAGCAGAAGGCCACCATGCAAGACCTGGAGCAGAGAAGACCTCAGCTGGAGGA |
| | | GCTGATCACCGCCGCTCAGAACCTGAAAAACAAGACGAGCAACCAAGAGGCTAGAACCATCATCA |
| | | CCGACAGAATCGAGAGAATTCAGAATCAGTGGGACGAGGTGCAAGAGCACCTGCAGAACAGAAG |
| | | ACAGCAACTAAACGAAATGCTCAAGGACAGCACACAGTGGCTGGAGGCCAAGGAGGAGGCGGAG |
| | | CAAGTGTTGGGCCAAGCTAGAGCCAAGCTGGAGAGCTGGAAGGAGGGACCCTACACCGTGGACG |
| | | CCATTCAGAAGAAGATCACCGAAACCAAGCAGCTGGCCAAGGACCTGCGACAGTGGCAGACCAA |
| | | CGTGGACGTGGCCAACGATCTTGCCCTGAAGCTGCTGCGCGACTATAGCGCCGACGACACAAGAA |
| | | AGGTGCACATGATCACCGAGAACATCAACGCTAGCTGGCGTAGCATCCACAAGAGAGTGAGCGAG |
| | | AGAGAGGCGGCTTTGGAGGAAACCCATAGACTGCTGCAGCAGTTTCCCCTGGACCTGGAGAAGTT |
| | | CCTGGCCTGGCTGACCGAAGCCGAAACAACCGCCAACGTGCTGCAAGACGCCACAAGAAAGGAG |
| | | AGACTGCTGGAGGACAGCAAGGGCGTGAAGGAGCTGATGAAGCAGTGGCAAGACCTGCAAGGCG |
| | | AGATCGAAGCGCACACCGACGTGTACCACAACCTGGACGAGAACTCTCAGAAGATCCTGAGAAGC |
| | | CTGGAGGGCAGCGACGACGCCGTGCTGTTACAGAGAAGGCTGGACAACATGAACTTCAAGTGGAG |
| | | CGAGCTGAGAAAGAAGAGCCTGAACATCAGAAGCCACCTGGAGGCTAGCAGCGATCAGTGGAAG |
| | | AGACTGCACCTGAGCCTGCAAGAGCTGCTGGTCTGGCTTCAGTTGAAGGACGATGAGCTGAGCAG |
| | | ACAAGCCCCCATCGGCGGCGACTTCCCCGCCGTGCAGAAGCAGAACGACGTGCATAGAGCCTTCA |
| | | AGAGAGAGCTGAAGACCAAGGAGCCCGTGATCATGAGCACCCTGGAAACGGTGAGAATCTTCCTG |
| | | ACCGAGCAGCCCCTGGAGGGCCTGGAGAAGCTGTACCAAGAGCCTAGAGAGCTGCCCCCCGAGG |
| | | AGAGAGCTCAGAACGTGACAAGACTGCTGAGAAAGCAAGCCGAAGAGGTGAACACCGAGTGGGA |
| | | GAAGCTGAACCTGCACAGCGCCGACTGGCAGAGAAAGATCGACGAAACCCTGGAGAGACTGAGA |
| | | GAATTGCAAGAGGCGACCGACGAGCTGGACCTGAAGCTGAGACAAGCCGAGGTCATCAAGGGCA |
| | | GCTGGCAGCCCGTGGGCGATCTATTAATTGACTCTCTACAAGACCACCTGGAGAAAGTGAAGGCCC |
| | | TGAGAGGCGAGATCGCCCCCCTGAAGGAGAACGTGAGCCACGTGAATGATCTGGCACGTCAGCTG |
| | | ACCACCCTGGGCATACAACTGTCCCCCTATAACCTGAGCACACTGGAGGATTTGAATACTAGATGGA |
| | | AGCTGCTGCAAGTGGCCGTGGAGGACAGAGTAAGACAATTACATGAGGCCCACAGAGACTTCGGC |
| | | CCCGCTTCTCAGCACTTCCTGAGCACAAGCGTGCAAGGCCCCTGGGAGAGAGCCATTTCCCCCAAC |
| | | AAGGTCCCCTACTACATCAACCACGAGACACAGACCACCTGCTGGGACCACCCCAAGATGACCGA |
| | | GCTGTATCAGAGCCTCGCCGATCTGAACAATGTGAGATTCAGCGCCTACAGAACCGCCATGAAGCT |
| | | GAGAAGACTGCAGAAGGCCCTGTGCCTGGACTTATTAAGCCTGAGCGCCGCCTGCGACGCCCTGG |
| | | ATCAGCACAACCTGAAGCAAAACGACCAACCCATGGACATCCTGCAGATCATCAACTGCCTCACCA |
| | | CCATATACGACAGACTGGAACAAGAGCACAACAACCTGGTGAACGTGCCCCTGTGCGTGGACATG |
| | | TGCCTGAACTGGCTGCTGAACGTGTACGACACCGGCAGAACCGGCAGAATCAGAGTGCTGAGCTT |
| | | CAAGACCGGCATCATCAGCCTGTGCAAGGCCCACCTGGAGGACAAATACAGATACCTTTTCAAACA |
| | | AGTGGCTAGCAGCACCGGCTTCTGCGACCAAAGAAGACTGGGTCTGCTGCTGCACGACAGCATTC |
| | | AGATCCCTAGACAGCTGGGCGAGGTGGCTAGCTTCGGCGGCAGCAACATCGAGCCTAGCGTGAGA |
| | | AGCTGCTTTCAGTTCGCCAACAACAAGCCCGAGATAGAGGCAGCCCTCTTCTTAGACTGGATGAGA |
| | | CTGGAGCCTCAGAGCATGGTATGGCTGCCGGTCCTGCACAGAGTGGCCGCCGCCGAAACCGCCAA |
| | | GCACCAAGCCAAGTGCAACATCTGCAAGGAGTGCCCCATCATCGGCTTCAGATACAGAAGCCTGAA |
| | | GCACTTCAACTACGACATCTGTCAGAGCTGCTTCTTCAGCGGCAGAGTGGCCAAGGGCCACAAGAT |
| | | GCACTACCCCATGGTGGAGTACTGCACCCCCACCACAAGCGGCGAGGACGTGAGAGACTTCGCCA |
| | | AGGTGTTGAAGAATAAGTTTAGAACCAAGAGATACTTCGCCAAGCACCCTAGAATGGGCTACCTGC |
| | | CCGTGCAGACCGTGCTGGAGGGCGACAACATGGAAACCCCCGTGACCCTGATCAACTTCTGGCCC |
| | | GTGGACAGCGCCCCCGCTAGCAGCCCTCAGCTGAGCCACGACGACACCCACAGCAGAATCGAGCA |
| | | CTACGCTAGCAGACTGGCCGAGATGGAGAACAGCAACGGCAGCTACCTGAACGACAGCATCAGCC |
| | | CGAATGAGAGCATCGACGACGAGCACCTGCTGATTCAGCACTACTGTCAGAGCCTGAACCAAGAC |
| | | AGCCCCCTGTCTCAGCCTAGAAGCCCCGCTCAGATCCTGATCAGCCTGGAGAGCGAGGAGAGAGG |
| | | CGAGCTGGAGAGAATCCTAGCAGATCTGGAGGAAGAGAACAGAAACCTGCAAGCCGAGTATGACC |
| | | GACTGAAACAGCAACATGAGCACAAAGGCCTGAGCCCACTGCCCTCCCCCCCCGAGATGATGCCC |
| | | ACAAGCCCTCAGAGCCCTAGAGACGCCGAGCTGATCGCTGAAGCGAAGTTACTTAGACAACACAA |
| | | GGGCAGACTGGAGGCTAGAATGCAGATCCTGGAGGACCACAACAAGCAGCTGGAGTCTCAGCTGC |
| | | ACAGACTGAGGCAGCTGCTGGAACAGCCCCAAGCCGAGGCCAAAGTGAACGGCACCACCGTGAG |
| | | CAGTCCTAGCACAAGCCTGCAGAGAAGCGACAGCTCTCAGCCCATGCTGCTGAGAGTGGTGGGCT |
| | | CTCAGACAAGCGACAGCATGGGCGAGGAGGATCTGCTGAGTCCGCCCCAAGACACAAGCACCGGC |
| | | CTGGAAGAGGTGATGGAGCAACTGAACAATAGCTTCCCTAGCAGCAGAGGCAGAAACACCCCCGG |
| | | CAAGCCCATGAGAGAGGACACCATGTGATGACTCGAGAATCAACCTCTGGATTACAAAATTTGTGA |
| | | AAGATTGACTGGTATTCTTAACTATGTTGCTCCTTTTACGCTATGTGGATACGCTGCTTTAATGCCTTT |
| | | GTATCATGCTATTGCTTCCCGTATGGCTTTCATTTTCTCCTCCTTGTATAAATCCTGGTTGCTGTCTCTT |
| | | TATGAGGAGTTGTGGCCCGTTGTCAGGCAACGTGGCGTGGTGTGCACTGTGTTTGCTGACGCAACC |
| | | CCCACTGGTTGGGGCATTGCCACCACCTGTCAGCTCCTTTCCGGGACTTTCGCTTTCCCCCTCCCTA |
| | | TTGCCACGGCGGAACTCATCGCCGCCTGCCTTGCCCGCTGCTGGACAGGGGCTCGGCTGTTGGGCA |
| | | CTGACAATTCCGTGGGGTGTGCCTTCTAGTTGCCAGCCATCTGTTGTTTGCCCCTCCCCCGTGCCTT |
| | | CCTTGACCCTGGAAGGTGCCACTCCCACTGTCCTTTCCTAATAAAATGAGGAAATTGCATCGCATTG |
| | | TCTGAGTAGGTGTCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTACTGAGCTGTGCGA |
| | | TCCCTGCTGGGGACTTTCCGCTGGGGACTTTCCGCTGGGGACTTTCCGCCTTCAGCTAAGGAAGCT |
| | | ACCAATATTTAGAGGTACATTTTGTTCTAGAACAAAATGTACCGGTACATTTTGTTCTGGTACATTTT |
| | | GTTCT |
| 7. | hBGi-DMD- | CTAGAAGCTGCATGTCTAAGCTAGACCCTTCAGATTAAAAATAACTGAGGTAAGGGCCTGGGTAGG |
| | MHCK7- | GGAGGTGGTGTGAGACGCTCCTGTCTCTCCTCTATCTGCCCATCGGCCCTTTGGGGAGGAGGAATG |
| | 4-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTT |
| | | TGGTGGGAAGAAGTCGAGGACTGCTACGAGCGCGAGGACGTGCAGAAGAAAACCTTCACCAAAT |
| | | GGGTCAACGCCCAGTTCAGCAAGTTCGGCAAGCAGCACATCGAGAACCTGTTCAGCGACCTGCAG |
| | | GATGGCAGAAGGCTGCTGGATCTGCTGGAAGGCCTGACAGGACAGAAGCTGCCCAAAGAGAAGG |
| | | GCAGCACAAGAGTGCACGCCCTGAACAACGTGAACAAGGCCCTGAGAGTGCTGCAGAACAACAA |
| | | CGTGGACCTGGTCAACATCGGCAGCACCGACATCGTGGACGGCAACCACAAACTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATTCTGCTGAGCTGGGTCCGACAGAGCACCCGGAATTACCCTCAA |
| | | GTGAACGTGATCAACTTCACCACCTCTTGGAGCGACGGACTGGCCCTGAATGCCCTGATCCACAGC |
| | | CACAGACCTGACCTGTTCGACTGGAACAGCGTCGTGTGTCAGCAGAGCGCCACACAGAGGCTGGA |
| | | ACACGCCTTCAATATCGCCAGATACCAGCTGGGCATCGAGAAACTGCTGGACCCCGAGGATGTGGA |
| | | CACCACCTATCCTGACAAGAAATCCATCCTCATGTACATCACCAGCCTGTTCCAGGTGCTGCCCCAG |
| | | CAGGTTTCCATCGAGGCCATTCAAGAGGTCGAGATGCTGCCCAGACCTCCTAAAGTGACCAAAGAG |
| | | GAACACTTCCAGCTGCACCACCAGATGCACTACTCTCAGCAGATCACCGTGTCTCTGGCCCAGGGC |
| | | TACGAGAGAACAAGCAGCCCCAAGCCTCGGTTCAAGAGCTACGCCTATACACAGGCCGCCTACGTG |
| | | ACCACCAGCGATCCTACAAGAAGCCCATTTCCTAGCCAGCATCTGGAAGCCCCTGAGGACAAGAGC |
| | | TTTGGCAGCAGCCTGATGGAAAGCGAAGTGAACCTGGACCGCTACCAGACAGCCCTGGAAGAGGT |
| | | TCTGAGCTGGCTGCTGTCTGCCGAGGATACACTGCAGGCTCAGGGCGAGATCAGCAACGACGTGG |
| | | AAGTGGTCAAGGACCAGTTTCACACCCACGAGGGCTACATGATGGACCTGACAGCCCACCAGGGC |
| | | AGAGTGGGCAATATTCTGCAGCTGGGCTCCAAGCTGATCGGCACAGGCAAGCTGAGCGAGGACGA |
| | | AGAGACAGAGGTGCAAGAGCAGATGAACCTGCTGAACAGCAGATGGGAGTGTCTGAGAGTGGCC |
| | | AGCATGGAAAAGCAGAGCAACCTGCACCGGGTGCTGATGGATCTCCAGAACCAGAAGCTGAAAGA |
| | | GCTGAACGACTGGCTGACCAAGACCGAGGAACGGACCCGGAAGATGGAAGAGGAACCTCTGGGA |
| | | CCCGACCTGGAAGATCTGAAAAGACAGGTGCAGCAGCATAAGGTGCTGCAAGAGGACCTCGAACA |
| | | AGAGCAAGTGCGCGTGAACAGCCTGACACACATGGTGGTGGTCGTGGATGAGAGCAGCGGAGATC |
| | | ATGCCACAGCCGCTCTGGAAGAACAGCTGAAGGTGCTGGGAGACAGATGGGCCAATATCTGCCGG |
| | | TGGACCGAGGATAGATGGGTGCTGCTCCAGGACATCCTGCTGAAGTGGCAGCGGCTGACAGAGGA |
| | | ACAGTGCCTGTTTAGCGCCTGGCTGTCCGAGAAAGAGGACGCCGTCAACAAGATCCACACCACCG |
| | | GCTTCAAGGATCAGAATGAGATGCTGAGCAGCCTGCAGAAACTGGCCGTGCTGAAGGCTGACCTG |
| | | GAAAAGAAAAAGCAGTCCATGGGCAAGCTGTACTCCCTGAAGCAGGACCTGCTGAGCACACTGAA |
| | | GAACAAGTCTGTGACCCAGAAAACCGAGGCCTGGCTGGACAACTTCGCCCGGTGTTGGGATAACC |
| | | TGGTGCAGAAGCTGGAAAAGTCCACCGCTCAGATCTCTCAGGCCGTGACCACAACACAGCCTTCT |
| | | CTGACCCAGACCACCGTGATGGAAACAGTGACCACAGTGACAACCCGCGAGCAGATCCTGGTCAA |
| | | GCACGCCCAAGAAGAACTGCCTCCTCCACCTCCTCAGAAGAAACGGCAGATCACAGTGGACAGCG |
| | | AGATCCGGAAGAGACTGGACGTGGACATCACCGAGCTGCACAGCTGGATCACCAGATCCGAAGCC |
| | | GTGCTGCAGTCCCCTGAGTTCGCCATCTTCAGAAAAGAGGGCAACTTCTCCGACCTGAAAGAGAA |
| | | AGTCAACGCCATCGAGAGAGAGAAGGCCGAGAAGTTCCGGAAGCTGCAGGACGCCTCTAGATCTG |
| | | CCCAGGCTCTGGTGGAACAGATGGTCAACGAAGGCGTGAACGCCGACAGCATCAAGCAGGCCTCA |
| | | GAGCAGCTGAACTCCCGGTGGATCGAGTTCTGTCAGCTCCTGAGCGAGAGACTGAACTGGCTGGA |
| | | ATACCAGAACAATATCATTGCCTTCTACAACCAGCTCCAGCAGCTCGAACAGATGACCACCACAGC |
| | | CGAGAATTGGCTGAAGATCCAGCCTACCACACCTAGCGAGCCCACCGCCATTAAGAGCCAGCTGAA |
| | | AATCTGCAAGGACGAAGTGAATCGGCTGAGCGATCTGCAGCCCCAGATCGAAAGACTGAAAATCC |
| | | AGTCTATCGCCCTCAAAGAGAAAGGACAGGGCCCCATGTTCCTGGACGCCGATTTTGTGGCCTTTA |
| | | CCAACCACTTCAAACAGGTGTTCTCCGACGTGCAGGCCCGGGAAAAAGAGCTGCAGACCATCTTC |
| | | GACACCCTGCCTCCAATGAGATACCAAGAGACAATGAGCGCCATCCGGACCTGGGTGCAGCAAAG |
| | | CGAGACAAAGCTGAGCATCCCACAGCTGAGCGTGACCGACTACGAGATCATGGAACAGAGACTGG |
| | | GCGAACTGCAGGCCCTCCAGTCTAGCCTGCAAGAACAGCAGTCCGGCCTGTACTACCTGAGCACA |
| | | ACCGTGAAAGAGATGAGCAAGAAGGCCCCTAGCGAGATCTCCCGGAAGTACCAGAGCGAGTTCGA |
| | | AGAGATCGAAGGCCGGTGGAAGAAGCTGTCTAGCCAGCTGGTTGAGCACTGCCAGAAACTCGAAG |
| | | AACAAATGAACAAGCTGCGCAAGATCCAGAATCACATCCAGACGCTGAAGAAATGGATGGCCGAG |
| | | GTGGACGTGTTCCTGAAAGAAGAGTGGCCCGCTCTGGGCGACTCCGAGATTCTGAAGAAACAGCT |
| | | CAAACAGTGCCGGCTGCTGGTGTCCGATATCCAGACAATCCAGCCAAGCCTGAACTCCGTGAATGA |
| | | AGGCGGCCAGAAGATCAAGAACGAGGCCGAGCCTGAGTTTGCCAGCAGACTGGAAACCGAACTG |
| | | AAAGAACTCAACACCCAGTGGGACCACATGTGCCAGCAAGTGTACGCCCGGAAAGAGGCCCTGAA |
| | | AGGCGGACTCGAAAAGACTGTGTCTCTGCAGAAAGACCTGTCTGAGATGCACGAGTGGATGACCC |
| | | AGGCCGAAGAGGAATACCTGGAACGGGACTTCGAGTACAAGACCCCTGATGAGCTGCAAAAAGCC |
| | | GTCGAGGAAATGAAGCGGGCCAAAGAAGAGGCCCAGCAGAAAGAAGCCAAAGTCAAGCTGCTGA |
| | | CCGAGTCCGTGAATAGCGTTATCGCTCAGGCCCCTCCTGTGGCTCAAGAGGCTCTCAAGAAAGAAC |
| | | TGGAAACTCTGACCACCAACTACCAGTGGCTGTGCACCAGACTGAACGGCAAGTGCAAGACACTG |
| | | GAAGAAGTGTGGGCCTGCTGGCACGAACTGCTGTCCTATCTGGAAAAGGCCAACAAGTGGCTGAA |
| | | CGAGGTGGAATTCAAGCTGAAAACCACCGAGAACATCCCTGGCGGCGCTGAAGAGATTAGCGAGG |
| | | TGCTGGACAGCCTGGAAAACCTGATGAGACACAGCGAGGACAACCCCAATCAGATCAGAATCCTG |
| | | GCTCAGACCCTGACCGATGGCGGCGTGATGGACGAGCTGATCAACGAGGAACTCGAAACCTTCAA |
| | | CAGCCGGTGGCGGGAACTGCACGAGGAAGCTGTTCGGAGGCAGAAGTTGCTGGAACAGTCTATCC |
| | | AGAGCGCACAAGAAACCGAGAAGTCCCTGCACCTGATCCAAGAGAGCCTGACCTTCATCGACAAG |
| | | CAGCTGGCCGCTTATATCGCCGACAAGGTGGACGCTGCTCAGATGCCCCAAGAGGCACAGAAGATT |
| | | CAGAGCGACCTGACCAGCCACGAGATTAGCCTGGAAGAAATGAAGAAGCACAACCAGGGCAAAG |
| | | AGGCCGCTCAGAGAGTCCTGAGCCAGATCGATGTGGCACAGAAAAAGCTCCAGGATGTGTCCATG |
| | | AAGTTTCGGCTGTTTCAGAAGCCCGCCAACTTCGAGCAGAGACTGCAAGAGTCCAAGATGATCCT |
| | | GGACGAAGTCAAAATGCATCTGCCCGCACTGGAAACAAAGTCCGTGGAACAAGAAGTGGTGCAGT |
| | | CCCAGCTGAACCACTGCGTGAACCTGTACAAGAGCCTGTCCGAAGTGAAGTCTGAGGTGGAAATG |
| | | GTCATCAAGACCGGCAGGCAGATCGTCCAGAAGAAGCAGACCGAGAATCCCAAAGAACTCGACG |
| | | AGAGAGTGACCGCTCTGAAGCTGCACTACAATGAGCTGGGCGCCAAAGTGACCGAGCGGAAGCA |
| | | ACAGCTTGAGAAGTGCCTGAAACTGTCTCGGAAGATGCGGAAAGAAATGAACGTGCTGACAGAGT |
| | | GGCTGGCCGCCACCGATATGGAACTGACTAAGAGAAGCGCCGTGGAAGGCATGCCCAGCAACCTG |
| | | GATAGTGAAGTGGCCTGGGGCAAAGCCACTCAGAAAGAGATTGAGAAGCAGAAGGTCCACCTGA |
| | | AGTCCATCACCGAAGTGGGCGAAGCCCTGAAAACCGTGCTGGGAAAGAAAGAGACACTGGTCGA |
| | | GGACAAGCTGTCCCTGCTGAATTCCAACTGGATCGCCGTGACCTCCAGAGCTGAGGAATGGCTGAA |
| | | TCTGCTGCTTGAGTACCAGAAACACATGGAAACGTTCGACCAGAACGTCGACCACATCACAAAGT |
| | | GGATCATCCAGGCTGATACCCTGCTGGACGAGTCCGAGAAGAAGAAACCCCAACAAAAAGAAGAT |
| | | GTGCTGAAGCGGCTGAAAGCCGAGCTGAATGACATCCGGCCTAAGGTGGACAGCACCAGAGATCA |
| | | GGCAGCCAACCTGATGGCCAACAGAGGCGACCACTGTCGGAAGCTCGTGGAACCTCAGATCAGCG |
| | | AGCTTAACCACAGATTTGCCGCCATCAGCCACCGGATCAAGACAGGCAAGGCCAGCATTCCTCTCA |
| | | AAGAGCTTGAGCAGTTCAACAGCGACATCCAAAAGCTGCTCGAACCCCTGGAAGCCGAGATCCAA |
| | | CAGGGCGTCAACCTCAAAGAAGAAGATTTCAACAAGGACATGAACGAGGACAATGAGGGCACCG |
| | | TCAAAGAACTGCTGCAGCGGGGCGATAATCTGCAGCAGCGGATTACCGATGAGCGCAAGCGGGAA |
| | | GAGATCAAGATTAAGCAGCAGCTGCTGCAGACCAAGCACAACGCCCTGAAGGACCTGCGGAGCCA |
| | | GAGAAGAAAGAAGGCCCTGGAAATCAGCCACCAGTGGTATCAGTACAAGCGGCAGGCCGACGAC |
| | | CTGCTGAAGTGCCTGGATGACATCGAGAAGAAGCTGGCCTCTCTGCCCGAGCCTAGGGACGAGAG |
| | | AAAGATCAAAGAGATCGACCGCGAGCTGCAGAAAAAGAAAGAGGAACTGAACGCCGTCCGCAGA |
| | | CAGGCCGAAGGACTTTCTGAAGATGGCGCCGCTATGGCCGTGGAACCCACACAGATTCAGCTGAG |
| | | CAAGCGGTGGCGGGAAATCGAGAGCAAGTTCGCCCAGTTCCGGCGGCTGAATTTCGCCCAGATCC |
| | | ACACCGTGCGGGAAGAGACAATGATGGTCATGACAGAGGACATGCCCCTTGAGATCAGCTACGTGC |
| | | CCAGCACCTACCTGACCGAGATCACCCATGTGTCTCAGGCCCTGCTGGAAGTGGAACAGCTGCTGA |
| | | ATGCCCCTGACCTGTGCGCCAAGGACTTCGAGGACCTGTTCAAGCAAGAGGAAAGCCTGAAGAAC |
| | | ATCAAGGACAGCCTGCAGCAGAGCAGCGGCCGGATCGATATCATCCACAGCAAGAAAACCGCCGC |
| | | ACTGCAGAGCGCCACACCTGTGGAAAGAGTGAAGCTGCAAGAGGCCCTGAGCCAGCTGGATTTCC |
| | | AGTGGGAGAAAGTGAACAAGATGTACAAGGACCGGCAGGGCAGATTCGACCGCAGCGTTGAAAA |
| | | GTGGCGGCGGTTCCACTACGACATCAAGATCTTCAACCAGTGGCTGACCGAGGCCGAGCAGTTCCT |
| | | GAGAAAGACACAGATCCCCGAGAACTGGGAGCACGCCAAGTACAAGTGGTATCTGAAAGAGCTGC |
| | | AGGACGGCATCGGCCAGAGACAGACAGTCGTGCGGACACTGAATGCCACCGGCGAGGAAATCATC |
| | | CAGCAGTCCAGCAAGACCGACGCCAGCATCCTGCAAGAGAAGCTGGGCAGCCTGAACCTGCGGTG |
| | | GCAAGAAGTGTGCAAGCAGCTGAGCGACCGGAAGAAGCGGCTGGAAGAACAGAAGAATATCCTG |
| | | AGCGAGTTCCAGCGGGACCTGAACGAGTTCGTGCTGTGGCTCGAAGAGGCCGACAATATCGCCAG |
| | | CATTCCCCTGGAACCTGGCAAAGAGCAGCAGCTGAAAGAAAAGCTGGAACAAGTGAAACTGCTG |
| | | GTCGAGGAACTGCCCCTGAGACAGGGAATCCTGAAACAGCTGAACGAGACAGGCGGCCCTGTGCT |
| | | GGTGTCTGCCCCTATTTCTCCCGAGGAACAGGACAAGCTGGAAAACAAGCTGAAGCAGACCAACC |
| | | TGCAGTGGATCAAGGTGTCCAGAGCACTGCCTGAGAAGCAGGGCGAGATCGAGGCCCAGATCAAG |
| | | GACCTGGGACAGCTCGAAAAGAAGCTTGAGGACCTCGAAGAACAGCTCAACCATCTGCTGCTTTG |
| | | GCTGAGCCCCATCCGGAACCAGCTGGAAATCTACAACCAGCCTAATCAAGAGGGCCCCTTCGACGT |
| | | GAAAGAAACCGAGATTGCCGTGCAGGCCAAGCAGCCCGATGTGGAAGAGATCCTGTCTAAGGGCC |
| | | AGCACCTGTACAAAGAGAAGCCCGCCACACAGCCCGTGAAGCGGAAACTGGAAGATCTGAGCAG |
| | | CGAGTGGAAGGCCGTGAACCGGCTGCTGCAAGAACTGAGAGCCAAACAGCCTGATCTGGCCCCTG |
| | | GCCTGACAACAATTGGCGCCTCTCCTACACAGACCGTGACACTGGTCACACAGCCTGTGGTCACCA |
| | | AAGAGACAGCCATCAGCAAACTGGAAATGCCCAGCAGTCTGATGCTCGAAGTGCCCGCACTGGCC |
| | | GACTTCAATAGAGCCTGGACCGAGCTGACCGACTGGCTCAGTCTGCTGGACCAAGTCATCAAGTCC |
| | | CAGAGAGTGATGGTCGGAGATCTTGAGGACATCAACGAGATGATCATTAAGCAGAAAGCCACCATG |
| | | CAGGACCTTGAGCAGAGAAGGCCCCAGCTTGAAGAACTGATCACCGCCGCTCAGAACCTGAAAAA |
| | | CAAGACCAGCAATCAAGAAGCCCGGACCATCATCACCGACCGGATCGAGAGAATCCAGAACCAGT |
| | | GGGACGAAGTGCAAGAGCATCTGCAGAACAGACGGCAGCAGCTCAATGAGATGCTGAAGGACAG |
| | | CACACAGTGGCTGGAAGCCAAAGAAGAAGCCGAGCAGGTTCTCGGACAGGCCAGAGCTAAGCTG |
| | | GAATCTTGGAAAGAGGGACCCTACACCGTCGACGCCATCCAGAAGAAGATCACCGAGACAAAGCA |
| | | GCTGGCCAAGGATCTGAGACAGTGGCAGACCAATGTGGACGTGGCCAACGACCTGGCTCTGAAGC |
| | | TGCTGAGAGACTACAGCGCCGACGACACCAGAAAGGTGCACATGATCACCGAAAACATCAACGCC |
| | | TCTTGGCGGAGCATCCACAAGCGGGTGTCAGAGAGAGAAGCCGCTCTGGAAGAAACACATAGACT |
| | | GCTCCAGCAGTTCCCACTCGACCTGGAAAAGTTCCTGGCCTGGCTGACAGAAGCCGAAACCACCG |
| | | CTAACGTGCTCCAGGATGCCACCAGAAAAGAGCGGCTGTTGGAGGACAGCAAGGGCGTCAAAGA |
| | | ACTCATGAAGCAGTGGCAGGATCTGCAAGGGGAGATTGAAGCCCACACCGACGTGTACCACAACC |
| | | TGGACGAGAACAGCCAGAAGATCCTGCGGTCCCTGGAAGGCTCTGATGATGCTGTGCTGCTTCAGA |
| | | GGCGGCTGGACAACATGAACTTCAAGTGGAGCGAGCTGCGGAAGAAGTCCCTGAACATCAGAAGC |
| | | CACCTGGAAGCCTCCAGCGACCAGTGGAAAAGACTGCACCTGTCTCTCCAAGAGCTGCTCGTGTG |
| | | GCTGCAGCTGAAGGATGACGAGCTGAGTAGACAGGCCCCTATCGGCGGAGATTTTCCCGCCGTGCA |
| | | GAAACAGAACGACGTGCACCGGGCCTTCAAGAGAGAGCTGAAAACAAAAGAACCCGTCATCATG |
| | | AGCACCCTGGAAACCGTGCGGATCTTTCTGACCGAGCAGCCTCTGGAAGGACTGGAAAAGCTGTA |
| | | CCAAGAGCCTAGAGAGCTGCCTCCTGAAGAAAGGGCCCAGAACGTGACACGGCTGCTTAGAAAGC |
| | | AGGCCGAGGAAGTGAACACCGAATGGGAGAAGCTGAACCTCCACTCCGCCGACTGGCAGCGGAA |
| | | GATCGATGAGACACTGGAACGGCTGCGGGAACTGCAAGAAGCCACAGACGAGCTGGACCTGAAA |
| | | CTGCGGCAGGCTGAAGTGATCAAAGGCTCCTGGCAGCCAGTGGGCGATCTGCTGATCGATAGCCTG |
| | | CAGGACCATCTGGAAAAAGTCAAGGCCCTGCGGGGAGAGATCGCCCCTCTCAAAGAAAACGTGTC |
| | | CCACGTGAACGATCTGGCCAGGCAGCTGACAACACTGGGCATCCAGCTGAGCCCTTACAACCTGTC |
| | | TACCTTGGAGGACCTGAATACCCGGTGGAAGCTGCTCCAAGTGGCCGTCGAGGATAGAGTGCGGC |
| | | AGCTGCATGAGGCTCACAGAGATTTTGGACCCGCCAGCCAGCACTTCCTGAGCACATCTGTTCAAG |
| | | GCCCCTGGGAGAGAGCTATCAGCCCTAACAAGGTGCCCTACTACATCAACCACGAGACACAGACC |
| | | ACCTGTTGGGATCACCCCAAGATGACCGAACTGTATCAGTCCCTGGCCGACCTGAACAACGTGCGG |
| | | TTTAGCGCCTACCGGACCGCCATGAAGCTGCGCAGACTGCAGAAAGCTCTGTGCCTGGACCTGCTG |
| | | TCTCTGAGCGCTGCTTGTGATGCCCTGGACCAGCATAACCTCAAGCAGAACGACCAGCCTATGGAC |
| | | ATCCTGCAGATCATCAACTGCCTGACCACCATCTACGACCGGCTCGAACAAGAGCACAACAACCTG |
| | | GTCAACGTGCCCCTGTGCGTGGACATGTGCCTGAATTGGCTGCTGAACGTGTACGACACCGGCAGA |
| | | ACCGGCAGGATCAGAGTGCTGAGCTTCAAGACCGGCATCATCTCCCTGTGCAAGGCCCACCTTGAG |
| | | GATAAGTACCGCTACCTGTTTAAGCAGGTCGCCAGCAGCACCGGCTTTTGCGACCAAAGAAGGCTG |
| | | GGACTGCTGCTCCACGACAGCATTCAGATCCCTAGACAGCTGGGCGAAGTGGCCTCTTTCGGCGGC |
| | | TCTAATATCGAGCCTAGCGTGCGGAGCTGCTTCCAGTTCGCCAACAACAAGCCCGAGATTGAGGCC |
| | | GCACTGTTCCTGGACTGGATGAGACTGGAACCCCAGAGCATGGTCTGGCTGCCTGTGCTGCATAGA |
| | | GTGGCCGCAGCCGAAACAGCCAAGCACCAGGCCAAGTGCAACATCTGCAAAGAGTGCCCCATCAT |
| | | CGGCTTCCGGTACAGATCCCTGAAGCACTTCAACTACGATATCTGCCAGTCCTGCTTCTTCAGCGGC |
| | | AGAGTGGCCAAGGGCCACAAGATGCACTACCCCATGGTGGAATACTGCACCCCTACCACCTCTGGC |
| | | GAGGACGTGCGGGATTTTGCCAAGGTGCTCAAGAACAAGTTCCGGACCAAGCGCTACTTCGCTAA |
| | | GCACCCCAGAATGGGCTACCTGCCAGTGCAGACAGTGCTTGAGGGCGACAACATGGAAACCCCTG |
| | | TGACTCTGATCAACTTCTGGCCCGTGGATAGCGCCCCTGCTAGTTCTCCTCAGCTGTCTCACGACGA |
| | | TACCCACAGCAGAATCGAGCACTACGCCTCCAGACTGGCCGAGATGGAAAACAGCAACGGCAGCT |
| | | ACCTGAATGACAGCATCAGCCCCAACGAGAGCATCGACGACGAACATCTGCTCATTCAGCACTACT |
| | | GCCAGAGCCTGAATCAGGACAGCCCTCTGTCTCAGCCTAGAAGCCCTGCACAGATCCTGATCAGCC |
| | | TGGAAAGCGAGGAACGCGGCGAGCTGGAAAGAATCCTGGCAGACCTGGAAGAGGAAAACCGGAA |
| | | CCTGCAGGCCGAATACGACAGACTCAAGCAGCAGCACGAGCACAAGGGACTGTCTCCACTGCCTT |
| | | CTCCACCTGAAATGATGCCCACCTCTCCACAGAGCCCCAGAGATGCCGAGCTGATTGCCGAAGCCA |
| | | AACTGCTGAGGCAGCACAAAGGCAGACTCGAAGCCAGAATGCAGATTCTCGAAGATCACAACAAG |
| | | CAGCTCGAATCCCAGCTGCACAGACTTAGGCAGCTGCTGGAACAGCCTCAGGCAGAGGCCAAAGT |
| | | GAATGGCACCACCGTGTCTAGCCCTAGCACAAGCCTGCAGAGATCCGACAGCAGCCAGCCAATGCT |
| | | TCTGAGAGTCGTGGGCTCCCAGACCAGCGATTCTATGGGCGAAGAGGACCTGCTCAGCCCTCCTCA |
| | | GGATACAAGCACCGGCCTGGAAGAAGTGATGGAACAACTGAACAACAGCTTCCCCAGCAGCAGAG |
| | | GCAGAAACACCCCTGGCAAGCCCATGCGCGAGGACACCATGTGATAACTCGAGAATCAACCTCTG |
| | | GATTACAAAATTTGTGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCTCCTCCTTGTATAAA |
| | | TCCTGGTTGCTGTCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCTTTCCGGGACTTT |
| | | CGCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGGGAAGATGT |
| | | CTACTGAGCTGTGCGATCCCTGCTGGGGACTTTCCGCTGGGGACTTTCCGCTGGGGACTTTCCGCCT |
| | | TCAGCTAAGGAAGCTACCAATATTTAGAGGTACATTTTGTTCTAGAACAAAATGTACCGGTACATTTT |
| | | GTTCTGGTACATTTTGTTCT |
| 8. | WPRE3 | AATCAACCTCTGGATTACAAAATTTGTGAAAGATTGACTGGTATTCTTAACTATGTTGCTCCTTTTAC |
| | | GCTATGTGGATACGCTGCTTTAATGCCTTTGTATCATGCTATTGCTTCCCGTATGGCTTTCATTTTCTC |
| | | CTCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGG |
| 9. | Poly A | TGTGCCTTCTAGTTGCCAGCCATCTGTTGTTTGCCCCTCCCCCGTGCCTTCCTTGACCCTGGAAGGT |
| | | GCCACTCCCACTGTCCTTTCCTAATAAAATGAGGAAATTGCATCGCATTGTCTGAGTAGGTGTCATTC |
| | | TATTCTGGGGGGTGGGGTGGGGCAGGACAGCAAGGGGGAGGATTGGGAAGACAATAGCAGGCATG |
| | | CTGGGGATGCGGTGGGCTCTATGG |
| 10. | Post-Tx | AATCAACCTCTGGATTACAAAATTTGTGAAAGATTGACTGGTATTCTTAACTATGTTGCTCCTTTTAC |
| | Reg. | GCTATGTGGATACGCTGCTTTAATGCCTTTGTATCATGCTATTGCTTCCCGTATGGCTTTCATTTTCTC |
| | | CTCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGG |
| 11. | DTS | GAGCTGTGCGATCCCTGCTGGGGACTTTCCGCTGGGGACTTTCCGCTGGGGACTTTCCGCCTTCAG |
| | | CTAAGGAAGCTACCAATATTTAGAGGTACATTTTGTTCTAGAACAAAATGTACCGGTACATTTTGTTC |
| | | TGGTACATTTTGTTCT |
| 12. | Post-Tx | AATCAACCTCTGGATTACAAAATTTGTGAAAGATTGACTGGTATTCTTAACTATGTTGCTCCTTTTAC |
| | Reg. DTS | GCTATGTGGATACGCTGCTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGGGGACTTTCCGCTG |
| | | GGGACTTTCCGCCTTCAGCTAAGGAAGCTACCAATATTTAGAGGTACATTTTGTTCTAGAACAAAAT |
| | | GTACCGGTACATTTTGTTCTGGTACATTTTGTTCT |
| 13. | Dystrophin- | MLWWEEVEDCYEREDVQKKTFTKWVNAQFSKFGKQHIENLFSDLQDGRRLLDLLEGLTGQKLPKEK |
| | WT | TNSEKILLSWVRQSTRNYPQVNVINFTTSWSDGLALNALIHSHRPDLFDWNSVVCQQSATQRLEHAFNI |
| | | GSTRVHALNNVNKALRVLQNNNVDLVNIGSTDIVDGNHKLTLGLIWNIILHWQVKNVMKNIMAGLQQ |
| | | ARYQLGIEKLLDPEDVDTTYPDKKSILMYITSLFQVLPQQVSIEAIQEVEMLPRPPKVTKEEHFQLHHQ |
| | | MHYSQQITVSLAQGYERTSSPKPRFKSYAYTQAAYVTTSDPTRSPFPSQHLEAPEDKSFGSSLMESEVN |
| | | LDRYQTALEEVLSWLLSAEDTLQAQGEISNDVEVVKDQFHTHEGYMMDLTAHQGRVGNILQLGSKLIG |
| | | TGKLSEDEETEVQEQMNLLNSRWECLRVASMEKQSNLHRVLMDLQNQKLKELNDWLTKTEERTRKM |
| | | EEEPLGPDLEDLKRQVQQHKVLQEDLEQEQVRVNSLTHMVVVVDESSGDHATAALEEQLKVLGDRWA |
| | | NICRWTEDRWVLLQDILLKWQRLTEEQCLFSAWLSEKEDAVNKIHTTGFKDQNEMLSSLQKLAVLKAD |
| | | LEKKKQSMGKLYSLKQDLLSTLKNKSVTQKTEAWLDNFARCWDNLVQKLEKSTAQISQAVTTTQPSLT |
| | | QTTVMETVTTVTTREQILVKHAQEELPPPPPQKKRQITVDSEIRKRLDVDITELHSWITRSEAVLQSPEF |
| | | AIFRKEGNFSDLKEKVNAIEREKAEKFRKLQDASRSAQALVEQMVNEGVNADSIKQASEQLNSRWIEFC |
| | | QLLSERLNWLEYQNNIIAFYNQLQQLEQMTTTAENWLKIQPTTPSEPTAIKSQLKICKDEVNRLSDLQP |
| | | QIERLKIQSIALKEKGQGPMFLDADFVAFTNHFKQVFSDVQAREKELQTIFDTLPPMRYQETMSAIRTW |
| | | VQQSETKLSIPQLSVTDYEIMEQRLGELQALQSSLQEQQSGLYYLSTTVKEMSKKAPSEISRKYQSEFEE |
| | | IEGRWKKLSSQLVEHCQKLEEQMNKLRKIQNHIQTLKKWMAEVDVFLKEEWPALGDSEILKKQLKQC |
| | | RLLVSDIQTIQPSLNSVNEGGQKIKNEAEPEFASRLETELKELNTQWDHMCQQVYARKEALKGGLEKT |
| | | VSLQKDLSEMHEWMTQAEEEYLERDFEYKTPDELQKAVEEMKRAKEEAQQKEAKVKLLTESVNSVIA |
| | | QAPPVAQEALKKELETLTTNYQWLCTRLNGKCKTLEEVWACWHELLSYLEKANKWLNEVEFKLKTT |
| | | ENIPGGAEEISEVLDSLENLMRHSEDNPNQIRILAQTLTDGGVMDELINEELETFNSRWRELHEEAVRRQ |
| | | KLLEQSIQSAQETEKSLHLIQESLTFIDKQLAAYIADKVDAAQMPQEAQKIQSDLTSHEISLEEMKKHNQ |
| | | GKEAAQRVLSQIDVAQKKLQDVSMKFRLFQKPANFEQRLQESKMILDEVKMHLPALETKSVEQEVVQ |
| | | SQLNHCVNLYKSLSEVKSEVEMVIKTGRQIVQKKQTENPKELDERVTALKLHYNELGAKVTERKQQLE |
| | | KCLKLSRKMRKEMNVLTEWLAATDMELTKRSAVEGMPSNLDSEVAWGKATQKEIEKQKVHLKSITEV |
| | | GEALKTVLGKKETLVEDKLSLLNSNWIAVTSRAEEWLNLLLEYQKHMETFDQNVDHITKWIIQADTLL |
| | | DESEKKKPQQKEDVLKRLKAELNDIRPKVDSTRDQAANLMANRGDHCRKLVEPQISELNHRFAAISHR |
| | | IKTGKASIPLKELEQFNSDIQKLLEPLEAEIQQGVNLKEEDFNKDMNEDNEGTVKELLQRGDNLQQRIT |
| | | DERKREEIKIKQQLLQTKHNALKDLRSQRRKKALEISHQWYQYKRQADDLLKCLDDIEKKLASLPEPR |
| | | DERKIKEIDRELQKKKEELNAVRRQAEGLSEDGAAMAVEPTQIQLSKRWREIESKFAQFRRLNFAQIHT |
| | | VREETMMVMTEDMPLEISYVPSTYLTEITHVSQALLEVEQLLNAPDLCAKDFEDLFKQEESLKNIKDSL |
| | | QQSSGRIDIIHSKKTAALQSATPVERVKLQEALSQLDFQWEKVNKMYKDRQGRFDRSVEKWRRFHYDI |
| | | KIFNQWLTEAEQFLRKTQIPENWEHAKYKWYLKELQDGIGQRQTVVRTLNATGEEIIQQSSKTDASILQ |
| | | EKLGSLNLRWQEVCKQLSDRKKRLEEQKNILSEFQRDLNEFVLWLEEADNIASIPLEPGKEQQLKEKLE |
| | | QVKLLVEELPLRQGILKQLNETGGPVLVSAPISPEEQDKLENKLKQTNLQWIKVSRALPEKQGEIEAQIK |
| | | DLGQLEKKLEDLEEQLNHLLLWLSPIRNQLEIYNQPNQEGPFDVKETEIAVQAKQPDVEEILSKGQHLY |
| | | KEKPATQPVKRKLEDLSSEWKAVNRLLQELRAKQPDLAPGLTTIGASPTQTVTLVTQPVVTKETAISKLE |
| | | MPSSLMLEVPALADFNRAWTELTDWLSLLDQVIKSQRVMVGDLEDINEMIIKQKATMQDLEQRRPQLE |
| | | ELITAAQNLKNKTSNQEARTIITDRIERIQNQWDEVQEHLQNRRQQLNEMLKDSTQWLEAKEEAEQVL |
| | | GQARAKLESWKEGPYTVDAIQKKITETKQLAKDLRQWQTNVDVANDLALKLLRDYSADDTRKVHMI |
| | | TENINASWRSIHKRVSEREAALEETHRLLQQFPLDLEKFLAWLTEAETTANVLQDATRKERLLEDSKGV |
| | | KELMKQWQDLQGEIEAHTDVYHNLDENSQKILRSLEGSDDAVLLQRRLDNMNFKWSELRKKSLNIRS |
| | | HLEASSDQWKRLHLSLQELLVWLQLKDDELSRQAPIGGDFPAVQKQNDVHRAFKRELKTKEPVIMSTL |
| | | ETVRIFLTEQPLEGLEKLYQEPRELPPEERAQNVTRLLRKQAEEVNTEWEKLNLHSADWQRKIDETLER |
| | | LRELQEATDELDLKLRQAEVIKGSWQPVGDLLIDSLQDHLEKVKALRGEIAPLKENVSHVNDLARQLT |
| | | TLGIQLSPYNLSTLEDLNTRWKLLQVAVEDRVRQLHEAHRDFGPASQHELSTSVQGPWERAISPNKVPY |
| | | YINHETQTTCWDHPKMTELYQSLADLNNVRFSAYRTAMKLRRLQKALCLDLLSLSAACDALDQHNLK |
| | | QNDQPMDILQIINCLTTIYDRLEQEHNNLVNVPLCVDMCLNWLLNVYDTGRTGRIRVLSFKTGIISLCKA |
| | | HLEDKYRYLFKQVASSTGFCDQRRLGLLLHDSIQIPRQLGEVASFGGSNIEPSVRSCFQFANNKPEIEAA |
| | | LFLDWMRLEPQSMVWLPVLHRVAAAETAKHQAKCNICKECPIIGFRYRSLKHFNYDICQSCFFSGRVAK |
| | | GHKMHYPMVEYCTPTTSGEDVRDFAKVLKNKFRTKRYFAKHPRMGYLPVQTVLEGDNMETPVTLINF |
| | | WPVDSAPASSPQLSHDDTHSRIEHYASRLAEMENSNGSYLNDSISPNESIDDEHLLIQHYCQSLNQDSPL |
| | | SQPRSPAQILISLESEERGELERILADLEEENRNLQAEYDRLKQQHEHKGLSPLPSPPEMMPTSPQSPRD |
| | | AELIAEAKLLRQHKGRLEARMQILEDHNKQLESQLHRLRQLLEQPQAEAKVNGTTVSSPSTSLQRSDSSQ |
| | | PMLLRVVGSQTSDSMGEEDLLSPPQDTSTGLEEVMEQLNNSFPSSRGRNTPGKPMREDTM** |
| 14. | NP-CBA- | TCTGGATCCACTGCTTAAATACGGACGAGGACAGGGCCCTGTCTCCTCAGCTTCAGGCACCACCAC |
| | hBGi-DMD- | TGACCTGGGACAGTGAATCGTAAGTACTAGCAGCTACAATCCAGCTACCATTCTGCTTTTATTTTATG |
| | 3-WPRE3- | GTTGGGATAAGGCTGGATTATTCTGAGTCCAAGCTAGGCCCTTTTGCTAATCATGTTCATACCTCTTA |
| | DTS | TCTTCCTCCCACAGCTCCTGGGCAACGTGCTGGTCTGTGTGCTGGCCCATCACTTTGGCAAAGAATT |
| | | GCGATCGCCACCATGCTGTGGTGGGAAGAGGTGGAGGACTGTTACGAGAGAGAGGACGTGCAGAA |
| | | GAAGACATTCACCAAGTGGGTGAACGCTCAGTTCAGCAAGTTCGGCAAGCAGCACATCGAGAACC |
| | | TGTTTAGCGATCTACAAGATGGCAGAAGACTGCTGGACCTGCTGGAGGGCCTGACCGGGCAGAAG |
| | | CTGCCCAAAGAAAAGGGGTCTACAAGAGTGCACGCCCTGAACAACGTGAACAAGGCCCTGAGAG |
| | | TGCTGCAGAACAACAACGTGGACCTGGTGAACATCGGCAGCACCGACATCGTGGACGGCAACCAC |
| | | AAGCTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATCCTGCTGAGCTGGGTGAGACAGAGCA |
| | | CAAGAAACTACCCCCAAGTGAACGTGATCAACTTCACCACAAGCTGGAGCGACGGCCTGGCCCTG |
| | | AACGCCCTGATCCACAGCCACAGACCCGACCTGTTCGACTGGAACAGCGTGGTGTGTCAGCAGAG |
| | | CGCCACACAGAGACTGGAGCACGCCTTCAACATCGCTAGATATCAGCTGGGCATCGAGAAGTTACT |
| | | GGACCCCGAAGATGTGGACACCACCTACCCCGACAAGAAGAGCATCCTGATGTACATCACAAGCCT |
| | | GTTCCAAGTGCTGCCTCAGCAAGTGAGCATCGAGGCCATCCAAGAAGTCGAGATGCTGCCTAGACC |
| | | CCCCAAGGTGACCAAGGAGGAGCACTTTCAGCTGCACCATCAGATGCACTACTCTCAGCAGATCAC |
| | | AGTCAGCTTGGCACAAGGCTACGAGAGAACAAGCAGCCCCAAGCCTAGATTCAAGAGCTACGCCT |
| | | ACACCCAAGCCGCCTACGTGACCACAAGTGACCCCACAAGAAGCCCCTTCCCTTCTCAGCACCTG |
| | | GAGGCCCCCGAGGACAAGAGCTTCGGCAGCAGCCTGATGGAGAGCGAGGTGAACCTGGACAGAT |
| | | ATCAGACCGCGTTAGAAGAAGTGTTGAGCTGGCTGCTGAGCGCCGAGGACACCCTGCAAGCCCAA |
| | | GGCGAGATCAGCAACGATGTGGAGGTGGTGAAGGATCAGTTCCACACCCACGAGGGCTACATGAT |
| | | GGACCTGACCGCCCACCAAGGCAGAGTGGGCAACATCCTGCAGCTGGGCAGCAAGCTGATCGGCA |
| | | CCGGCAAGCTGAGCGAGGACGAGGAAACCGAGGTTCAAGAGCAAATGAACCTGCTAAACAGCAG |
| | | ATGGGAGTGCCTGAGAGTGGCTAGCATGGAAAAGCAGAGTAACCTGCACAGAGTGCTGATGGACC |
| | | TGCAGAATCAGAAATTGAAAGAACTTAATGATTGGCTGACCAAGACCGAGGAGAGAACAAGAAAG |
| | | ATGGAGGAGGAGCCCCTGGGCCCCGACCTGGAGGACCTGAAGAGACAAGTGCAGCAGCACAAGG |
| | | TGCTGCAAGAGGACCTGGAGCAAGAGCAAGTGAGAGTGAACTCTCTGACACACATGGTGGTGGTA |
| | | GTGGACGAGAGCAGCGGCGATCACGCTACCGCGGCGCTGGAAGAGCAGCTGAAAGTGCTGGGCG |
| | | ACAGATGGGCCAACATCTGCAGATGGACCGAGGACAGATGGGTGCTGCTGCAAGACATCCTGCTG |
| | | AAGTGGCAGAGACTGACCGAGGAGCAGTGCCTGTTCAGCGCCTGGCTGAGCGAGAAGGAAGACG |
| | | CTGTGAACAAGATCCACACCACCGGCTTCAAGGATCAGAACGAGATGCTGAGCTCCCTCCAAAAG |
| | | CTCGCAGTGCTGAAGGCCGACCTGGAAAAGAAAAAACAGTCGATGGGCAAGCTGTACAGCCTGAA |
| | | GCAAGACCTGCTGAGCACCCTGAAGAACAAGAGCGTGACACAGAAGACCGAGGCCTGGCTGGAC |
| | | AACTTCGCTAGATGCTGGGACAACCTGGTACAGAAGTTAGAGAAGTCTACCGCTCAGATCAGCCAA |
| | | GCCGTAACCACCACACAACCTAGCCTGACTCAGACCACCGTGATGGAAACCGTGACCACCGTGAC |
| | | AACCCGTGAGCAGATCCTGGTGAAGCACGCCCAAGAGGAGCTGCCCCCCCCCCCCCCTCAGAAGA |
| | | AGAGACAGATCACCGTGGACTCAGAGATTCGCAAGAGACTGGACGTGGACATCACCGAGCTGCAC |
| | | AGCTGGATCACAAGAAGCGAGGCCGTGCTGCAGAGCCCTGAGTTCGCGATCTTTAGAAAGGAGGG |
| | | CAACTTCAGCGACTTGAAGGAGAAAGTGAACGCCATCGAGAGAGAGAAGGCCGAGAAGTTCAGA |
| | | AAGTTGCAAGACGCCTCAAGAAGCGCCCAAGCCCTGGTGGAGCAGATGGTTAACGAAGGCGTGAA |
| | | TGCCGACAGCATCAAGCAAGCTAGCGAGCAGCTGAATAGCCGGTGGATTGAATTCTGTCAGCTGCT |
| | | GAGCGAGAGATTAAACTGGCTGGAGTATCAGAACAACATCATCGCCTTCTACAATCAGCTGCAGCA |
| | | ACTAGAACAAATGACCACCACCGCCGAGAACTGGCTAAAGATTCAGCCCACCACCCCTAGCGAGC |
| | | CCACCGCCATCAAATCTCAGCTGAAAATTTGCAAGGACGAAGTGAACAGACTGTCGGACCTGCAG |
| | | CCTCAAATCGAAAGACTGAAAATTCAGAGCATCGCACTTAAGGAGAAGGGCCAAGGACCCATGTT |
| | | CCTGGACGCCGACTTCGTGGCCTTCACCAACCACTTCAAGCAAGTGTTCAGCGACGTGCAAGCTAG |
| | | AGAGAAGGAGCTGCAGACCATCTTCGACACCCTGCCCCCCATGAGATACCAAGAAACCATGAGCG |
| | | CCATCAGAACCTGGGTGCAGCAGAGCGAAACCAAGCTGAGCATCCCTCAGCTGAGCGTGACCGAC |
| | | TACGAGATCATGGAGCAAAGACTGGGAGAGCTGCAAGCCCTGCAGAGCAGCCTGCAAGAGCAGC |
| | | AGAGCGGCCTGTACTACCTGAGCACCACCGTGAAGGAGATGTCGAAGAAAGCCCCGTCGGAGATC |
| | | AGCAGAAAGTATCAGAGCGAGTTCGAGGAGATCGAGGGCAGATGGAAGAAGCTGAGCAGCCAAC |
| | | TGGTGGAACACTGTCAGAAATTAGAAGAACAGATGAACAAGCTGAGAAAGATTCAGAACCACATT |
| | | CAGACCCTGAAGAAGTGGATGGCCGAGGTGGACGTGTTCCTGAAAGAGGAGTGGCCCGCCCTGGG |
| | | CGATTCCGAAATCTTAAAAAAGCAGCTGAAGCAGTGCAGACTGCTGGTGTCAGATATCCAAACCAT |
| | | TCAACCAAGCCTGAACAGCGTGAACGAAGGCGGGCAGAAGATCAAGAACGAGGCCGAGCCCGAG |
| | | TTCGCTAGCAGACTGGAAACCGAGCTGAAGGAGCTGAATACACAGTGGGACCACATGTGTCAGCA |
| | | AGTGTACGCTAGAAAGGAAGCGCTGAAGGGCGGGCTGGAGAAGACCGTTAGCCTGCAAAAGGATC |
| | | TGAGCGAGATGCACGAGTGGATGACCCAAGCCGAGGAGGAGTACCTGGAGAGAGACTTCGAGTAC |
| | | AAGACCCCCGACGAGCTGCAGAAGGCCGTCGAGGAAATGAAGAGAGCGAAGGAGGAGGCTCAGC |
| | | AGAAGGAAGCCAAGGTGAAGCTGCTGACTGAATCTGTCAACAGTGTGATAGCTCAAGCACCCCCC |
| | | GTTGCCCAAGAGGCACTAAAGAAGGAGCTGGAAACTCTGACCACCAACTATCAGTGGCTGTGCAC |
| | | AAGACTGAACGGCAAGTGCAAGACCCTGGAGGAAGTGTGGGCCTGCTGGCACGAGCTGCTGAGC |
| | | TACCTGGAGAAGGCCAACAAGTGGCTGAACGAGGTGGAGTTCAAGCTGAAGACCACCGAGAACA |
| | | TCCCCGGCGGCGCCGAGGAGATCAGCGAGGTGCTGGACAGCCTGGAGAACCTGATGAGACACAGC |
| | | GAGGACAACCCCAATCAGATCAGAATCCTGGCTCAGACCCTGACCGACGGCGGCGTGATGGACGA |
| | | GCTGATCAACGAGGAGCTGGAAACCTTTAACTCTAGATGGCGAGAACTGCACGAGGAGGCCGTGA |
| | | GAAGACAGAAGCTGCTGGAGCAAAGCATTCAGAGCGCCCAAGAAACCGAGAAGAGCCTGCACCT |
| | | GATCCAAGAGAGCCTGACCTTCATCGACAAGCAGCTGGCCGCCTACATCGCCGACAAGGTGGACG |
| | | CCGCTCAGATGCCACAAGAGGCTCAGAAGATTCAGTCCGATCTGACAAGCCACGAGATCAGCCTG |
| | | GAGGAGATGAAAAAACACAACCAAGGCAAGGAGGCCGCTCAGAGAGTGCTGTCTCAGATCGACG |
| | | TGGCTCAGAAGAAGCTGCAAGACGTGAGCATGAAGTTCAGACTGTTTCAGAAGCCCGCCAACTTC |
| | | GAGCAGCGACTGCAAGAGAGCAAGATGATCCTGGACGAGGTGAAGATGCACCTGCCCGCCCTGGA |
| | | AACCAAGAGCGTGGAGCAAGAGGTGGTGCAGTCTCAGTTGAACCACTGCGTGAACCTGTACAAGA |
| | | GCCTGTCGGAGGTCAAGAGTGAGGTCGAGATGGTGATCAAAACTGGGAGACAGATCGTGCAAAAA |
| | | AAGCAGACCGAGAACCCCAAGGAGCTGGACGAGCGTGTTACCGCCCTGAAGCTGCACTACAACGA |
| | | GCTGGGCGCCAAGGTGACCGAGAGAAAGCAGCAGCTGGAGAAGTGCCTGAAGCTGAGCAGAAAG |
| | | ATGAGAAAGGAGATGAACGTGCTGACCGAGTGGCTGGCCGCCACCGACATGGAGCTGACCAAGAG |
| | | AAGCGCCGTGGAGGGCATGCCTAGCAACCTGGACAGCGAGGTGGCCTGGGGCAAGGCCACACAG |
| | | AAGGAGATCGAGAAGCAGAAGGTGCACCTGAAGAGCATCACCGAGGTGGGCGAGGCCCTGAAGA |
| | | CCGTGCTGGGCAAGAAGGAAACCCTGGTGGAGGACAAGCTGAGCCTGCTGAACTCGAACTGGATC |
| | | GCCGTGACAAGCAGAGCCGAGGAGTGGCTGAACCTGCTGTTAGAGTACCAAAAACACATGGAAAC |
| | | CTTCGATCAGAACGTGGACCACATCACCAAGTGGATCATCCAAGCCGACACCCTGCTGGACGAAA |
| | | GCGAAAAGAAGAAGCCGCAACAAAAGGAGGATGTGCTGAAGAGACTGAAGGCCGAACTGAACG |
| | | ATATCAGACCCAAGGTGGACAGCACACGGGACCAAGCCGCCAACCTGATGGCCAACAGAGGCGAC |
| | | CACTGCAGAAAACTGGTGGAGCCTCAGATCAGCGAGCTGAACCACAGATTCGCCGCCATCAGCCA |
| | | CAGAATCAAAACCGGCAAAGCGAGCATCCCCTTGAAGGAACTGGAGCAATTTAACAGCGACATCC |
| | | AAAAATTGCTCGAACCACTGGAGGCCGAGATTCAGCAAGGCGTGAACCTGAAGGAAGAGGACTTC |
| | | AACAAGGACATGAACGAGGACAACGAGGGCACCGTGAAAGAACTGCTGCAGAGAGGCGACAACC |
| | | TGCAGCAGAGAATCACCGACGAGAGAAAGAGAGAGGAGATCAAGATCAAGCAGCAGCTGCTGCA |
| | | GACTAAGCACAACGCCCTGAAGGATCTGAGATCTCAGAGGAGAAAAAAGGCTCTGGAAATTTCAC |
| | | ATCAGTGGTATCAGTACAAGAGACAAGCAGACGACCTGCTGAAGTGCCTGGACGACATCGAGAAG |
| | | AAGTTAGCTAGCCTGCCCGAGCCTAGAGACGAGAGAAAGATCAAGGAGATCGACAGAGAGTTACA |
| | | AAAGAAGAAGGAGGAGCTGAATGCGGTAAGAAGACAAGCGGAAGGCCTGTCCGAGGACGGCGCA |
| | | GCCATGGCCGTGGAGCCCACACAGATTCAGCTCAGCAAGAGATGGAGAGAGATCGAGAGCAAGTT |
| | | CGCTCAGTTCAGAAGACTGAACTTCGCTCAGATCCACACCGTGAGAGAGGAAACCATGATGGTGAT |
| | | GACCGAGGACATGCCCCTGGAGATCTCATACGTTCCTAGCACCTACCTGACCGAGATCACCCACGT |
| | | GAGCCAAGCCCTGCTGGAGGTGGAGCAGCTGCTGAACGCCCCCGACCTGTGCGCCAAGGACTTCG |
| | | AGGACCTGTTCAAACAAGAGGAGAGCCTGAAGAACATCAAGGACAGCCTGCAGCAAAGTAGCGG |
| | | CAGAATCGACATCATCCACAGCAAGAAGACCGCCGCCCTGCAGAGCGCCACCCCCGTGGAGAGAG |
| | | TGAAGCTTCAAGAGGCCCTTTCGCAGCTGGATTTTCAGTGGGAGAAGGTGAACAAAATGTACAAG |
| | | GACAGACAAGGCAGATTCGACAGAAGCGTGGAGAAGTGGAGAAGATTCCACTACGACATCAAGAT |
| | | CTTCAATCAGTGGCTGACCGAGGCAGAGCAGTTCCTGAGAAAGACACAGATCCCCGAGAACTGGG |
| | | AGCACGCCAAGTACAAGTGGTACCTGAAGGAGCTGCAAGACGGCATCGGGCAGAGACAGACCGT |
| | | GGTGAGAACCCTGAACGCCACCGGCGAGGAGATCATCCAACAAAGCTCCAAAACCGACGCTAGCA |
| | | TCCTGCAAGAGAAACTGGGCAGCCTGAACCTGAGATGGCAAGAGGTGTGCAAGCAGCTGAGCGA |
| | | CCGCAAAAAACGGTTAGAGGAGCAGAAGAACATCCTGAGCGAGTTTCAGCGGGACCTGAACGAG |
| | | TTCGTGCTGTGGCTGGAGGAGGCCGATAACATCGCTAGCATCCCCCTGGAGCCCGGCAAGGAGCAG |
| | | CAGTTAAAAGAAAAACTTGAGCAAGTGAAGCTGCTGGTGGAGGAGCTGCCCCTGAGACAAGGCAT |
| | | CCTGAAGCAGCTGAACGAGACTGGCGGCCCCGTGCTGGTGAGCGCCCCCATCAGCCCCGAGGAGC |
| | | AAGACAAGCTGGAGAACAAGCTGAAGCAGACCAACCTGCAGTGGATCAAGGTGAGCAGAGCCCT |
| | | GCCCGAGAAGCAAGGCGAAATCGAGGCCCAAATAAAGGACCTGGGGCAGCTGGAGAAGAAACTC |
| | | GAAGACTTGGAAGAACAACTCAACCACTTGCTGCTGTGGCTGAGCCCCATCAGAAATCAGCTGGA |
| | | GATCTACAATCAGCCCAACCAAGAGGGGCCATTCGACGTGAAGGAAACCGAGATCGCCGTGCAAG |
| | | CCAAGCAGCCCGATGTGGAGGAGATCCTGAGCAAGGGGCAGCACCTGTACAAGGAGAAGCCCGCC |
| | | ACACAGCCCGTCAAGCGTAAACTAGAGGATCTTTCCTCTGAGTGGAAGGCCGTGAACCGTCTGCTA |
| | | CAAGAGCTGCGGGCTAAGCAACCCGACTTAGCGCCCGGCCTGACCACCATCGGTGCAAGCCCCAC |
| | | GCAGACCGTGACCCTGGTGACCCAACCCGTGGTGACCAAGGAAACCGCCATCAGCAAGCTGGAGA |
| | | TGCCTAGCAGCCTGATGCTGGAGGTGCCCGCCCTGGCCGACTTCAACAGAGCCTGGACCGAGCTG |
| | | ACCGACTGGCTGAGCCTGCTGGACCAAGTGATCAAGTCTCAGAGAGTGATGGTGGGCGATCTGGA |
| | | GGACATCAATGAGATGATCATCAAGCAGAAGGCCACCATGCAAGACCTGGAGCAGAGAAGACCTC |
| | | AGCTGGAGGAGCTGATCACCGCCGCTCAGAACCTGAAAAACAAGACGAGCAACCAAGAGGCTAG |
| | | AACCATCATCACCGACAGAATCGAGAGAATTCAGAATCAGTGGGACGAGGTGCAAGAGCACCTGC |
| | | AGAACAGAAGACAGCAACTAAACGAAATGCTCAAGGACAGCACACAGTGGCTGGAGGCCAAGGA |
| | | GGAGGCGGAGCAAGTGTTGGGCCAAGCTAGAGCCAAGCTGGAGAGCTGGAAGGAGGGACCCTAC |
| | | ACCGTGGACGCCATTCAGAAGAAGATCACCGAAACCAAGCAGCTGGCCAAGGACCTGCGACAGTG |
| | | GCAGACCAACGTGGACGTGGCCAACGATCTTGCCCTGAAGCTGCTGCGCGACTATAGCGCCGACG |
| | | ACACAAGAAAGGTGCACATGATCACCGAGAACATCAACGCTAGCTGGCGTAGCATCCACAAGAGA |
| | | GTGAGCGAGAGAGAGGCGGCTTTGGAGGAAACCCATAGACTGCTGCAGCAGTTTCCCCTGGACCT |
| | | GGAGAAGTTCCTGGCCTGGCTGACCGAAGCCGAAACAACCGCCAACGTGCTGCAAGACGCCACA |
| | | AGAAAGGAGAGACTGCTGGAGGACAGCAAGGGCGTGAAGGAGCTGATGAAGCAGTGGCAAGACC |
| | | TGCAAGGCGAGATCGAAGCGCACACCGACGTGTACCACAACCTGGACGAGAACTCTCAGAAGATC |
| | | CTGAGAAGCCTGGAGGGCAGCGACGACGCCGTGCTGTTACAGAGAAGGCTGGACAACATGAACTT |
| | | CAAGTGGAGCGAGCTGAGAAAGAAGAGCCTGAACATCAGAAGCCACCTGGAGGCTAGCAGCGAT |
| | | CAGTGGAAGAGACTGCACCTGAGCCTGCAAGAGCTGCTGGTCTGGCTTCAGTTGAAGGACGATGA |
| | | GCTGAGCAGACAAGCCCCCATCGGCGGCGACTTCCCCGCCGTGCAGAAGCAGAACGACGTGCATA |
| | | GAGCCTTCAAGAGAGAGCTGAAGACCAAGGAGCCCGTGATCATGAGCACCCTGGAAACGGTGAG |
| | | AATCTTCCTGACCGAGCAGCCCCTGGAGGGCCTGGAGAAGCTGTACCAAGAGCCTAGAGAGCTGC |
| | | CCCCCGAGGAGAGAGCTCAGAACGTGACAAGACTGCTGAGAAAGCAAGCCGAAGAGGTGAACAC |
| | | CGAGTGGGAGAAGCTGAACCTGCACAGCGCCGACTGGCAGAGAAAGATCGACGAAACCCTGGAG |
| | | AGACTGAGAGAATTGCAAGAGGCGACCGACGAGCTGGACCTGAAGCTGAGACAAGCCGAGGTCA |
| | | TCAAGGGCAGCTGGCAGCCCGTGGGCGATCTATTAATTGACTCTCTACAAGACCACCTGGAGAAAG |
| | | TGAAGGCCCTGAGAGGCGAGATCGCCCCCCTGAAGGAGAACGTGAGCCACGTGAATGATCTGGCA |
| | | CGTCAGCTGACCACCCTGGGCATACAACTGTCCCCCTATAACCTGAGCACACTGGAGGATTTGAATA |
| | | CTAGATGGAAGCTGCTGCAAGTGGCCGTGGAGGACAGAGTAAGACAATTACATGAGGCCCACAGA |
| | | GACTTCGGCCCCGCTTCTCAGCACTTCCTGAGCACAAGCGTGCAAGGCCCCTGGGAGAGAGCCAT |
| | | TTCCCCCAACAAGGTCCCCTACTACATCAACCACGAGACACAGACCACCTGCTGGGACCACCCCAA |
| | | GATGACCGAGCTGTATCAGAGCCTCGCCGATCTGAACAATGTGAGATTCAGCGCCTACAGAACCGC |
| | | CATGAAGCTGAGAAGACTGCAGAAGGCCCTGTGCCTGGACTTATTAAGCCTGAGCGCCGCCTGCG |
| | | ACGCCCTGGATCAGCACAACCTGAAGCAAAACGACCAACCCATGGACATCCTGCAGATCATCAACT |
| | | GCCTCACCACCATATACGACAGACTGGAACAAGAGCACAACAACCTGGTGAACGTGCCCCTGTGC |
| | | GTGGACATGTGCCTGAACTGGCTGCTGAACGTGTACGACACCGGCAGAACCGGCAGAATCAGAGT |
| | | GCTGAGCTTCAAGACCGGCATCATCAGCCTGTGCAAGGCCCACCTGGAGGACAAATACAGATACCT |
| | | TTTCAAACAAGTGGCTAGCAGCACCGGCTTCTGCGACCAAAGAAGACTGGGTCTGCTGCTGCACG |
| | | ACAGCATTCAGATCCCTAGACAGCTGGGCGAGGTGGCTAGCTTCGGCGGCAGCAACATCGAGCCTA |
| | | GCGTGAGAAGCTGCTTTCAGTTCGCCAACAACAAGCCCGAGATAGAGGCAGCCCTCTTCTTAGACT |
| | | GGATGAGACTGGAGCCTCAGAGCATGGTATGGCTGCCGGTCCTGCACAGAGTGGCCGCCGCCGAA |
| | | ACCGCCAAGCACCAAGCCAAGTGCAACATCTGCAAGGAGTGCCCCATCATCGGCTTCAGATACAGA |
| | | AGCCTGAAGCACTTCAACTACGACATCTGTCAGAGCTGCTTCTTCAGCGGCAGAGTGGCCAAGGG |
| | | CCACAAGATGCACTACCCCATGGTGGAGTACTGCACCCCCACCACAAGCGGCGAGGACGTGAGAG |
| | | ACTTCGCCAAGGTGTTGAAGAATAAGTTTAGAACCAAGAGATACTTCGCCAAGCACCCTAGAATGG |
| | | GCTACCTGCCCGTGCAGACCGTGCTGGAGGGCGACAACATGGAAACCCCCGTGACCCTGATCAAC |
| | | TTCTGGCCCGTGGACAGCGCCCCCGCTAGCAGCCCTCAGCTGAGCCACGACGACACCCACAGCAG |
| | | AATCGAGCACTACGCTAGCAGACTGGCCGAGATGGAGAACAGCAACGGCAGCTACCTGAACGACA |
| | | GCATCAGCCCGAATGAGAGCATCGACGACGAGCACCTGCTGATTCAGCACTACTGTCAGAGCCTGA |
| | | ACCAAGACAGCCCCCTGTCTCAGCCTAGAAGCCCCGCTCAGATCCTGATCAGCCTGGAGAGCGAG |
| | | GAGAGAGGCGAGCTGGAGAGAATCCTAGCAGATCTGGAGGAAGAGAACAGAAACCTGCAAGCCG |
| | | AGTATGACCGACTGAAACAGCAACATGAGCACAAAGGCCTGAGCCCACTGCCCTCCCCCCCCGAG |
| | | ATGATGCCCACAAGCCCTCAGAGCCCTAGAGACGCCGAGCTGATCGCTGAAGCGAAGTTACTTAGA |
| | | CAACACAAGGGCAGACTGGAGGCTAGAATGCAGATCCTGGAGGACCACAACAAGCAGCTGGAGT |
| | | CTCAGCTGCACAGACTGAGGCAGCTGCTGGAACAGCCCCAAGCCGAGGCCAAAGTGAACGGCAC |
| | | CACCGTGAGCAGTCCTAGCACAAGCCTGCAGAGAAGCGACAGCTCTCAGCCCATGCTGCTGAGAG |
| | | TGGTGGGCTCTCAGACAAGCGACAGCATGGGCGAGGAGGATCTGCTGAGTCCGCCCCAAGACACA |
| | | AGCACCGGCCTGGAAGAGGTGATGGAGCAACTGAACAATAGCTTCCCTAGCAGCAGAGGCAGAAA |
| | | CACCCCCGGCAAGCCCATGAGAGAGGACACCATGTGATGACTCGAGAATCAACCTCTGGATTACAA |
| | | AATTTGTGAAAGATTGACTGGTATTCTTAACTATGTTGCTCCTTTTACGCTATGTGGATACGCTGCTTT |
| | | AATGCCTTTGTATCATGCTATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGTGTGCACTGTGTTTGCTG |
| | | ACGCAACCCCCACTGGTTGGGGCATTGCCACCACCTGTCAGCTCCTTTCCGGGACTTTCGCTTTCCC |
| | | CCTCCCTATTGCCACGGCGGAACTCATCGCCGCCTGCCTTGCCCGCTGCTGGACAGGGGCTCGGCT |
| | | GTTGGGCACTGACAATTCCGTGGGGTGTGCCTTCTAGTTGCCAGCCATCTGTTGTTTGCCCCTCCCC |
| | | CGTGCCTTCCTTGACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGACAGCAAGGGGGAG |
| | | GATTGGGAAGACAATAGCAGGCATGCTGGGGATGCGGTGGGCTCTATGGGAAGATGTCTACTGAGC |
| | | TGTGCGATCCCTGCTGGGGACTTTCCGCTGGGGACTTTCCGCTGGGGACTTTCCGCCTTCAGCTAA |
| | | GGAAGCTACCAATATTTAGAGGTACATTTTGTTCTAGAACAAAATGTACCGGTACATTTTGTTCTGGT |
| | | ACATTTTGTTCTATTAAGGACAACGTAAGTATAGCGCATAGACACGTGGCGCCATCGGATCCCGGGC |
| | | CCGTGGCTTGTTGTCCACAACCATTAAACCTTAAAAGCTTTAAAAGCCTTATATATTCTTTTTTTTCTT |
| | | ATAAAACTTAAAACCTTAGAGGCTATTTAAGTTGCTGATTTATATTAATTTTATTGTTCAAACATGAGA |
| | | GCTTAGTACGTGAAACATGAGAGCTTAGTACATTAGCCATGAGAGCTTAGTACATTAGCCATGAGGG |
| | | TTTAGTTCATTAAACATGAGAGCTTAGTACATTAAACATGAGAGCTTAGTACATTAAACATGAGAGC |
| | | TTAGTACATACTATCAACAGGTTGAACTGCTGATCTGTACAGTAGAATTGGTAAAGAGAGTTGTGTA |
| | | AAATATTGAGTTCGCACATCTTGTTGTCTGATTATTGATTTTTGGCGAAACCATTTGATCATATGACAA |
| | | GATGTGTATCTACCTTAACTTAATGATTTTGATAAAAATCATTATGCGGCCGCCGTTACATAACTTACG |
| | | GTAAATGGCCCGCCTGGCTGACCGCCCAACGACCCCCGCCCATTGACGTCAATAATGACGTATGTTC |
| | | CCATAGTAACGCCAATAGGGACTTTCCATTGACGTCAATGGGTGGAGTATTTACGGTAAACTGCCCA |
| | | CTTGGCAGTACATCAAGTGTATCATATGCCAAGTACGCCCCCTATTGACGTCAATGACGGTAAATGG |
| | | CCCGCCTGGCATTATGCCCAGTACATGACCTTATGGGACTTTCCTACTTGGCAGTACATCTACGTATT |
| | | AGTCATCGCTATTACCATGGTCGAGGTGAGCCCCACGTTCTGCTTCACTCTCCCCATCTCCCCCCCCT |
| | | CCCCACCCCCAATTTTGTATTTATTTATTTTTTAATTATTTTGTGCAGCGATGGGGGGGGGGGGGGG |
| | | GGGGCGCGCGCCAGGCGGGGGGGGCGGGGCGAGGGGCGGGGCGGGGCGAGGCGGAGAGGTGC |
| | | GGCGGCAGCCAATCAGAGCGGCGCGCTCCGAAAGTTTCCTTTTATGGCGAGGCGGCGGCGGCGGC |
| | | GGCCCTATAAAAAGCGAAGCGCGCGGCGGGCGGGAGTC |
| 15. | NP-CBA- | TCTGGATCCACTGCTTAAATACGGACGAGGACAGGGCCCTGTCTCCTCAGCTTCAGGCACCACCAC |
| | hBGi-DMD- | TGACCTGGGACAGTGAATCGTAAGTACTAGCAGCTACAATCCAGCTACCATTCTGCTTTTATTTTATG |
| | 4-WPRE3- | GTTGGGATAAGGCTGGATTATTCTGAGTCCAAGCTAGGCCCTTTTGCTAATCATGTTCATACCTCTTA |
| | DTS | TCTTCCTCCCACAGCTCCTGGGCAACGTGCTGGTCTGTGTGCTGGCCCATCACTTTGGCAAAGAATT |
| | | GCGATCGCCACCATGCTTTGGTGGGAAGAAGTCGAGGACTGCTACGAGCGCGAGGACGTGCAGAA |
| | | GAAAACCTTCACCAAATGGGTCAACGCCCAGTTCAGCAAGTTCGGCAAGCAGCACATCGAGAACC |
| | | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACCTAGCTGGCTTGTTGTCCACAACCATTAAACCTTAAAAGCTTTAAAAGCCTTATA |
| | | TATTCTTTTTTTTCTTATAAAACTTAAAACCTTAGAGGCTATTTAAGTTGCTGATTTATATTAATTTTAT |
| | | TGTTCAAACATGAGAGCTTAGTACGTGAAACATGAGAGCTTAGTACATTAGCCATGAGAGCTTAGTA |
| | | CATTAGCCATGAGGGTTTAGTTCATTAAACATGAGAGCTTAGTACATTAAACATGAGAGCTTAGTAC |
| | | ATTAAACATGAGAGCTTAGTACATACTATCAACAGGTTGAACTGCTGATCTGTACAGTAGAATTGGT |
| | | AAAGAGAGTTGTGTAAAATATTGAGTTCGCACATCTTGTTGTCTGATTATTGATTTTTGGCGAAACC |
| | | ATTTGATCATATGACAAGATGTGTATCTACCTTAACTTAATGATTTTGATAAAAATCATTACGAATGCA |
| | | TGCGGCCGCCGTTACATAACTTACGGTAAATGGCCCGCCTGGCTGACCGCCCAACGACCCCCGCCC |
| | | ATTGACGTCAATAATGACGTATGTTCCCATAGTAACGCCAATAGGGACTTTCCATTGACGTCAATGGG |
| | | TGGAGTATTTACGGTAAACTGCCCACTTGGCAGTACATCAAGTGTATCATATGCCAAGTACGCCCCC |
| | | TATTGACGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTCGAGGTGAGCCCCACGTTCTGC |
| | | TTCACTCTCCCCATCTCCCCCCCCTCCCCACCCCCAATTTTGTATTTATTTATTTTTTAATTATTTTGTG |
| | | CAGCGATGGGGGCGGGGGGGGGGGGGGCGCGCGCCAGGCGGGGCGGGGCGGGGCGAGGGGCGG |
| | | GGCGGGGCGAGGCGGAGAGGTGCGGCGGCAGCCAATCAGAGCGGCGCGCTCCGAAAGTTTCCTTT |
| | | TATGGCGAGGCGGCGGCGGCGGCGGCCCTATAAAAAGCGAAGCGCGCGGCGGGCGGGAGTC |
| 16. | NP- | TCTGGATCCACTGCTTAAATACGGACGAGGACAGGGCCCTGTCTCCTCAGCTTCAGGCACCACCAC |
| | MHCK7- | TGACCTGGGACAGTGAATCGTAAGTACTAGCAGCTACAATCCAGCTACCATTCTGCTTTTATTTTATG |
| | hBGi-DMD- | GTTGGGATAAGGCTGGATTATTCTGAGTCCAAGCTAGGCCCTTTTGCTAATCATGTTCATACCTCTTA |
| | 3-WPRE3- | TCTTCCTCCCACAGCTCCTGGGCAACGTGCTGGTCTGTGTGCTGGCCCATCACTTTGGCAAAGAATT |
| | DTS | GCGATCGCCACCATGCTGTGGTGGGAAGAGGTGGAGGACTGTTACGAGAGAGAGGACGTGCAGAA |
| | | GAAGACATTCACCAAGTGGGTGAACGCTCAGTTCAGCAAGTTCGGCAAGCAGCACATCGAGAACC |
| | | TGTTTAGCGATCTACAAGATGGCAGAAGACTGCTGGACCTGCTGGAGGGCCTGACCGGGCAGAAG |
| | | CTGCCCAAAGAAAAGGGGTCTACAAGAGTGCACGCCCTGAACAACGTGAACAAGGCCCTGAGAG |
| | | TGCTGCAGAACAACAACGTGGACCTGGTGAACATCGGCAGCACCGACATCGTGGACGGCAACCAC |
| | | AAGCTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATCCTGCTGAGCTGGGTGAGACAGAGCA |
| | | CAAGAAACTACCCCCAAGTGAACGTGATCAACTTCACCACAAGCTGGAGCGACGGCCTGGCCCTG |
| | | AACGCCCTGATCCACAGCCACAGACCCGACCTGTTCGACTGGAACAGCGTGGTGTGTCAGCAGAG |
| | | CGCCACACAGAGACTGGAGCACGCCTTCAACATCGCTAGATATCAGCTGGGCATCGAGAAGTTACT |
| | | GGACCCCGAAGATGTGGACACCACCTACCCCGACAAGAAGAGCATCCTGATGTACATCACAAGCCT |
| | | GTTCCAAGTGCTGCCTCAGCAAGTGAGCATCGAGGCCATCCAAGAAGTCGAGATGCTGCCTAGACC |
| | | CCCCAAGGTGACCAAGGAGGAGCACTTTCAGCTGCACCATCAGATGCACTACTCTCAGCAGATCAC |
| | | AGTCAGCTTGGCACAAGGCTACGAGAGAACAAGCAGCCCCAAGCCTAGATTCAAGAGCTACGCCT |
| | | ACACCCAAGCCGCCTACGTGACCACAAGTGACCCCACAAGAAGCCCCTTCCCTTCTCAGCACCTG |
| | | GAGGCCCCCGAGGACAAGAGCTTCGGCAGCAGCCTGATGGAGAGCGAGGTGAACCTGGACAGAT |
| | | ATCAGACCGCGTTAGAAGAAGTGTTGAGCTGGCTGCTGAGCGCCGAGGACACCCTGCAAGCCCAA |
| | | GGCGAGATCAGCAACGATGTGGAGGTGGTGAAGGATCAGTTCCACACCCACGAGGGCTACATGAT |
| | | GGACCTGACCGCCCACCAAGGCAGAGTGGGCAACATCCTGCAGCTGGGCAGCAAGCTGATCGGCA |
| | | CCGGCAAGCTGAGCGAGGACGAGGAAACCGAGGTTCAAGAGCAAATGAACCTGCTAAACAGCAG |
| | | ATGGGAGTGCCTGAGAGTGGCTAGCATGGAAAAGCAGAGTAACCTGCACAGAGTGCTGATGGACC |
| | | TGCAGAATCAGAAATTGAAAGAACTTAATGATTGGCTGACCAAGACCGAGGAGAGAACAAGAAAG |
| | | ATGGAGGAGGAGCCCCTGGGCCCCGACCTGGAGGACCTGAAGAGACAAGTGCAGCAGCACAAGG |
| | | TGCTGCAAGAGGACCTGGAGCAAGAGCAAGTGAGAGTGAACTCTCTGACACACATGGTGGTGGTA |
| | | GTGGACGAGAGCAGCGGCGATCACGCTACCGCGGCGCTGGAAGAGCAGCTGAAAGTGCTGGGCG |
| | | ACAGATGGGCCAACATCTGCAGATGGACCGAGGACAGATGGGTGCTGCTGCAAGACATCCTGCTG |
| | | AAGTGGCAGAGACTGACCGAGGAGCAGTGCCTGTTCAGCGCCTGGCTGAGCGAGAAGGAAGACG |
| | | CTGTGAACAAGATCCACACCACCGGCTTCAAGGATCAGAACGAGATGCTGAGCTCCCTCCAAAAG |
| | | CTCGCAGTGCTGAAGGCCGACCTGGAAAAGAAAAAACAGTCGATGGGCAAGCTGTACAGCCTGAA |
| | | GCAAGACCTGCTGAGCACCCTGAAGAACAAGAGCGTGACACAGAAGACCGAGGCCTGGCTGGAC |
| | | AACTTCGCTAGATGCTGGGACAACCTGGTACAGAAGTTAGAGAAGTCTACCGCTCAGATCAGCCAA |
| | | GCCGTAACCACCACACAACCTAGCCTGACTCAGACCACCGTGATGGAAACCGTGACCACCGTGAC |
| | | AACCCGTGAGCAGATCCTGGTGAAGCACGCCCAAGAGGAGCTGCCCCCCCCCCCCCCTCAGAAGA |
| | | AGAGACAGATCACCGTGGACTCAGAGATTCGCAAGAGACTGGACGTGGACATCACCGAGCTGCAC |
| | | AGCTGGATCACAAGAAGCGAGGCCGTGCTGCAGAGCCCTGAGTTCGCGATCTTTAGAAAGGAGGG |
| | | CAACTTCAGCGACTTGAAGGAGAAAGTGAACGCCATCGAGAGAGAGAAGGCCGAGAAGTTCAGA |
| | | AAGTTGCAAGACGCCTCAAGAAGCGCCCAAGCCCTGGTGGAGCAGATGGTTAACGAAGGCGTGAA |
| | | TGCCGACAGCATCAAGCAAGCTAGCGAGCAGCTGAATAGCCGGTGGATTGAATTCTGTCAGCTGCT |
| | | GAGCGAGAGATTAAACTGGCTGGAGTATCAGAACAACATCATCGCCTTCTACAATCAGCTGCAGCA |
| | | ACTAGAACAAATGACCACCACCGCCGAGAACTGGCTAAAGATTCAGCCCACCACCCCTAGCGAGC |
| | | CCACCGCCATCAAATCTCAGCTGAAAATTTGCAAGGACGAAGTGAACAGACTGTCGGACCTGCAG |
| | | CCTCAAATCGAAAGACTGAAAATTCAGAGCATCGCACTTAAGGAGAAGGGCCAAGGACCCATGTT |
| | | CCTGGACGCCGACTTCGTGGCCTTCACCAACCACTTCAAGCAAGTGTTCAGCGACGTGCAAGCTAG |
| | | AGAGAAGGAGCTGCAGACCATCTTCGACACCCTGCCCCCCATGAGATACCAAGAAACCATGAGCG |
| | | CCATCAGAACCTGGGTGCAGCAGAGCGAAACCAAGCTGAGCATCCCTCAGCTGAGCGTGACCGAC |
| | | TACGAGATCATGGAGCAAAGACTGGGAGAGCTGCAAGCCCTGCAGAGCAGCCTGCAAGAGCAGC |
| | | AGAGCGGCCTGTACTACCTGAGCACCACCGTGAAGGAGATGTCGAAGAAAGCCCCGTCGGAGATC |
| | | AGCAGAAAGTATCAGAGCGAGTTCGAGGAGATCGAGGGCAGATGGAAGAAGCTGAGCAGCCAAC |
| | | TGGTGGAACACTGTCAGAAATTAGAAGAACAGATGAACAAGCTGAGAAAGATTCAGAACCACATT |
| | | CAGACCCTGAAGAAGTGGATGGCCGAGGTGGACGTGTTCCTGAAAGAGGAGTGGCCCGCCCTGGG |
| | | CGATTCCGAAATCTTAAAAAAGCAGCTGAAGCAGTGCAGACTGCTGGTGTCAGATATCCAAACCAT |
| | | TCAACCAAGCCTGAACAGCGTGAACGAAGGCGGGCAGAAGATCAAGAACGAGGCCGAGCCCGAG |
| | | TTCGCTAGCAGACTGGAAACCGAGCTGAAGGAGCTGAATACACAGTGGGACCACATGTGTCAGCA |
| | | AGTGTACGCTAGAAAGGAAGCGCTGAAGGGCGGGCTGGAGAAGACCGTTAGCCTGCAAAAGGATC |
| | | TGAGCGAGATGCACGAGTGGATGACCCAAGCCGAGGAGGAGTACCTGGAGAGAGACTTCGAGTAC |
| | | AAGACCCCCGACGAGCTGCAGAAGGCCGTCGAGGAAATGAAGAGAGCGAAGGAGGAGGCTCAGC |
| | | AGAAGGAAGCCAAGGTGAAGCTGCTGACTGAATCTGTCAACAGTGTGATAGCTCAAGCACCCCCC |
| | | GTTGCCCAAGAGGCACTAAAGAAGGAGCTGGAAACTCTGACCACCAACTATCAGTGGCTGTGCAC |
| | | AAGACTGAACGGCAAGTGCAAGACCCTGGAGGAAGTGTGGGCCTGCTGGCACGAGCTGCTGAGC |
| | | TACCTGGAGAAGGCCAACAAGTGGCTGAACGAGGTGGAGTTCAAGCTGAAGACCACCGAGAACA |
| | | TCCCCGGCGGCGCCGAGGAGATCAGCGAGGTGCTGGACAGCCTGGAGAACCTGATGAGACACAGC |
| | | GAGGACAACCCCAATCAGATCAGAATCCTGGCTCAGACCCTGACCGACGGCGGCGTGATGGACGA |
| | | GCTGATCAACGAGGAGCTGGAAACCTTTAACTCTAGATGGCGAGAACTGCACGAGGAGGCCGTGA |
| | | GAAGACAGAAGCTGCTGGAGCAAAGCATTCAGAGCGCCCAAGAAACCGAGAAGAGCCTGCACCT |
| | | GATCCAAGAGAGCCTGACCTTCATCGACAAGCAGCTGGCCGCCTACATCGCCGACAAGGTGGACG |
| | | CCGCTCAGATGCCACAAGAGGCTCAGAAGATTCAGTCCGATCTGACAAGCCACGAGATCAGCCTG |
| | | GAGGAGATGAAAAAACACAACCAAGGCAAGGAGGCCGCTCAGAGAGTGCTGTCTCAGATCGACG |
| | | TGGCTCAGAAGAAGCTGCAAGACGTGAGCATGAAGTTCAGACTGTTTCAGAAGCCCGCCAACTTC |
| | | GAGCAGCGACTGCAAGAGAGCAAGATGATCCTGGACGAGGTGAAGATGCACCTGCCCGCCCTGGA |
| | | AACCAAGAGCGTGGAGCAAGAGGTGGTGCAGTCTCAGTTGAACCACTGCGTGAACCTGTACAAGA |
| | | GCCTGTCGGAGGTCAAGAGTGAGGTCGAGATGGTGATCAAAACTGGGAGACAGATCGTGCAAAAA |
| | | AAGCAGACCGAGAACCCCAAGGAGCTGGACGAGCGTGTTACCGCCCTGAAGCTGCACTACAACGA |
| | | GCTGGGCGCCAAGGTGACCGAGAGAAAGCAGCAGCTGGAGAAGTGCCTGAAGCTGAGCAGAAAG |
| | | ATGAGAAAGGAGATGAACGTGCTGACCGAGTGGCTGGCCGCCACCGACATGGAGCTGACCAAGAG |
| | | AAGCGCCGTGGAGGGCATGCCTAGCAACCTGGACAGCGAGGTGGCCTGGGGCAAGGCCACACAG |
| | | AAGGAGATCGAGAAGCAGAAGGTGCACCTGAAGAGCATCACCGAGGTGGGCGAGGCCCTGAAGA |
| | | CCGTGCTGGGCAAGAAGGAAACCCTGGTGGAGGACAAGCTGAGCCTGCTGAACTCGAACTGGATC |
| | | GCCGTGACAAGCAGAGCCGAGGAGTGGCTGAACCTGCTGTTAGAGTACCAAAAACACATGGAAAC |
| | | CTTCGATCAGAACGTGGACCACATCACCAAGTGGATCATCCAAGCCGACACCCTGCTGGACGAAA |
| | | GCGAAAAGAAGAAGCCGCAACAAAAGGAGGATGTGCTGAAGAGACTGAAGGCCGAACTGAACG |
| | | ATATCAGACCCAAGGTGGACAGCACACGGGACCAAGCCGCCAACCTGATGGCCAACAGAGGCGAC |
| | | CACTGCAGAAAACTGGTGGAGCCTCAGATCAGCGAGCTGAACCACAGATTCGCCGCCATCAGCCA |
| | | CAGAATCAAAACCGGCAAAGCGAGCATCCCCTTGAAGGAACTGGAGCAATTTAACAGCGACATCC |
| | | AAAAATTGCTCGAACCACTGGAGGCCGAGATTCAGCAAGGCGTGAACCTGAAGGAAGAGGACTTC |
| | | AACAAGGACATGAACGAGGACAACGAGGGCACCGTGAAAGAACTGCTGCAGAGAGGCGACAACC |
| | | TGCAGCAGAGAATCACCGACGAGAGAAAGAGAGAGGAGATCAAGATCAAGCAGCAGCTGCTGCA |
| | | GACTAAGCACAACGCCCTGAAGGATCTGAGATCTCAGAGGAGAAAAAAGGCTCTGGAAATTTCAC |
| | | ATCAGTGGTATCAGTACAAGAGACAAGCAGACGACCTGCTGAAGTGCCTGGACGACATCGAGAAG |
| | | AAGTTAGCTAGCCTGCCCGAGCCTAGAGACGAGAGAAAGATCAAGGAGATCGACAGAGAGTTACA |
| | | AAAGAAGAAGGAGGAGCTGAATGCGGTAAGAAGACAAGCGGAAGGCCTGTCCGAGGACGGCGCA |
| | | GCCATGGCCGTGGAGCCCACACAGATTCAGCTCAGCAAGAGATGGAGAGAGATCGAGAGCAAGTT |
| | | CGCTCAGTTCAGAAGACTGAACTTCGCTCAGATCCACACCGTGAGAGAGGAAACCATGATGGTGAT |
| | | GACCGAGGACATGCCCCTGGAGATCTCATACGTTCCTAGCACCTACCTGACCGAGATCACCCACGT |
| | | GAGCCAAGCCCTGCTGGAGGTGGAGCAGCTGCTGAACGCCCCCGACCTGTGCGCCAAGGACTTCG |
| | | AGGACCTGTTCAAACAAGAGGAGAGCCTGAAGAACATCAAGGACAGCCTGCAGCAAAGTAGCGG |
| | | CAGAATCGACATCATCCACAGCAAGAAGACCGCCGCCCTGCAGAGCGCCACCCCCGTGGAGAGAG |
| | | TGAAGCTTCAAGAGGCCCTTTCGCAGCTGGATTTTCAGTGGGAGAAGGTGAACAAAATGTACAAG |
| | | GACAGACAAGGCAGATTCGACAGAAGCGTGGAGAAGTGGAGAAGATTCCACTACGACATCAAGAT |
| | | CTTCAATCAGTGGCTGACCGAGGCAGAGCAGTTCCTGAGAAAGACACAGATCCCCGAGAACTGGG |
| | | AGCACGCCAAGTACAAGTGGTACCTGAAGGAGCTGCAAGACGGCATCGGGCAGAGACAGACCGT |
| | | GGTGAGAACCCTGAACGCCACCGGCGAGGAGATCATCCAACAAAGCTCCAAAACCGACGCTAGCA |
| | | TCCTGCAAGAGAAACTGGGCAGCCTGAACCTGAGATGGCAAGAGGTGTGCAAGCAGCTGAGCGA |
| | | CCGCAAAAAACGGTTAGAGGAGCAGAAGAACATCCTGAGCGAGTTTCAGCGGGACCTGAACGAG |
| | | TTCGTGCTGTGGCTGGAGGAGGCCGATAACATCGCTAGCATCCCCCTGGAGCCCGGCAAGGAGCAG |
| | | CAGTTAAAAGAAAAACTTGAGCAAGTGAAGCTGCTGGTGGAGGAGCTGCCCCTGAGACAAGGCAT |
| | | CCTGAAGCAGCTGAACGAGACTGGCGGCCCCGTGCTGGTGAGCGCCCCCATCAGCCCCGAGGAGC |
| | | AAGACAAGCTGGAGAACAAGCTGAAGCAGACCAACCTGCAGTGGATCAAGGTGAGCAGAGCCCT |
| | | GCCCGAGAAGCAAGGCGAAATCGAGGCCCAAATAAAGGACCTGGGGCAGCTGGAGAAGAAACTC |
| | | GAAGACTTGGAAGAACAACTCAACCACTTGCTGCTGTGGCTGAGCCCCATCAGAAATCAGCTGGA |
| | | GATCTACAATCAGCCCAACCAAGAGGGGCCATTCGACGTGAAGGAAACCGAGATCGCCGTGCAAG |
| | | CCAAGCAGCCCGATGTGGAGGAGATCCTGAGCAAGGGGCAGCACCTGTACAAGGAGAAGCCCGCC |
| | | ACACAGCCCGTCAAGCGTAAACTAGAGGATCTTTCCTCTGAGTGGAAGGCCGTGAACCGTCTGCTA |
| | | CAAGAGCTGCGGGCTAAGCAACCCGACTTAGCGCCCGGCCTGACCACCATCGGTGCAAGCCCCAC |
| | | GCAGACCGTGACCCTGGTGACCCAACCCGTGGTGACCAAGGAAACCGCCATCAGCAAGCTGGAGA |
| | | TGCCTAGCAGCCTGATGCTGGAGGTGCCCGCCCTGGCCGACTTCAACAGAGCCTGGACCGAGCTG |
| | | ACCGACTGGCTGAGCCTGCTGGACCAAGTGATCAAGTCTCAGAGAGTGATGGTGGGCGATCTGGA |
| | | GGACATCAATGAGATGATCATCAAGCAGAAGGCCACCATGCAAGACCTGGAGCAGAGAAGACCTC |
| | | AGCTGGAGGAGCTGATCACCGCCGCTCAGAACCTGAAAAACAAGACGAGCAACCAAGAGGCTAG |
| | | AACCATCATCACCGACAGAATCGAGAGAATTCAGAATCAGTGGGACGAGGTGCAAGAGCACCTGC |
| | | AGAACAGAAGACAGCAACTAAACGAAATGCTCAAGGACAGCACACAGTGGCTGGAGGCCAAGGA |
| | | GGAGGCGGAGCAAGTGTTGGGCCAAGCTAGAGCCAAGCTGGAGAGCTGGAAGGAGGGACCCTAC |
| | | ACCGTGGACGCCATTCAGAAGAAGATCACCGAAACCAAGCAGCTGGCCAAGGACCTGCGACAGTG |
| | | GCAGACCAACGTGGACGTGGCCAACGATCTTGCCCTGAAGCTGCTGCGCGACTATAGCGCCGACG |
| | | ACACAAGAAAGGTGCACATGATCACCGAGAACATCAACGCTAGCTGGCGTAGCATCCACAAGAGA |
| | | GTGAGCGAGAGAGAGGCGGCTTTGGAGGAAACCCATAGACTGCTGCAGCAGTTTCCCCTGGACCT |
| | | GGAGAAGTTCCTGGCCTGGCTGACCGAAGCCGAAACAACCGCCAACGTGCTGCAAGACGCCACA |
| | | AGAAAGGAGAGACTGCTGGAGGACAGCAAGGGCGTGAAGGAGCTGATGAAGCAGTGGCAAGACC |
| | | TGCAAGGCGAGATCGAAGCGCACACCGACGTGTACCACAACCTGGACGAGAACTCTCAGAAGATC |
| | | CTGAGAAGCCTGGAGGGCAGCGACGACGCCGTGCTGTTACAGAGAAGGCTGGACAACATGAACTT |
| | | CAAGTGGAGCGAGCTGAGAAAGAAGAGCCTGAACATCAGAAGCCACCTGGAGGCTAGCAGCGAT |
| | | CAGTGGAAGAGACTGCACCTGAGCCTGCAAGAGCTGCTGGTCTGGCTTCAGTTGAAGGACGATGA |
| | | GCTGAGCAGACAAGCCCCCATCGGCGGCGACTTCCCCGCCGTGCAGAAGCAGAACGACGTGCATA |
| | | GAGCCTTCAAGAGAGAGCTGAAGACCAAGGAGCCCGTGATCATGAGCACCCTGGAAACGGTGAG |
| | | AATCTTCCTGACCGAGCAGCCCCTGGAGGGCCTGGAGAAGCTGTACCAAGAGCCTAGAGAGCTGC |
| | | CCCCCGAGGAGAGAGCTCAGAACGTGACAAGACTGCTGAGAAAGCAAGCCGAAGAGGTGAACAC |
| | | CGAGTGGGAGAAGCTGAACCTGCACAGCGCCGACTGGCAGAGAAAGATCGACGAAACCCTGGAG |
| | | AGACTGAGAGAATTGCAAGAGGCGACCGACGAGCTGGACCTGAAGCTGAGACAAGCCGAGGTCA |
| | | TCAAGGGCAGCTGGCAGCCCGTGGGCGATCTATTAATTGACTCTCTACAAGACCACCTGGAGAAAG |
| | | TGAAGGCCCTGAGAGGCGAGATCGCCCCCCTGAAGGAGAACGTGAGCCACGTGAATGATCTGGCA |
| | | CGTCAGCTGACCACCCTGGGCATACAACTGTCCCCCTATAACCTGAGCACACTGGAGGATTTGAATA |
| | | CTAGATGGAAGCTGCTGCAAGTGGCCGTGGAGGACAGAGTAAGACAATTACATGAGGCCCACAGA |
| | | GACTTCGGCCCCGCTTCTCAGCACTTCCTGAGCACAAGCGTGCAAGGCCCCTGGGAGAGAGCCAT |
| | | TTCCCCCAACAAGGTCCCCTACTACATCAACCACGAGACACAGACCACCTGCTGGGACCACCCCAA |
| | | GATGACCGAGCTGTATCAGAGCCTCGCCGATCTGAACAATGTGAGATTCAGCGCCTACAGAACCGC |
| | | CATGAAGCTGAGAAGACTGCAGAAGGCCCTGTGCCTGGACTTATTAAGCCTGAGCGCCGCCTGCG |
| | | ACGCCCTGGATCAGCACAACCTGAAGCAAAACGACCAACCCATGGACATCCTGCAGATCATCAACT |
| | | GCCTCACCACCATATACGACAGACTGGAACAAGAGCACAACAACCTGGTGAACGTGCCCCTGTGC |
| | | GTGGACATGTGCCTGAACTGGCTGCTGAACGTGTACGACACCGGCAGAACCGGCAGAATCAGAGT |
| | | GCTGAGCTTCAAGACCGGCATCATCAGCCTGTGCAAGGCCCACCTGGAGGACAAATACAGATACCT |
| | | TTTCAAACAAGTGGCTAGCAGCACCGGCTTCTGCGACCAAAGAAGACTGGGTCTGCTGCTGCACG |
| | | ACAGCATTCAGATCCCTAGACAGCTGGGCGAGGTGGCTAGCTTCGGCGGCAGCAACATCGAGCCTA |
| | | GCGTGAGAAGCTGCTTTCAGTTCGCCAACAACAAGCCCGAGATAGAGGCAGCCCTCTTCTTAGACT |
| | | GGATGAGACTGGAGCCTCAGAGCATGGTATGGCTGCCGGTCCTGCACAGAGTGGCCGCCGCCGAA |
| | | ACCGCCAAGCACCAAGCCAAGTGCAACATCTGCAAGGAGTGCCCCATCATCGGCTTCAGATACAGA |
| | | AGCCTGAAGCACTTCAACTACGACATCTGTCAGAGCTGCTTCTTCAGCGGCAGAGTGGCCAAGGG |
| | | CCACAAGATGCACTACCCCATGGTGGAGTACTGCACCCCCACCACAAGCGGCGAGGACGTGAGAG |
| | | ACTTCGCCAAGGTGTTGAAGAATAAGTTTAGAACCAAGAGATACTTCGCCAAGCACCCTAGAATGG |
| | | GCTACCTGCCCGTGCAGACCGTGCTGGAGGGCGACAACATGGAAACCCCCGTGACCCTGATCAAC |
| | | TTCTGGCCCGTGGACAGCGCCCCCGCTAGCAGCCCTCAGCTGAGCCACGACGACACCCACAGCAG |
| | | AATCGAGCACTACGCTAGCAGACTGGCCGAGATGGAGAACAGCAACGGCAGCTACCTGAACGACA |
| | | GCATCAGCCCGAATGAGAGCATCGACGACGAGCACCTGCTGATTCAGCACTACTGTCAGAGCCTGA |
| | | ACCAAGACAGCCCCCTGTCTCAGCCTAGAAGCCCCGCTCAGATCCTGATCAGCCTGGAGAGCGAG |
| | | GAGAGAGGCGAGCTGGAGAGAATCCTAGCAGATCTGGAGGAAGAGAACAGAAACCTGCAAGCCG |
| | | AGTATGACCGACTGAAACAGCAACATGAGCACAAAGGCCTGAGCCCACTGCCCTCCCCCCCCGAG |
| | | ATGATGCCCACAAGCCCTCAGAGCCCTAGAGACGCCGAGCTGATCGCTGAAGCGAAGTTACTTAGA |
| | | CAACACAAGGGCAGACTGGAGGCTAGAATGCAGATCCTGGAGGACCACAACAAGCAGCTGGAGT |
| | | CTCAGCTGCACAGACTGAGGCAGCTGCTGGAACAGCCCCAAGCCGAGGCCAAAGTGAACGGCAC |
| | | CACCGTGAGCAGTCCTAGCACAAGCCTGCAGAGAAGCGACAGCTCTCAGCCCATGCTGCTGAGAG |
| | | TGGTGGGCTCTCAGACAAGCGACAGCATGGGCGAGGAGGATCTGCTGAGTCCGCCCCAAGACACA |
| | | AGCACCGGCCTGGAAGAGGTGATGGAGCAACTGAACAATAGCTTCCCTAGCAGCAGAGGCAGAAA |
| | | CACCCCCGGCAAGCCCATGAGAGAGGACACCATGTGATGACTCGAGAATCAACCTCTGGATTACAA |
| | | AATTTGTGAAAGATTGACTGGTATTCTTAACTATGTTGCTCCTTTTACGCTATGTGGATACGCTGCTTT |
| | | AATGCCTTTGTATCATGCTATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGTGTGCACTGTGTTTGCTG |
| | | ACGCAACCCCCACTGGTTGGGGCATTGCCACCACCTGTCAGCTCCTTTCCGGGACTTTCGCTTTCCC |
| | | CCTCCCTATTGCCACGGCGGAACTCATCGCCGCCTGCCTTGCCCGCTGCTGGACAGGGGCTCGGCT |
| | | GTTGGGCACTGACAATTCCGTGGGGTGTGCCTTCTAGTTGCCAGCCATCTGTTGTTTGCCCCTCCCC |
| | | CGTGCCTTCCTTGACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGACAGCAAGGGGGAG |
| | | GATTGGGAAGACAATAGCAGGCATGCTGGGGATGCGGTGGGCTCTATGGGAAGATGTCTACTGAGC |
| | | TGTGCGATCCCTGCTGGGGACTTTCCGCTGGGGACTTTCCGCTGGGGACTTTCCGCCTTCAGCTAA |
| | | GGAAGCTACCAATATTTAGAGGTACATTTTGTTCTAGAACAAAATGTACCGGTACATTTTGTTCTGGT |
| | | ACATTTTGTTCTATTAAGGACAACGTAAGTATAGCGCATAGACACGTGGCGCCATCGGATCCCGGGC |
| | | CCGTGGCTTGTTGTCCACAACCATTAAACCTTAAAAGCTTTAAAAGCCTTATATATTCTTTTTTTTCTT |
| | | ATAAAACTTAAAACCTTAGAGGCTATTTAAGTTGCTGATTTATATTAATTTTATTGTTCAAACATGAGA |
| | | GCTTAGTACGTGAAACATGAGAGCTTAGTACATTAGCCATGAGAGCTTAGTACATTAGCCATGAGGG |
| | | TTTAGTTCATTAAACATGAGAGCTTAGTACATTAAACATGAGAGCTTAGTACATTAAACATGAGAGC |
| | | TTAGTACATACTATCAACAGGTTGAACTGCTGATCTGTACAGTAGAATTGGTAAAGAGAGTTGTGTA |
| | | AAATATTGAGTTCGCACATCTTGTTGTCTGATTATTGATTTTTGGCGAAACCATTTGATCATATGACAA |
| | | GATGTGTATCTACCTTAACTTAATGATTTTGATAAAAATCATTATCTAGAAGCTGCATGTCTAAGCTAG |
| | | ACCCTTCAGATTAAAAATAACTGAGGTAAGGGCCTGGGTAGGGGAGGTGGTGTGAGACGCTCCTGT |
| | | CTCTCCTCTATCTGCCCATCGGCCCTTTGGGGAGGAGGAATGTGCCCAAGGACTAAAAAAAGGCCA |
| | | TGGAGCCAGAGGGGCGAGGGCAACAGACCTTTCATGGGCAAACCTTGGGGCCCTGCTGTCTAGCA |
| | | TGCCCCACTACGGGTCTAGGCTGCCCATGTAAGGAGGCAAGGCCTGGGGACACCCGAGATGCCTG |
| | | GTTATAATTAACCCAGACATGTGGCTGCCCCCCCCCCCCCAACACCTGCTGCCTCTAAAAATAACCC |
| | | TGTCCCTGGTGGATCCCCTGCATGCGAAGATCTTCGAACAAGGCTGTGGGGGACTGAGGGCAGGCT |
| | | GTAACAGGCTTGGGGGCCAGGGCTTATACGTGCCTGGGACTCCCAAAGTATTACTGTTCCATGTTCC |
| | | CGGCGAAGGGCCAGCTGTCCCCCGCCAGCTAGACTCAGCACTTAGTTTAGGAACCAGTGAGCAAG |
| | | TCAGCCCTTGGGGCAGCCCATACAAGGCCATGGGGCTGGGCAAGCTGCACGCCTGGGTCCGGGGT |
| | | GGGCACGGTGCCCGGGCAACGAGCTGAAAGCTCATCTGCTCTCAGGGGCCCCTCCCTGGGGACAG |
| | | CCCCTCCTGGCTAGTCACACCCTGTAGGCTCCTCTATATAACCCAGGGGCACAGGGGCTGCCCTCAT |
| | | TCTACCACCACCTCCACAGCAC |
| 17. | NP- | TCTGGATCCACTGCTTAAATACGGACGAGGACAGGGCCCTGTCTCCTCAGCTTCAGGCACCACCAC |
| | MHCK7- | TGACCTGGGACAGTGAATCGTAAGTACTAGCAGCTACAATCCAGCTACCATTCTGCTTTTATTTTATG |
| | hBGi-DMD- | GTTGGGATAAGGCTGGATTATTCTGAGTCCAAGCTAGGCCCTTTTGCTAATCATGTTCATACCTCTTA |
| | 4-WPRE3- | TCTTCCTCCCACAGCTCCTGGGCAACGTGCTGGTCTGTGTGCTGGCCCATCACTTTGGCAAAGAATT |
| | DTS | GCGATCGCCACCATGCTTTGGTGGGAAGAAGTCGAGGACTGCTACGAGCGCGAGGACGTGCAGAA |
| | | GAAAACCTTCACCAAATGGGTCAACGCCCAGTTCAGCAAGTTCGGCAAGCAGCACATCGAGAACC |
| | | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACCTAGCTGGCTTGTTGTCCACAACCATTAAACCTTAAAAGCTTTAAAAGCCTTATA |
| | | TATTCTTTTTTTTCTTATAAAACTTAAAACCTTAGAGGCTATTTAAGTTGCTGATTTATATTAATTTTAT |
| | | TGTTCAAACATGAGAGCTTAGTACGTGAAACATGAGAGCTTAGTACATTAGCCATGAGAGCTTAGTA |
| | | CATTAGCCATGAGGGTTTAGTTCATTAAACATGAGAGCTTAGTACATTAAACATGAGAGCTTAGTAC |
| | | ATTAAACATGAGAGCTTAGTACATACTATCAACAGGTTGAACTGCTGATCTGTACAGTAGAATTGGT |
| | | AAAGAGAGTTGTGTAAAATATTGAGTTCGCACATCTTGTTGTCTGATTATTGATTTTTGGCGAAACC |
| | | ATTTGATCATATGACAAGATGTGTATCTACCTTAACTTAATGATTTTGATAAAAATCATTACGAATGCA |
| | | TCTAGAAGCTGCATGTCTAAGCTAGACCCTTCAGATTAAAAATAACTGAGGTAAGGGCCTGGGTAG |
| | | GGGAGGTGGTGTGAGACGCTCCTGTCTCTCCTCTATCTGCCCATCGGCCCTTTGGGGAGGAGGAAT |
| | | GTGCCCAAGGACTAAAAAAAGGCCATGGAGCCAGAGGGGCGAGGGCAACAGACCTTTCATGGGC |
| | | AAACCTTGGGGCCCTGCTGTCTAGCATGCCCCACTACGGGTCTAGGCTGCCCATGTAAGGAGGCAA |
| | | GGCCTGGGGACACCCGAGATGCCTGGTTATAATTAACCCAGACATGTGGCTGCCCCCCCCCCCCCA |
| | | ACACCTGCTGCCTCTAAAAATAACCCTGTCCCTGGTGGATCCCCTGCATGCGAAGATCTTCGAACAA |
| | | GGCTGTGGGGGACTGAGGGCAGGCTGTAACAGGCTTGGGGGCCAGGGCTTATACGTGCCTGGGAC |
| | | TCCCAAAGTATTACTGTTCCATGTTCCCGGCGAAGGGCCAGCTGTCCCCCGCCAGCTAGACTCAGC |
| | | ACTTAGTTTAGGAACCAGTGAGCAAGTCAGCCCTTGGGGCAGCCCATACAAGGCCATGGGGCTGG |
| | | GCAAGCTGCACGCCTGGGTCCGGGGTGGGCACGGTGCCCGGGCAACGAGCTGAAAGCTCATCTGC |
| | | TCTCAGGGGCCCCTCCCTGGGGACAGCCCCTCCTGGCTAGTCACACCCTGTAGGCTCCTCTATATAA |
| | | CCCAGGGGCACAGGGGCTGCCCTCATTCTACCACCACCTCCACAGCAC |
| 18. | CBA | CGTTACATAACTTACGGTAAATGGCCCGCCTGGCTGACCGCCCAACGACCCCCGCCCATTGACGTC |
| | | AATAATGACGTATGTTCCCATAGTAACGCCAATAGGGACTTTCCATTGACGTCAATGGGTGGAGTATT |
| | | TACGGTAAACTGCCCACTTGGCAGTACATCAAGTGTATCATATGCCAAGTACGCCCCCTATTGACGT |
| | | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGGGGGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | C |
| 19. | DMD-1 | ATGCTGTGGTGGGAAGAGGTGGAGGATTGCTACGAGCGGGAGGATGTGCAGAAAAAGACTTTCAC |
| | | CAAGTGGGTAAATGCTCAGTTCTCTAAGTTCGGGAAGCAACACATCGAGAACTTGTTTTCCGACCT |
| | | CCAGGATGGCAGGCGCCTGCTTGACTTGCTGGAGGGTCTGACTGGTCAGAAGCTCCCTAAGGAGA |
| | | AGGGCAGCACCAGAGTTCACGCTCTGAATAACGTTAACAAAGCCCTGAGGGTCCTCCAGAATAAC |
| | | AATGTAGACCTGGTGAACATCGGTTCTACCGACATCGTGGACGGGAATCATAAACTGACCTTGGGG |
| | | CTCATCTGGAACATTATCTTGCACTGGCAGGTGAAGAACGTCATGAAGAATATCATGGCCGGGCTGC |
| | | AACAGACCAACTCCGAAAAGATTTTGCTCAGCTGGGTGCGCCAGTCCACCCGCAATTATCCACAAG |
| | | TCAACGTTATCAATTTCACCACTAGTTGGAGTGATGGCCTCGCCCTGAACGCCTTGATCCACAGTCA |
| | | CCGCCCCGACCTGTTTGACTGGAACTCTGTGGTTTGCCAACAGAGCGCTACCCAGCGTCTCGAACA |
| | | TGCTTTCAATATCGCTAGATACCAGCTCGGTATCGAGAAGCTGTTGGATCCTGAGGACGTAGACACG |
| | | ACCTACCCCGATAAAAAGAGCATCCTGATGTATATCACTTCTCTGTTTCAGGTGCTTCCTCAACAGG |
| | | TGTCCATTGAAGCCATCCAAGAGGTCGAAATGCTGCCCAGGCCCCCTAAGGTGACAAAAGAAGAG |
| | | CATTTCCAGCTGCATCACCAGATGCATTACAGTCAACAGATTACCGTCAGCTTGGCTCAGGGATATG |
| | | AGCGCACGTCCTCTCCTAAGCCCAGATTTAAGTCCTACGCATATACCCAGGCTGCCTACGTGACCAC |
| | | TTCCGACCCAACTAGGAGCCCCTTTCCCTCCCAACACCTGGAGGCCCCTGAGGACAAAAGCTTCGG |
| | | ATCTAGTCTGATGGAGTCCGAGGTGAATCTGGATCGGTACCAAACTGCTCTGGAGGAAGTGCTGTC |
| | | CTGGCTTCTGTCCGCTGAAGACACCCTGCAGGCGCAAGGTGAGATTTCTAACGACGTCGAGGTCGT |
| | | GAAGGATCAGTTCCACACTCATGAGGGCTACATGATGGACTTGACAGCTCACCAGGGAAGAGTTGG |
| | | TAACATCCTGCAGCTTGGCTCCAAGTTGATTGGCACGGGAAAGCTTAGCGAGGACGAGGAAACCG |
| | | AGGTACAGGAGCAGATGAACTTGCTGAATTCCCGCTGGGAGTGCCTGAGAGTTGCGTCCATGGAA |
| | | AAGCAGTCAAACCTTCACAGAGTGCTGATGGACCTTCAGAACCAGAAGCTGAAGGAGCTGAACGA |
| | | TTGGCTGACAAAGACCGAGGAACGCACAAGAAAAATGGAAGAGGAACCTCTGGGACCTGATCTG |
| | | GAGGATCTGAAGCGCCAGGTCCAACAGCATAAGGTGCTGCAGGAGGATTTGGAGCAGGAGCAGGT |
| | | GCGGGTCAACAGCCTGACACATATGGTGGTTGTGGTCGATGAGTCCTCTGGCGATCATGCTACCGC |
| | | AGCGCTGGAAGAGCAGCTGAAGGTGCTGGGGGATCGCTGGGCCAACATTTGCAGATGGACAGAAG |
| | | ACCGGTGGGTCCTGCTTCAAGATATCCTGCTCAAGTGGCAGCGCCTGACTGAAGAGCAGTGCCTGT |
| | | TCTCAGCTTGGTTGAGCGAAAAAGAGGACGCAGTTAACAAGATCCATACCACAGGCTTCAAGGATC |
| | | AGAATGAGATGCTGAGCTCACTGCAGAAGCTGGCTGTGCTGAAGGCCGACTTGGAGAAGAAAAAG |
| | | CAGTCCATGGGCAAACTGTATAGCCTGAAGCAGGACCTGCTCAGCACCCTGAAGAACAAAAGCGT |
| | | TACGCAGAAAACTGAGGCCTGGTTGGACAACTTTGCTCGCTGCTGGGATAACCTTGTGCAGAAGCT |
| | | GGAAAAGTCAACCGCACAGATCTCCCAGGCTGTGACCACTACGCAGCCTAGCTTGACCCAGACAA |
| | | CCGTTATGGAAACCGTGACAACCGTGACAACGAGAGAGCAAATCCTGGTTAAGCATGCCCAGGAG |
| | | GAACTGCCGCCACCCCCGCCTCAGAAGAAACGGCAGATTACGGTGGACAGCGAGATCCGCAAGAG |
| | | GCTGGACGTCGACATCACTGAGCTCCACTCATGGATCACGCGGAGTGAGGCCGTGCTGCAGTCTCC |
| | | CGAGTTCGCTATTTTCCGCAAGGAGGGAAACTTCTCTGACTTGAAAGAGAAGGTGAATGCCATCGA |
| | | GCGTGAAAAGGCAGAAAAGTTCAGAAAGCTGCAGGACGCCTCTCGCTCTGCGCAGGCCCTGGTGG |
| | | AGCAGATGGTCAACGAGGGTGTTAACGCTGACAGCATCAAACAGGCAAGCGAACAGCTGAACTCC |
| | | AGATGGATCGAATTTTGCCAACTGCTCTCCGAACGCCTCAACTGGTTGGAGTACCAGAACAATATTA |
| | | TCGCTTTTTACAACCAGCTGCAGCAACTGGAGCAGATGACAACTACCGCCGAAAACTGGCTGAAA |
| | | ATTCAGCCAACCACACCGTCCGAGCCAACTGCGATCAAGTCTCAGCTGAAGATCTGCAAGGACGA |
| | | AGTGAACCGTCTTAGCGACTTGCAACCCCAGATTGAACGGCTCAAGATTCAGTCTATTGCTCTGAA |
| | | GGAGAAGGGTCAGGGTCCGATGTTCCTGGATGCAGATTTTGTCGCATTTACCAATCACTTCAAACA |
| | | GGTGTTTAGTGACGTCCAGGCGCGCGAGAAAGAGCTGCAGACTATTTTTGACACCCTGCCTCCCAT |
| | | GAGGTACCAGGAGACAATGAGTGCCATTCGCACCTGGGTGCAACAGTCCGAGACGAAATTGTCAA |
| | | TCCCACAGCTGTCCGTGACAGACTACGAGATTATGGAGCAACGGTTGGGGGAGCTGCAGGCTCTTC |
| | | AGAGTTCCCTGCAGGAGCAACAGAGCGGACTGTACTATCTTTCCACTACCGTGAAGGAAATGAGTA |
| | | AGAAAGCCCCTTCCGAGATCAGCCGCAAATACCAGTCTGAATTCGAAGAGATCGAGGGACGCTGG |
| | | AAAAAGCTGTCAAGCCAGCTGGTCGAGCACTGTCAGAAGCTGGAAGAGCAGATGAACAAGCTGC |
| | | GTAAAATCCAGAACCATATTCAGACCCTCAAGAAATGGATGGCAGAAGTTGACGTGTTTCTGAAGG |
| | | AAGAGTGGCCTGCCCTCGGCGATAGCGAAATCCTTAAGAAACAACTCAAGCAGTGCCGCCTCCTG |
| | | GTGTCCGATATTCAGACAATCCAGCCTTCCCTGAACAGCGTTAATGAGGGCGGACAGAAAATTAAA |
| | | AACGAGGCCGAACCCGAGTTCGCTTCTCGCCTGGAGACTGAACTGAAGGAGCTGAATACCCAGTG |
| | | GGACCATATGTGTCAACAGGTCTACGCACGCAAAGAGGCACTGAAGGGCGGTCTGGAGAAGACTG |
| | | TCTCACTCCAGAAGGACCTGAGCGAGATGCACGAGTGGATGACCCAGGCCGAAGAGGAATACCTG |
| | | GAGCGTGATTTTGAGTATAAGACCCCAGACGAACTCCAGAAAGCCGTTGAAGAGATGAAGAGGGC |
| | | TAAGGAGGAAGCCCAACAGAAGGAGGCGAAAGTGAAGCTCCTGACAGAGTCCGTGAACTCCGTC |
| | | ATCGCTCAAGCTCCTCCCGTGGCTCAGGAGGCCCTCAAGAAAGAGCTGGAAACTCTGACCACAAA |
| | | TTACCAGTGGTTGTGTACCCGCTTGAACGGTAAGTGCAAGACCCTGGAAGAGGTGTGGGCCTGCTG |
| | | GCACGAGCTCCTGTCCTATCTTGAGAAGGCGAACAAATGGCTGAACGAAGTGGAATTCAAGTTGA |
| | | AAACAACTGAGAACATTCCGGGAGGCGCTGAGGAAATCTCTGAGGTGCTGGACTCTCTGGAAAAT |
| | | CTGATGCGTCATTCTGAAGACAATCCTAACCAGATCAGGATTCTGGCACAGACCCTCACTGACGGA |
| | | GGCGTTATGGACGAACTTATCAACGAGGAACTGGAGACATTCAATTCCCGTTGGCGTGAGCTGCAC |
| | | GAAGAGGCCGTGCGCCGTCAGAAGTTGCTGGAACAGTCTATCCAGTCCGCCCAGGAGACAGAGAA |
| | | ATCTCTCCACCTGATCCAGGAGAGTCTGACGTTTATCGATAAGCAGCTGGCTGCCTACATCGCCGAC |
| | | AAGGTGGACGCTGCCCAGATGCCCCAAGAGGCTCAGAAGATCCAGTCCGACCTGACGTCTCATGA |
| | | AATCAGCCTGGAAGAGATGAAAAAGCACAACCAGGGCAAAGAGGCTGCCCAGCGCGTCCTCTCCC |
| | | AGATTGACGTGGCTCAGAAAAAGTTGCAGGACGTGAGCATGAAGTTTAGGCTGTTCCAGAAACCC |
| | | GCCAACTTCGAACAGAGACTGCAGGAGTCTAAGATGATCCTGGATGAGGTCAAGATGCACCTCCCT |
| | | GCTCTGGAAACAAAATCTGTGGAGCAGGAGGTCGTGCAGTCACAACTGAACCATTGTGTGAACCT |
| | | CTACAAGTCTCTCAGCGAGGTGAAGTCCGAGGTCGAGATGGTGATCAAGACCGGACGCCAGATTG |
| | | TGCAGAAAAAGCAGACAGAGAACCCAAAGGAGCTGGACGAACGGGTGACCGCCCTGAAGCTCCA |
| | | CTACAACGAGTTGGGTGCCAAAGTCACTGAAAGAAAGCAACAGCTGGAGAAGTGTCTGAAGCTG |
| | | AGCCGCAAGATGCGCAAAGAAATGAACGTCTTGACCGAGTGGTTGGCGGCCACCGACATGGAGCT |
| | | GACCAAGAGATCCGCTGTGGAGGGCATGCCCTCCAACCTTGACTCAGAGGTGGCCTGGGGCAAGG |
| | | CCACACAGAAGGAGATCGAAAAACAGAAGGTGCATCTTAAATCAATCACTGAGGTGGGCGAAGCT |
| | | CTGAAGACAGTGCTGGGCAAGAAAGAGACACTTGTAGAGGACAAGCTCTCCCTTCTGAATAGCAA |
| | | TTGGATCGCCGTAACTAGTCGCGCGGAGGAATGGCTGAACCTCCTGTTGGAATACCAGAAACACAT |
| | | GGAAACCTTCGATCAGAACGTGGACCATATTACCAAGTGGATTATCCAGGCTGACACTCTCCTGGAC |
| | | GAGTCTGAGAAAAAGAAACCCCAGCAAAAGGAAGATGTGCTGAAGCGCCTGAAGGCGGAATTGA |
| | | ACGACATCCGCCCGAAGGTTGATTCCACCCGCGACCAAGCGGCCAACCTTATGGCTAACCGCGGGG |
| | | ACCATTGCAGAAAACTGGTGGAACCGCAGATCTCTGAGCTGAACCACAGATTCGCCGCGATTTCTC |
| | | ACAGAATTAAGACTGGCAAGGCCTCCATCCCTCTGAAAGAGCTGGAGCAGTTCAACTCTGATATTC |
| | | AGAAGCTTCTGGAGCCCCTGGAGGCAGAAATCCAACAGGGCGTCAACCTGAAAGAGGAAGACTTT |
| | | AATAAGGACATGAATGAGGATAACGAAGGTACCGTCAAGGAGCTCCTGCAGAGGGGCGACAATCT |
| | | GCAGCAAAGAATCACTGACGAACGTAAGCGCGAAGAGATTAAGATCAAGCAACAGCTGTTGCAGA |
| | | CTAAGCACAACGCCCTGAAGGATCTGAGGTCCCAGCGCAGGAAAAAGGCCCTGGAGATTTCCCAC |
| | | CAGTGGTACCAGTACAAGCGCCAGGCCGATGACCTTCTGAAGTGCCTCGACGATATCGAGAAAAAG |
| | | CTGGCCTCACTGCCCGAGCCCCGCGACGAGCGCAAGATCAAGGAGATTGACCGTGAGCTCCAGAA |
| | | GAAAAAGGAGGAACTGAACGCGGTGCGCAGACAGGCCGAGGGCCTCTCCGAGGACGGCGCTGCG |
| | | ATGGCTGTTGAGCCCACTCAGATCCAACTGAGCAAGCGGTGGAGAGAGATCGAGTCTAAGTTCGC |
| | | GCAATTTAGGCGCCTCAACTTCGCCCAGATTCACACGGTCCGTGAGGAAACGATGATGGTGATGAC |
| | | AGAGGATATGCCACTGGAAATCAGCTACGTGCCCAGTACATATCTCACCGAAATCACCCATGTGTCC |
| | | CAGGCTCTCCTGGAGGTGGAACAGTTGCTCAACGCTCCCGACTTGTGCGCTAAGGACTTCGAGGA |
| | | CCTGTTCAAGCAGGAAGAGAGCCTGAAAAACATTAAGGACTCCCTGCAACAGTCAAGCGGCCGCA |
| | | TCGATATCATTCATTCTAAGAAAACCGCTGCCCTGCAGTCTGCAACCCCCGTAGAGCGCGTCAAACT |
| | | GCAGGAAGCTCTCAGCCAACTGGATTTCCAGTGGGAGAAGGTGAACAAGATGTATAAGGATCGTC |
| | | AGGGCCGGTTCGACCGCTCTGTGGAGAAGTGGCGCCGTTTCCACTACGATATCAAGATTTTCAATC |
| | | AATGGTTGACCGAGGCAGAGCAGTTCCTCAGAAAGACACAGATCCCTGAAAATTGGGAGCATGCC |
| | | AAGTACAAGTGGTATCTGAAGGAGCTGCAGGACGGGATCGGACAACGTCAGACCGTGGTCCGCAC |
| | | CCTGAACGCAACCGGCGAGGAAATTATCCAACAGTCTAGCAAGACAGATGCTAGCATCCTCCAGGA |
| | | GAAGCTCGGAAGCCTCAACCTTCGCTGGCAGGAGGTGTGCAAGCAGCTGTCAGACAGGAAGAAA |
| | | CGCCTGGAGGAACAGAAGAACATCCTTTCAGAGTTCCAGAGAGATCTGAACGAATTCGTGCTTTGG |
| | | CTTGAAGAGGCCGATAACATCGCAAGCATCCCACTGGAGCCTGGTAAGGAACAGCAATTGAAGGA |
| | | GAAGCTGGAGCAGGTAAAGCTGCTCGTGGAAGAGCTGCCTCTGAGGCAGGGAATTCTCAAGCAGC |
| | | TCAACGAGACTGGGGGCCCGGTTTTGGTGAGCGCCCCAATTAGCCCAGAAGAGCAGGACAAGTTG |
| | | GAGAACAAGCTGAAGCAGACCAACCTGCAGTGGATCAAGGTGTCCCGCGCACTGCCCGAAAAGC |
| | | AGGGAGAGATTGAGGCCCAGATCAAGGATCTGGGACAGCTGGAGAAAAAGCTGGAAGACCTGGA |
| | | GGAACAGCTGAACCATTTGCTCCTGTGGTTGTCCCCCATCAGAAATCAGCTGGAAATCTACAACCA |
| | | ACCGAACCAGGAAGGTCCGTTTGATGTTAAGGAAACCGAGATCGCTGTGCAGGCGAAGCAGCCTG |
| | | ACGTAGAAGAGATCCTGTCAAAGGGACAGCACCTCTACAAGGAGAAGCCCGCCACCCAGCCAGTC |
| | | AAGCGGAAGCTCGAAGACCTGTCCAGCGAGTGGAAAGCCGTCAACCGCCTCCTGCAAGAGCTGC |
| | | GTGCCAAGCAACCGGACCTGGCGCCAGGTCTGACTACCATTGGCGCAAGTCCTACCCAGACTGTCA |
| | | CCCTGGTGACCCAACCCGTTGTGACAAAGGAAACCGCCATCAGCAAGCTGGAGATGCCTAGCTCTT |
| | | TGATGCTGGAGGTTCCCGCCCTCGCCGACTTCAACCGTGCTTGGACAGAACTGACCGACTGGCTGT |
| | | CCCTCCTGGACCAAGTGATCAAGAGCCAGCGCGTGATGGTGGGTGATCTTGAGGATATTAACGAAA |
| | | TGATTATCAAGCAGAAGGCCACCATGCAGGACTTGGAGCAGAGGCGCCCCCAGTTGGAGGAACTG |
| | | ATCACCGCCGCGCAGAATCTGAAAAACAAAACTTCCAATCAGGAAGCGAGAACCATCATTACAGA |
| | | CAGAATTGAGAGAATTCAAAACCAGTGGGACGAAGTGCAAGAACACCTCCAGAACCGGCGTCAA |
| | | CAGCTGAACGAGATGCTGAAGGATTCCACTCAATGGCTGGAAGCTAAGGAAGAGGCCGAGCAGGT |
| | | CCTTGGACAGGCACGCGCTAAGCTGGAGTCCTGGAAAGAAGGCCCCTATACAGTGGACGCAATTC |
| | | AAAAGAAAATCACCGAGACGAAGCAACTGGCCAAGGACCTGAGACAGTGGCAAACCAACGTTGA |
| | | TGTGGCTAATGACCTGGCCTTGAAGCTCCTGCGTGACTACTCCGCGGATGACACTAGAAAGGTGCA |
| | | CATGATCACCGAGAATATCAACGCTAGCTGGCGCTCCATCCATAAGCGTGTGAGTGAGCGCGAAGC |
| | | CGCTCTGGAGGAAACCCATCGCCTGCTTCAACAGTTTCCTTTGGACCTGGAAAAGTTCCTGGCCTG |
| | | GCTCACAGAAGCCGAGACAACCGCCAACGTGCTGCAGGACGCCACCCGTAAGGAGCGCTTGCTGG |
| | | AGGATAGTAAGGGGGTGAAGGAGTTGATGAAGCAGTGGCAAGATCTGCAGGGGGAAATCGAGGCA |
| | | CATACCGACGTCTATCATAACCTGGATGAGAATTCCCAGAAAATCCTGCGCAGCTTGGAGGGCAGT |
| | | GATGACGCGGTGCTTCTGCAACGGCGCTTGGACAACATGAACTTCAAGTGGTCCGAGTTGCGTAAA |
| | | AAGAGTCTGAACATTCGCTCCCACCTGGAGGCTTCTAGTGACCAGTGGAAGCGCCTGCACCTGAGC |
| | | CTGCAAGAACTGCTCGTGTGGCTGCAACTGAAGGATGACGAGTTGAGTCGTCAGGCCCCTATCGGA |
| | | GGTGACTTTCCGGCTGTCCAGAAACAGAACGACGTGCATAGGGCATTTAAGCGCGAGTTGAAGAC |
| | | AAAGGAGCCAGTCATTATGTCCACGCTGGAAACCGTGCGCATTTTCCTGACTGAGCAGCCTCTGGA |
| | | GGGGCTGGAGAAGCTGTACCAGGAGCCTCGCGAGTTGCCCCCTGAAGAGCGTGCCCAGAATGTGA |
| | | CCCGCCTGTTGCGTAAGCAGGCCGAGGAAGTGAACACCGAGTGGGAGAAGCTGAACCTGCATTCC |
| | | GCGGATTGGCAGAGGAAGATTGACGAGACGCTGGAGAGGCTCCGTGAGTTGCAGGAGGCAACCG |
| | | ACGAACTGGACCTCAAGCTGCGTCAAGCAGAGGTGATCAAGGGGAGTTGGCAGCCGGTCGGCGAT |
| | | TTGCTGATCGACTCCCTGCAGGATCACCTGGAAAAGGTTAAAGCTTTGCGCGGGGAAATTGCCCCG |
| | | CTGAAGGAGAACGTGAGCCACGTTAACGATCTGGCCAGACAGCTGACCACTCTGGGTATCCAGCT |
| | | GAGCCCGTACAATCTGAGCACCCTCGAAGACTTGAATACGAGATGGAAACTCCTGCAGGTGGCAGT |
| | | GGAGGACAGGGTGCGCCAATTGCACGAGGCCCACCGGGACTTTGGACCGGCCAGCCAGCATTTCC |
| | | TCAGTACCTCTGTTCAGGGCCCGTGGGAGCGGGCCATCTCCCCCAACAAGGTCCCCTATTACATCAA |
| | | CCATGAAACCCAGACTACCTGTTGGGACCACCCTAAAATGACTGAGTTGTATCAATCTCTGGCCGAC |
| | | TTGAATAACGTGCGCTTCTCAGCCTACAGGACTGCAATGAAACTCAGACGCCTCCAAAAGGCCCTT |
| | | TGTCTGGACTTGCTGTCCCTTAGCGCTGCCTGTGATGCTCTGGATCAGCACAATTTGAAACAAAACG |
| | | ACCAGCCGATGGACATCCTCCAGATTATCAACTGCCTGACTACCATCTACGACAGACTGGAACAGG |
| | | AGCATAATAACCTGGTGAACGTCCCCCTGTGCGTGGACATGTGCCTGAACTGGCTGCTCAATGTGTA |
| | | TGACACGGGTAGAACAGGACGGATCAGGGTCCTGTCCTTTAAGACCGGTATCATTTCTCTGTGTAA |
| | | GGCACATCTGGAGGACAAGTACCGCTACCTGTTTAAGCAGGTCGCCAGCTCCACTGGCTTCTGCGA |
| | | TCAAAGGCGGCTGGGTCTGCTCCTGCACGACTCCATTCAGATCCCGAGACAGTTGGGTGAAGTGGC |
| | | GTCCTTCGGCGGTTCTAATATCGAGCCTAGCGTCCGCAGCTGTTTCCAATTCGCCAATAACAAGCCG |
| | | GAGATCGAAGCGGCCCTTTTCCTGGACTGGATGCGTCTGGAACCTCAGAGCATGGTTTGGCTTCCG |
| | | GTGCTTCATCGCGTGGCCGCTGCCGAAACCGCTAAACATCAGGCCAAATGTAACATCTGTAAGGAA |
| | | TGCCCGATTATCGGTTTCCGGTACCGTAGCCTCAAGCATTTCAACTATGATATTTGTCAATCTTGCTTC |
| | | TTTAGCGGACGGGTGGCCAAAGGCCACAAAATGCACTACCCGATGGTCGAGTATTGCACTCCTACA |
| | | ACCTCCGGCGAGGACGTGCGCGATTTTGCCAAAGTGCTGAAAAACAAGTTTAGAACCAAGCGTTA |
| | | CTTTGCCAAGCATCCACGTATGGGCTACCTGCCTGTCCAGACCGTTCTGGAAGGTGATAACATGGAG |
| | | ACACCCGTGACTTTGATTAACTTTTGGCCAGTAGACTCTGCTCCGGCAAGCTCCCCTCAGCTCTCCC |
| | | ACGATGACACTCACAGCCGTATCGAGCACTACGCCTCTCGCCTGGCTGAGATGGAGAACTCTAACG |
| | | GTTCATACCTGAATGACAGTATCTCCCCAAACGAGAGTATCGATGACGAACATCTGCTCATTCAGCA |
| | | TTACTGTCAATCCTTGAATCAGGACAGCCCCCTGTCCCAGCCACGGTCCCCGGCCCAGATCCTGATT |
| | | TCACTGGAGTCCGAAGAGAGAGGGGAACTGGAAAGGATCCTCGCTGACCTGGAAGAGGAAAACC |
| | | GGAACCTGCAGGCTGAATACGACCGCTTGAAGCAGCAACACGAGCACAAGGGCTTGAGCCCGTTG |
| | | CCAAGCCCTCCGGAGATGATGCCAACTAGCCCTCAGAGCCCACGGGATGCCGAGCTCATCGCTGAA |
| | | GCTAAGTTGCTGAGACAGCACAAGGGACGTCTGGAGGCCCGCATGCAGATCCTGGAGGATCATAAT |
| | | AAACAGTTGGAGAGCCAACTGCATAGGCTGCGCCAGCTCCTGGAGCAGCCACAGGCCGAGGCTAA |
| | | GGTCAACGGAACTACCGTTTCCAGCCCGTCCACATCTCTGCAGCGCTCAGATAGTTCCCAGCCTATG |
| | | TTGCTGAGAGTGGTTGGATCTCAGACCTCCGACTCAATGGGCGAAGAGGACTTGCTGTCTCCCCCA |
| | | CAGGACACTAGCACAGGACTCGAAGAGGTGATGGAACAGCTCAATAACTCTTTTCCTTCTAGTCGG |
| | | GGTCGCAATACCCCTGGAAAGCCCATGCGCGAAGACACCATGTAATAG |
| 20. | DMD-5 | ATGCTGTGGTGGGAGGAAGTTGAAGATTGTTATGAGAGAGAAGATGTCCAGAAAAAGACCTTTACC |
| | | AAATGGGTGAACGCACAGTTCTCCAAATTTGGGAAACAGCATATAGAAAATTTGTTCAGCGATCTTC |
| | | AGGATGGGCGCAGGTTGCTTGACCTTCTGGAAGGACTGACCGGGCAGAAGCTGCCCAAAGAGAA |
| | | AGGGTCTACCAGAGTTCACGCTCTGAACAACGTGAATAAGGCACTGCGAGTCCTGCAAAATAACA |
| | | ACGTCGACCTGGTGAACATAGGCAGCACTGATATTGTTGATGGGAACCATAAACTCACACTGGGTC |
| | | TGATTTGGAATATTATACTGCACTGGCAGGTCAAAAATGTTATGAAAAATATTATGGCCGGGCTTCAA |
| | | CAGACCAATTCCGAGAAGATCCTGCTGTCTTGGGTTAGGCAGTCCACGAGGAACTACCCTCAAGTG |
| | | AACGTTATTAACTTCACAACATCATGGAGTGATGGACTTGCCCTGAACGCCTTGATCCACTCCCATA |
| | | GGCCAGACCTGTTCGATTGGAATAGCGTAGTCTGTCAGCAGAGTGCTACACAACGCCTGGAACACG |
| | | CCTTTAACATAGCCAGGTATCAGCTTGGAATTGAGAAACTCCTCGACCCTGAAGACGTAGACACCA |
| | | CTTACCCCGACAAAAAATCCATTCTTATGTACATAACTTCCTTGTTTCAGGTTCTCCCCCAGCAGGTG |
| | | TCCATCGAGGCTATACAGGAGGTCGAAATGCTCCCCAGACCCCCTAAGGTCACGAAGGAAGAGCA |
| | | CTTCCAACTGCACCATCAGATGCACTACAGTCAACAGATCACTGTGAGTCTCGCACAGGGGTACGA |
| | | AAGGACCAGCTCCCCTAAACCCCGCTTCAAGTCCTACGCCTATACTCAGGCAGCTTACGTAACCAC |
| | | AAGTGACCCCACGCGGTCACCCTTCCCAAGCCAGCACCTGGAGGCTCCCGAGGACAAAAGCTTTG |
| | | GATCATCCCTGATGGAATCTGAAGTCAATCTCGATAGGTACCAAACCGCTCTGGAGGAGGTCCTCA |
| | | GTTGGCTCCTTTCAGCTGAAGATACTCTGCAGGCACAGGGTGAGATCAGCAATGACGTGGAGGTAG |
| | | TGAAAGATCAGTTTCACACTCACGAGGGCTATATGATGGACCTGACAGCCCATCAAGGTCGCGTTG |
| | | GCAATATCCTGCAGTTGGGGTCCAAACTGATTGGTACCGGTAAGTTGTCCGAGGACGAAGAGACAG |
| | | AAGTGCAGGAGCAGATGAACCTGCTTAATTCTAGATGGGAATGCCTCCGCGTGGCCTCTATGGAGA |
| | | AGCAGTCCAATCTCCATCGGGTTCTCATGGACTTGCAGAATCAGAAGCTGAAGGAACTCAACGACT |
| | | GGTTGACCAAGACTGAGGAGCGCACCAGAAAGATGGAGGAGGAGCCACTCGGTCCTGATCTTGAA |
| | | GATCTGAAACGACAAGTTCAGCAGCACAAGGTCCTGCAGGAGGATCTCGAACAGGAGCAGGTCCG |
| | | GGTGAACAGCCTGACCCATATGGTTGTCGTTGTCGACGAGTCAAGTGGGGACCATGCTACGGCTGC |
| | | GCTTGAGGAACAGCTGAAGGTCTTGGGCGATAGATGGGCGAATATCTGCCGCTGGACTGAAGATAG |
| | | ATGGGTGCTGCTGCAGGATATTCTTTTGAAGTGGCAGCGGTTGACCGAAGAACAATGCTTGTTTAG |
| | | CGCCTGGCTGAGTGAGAAAGAGGATGCGGTGAATAAGATACACACTACTGGCTTCAAAGACCAGA |
| | | ATGAAATGCTTAGCAGCCTGCAGAAACTTGCGGTTCTTAAGGCCGACTTGGAAAAGAAAAAGCAG |
| | | TCAATGGGTAAGCTGTACTCACTCAAGCAGGACCTGCTGTCAACTTTGAAGAACAAGAGCGTCAC |
| | | GCAAAAGACTGAGGCCTGGCTCGACAATTTTGCTAGGTGCTGGGATAACCTGGTGCAGAAGTTGG |
| | | AAAAGTCCACTGCTCAGATCTCTCAAGCCGTGACTACTACCCAGCCCAGCCTGACACAGACTACCG |
| | | TCATGGAAACCGTTACCACTGTGACGACTAGGGAGCAGATACTCGTTAAGCACGCTCAAGAGGAAT |
| | | TGCCGCCACCTCCTCCCCAAAAGAAACGCCAGATTACCGTCGACTCTGAAATACGCAAGAGACTGG |
| | | ATGTGGACATCACGGAACTGCACTCTTGGATTACCAGAAGCGAGGCAGTCCTCCAGAGCCCCGAGT |
| | | TTGCGATCTTCAGGAAGGAAGGCAACTTTTCAGATCTGAAGGAAAAGGTGAACGCCATCGAGCGC |
| | | GAGAAAGCCGAGAAGTTCCGCAAACTGCAGGATGCGAGCCGGTCTGCTCAGGCCTTGGTGGAGCA |
| | | AATGGTGAATGAAGGCGTAAATGCGGACAGCATCAAGCAGGCCTCCGAGCAGCTCAATTCAAGATG |
| | | GATCGAGTTCTGTCAGCTGCTCAGCGAGCGATTGAACTGGCTGGAGTATCAAAATAACATAATCGCG |
| | | TTTTACAACCAGCTGCAACAGCTGGAACAGATGACGACTACTGCAGAGAACTGGCTTAAAATCCAG |
| | | CCTACAACTCCTTCTGAACCCACAGCAATCAAAAGCCAGCTCAAGATCTGCAAGGACGAAGTCAA |
| | | CCGCCTCTCCGATTTGCAGCCTCAGATAGAACGACTCAAGATCCAAAGTATCGCACTCAAAGAAAA |
| | | GGGGCAAGGCCCTATGTTTCTCGATGCTGATTTTGTGGCTTTCACCAACCATTTTAAGCAAGTGTTT |
| | | TCAGATGTGCAAGCCCGGGAAAAGGAACTGCAAACCATTTTTGATACCCTGCCTCCAATGCGGTAC |
| | | CAGGAGACTATGTCTGCAATTCGCACCTGGGTGCAACAAAGCGAGACGAAACTTTCTATTCCACAA |
| | | CTGTCCGTGACAGACTACGAAATTATGGAACAGCGACTGGGAGAACTCCAAGCCCTTCAGTCCAGC |
| | | CTTCAGGAGCAGCAGTCAGGATTGTACTATCTGTCAACCACCGTGAAGGAAATGTCAAAAAAGGCT |
| | | CCCTCCGAAATATCTCGCAAGTATCAGTCTGAGTTTGAAGAGATTGAAGGGAGATGGAAGAAACTG |
| | | TCCTCACAGCTGGTTGAGCACTGTCAGAAGTTGGAAGAGCAGATGAACAAGTTGCGCAAGATTCA |
| | | AAATCACATACAGACACTCAAAAAGTGGATGGCGGAGGTTGACGTCTTCCTTAAAGAGGAGTGGC |
| | | CAGCCCTGGGCGACTCAGAGATCCTGAAAAAGCAACTGAAACAGTGCCGACTGCTGGTGTCCGAC |
| | | ATCCAGACAATACAGCCAAGTTTGAATAGCGTGAATGAGGGTGGACAAAAAATCAAAAACGAGGC |
| | | TGAGCCCGAGTTCGCCTCCAGATTGGAAACCGAGCTGAAGGAACTTAATACACAGTGGGATCATAT |
| | | GTGTCAGCAGGTTTATGCCCGGAAGGAAGCACTGAAAGGGGGGCTGGAGAAAACCGTGAGCCTTC |
| | | AGAAGGACCTTAGCGAGATGCACGAGTGGATGACCCAGGCGGAGGAAGAGTATCTCGAACGGGAT |
| | | TTTGAGTATAAGACACCAGATGAACTTCAGAAGGCAGTCGAAGAGATGAAACGGGCAAAGGAAGA |
| | | GGCTCAGCAGAAGGAAGCAAAGGTTAAACTTCTGACAGAATCCGTTAACAGCGTTATAGCCCAAG |
| | | CTCCCCCCGTTGCCCAGGAAGCTCTTAAAAAGGAACTGGAAACACTGACAACAAACTACCAGTGG |
| | | CTGTGCACCCGCTTGAACGGCAAATGCAAAACCCTGGAAGAGGTCTGGGCTTGTTGGCATGAACT |
| | | CCTGTCATACCTGGAAAAAGCTAATAAGTGGCTGAATGAGGTGGAGTTTAAATTGAAAACGACCGA |
| | | AAACATCCCTGGCGGTGCAGAGGAGATATCTGAGGTGCTCGATAGCCTGGAGAATCTTATGAGGCA |
| | | TAGCGAGGATAACCCCAACCAGATTAGGATACTGGCCCAAACTCTGACCGACGGCGGAGTGATGGA |
| | | CGAGCTGATAAACGAGGAACTGGAAACCTTTAATTCACGCTGGCGAGAACTTCACGAGGAGGCAG |
| | | TCAGGCGCCAGAAACTGCTGGAGCAGTCCATCCAGAGTGCCCAGGAGACTGAGAAGTCACTTCAT |
| | | CTGATCCAGGAGAGTTTGACGTTTATCGACAAGCAGCTTGCCGCCTACATTGCAGACAAGGTCGAT |
| | | GCTGCTCAGATGCCGCAAGAGGCTCAGAAAATCCAGAGCGACCTGACAAGCCACGAGATTAGCCT |
| | | GGAGGAAATGAAAAAGCACAACCAGGGTAAAGAGGCCGCCCAGCGGGTTCTGAGCCAGATCGAT |
| | | GTCGCACAGAAGAAACTCCAAGATGTAAGCATGAAGTTCCGACTGTTTCAGAAACCAGCGAACTT |
| | | TGAGCAGCGACTGCAGGAGTCTAAAATGATCCTCGATGAGGTGAAAATGCATCTGCCAGCCCTGGA |
| | | AACTAAGAGCGTGGAGCAGGAGGTGGTTCAGAGTCAGCTGAACCACTGCGTAAACCTCTATAAAA |
| | | GTCTTAGTGAAGTGAAGAGCGAGGTGGAGATGGTGATTAAGACTGGTAGACAGATTGTACAGAAA |
| | | AAGCAGACGGAGAACCCCAAAGAGTTGGATGAACGGGTGACAGCCCTTAAGCTCCACTATAACGA |
| | | GCTGGGAGCCAAAGTGACTGAGAGAAAGCAGCAACTTGAAAAATGTCTGAAGCTGAGCAGAAAG |
| | | ATGCGGAAGGAAATGAATGTGCTGACAGAGTGGCTGGCTGCGACCGACATGGAACTTACAAAGAG |
| | | GAGCGCCGTGGAAGGCATGCCTTCCAATCTGGATAGTGAGGTCGCTTGGGGCAAAGCTACACAGA |
| | | AGGAAATTGAGAAGCAAAAAGTGCATCTTAAGAGCATTACTGAAGTTGGCGAGGCTTTGAAAACC |
| | | GTCCTCGGCAAGAAAGAGACTCTGGTAGAAGATAAACTCTCACTGCTGAACTCAAACTGGATCGC |
| | | AGTCACTAGTCGGGCCGAAGAGTGGCTGAATTTGCTGTTGGAGTACCAAAAGCATATGGAGACTTT |
| | | CGATCAGAATGTCGACCACATTACGAAGTGGATAATCCAGGCGGACACACTGTTGGATGAGTCCGA |
| | | GAAAAAGAAACCTCAGCAGAAGGAAGACGTGCTCAAACGACTCAAAGCCGAACTGAACGACATC |
| | | AGGCCCAAAGTTGATTCCACCCGGGATCAAGCGGCCAATCTCATGGCCAACCGGGGGGATCACTGC |
| | | AGAAAGTTGGTAGAACCTCAGATAAGCGAACTTAATCATAGATTTGCCGCCATATCACATAGGATTA |
| | | AGACCGGTAAGGCTTCAATCCCCCTGAAGGAACTCGAACAGTTTAATAGTGACATCCAGAAGCTCC |
| | | TGGAGCCTCTCGAAGCCGAGATTCAACAGGGTGTAAACCTTAAGGAAGAGGATTTTAATAAGGATA |
| | | TGAATGAGGACAATGAAGGCACGGTCAAAGAGCTCCTCCAGAGGGGAGACAATTTGCAGCAGCGG |
| | | ATCACCGATGAACGCAAACGCGAAGAGATCAAAATTAAGCAGCAGCTCCTCCAAACAAAGCACAA |
| | | TGCCCTTAAAGACCTCAGAAGCCAGCGGCGGAAAAAGGCTCTGGAAATCAGCCATCAATGGTATCA |
| | | GTACAAGCGGCAAGCGGACGATCTCCTGAAATGTCTGGATGATATCGAGAAAAAACTGGCCAGTCT |
| | | GCCAGAGCCCAGAGATGAGCGCAAAATCAAGGAAATCGACAGGGAGCTTCAGAAGAAAAAAGAG |
| | | GAGCTGAACGCCGTCCGAAGGCAGGCCGAAGGGCTGTCAGAAGATGGGGCCGCCATGGCAGTGG |
| | | AACCTACCCAGATTCAACTGTCCAAGCGCTGGAGAGAGATTGAAAGTAAGTTCGCACAATTCCGGA |
| | | GGCTTAATTTTGCTCAGATTCACACAGTTAGGGAGGAAACCATGATGGTGATGACCGAAGACATGC |
| | | CACTCGAAATCAGCTACGTCCCTAGCACATATCTCACGGAGATTACCCACGTGTCCCAGGCACTGCT |
| | | GGAAGTCGAGCAATTGTTGAACGCCCCTGATCTCTGCGCCAAGGATTTCGAAGACTTGTTCAAGCA |
| | | AGAGGAAAGCCTCAAGAACATCAAAGACAGTCTTCAGCAGTCCTCAGGCAGAATTGATATTATCCA |
| | | CTCCAAGAAAACAGCCGCTCTCCAGAGCGCTACCCCTGTCGAACGAGTGAAACTGCAAGAGGCCT |
| | | TGTCTCAGTTGGACTTCCAGTGGGAGAAAGTCAACAAGATGTACAAGGACAGGCAGGGTCGGTTC |
| | | GACCGGAGCGTCGAAAAATGGCGGAGATTCCATTATGACATCAAGATATTCAATCAGTGGCTTACAG |
| | | AGGCGGAGCAATTTTTGAGAAAGACGCAGATTCCCGAAAACTGGGAGCATGCCAAGTACAAGTGG |
| | | TACCTCAAAGAGCTCCAGGATGGAATCGGCCAGAGACAGACCGTAGTTAGAACACTGAACGCAAC |
| | | TGGAGAAGAGATTATCCAGCAGTCCTCCAAAACTGACGCCTCTATCCTCCAGGAGAAGCTTGGCAG |
| | | CCTTAACCTGCGGTGGCAGGAGGTGTGTAAACAACTGTCAGATAGGAAAAAAAGACTTGAGGAAC |
| | | AGAAGAACATTCTGAGCGAGTTCCAACGAGATCTCAACGAGTTCGTACTGTGGCTGGAGGAGGCC |
| | | GACAACATCGCTTCAATCCCCCTGGAACCCGGCAAGGAACAGCAGCTTAAGGAAAAATTGGAACA |
| | | GGTAAAACTGTTGGTAGAGGAGCTCCCATTGCGGCAGGGCATTCTCAAGCAGCTGAATGAAACTGG |
| | | AGGTCCCGTTCTGGTCAGTGCGCCCATAAGCCCGGAAGAGCAGGACAAGTTGGAAAACAAGCTGA |
| | | AGCAAACAAATCTGCAGTGGATCAAAGTGAGCCGGGCTCTTCCAGAAAAGCAAGGCGAGATTGAA |
| | | GCCCAGATTAAGGATCTTGGCCAGCTTGAGAAAAAGCTGGAGGATCTGGAAGAGCAGCTGAACCA |
| | | TTTGCTTCTGTGGCTGAGTCCCATTAGAAACCAGCTGGAAATCTACAACCAGCCAAACCAAGAGGG |
| | | CCCTTTCGACGTCAAGGAAACTGAGATAGCAGTCCAGGCAAAACAGCCCGATGTCGAAGAAATCT |
| | | TGTCTAAGGGGCAGCACCTCTACAAAGAGAAACCTGCCACCCAGCCCGTGAAGAGAAAACTGGAA |
| | | GACCTCTCTTCAGAGTGGAAGGCTGTAAACAGGTTGCTCCAGGAGCTGAGAGCTAAGCAACCTGA |
| | | CCTCGCACCCGGCCTGACTACTATTGGCGCCAGCCCGACCCAAACCGTCACACTGGTGACCCAGCC |
| | | TGTAGTCACAAAAGAAACCGCCATCTCCAAACTGGAGATGCCTTCAAGCCTGATGCTGGAGGTGCC |
| | | TGCTCTGGCCGATTTCAACAGAGCCTGGACAGAGCTGACAGACTGGCTGTCTCTGCTCGACCAAGT |
| | | GATAAAGTCACAGCGCGTCATGGTGGGTGACCTGGAGGACATCAATGAGATGATCATAAAGCAAAA |
| | | GGCTACCATGCAGGACTTGGAGCAGCGCAGGCCGCAGTTGGAAGAACTGATTACAGCAGCCCAGA |
| | | ACCTCAAGAACAAGACTTCAAATCAGGAGGCAAGGACCATCATAACAGATAGAATCGAGAGGATT |
| | | CAGAACCAATGGGATGAGGTGCAGGAGCACCTGCAGAACAGAAGGCAGCAGCTGAACGAAATGC |
| | | TGAAAGACTCCACACAGTGGCTGGAGGCCAAAGAGGAGGCAGAACAGGTGCTGGGCCAGGCTAG |
| | | GGCCAAGCTGGAAAGTTGGAAGGAAGGGCCCTACACCGTGGACGCCATACAGAAGAAAATAACGG |
| | | AGACAAAACAGCTGGCCAAAGACCTCCGCCAATGGCAGACCAACGTAGACGTCGCCAATGACCTG |
| | | GCACTCAAGTTGTTGAGAGATTATTCCGCAGATGACACCCGAAAAGTACACATGATCACTGAGAAT |
| | | ATCAACGCCAGTTGGCGGAGCATTCACAAACGGGTGAGCGAGAGAGAAGCCGCCCTTGAGGAGA |
| | | CGCATCGCCTTTTGCAGCAATTTCCGCTTGACCTGGAAAAGTTTCTGGCTTGGTTGACAGAGGCCG |
| | | AGACAACTGCTAACGTGCTGCAAGATGCCACCCGCAAAGAGCGGCTTCTTGAGGACTCTAAAGGG |
| | | GTGAAAGAATTGATGAAGCAGTGGCAGGACCTCCAGGGCGAAATCGAGGCCCATACTGATGTCTAC |
| | | CACAACCTGGATGAAAATAGTCAGAAAATCCTGAGATCCCTGGAAGGGAGTGATGATGCGGTGCTG |
| | | CTGCAGCGCCGACTCGATAACATGAACTTTAAGTGGTCTGAACTCCGGAAAAAAAGCCTGAACATT |
| | | CGGAGCCATCTGGAGGCTTCTTCTGATCAGTGGAAGAGACTTCATCTGTCCTTGCAGGAGCTTCTG |
| | | GTGTGGCTGCAGCTGAAAGACGACGAGCTGTCAAGGCAGGCACCAATAGGGGGCGACTTCCCCGC |
| | | TGTTCAGAAACAAAATGATGTGCACAGGGCATTCAAGCGGGAACTGAAGACGAAAGAACCAGTGA |
| | | TTATGAGCACTCTGGAGACGGTACGCATCTTTCTGACCGAGCAGCCACTTGAAGGCCTCGAAAAGT |
| | | TGTACCAGGAGCCACGGGAATTGCCTCCCGAAGAACGGGCTCAGAATGTTACCCGACTGCTTCGCA |
| | | AGCAGGCTGAGGAAGTGAACACTGAGTGGGAAAAACTGAATCTGCACAGTGCCGATTGGCAGCGC |
| | | AAAATTGACGAGACATTGGAGCGCTTGCGGGAGCTGCAGGAAGCCACAGATGAACTTGATCTGAA |
| | | ACTCAGACAGGCCGAGGTCATAAAGGGTTCTTGGCAACCCGTAGGCGACCTTTTGATTGACTCACT |
| | | GCAGGATCATCTGGAGAAAGTAAAGGCGCTGCGGGGGGAAATTGCTCCGCTGAAGGAAAACGTGA |
| | | GCCATGTGAATGATCTGGCTCGGCAATTGACTACCCTGGGTATCCAGCTGTCCCCCTACAATCTGAG |
| | | CACTCTCGAAGACCTCAACACAAGATGGAAACTGCTGCAGGTCGCCGTGGAGGACAGGGTGAGGC |
| | | AGCTGCATGAGGCACACAGGGACTTCGGTCCGGCAAGCCAACACTTCCTGTCTACTTCTGTTCAAG |
| | | GGCCTTGGGAACGCGCTATTTCCCCAAACAAAGTGCCGTACTATATTAACCATGAAACTCAGACAAC |
| | | CTGCTGGGATCATCCTAAAATGACTGAGCTGTACCAGAGCCTCGCCGATCTGAATAACGTGAGGTTT |
| | | AGCGCATACAGAACCGCAATGAAGCTCAGGAGACTGCAGAAGGCACTTTGCCTGGACCTCCTGAG |
| | | CCTCTCCGCTGCCTGCGATGCCCTCGACCAGCACAACCTGAAGCAGAATGATCAGCCTATGGACAT |
| | | CCTGCAAATCATAAATTGTCTGACGACAATCTACGACCGGCTCGAACAGGAGCACAATAATCTCGTG |
| | | AACGTTCCACTTTGTGTGGATATGTGTCTCAACTGGCTGTTGAACGTCTATGACACCGGGAGAACTG |
| | | GAAGGATCCGCGTTCTGTCCTTTAAGACTGGAATCATCAGCTTGTGTAAGGCGCACCTTGAGGACA |
| | | AATATCGATACCTGTTTAAGCAGGTTGCAAGCTCCACTGGATTCTGCGACCAGAGGCGCCTCGGGCT |
| | | GCTGCTTCATGATAGTATCCAAATCCCACGCCAACTCGGAGAGGTGGCAAGCTTCGGCGGGAGCAA |
| | | TATTGAGCCAAGCGTCAGATCTTGTTTCCAGTTTGCCAACAATAAGCCTGAGATCGAAGCCGCACT |
| | | GTTTTTGGATTGGATGCGGTTGGAGCCCCAGAGTATGGTGTGGTTGCCCGTGCTGCATAGGGTCGCT |
| | | GCCGCTGAAACCGCCAAACACCAGGCCAAGTGCAATATTTGCAAGGAATGTCCCATCATCGGGTTT |
| | | AGATACCGGAGCCTGAAGCACTTCAATTATGATATCTGCCAGTCTTGTTTTTTTTCCGGCCGAGTTGC |
| | | TAAGGGACACAAGATGCATTACCCAATGGTGGAATATTGTACGCCAACCACCAGCGGCGAAGATGT |
| | | CAGAGATTTTGCAAAAGTACTCAAGAACAAGTTCCGCACCAAGCGATATTTCGCGAAGCACCCCCG |
| | | GATGGGCTACCTCCCAGTGCAGACGGTTCTGGAAGGCGATAACATGGAAACTCCAGTCACCCTGAT |
| | | CAATTTCTGGCCTGTGGACTCTGCGCCTGCCTCTTCACCTCAGCTCTCCCACGATGACACCCATTCC |
| | | AGAATCGAGCACTACGCGTCTCGACTGGCGGAGATGGAGAACAGCAATGGTTCTTACCTCAATGAC |
| | | AGCATTAGCCCCAACGAAAGCATCGATGACGAGCACCTCTTGATTCAACACTACTGCCAGTCCCTG |
| | | AATCAGGACAGTCCTCTGTCCCAACCCAGGAGCCCAGCCCAGATACTGATCTCTCTCGAATCAGAA |
| | | GAAAGAGGGGAGTTGGAGCGAATTTTGGCCGACCTGGAGGAGGAGAATAGAAATCTTCAGGCCGA |
| | | GTACGATAGACTGAAGCAGCAGCACGAGCACAAGGGCCTTTCTCCACTGCCCTCTCCACCGGAAAT |
| | | GATGCCAACCTCCCCTCAAAGTCCCAGGGACGCTGAGCTCATTGCCGAGGCAAAGCTGTTGCGCC |
| | | AGCACAAAGGACGCCTGGAGGCGAGAATGCAGATCCTGGAAGACCATAACAAGCAGCTTGAGTCT |
| | | CAGCTGCACCGACTTAGACAGCTGTTGGAGCAGCCACAAGCGGAAGCAAAGGTCAATGGAACTAC |
| | | CGTGAGTTCCCCCAGTACAAGTCTGCAGCGCTCTGACTCAAGCCAGCCAATGCTGCTCCGGGTAGT |
| | | CGGTTCTCAGACTAGTGATTCAATGGGGGAGGAGGATCTTCTTTCCCCACCCCAAGACACCTCAAC |
| | | AGGGCTTGAGGAAGTGATGGAGCAGCTGAACAACTCCTTCCCCTCTTCACGGGGGAGAAATACTC |
| | | CTGGGAAGCCTATGAGAGAGGATACCATGTAATGA |
| 21. | CBA-hBGi- | CGTTACATAACTTACGGTAAATGGCCCGCCTGGCTGACCGCCCAACGACCCCCGCCCATTGACGTC |
| | DMD-1- | AATAATGACGTATGTTCCCATAGTAACGCCAATAGGGACTTTCCATTGACGTCAATGGGTGGAGTATT |
| | WPRE3- | TACGGTAAACTGCCCACTTGGCAGTACATCAAGTGTATCATATGCCAAGTACGCCCCCTATTGACGT |
| | DTS | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | CATGCTGTGGTGGGAAGAGGTGGAGGATTGCTACGAGCGGGAGGATGTGCAGAAAAAGACTTTCA |
| | | CCAAGTGGGTAAATGCTCAGTTCTCTAAGTTCGGGAAGCAACACATCGAGAACTTGTTTTCCGACC |
| | | TCCAGGATGGCAGGCGCCTGCTTGACTTGCTGGAGGGTCTGACTGGTCAGAAGCTCCCTAAGGAG |
| | | AAGGGCAGCACCAGAGTTCACGCTCTGAATAACGTTAACAAAGCCCTGAGGGTCCTCCAGAATAA |
| | | CAATGTAGACCTGGTGAACATCGGTTCTACCGACATCGTGGACGGGAATCATAAACTGACCTTGGG |
| | | GCTCATCTGGAACATTATCTTGCACTGGCAGGTGAAGAACGTCATGAAGAATATCATGGCCGGGCTG |
| | | CAACAGACCAACTCCGAAAAGATTTTGCTCAGCTGGGTGCGCCAGTCCACCCGCAATTATCCACAA |
| | | GTCAACGTTATCAATTTCACCACTAGTTGGAGTGATGGCCTCGCCCTGAACGCCTTGATCCACAGTC |
| | | ACCGCCCCGACCTGTTTGACTGGAACTCTGTGGTTTGCCAACAGAGCGCTACCCAGCGTCTCGAAC |
| | | ATGCTTTCAATATCGCTAGATACCAGCTCGGTATCGAGAAGCTGTTGGATCCTGAGGACGTAGACAC |
| | | GACCTACCCCGATAAAAAGAGCATCCTGATGTATATCACTTCTCTGTTTCAGGTGCTTCCTCAACAG |
| | | GTGTCCATTGAAGCCATCCAAGAGGTCGAAATGCTGCCCAGGCCCCCTAAGGTGACAAAAGAAGA |
| | | GCATTTCCAGCTGCATCACCAGATGCATTACAGTCAACAGATTACCGTCAGCTTGGCTCAGGGATAT |
| | | GAGCGCACGTCCTCTCCTAAGCCCAGATTTAAGTCCTACGCATATACCCAGGCTGCCTACGTGACCA |
| | | CTTCCGACCCAACTAGGAGCCCCTTTCCCTCCCAACACCTGGAGGCCCCTGAGGACAAAAGCTTCG |
| | | GATCTAGTCTGATGGAGTCCGAGGTGAATCTGGATCGGTACCAAACTGCTCTGGAGGAAGTGCTGT |
| | | CCTGGCTTCTGTCCGCTGAAGACACCCTGCAGGCGCAAGGTGAGATTTCTAACGACGTCGAGGTCG |
| | | TGAAGGATCAGTTCCACACTCATGAGGGCTACATGATGGACTTGACAGCTCACCAGGGAAGAGTTG |
| | | GTAACATCCTGCAGCTTGGCTCCAAGTTGATTGGCACGGGAAAGCTTAGCGAGGACGAGGAAACC |
| | | GAGGTACAGGAGCAGATGAACTTGCTGAATTCCCGCTGGGAGTGCCTGAGAGTTGCGTCCATGGA |
| | | AAAGCAGTCAAACCTTCACAGAGTGCTGATGGACCTTCAGAACCAGAAGCTGAAGGAGCTGAACG |
| | | ATTGGCTGACAAAGACCGAGGAACGCACAAGAAAAATGGAAGAGGAACCTCTGGGACCTGATCTG |
| | | GAGGATCTGAAGCGCCAGGTCCAACAGCATAAGGTGCTGCAGGAGGATTTGGAGCAGGAGCAGGT |
| | | GCGGGTCAACAGCCTGACACATATGGTGGTTGTGGTCGATGAGTCCTCTGGCGATCATGCTACCGC |
| | | AGCGCTGGAAGAGCAGCTGAAGGTGCTGGGGGATCGCTGGGCCAACATTTGCAGATGGACAGAAG |
| | | ACCGGTGGGTCCTGCTTCAAGATATCCTGCTCAAGTGGCAGCGCCTGACTGAAGAGCAGTGCCTGT |
| | | TCTCAGCTTGGTTGAGCGAAAAAGAGGACGCAGTTAACAAGATCCATACCACAGGCTTCAAGGATC |
| | | AGAATGAGATGCTGAGCTCACTGCAGAAGCTGGCTGTGCTGAAGGCCGACTTGGAGAAGAAAAAG |
| | | CAGTCCATGGGCAAACTGTATAGCCTGAAGCAGGACCTGCTCAGCACCCTGAAGAACAAAAGCGT |
| | | TACGCAGAAAACTGAGGCCTGGTTGGACAACTTTGCTCGCTGCTGGGATAACCTTGTGCAGAAGCT |
| | | GGAAAAGTCAACCGCACAGATCTCCCAGGCTGTGACCACTACGCAGCCTAGCTTGACCCAGACAA |
| | | CCGTTATGGAAACCGTGACAACCGTGACAACGAGAGAGCAAATCCTGGTTAAGCATGCCCAGGAG |
| | | GAACTGCCGCCACCCCCGCCTCAGAAGAAACGGCAGATTACGGTGGACAGCGAGATCCGCAAGAG |
| | | GCTGGACGTCGACATCACTGAGCTCCACTCATGGATCACGCGGAGTGAGGCCGTGCTGCAGTCTCC |
| | | CGAGTTCGCTATTTTCCGCAAGGAGGGAAACTTCTCTGACTTGAAAGAGAAGGTGAATGCCATCGA |
| | | GCGTGAAAAGGCAGAAAAGTTCAGAAAGCTGCAGGACGCCTCTCGCTCTGCGCAGGCCCTGGTGG |
| | | AGCAGATGGTCAACGAGGGTGTTAACGCTGACAGCATCAAACAGGCAAGCGAACAGCTGAACTCC |
| | | AGATGGATCGAATTTTGCCAACTGCTCTCCGAACGCCTCAACTGGTTGGAGTACCAGAACAATATTA |
| | | TCGCTTTTTACAACCAGCTGCAGCAACTGGAGCAGATGACAACTACCGCCGAAAACTGGCTGAAA |
| | | ATTCAGCCAACCACACCGTCCGAGCCAACTGCGATCAAGTCTCAGCTGAAGATCTGCAAGGACGA |
| | | AGTGAACCGTCTTAGCGACTTGCAACCCCAGATTGAACGGCTCAAGATTCAGTCTATTGCTCTGAA |
| | | GGAGAAGGGTCAGGGTCCGATGTTCCTGGATGCAGATTTTGTCGCATTTACCAATCACTTCAAACA |
| | | GGTGTTTAGTGACGTCCAGGCGCGCGAGAAAGAGCTGCAGACTATTTTTGACACCCTGCCTCCCAT |
| | | GAGGTACCAGGAGACAATGAGTGCCATTCGCACCTGGGTGCAACAGTCCGAGACGAAATTGTCAA |
| | | TCCCACAGCTGTCCGTGACAGACTACGAGATTATGGAGCAACGGTTGGGGGAGCTGCAGGCTCTTC |
| | | AGAGTTCCCTGCAGGAGCAACAGAGCGGACTGTACTATCTTTCCACTACCGTGAAGGAAATGAGTA |
| | | AGAAAGCCCCTTCCGAGATCAGCCGCAAATACCAGTCTGAATTCGAAGAGATCGAGGGACGCTGG |
| | | AAAAAGCTGTCAAGCCAGCTGGTCGAGCACTGTCAGAAGCTGGAAGAGCAGATGAACAAGCTGC |
| | | GTAAAATCCAGAACCATATTCAGACCCTCAAGAAATGGATGGCAGAAGTTGACGTGTTTCTGAAGG |
| | | AAGAGTGGCCTGCCCTCGGCGATAGCGAAATCCTTAAGAAACAACTCAAGCAGTGCCGCCTCCTG |
| | | GTGTCCGATATTCAGACAATCCAGCCTTCCCTGAACAGCGTTAATGAGGGCGGACAGAAAATTAAA |
| | | AACGAGGCCGAACCCGAGTTCGCTTCTCGCCTGGAGACTGAACTGAAGGAGCTGAATACCCAGTG |
| | | GGACCATATGTGTCAACAGGTCTACGCACGCAAAGAGGCACTGAAGGGCGGTCTGGAGAAGACTG |
| | | TCTCACTCCAGAAGGACCTGAGCGAGATGCACGAGTGGATGACCCAGGCCGAAGAGGAATACCTG |
| | | GAGCGTGATTTTGAGTATAAGACCCCAGACGAACTCCAGAAAGCCGTTGAAGAGATGAAGAGGGC |
| | | TAAGGAGGAAGCCCAACAGAAGGAGGCGAAAGTGAAGCTCCTGACAGAGTCCGTGAACTCCGTC |
| | | ATCGCTCAAGCTCCTCCCGTGGCTCAGGAGGCCCTCAAGAAAGAGCTGGAAACTCTGACCACAAA |
| | | TTACCAGTGGTTGTGTACCCGCTTGAACGGTAAGTGCAAGACCCTGGAAGAGGTGTGGGCCTGCTG |
| | | GCACGAGCTCCTGTCCTATCTTGAGAAGGCGAACAAATGGCTGAACGAAGTGGAATTCAAGTTGA |
| | | AAACAACTGAGAACATTCCGGGAGGCGCTGAGGAAATCTCTGAGGTGCTGGACTCTCTGGAAAAT |
| | | CTGATGCGTCATTCTGAAGACAATCCTAACCAGATCAGGATTCTGGCACAGACCCTCACTGACGGA |
| | | GGCGTTATGGACGAACTTATCAACGAGGAACTGGAGACATTCAATTCCCGTTGGCGTGAGCTGCAC |
| | | GAAGAGGCCGTGCGCCGTCAGAAGTTGCTGGAACAGTCTATCCAGTCCGCCCAGGAGACAGAGAA |
| | | ATCTCTCCACCTGATCCAGGAGAGTCTGACGTTTATCGATAAGCAGCTGGCTGCCTACATCGCCGAC |
| | | AAGGTGGACGCTGCCCAGATGCCCCAAGAGGCTCAGAAGATCCAGTCCGACCTGACGTCTCATGA |
| | | AATCAGCCTGGAAGAGATGAAAAAGCACAACCAGGGCAAAGAGGCTGCCCAGCGCGTCCTCTCCC |
| | | AGATTGACGTGGCTCAGAAAAAGTTGCAGGACGTGAGCATGAAGTTTAGGCTGTTCCAGAAACCC |
| | | GCCAACTTCGAACAGAGACTGCAGGAGTCTAAGATGATCCTGGATGAGGTCAAGATGCACCTCCCT |
| | | GCTCTGGAAACAAAATCTGTGGAGCAGGAGGTCGTGCAGTCACAACTGAACCATTGTGTGAACCT |
| | | CTACAAGTCTCTCAGCGAGGTGAAGTCCGAGGTCGAGATGGTGATCAAGACCGGACGCCAGATTG |
| | | TGCAGAAAAAGCAGACAGAGAACCCAAAGGAGCTGGACGAACGGGTGACCGCCCTGAAGCTCCA |
| | | CTACAACGAGTTGGGTGCCAAAGTCACTGAAAGAAAGCAACAGCTGGAGAAGTGTCTGAAGCTG |
| | | AGCCGCAAGATGCGCAAAGAAATGAACGTCTTGACCGAGTGGTTGGCGGCCACCGACATGGAGCT |
| | | GACCAAGAGATCCGCTGTGGAGGGCATGCCCTCCAACCTTGACTCAGAGGTGGCCTGGGGCAAGG |
| | | CCACACAGAAGGAGATCGAAAAACAGAAGGTGCATCTTAAATCAATCACTGAGGTGGGCGAAGCT |
| | | CTGAAGACAGTGCTGGGCAAGAAAGAGACACTTGTAGAGGACAAGCTCTCCCTTCTGAATAGCAA |
| | | TTGGATCGCCGTAACTAGTCGCGCGGAGGAATGGCTGAACCTCCTGTTGGAATACCAGAAACACAT |
| | | GGAAACCTTCGATCAGAACGTGGACCATATTACCAAGTGGATTATCCAGGCTGACACTCTCCTGGAC |
| | | GAGTCTGAGAAAAAGAAACCCCAGCAAAAGGAAGATGTGCTGAAGCGCCTGAAGGCGGAATTGA |
| | | ACGACATCCGCCCGAAGGTTGATTCCACCCGCGACCAAGCGGCCAACCTTATGGCTAACCGCGGGG |
| | | ACCATTGCAGAAAACTGGTGGAACCGCAGATCTCTGAGCTGAACCACAGATTCGCCGCGATTTCTC |
| | | ACAGAATTAAGACTGGCAAGGCCTCCATCCCTCTGAAAGAGCTGGAGCAGTTCAACTCTGATATTC |
| | | AGAAGCTTCTGGAGCCCCTGGAGGCAGAAATCCAACAGGGCGTCAACCTGAAAGAGGAAGACTTT |
| | | AATAAGGACATGAATGAGGATAACGAAGGTACCGTCAAGGAGCTCCTGCAGAGGGGCGACAATCT |
| | | GCAGCAAAGAATCACTGACGAACGTAAGCGCGAAGAGATTAAGATCAAGCAACAGCTGTTGCAGA |
| | | CTAAGCACAACGCCCTGAAGGATCTGAGGTCCCAGCGCAGGAAAAAGGCCCTGGAGATTTCCCAC |
| | | CAGTGGTACCAGTACAAGCGCCAGGCCGATGACCTTCTGAAGTGCCTCGACGATATCGAGAAAAAG |
| | | CTGGCCTCACTGCCCGAGCCCCGCGACGAGCGCAAGATCAAGGAGATTGACCGTGAGCTCCAGAA |
| | | GAAAAAGGAGGAACTGAACGCGGTGCGCAGACAGGCCGAGGGCCTCTCCGAGGACGGCGCTGCG |
| | | ATGGCTGTTGAGCCCACTCAGATCCAACTGAGCAAGCGGTGGAGAGAGATCGAGTCTAAGTTCGC |
| | | GCAATTTAGGCGCCTCAACTTCGCCCAGATTCACACGGTCCGTGAGGAAACGATGATGGTGATGAC |
| | | AGAGGATATGCCACTGGAAATCAGCTACGTGCCCAGTACATATCTCACCGAAATCACCCATGTGTCC |
| | | CAGGCTCTCCTGGAGGTGGAACAGTTGCTCAACGCTCCCGACTTGTGCGCTAAGGACTTCGAGGA |
| | | CCTGTTCAAGCAGGAAGAGAGCCTGAAAAACATTAAGGACTCCCTGCAACAGTCAAGCGGCCGCA |
| | | TCGATATCATTCATTCTAAGAAAACCGCTGCCCTGCAGTCTGCAACCCCCGTAGAGCGCGTCAAACT |
| | | GCAGGAAGCTCTCAGCCAACTGGATTTCCAGTGGGAGAAGGTGAACAAGATGTATAAGGATCGTC |
| | | AGGGCCGGTTCGACCGCTCTGTGGAGAAGTGGCGCCGTTTCCACTACGATATCAAGATTTTCAATC |
| | | AATGGTTGACCGAGGCAGAGCAGTTCCTCAGAAAGACACAGATCCCTGAAAATTGGGAGCATGCC |
| | | AAGTACAAGTGGTATCTGAAGGAGCTGCAGGACGGGATCGGACAACGTCAGACCGTGGTCCGCAC |
| | | CCTGAACGCAACCGGCGAGGAAATTATCCAACAGTCTAGCAAGACAGATGCTAGCATCCTCCAGGA |
| | | GAAGCTCGGAAGCCTCAACCTTCGCTGGCAGGAGGTGTGCAAGCAGCTGTCAGACAGGAAGAAA |
| | | CGCCTGGAGGAACAGAAGAACATCCTTTCAGAGTTCCAGAGAGATCTGAACGAATTCGTGCTTTGG |
| | | CTTGAAGAGGCCGATAACATCGCAAGCATCCCACTGGAGCCTGGTAAGGAACAGCAATTGAAGGA |
| | | GAAGCTGGAGCAGGTAAAGCTGCTCGTGGAAGAGCTGCCTCTGAGGCAGGGAATTCTCAAGCAGC |
| | | TCAACGAGACTGGGGGCCCGGTTTTGGTGAGCGCCCCAATTAGCCCAGAAGAGCAGGACAAGTTG |
| | | GAGAACAAGCTGAAGCAGACCAACCTGCAGTGGATCAAGGTGTCCCGCGCACTGCCCGAAAAGC |
| | | AGGGAGAGATTGAGGCCCAGATCAAGGATCTGGGACAGCTGGAGAAAAAGCTGGAAGACCTGGA |
| | | GGAACAGCTGAACCATTTGCTCCTGTGGTTGTCCCCCATCAGAAATCAGCTGGAAATCTACAACCA |
| | | ACCGAACCAGGAAGGTCCGTTTGATGTTAAGGAAACCGAGATCGCTGTGCAGGCGAAGCAGCCTG |
| | | ACGTAGAAGAGATCCTGTCAAAGGGACAGCACCTCTACAAGGAGAAGCCCGCCACCCAGCCAGTC |
| | | AAGCGGAAGCTCGAAGACCTGTCCAGCGAGTGGAAAGCCGTCAACCGCCTCCTGCAAGAGCTGC |
| | | GTGCCAAGCAACCGGACCTGGCGCCAGGTCTGACTACCATTGGCGCAAGTCCTACCCAGACTGTCA |
| | | CCCTGGTGACCCAACCCGTTGTGACAAAGGAAACCGCCATCAGCAAGCTGGAGATGCCTAGCTCTT |
| | | TGATGCTGGAGGTTCCCGCCCTCGCCGACTTCAACCGTGCTTGGACAGAACTGACCGACTGGCTGT |
| | | CCCTCCTGGACCAAGTGATCAAGAGCCAGCGCGTGATGGTGGGTGATCTTGAGGATATTAACGAAA |
| | | TGATTATCAAGCAGAAGGCCACCATGCAGGACTTGGAGCAGAGGCGCCCCCAGTTGGAGGAACTG |
| | | ATCACCGCCGCGCAGAATCTGAAAAACAAAACTTCCAATCAGGAAGCGAGAACCATCATTACAGA |
| | | CAGAATTGAGAGAATTCAAAACCAGTGGGACGAAGTGCAAGAACACCTCCAGAACCGGCGTCAA |
| | | CAGCTGAACGAGATGCTGAAGGATTCCACTCAATGGCTGGAAGCTAAGGAAGAGGCCGAGCAGGT |
| | | CCTTGGACAGGCACGCGCTAAGCTGGAGTCCTGGAAAGAAGGCCCCTATACAGTGGACGCAATTC |
| | | AAAAGAAAATCACCGAGACGAAGCAACTGGCCAAGGACCTGAGACAGTGGCAAACCAACGTTGA |
| | | TGTGGCTAATGACCTGGCCTTGAAGCTCCTGCGTGACTACTCCGCGGATGACACTAGAAAGGTGCA |
| | | CATGATCACCGAGAATATCAACGCTAGCTGGCGCTCCATCCATAAGCGTGTGAGTGAGCGCGAAGC |
| | | CGCTCTGGAGGAAACCCATCGCCTGCTTCAACAGTTTCCTTTGGACCTGGAAAAGTTCCTGGCCTG |
| | | GCTCACAGAAGCCGAGACAACCGCCAACGTGCTGCAGGACGCCACCCGTAAGGAGCGCTTGCTGG |
| | | AGGATAGTAAGGGGGTGAAGGAGTTGATGAAGCAGTGGCAAGATCTGCAGGGGGAAATCGAGGCA |
| | | CATACCGACGTCTATCATAACCTGGATGAGAATTCCCAGAAAATCCTGCGCAGCTTGGAGGGCAGT |
| | | GATGACGCGGTGCTTCTGCAACGGCGCTTGGACAACATGAACTTCAAGTGGTCCGAGTTGCGTAAA |
| | | AAGAGTCTGAACATTCGCTCCCACCTGGAGGCTTCTAGTGACCAGTGGAAGCGCCTGCACCTGAGC |
| | | CTGCAAGAACTGCTCGTGTGGCTGCAACTGAAGGATGACGAGTTGAGTCGTCAGGCCCCTATCGGA |
| | | GGTGACTTTCCGGCTGTCCAGAAACAGAACGACGTGCATAGGGCATTTAAGCGCGAGTTGAAGAC |
| | | AAAGGAGCCAGTCATTATGTCCACGCTGGAAACCGTGCGCATTTTCCTGACTGAGCAGCCTCTGGA |
| | | GGGGCTGGAGAAGCTGTACCAGGAGCCTCGCGAGTTGCCCCCTGAAGAGCGTGCCCAGAATGTGA |
| | | CCCGCCTGTTGCGTAAGCAGGCCGAGGAAGTGAACACCGAGTGGGAGAAGCTGAACCTGCATTCC |
| | | GCGGATTGGCAGAGGAAGATTGACGAGACGCTGGAGAGGCTCCGTGAGTTGCAGGAGGCAACCG |
| | | ACGAACTGGACCTCAAGCTGCGTCAAGCAGAGGTGATCAAGGGGAGTTGGCAGCCGGTCGGCGAT |
| | | TTGCTGATCGACTCCCTGCAGGATCACCTGGAAAAGGTTAAAGCTTTGCGCGGGGAAATTGCCCCG |
| | | CTGAAGGAGAACGTGAGCCACGTTAACGATCTGGCCAGACAGCTGACCACTCTGGGTATCCAGCT |
| | | GAGCCCGTACAATCTGAGCACCCTCGAAGACTTGAATACGAGATGGAAACTCCTGCAGGTGGCAGT |
| | | GGAGGACAGGGTGCGCCAATTGCACGAGGCCCACCGGGACTTTGGACCGGCCAGCCAGCATTTCC |
| | | TCAGTACCTCTGTTCAGGGCCCGTGGGAGCGGGCCATCTCCCCCAACAAGGTCCCCTATTACATCAA |
| | | CCATGAAACCCAGACTACCTGTTGGGACCACCCTAAAATGACTGAGTTGTATCAATCTCTGGCCGAC |
| | | TTGAATAACGTGCGCTTCTCAGCCTACAGGACTGCAATGAAACTCAGACGCCTCCAAAAGGCCCTT |
| | | TGTCTGGACTTGCTGTCCCTTAGCGCTGCCTGTGATGCTCTGGATCAGCACAATTTGAAACAAAACG |
| | | ACCAGCCGATGGACATCCTCCAGATTATCAACTGCCTGACTACCATCTACGACAGACTGGAACAGG |
| | | AGCATAATAACCTGGTGAACGTCCCCCTGTGCGTGGACATGTGCCTGAACTGGCTGCTCAATGTGTA |
| | | TGACACGGGTAGAACAGGACGGATCAGGGTCCTGTCCTTTAAGACCGGTATCATTTCTCTGTGTAA |
| | | GGCACATCTGGAGGACAAGTACCGCTACCTGTTTAAGCAGGTCGCCAGCTCCACTGGCTTCTGCGA |
| | | TCAAAGGCGGCTGGGTCTGCTCCTGCACGACTCCATTCAGATCCCGAGACAGTTGGGTGAAGTGGC |
| | | GTCCTTCGGCGGTTCTAATATCGAGCCTAGCGTCCGCAGCTGTTTCCAATTCGCCAATAACAAGCCG |
| | | GAGATCGAAGCGGCCCTTTTCCTGGACTGGATGCGTCTGGAACCTCAGAGCATGGTTTGGCTTCCG |
| | | GTGCTTCATCGCGTGGCCGCTGCCGAAACCGCTAAACATCAGGCCAAATGTAACATCTGTAAGGAA |
| | | TGCCCGATTATCGGTTTCCGGTACCGTAGCCTCAAGCATTTCAACTATGATATTTGTCAATCTTGCTTC |
| | | TTTAGCGGACGGGTGGCCAAAGGCCACAAAATGCACTACCCGATGGTCGAGTATTGCACTCCTACA |
| | | ACCTCCGGCGAGGACGTGCGCGATTTTGCCAAAGTGCTGAAAAACAAGTTTAGAACCAAGCGTTA |
| | | CTTTGCCAAGCATCCACGTATGGGCTACCTGCCTGTCCAGACCGTTCTGGAAGGTGATAACATGGAG |
| | | ACACCCGTGACTTTGATTAACTTTTGGCCAGTAGACTCTGCTCCGGCAAGCTCCCCTCAGCTCTCCC |
| | | ACGATGACACTCACAGCCGTATCGAGCACTACGCCTCTCGCCTGGCTGAGATGGAGAACTCTAACG |
| | | GTTCATACCTGAATGACAGTATCTCCCCAAACGAGAGTATCGATGACGAACATCTGCTCATTCAGCA |
| | | TTACTGTCAATCCTTGAATCAGGACAGCCCCCTGTCCCAGCCACGGTCCCCGGCCCAGATCCTGATT |
| | | TCACTGGAGTCCGAAGAGAGAGGGGAACTGGAAAGGATCCTCGCTGACCTGGAAGAGGAAAACC |
| | | GGAACCTGCAGGCTGAATACGACCGCTTGAAGCAGCAACACGAGCACAAGGGCTTGAGCCCGTTG |
| | | CCAAGCCCTCCGGAGATGATGCCAACTAGCCCTCAGAGCCCACGGGATGCCGAGCTCATCGCTGAA |
| | | GCTAAGTTGCTGAGACAGCACAAGGGACGTCTGGAGGCCCGCATGCAGATCCTGGAGGATCATAAT |
| | | AAACAGTTGGAGAGCCAACTGCATAGGCTGCGCCAGCTCCTGGAGCAGCCACAGGCCGAGGCTAA |
| | | GGTCAACGGAACTACCGTTTCCAGCCCGTCCACATCTCTGCAGCGCTCAGATAGTTCCCAGCCTATG |
| | | TTGCTGAGAGTGGTTGGATCTCAGACCTCCGACTCAATGGGCGAAGAGGACTTGCTGTCTCCCCCA |
| | | CAGGACACTAGCACAGGACTCGAAGAGGTGATGGAACAGCTCAATAACTCTTTTCCTTCTAGTCGG |
| | | GGTCGCAATACCCCTGGAAAGCCCATGCGCGAAGACACCATGTAATAGCTCGAGAATCAACCTCTG |
| | | GATTACAAAATTTGTGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCTTTCCGGGACTTT |
| | | CGCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGGGAAGATGT |
| | | CTACTGAGCTGTGCGATCCCTGCTGGGGACTTTCCGCTGGGGACTTTCCGCTGGGGACTTTCCGCCT |
| | | TCAGCTAAGGAAGCTACCAATATTTAGAGGTACATTTTGTTCTAGAACAAAATGTACCGGTACATTTT |
| | | GTTCTGGTACATTTTGTTCT |
| 22. | CBA-hBGi- | CGTTACATAACTTACGGTAAATGGCCCGCCTGGCTGACCGCCCAACGACCCCCGCCCATTGACGTC |
| | DMD-3- | AATAATGACGTATGTTCCCATAGTAACGCCAATAGGGACTTTCCATTGACGTCAATGGGTGGAGTATT |
| | WPRE3- | TACGGTAAACTGCCCACTTGGCAGTACATCAAGTGTATCATATGCCAAGTACGCCCCCTATTGACGT |
| | DTS | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | CATGCTGTGGTGGGAAGAGGTGGAGGACTGTTACGAGAGAGAGGACGTGCAGAAGAAGACATTC |
| | | ACCAAGTGGGTGAACGCTCAGTTCAGCAAGTTCGGCAAGCAGCACATCGAGAACCTGTTTAGCGA |
| | | TCTACAAGATGGCAGAAGACTGCTGGACCTGCTGGAGGGCCTGACCGGGCAGAAGCTGCCCAAAG |
| | | AAAAGGGGTCTACAAGAGTGCACGCCCTGAACAACGTGAACAAGGCCCTGAGAGTGCTGCAGAA |
| | | CAACAACGTGGACCTGGTGAACATCGGCAGCACCGACATCGTGGACGGCAACCACAAGCTGACCC |
| | | TGGGCCTGATCTGGAACATCATCCTGCACTGGCAAGTGAAGAACGTGATGAAGAACATCATGGCCG |
| | | GCCTGCAGCAGACCAACAGCGAGAAGATCCTGCTGAGCTGGGTGAGACAGAGCACAAGAAACTA |
| | | CCCCCAAGTGAACGTGATCAACTTCACCACAAGCTGGAGCGACGGCCTGGCCCTGAACGCCCTGA |
| | | TCCACAGCCACAGACCCGACCTGTTCGACTGGAACAGCGTGGTGTGTCAGCAGAGCGCCACACAG |
| | | AGACTGGAGCACGCCTTCAACATCGCTAGATATCAGCTGGGCATCGAGAAGTTACTGGACCCCGAA |
| | | GATGTGGACACCACCTACCCCGACAAGAAGAGCATCCTGATGTACATCACAAGCCTGTTCCAAGTG |
| | | CTGCCTCAGCAAGTGAGCATCGAGGCCATCCAAGAAGTCGAGATGCTGCCTAGACCCCCCAAGGT |
| | | GACCAAGGAGGAGCACTTTCAGCTGCACCATCAGATGCACTACTCTCAGCAGATCACAGTCAGCTT |
| | | GGCACAAGGCTACGAGAGAACAAGCAGCCCCAAGCCTAGATTCAAGAGCTACGCCTACACCCAAG |
| | | CCGCCTACGTGACCACAAGTGACCCCACAAGAAGCCCCTTCCCTTCTCAGCACCTGGAGGCCCCCG |
| | | AGGACAAGAGCTTCGGCAGCAGCCTGATGGAGAGCGAGGTGAACCTGGACAGATATCAGACCGCG |
| | | TTAGAAGAAGTGTTGAGCTGGCTGCTGAGCGCCGAGGACACCCTGCAAGCCCAAGGCGAGATCAG |
| | | CAACGATGTGGAGGTGGTGAAGGATCAGTTCCACACCCACGAGGGCTACATGATGGACCTGACCG |
| | | CCCACCAAGGCAGAGTGGGCAACATCCTGCAGCTGGGCAGCAAGCTGATCGGCACCGGCAAGCTG |
| | | AGCGAGGACGAGGAAACCGAGGTTCAAGAGCAAATGAACCTGCTAAACAGCAGATGGGAGTGCC |
| | | TGAGAGTGGCTAGCATGGAAAAGCAGAGTAACCTGCACAGAGTGCTGATGGACCTGCAGAATCAG |
| | | AAATTGAAAGAACTTAATGATTGGCTGACCAAGACCGAGGAGAGAACAAGAAAGATGGAGGAGG |
| | | AGCCCCTGGGCCCCGACCTGGAGGACCTGAAGAGACAAGTGCAGCAGCACAAGGTGCTGCAAGA |
| | | GGACCTGGAGCAAGAGCAAGTGAGAGTGAACTCTCTGACACACATGGTGGTGGTAGTGGACGAGA |
| | | GCAGCGGCGATCACGCTACCGCGGCGCTGGAAGAGCAGCTGAAAGTGCTGGGCGACAGATGGGCC |
| | | AACATCTGCAGATGGACCGAGGACAGATGGGTGCTGCTGCAAGACATCCTGCTGAAGTGGCAGAG |
| | | ACTGACCGAGGAGCAGTGCCTGTTCAGCGCCTGGCTGAGCGAGAAGGAAGACGCTGTGAACAAG |
| | | ATCCACACCACCGGCTTCAAGGATCAGAACGAGATGCTGAGCTCCCTCCAAAAGCTCGCAGTGCTG |
| | | AAGGCCGACCTGGAAAAGAAAAAACAGTCGATGGGCAAGCTGTACAGCCTGAAGCAAGACCTGC |
| | | TGAGCACCCTGAAGAACAAGAGCGTGACACAGAAGACCGAGGCCTGGCTGGACAACTTCGCTAG |
| | | ATGCTGGGACAACCTGGTACAGAAGTTAGAGAAGTCTACCGCTCAGATCAGCCAAGCCGTAACCAC |
| | | CACACAACCTAGCCTGACTCAGACCACCGTGATGGAAACCGTGACCACCGTGACAACCCGTGAGC |
| | | AGATCCTGGTGAAGCACGCCCAAGAGGAGCTGCCCCCCCCCCCCCCTCAGAAGAAGAGACAGATC |
| | | ACCGTGGACTCAGAGATTCGCAAGAGACTGGACGTGGACATCACCGAGCTGCACAGCTGGATCAC |
| | | AAGAAGCGAGGCCGTGCTGCAGAGCCCTGAGTTCGCGATCTTTAGAAAGGAGGGCAACTTCAGCG |
| | | ACTTGAAGGAGAAAGTGAACGCCATCGAGAGAGAGAAGGCCGAGAAGTTCAGAAAGTTGCAAGA |
| | | CGCCTCAAGAAGCGCCCAAGCCCTGGTGGAGCAGATGGTTAACGAAGGCGTGAATGCCGACAGCA |
| | | TCAAGCAAGCTAGCGAGCAGCTGAATAGCCGGTGGATTGAATTCTGTCAGCTGCTGAGCGAGAGAT |
| | | TAAACTGGCTGGAGTATCAGAACAACATCATCGCCTTCTACAATCAGCTGCAGCAACTAGAACAAA |
| | | TGACCACCACCGCCGAGAACTGGCTAAAGATTCAGCCCACCACCCCTAGCGAGCCCACCGCCATCA |
| | | AATCTCAGCTGAAAATTTGCAAGGACGAAGTGAACAGACTGTCGGACCTGCAGCCTCAAATCGAA |
| | | AGACTGAAAATTCAGAGCATCGCACTTAAGGAGAAGGGCCAAGGACCCATGTTCCTGGACGCCGA |
| | | CTTCGTGGCCTTCACCAACCACTTCAAGCAAGTGTTCAGCGACGTGCAAGCTAGAGAGAAGGAGC |
| | | TGCAGACCATCTTCGACACCCTGCCCCCCATGAGATACCAAGAAACCATGAGCGCCATCAGAACCT |
| | | GGGTGCAGCAGAGCGAAACCAAGCTGAGCATCCCTCAGCTGAGCGTGACCGACTACGAGATCATG |
| | | GAGCAAAGACTGGGAGAGCTGCAAGCCCTGCAGAGCAGCCTGCAAGAGCAGCAGAGCGGCCTGT |
| | | ACTACCTGAGCACCACCGTGAAGGAGATGTCGAAGAAAGCCCCGTCGGAGATCAGCAGAAAGTAT |
| | | CAGAGCGAGTTCGAGGAGATCGAGGGCAGATGGAAGAAGCTGAGCAGCCAACTGGTGGAACACT |
| | | GTCAGAAATTAGAAGAACAGATGAACAAGCTGAGAAAGATTCAGAACCACATTCAGACCCTGAAG |
| | | AAGTGGATGGCCGAGGTGGACGTGTTCCTGAAAGAGGAGTGGCCCGCCCTGGGCGATTCCGAAAT |
| | | CTTAAAAAAGCAGCTGAAGCAGTGCAGACTGCTGGTGTCAGATATCCAAACCATTCAACCAAGCCT |
| | | GAACAGCGTGAACGAAGGCGGGCAGAAGATCAAGAACGAGGCCGAGCCCGAGTTCGCTAGCAGA |
| | | CTGGAAACCGAGCTGAAGGAGCTGAATACACAGTGGGACCACATGTGTCAGCAAGTGTACGCTAG |
| | | AAAGGAAGCGCTGAAGGGCGGGCTGGAGAAGACCGTTAGCCTGCAAAAGGATCTGAGCGAGATG |
| | | CACGAGTGGATGACCCAAGCCGAGGAGGAGTACCTGGAGAGAGACTTCGAGTACAAGACCCCCG |
| | | ACGAGCTGCAGAAGGCCGTCGAGGAAATGAAGAGAGCGAAGGAGGAGGCTCAGCAGAAGGAAG |
| | | CCAAGGTGAAGCTGCTGACTGAATCTGTCAACAGTGTGATAGCTCAAGCACCCCCCGTTGCCCAAG |
| | | AGGCACTAAAGAAGGAGCTGGAAACTCTGACCACCAACTATCAGTGGCTGTGCACAAGACTGAAC |
| | | GGCAAGTGCAAGACCCTGGAGGAAGTGTGGGCCTGCTGGCACGAGCTGCTGAGCTACCTGGAGAA |
| | | GGCCAACAAGTGGCTGAACGAGGTGGAGTTCAAGCTGAAGACCACCGAGAACATCCCCGGCGGC |
| | | GCCGAGGAGATCAGCGAGGTGCTGGACAGCCTGGAGAACCTGATGAGACACAGCGAGGACAACC |
| | | CCAATCAGATCAGAATCCTGGCTCAGACCCTGACCGACGGCGGCGTGATGGACGAGCTGATCAACG |
| | | AGGAGCTGGAAACCTTTAACTCTAGATGGCGAGAACTGCACGAGGAGGCCGTGAGAAGACAGAA |
| | | GCTGCTGGAGCAAAGCATTCAGAGCGCCCAAGAAACCGAGAAGAGCCTGCACCTGATCCAAGAG |
| | | AGCCTGACCTTCATCGACAAGCAGCTGGCCGCCTACATCGCCGACAAGGTGGACGCCGCTCAGATG |
| | | CCACAAGAGGCTCAGAAGATTCAGTCCGATCTGACAAGCCACGAGATCAGCCTGGAGGAGATGAA |
| | | AAAACACAACCAAGGCAAGGAGGCCGCTCAGAGAGTGCTGTCTCAGATCGACGTGGCTCAGAAG |
| | | AAGCTGCAAGACGTGAGCATGAAGTTCAGACTGTTTCAGAAGCCCGCCAACTTCGAGCAGCGACT |
| | | GCAAGAGAGCAAGATGATCCTGGACGAGGTGAAGATGCACCTGCCCGCCCTGGAAACCAAGAGCG |
| | | TGGAGCAAGAGGTGGTGCAGTCTCAGTTGAACCACTGCGTGAACCTGTACAAGAGCCTGTCGGAG |
| | | GTCAAGAGTGAGGTCGAGATGGTGATCAAAACTGGGAGACAGATCGTGCAAAAAAAGCAGACCG |
| | | AGAACCCCAAGGAGCTGGACGAGCGTGTTACCGCCCTGAAGCTGCACTACAACGAGCTGGGCGCC |
| | | AAGGTGACCGAGAGAAAGCAGCAGCTGGAGAAGTGCCTGAAGCTGAGCAGAAAGATGAGAAAG |
| | | GAGATGAACGTGCTGACCGAGTGGCTGGCCGCCACCGACATGGAGCTGACCAAGAGAAGCGCCGT |
| | | GGAGGGCATGCCTAGCAACCTGGACAGCGAGGTGGCCTGGGGCAAGGCCACACAGAAGGAGATC |
| | | GAGAAGCAGAAGGTGCACCTGAAGAGCATCACCGAGGTGGGCGAGGCCCTGAAGACCGTGCTGG |
| | | GCAAGAAGGAAACCCTGGTGGAGGACAAGCTGAGCCTGCTGAACTCGAACTGGATCGCCGTGAC |
| | | AAGCAGAGCCGAGGAGTGGCTGAACCTGCTGTTAGAGTACCAAAAACACATGGAAACCTTCGATC |
| | | AGAACGTGGACCACATCACCAAGTGGATCATCCAAGCCGACACCCTGCTGGACGAAAGCGAAAAG |
| | | AAGAAGCCGCAACAAAAGGAGGATGTGCTGAAGAGACTGAAGGCCGAACTGAACGATATCAGAC |
| | | CCAAGGTGGACAGCACACGGGACCAAGCCGCCAACCTGATGGCCAACAGAGGCGACCACTGCAG |
| | | AAAACTGGTGGAGCCTCAGATCAGCGAGCTGAACCACAGATTCGCCGCCATCAGCCACAGAATCA |
| | | AAACCGGCAAAGCGAGCATCCCCTTGAAGGAACTGGAGCAATTTAACAGCGACATCCAAAAATTG |
| | | CTCGAACCACTGGAGGCCGAGATTCAGCAAGGCGTGAACCTGAAGGAAGAGGACTTCAACAAGG |
| | | ACATGAACGAGGACAACGAGGGCACCGTGAAAGAACTGCTGCAGAGAGGCGACAACCTGCAGCA |
| | | GAGAATCACCGACGAGAGAAAGAGAGAGGAGATCAAGATCAAGCAGCAGCTGCTGCAGACTAAG |
| | | CACAACGCCCTGAAGGATCTGAGATCTCAGAGGAGAAAAAAGGCTCTGGAAATTTCACATCAGTG |
| | | GTATCAGTACAAGAGACAAGCAGACGACCTGCTGAAGTGCCTGGACGACATCGAGAAGAAGTTAG |
| | | CTAGCCTGCCCGAGCCTAGAGACGAGAGAAAGATCAAGGAGATCGACAGAGAGTTACAAAAGAA |
| | | GAAGGAGGAGCTGAATGCGGTAAGAAGACAAGCGGAAGGCCTGTCCGAGGACGGCGCAGCCATG |
| | | GCCGTGGAGCCCACACAGATTCAGCTCAGCAAGAGATGGAGAGAGATCGAGAGCAAGTTCGCTCA |
| | | GTTCAGAAGACTGAACTTCGCTCAGATCCACACCGTGAGAGAGGAAACCATGATGGTGATGACCG |
| | | AGGACATGCCCCTGGAGATCTCATACGTTCCTAGCACCTACCTGACCGAGATCACCCACGTGAGCC |
| | | AAGCCCTGCTGGAGGTGGAGCAGCTGCTGAACGCCCCCGACCTGTGCGCCAAGGACTTCGAGGAC |
| | | CTGTTCAAACAAGAGGAGAGCCTGAAGAACATCAAGGACAGCCTGCAGCAAAGTAGCGGCAGAA |
| | | TCGACATCATCCACAGCAAGAAGACCGCCGCCCTGCAGAGCGCCACCCCCGTGGAGAGAGTGAAG |
| | | CTTCAAGAGGCCCTTTCGCAGCTGGATTTTCAGTGGGAGAAGGTGAACAAAATGTACAAGGACAG |
| | | ACAAGGCAGATTCGACAGAAGCGTGGAGAAGTGGAGAAGATTCCACTACGACATCAAGATCTTCA |
| | | ATCAGTGGCTGACCGAGGCAGAGCAGTTCCTGAGAAAGACACAGATCCCCGAGAACTGGGAGCAC |
| | | GCCAAGTACAAGTGGTACCTGAAGGAGCTGCAAGACGGCATCGGGCAGAGACAGACCGTGGTGA |
| | | GAACCCTGAACGCCACCGGCGAGGAGATCATCCAACAAAGCTCCAAAACCGACGCTAGCATCCTG |
| | | CAAGAGAAACTGGGCAGCCTGAACCTGAGATGGCAAGAGGTGTGCAAGCAGCTGAGCGACCGCA |
| | | AAAAACGGTTAGAGGAGCAGAAGAACATCCTGAGCGAGTTTCAGCGGGACCTGAACGAGTTCGTG |
| | | CTGTGGCTGGAGGAGGCCGATAACATCGCTAGCATCCCCCTGGAGCCCGGCAAGGAGCAGCAGTTA |
| | | AAAGAAAAACTTGAGCAAGTGAAGCTGCTGGTGGAGGAGCTGCCCCTGAGACAAGGCATCCTGA |
| | | AGCAGCTGAACGAGACTGGCGGCCCCGTGCTGGTGAGCGCCCCCATCAGCCCCGAGGAGCAAGAC |
| | | AAGCTGGAGAACAAGCTGAAGCAGACCAACCTGCAGTGGATCAAGGTGAGCAGAGCCCTGCCCG |
| | | AGAAGCAAGGCGAAATCGAGGCCCAAATAAAGGACCTGGGGCAGCTGGAGAAGAAACTCGAAGA |
| | | CTTGGAAGAACAACTCAACCACTTGCTGCTGTGGCTGAGCCCCATCAGAAATCAGCTGGAGATCTA |
| | | CAATCAGCCCAACCAAGAGGGGCCATTCGACGTGAAGGAAACCGAGATCGCCGTGCAAGCCAAGC |
| | | AGCCCGATGTGGAGGAGATCCTGAGCAAGGGGCAGCACCTGTACAAGGAGAAGCCCGCCACACA |
| | | GCCCGTCAAGCGTAAACTAGAGGATCTTTCCTCTGAGTGGAAGGCCGTGAACCGTCTGCTACAAGA |
| | | GCTGCGGGCTAAGCAACCCGACTTAGCGCCCGGCCTGACCACCATCGGTGCAAGCCCCACGCAGA |
| | | CCGTGACCCTGGTGACCCAACCCGTGGTGACCAAGGAAACCGCCATCAGCAAGCTGGAGATGCCT |
| | | AGCAGCCTGATGCTGGAGGTGCCCGCCCTGGCCGACTTCAACAGAGCCTGGACCGAGCTGACCGA |
| | | CTGGCTGAGCCTGCTGGACCAAGTGATCAAGTCTCAGAGAGTGATGGTGGGCGATCTGGAGGACA |
| | | TCAATGAGATGATCATCAAGCAGAAGGCCACCATGCAAGACCTGGAGCAGAGAAGACCTCAGCTG |
| | | GAGGAGCTGATCACCGCCGCTCAGAACCTGAAAAACAAGACGAGCAACCAAGAGGCTAGAACCA |
| | | TCATCACCGACAGAATCGAGAGAATTCAGAATCAGTGGGACGAGGTGCAAGAGCACCTGCAGAAC |
| | | AGAAGACAGCAACTAAACGAAATGCTCAAGGACAGCACACAGTGGCTGGAGGCCAAGGAGGAGG |
| | | CGGAGCAAGTGTTGGGCCAAGCTAGAGCCAAGCTGGAGAGCTGGAAGGAGGGACCCTACACCGT |
| | | GGACGCCATTCAGAAGAAGATCACCGAAACCAAGCAGCTGGCCAAGGACCTGCGACAGTGGCAG |
| | | ACCAACGTGGACGTGGCCAACGATCTTGCCCTGAAGCTGCTGCGCGACTATAGCGCCGACGACAC |
| | | AAGAAAGGTGCACATGATCACCGAGAACATCAACGCTAGCTGGCGTAGCATCCACAAGAGAGTGA |
| | | GCGAGAGAGAGGCGGCTTTGGAGGAAACCCATAGACTGCTGCAGCAGTTTCCCCTGGACCTGGAG |
| | | AAGTTCCTGGCCTGGCTGACCGAAGCCGAAACAACCGCCAACGTGCTGCAAGACGCCACAAGAA |
| | | AGGAGAGACTGCTGGAGGACAGCAAGGGCGTGAAGGAGCTGATGAAGCAGTGGCAAGACCTGCA |
| | | AGGCGAGATCGAAGCGCACACCGACGTGTACCACAACCTGGACGAGAACTCTCAGAAGATCCTGA |
| | | GAAGCCTGGAGGGCAGCGACGACGCCGTGCTGTTACAGAGAAGGCTGGACAACATGAACTTCAAG |
| | | TGGAGCGAGCTGAGAAAGAAGAGCCTGAACATCAGAAGCCACCTGGAGGCTAGCAGCGATCAGT |
| | | GGAAGAGACTGCACCTGAGCCTGCAAGAGCTGCTGGTCTGGCTTCAGTTGAAGGACGATGAGCTG |
| | | AGCAGACAAGCCCCCATCGGCGGCGACTTCCCCGCCGTGCAGAAGCAGAACGACGTGCATAGAGC |
| | | CTTCAAGAGAGAGCTGAAGACCAAGGAGCCCGTGATCATGAGCACCCTGGAAACGGTGAGAATCT |
| | | TCCTGACCGAGCAGCCCCTGGAGGGCCTGGAGAAGCTGTACCAAGAGCCTAGAGAGCTGCCCCCC |
| | | GAGGAGAGAGCTCAGAACGTGACAAGACTGCTGAGAAAGCAAGCCGAAGAGGTGAACACCGAGT |
| | | GGGAGAAGCTGAACCTGCACAGCGCCGACTGGCAGAGAAAGATCGACGAAACCCTGGAGAGACT |
| | | GAGAGAATTGCAAGAGGCGACCGACGAGCTGGACCTGAAGCTGAGACAAGCCGAGGTCATCAAG |
| | | GGCAGCTGGCAGCCCGTGGGCGATCTATTAATTGACTCTCTACAAGACCACCTGGAGAAAGTGAAG |
| | | GCCCTGAGAGGCGAGATCGCCCCCCTGAAGGAGAACGTGAGCCACGTGAATGATCTGGCACGTCA |
| | | GCTGACCACCCTGGGCATACAACTGTCCCCCTATAACCTGAGCACACTGGAGGATTTGAATACTAGA |
| | | TGGAAGCTGCTGCAAGTGGCCGTGGAGGACAGAGTAAGACAATTACATGAGGCCCACAGAGACTT |
| | | CGGCCCCGCTTCTCAGCACTTCCTGAGCACAAGCGTGCAAGGCCCCTGGGAGAGAGCCATTTCCCC |
| | | CAACAAGGTCCCCTACTACATCAACCACGAGACACAGACCACCTGCTGGGACCACCCCAAGATGA |
| | | CCGAGCTGTATCAGAGCCTCGCCGATCTGAACAATGTGAGATTCAGCGCCTACAGAACCGCCATGA |
| | | AGCTGAGAAGACTGCAGAAGGCCCTGTGCCTGGACTTATTAAGCCTGAGCGCCGCCTGCGACGCC |
| | | CTGGATCAGCACAACCTGAAGCAAAACGACCAACCCATGGACATCCTGCAGATCATCAACTGCCTC |
| | | ACCACCATATACGACAGACTGGAACAAGAGCACAACAACCTGGTGAACGTGCCCCTGTGCGTGGA |
| | | CATGTGCCTGAACTGGCTGCTGAACGTGTACGACACCGGCAGAACCGGCAGAATCAGAGTGCTGA |
| | | GCTTCAAGACCGGCATCATCAGCCTGTGCAAGGCCCACCTGGAGGACAAATACAGATACCTTTTCA |
| | | AACAAGTGGCTAGCAGCACCGGCTTCTGCGACCAAAGAAGACTGGGTCTGCTGCTGCACGACAGC |
| | | ATTCAGATCCCTAGACAGCTGGGCGAGGTGGCTAGCTTCGGCGGCAGCAACATCGAGCCTAGCGTG |
| | | AGAAGCTGCTTTCAGTTCGCCAACAACAAGCCCGAGATAGAGGCAGCCCTCTTCTTAGACTGGATG |
| | | AGACTGGAGCCTCAGAGCATGGTATGGCTGCCGGTCCTGCACAGAGTGGCCGCCGCCGAAACCGC |
| | | CAAGCACCAAGCCAAGTGCAACATCTGCAAGGAGTGCCCCATCATCGGCTTCAGATACAGAAGCCT |
| | | GAAGCACTTCAACTACGACATCTGTCAGAGCTGCTTCTTCAGCGGCAGAGTGGCCAAGGGCCACA |
| | | AGATGCACTACCCCATGGTGGAGTACTGCACCCCCACCACAAGCGGCGAGGACGTGAGAGACTTC |
| | | GCCAAGGTGTTGAAGAATAAGTTTAGAACCAAGAGATACTTCGCCAAGCACCCTAGAATGGGCTAC |
| | | CTGCCCGTGCAGACCGTGCTGGAGGGCGACAACATGGAAACCCCCGTGACCCTGATCAACTTCTG |
| | | GCCCGTGGACAGCGCCCCCGCTAGCAGCCCTCAGCTGAGCCACGACGACACCCACAGCAGAATCG |
| | | AGCACTACGCTAGCAGACTGGCCGAGATGGAGAACAGCAACGGCAGCTACCTGAACGACAGCATC |
| | | AGCCCGAATGAGAGCATCGACGACGAGCACCTGCTGATTCAGCACTACTGTCAGAGCCTGAACCA |
| | | AGACAGCCCCCTGTCTCAGCCTAGAAGCCCCGCTCAGATCCTGATCAGCCTGGAGAGCGAGGAGA |
| | | GAGGCGAGCTGGAGAGAATCCTAGCAGATCTGGAGGAAGAGAACAGAAACCTGCAAGCCGAGTAT |
| | | GACCGACTGAAACAGCAACATGAGCACAAAGGCCTGAGCCCACTGCCCTCCCCCCCCGAGATGAT |
| | | GCCCACAAGCCCTCAGAGCCCTAGAGACGCCGAGCTGATCGCTGAAGCGAAGTTACTTAGACAAC |
| | | ACAAGGGCAGACTGGAGGCTAGAATGCAGATCCTGGAGGACCACAACAAGCAGCTGGAGTCTCAG |
| | | CTGCACAGACTGAGGCAGCTGCTGGAACAGCCCCAAGCCGAGGCCAAAGTGAACGGCACCACCG |
| | | TGAGCAGTCCTAGCACAAGCCTGCAGAGAAGCGACAGCTCTCAGCCCATGCTGCTGAGAGTGGTG |
| | | GGCTCTCAGACAAGCGACAGCATGGGCGAGGAGGATCTGCTGAGTCCGCCCCAAGACACAAGCAC |
| | | CGGCCTGGAAGAGGTGATGGAGCAACTGAACAATAGCTTCCCTAGCAGCAGAGGCAGAAACACCC |
| | | CCGGCAAGCCCATGAGAGAGGACACCATGTGATGACTCGAGAATCAACCTCTGGATTACAAAATTT |
| | | GTGAAAGATTGACTGGTATTCTTAACTATGTTGCTCCTTTTACGCTATGTGGATACGCTGCTTTAATGC |
| | | CTTTGTATCATGCTATTGCTTCCCGTATGGCTTTCATTTTCTCCTCCTTGTATAAATCCTGGTTGCTGTC |
| | | TCTTTATGAGGAGTTGTGGCCCGTTGTCAGGCAACGTGGCGTGGTGTGCACTGTGTTTGCTGACGC |
| | | AACCCCCACTGGTTGGGGCATTGCCACCACCTGTCAGCTCCTTTCCGGGACTTTCGCTTTCCCCCTC |
| | | CCTATTGCCACGGCGGAACTCATCGCCGCCTGCCTTGCCCGCTGCTGGACAGGGGCTCGGCTGTTG |
| | | GGCACTGACAATTCCGTGGGGTGTGCCTTCTAGTTGCCAGCCATCTGTTGTTTGCCCCTCCCCCGTG |
| | | CCTTCCTTGACCCTGGAAGGTGCCACTCCCACTGTCCTTTCCTAATAAAATGAGGAAATTGCATCGC |
| | | ATTGTCTGAGTAGGTGTCATTCTATTCTGGGGGGTGGGGTGGGGCAGGACAGCAAGGGGGAGGATT |
| | | GGGAAGACAATAGCAGGCATGCTGGGGATGCGGTGGGCTCTATGGGAAGATGTCTACTGAGCTGTG |
| | | CGATCCCTGCTGGGGACTTTCCGCTGGGGACTTTCCGCTGGGGACTTTCCGCCTTCAGCTAAGGAA |
| | | GCTACCAATATTTAGAGGTACATTTTGTTCTAGAACAAAATGTACCGGTACATTTTGTTCTGGTACAT |
| | | TTTGTTCT |
| 23. | CBA-hBGi- | CGTTACATAACTTACGGTAAATGGCCCGCCTGGCTGACCGCCCAACGACCCCCGCCCATTGACGTC |
| | DMD-4- | AATAATGACGTATGTTCCCATAGTAACGCCAATAGGGACTTTCCATTGACGTCAATGGGTGGAGTATT |
| | WPRE3- | TACGGTAAACTGCCCACTTGGCAGTACATCAAGTGTATCATATGCCAAGTACGCCCCCTATTGACGT |
| | DTS | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | CATGCTTTGGTGGGAAGAAGTCGAGGACTGCTACGAGCGCGAGGACGTGCAGAAGAAAACCTTCA |
| | | CCAAATGGGTCAACGCCCAGTTCAGCAAGTTCGGCAAGCAGCACATCGAGAACCTGTTCAGCGAC |
| | | CTGCAGGATGGCAGAAGGCTGCTGGATCTGCTGGAAGGCCTGACAGGACAGAAGCTGCCCAAAGA |
| | | GAAGGGCAGCACAAGAGTGCACGCCCTGAACAACGTGAACAAGGCCCTGAGAGTGCTGCAGAAC |
| | | AACAACGTGGACCTGGTCAACATCGGCAGCACCGACATCGTGGACGGCAACCACAAACTGACCCT |
| | | GGGCCTGATCTGGAACATCATCCTGCACTGGCAAGTGAAGAACGTGATGAAGAACATCATGGCCGG |
| | | CCTGCAGCAGACCAACAGCGAGAAGATTCTGCTGAGCTGGGTCCGACAGAGCACCCGGAATTACC |
| | | CTCAAGTGAACGTGATCAACTTCACCACCTCTTGGAGCGACGGACTGGCCCTGAATGCCCTGATCC |
| | | ACAGCCACAGACCTGACCTGTTCGACTGGAACAGCGTCGTGTGTCAGCAGAGCGCCACACAGAGG |
| | | CTGGAACACGCCTTCAATATCGCCAGATACCAGCTGGGCATCGAGAAACTGCTGGACCCCGAGGAT |
| | | GTGGACACCACCTATCCTGACAAGAAATCCATCCTCATGTACATCACCAGCCTGTTCCAGGTGCTGC |
| | | CCCAGCAGGTTTCCATCGAGGCCATTCAAGAGGTCGAGATGCTGCCCAGACCTCCTAAAGTGACCA |
| | | AAGAGGAACACTTCCAGCTGCACCACCAGATGCACTACTCTCAGCAGATCACCGTGTCTCTGGCCC |
| | | AGGGCTACGAGAGAACAAGCAGCCCCAAGCCTCGGTTCAAGAGCTACGCCTATACACAGGCCGCC |
| | | TACGTGACCACCAGCGATCCTACAAGAAGCCCATTTCCTAGCCAGCATCTGGAAGCCCCTGAGGAC |
| | | AAGAGCTTTGGCAGCAGCCTGATGGAAAGCGAAGTGAACCTGGACCGCTACCAGACAGCCCTGGA |
| | | AGAGGTTCTGAGCTGGCTGCTGTCTGCCGAGGATACACTGCAGGCTCAGGGCGAGATCAGCAACG |
| | | ACGTGGAAGTGGTCAAGGACCAGTTTCACACCCACGAGGGCTACATGATGGACCTGACAGCCCAC |
| | | CAGGGCAGAGTGGGCAATATTCTGCAGCTGGGCTCCAAGCTGATCGGCACAGGCAAGCTGAGCGA |
| | | GGACGAAGAGACAGAGGTGCAAGAGCAGATGAACCTGCTGAACAGCAGATGGGAGTGTCTGAGA |
| | | GTGGCCAGCATGGAAAAGCAGAGCAACCTGCACCGGGTGCTGATGGATCTCCAGAACCAGAAGCT |
| | | GAAAGAGCTGAACGACTGGCTGACCAAGACCGAGGAACGGACCCGGAAGATGGAAGAGGAACCT |
| | | CTGGGACCCGACCTGGAAGATCTGAAAAGACAGGTGCAGCAGCATAAGGTGCTGCAAGAGGACCT |
| | | CGAACAAGAGCAAGTGCGCGTGAACAGCCTGACACACATGGTGGTGGTCGTGGATGAGAGCAGC |
| | | GGAGATCATGCCACAGCCGCTCTGGAAGAACAGCTGAAGGTGCTGGGAGACAGATGGGCCAATAT |
| | | CTGCCGGTGGACCGAGGATAGATGGGTGCTGCTCCAGGACATCCTGCTGAAGTGGCAGCGGCTGA |
| | | CAGAGGAACAGTGCCTGTTTAGCGCCTGGCTGTCCGAGAAAGAGGACGCCGTCAACAAGATCCAC |
| | | ACCACCGGCTTCAAGGATCAGAATGAGATGCTGAGCAGCCTGCAGAAACTGGCCGTGCTGAAGGC |
| | | TGACCTGGAAAAGAAAAAGCAGTCCATGGGCAAGCTGTACTCCCTGAAGCAGGACCTGCTGAGCA |
| | | CACTGAAGAACAAGTCTGTGACCCAGAAAACCGAGGCCTGGCTGGACAACTTCGCCCGGTGTTGG |
| | | GATAACCTGGTGCAGAAGCTGGAAAAGTCCACCGCTCAGATCTCTCAGGCCGTGACCACAACACA |
| | | GCCTTCTCTGACCCAGACCACCGTGATGGAAACAGTGACCACAGTGACAACCCGCGAGCAGATCC |
| | | TGGTCAAGCACGCCCAAGAAGAACTGCCTCCTCCACCTCCTCAGAAGAAACGGCAGATCACAGTG |
| | | GACAGCGAGATCCGGAAGAGACTGGACGTGGACATCACCGAGCTGCACAGCTGGATCACCAGATC |
| | | CGAAGCCGTGCTGCAGTCCCCTGAGTTCGCCATCTTCAGAAAAGAGGGCAACTTCTCCGACCTGA |
| | | AAGAGAAAGTCAACGCCATCGAGAGAGAGAAGGCCGAGAAGTTCCGGAAGCTGCAGGACGCCTC |
| | | TAGATCTGCCCAGGCTCTGGTGGAACAGATGGTCAACGAAGGCGTGAACGCCGACAGCATCAAGC |
| | | AGGCCTCAGAGCAGCTGAACTCCCGGTGGATCGAGTTCTGTCAGCTCCTGAGCGAGAGACTGAAC |
| | | TGGCTGGAATACCAGAACAATATCATTGCCTTCTACAACCAGCTCCAGCAGCTCGAACAGATGACC |
| | | ACCACAGCCGAGAATTGGCTGAAGATCCAGCCTACCACACCTAGCGAGCCCACCGCCATTAAGAGC |
| | | CAGCTGAAAATCTGCAAGGACGAAGTGAATCGGCTGAGCGATCTGCAGCCCCAGATCGAAAGACT |
| | | GAAAATCCAGTCTATCGCCCTCAAAGAGAAAGGACAGGGCCCCATGTTCCTGGACGCCGATTTTGT |
| | | GGCCTTTACCAACCACTTCAAACAGGTGTTCTCCGACGTGCAGGCCCGGGAAAAAGAGCTGCAGA |
| | | CCATCTTCGACACCCTGCCTCCAATGAGATACCAAGAGACAATGAGCGCCATCCGGACCTGGGTGC |
| | | AGCAAAGCGAGACAAAGCTGAGCATCCCACAGCTGAGCGTGACCGACTACGAGATCATGGAACAG |
| | | AGACTGGGCGAACTGCAGGCCCTCCAGTCTAGCCTGCAAGAACAGCAGTCCGGCCTGTACTACCT |
| | | GAGCACAACCGTGAAAGAGATGAGCAAGAAGGCCCCTAGCGAGATCTCCCGGAAGTACCAGAGC |
| | | GAGTTCGAAGAGATCGAAGGCCGGTGGAAGAAGCTGTCTAGCCAGCTGGTTGAGCACTGCCAGAA |
| | | ACTCGAAGAACAAATGAACAAGCTGCGCAAGATCCAGAATCACATCCAGACGCTGAAGAAATGGA |
| | | TGGCCGAGGTGGACGTGTTCCTGAAAGAAGAGTGGCCCGCTCTGGGCGACTCCGAGATTCTGAAG |
| | | AAACAGCTCAAACAGTGCCGGCTGCTGGTGTCCGATATCCAGACAATCCAGCCAAGCCTGAACTCC |
| | | GTGAATGAAGGCGGCCAGAAGATCAAGAACGAGGCCGAGCCTGAGTTTGCCAGCAGACTGGAAA |
| | | CCGAACTGAAAGAACTCAACACCCAGTGGGACCACATGTGCCAGCAAGTGTACGCCCGGAAAGAG |
| | | GCCCTGAAAGGCGGACTCGAAAAGACTGTGTCTCTGCAGAAAGACCTGTCTGAGATGCACGAGTG |
| | | GATGACCCAGGCCGAAGAGGAATACCTGGAACGGGACTTCGAGTACAAGACCCCTGATGAGCTGC |
| | | AAAAAGCCGTCGAGGAAATGAAGCGGGCCAAAGAAGAGGCCCAGCAGAAAGAAGCCAAAGTCA |
| | | AGCTGCTGACCGAGTCCGTGAATAGCGTTATCGCTCAGGCCCCTCCTGTGGCTCAAGAGGCTCTCA |
| | | AGAAAGAACTGGAAACTCTGACCACCAACTACCAGTGGCTGTGCACCAGACTGAACGGCAAGTGC |
| | | AAGACACTGGAAGAAGTGTGGGCCTGCTGGCACGAACTGCTGTCCTATCTGGAAAAGGCCAACAA |
| | | GTGGCTGAACGAGGTGGAATTCAAGCTGAAAACCACCGAGAACATCCCTGGCGGCGCTGAAGAGA |
| | | TTAGCGAGGTGCTGGACAGCCTGGAAAACCTGATGAGACACAGCGAGGACAACCCCAATCAGATC |
| | | AGAATCCTGGCTCAGACCCTGACCGATGGCGGCGTGATGGACGAGCTGATCAACGAGGAACTCGA |
| | | AACCTTCAACAGCCGGTGGCGGGAACTGCACGAGGAAGCTGTTCGGAGGCAGAAGTTGCTGGAA |
| | | CAGTCTATCCAGAGCGCACAAGAAACCGAGAAGTCCCTGCACCTGATCCAAGAGAGCCTGACCTT |
| | | CATCGACAAGCAGCTGGCCGCTTATATCGCCGACAAGGTGGACGCTGCTCAGATGCCCCAAGAGGC |
| | | ACAGAAGATTCAGAGCGACCTGACCAGCCACGAGATTAGCCTGGAAGAAATGAAGAAGCACAACC |
| | | AGGGCAAAGAGGCCGCTCAGAGAGTCCTGAGCCAGATCGATGTGGCACAGAAAAAGCTCCAGGAT |
| | | GTGTCCATGAAGTTTCGGCTGTTTCAGAAGCCCGCCAACTTCGAGCAGAGACTGCAAGAGTCCAA |
| | | GATGATCCTGGACGAAGTCAAAATGCATCTGCCCGCACTGGAAACAAAGTCCGTGGAACAAGAAG |
| | | TGGTGCAGTCCCAGCTGAACCACTGCGTGAACCTGTACAAGAGCCTGTCCGAAGTGAAGTCTGAG |
| | | GTGGAAATGGTCATCAAGACCGGCAGGCAGATCGTCCAGAAGAAGCAGACCGAGAATCCCAAAGA |
| | | ACTCGACGAGAGAGTGACCGCTCTGAAGCTGCACTACAATGAGCTGGGCGCCAAAGTGACCGAGC |
| | | GGAAGCAACAGCTTGAGAAGTGCCTGAAACTGTCTCGGAAGATGCGGAAAGAAATGAACGTGCTG |
| | | ACAGAGTGGCTGGCCGCCACCGATATGGAACTGACTAAGAGAAGCGCCGTGGAAGGCATGCCCAG |
| | | CAACCTGGATAGTGAAGTGGCCTGGGGCAAAGCCACTCAGAAAGAGATTGAGAAGCAGAAGGTCC |
| | | ACCTGAAGTCCATCACCGAAGTGGGCGAAGCCCTGAAAACCGTGCTGGGAAAGAAAGAGACACT |
| | | GGTCGAGGACAAGCTGTCCCTGCTGAATTCCAACTGGATCGCCGTGACCTCCAGAGCTGAGGAATG |
| | | GCTGAATCTGCTGCTTGAGTACCAGAAACACATGGAAACGTTCGACCAGAACGTCGACCACATCAC |
| | | AAAGTGGATCATCCAGGCTGATACCCTGCTGGACGAGTCCGAGAAGAAGAAACCCCAACAAAAAG |
| | | AAGATGTGCTGAAGCGGCTGAAAGCCGAGCTGAATGACATCCGGCCTAAGGTGGACAGCACCAGA |
| | | GATCAGGCAGCCAACCTGATGGCCAACAGAGGCGACCACTGTCGGAAGCTCGTGGAACCTCAGAT |
| | | CAGCGAGCTTAACCACAGATTTGCCGCCATCAGCCACCGGATCAAGACAGGCAAGGCCAGCATTCC |
| | | TCTCAAAGAGCTTGAGCAGTTCAACAGCGACATCCAAAAGCTGCTCGAACCCCTGGAAGCCGAGA |
| | | TCCAACAGGGCGTCAACCTCAAAGAAGAAGATTTCAACAAGGACATGAACGAGGACAATGAGGG |
| | | CACCGTCAAAGAACTGCTGCAGCGGGGCGATAATCTGCAGCAGCGGATTACCGATGAGCGCAAGC |
| | | GGGAAGAGATCAAGATTAAGCAGCAGCTGCTGCAGACCAAGCACAACGCCCTGAAGGACCTGCGG |
| | | AGCCAGAGAAGAAAGAAGGCCCTGGAAATCAGCCACCAGTGGTATCAGTACAAGCGGCAGGCCG |
| | | ACGACCTGCTGAAGTGCCTGGATGACATCGAGAAGAAGCTGGCCTCTCTGCCCGAGCCTAGGGAC |
| | | GAGAGAAAGATCAAAGAGATCGACCGCGAGCTGCAGAAAAAGAAAGAGGAACTGAACGCCGTCC |
| | | GCAGACAGGCCGAAGGACTTTCTGAAGATGGCGCCGCTATGGCCGTGGAACCCACACAGATTCAG |
| | | CTGAGCAAGCGGTGGCGGGAAATCGAGAGCAAGTTCGCCCAGTTCCGGCGGCTGAATTTCGCCCA |
| | | GATCCACACCGTGCGGGAAGAGACAATGATGGTCATGACAGAGGACATGCCCCTTGAGATCAGCTA |
| | | CGTGCCCAGCACCTACCTGACCGAGATCACCCATGTGTCTCAGGCCCTGCTGGAAGTGGAACAGCT |
| | | GCTGAATGCCCCTGACCTGTGCGCCAAGGACTTCGAGGACCTGTTCAAGCAAGAGGAAAGCCTGA |
| | | AGAACATCAAGGACAGCCTGCAGCAGAGCAGCGGCCGGATCGATATCATCCACAGCAAGAAAACC |
| | | GCCGCACTGCAGAGCGCCACACCTGTGGAAAGAGTGAAGCTGCAAGAGGCCCTGAGCCAGCTGG |
| | | ATTTCCAGTGGGAGAAAGTGAACAAGATGTACAAGGACCGGCAGGGCAGATTCGACCGCAGCGTT |
| | | GAAAAGTGGCGGCGGTTCCACTACGACATCAAGATCTTCAACCAGTGGCTGACCGAGGCCGAGCA |
| | | GTTCCTGAGAAAGACACAGATCCCCGAGAACTGGGAGCACGCCAAGTACAAGTGGTATCTGAAAG |
| | | AGCTGCAGGACGGCATCGGCCAGAGACAGACAGTCGTGCGGACACTGAATGCCACCGGCGAGGA |
| | | AATCATCCAGCAGTCCAGCAAGACCGACGCCAGCATCCTGCAAGAGAAGCTGGGCAGCCTGAACC |
| | | TGCGGTGGCAAGAAGTGTGCAAGCAGCTGAGCGACCGGAAGAAGCGGCTGGAAGAACAGAAGA |
| | | ATATCCTGAGCGAGTTCCAGCGGGACCTGAACGAGTTCGTGCTGTGGCTCGAAGAGGCCGACAATA |
| | | TCGCCAGCATTCCCCTGGAACCTGGCAAAGAGCAGCAGCTGAAAGAAAAGCTGGAACAAGTGAA |
| | | ACTGCTGGTCGAGGAACTGCCCCTGAGACAGGGAATCCTGAAACAGCTGAACGAGACAGGCGGC |
| | | CCTGTGCTGGTGTCTGCCCCTATTTCTCCCGAGGAACAGGACAAGCTGGAAAACAAGCTGAAGCA |
| | | GACCAACCTGCAGTGGATCAAGGTGTCCAGAGCACTGCCTGAGAAGCAGGGCGAGATCGAGGCCC |
| | | AGATCAAGGACCTGGGACAGCTCGAAAAGAAGCTTGAGGACCTCGAAGAACAGCTCAACCATCTG |
| | | CTGCTTTGGCTGAGCCCCATCCGGAACCAGCTGGAAATCTACAACCAGCCTAATCAAGAGGGCCCC |
| | | TTCGACGTGAAAGAAACCGAGATTGCCGTGCAGGCCAAGCAGCCCGATGTGGAAGAGATCCTGTC |
| | | TAAGGGCCAGCACCTGTACAAAGAGAAGCCCGCCACACAGCCCGTGAAGCGGAAACTGGAAGAT |
| | | CTGAGCAGCGAGTGGAAGGCCGTGAACCGGCTGCTGCAAGAACTGAGAGCCAAACAGCCTGATCT |
| | | GGCCCCTGGCCTGACAACAATTGGCGCCTCTCCTACACAGACCGTGACACTGGTCACACAGCCTGT |
| | | GGTCACCAAAGAGACAGCCATCAGCAAACTGGAAATGCCCAGCAGTCTGATGCTCGAAGTGCCCG |
| | | CACTGGCCGACTTCAATAGAGCCTGGACCGAGCTGACCGACTGGCTCAGTCTGCTGGACCAAGTC |
| | | ATCAAGTCCCAGAGAGTGATGGTCGGAGATCTTGAGGACATCAACGAGATGATCATTAAGCAGAAA |
| | | GCCACCATGCAGGACCTTGAGCAGAGAAGGCCCCAGCTTGAAGAACTGATCACCGCCGCTCAGAA |
| | | CCTGAAAAACAAGACCAGCAATCAAGAAGCCCGGACCATCATCACCGACCGGATCGAGAGAATCC |
| | | AGAACCAGTGGGACGAAGTGCAAGAGCATCTGCAGAACAGACGGCAGCAGCTCAATGAGATGCT |
| | | GAAGGACAGCACACAGTGGCTGGAAGCCAAAGAAGAAGCCGAGCAGGTTCTCGGACAGGCCAGA |
| | | GCTAAGCTGGAATCTTGGAAAGAGGGACCCTACACCGTCGACGCCATCCAGAAGAAGATCACCGA |
| | | GACAAAGCAGCTGGCCAAGGATCTGAGACAGTGGCAGACCAATGTGGACGTGGCCAACGACCTG |
| | | GCTCTGAAGCTGCTGAGAGACTACAGCGCCGACGACACCAGAAAGGTGCACATGATCACCGAAAA |
| | | CATCAACGCCTCTTGGCGGAGCATCCACAAGCGGGTGTCAGAGAGAGAAGCCGCTCTGGAAGAAA |
| | | CACATAGACTGCTCCAGCAGTTCCCACTCGACCTGGAAAAGTTCCTGGCCTGGCTGACAGAAGCCG |
| | | AAACCACCGCTAACGTGCTCCAGGATGCCACCAGAAAAGAGCGGCTGTTGGAGGACAGCAAGGG |
| | | CGTCAAAGAACTCATGAAGCAGTGGCAGGATCTGCAAGGGGAGATTGAAGCCCACACCGACGTGT |
| | | ACCACAACCTGGACGAGAACAGCCAGAAGATCCTGCGGTCCCTGGAAGGCTCTGATGATGCTGTG |
| | | CTGCTTCAGAGGCGGCTGGACAACATGAACTTCAAGTGGAGCGAGCTGCGGAAGAAGTCCCTGAA |
| | | CATCAGAAGCCACCTGGAAGCCTCCAGCGACCAGTGGAAAAGACTGCACCTGTCTCTCCAAGAGC |
| | | TGCTCGTGTGGCTGCAGCTGAAGGATGACGAGCTGAGTAGACAGGCCCCTATCGGCGGAGATTTTC |
| | | CCGCCGTGCAGAAACAGAACGACGTGCACCGGGCCTTCAAGAGAGAGCTGAAAACAAAAGAACC |
| | | CGTCATCATGAGCACCCTGGAAACCGTGCGGATCTTTCTGACCGAGCAGCCTCTGGAAGGACTGGA |
| | | AAAGCTGTACCAAGAGCCTAGAGAGCTGCCTCCTGAAGAAAGGGCCCAGAACGTGACACGGCTGC |
| | | TTAGAAAGCAGGCCGAGGAAGTGAACACCGAATGGGAGAAGCTGAACCTCCACTCCGCCGACTGG |
| | | CAGCGGAAGATCGATGAGACACTGGAACGGCTGCGGGAACTGCAAGAAGCCACAGACGAGCTGG |
| | | ACCTGAAACTGCGGCAGGCTGAAGTGATCAAAGGCTCCTGGCAGCCAGTGGGCGATCTGCTGATC |
| | | GATAGCCTGCAGGACCATCTGGAAAAAGTCAAGGCCCTGCGGGGAGAGATCGCCCCTCTCAAAGA |
| | | AAACGTGTCCCACGTGAACGATCTGGCCAGGCAGCTGACAACACTGGGCATCCAGCTGAGCCCTT |
| | | ACAACCTGTCTACCTTGGAGGACCTGAATACCCGGTGGAAGCTGCTCCAAGTGGCCGTCGAGGATA |
| | | GAGTGCGGCAGCTGCATGAGGCTCACAGAGATTTTGGACCCGCCAGCCAGCACTTCCTGAGCACAT |
| | | CTGTTCAAGGCCCCTGGGAGAGAGCTATCAGCCCTAACAAGGTGCCCTACTACATCAACCACGAGA |
| | | CACAGACCACCTGTTGGGATCACCCCAAGATGACCGAACTGTATCAGTCCCTGGCCGACCTGAACA |
| | | ACGTGCGGTTTAGCGCCTACCGGACCGCCATGAAGCTGCGCAGACTGCAGAAAGCTCTGTGCCTG |
| | | GACCTGCTGTCTCTGAGCGCTGCTTGTGATGCCCTGGACCAGCATAACCTCAAGCAGAACGACCAG |
| | | CCTATGGACATCCTGCAGATCATCAACTGCCTGACCACCATCTACGACCGGCTCGAACAAGAGCAC |
| | | AACAACCTGGTCAACGTGCCCCTGTGCGTGGACATGTGCCTGAATTGGCTGCTGAACGTGTACGAC |
| | | ACCGGCAGAACCGGCAGGATCAGAGTGCTGAGCTTCAAGACCGGCATCATCTCCCTGTGCAAGGC |
| | | CCACCTTGAGGATAAGTACCGCTACCTGTTTAAGCAGGTCGCCAGCAGCACCGGCTTTTGCGACCA |
| | | AAGAAGGCTGGGACTGCTGCTCCACGACAGCATTCAGATCCCTAGACAGCTGGGCGAAGTGGCCT |
| | | CTTTCGGCGGCTCTAATATCGAGCCTAGCGTGCGGAGCTGCTTCCAGTTCGCCAACAACAAGCCCG |
| | | AGATTGAGGCCGCACTGTTCCTGGACTGGATGAGACTGGAACCCCAGAGCATGGTCTGGCTGCCTG |
| | | TGCTGCATAGAGTGGCCGCAGCCGAAACAGCCAAGCACCAGGCCAAGTGCAACATCTGCAAAGAG |
| | | TGCCCCATCATCGGCTTCCGGTACAGATCCCTGAAGCACTTCAACTACGATATCTGCCAGTCCTGCT |
| | | TCTTCAGCGGCAGAGTGGCCAAGGGCCACAAGATGCACTACCCCATGGTGGAATACTGCACCCCTA |
| | | CCACCTCTGGCGAGGACGTGCGGGATTTTGCCAAGGTGCTCAAGAACAAGTTCCGGACCAAGCGC |
| | | TACTTCGCTAAGCACCCCAGAATGGGCTACCTGCCAGTGCAGACAGTGCTTGAGGGCGACAACATG |
| | | GAAACCCCTGTGACTCTGATCAACTTCTGGCCCGTGGATAGCGCCCCTGCTAGTTCTCCTCAGCTGT |
| | | CTCACGACGATACCCACAGCAGAATCGAGCACTACGCCTCCAGACTGGCCGAGATGGAAAACAGC |
| | | AACGGCAGCTACCTGAATGACAGCATCAGCCCCAACGAGAGCATCGACGACGAACATCTGCTCATT |
| | | CAGCACTACTGCCAGAGCCTGAATCAGGACAGCCCTCTGTCTCAGCCTAGAAGCCCTGCACAGATC |
| | | CTGATCAGCCTGGAAAGCGAGGAACGCGGCGAGCTGGAAAGAATCCTGGCAGACCTGGAAGAGG |
| | | AAAACCGGAACCTGCAGGCCGAATACGACAGACTCAAGCAGCAGCACGAGCACAAGGGACTGTC |
| | | TCCACTGCCTTCTCCACCTGAAATGATGCCCACCTCTCCACAGAGCCCCAGAGATGCCGAGCTGAT |
| | | TGCCGAAGCCAAACTGCTGAGGCAGCACAAAGGCAGACTCGAAGCCAGAATGCAGATTCTCGAAG |
| | | ATCACAACAAGCAGCTCGAATCCCAGCTGCACAGACTTAGGCAGCTGCTGGAACAGCCTCAGGCA |
| | | GAGGCCAAAGTGAATGGCACCACCGTGTCTAGCCCTAGCACAAGCCTGCAGAGATCCGACAGCAG |
| | | CCAGCCAATGCTTCTGAGAGTCGTGGGCTCCCAGACCAGCGATTCTATGGGCGAAGAGGACCTGCT |
| | | CAGCCCTCCTCAGGATACAAGCACCGGCCTGGAAGAAGTGATGGAACAACTGAACAACAGCTTCC |
| | | CCAGCAGCAGAGGCAGAAACACCCCTGGCAAGCCCATGCGCGAGGACACCATGTGATAACTCGAG |
| | | AATCAACCTCTGGATTACAAAATTTGTGAAAGATTGACTGGTATTCTTAACTATGTTGCTCCTTTTAC |
| | | GCTATGTGGATACGCTGCTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGGGGACTTTCCGCTG |
| | | GGGACTTTCCGCCTTCAGCTAAGGAAGCTACCAATATTTAGAGGTACATTTTGTTCTAGAACAAAAT |
| | | GTACCGGTACATTTTGTTCTGGTACATTTTGTTCT |
| 24. | CBA-hBGi- | CGTTACATAACTTACGGTAAATGGCCCGCCTGGCTGACCGCCCAACGACCCCCGCCCATTGACGTC |
| | DMD-5- | AATAATGACGTATGTTCCCATAGTAACGCCAATAGGGACTTTCCATTGACGTCAATGGGTGGAGTATT |
| | WPRE3- | TACGGTAAACTGCCCACTTGGCAGTACATCAAGTGTATCATATGCCAAGTACGCCCCCTATTGACGT |
| | DTS | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | CATGCTGTGGTGGGAGGAAGTTGAAGATTGTTATGAGAGAGAAGATGTCCAGAAAAAGACCTTTAC |
| | | CAAATGGGTGAACGCACAGTTCTCCAAATTTGGGAAACAGCATATAGAAAATTTGTTCAGCGATCTT |
| | | CAGGATGGGCGCAGGTTGCTTGACCTTCTGGAAGGACTGACCGGGCAGAAGCTGCCCAAAGAGAA |
| | | AGGGTCTACCAGAGTTCACGCTCTGAACAACGTGAATAAGGCACTGCGAGTCCTGCAAAATAACA |
| | | ACGTCGACCTGGTGAACATAGGCAGCACTGATATTGTTGATGGGAACCATAAACTCACACTGGGTC |
| | | TGATTTGGAATATTATACTGCACTGGCAGGTCAAAAATGTTATGAAAAATATTATGGCCGGGCTTCAA |
| | | CAGACCAATTCCGAGAAGATCCTGCTGTCTTGGGTTAGGCAGTCCACGAGGAACTACCCTCAAGTG |
| | | AACGTTATTAACTTCACAACATCATGGAGTGATGGACTTGCCCTGAACGCCTTGATCCACTCCCATA |
| | | GGCCAGACCTGTTCGATTGGAATAGCGTAGTCTGTCAGCAGAGTGCTACACAACGCCTGGAACACG |
| | | CCTTTAACATAGCCAGGTATCAGCTTGGAATTGAGAAACTCCTCGACCCTGAAGACGTAGACACCA |
| | | CTTACCCCGACAAAAAATCCATTCTTATGTACATAACTTCCTTGTTTCAGGTTCTCCCCCAGCAGGTG |
| | | TCCATCGAGGCTATACAGGAGGTCGAAATGCTCCCCAGACCCCCTAAGGTCACGAAGGAAGAGCA |
| | | CTTCCAACTGCACCATCAGATGCACTACAGTCAACAGATCACTGTGAGTCTCGCACAGGGGTACGA |
| | | AAGGACCAGCTCCCCTAAACCCCGCTTCAAGTCCTACGCCTATACTCAGGCAGCTTACGTAACCAC |
| | | AAGTGACCCCACGCGGTCACCCTTCCCAAGCCAGCACCTGGAGGCTCCCGAGGACAAAAGCTTTG |
| | | GATCATCCCTGATGGAATCTGAAGTCAATCTCGATAGGTACCAAACCGCTCTGGAGGAGGTCCTCA |
| | | GTTGGCTCCTTTCAGCTGAAGATACTCTGCAGGCACAGGGTGAGATCAGCAATGACGTGGAGGTAG |
| | | TGAAAGATCAGTTTCACACTCACGAGGGCTATATGATGGACCTGACAGCCCATCAAGGTCGCGTTG |
| | | GCAATATCCTGCAGTTGGGGTCCAAACTGATTGGTACCGGTAAGTTGTCCGAGGACGAAGAGACAG |
| | | AAGTGCAGGAGCAGATGAACCTGCTTAATTCTAGATGGGAATGCCTCCGCGTGGCCTCTATGGAGA |
| | | AGCAGTCCAATCTCCATCGGGTTCTCATGGACTTGCAGAATCAGAAGCTGAAGGAACTCAACGACT |
| | | GGTTGACCAAGACTGAGGAGCGCACCAGAAAGATGGAGGAGGAGCCACTCGGTCCTGATCTTGAA |
| | | GATCTGAAACGACAAGTTCAGCAGCACAAGGTCCTGCAGGAGGATCTCGAACAGGAGCAGGTCCG |
| | | GGTGAACAGCCTGACCCATATGGTTGTCGTTGTCGACGAGTCAAGTGGGGACCATGCTACGGCTGC |
| | | GCTTGAGGAACAGCTGAAGGTCTTGGGCGATAGATGGGCGAATATCTGCCGCTGGACTGAAGATAG |
| | | ATGGGTGCTGCTGCAGGATATTCTTTTGAAGTGGCAGCGGTTGACCGAAGAACAATGCTTGTTTAG |
| | | CGCCTGGCTGAGTGAGAAAGAGGATGCGGTGAATAAGATACACACTACTGGCTTCAAAGACCAGA |
| | | ATGAAATGCTTAGCAGCCTGCAGAAACTTGCGGTTCTTAAGGCCGACTTGGAAAAGAAAAAGCAG |
| | | TCAATGGGTAAGCTGTACTCACTCAAGCAGGACCTGCTGTCAACTTTGAAGAACAAGAGCGTCAC |
| | | GCAAAAGACTGAGGCCTGGCTCGACAATTTTGCTAGGTGCTGGGATAACCTGGTGCAGAAGTTGG |
| | | AAAAGTCCACTGCTCAGATCTCTCAAGCCGTGACTACTACCCAGCCCAGCCTGACACAGACTACCG |
| | | TCATGGAAACCGTTACCACTGTGACGACTAGGGAGCAGATACTCGTTAAGCACGCTCAAGAGGAAT |
| | | TGCCGCCACCTCCTCCCCAAAAGAAACGCCAGATTACCGTCGACTCTGAAATACGCAAGAGACTGG |
| | | ATGTGGACATCACGGAACTGCACTCTTGGATTACCAGAAGCGAGGCAGTCCTCCAGAGCCCCGAGT |
| | | TTGCGATCTTCAGGAAGGAAGGCAACTTTTCAGATCTGAAGGAAAAGGTGAACGCCATCGAGCGC |
| | | GAGAAAGCCGAGAAGTTCCGCAAACTGCAGGATGCGAGCCGGTCTGCTCAGGCCTTGGTGGAGCA |
| | | AATGGTGAATGAAGGCGTAAATGCGGACAGCATCAAGCAGGCCTCCGAGCAGCTCAATTCAAGATG |
| | | GATCGAGTTCTGTCAGCTGCTCAGCGAGCGATTGAACTGGCTGGAGTATCAAAATAACATAATCGCG |
| | | TTTTACAACCAGCTGCAACAGCTGGAACAGATGACGACTACTGCAGAGAACTGGCTTAAAATCCAG |
| | | CCTACAACTCCTTCTGAACCCACAGCAATCAAAAGCCAGCTCAAGATCTGCAAGGACGAAGTCAA |
| | | CCGCCTCTCCGATTTGCAGCCTCAGATAGAACGACTCAAGATCCAAAGTATCGCACTCAAAGAAAA |
| | | GGGGCAAGGCCCTATGTTTCTCGATGCTGATTTTGTGGCTTTCACCAACCATTTTAAGCAAGTGTTT |
| | | TCAGATGTGCAAGCCCGGGAAAAGGAACTGCAAACCATTTTTGATACCCTGCCTCCAATGCGGTAC |
| | | CAGGAGACTATGTCTGCAATTCGCACCTGGGTGCAACAAAGCGAGACGAAACTTTCTATTCCACAA |
| | | CTGTCCGTGACAGACTACGAAATTATGGAACAGCGACTGGGAGAACTCCAAGCCCTTCAGTCCAGC |
| | | CTTCAGGAGCAGCAGTCAGGATTGTACTATCTGTCAACCACCGTGAAGGAAATGTCAAAAAAGGCT |
| | | CCCTCCGAAATATCTCGCAAGTATCAGTCTGAGTTTGAAGAGATTGAAGGGAGATGGAAGAAACTG |
| | | TCCTCACAGCTGGTTGAGCACTGTCAGAAGTTGGAAGAGCAGATGAACAAGTTGCGCAAGATTCA |
| | | AAATCACATACAGACACTCAAAAAGTGGATGGCGGAGGTTGACGTCTTCCTTAAAGAGGAGTGGC |
| | | CAGCCCTGGGCGACTCAGAGATCCTGAAAAAGCAACTGAAACAGTGCCGACTGCTGGTGTCCGAC |
| | | ATCCAGACAATACAGCCAAGTTTGAATAGCGTGAATGAGGGTGGACAAAAAATCAAAAACGAGGC |
| | | TGAGCCCGAGTTCGCCTCCAGATTGGAAACCGAGCTGAAGGAACTTAATACACAGTGGGATCATAT |
| | | GTGTCAGCAGGTTTATGCCCGGAAGGAAGCACTGAAAGGGGGGCTGGAGAAAACCGTGAGCCTTC |
| | | AGAAGGACCTTAGCGAGATGCACGAGTGGATGACCCAGGCGGAGGAAGAGTATCTCGAACGGGAT |
| | | TTTGAGTATAAGACACCAGATGAACTTCAGAAGGCAGTCGAAGAGATGAAACGGGCAAAGGAAGA |
| | | GGCTCAGCAGAAGGAAGCAAAGGTTAAACTTCTGACAGAATCCGTTAACAGCGTTATAGCCCAAG |
| | | CTCCCCCCGTTGCCCAGGAAGCTCTTAAAAAGGAACTGGAAACACTGACAACAAACTACCAGTGG |
| | | CTGTGCACCCGCTTGAACGGCAAATGCAAAACCCTGGAAGAGGTCTGGGCTTGTTGGCATGAACT |
| | | CCTGTCATACCTGGAAAAAGCTAATAAGTGGCTGAATGAGGTGGAGTTTAAATTGAAAACGACCGA |
| | | AAACATCCCTGGCGGTGCAGAGGAGATATCTGAGGTGCTCGATAGCCTGGAGAATCTTATGAGGCA |
| | | TAGCGAGGATAACCCCAACCAGATTAGGATACTGGCCCAAACTCTGACCGACGGCGGAGTGATGGA |
| | | CGAGCTGATAAACGAGGAACTGGAAACCTTTAATTCACGCTGGCGAGAACTTCACGAGGAGGCAG |
| | | TCAGGCGCCAGAAACTGCTGGAGCAGTCCATCCAGAGTGCCCAGGAGACTGAGAAGTCACTTCAT |
| | | CTGATCCAGGAGAGTTTGACGTTTATCGACAAGCAGCTTGCCGCCTACATTGCAGACAAGGTCGAT |
| | | GCTGCTCAGATGCCGCAAGAGGCTCAGAAAATCCAGAGCGACCTGACAAGCCACGAGATTAGCCT |
| | | GGAGGAAATGAAAAAGCACAACCAGGGTAAAGAGGCCGCCCAGCGGGTTCTGAGCCAGATCGAT |
| | | GTCGCACAGAAGAAACTCCAAGATGTAAGCATGAAGTTCCGACTGTTTCAGAAACCAGCGAACTT |
| | | TGAGCAGCGACTGCAGGAGTCTAAAATGATCCTCGATGAGGTGAAAATGCATCTGCCAGCCCTGGA |
| | | AACTAAGAGCGTGGAGCAGGAGGTGGTTCAGAGTCAGCTGAACCACTGCGTAAACCTCTATAAAA |
| | | GTCTTAGTGAAGTGAAGAGCGAGGTGGAGATGGTGATTAAGACTGGTAGACAGATTGTACAGAAA |
| | | AAGCAGACGGAGAACCCCAAAGAGTTGGATGAACGGGTGACAGCCCTTAAGCTCCACTATAACGA |
| | | GCTGGGAGCCAAAGTGACTGAGAGAAAGCAGCAACTTGAAAAATGTCTGAAGCTGAGCAGAAAG |
| | | ATGCGGAAGGAAATGAATGTGCTGACAGAGTGGCTGGCTGCGACCGACATGGAACTTACAAAGAG |
| | | GAGCGCCGTGGAAGGCATGCCTTCCAATCTGGATAGTGAGGTCGCTTGGGGCAAAGCTACACAGA |
| | | AGGAAATTGAGAAGCAAAAAGTGCATCTTAAGAGCATTACTGAAGTTGGCGAGGCTTTGAAAACC |
| | | GTCCTCGGCAAGAAAGAGACTCTGGTAGAAGATAAACTCTCACTGCTGAACTCAAACTGGATCGC |
| | | AGTCACTAGTCGGGCCGAAGAGTGGCTGAATTTGCTGTTGGAGTACCAAAAGCATATGGAGACTTT |
| | | CGATCAGAATGTCGACCACATTACGAAGTGGATAATCCAGGCGGACACACTGTTGGATGAGTCCGA |
| | | GAAAAAGAAACCTCAGCAGAAGGAAGACGTGCTCAAACGACTCAAAGCCGAACTGAACGACATC |
| | | AGGCCCAAAGTTGATTCCACCCGGGATCAAGCGGCCAATCTCATGGCCAACCGGGGGGATCACTGC |
| | | AGAAAGTTGGTAGAACCTCAGATAAGCGAACTTAATCATAGATTTGCCGCCATATCACATAGGATTA |
| | | AGACCGGTAAGGCTTCAATCCCCCTGAAGGAACTCGAACAGTTTAATAGTGACATCCAGAAGCTCC |
| | | TGGAGCCTCTCGAAGCCGAGATTCAACAGGGTGTAAACCTTAAGGAAGAGGATTTTAATAAGGATA |
| | | TGAATGAGGACAATGAAGGCACGGTCAAAGAGCTCCTCCAGAGGGGAGACAATTTGCAGCAGCGG |
| | | ATCACCGATGAACGCAAACGCGAAGAGATCAAAATTAAGCAGCAGCTCCTCCAAACAAAGCACAA |
| | | TGCCCTTAAAGACCTCAGAAGCCAGCGGCGGAAAAAGGCTCTGGAAATCAGCCATCAATGGTATCA |
| | | GTACAAGCGGCAAGCGGACGATCTCCTGAAATGTCTGGATGATATCGAGAAAAAACTGGCCAGTCT |
| | | GCCAGAGCCCAGAGATGAGCGCAAAATCAAGGAAATCGACAGGGAGCTTCAGAAGAAAAAAGAG |
| | | GAGCTGAACGCCGTCCGAAGGCAGGCCGAAGGGCTGTCAGAAGATGGGGCCGCCATGGCAGTGG |
| | | AACCTACCCAGATTCAACTGTCCAAGCGCTGGAGAGAGATTGAAAGTAAGTTCGCACAATTCCGGA |
| | | GGCTTAATTTTGCTCAGATTCACACAGTTAGGGAGGAAACCATGATGGTGATGACCGAAGACATGC |
| | | CACTCGAAATCAGCTACGTCCCTAGCACATATCTCACGGAGATTACCCACGTGTCCCAGGCACTGCT |
| | | GGAAGTCGAGCAATTGTTGAACGCCCCTGATCTCTGCGCCAAGGATTTCGAAGACTTGTTCAAGCA |
| | | AGAGGAAAGCCTCAAGAACATCAAAGACAGTCTTCAGCAGTCCTCAGGCAGAATTGATATTATCCA |
| | | CTCCAAGAAAACAGCCGCTCTCCAGAGCGCTACCCCTGTCGAACGAGTGAAACTGCAAGAGGCCT |
| | | TGTCTCAGTTGGACTTCCAGTGGGAGAAAGTCAACAAGATGTACAAGGACAGGCAGGGTCGGTTC |
| | | GACCGGAGCGTCGAAAAATGGCGGAGATTCCATTATGACATCAAGATATTCAATCAGTGGCTTACAG |
| | | AGGCGGAGCAATTTTTGAGAAAGACGCAGATTCCCGAAAACTGGGAGCATGCCAAGTACAAGTGG |
| | | TACCTCAAAGAGCTCCAGGATGGAATCGGCCAGAGACAGACCGTAGTTAGAACACTGAACGCAAC |
| | | TGGAGAAGAGATTATCCAGCAGTCCTCCAAAACTGACGCCTCTATCCTCCAGGAGAAGCTTGGCAG |
| | | CCTTAACCTGCGGTGGCAGGAGGTGTGTAAACAACTGTCAGATAGGAAAAAAAGACTTGAGGAAC |
| | | AGAAGAACATTCTGAGCGAGTTCCAACGAGATCTCAACGAGTTCGTACTGTGGCTGGAGGAGGCC |
| | | GACAACATCGCTTCAATCCCCCTGGAACCCGGCAAGGAACAGCAGCTTAAGGAAAAATTGGAACA |
| | | GGTAAAACTGTTGGTAGAGGAGCTCCCATTGCGGCAGGGCATTCTCAAGCAGCTGAATGAAACTGG |
| | | AGGTCCCGTTCTGGTCAGTGCGCCCATAAGCCCGGAAGAGCAGGACAAGTTGGAAAACAAGCTGA |
| | | AGCAAACAAATCTGCAGTGGATCAAAGTGAGCCGGGCTCTTCCAGAAAAGCAAGGCGAGATTGAA |
| | | GCCCAGATTAAGGATCTTGGCCAGCTTGAGAAAAAGCTGGAGGATCTGGAAGAGCAGCTGAACCA |
| | | TTTGCTTCTGTGGCTGAGTCCCATTAGAAACCAGCTGGAAATCTACAACCAGCCAAACCAAGAGGG |
| | | CCCTTTCGACGTCAAGGAAACTGAGATAGCAGTCCAGGCAAAACAGCCCGATGTCGAAGAAATCT |
| | | TGTCTAAGGGGCAGCACCTCTACAAAGAGAAACCTGCCACCCAGCCCGTGAAGAGAAAACTGGAA |
| | | GACCTCTCTTCAGAGTGGAAGGCTGTAAACAGGTTGCTCCAGGAGCTGAGAGCTAAGCAACCTGA |
| | | CCTCGCACCCGGCCTGACTACTATTGGCGCCAGCCCGACCCAAACCGTCACACTGGTGACCCAGCC |
| | | TGTAGTCACAAAAGAAACCGCCATCTCCAAACTGGAGATGCCTTCAAGCCTGATGCTGGAGGTGCC |
| | | TGCTCTGGCCGATTTCAACAGAGCCTGGACAGAGCTGACAGACTGGCTGTCTCTGCTCGACCAAGT |
| | | GATAAAGTCACAGCGCGTCATGGTGGGTGACCTGGAGGACATCAATGAGATGATCATAAAGCAAAA |
| | | GGCTACCATGCAGGACTTGGAGCAGCGCAGGCCGCAGTTGGAAGAACTGATTACAGCAGCCCAGA |
| | | ACCTCAAGAACAAGACTTCAAATCAGGAGGCAAGGACCATCATAACAGATAGAATCGAGAGGATT |
| | | CAGAACCAATGGGATGAGGTGCAGGAGCACCTGCAGAACAGAAGGCAGCAGCTGAACGAAATGC |
| | | TGAAAGACTCCACACAGTGGCTGGAGGCCAAAGAGGAGGCAGAACAGGTGCTGGGCCAGGCTAG |
| | | GGCCAAGCTGGAAAGTTGGAAGGAAGGGCCCTACACCGTGGACGCCATACAGAAGAAAATAACGG |
| | | AGACAAAACAGCTGGCCAAAGACCTCCGCCAATGGCAGACCAACGTAGACGTCGCCAATGACCTG |
| | | GCACTCAAGTTGTTGAGAGATTATTCCGCAGATGACACCCGAAAAGTACACATGATCACTGAGAAT |
| | | ATCAACGCCAGTTGGCGGAGCATTCACAAACGGGTGAGCGAGAGAGAAGCCGCCCTTGAGGAGA |
| | | CGCATCGCCTTTTGCAGCAATTTCCGCTTGACCTGGAAAAGTTTCTGGCTTGGTTGACAGAGGCCG |
| | | AGACAACTGCTAACGTGCTGCAAGATGCCACCCGCAAAGAGCGGCTTCTTGAGGACTCTAAAGGG |
| | | GTGAAAGAATTGATGAAGCAGTGGCAGGACCTCCAGGGCGAAATCGAGGCCCATACTGATGTCTAC |
| | | CACAACCTGGATGAAAATAGTCAGAAAATCCTGAGATCCCTGGAAGGGAGTGATGATGCGGTGCTG |
| | | CTGCAGCGCCGACTCGATAACATGAACTTTAAGTGGTCTGAACTCCGGAAAAAAAGCCTGAACATT |
| | | CGGAGCCATCTGGAGGCTTCTTCTGATCAGTGGAAGAGACTTCATCTGTCCTTGCAGGAGCTTCTG |
| | | GTGTGGCTGCAGCTGAAAGACGACGAGCTGTCAAGGCAGGCACCAATAGGGGGCGACTTCCCCGC |
| | | TGTTCAGAAACAAAATGATGTGCACAGGGCATTCAAGCGGGAACTGAAGACGAAAGAACCAGTGA |
| | | TTATGAGCACTCTGGAGACGGTACGCATCTTTCTGACCGAGCAGCCACTTGAAGGCCTCGAAAAGT |
| | | TGTACCAGGAGCCACGGGAATTGCCTCCCGAAGAACGGGCTCAGAATGTTACCCGACTGCTTCGCA |
| | | AGCAGGCTGAGGAAGTGAACACTGAGTGGGAAAAACTGAATCTGCACAGTGCCGATTGGCAGCGC |
| | | AAAATTGACGAGACATTGGAGCGCTTGCGGGAGCTGCAGGAAGCCACAGATGAACTTGATCTGAA |
| | | ACTCAGACAGGCCGAGGTCATAAAGGGTTCTTGGCAACCCGTAGGCGACCTTTTGATTGACTCACT |
| | | GCAGGATCATCTGGAGAAAGTAAAGGCGCTGCGGGGGGAAATTGCTCCGCTGAAGGAAAACGTGA |
| | | GCCATGTGAATGATCTGGCTCGGCAATTGACTACCCTGGGTATCCAGCTGTCCCCCTACAATCTGAG |
| | | CACTCTCGAAGACCTCAACACAAGATGGAAACTGCTGCAGGTCGCCGTGGAGGACAGGGTGAGGC |
| | | AGCTGCATGAGGCACACAGGGACTTCGGTCCGGCAAGCCAACACTTCCTGTCTACTTCTGTTCAAG |
| | | GGCCTTGGGAACGCGCTATTTCCCCAAACAAAGTGCCGTACTATATTAACCATGAAACTCAGACAAC |
| | | CTGCTGGGATCATCCTAAAATGACTGAGCTGTACCAGAGCCTCGCCGATCTGAATAACGTGAGGTTT |
| | | AGCGCATACAGAACCGCAATGAAGCTCAGGAGACTGCAGAAGGCACTTTGCCTGGACCTCCTGAG |
| | | CCTCTCCGCTGCCTGCGATGCCCTCGACCAGCACAACCTGAAGCAGAATGATCAGCCTATGGACAT |
| | | CCTGCAAATCATAAATTGTCTGACGACAATCTACGACCGGCTCGAACAGGAGCACAATAATCTCGTG |
| | | AACGTTCCACTTTGTGTGGATATGTGTCTCAACTGGCTGTTGAACGTCTATGACACCGGGAGAACTG |
| | | GAAGGATCCGCGTTCTGTCCTTTAAGACTGGAATCATCAGCTTGTGTAAGGCGCACCTTGAGGACA |
| | | AATATCGATACCTGTTTAAGCAGGTTGCAAGCTCCACTGGATTCTGCGACCAGAGGCGCCTCGGGCT |
| | | GCTGCTTCATGATAGTATCCAAATCCCACGCCAACTCGGAGAGGTGGCAAGCTTCGGCGGGAGCAA |
| | | TATTGAGCCAAGCGTCAGATCTTGTTTCCAGTTTGCCAACAATAAGCCTGAGATCGAAGCCGCACT |
| | | GTTTTTGGATTGGATGCGGTTGGAGCCCCAGAGTATGGTGTGGTTGCCCGTGCTGCATAGGGTCGCT |
| | | GCCGCTGAAACCGCCAAACACCAGGCCAAGTGCAATATTTGCAAGGAATGTCCCATCATCGGGTTT |
| | | AGATACCGGAGCCTGAAGCACTTCAATTATGATATCTGCCAGTCTTGTTTTTTTTCCGGCCGAGTTGC |
| | | TAAGGGACACAAGATGCATTACCCAATGGTGGAATATTGTACGCCAACCACCAGCGGCGAAGATGT |
| | | CAGAGATTTTGCAAAAGTACTCAAGAACAAGTTCCGCACCAAGCGATATTTCGCGAAGCACCCCCG |
| | | GATGGGCTACCTCCCAGTGCAGACGGTTCTGGAAGGCGATAACATGGAAACTCCAGTCACCCTGAT |
| | | CAATTTCTGGCCTGTGGACTCTGCGCCTGCCTCTTCACCTCAGCTCTCCCACGATGACACCCATTCC |
| | | AGAATCGAGCACTACGCGTCTCGACTGGCGGAGATGGAGAACAGCAATGGTTCTTACCTCAATGAC |
| | | AGCATTAGCCCCAACGAAAGCATCGATGACGAGCACCTCTTGATTCAACACTACTGCCAGTCCCTG |
| | | AATCAGGACAGTCCTCTGTCCCAACCCAGGAGCCCAGCCCAGATACTGATCTCTCTCGAATCAGAA |
| | | GAAAGAGGGGAGTTGGAGCGAATTTTGGCCGACCTGGAGGAGGAGAATAGAAATCTTCAGGCCGA |
| | | GTACGATAGACTGAAGCAGCAGCACGAGCACAAGGGCCTTTCTCCACTGCCCTCTCCACCGGAAAT |
| | | GATGCCAACCTCCCCTCAAAGTCCCAGGGACGCTGAGCTCATTGCCGAGGCAAAGCTGTTGCGCC |
| | | AGCACAAAGGACGCCTGGAGGCGAGAATGCAGATCCTGGAAGACCATAACAAGCAGCTTGAGTCT |
| | | CAGCTGCACCGACTTAGACAGCTGTTGGAGCAGCCACAAGCGGAAGCAAAGGTCAATGGAACTAC |
| | | CGTGAGTTCCCCCAGTACAAGTCTGCAGCGCTCTGACTCAAGCCAGCCAATGCTGCTCCGGGTAGT |
| | | CGGTTCTCAGACTAGTGATTCAATGGGGGAGGAGGATCTTCTTTCCCCACCCCAAGACACCTCAAC |
| | | AGGGCTTGAGGAAGTGATGGAGCAGCTGAACAACTCCTTCCCCTCTTCACGGGGGAGAAATACTC |
| | | CTGGGAAGCCTATGAGAGAGGATACCATGTAATGACTCGAGAATCAACCTCTGGATTACAAAATTTG |
| | | TGAAAGATTGACTGGTATTCTTAACTATGTTGCTCCTTTTACGCTATGTGGATACGCTGCTTTAATGCC |
| | | TTTGTATCATGCTATTGCTTCCCGTATGGCTTTCATTTTCTCCTCCTTGTATAAATCCTGGTTGCTGTCT |
| | | CTTTATGAGGAGTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCGCTTTCCCCCTCC |
| | | CTATTGCCACGGCGGAACTCATCGCCGCCTGCCTTGCCCGCTGCTGGACAGGGGCTCGGCTGTTGG |
| | | GCACTGACAATTCCGTGGGGTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAAATGAGGAAATTGCATCGCA |
| | | TTGTCTGAGTAGGTGTCATTCTATTCTGGGGGGTGGGGTGGGGCAGGACAGCAAGGGGGAGGATTG |
| | | GGAAGACAATAGCAGGCATGCTGGGGATGCGGTGGGCTCTATGGGAAGATGTCTACTGAGCTGTGC |
| | | GATCCCTGCTGGGGACTTTCCGCTGGGGACTTTCCGCTGGGGACTTTCCGCCTTCAGCTAAGGAAG |
| | | CTACCAATATTTAGAGGTACATTTTGTTCTAGAACAAAATGTACCGGTACATTTTGTTCTGGTACATTT |
| | | TGTTCT |
| 25. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 1-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTG |
| | | TGGTGGGAAGAGGTGGAGGATTGCTACGAGCGGGAGGATGTGCAGAAAAAGACTTTCACCAAGTG |
| | | GGTAAATGCTCAGTTCTCTAAGTTCGGGAAGCAACACATCGAGAACTTGTTTTCCGACCTCCAGGA |
| | | TGGCAGGCGCCTGCTTGACTTGCTGGAGGGTCTGACTGGTCAGAAGCTCCCTAAGGAGAAGGGCA |
| | | GCACCAGAGTTCACGCTCTGAATAACGTTAACAAAGCCCTGAGGGTCCTCCAGAATAACAATGTAG |
| | | ACCTGGTGAACATCGGTTCTACCGACATCGTGGACGGGAATCATAAACTGACCTTGGGGCTCATCT |
| | | GGAACATTATCTTGCACTGGCAGGTGAAGAACGTCATGAAGAATATCATGGCCGGGCTGCAACAGA |
| | | CCAACTCCGAAAAGATTTTGCTCAGCTGGGTGCGCCAGTCCACCCGCAATTATCCACAAGTCAACG |
| | | TTATCAATTTCACCACTAGTTGGAGTGATGGCCTCGCCCTGAACGCCTTGATCCACAGTCACCGCCC |
| | | CGACCTGTTTGACTGGAACTCTGTGGTTTGCCAACAGAGCGCTACCCAGCGTCTCGAACATGCTTT |
| | | CAATATCGCTAGATACCAGCTCGGTATCGAGAAGCTGTTGGATCCTGAGGACGTAGACACGACCTAC |
| | | CCCGATAAAAAGAGCATCCTGATGTATATCACTTCTCTGTTTCAGGTGCTTCCTCAACAGGTGTCCAT |
| | | TGAAGCCATCCAAGAGGTCGAAATGCTGCCCAGGCCCCCTAAGGTGACAAAAGAAGAGCATTTCC |
| | | AGCTGCATCACCAGATGCATTACAGTCAACAGATTACCGTCAGCTTGGCTCAGGGATATGAGCGCAC |
| | | GTCCTCTCCTAAGCCCAGATTTAAGTCCTACGCATATACCCAGGCTGCCTACGTGACCACTTCCGAC |
| | | CCAACTAGGAGCCCCTTTCCCTCCCAACACCTGGAGGCCCCTGAGGACAAAAGCTTCGGATCTAGT |
| | | CTGATGGAGTCCGAGGTGAATCTGGATCGGTACCAAACTGCTCTGGAGGAAGTGCTGTCCTGGCTT |
| | | CTGTCCGCTGAAGACACCCTGCAGGCGCAAGGTGAGATTTCTAACGACGTCGAGGTCGTGAAGGA |
| | | TCAGTTCCACACTCATGAGGGCTACATGATGGACTTGACAGCTCACCAGGGAAGAGTTGGTAACAT |
| | | CCTGCAGCTTGGCTCCAAGTTGATTGGCACGGGAAAGCTTAGCGAGGACGAGGAAACCGAGGTAC |
| | | AGGAGCAGATGAACTTGCTGAATTCCCGCTGGGAGTGCCTGAGAGTTGCGTCCATGGAAAAGCAG |
| | | TCAAACCTTCACAGAGTGCTGATGGACCTTCAGAACCAGAAGCTGAAGGAGCTGAACGATTGGCT |
| | | GACAAAGACCGAGGAACGCACAAGAAAAATGGAAGAGGAACCTCTGGGACCTGATCTGGAGGAT |
| | | CTGAAGCGCCAGGTCCAACAGCATAAGGTGCTGCAGGAGGATTTGGAGCAGGAGCAGGTGCGGGT |
| | | CAACAGCCTGACACATATGGTGGTTGTGGTCGATGAGTCCTCTGGCGATCATGCTACCGCAGCGCTG |
| | | GAAGAGCAGCTGAAGGTGCTGGGGGATCGCTGGGCCAACATTTGCAGATGGACAGAAGACCGGTG |
| | | GGTCCTGCTTCAAGATATCCTGCTCAAGTGGCAGCGCCTGACTGAAGAGCAGTGCCTGTTCTCAGC |
| | | TTGGTTGAGCGAAAAAGAGGACGCAGTTAACAAGATCCATACCACAGGCTTCAAGGATCAGAATG |
| | | AGATGCTGAGCTCACTGCAGAAGCTGGCTGTGCTGAAGGCCGACTTGGAGAAGAAAAAGCAGTCC |
| | | ATGGGCAAACTGTATAGCCTGAAGCAGGACCTGCTCAGCACCCTGAAGAACAAAAGCGTTACGCA |
| | | GAAAACTGAGGCCTGGTTGGACAACTTTGCTCGCTGCTGGGATAACCTTGTGCAGAAGCTGGAAA |
| | | AGTCAACCGCACAGATCTCCCAGGCTGTGACCACTACGCAGCCTAGCTTGACCCAGACAACCGTTA |
| | | TGGAAACCGTGACAACCGTGACAACGAGAGAGCAAATCCTGGTTAAGCATGCCCAGGAGGAACTG |
| | | CCGCCACCCCCGCCTCAGAAGAAACGGCAGATTACGGTGGACAGCGAGATCCGCAAGAGGCTGGA |
| | | CGTCGACATCACTGAGCTCCACTCATGGATCACGCGGAGTGAGGCCGTGCTGCAGTCTCCCGAGTT |
| | | CGCTATTTTCCGCAAGGAGGGAAACTTCTCTGACTTGAAAGAGAAGGTGAATGCCATCGAGCGTGA |
| | | AAAGGCAGAAAAGTTCAGAAAGCTGCAGGACGCCTCTCGCTCTGCGCAGGCCCTGGTGGAGCAG |
| | | ATGGTCAACGAGGGTGTTAACGCTGACAGCATCAAACAGGCAAGCGAACAGCTGAACTCCAGATG |
| | | GATCGAATTTTGCCAACTGCTCTCCGAACGCCTCAACTGGTTGGAGTACCAGAACAATATTATCGCT |
| | | TTTTACAACCAGCTGCAGCAACTGGAGCAGATGACAACTACCGCCGAAAACTGGCTGAAAATTCA |
| | | GCCAACCACACCGTCCGAGCCAACTGCGATCAAGTCTCAGCTGAAGATCTGCAAGGACGAAGTGA |
| | | ACCGTCTTAGCGACTTGCAACCCCAGATTGAACGGCTCAAGATTCAGTCTATTGCTCTGAAGGAGA |
| | | AGGGTCAGGGTCCGATGTTCCTGGATGCAGATTTTGTCGCATTTACCAATCACTTCAAACAGGTGTT |
| | | TAGTGACGTCCAGGCGCGCGAGAAAGAGCTGCAGACTATTTTTGACACCCTGCCTCCCATGAGGTA |
| | | CCAGGAGACAATGAGTGCCATTCGCACCTGGGTGCAACAGTCCGAGACGAAATTGTCAATCCCAC |
| | | AGCTGTCCGTGACAGACTACGAGATTATGGAGCAACGGTTGGGGGAGCTGCAGGCTCTTCAGAGTT |
| | | CCCTGCAGGAGCAACAGAGCGGACTGTACTATCTTTCCACTACCGTGAAGGAAATGAGTAAGAAA |
| | | GCCCCTTCCGAGATCAGCCGCAAATACCAGTCTGAATTCGAAGAGATCGAGGGACGCTGGAAAAA |
| | | GCTGTCAAGCCAGCTGGTCGAGCACTGTCAGAAGCTGGAAGAGCAGATGAACAAGCTGCGTAAAA |
| | | TCCAGAACCATATTCAGACCCTCAAGAAATGGATGGCAGAAGTTGACGTGTTTCTGAAGGAAGAGT |
| | | GGCCTGCCCTCGGCGATAGCGAAATCCTTAAGAAACAACTCAAGCAGTGCCGCCTCCTGGTGTCCG |
| | | ATATTCAGACAATCCAGCCTTCCCTGAACAGCGTTAATGAGGGCGGACAGAAAATTAAAAACGAGG |
| | | CCGAACCCGAGTTCGCTTCTCGCCTGGAGACTGAACTGAAGGAGCTGAATACCCAGTGGGACCATA |
| | | TGTGTCAACAGGTCTACGCACGCAAAGAGGCACTGAAGGGCGGTCTGGAGAAGACTGTCTCACTC |
| | | CAGAAGGACCTGAGCGAGATGCACGAGTGGATGACCCAGGCCGAAGAGGAATACCTGGAGCGTG |
| | | ATTTTGAGTATAAGACCCCAGACGAACTCCAGAAAGCCGTTGAAGAGATGAAGAGGGCTAAGGAG |
| | | GAAGCCCAACAGAAGGAGGCGAAAGTGAAGCTCCTGACAGAGTCCGTGAACTCCGTCATCGCTCA |
| | | AGCTCCTCCCGTGGCTCAGGAGGCCCTCAAGAAAGAGCTGGAAACTCTGACCACAAATTACCAGT |
| | | GGTTGTGTACCCGCTTGAACGGTAAGTGCAAGACCCTGGAAGAGGTGTGGGCCTGCTGGCACGAG |
| | | CTCCTGTCCTATCTTGAGAAGGCGAACAAATGGCTGAACGAAGTGGAATTCAAGTTGAAAACAACT |
| | | GAGAACATTCCGGGAGGCGCTGAGGAAATCTCTGAGGTGCTGGACTCTCTGGAAAATCTGATGCGT |
| | | CATTCTGAAGACAATCCTAACCAGATCAGGATTCTGGCACAGACCCTCACTGACGGAGGCGTTATG |
| | | GACGAACTTATCAACGAGGAACTGGAGACATTCAATTCCCGTTGGCGTGAGCTGCACGAAGAGGC |
| | | CGTGCGCCGTCAGAAGTTGCTGGAACAGTCTATCCAGTCCGCCCAGGAGACAGAGAAATCTCTCCA |
| | | CCTGATCCAGGAGAGTCTGACGTTTATCGATAAGCAGCTGGCTGCCTACATCGCCGACAAGGTGGA |
| | | CGCTGCCCAGATGCCCCAAGAGGCTCAGAAGATCCAGTCCGACCTGACGTCTCATGAAATCAGCCT |
| | | GGAAGAGATGAAAAAGCACAACCAGGGCAAAGAGGCTGCCCAGCGCGTCCTCTCCCAGATTGAC |
| | | GTGGCTCAGAAAAAGTTGCAGGACGTGAGCATGAAGTTTAGGCTGTTCCAGAAACCCGCCAACTT |
| | | CGAACAGAGACTGCAGGAGTCTAAGATGATCCTGGATGAGGTCAAGATGCACCTCCCTGCTCTGGA |
| | | AACAAAATCTGTGGAGCAGGAGGTCGTGCAGTCACAACTGAACCATTGTGTGAACCTCTACAAGT |
| | | CTCTCAGCGAGGTGAAGTCCGAGGTCGAGATGGTGATCAAGACCGGACGCCAGATTGTGCAGAAA |
| | | AAGCAGACAGAGAACCCAAAGGAGCTGGACGAACGGGTGACCGCCCTGAAGCTCCACTACAACG |
| | | AGTTGGGTGCCAAAGTCACTGAAAGAAAGCAACAGCTGGAGAAGTGTCTGAAGCTGAGCCGCAA |
| | | GATGCGCAAAGAAATGAACGTCTTGACCGAGTGGTTGGCGGCCACCGACATGGAGCTGACCAAGA |
| | | GATCCGCTGTGGAGGGCATGCCCTCCAACCTTGACTCAGAGGTGGCCTGGGGCAAGGCCACACAG |
| | | AAGGAGATCGAAAAACAGAAGGTGCATCTTAAATCAATCACTGAGGTGGGCGAAGCTCTGAAGAC |
| | | AGTGCTGGGCAAGAAAGAGACACTTGTAGAGGACAAGCTCTCCCTTCTGAATAGCAATTGGATCGC |
| | | CGTAACTAGTCGCGCGGAGGAATGGCTGAACCTCCTGTTGGAATACCAGAAACACATGGAAACCTT |
| | | CGATCAGAACGTGGACCATATTACCAAGTGGATTATCCAGGCTGACACTCTCCTGGACGAGTCTGA |
| | | GAAAAAGAAACCCCAGCAAAAGGAAGATGTGCTGAAGCGCCTGAAGGCGGAATTGAACGACATC |
| | | CGCCCGAAGGTTGATTCCACCCGCGACCAAGCGGCCAACCTTATGGCTAACCGCGGGGACCATTGC |
| | | AGAAAACTGGTGGAACCGCAGATCTCTGAGCTGAACCACAGATTCGCCGCGATTTCTCACAGAATT |
| | | AAGACTGGCAAGGCCTCCATCCCTCTGAAAGAGCTGGAGCAGTTCAACTCTGATATTCAGAAGCTT |
| | | CTGGAGCCCCTGGAGGCAGAAATCCAACAGGGCGTCAACCTGAAAGAGGAAGACTTTAATAAGGA |
| | | CATGAATGAGGATAACGAAGGTACCGTCAAGGAGCTCCTGCAGAGGGGCGACAATCTGCAGCAAA |
| | | GAATCACTGACGAACGTAAGCGCGAAGAGATTAAGATCAAGCAACAGCTGTTGCAGACTAAGCAC |
| | | AACGCCCTGAAGGATCTGAGGTCCCAGCGCAGGAAAAAGGCCCTGGAGATTTCCCACCAGTGGTA |
| | | CCAGTACAAGCGCCAGGCCGATGACCTTCTGAAGTGCCTCGACGATATCGAGAAAAAGCTGGCCTC |
| | | ACTGCCCGAGCCCCGCGACGAGCGCAAGATCAAGGAGATTGACCGTGAGCTCCAGAAGAAAAAG |
| | | GAGGAACTGAACGCGGTGCGCAGACAGGCCGAGGGCCTCTCCGAGGACGGCGCTGCGATGGCTG |
| | | TTGAGCCCACTCAGATCCAACTGAGCAAGCGGTGGAGAGAGATCGAGTCTAAGTTCGCGCAATTTA |
| | | GGCGCCTCAACTTCGCCCAGATTCACACGGTCCGTGAGGAAACGATGATGGTGATGACAGAGGATA |
| | | TGCCACTGGAAATCAGCTACGTGCCCAGTACATATCTCACCGAAATCACCCATGTGTCCCAGGCTCT |
| | | CCTGGAGGTGGAACAGTTGCTCAACGCTCCCGACTTGTGCGCTAAGGACTTCGAGGACCTGTTCAA |
| | | GCAGGAAGAGAGCCTGAAAAACATTAAGGACTCCCTGCAACAGTCAAGCGGCCGCATCGATATCAT |
| | | TCATTCTAAGAAAACCGCTGCCCTGCAGTCTGCAACCCCCGTAGAGCGCGTCAAACTGCAGGAAG |
| | | CTCTCAGCCAACTGGATTTCCAGTGGGAGAAGGTGAACAAGATGTATAAGGATCGTCAGGGCCGGT |
| | | TCGACCGCTCTGTGGAGAAGTGGCGCCGTTTCCACTACGATATCAAGATTTTCAATCAATGGTTGAC |
| | | CGAGGCAGAGCAGTTCCTCAGAAAGACACAGATCCCTGAAAATTGGGAGCATGCCAAGTACAAGT |
| | | GGTATCTGAAGGAGCTGCAGGACGGGATCGGACAACGTCAGACCGTGGTCCGCACCCTGAACGCA |
| | | ACCGGCGAGGAAATTATCCAACAGTCTAGCAAGACAGATGCTAGCATCCTCCAGGAGAAGCTCGGA |
| | | AGCCTCAACCTTCGCTGGCAGGAGGTGTGCAAGCAGCTGTCAGACAGGAAGAAACGCCTGGAGG |
| | | AACAGAAGAACATCCTTTCAGAGTTCCAGAGAGATCTGAACGAATTCGTGCTTTGGCTTGAAGAGG |
| | | CCGATAACATCGCAAGCATCCCACTGGAGCCTGGTAAGGAACAGCAATTGAAGGAGAAGCTGGAG |
| | | CAGGTAAAGCTGCTCGTGGAAGAGCTGCCTCTGAGGCAGGGAATTCTCAAGCAGCTCAACGAGAC |
| | | TGGGGGCCCGGTTTTGGTGAGCGCCCCAATTAGCCCAGAAGAGCAGGACAAGTTGGAGAACAAGC |
| | | TGAAGCAGACCAACCTGCAGTGGATCAAGGTGTCCCGCGCACTGCCCGAAAAGCAGGGAGAGATT |
| | | GAGGCCCAGATCAAGGATCTGGGACAGCTGGAGAAAAAGCTGGAAGACCTGGAGGAACAGCTGA |
| | | ACCATTTGCTCCTGTGGTTGTCCCCCATCAGAAATCAGCTGGAAATCTACAACCAACCGAACCAGG |
| | | AAGGTCCGTTTGATGTTAAGGAAACCGAGATCGCTGTGCAGGCGAAGCAGCCTGACGTAGAAGAG |
| | | ATCCTGTCAAAGGGACAGCACCTCTACAAGGAGAAGCCCGCCACCCAGCCAGTCAAGCGGAAGCT |
| | | CGAAGACCTGTCCAGCGAGTGGAAAGCCGTCAACCGCCTCCTGCAAGAGCTGCGTGCCAAGCAAC |
| | | CGGACCTGGCGCCAGGTCTGACTACCATTGGCGCAAGTCCTACCCAGACTGTCACCCTGGTGACCC |
| | | AACCCGTTGTGACAAAGGAAACCGCCATCAGCAAGCTGGAGATGCCTAGCTCTTTGATGCTGGAGG |
| | | TTCCCGCCCTCGCCGACTTCAACCGTGCTTGGACAGAACTGACCGACTGGCTGTCCCTCCTGGACC |
| | | AAGTGATCAAGAGCCAGCGCGTGATGGTGGGTGATCTTGAGGATATTAACGAAATGATTATCAAGC |
| | | AGAAGGCCACCATGCAGGACTTGGAGCAGAGGCGCCCCCAGTTGGAGGAACTGATCACCGCCGCG |
| | | CAGAATCTGAAAAACAAAACTTCCAATCAGGAAGCGAGAACCATCATTACAGACAGAATTGAGAG |
| | | AATTCAAAACCAGTGGGACGAAGTGCAAGAACACCTCCAGAACCGGCGTCAACAGCTGAACGAG |
| | | ATGCTGAAGGATTCCACTCAATGGCTGGAAGCTAAGGAAGAGGCCGAGCAGGTCCTTGGACAGGC |
| | | ACGCGCTAAGCTGGAGTCCTGGAAAGAAGGCCCCTATACAGTGGACGCAATTCAAAAGAAAATCA |
| | | CCGAGACGAAGCAACTGGCCAAGGACCTGAGACAGTGGCAAACCAACGTTGATGTGGCTAATGAC |
| | | CTGGCCTTGAAGCTCCTGCGTGACTACTCCGCGGATGACACTAGAAAGGTGCACATGATCACCGAG |
| | | AATATCAACGCTAGCTGGCGCTCCATCCATAAGCGTGTGAGTGAGCGCGAAGCCGCTCTGGAGGAA |
| | | ACCCATCGCCTGCTTCAACAGTTTCCTTTGGACCTGGAAAAGTTCCTGGCCTGGCTCACAGAAGCC |
| | | GAGACAACCGCCAACGTGCTGCAGGACGCCACCCGTAAGGAGCGCTTGCTGGAGGATAGTAAGGG |
| | | GGTGAAGGAGTTGATGAAGCAGTGGCAAGATCTGCAGGGGGAAATCGAGGCACATACCGACGTCT |
| | | ATCATAACCTGGATGAGAATTCCCAGAAAATCCTGCGCAGCTTGGAGGGCAGTGATGACGCGGTGC |
| | | TTCTGCAACGGCGCTTGGACAACATGAACTTCAAGTGGTCCGAGTTGCGTAAAAAGAGTCTGAAC |
| | | ATTCGCTCCCACCTGGAGGCTTCTAGTGACCAGTGGAAGCGCCTGCACCTGAGCCTGCAAGAACTG |
| | | CTCGTGTGGCTGCAACTGAAGGATGACGAGTTGAGTCGTCAGGCCCCTATCGGAGGTGACTTTCCG |
| | | GCTGTCCAGAAACAGAACGACGTGCATAGGGCATTTAAGCGCGAGTTGAAGACAAAGGAGCCAGT |
| | | CATTATGTCCACGCTGGAAACCGTGCGCATTTTCCTGACTGAGCAGCCTCTGGAGGGGCTGGAGAA |
| | | GCTGTACCAGGAGCCTCGCGAGTTGCCCCCTGAAGAGCGTGCCCAGAATGTGACCCGCCTGTTGCG |
| | | TAAGCAGGCCGAGGAAGTGAACACCGAGTGGGAGAAGCTGAACCTGCATTCCGCGGATTGGCAGA |
| | | GGAAGATTGACGAGACGCTGGAGAGGCTCCGTGAGTTGCAGGAGGCAACCGACGAACTGGACCT |
| | | CAAGCTGCGTCAAGCAGAGGTGATCAAGGGGAGTTGGCAGCCGGTCGGCGATTTGCTGATCGACT |
| | | CCCTGCAGGATCACCTGGAAAAGGTTAAAGCTTTGCGCGGGGAAATTGCCCCGCTGAAGGAGAAC |
| | | GTGAGCCACGTTAACGATCTGGCCAGACAGCTGACCACTCTGGGTATCCAGCTGAGCCCGTACAAT |
| | | CTGAGCACCCTCGAAGACTTGAATACGAGATGGAAACTCCTGCAGGTGGCAGTGGAGGACAGGGT |
| | | GCGCCAATTGCACGAGGCCCACCGGGACTTTGGACCGGCCAGCCAGCATTTCCTCAGTACCTCTGT |
| | | TCAGGGCCCGTGGGAGCGGGCCATCTCCCCCAACAAGGTCCCCTATTACATCAACCATGAAACCCA |
| | | GACTACCTGTTGGGACCACCCTAAAATGACTGAGTTGTATCAATCTCTGGCCGACTTGAATAACGTG |
| | | CGCTTCTCAGCCTACAGGACTGCAATGAAACTCAGACGCCTCCAAAAGGCCCTTTGTCTGGACTTG |
| | | CTGTCCCTTAGCGCTGCCTGTGATGCTCTGGATCAGCACAATTTGAAACAAAACGACCAGCCGATG |
| | | GACATCCTCCAGATTATCAACTGCCTGACTACCATCTACGACAGACTGGAACAGGAGCATAATAACC |
| | | TGGTGAACGTCCCCCTGTGCGTGGACATGTGCCTGAACTGGCTGCTCAATGTGTATGACACGGGTA |
| | | GAACAGGACGGATCAGGGTCCTGTCCTTTAAGACCGGTATCATTTCTCTGTGTAAGGCACATCTGGA |
| | | GGACAAGTACCGCTACCTGTTTAAGCAGGTCGCCAGCTCCACTGGCTTCTGCGATCAAAGGCGGCT |
| | | GGGTCTGCTCCTGCACGACTCCATTCAGATCCCGAGACAGTTGGGTGAAGTGGCGTCCTTCGGCGG |
| | | TTCTAATATCGAGCCTAGCGTCCGCAGCTGTTTCCAATTCGCCAATAACAAGCCGGAGATCGAAGCG |
| | | GCCCTTTTCCTGGACTGGATGCGTCTGGAACCTCAGAGCATGGTTTGGCTTCCGGTGCTTCATCGCG |
| | | TGGCCGCTGCCGAAACCGCTAAACATCAGGCCAAATGTAACATCTGTAAGGAATGCCCGATTATCG |
| | | GTTTCCGGTACCGTAGCCTCAAGCATTTCAACTATGATATTTGTCAATCTTGCTTCTTTAGCGGACGG |
| | | GTGGCCAAAGGCCACAAAATGCACTACCCGATGGTCGAGTATTGCACTCCTACAACCTCCGGCGAG |
| | | GACGTGCGCGATTTTGCCAAAGTGCTGAAAAACAAGTTTAGAACCAAGCGTTACTTTGCCAAGCAT |
| | | CCACGTATGGGCTACCTGCCTGTCCAGACCGTTCTGGAAGGTGATAACATGGAGACACCCGTGACT |
| | | TTGATTAACTTTTGGCCAGTAGACTCTGCTCCGGCAAGCTCCCCTCAGCTCTCCCACGATGACACTC |
| | | ACAGCCGTATCGAGCACTACGCCTCTCGCCTGGCTGAGATGGAGAACTCTAACGGTTCATACCTGA |
| | | ATGACAGTATCTCCCCAAACGAGAGTATCGATGACGAACATCTGCTCATTCAGCATTACTGTCAATC |
| | | CTTGAATCAGGACAGCCCCCTGTCCCAGCCACGGTCCCCGGCCCAGATCCTGATTTCACTGGAGTC |
| | | CGAAGAGAGAGGGGAACTGGAAAGGATCCTCGCTGACCTGGAAGAGGAAAACCGGAACCTGCAG |
| | | GCTGAATACGACCGCTTGAAGCAGCAACACGAGCACAAGGGCTTGAGCCCGTTGCCAAGCCCTCC |
| | | GGAGATGATGCCAACTAGCCCTCAGAGCCCACGGGATGCCGAGCTCATCGCTGAAGCTAAGTTGCT |
| | | GAGACAGCACAAGGGACGTCTGGAGGCCCGCATGCAGATCCTGGAGGATCATAATAAACAGTTGG |
| | | AGAGCCAACTGCATAGGCTGCGCCAGCTCCTGGAGCAGCCACAGGCCGAGGCTAAGGTCAACGGA |
| | | ACTACCGTTTCCAGCCCGTCCACATCTCTGCAGCGCTCAGATAGTTCCCAGCCTATGTTGCTGAGAG |
| | | TGGTTGGATCTCAGACCTCCGACTCAATGGGCGAAGAGGACTTGCTGTCTCCCCCACAGGACACTA |
| | | GCACAGGACTCGAAGAGGTGATGGAACAGCTCAATAACTCTTTTCCTTCTAGTCGGGGTCGCAATA |
| | | CCCCTGGAAAGCCCATGCGCGAAGACACCATGTAATAGCTCGAGAATCAACCTCTGGATTACAAAA |
| | | TTTGTGAAAGATTGACTGGTATTCTTAACTATGTTGCTCCTTTTACGCTATGTGGATACGCTGCTTTAA |
| | | TGCCTTTGTATCATGCTATTGCTTCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTGGTGTGCACTGTGTTTGCTGA |
| | | CGCAACCCCCACTGGTTGGGGCATTGCCACCACCTGTCAGCTCCTTTCCGGGACTTTCGCTTTCCCC |
| | | CTCCCTATTGCCACGGCGGAACTCATCGCCGCCTGCCTTGCCCGCTGCTGGACAGGGGCTCGGCTG |
| | | TTGGGCACTGACAATTCCGTGGGGTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAATAAAATGAGGAAATTGCAT |
| | | CGCATTGTCTGAGTAGGTGTCATTCTATTCTGGGGGGTGGGGTGGGGCAGGACAGCAAGGGGGAG |
| | | GATTGGGAAGACAATAGCAGGCATGCTGGGGATGCGGTGGGCTCTATGGGAAGATGTCTACTGAGC |
| | | TGTGCGATCCCTGCTGGGGACTTTCCGCTGGGGACTTTCCGCTGGGGACTTTCCGCCTTCAGCTAA |
| | | GGAAGCTACCAATATTTAGAGGTACATTTTGTTCTAGAACAAAATGTACCGGTACATTTTGTTCTGGT |
| | | ACATTTTGTTCT |
| 26. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 3-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTG |
| | | TGGTGGGAAGAGGTGGAGGACTGTTACGAGAGAGAGGACGTGCAGAAGAAGACATTCACCAAGT |
| | | GGGTGAACGCTCAGTTCAGCAAGTTCGGCAAGCAGCACATCGAGAACCTGTTTAGCGATCTACAA |
| | | GATGGCAGAAGACTGCTGGACCTGCTGGAGGGCCTGACCGGGCAGAAGCTGCCCAAAGAAAAGG |
| | | GGTCTACAAGAGTGCACGCCCTGAACAACGTGAACAAGGCCCTGAGAGTGCTGCAGAACAACAA |
| | | CGTGGACCTGGTGAACATCGGCAGCACCGACATCGTGGACGGCAACCACAAGCTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATCCTGCTGAGCTGGGTGAGACAGAGCACAAGAAACTACCCCCA |
| | | AGTGAACGTGATCAACTTCACCACAAGCTGGAGCGACGGCCTGGCCCTGAACGCCCTGATCCACA |
| | | GCCACAGACCCGACCTGTTCGACTGGAACAGCGTGGTGTGTCAGCAGAGCGCCACACAGAGACTG |
| | | GAGCACGCCTTCAACATCGCTAGATATCAGCTGGGCATCGAGAAGTTACTGGACCCCGAAGATGTG |
| | | GACACCACCTACCCCGACAAGAAGAGCATCCTGATGTACATCACAAGCCTGTTCCAAGTGCTGCCT |
| | | CAGCAAGTGAGCATCGAGGCCATCCAAGAAGTCGAGATGCTGCCTAGACCCCCCAAGGTGACCAA |
| | | GGAGGAGCACTTTCAGCTGCACCATCAGATGCACTACTCTCAGCAGATCACAGTCAGCTTGGCACA |
| | | AGGCTACGAGAGAACAAGCAGCCCCAAGCCTAGATTCAAGAGCTACGCCTACACCCAAGCCGCCT |
| | | ACGTGACCACAAGTGACCCCACAAGAAGCCCCTTCCCTTCTCAGCACCTGGAGGCCCCCGAGGAC |
| | | AAGAGCTTCGGCAGCAGCCTGATGGAGAGCGAGGTGAACCTGGACAGATATCAGACCGCGTTAGA |
| | | AGAAGTGTTGAGCTGGCTGCTGAGCGCCGAGGACACCCTGCAAGCCCAAGGCGAGATCAGCAAC |
| | | GATGTGGAGGTGGTGAAGGATCAGTTCCACACCCACGAGGGCTACATGATGGACCTGACCGCCCAC |
| | | CAAGGCAGAGTGGGCAACATCCTGCAGCTGGGCAGCAAGCTGATCGGCACCGGCAAGCTGAGCG |
| | | AGGACGAGGAAACCGAGGTTCAAGAGCAAATGAACCTGCTAAACAGCAGATGGGAGTGCCTGAG |
| | | AGTGGCTAGCATGGAAAAGCAGAGTAACCTGCACAGAGTGCTGATGGACCTGCAGAATCAGAAAT |
| | | TGAAAGAACTTAATGATTGGCTGACCAAGACCGAGGAGAGAACAAGAAAGATGGAGGAGGAGCC |
| | | CCTGGGCCCCGACCTGGAGGACCTGAAGAGACAAGTGCAGCAGCACAAGGTGCTGCAAGAGGAC |
| | | CTGGAGCAAGAGCAAGTGAGAGTGAACTCTCTGACACACATGGTGGTGGTAGTGGACGAGAGCAG |
| | | CGGCGATCACGCTACCGCGGCGCTGGAAGAGCAGCTGAAAGTGCTGGGCGACAGATGGGCCAACA |
| | | TCTGCAGATGGACCGAGGACAGATGGGTGCTGCTGCAAGACATCCTGCTGAAGTGGCAGAGACTG |
| | | ACCGAGGAGCAGTGCCTGTTCAGCGCCTGGCTGAGCGAGAAGGAAGACGCTGTGAACAAGATCC |
| | | ACACCACCGGCTTCAAGGATCAGAACGAGATGCTGAGCTCCCTCCAAAAGCTCGCAGTGCTGAAG |
| | | GCCGACCTGGAAAAGAAAAAACAGTCGATGGGCAAGCTGTACAGCCTGAAGCAAGACCTGCTGA |
| | | GCACCCTGAAGAACAAGAGCGTGACACAGAAGACCGAGGCCTGGCTGGACAACTTCGCTAGATGC |
| | | TGGGACAACCTGGTACAGAAGTTAGAGAAGTCTACCGCTCAGATCAGCCAAGCCGTAACCACCAC |
| | | ACAACCTAGCCTGACTCAGACCACCGTGATGGAAACCGTGACCACCGTGACAACCCGTGAGCAGA |
| | | TCCTGGTGAAGCACGCCCAAGAGGAGCTGCCCCCCCCCCCCCCTCAGAAGAAGAGACAGATCACC |
| | | GTGGACTCAGAGATTCGCAAGAGACTGGACGTGGACATCACCGAGCTGCACAGCTGGATCACAAG |
| | | AAGCGAGGCCGTGCTGCAGAGCCCTGAGTTCGCGATCTTTAGAAAGGAGGGCAACTTCAGCGACT |
| | | TGAAGGAGAAAGTGAACGCCATCGAGAGAGAGAAGGCCGAGAAGTTCAGAAAGTTGCAAGACGC |
| | | CTCAAGAAGCGCCCAAGCCCTGGTGGAGCAGATGGTTAACGAAGGCGTGAATGCCGACAGCATCA |
| | | AGCAAGCTAGCGAGCAGCTGAATAGCCGGTGGATTGAATTCTGTCAGCTGCTGAGCGAGAGATTAA |
| | | ACTGGCTGGAGTATCAGAACAACATCATCGCCTTCTACAATCAGCTGCAGCAACTAGAACAAATGA |
| | | CCACCACCGCCGAGAACTGGCTAAAGATTCAGCCCACCACCCCTAGCGAGCCCACCGCCATCAAAT |
| | | CTCAGCTGAAAATTTGCAAGGACGAAGTGAACAGACTGTCGGACCTGCAGCCTCAAATCGAAAGA |
| | | CTGAAAATTCAGAGCATCGCACTTAAGGAGAAGGGCCAAGGACCCATGTTCCTGGACGCCGACTTC |
| | | GTGGCCTTCACCAACCACTTCAAGCAAGTGTTCAGCGACGTGCAAGCTAGAGAGAAGGAGCTGCA |
| | | GACCATCTTCGACACCCTGCCCCCCATGAGATACCAAGAAACCATGAGCGCCATCAGAACCTGGGT |
| | | GCAGCAGAGCGAAACCAAGCTGAGCATCCCTCAGCTGAGCGTGACCGACTACGAGATCATGGAGC |
| | | AAAGACTGGGAGAGCTGCAAGCCCTGCAGAGCAGCCTGCAAGAGCAGCAGAGCGGCCTGTACTA |
| | | CCTGAGCACCACCGTGAAGGAGATGTCGAAGAAAGCCCCGTCGGAGATCAGCAGAAAGTATCAGA |
| | | GCGAGTTCGAGGAGATCGAGGGCAGATGGAAGAAGCTGAGCAGCCAACTGGTGGAACACTGTCA |
| | | GAAATTAGAAGAACAGATGAACAAGCTGAGAAAGATTCAGAACCACATTCAGACCCTGAAGAAGT |
| | | GGATGGCCGAGGTGGACGTGTTCCTGAAAGAGGAGTGGCCCGCCCTGGGCGATTCCGAAATCTTA |
| | | AAAAAGCAGCTGAAGCAGTGCAGACTGCTGGTGTCAGATATCCAAACCATTCAACCAAGCCTGAA |
| | | CAGCGTGAACGAAGGCGGGCAGAAGATCAAGAACGAGGCCGAGCCCGAGTTCGCTAGCAGACTG |
| | | GAAACCGAGCTGAAGGAGCTGAATACACAGTGGGACCACATGTGTCAGCAAGTGTACGCTAGAAA |
| | | GGAAGCGCTGAAGGGCGGGCTGGAGAAGACCGTTAGCCTGCAAAAGGATCTGAGCGAGATGCAC |
| | | GAGTGGATGACCCAAGCCGAGGAGGAGTACCTGGAGAGAGACTTCGAGTACAAGACCCCCGACG |
| | | AGCTGCAGAAGGCCGTCGAGGAAATGAAGAGAGCGAAGGAGGAGGCTCAGCAGAAGGAAGCCA |
| | | AGGTGAAGCTGCTGACTGAATCTGTCAACAGTGTGATAGCTCAAGCACCCCCCGTTGCCCAAGAGG |
| | | CACTAAAGAAGGAGCTGGAAACTCTGACCACCAACTATCAGTGGCTGTGCACAAGACTGAACGGC |
| | | AAGTGCAAGACCCTGGAGGAAGTGTGGGCCTGCTGGCACGAGCTGCTGAGCTACCTGGAGAAGGC |
| | | CAACAAGTGGCTGAACGAGGTGGAGTTCAAGCTGAAGACCACCGAGAACATCCCCGGCGGCGCC |
| | | GAGGAGATCAGCGAGGTGCTGGACAGCCTGGAGAACCTGATGAGACACAGCGAGGACAACCCCA |
| | | ATCAGATCAGAATCCTGGCTCAGACCCTGACCGACGGCGGCGTGATGGACGAGCTGATCAACGAG |
| | | GAGCTGGAAACCTTTAACTCTAGATGGCGAGAACTGCACGAGGAGGCCGTGAGAAGACAGAAGCT |
| | | GCTGGAGCAAAGCATTCAGAGCGCCCAAGAAACCGAGAAGAGCCTGCACCTGATCCAAGAGAGC |
| | | CTGACCTTCATCGACAAGCAGCTGGCCGCCTACATCGCCGACAAGGTGGACGCCGCTCAGATGCCA |
| | | CAAGAGGCTCAGAAGATTCAGTCCGATCTGACAAGCCACGAGATCAGCCTGGAGGAGATGAAAAA |
| | | ACACAACCAAGGCAAGGAGGCCGCTCAGAGAGTGCTGTCTCAGATCGACGTGGCTCAGAAGAAG |
| | | CTGCAAGACGTGAGCATGAAGTTCAGACTGTTTCAGAAGCCCGCCAACTTCGAGCAGCGACTGCA |
| | | AGAGAGCAAGATGATCCTGGACGAGGTGAAGATGCACCTGCCCGCCCTGGAAACCAAGAGCGTGG |
| | | AGCAAGAGGTGGTGCAGTCTCAGTTGAACCACTGCGTGAACCTGTACAAGAGCCTGTCGGAGGTC |
| | | AAGAGTGAGGTCGAGATGGTGATCAAAACTGGGAGACAGATCGTGCAAAAAAAGCAGACCGAGA |
| | | ACCCCAAGGAGCTGGACGAGCGTGTTACCGCCCTGAAGCTGCACTACAACGAGCTGGGCGCCAAG |
| | | GTGACCGAGAGAAAGCAGCAGCTGGAGAAGTGCCTGAAGCTGAGCAGAAAGATGAGAAAGGAG |
| | | ATGAACGTGCTGACCGAGTGGCTGGCCGCCACCGACATGGAGCTGACCAAGAGAAGCGCCGTGGA |
| | | GGGCATGCCTAGCAACCTGGACAGCGAGGTGGCCTGGGGCAAGGCCACACAGAAGGAGATCGAG |
| | | AAGCAGAAGGTGCACCTGAAGAGCATCACCGAGGTGGGCGAGGCCCTGAAGACCGTGCTGGGCA |
| | | AGAAGGAAACCCTGGTGGAGGACAAGCTGAGCCTGCTGAACTCGAACTGGATCGCCGTGACAAG |
| | | CAGAGCCGAGGAGTGGCTGAACCTGCTGTTAGAGTACCAAAAACACATGGAAACCTTCGATCAGA |
| | | ACGTGGACCACATCACCAAGTGGATCATCCAAGCCGACACCCTGCTGGACGAAAGCGAAAAGAAG |
| | | AAGCCGCAACAAAAGGAGGATGTGCTGAAGAGACTGAAGGCCGAACTGAACGATATCAGACCCA |
| | | AGGTGGACAGCACACGGGACCAAGCCGCCAACCTGATGGCCAACAGAGGCGACCACTGCAGAAA |
| | | ACTGGTGGAGCCTCAGATCAGCGAGCTGAACCACAGATTCGCCGCCATCAGCCACAGAATCAAAA |
| | | CCGGCAAAGCGAGCATCCCCTTGAAGGAACTGGAGCAATTTAACAGCGACATCCAAAAATTGCTCG |
| | | AACCACTGGAGGCCGAGATTCAGCAAGGCGTGAACCTGAAGGAAGAGGACTTCAACAAGGACAT |
| | | GAACGAGGACAACGAGGGCACCGTGAAAGAACTGCTGCAGAGAGGCGACAACCTGCAGCAGAG |
| | | AATCACCGACGAGAGAAAGAGAGAGGAGATCAAGATCAAGCAGCAGCTGCTGCAGACTAAGCAC |
| | | AACGCCCTGAAGGATCTGAGATCTCAGAGGAGAAAAAAGGCTCTGGAAATTTCACATCAGTGGTAT |
| | | CAGTACAAGAGACAAGCAGACGACCTGCTGAAGTGCCTGGACGACATCGAGAAGAAGTTAGCTAG |
| | | CCTGCCCGAGCCTAGAGACGAGAGAAAGATCAAGGAGATCGACAGAGAGTTACAAAAGAAGAAG |
| | | GAGGAGCTGAATGCGGTAAGAAGACAAGCGGAAGGCCTGTCCGAGGACGGCGCAGCCATGGCCG |
| | | TGGAGCCCACACAGATTCAGCTCAGCAAGAGATGGAGAGAGATCGAGAGCAAGTTCGCTCAGTTC |
| | | AGAAGACTGAACTTCGCTCAGATCCACACCGTGAGAGAGGAAACCATGATGGTGATGACCGAGGA |
| | | CATGCCCCTGGAGATCTCATACGTTCCTAGCACCTACCTGACCGAGATCACCCACGTGAGCCAAGCC |
| | | CTGCTGGAGGTGGAGCAGCTGCTGAACGCCCCCGACCTGTGCGCCAAGGACTTCGAGGACCTGTT |
| | | CAAACAAGAGGAGAGCCTGAAGAACATCAAGGACAGCCTGCAGCAAAGTAGCGGCAGAATCGAC |
| | | ATCATCCACAGCAAGAAGACCGCCGCCCTGCAGAGCGCCACCCCCGTGGAGAGAGTGAAGCTTCA |
| | | AGAGGCCCTTTCGCAGCTGGATTTTCAGTGGGAGAAGGTGAACAAAATGTACAAGGACAGACAAG |
| | | GCAGATTCGACAGAAGCGTGGAGAAGTGGAGAAGATTCCACTACGACATCAAGATCTTCAATCAGT |
| | | GGCTGACCGAGGCAGAGCAGTTCCTGAGAAAGACACAGATCCCCGAGAACTGGGAGCACGCCAA |
| | | GTACAAGTGGTACCTGAAGGAGCTGCAAGACGGCATCGGGCAGAGACAGACCGTGGTGAGAACC |
| | | CTGAACGCCACCGGCGAGGAGATCATCCAACAAAGCTCCAAAACCGACGCTAGCATCCTGCAAGA |
| | | GAAACTGGGCAGCCTGAACCTGAGATGGCAAGAGGTGTGCAAGCAGCTGAGCGACCGCAAAAAA |
| | | CGGTTAGAGGAGCAGAAGAACATCCTGAGCGAGTTTCAGCGGGACCTGAACGAGTTCGTGCTGTG |
| | | GCTGGAGGAGGCCGATAACATCGCTAGCATCCCCCTGGAGCCCGGCAAGGAGCAGCAGTTAAAAG |
| | | AAAAACTTGAGCAAGTGAAGCTGCTGGTGGAGGAGCTGCCCCTGAGACAAGGCATCCTGAAGCA |
| | | GCTGAACGAGACTGGCGGCCCCGTGCTGGTGAGCGCCCCCATCAGCCCCGAGGAGCAAGACAAGC |
| | | TGGAGAACAAGCTGAAGCAGACCAACCTGCAGTGGATCAAGGTGAGCAGAGCCCTGCCCGAGAA |
| | | GCAAGGCGAAATCGAGGCCCAAATAAAGGACCTGGGGCAGCTGGAGAAGAAACTCGAAGACTTG |
| | | GAAGAACAACTCAACCACTTGCTGCTGTGGCTGAGCCCCATCAGAAATCAGCTGGAGATCTACAAT |
| | | CAGCCCAACCAAGAGGGGCCATTCGACGTGAAGGAAACCGAGATCGCCGTGCAAGCCAAGCAGC |
| | | CCGATGTGGAGGAGATCCTGAGCAAGGGGCAGCACCTGTACAAGGAGAAGCCCGCCACACAGCCC |
| | | GTCAAGCGTAAACTAGAGGATCTTTCCTCTGAGTGGAAGGCCGTGAACCGTCTGCTACAAGAGCTG |
| | | CGGGCTAAGCAACCCGACTTAGCGCCCGGCCTGACCACCATCGGTGCAAGCCCCACGCAGACCGT |
| | | GACCCTGGTGACCCAACCCGTGGTGACCAAGGAAACCGCCATCAGCAAGCTGGAGATGCCTAGCA |
| | | GCCTGATGCTGGAGGTGCCCGCCCTGGCCGACTTCAACAGAGCCTGGACCGAGCTGACCGACTGG |
| | | CTGAGCCTGCTGGACCAAGTGATCAAGTCTCAGAGAGTGATGGTGGGCGATCTGGAGGACATCAAT |
| | | GAGATGATCATCAAGCAGAAGGCCACCATGCAAGACCTGGAGCAGAGAAGACCTCAGCTGGAGGA |
| | | GCTGATCACCGCCGCTCAGAACCTGAAAAACAAGACGAGCAACCAAGAGGCTAGAACCATCATCA |
| | | CCGACAGAATCGAGAGAATTCAGAATCAGTGGGACGAGGTGCAAGAGCACCTGCAGAACAGAAG |
| | | ACAGCAACTAAACGAAATGCTCAAGGACAGCACACAGTGGCTGGAGGCCAAGGAGGAGGCGGAG |
| | | CAAGTGTTGGGCCAAGCTAGAGCCAAGCTGGAGAGCTGGAAGGAGGGACCCTACACCGTGGACG |
| | | CCATTCAGAAGAAGATCACCGAAACCAAGCAGCTGGCCAAGGACCTGCGACAGTGGCAGACCAA |
| | | CGTGGACGTGGCCAACGATCTTGCCCTGAAGCTGCTGCGCGACTATAGCGCCGACGACACAAGAA |
| | | AGGTGCACATGATCACCGAGAACATCAACGCTAGCTGGCGTAGCATCCACAAGAGAGTGAGCGAG |
| | | AGAGAGGCGGCTTTGGAGGAAACCCATAGACTGCTGCAGCAGTTTCCCCTGGACCTGGAGAAGTT |
| | | CCTGGCCTGGCTGACCGAAGCCGAAACAACCGCCAACGTGCTGCAAGACGCCACAAGAAAGGAG |
| | | AGACTGCTGGAGGACAGCAAGGGCGTGAAGGAGCTGATGAAGCAGTGGCAAGACCTGCAAGGCG |
| | | AGATCGAAGCGCACACCGACGTGTACCACAACCTGGACGAGAACTCTCAGAAGATCCTGAGAAGC |
| | | CTGGAGGGCAGCGACGACGCCGTGCTGTTACAGAGAAGGCTGGACAACATGAACTTCAAGTGGAG |
| | | CGAGCTGAGAAAGAAGAGCCTGAACATCAGAAGCCACCTGGAGGCTAGCAGCGATCAGTGGAAG |
| | | AGACTGCACCTGAGCCTGCAAGAGCTGCTGGTCTGGCTTCAGTTGAAGGACGATGAGCTGAGCAG |
| | | ACAAGCCCCCATCGGCGGCGACTTCCCCGCCGTGCAGAAGCAGAACGACGTGCATAGAGCCTTCA |
| | | AGAGAGAGCTGAAGACCAAGGAGCCCGTGATCATGAGCACCCTGGAAACGGTGAGAATCTTCCTG |
| | | ACCGAGCAGCCCCTGGAGGGCCTGGAGAAGCTGTACCAAGAGCCTAGAGAGCTGCCCCCCGAGG |
| | | AGAGAGCTCAGAACGTGACAAGACTGCTGAGAAAGCAAGCCGAAGAGGTGAACACCGAGTGGGA |
| | | GAAGCTGAACCTGCACAGCGCCGACTGGCAGAGAAAGATCGACGAAACCCTGGAGAGACTGAGA |
| | | GAATTGCAAGAGGCGACCGACGAGCTGGACCTGAAGCTGAGACAAGCCGAGGTCATCAAGGGCA |
| | | GCTGGCAGCCCGTGGGCGATCTATTAATTGACTCTCTACAAGACCACCTGGAGAAAGTGAAGGCCC |
| | | TGAGAGGCGAGATCGCCCCCCTGAAGGAGAACGTGAGCCACGTGAATGATCTGGCACGTCAGCTG |
| | | ACCACCCTGGGCATACAACTGTCCCCCTATAACCTGAGCACACTGGAGGATTTGAATACTAGATGGA |
| | | AGCTGCTGCAAGTGGCCGTGGAGGACAGAGTAAGACAATTACATGAGGCCCACAGAGACTTCGGC |
| | | CCCGCTTCTCAGCACTTCCTGAGCACAAGCGTGCAAGGCCCCTGGGAGAGAGCCATTTCCCCCAAC |
| | | AAGGTCCCCTACTACATCAACCACGAGACACAGACCACCTGCTGGGACCACCCCAAGATGACCGA |
| | | GCTGTATCAGAGCCTCGCCGATCTGAACAATGTGAGATTCAGCGCCTACAGAACCGCCATGAAGCT |
| | | GAGAAGACTGCAGAAGGCCCTGTGCCTGGACTTATTAAGCCTGAGCGCCGCCTGCGACGCCCTGG |
| | | ATCAGCACAACCTGAAGCAAAACGACCAACCCATGGACATCCTGCAGATCATCAACTGCCTCACCA |
| | | CCATATACGACAGACTGGAACAAGAGCACAACAACCTGGTGAACGTGCCCCTGTGCGTGGACATG |
| | | TGCCTGAACTGGCTGCTGAACGTGTACGACACCGGCAGAACCGGCAGAATCAGAGTGCTGAGCTT |
| | | CAAGACCGGCATCATCAGCCTGTGCAAGGCCCACCTGGAGGACAAATACAGATACCTTTTCAAACA |
| | | AGTGGCTAGCAGCACCGGCTTCTGCGACCAAAGAAGACTGGGTCTGCTGCTGCACGACAGCATTC |
| | | AGATCCCTAGACAGCTGGGCGAGGTGGCTAGCTTCGGCGGCAGCAACATCGAGCCTAGCGTGAGA |
| | | AGCTGCTTTCAGTTCGCCAACAACAAGCCCGAGATAGAGGCAGCCCTCTTCTTAGACTGGATGAGA |
| | | CTGGAGCCTCAGAGCATGGTATGGCTGCCGGTCCTGCACAGAGTGGCCGCCGCCGAAACCGCCAA |
| | | GCACCAAGCCAAGTGCAACATCTGCAAGGAGTGCCCCATCATCGGCTTCAGATACAGAAGCCTGAA |
| | | GCACTTCAACTACGACATCTGTCAGAGCTGCTTCTTCAGCGGCAGAGTGGCCAAGGGCCACAAGAT |
| | | GCACTACCCCATGGTGGAGTACTGCACCCCCACCACAAGCGGCGAGGACGTGAGAGACTTCGCCA |
| | | AGGTGTTGAAGAATAAGTTTAGAACCAAGAGATACTTCGCCAAGCACCCTAGAATGGGCTACCTGC |
| | | CCGTGCAGACCGTGCTGGAGGGCGACAACATGGAAACCCCCGTGACCCTGATCAACTTCTGGCCC |
| | | GTGGACAGCGCCCCCGCTAGCAGCCCTCAGCTGAGCCACGACGACACCCACAGCAGAATCGAGCA |
| | | CTACGCTAGCAGACTGGCCGAGATGGAGAACAGCAACGGCAGCTACCTGAACGACAGCATCAGCC |
| | | CGAATGAGAGCATCGACGACGAGCACCTGCTGATTCAGCACTACTGTCAGAGCCTGAACCAAGAC |
| | | AGCCCCCTGTCTCAGCCTAGAAGCCCCGCTCAGATCCTGATCAGCCTGGAGAGCGAGGAGAGAGG |
| | | CGAGCTGGAGAGAATCCTAGCAGATCTGGAGGAAGAGAACAGAAACCTGCAAGCCGAGTATGACC |
| | | GACTGAAACAGCAACATGAGCACAAAGGCCTGAGCCCACTGCCCTCCCCCCCCGAGATGATGCCC |
| | | ACAAGCCCTCAGAGCCCTAGAGACGCCGAGCTGATCGCTGAAGCGAAGTTACTTAGACAACACAA |
| | | GGGCAGACTGGAGGCTAGAATGCAGATCCTGGAGGACCACAACAAGCAGCTGGAGTCTCAGCTGC |
| | | ACAGACTGAGGCAGCTGCTGGAACAGCCCCAAGCCGAGGCCAAAGTGAACGGCACCACCGTGAG |
| | | CAGTCCTAGCACAAGCCTGCAGAGAAGCGACAGCTCTCAGCCCATGCTGCTGAGAGTGGTGGGCT |
| | | CTCAGACAAGCGACAGCATGGGCGAGGAGGATCTGCTGAGTCCGCCCCAAGACACAAGCACCGGC |
| | | CTGGAAGAGGTGATGGAGCAACTGAACAATAGCTTCCCTAGCAGCAGAGGCAGAAACACCCCCGG |
| | | CAAGCCCATGAGAGAGGACACCATGTGATGACTCGAGAATCAACCTCTGGATTACAAAATTTGTGA |
| | | AAGATTGACTGGTATTCTTAACTATGTTGCTCCTTTTACGCTATGTGGATACGCTGCTTTAATGCCTTT |
| | | GTATCATGCTATTGCTTCCCGTATGGCTTTCATTTTCTCCTCCTTGTATAAATCCTGGTTGCTGTCTCTT |
| | | TATGAGGAGTTGTGGCCCGTTGTCAGGCAACGTGGCGTGGTGTGCACTGTGTTTGCTGACGCAACC |
| | | CCCACTGGTTGGGGCATTGCCACCACCTGTCAGCTCCTTTCCGGGACTTTCGCTTTCCCCCTCCCTA |
| | | TTGCCACGGCGGAACTCATCGCCGCCTGCCTTGCCCGCTGCTGGACAGGGGCTCGGCTGTTGGGCA |
| | | CTGACAATTCCGTGGGGTGTGCCTTCTAGTTGCCAGCCATCTGTTGTTTGCCCCTCCCCCGTGCCTT |
| | | CCTTGACCCTGGAAGGTGCCACTCCCACTGTCCTTTCCTAATAAAATGAGGAAATTGCATCGCATTG |
| | | TCTGAGTAGGTGTCATTCTATTCTGGGGGGTGGGGTGGGGCAGGACAGCAAGGGGGAGGATTGGG |
| | | AAGACAATAGCAGGCATGCTGGGGATGCGGTGGGCTCTATGGGAAGATGTCTACTGAGCTGTGCGA |
| | | TCCCTGCTGGGGACTTTCCGCTGGGGACTTTCCGCTGGGGACTTTCCGCCTTCAGCTAAGGAAGCT |
| | | ACCAATATTTAGAGGTACATTTTGTTCTAGAACAAAATGTACCGGTACATTTTGTTCTGGTACATTTT |
| | | GTTCT |
| 27. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 4-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTT |
| | | TGGTGGGAAGAAGTCGAGGACTGCTACGAGCGCGAGGACGTGCAGAAGAAAACCTTCACCAAAT |
| | | GGGTCAACGCCCAGTTCAGCAAGTTCGGCAAGCAGCACATCGAGAACCTGTTCAGCGACCTGCAG |
| | | GATGGCAGAAGGCTGCTGGATCTGCTGGAAGGCCTGACAGGACAGAAGCTGCCCAAAGAGAAGG |
| | | GCAGCACAAGAGTGCACGCCCTGAACAACGTGAACAAGGCCCTGAGAGTGCTGCAGAACAACAA |
| | | CGTGGACCTGGTCAACATCGGCAGCACCGACATCGTGGACGGCAACCACAAACTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATTCTGCTGAGCTGGGTCCGACAGAGCACCCGGAATTACCCTCAA |
| | | GTGAACGTGATCAACTTCACCACCTCTTGGAGCGACGGACTGGCCCTGAATGCCCTGATCCACAGC |
| | | CACAGACCTGACCTGTTCGACTGGAACAGCGTCGTGTGTCAGCAGAGCGCCACACAGAGGCTGGA |
| | | ACACGCCTTCAATATCGCCAGATACCAGCTGGGCATCGAGAAACTGCTGGACCCCGAGGATGTGGA |
| | | CACCACCTATCCTGACAAGAAATCCATCCTCATGTACATCACCAGCCTGTTCCAGGTGCTGCCCCAG |
| | | CAGGTTTCCATCGAGGCCATTCAAGAGGTCGAGATGCTGCCCAGACCTCCTAAAGTGACCAAAGAG |
| | | GAACACTTCCAGCTGCACCACCAGATGCACTACTCTCAGCAGATCACCGTGTCTCTGGCCCAGGGC |
| | | TACGAGAGAACAAGCAGCCCCAAGCCTCGGTTCAAGAGCTACGCCTATACACAGGCCGCCTACGTG |
| | | ACCACCAGCGATCCTACAAGAAGCCCATTTCCTAGCCAGCATCTGGAAGCCCCTGAGGACAAGAGC |
| | | TTTGGCAGCAGCCTGATGGAAAGCGAAGTGAACCTGGACCGCTACCAGACAGCCCTGGAAGAGGT |
| | | TCTGAGCTGGCTGCTGTCTGCCGAGGATACACTGCAGGCTCAGGGCGAGATCAGCAACGACGTGG |
| | | AAGTGGTCAAGGACCAGTTTCACACCCACGAGGGCTACATGATGGACCTGACAGCCCACCAGGGC |
| | | AGAGTGGGCAATATTCTGCAGCTGGGCTCCAAGCTGATCGGCACAGGCAAGCTGAGCGAGGACGA |
| | | AGAGACAGAGGTGCAAGAGCAGATGAACCTGCTGAACAGCAGATGGGAGTGTCTGAGAGTGGCC |
| | | AGCATGGAAAAGCAGAGCAACCTGCACCGGGTGCTGATGGATCTCCAGAACCAGAAGCTGAAAGA |
| | | GCTGAACGACTGGCTGACCAAGACCGAGGAACGGACCCGGAAGATGGAAGAGGAACCTCTGGGA |
| | | CCCGACCTGGAAGATCTGAAAAGACAGGTGCAGCAGCATAAGGTGCTGCAAGAGGACCTCGAACA |
| | | AGAGCAAGTGCGCGTGAACAGCCTGACACACATGGTGGTGGTCGTGGATGAGAGCAGCGGAGATC |
| | | ATGCCACAGCCGCTCTGGAAGAACAGCTGAAGGTGCTGGGAGACAGATGGGCCAATATCTGCCGG |
| | | TGGACCGAGGATAGATGGGTGCTGCTCCAGGACATCCTGCTGAAGTGGCAGCGGCTGACAGAGGA |
| | | ACAGTGCCTGTTTAGCGCCTGGCTGTCCGAGAAAGAGGACGCCGTCAACAAGATCCACACCACCG |
| | | GCTTCAAGGATCAGAATGAGATGCTGAGCAGCCTGCAGAAACTGGCCGTGCTGAAGGCTGACCTG |
| | | GAAAAGAAAAAGCAGTCCATGGGCAAGCTGTACTCCCTGAAGCAGGACCTGCTGAGCACACTGAA |
| | | GAACAAGTCTGTGACCCAGAAAACCGAGGCCTGGCTGGACAACTTCGCCCGGTGTTGGGATAACC |
| | | TGGTGCAGAAGCTGGAAAAGTCCACCGCTCAGATCTCTCAGGCCGTGACCACAACACAGCCTTCT |
| | | CTGACCCAGACCACCGTGATGGAAACAGTGACCACAGTGACAACCCGCGAGCAGATCCTGGTCAA |
| | | GCACGCCCAAGAAGAACTGCCTCCTCCACCTCCTCAGAAGAAACGGCAGATCACAGTGGACAGCG |
| | | AGATCCGGAAGAGACTGGACGTGGACATCACCGAGCTGCACAGCTGGATCACCAGATCCGAAGCC |
| | | GTGCTGCAGTCCCCTGAGTTCGCCATCTTCAGAAAAGAGGGCAACTTCTCCGACCTGAAAGAGAA |
| | | AGTCAACGCCATCGAGAGAGAGAAGGCCGAGAAGTTCCGGAAGCTGCAGGACGCCTCTAGATCTG |
| | | CCCAGGCTCTGGTGGAACAGATGGTCAACGAAGGCGTGAACGCCGACAGCATCAAGCAGGCCTCA |
| | | GAGCAGCTGAACTCCCGGTGGATCGAGTTCTGTCAGCTCCTGAGCGAGAGACTGAACTGGCTGGA |
| | | ATACCAGAACAATATCATTGCCTTCTACAACCAGCTCCAGCAGCTCGAACAGATGACCACCACAGC |
| | | CGAGAATTGGCTGAAGATCCAGCCTACCACACCTAGCGAGCCCACCGCCATTAAGAGCCAGCTGAA |
| | | AATCTGCAAGGACGAAGTGAATCGGCTGAGCGATCTGCAGCCCCAGATCGAAAGACTGAAAATCC |
| | | AGTCTATCGCCCTCAAAGAGAAAGGACAGGGCCCCATGTTCCTGGACGCCGATTTTGTGGCCTTTA |
| | | CCAACCACTTCAAACAGGTGTTCTCCGACGTGCAGGCCCGGGAAAAAGAGCTGCAGACCATCTTC |
| | | GACACCCTGCCTCCAATGAGATACCAAGAGACAATGAGCGCCATCCGGACCTGGGTGCAGCAAAG |
| | | CGAGACAAAGCTGAGCATCCCACAGCTGAGCGTGACCGACTACGAGATCATGGAACAGAGACTGG |
| | | GCGAACTGCAGGCCCTCCAGTCTAGCCTGCAAGAACAGCAGTCCGGCCTGTACTACCTGAGCACA |
| | | ACCGTGAAAGAGATGAGCAAGAAGGCCCCTAGCGAGATCTCCCGGAAGTACCAGAGCGAGTTCGA |
| | | AGAGATCGAAGGCCGGTGGAAGAAGCTGTCTAGCCAGCTGGTTGAGCACTGCCAGAAACTCGAAG |
| | | AACAAATGAACAAGCTGCGCAAGATCCAGAATCACATCCAGACGCTGAAGAAATGGATGGCCGAG |
| | | GTGGACGTGTTCCTGAAAGAAGAGTGGCCCGCTCTGGGCGACTCCGAGATTCTGAAGAAACAGCT |
| | | CAAACAGTGCCGGCTGCTGGTGTCCGATATCCAGACAATCCAGCCAAGCCTGAACTCCGTGAATGA |
| | | AGGCGGCCAGAAGATCAAGAACGAGGCCGAGCCTGAGTTTGCCAGCAGACTGGAAACCGAACTG |
| | | AAAGAACTCAACACCCAGTGGGACCACATGTGCCAGCAAGTGTACGCCCGGAAAGAGGCCCTGAA |
| | | AGGCGGACTCGAAAAGACTGTGTCTCTGCAGAAAGACCTGTCTGAGATGCACGAGTGGATGACCC |
| | | AGGCCGAAGAGGAATACCTGGAACGGGACTTCGAGTACAAGACCCCTGATGAGCTGCAAAAAGCC |
| | | GTCGAGGAAATGAAGCGGGCCAAAGAAGAGGCCCAGCAGAAAGAAGCCAAAGTCAAGCTGCTGA |
| | | CCGAGTCCGTGAATAGCGTTATCGCTCAGGCCCCTCCTGTGGCTCAAGAGGCTCTCAAGAAAGAAC |
| | | TGGAAACTCTGACCACCAACTACCAGTGGCTGTGCACCAGACTGAACGGCAAGTGCAAGACACTG |
| | | GAAGAAGTGTGGGCCTGCTGGCACGAACTGCTGTCCTATCTGGAAAAGGCCAACAAGTGGCTGAA |
| | | CGAGGTGGAATTCAAGCTGAAAACCACCGAGAACATCCCTGGCGGCGCTGAAGAGATTAGCGAGG |
| | | TGCTGGACAGCCTGGAAAACCTGATGAGACACAGCGAGGACAACCCCAATCAGATCAGAATCCTG |
| | | GCTCAGACCCTGACCGATGGCGGCGTGATGGACGAGCTGATCAACGAGGAACTCGAAACCTTCAA |
| | | CAGCCGGTGGCGGGAACTGCACGAGGAAGCTGTTCGGAGGCAGAAGTTGCTGGAACAGTCTATCC |
| | | AGAGCGCACAAGAAACCGAGAAGTCCCTGCACCTGATCCAAGAGAGCCTGACCTTCATCGACAAG |
| | | CAGCTGGCCGCTTATATCGCCGACAAGGTGGACGCTGCTCAGATGCCCCAAGAGGCACAGAAGATT |
| | | CAGAGCGACCTGACCAGCCACGAGATTAGCCTGGAAGAAATGAAGAAGCACAACCAGGGCAAAG |
| | | AGGCCGCTCAGAGAGTCCTGAGCCAGATCGATGTGGCACAGAAAAAGCTCCAGGATGTGTCCATG |
| | | AAGTTTCGGCTGTTTCAGAAGCCCGCCAACTTCGAGCAGAGACTGCAAGAGTCCAAGATGATCCT |
| | | GGACGAAGTCAAAATGCATCTGCCCGCACTGGAAACAAAGTCCGTGGAACAAGAAGTGGTGCAGT |
| | | CCCAGCTGAACCACTGCGTGAACCTGTACAAGAGCCTGTCCGAAGTGAAGTCTGAGGTGGAAATG |
| | | GTCATCAAGACCGGCAGGCAGATCGTCCAGAAGAAGCAGACCGAGAATCCCAAAGAACTCGACG |
| | | AGAGAGTGACCGCTCTGAAGCTGCACTACAATGAGCTGGGCGCCAAAGTGACCGAGCGGAAGCA |
| | | ACAGCTTGAGAAGTGCCTGAAACTGTCTCGGAAGATGCGGAAAGAAATGAACGTGCTGACAGAGT |
| | | GGCTGGCCGCCACCGATATGGAACTGACTAAGAGAAGCGCCGTGGAAGGCATGCCCAGCAACCTG |
| | | GATAGTGAAGTGGCCTGGGGCAAAGCCACTCAGAAAGAGATTGAGAAGCAGAAGGTCCACCTGA |
| | | AGTCCATCACCGAAGTGGGCGAAGCCCTGAAAACCGTGCTGGGAAAGAAAGAGACACTGGTCGA |
| | | GGACAAGCTGTCCCTGCTGAATTCCAACTGGATCGCCGTGACCTCCAGAGCTGAGGAATGGCTGAA |
| | | TCTGCTGCTTGAGTACCAGAAACACATGGAAACGTTCGACCAGAACGTCGACCACATCACAAAGT |
| | | GGATCATCCAGGCTGATACCCTGCTGGACGAGTCCGAGAAGAAGAAACCCCAACAAAAAGAAGAT |
| | | GTGCTGAAGCGGCTGAAAGCCGAGCTGAATGACATCCGGCCTAAGGTGGACAGCACCAGAGATCA |
| | | GGCAGCCAACCTGATGGCCAACAGAGGCGACCACTGTCGGAAGCTCGTGGAACCTCAGATCAGCG |
| | | AGCTTAACCACAGATTTGCCGCCATCAGCCACCGGATCAAGACAGGCAAGGCCAGCATTCCTCTCA |
| | | AAGAGCTTGAGCAGTTCAACAGCGACATCCAAAAGCTGCTCGAACCCCTGGAAGCCGAGATCCAA |
| | | CAGGGCGTCAACCTCAAAGAAGAAGATTTCAACAAGGACATGAACGAGGACAATGAGGGCACCG |
| | | TCAAAGAACTGCTGCAGCGGGGCGATAATCTGCAGCAGCGGATTACCGATGAGCGCAAGCGGGAA |
| | | GAGATCAAGATTAAGCAGCAGCTGCTGCAGACCAAGCACAACGCCCTGAAGGACCTGCGGAGCCA |
| | | GAGAAGAAAGAAGGCCCTGGAAATCAGCCACCAGTGGTATCAGTACAAGCGGCAGGCCGACGAC |
| | | CTGCTGAAGTGCCTGGATGACATCGAGAAGAAGCTGGCCTCTCTGCCCGAGCCTAGGGACGAGAG |
| | | AAAGATCAAAGAGATCGACCGCGAGCTGCAGAAAAAGAAAGAGGAACTGAACGCCGTCCGCAGA |
| | | CAGGCCGAAGGACTTTCTGAAGATGGCGCCGCTATGGCCGTGGAACCCACACAGATTCAGCTGAG |
| | | CAAGCGGTGGCGGGAAATCGAGAGCAAGTTCGCCCAGTTCCGGCGGCTGAATTTCGCCCAGATCC |
| | | ACACCGTGCGGGAAGAGACAATGATGGTCATGACAGAGGACATGCCCCTTGAGATCAGCTACGTGC |
| | | CCAGCACCTACCTGACCGAGATCACCCATGTGTCTCAGGCCCTGCTGGAAGTGGAACAGCTGCTGA |
| | | ATGCCCCTGACCTGTGCGCCAAGGACTTCGAGGACCTGTTCAAGCAAGAGGAAAGCCTGAAGAAC |
| | | ATCAAGGACAGCCTGCAGCAGAGCAGCGGCCGGATCGATATCATCCACAGCAAGAAAACCGCCGC |
| | | ACTGCAGAGCGCCACACCTGTGGAAAGAGTGAAGCTGCAAGAGGCCCTGAGCCAGCTGGATTTCC |
| | | AGTGGGAGAAAGTGAACAAGATGTACAAGGACCGGCAGGGCAGATTCGACCGCAGCGTTGAAAA |
| | | GTGGCGGCGGTTCCACTACGACATCAAGATCTTCAACCAGTGGCTGACCGAGGCCGAGCAGTTCCT |
| | | GAGAAAGACACAGATCCCCGAGAACTGGGAGCACGCCAAGTACAAGTGGTATCTGAAAGAGCTGC |
| | | AGGACGGCATCGGCCAGAGACAGACAGTCGTGCGGACACTGAATGCCACCGGCGAGGAAATCATC |
| | | CAGCAGTCCAGCAAGACCGACGCCAGCATCCTGCAAGAGAAGCTGGGCAGCCTGAACCTGCGGTG |
| | | GCAAGAAGTGTGCAAGCAGCTGAGCGACCGGAAGAAGCGGCTGGAAGAACAGAAGAATATCCTG |
| | | AGCGAGTTCCAGCGGGACCTGAACGAGTTCGTGCTGTGGCTCGAAGAGGCCGACAATATCGCCAG |
| | | CATTCCCCTGGAACCTGGCAAAGAGCAGCAGCTGAAAGAAAAGCTGGAACAAGTGAAACTGCTG |
| | | GTCGAGGAACTGCCCCTGAGACAGGGAATCCTGAAACAGCTGAACGAGACAGGCGGCCCTGTGCT |
| | | GGTGTCTGCCCCTATTTCTCCCGAGGAACAGGACAAGCTGGAAAACAAGCTGAAGCAGACCAACC |
| | | TGCAGTGGATCAAGGTGTCCAGAGCACTGCCTGAGAAGCAGGGCGAGATCGAGGCCCAGATCAAG |
| | | GACCTGGGACAGCTCGAAAAGAAGCTTGAGGACCTCGAAGAACAGCTCAACCATCTGCTGCTTTG |
| | | GCTGAGCCCCATCCGGAACCAGCTGGAAATCTACAACCAGCCTAATCAAGAGGGCCCCTTCGACGT |
| | | GAAAGAAACCGAGATTGCCGTGCAGGCCAAGCAGCCCGATGTGGAAGAGATCCTGTCTAAGGGCC |
| | | AGCACCTGTACAAAGAGAAGCCCGCCACACAGCCCGTGAAGCGGAAACTGGAAGATCTGAGCAG |
| | | CGAGTGGAAGGCCGTGAACCGGCTGCTGCAAGAACTGAGAGCCAAACAGCCTGATCTGGCCCCTG |
| | | GCCTGACAACAATTGGCGCCTCTCCTACACAGACCGTGACACTGGTCACACAGCCTGTGGTCACCA |
| | | AAGAGACAGCCATCAGCAAACTGGAAATGCCCAGCAGTCTGATGCTCGAAGTGCCCGCACTGGCC |
| | | GACTTCAATAGAGCCTGGACCGAGCTGACCGACTGGCTCAGTCTGCTGGACCAAGTCATCAAGTCC |
| | | CAGAGAGTGATGGTCGGAGATCTTGAGGACATCAACGAGATGATCATTAAGCAGAAAGCCACCATG |
| | | CAGGACCTTGAGCAGAGAAGGCCCCAGCTTGAAGAACTGATCACCGCCGCTCAGAACCTGAAAAA |
| | | CAAGACCAGCAATCAAGAAGCCCGGACCATCATCACCGACCGGATCGAGAGAATCCAGAACCAGT |
| | | GGGACGAAGTGCAAGAGCATCTGCAGAACAGACGGCAGCAGCTCAATGAGATGCTGAAGGACAG |
| | | CACACAGTGGCTGGAAGCCAAAGAAGAAGCCGAGCAGGTTCTCGGACAGGCCAGAGCTAAGCTG |
| | | GAATCTTGGAAAGAGGGACCCTACACCGTCGACGCCATCCAGAAGAAGATCACCGAGACAAAGCA |
| | | GCTGGCCAAGGATCTGAGACAGTGGCAGACCAATGTGGACGTGGCCAACGACCTGGCTCTGAAGC |
| | | TGCTGAGAGACTACAGCGCCGACGACACCAGAAAGGTGCACATGATCACCGAAAACATCAACGCC |
| | | TCTTGGCGGAGCATCCACAAGCGGGTGTCAGAGAGAGAAGCCGCTCTGGAAGAAACACATAGACT |
| | | GCTCCAGCAGTTCCCACTCGACCTGGAAAAGTTCCTGGCCTGGCTGACAGAAGCCGAAACCACCG |
| | | CTAACGTGCTCCAGGATGCCACCAGAAAAGAGCGGCTGTTGGAGGACAGCAAGGGCGTCAAAGA |
| | | ACTCATGAAGCAGTGGCAGGATCTGCAAGGGGAGATTGAAGCCCACACCGACGTGTACCACAACC |
| | | TGGACGAGAACAGCCAGAAGATCCTGCGGTCCCTGGAAGGCTCTGATGATGCTGTGCTGCTTCAGA |
| | | GGCGGCTGGACAACATGAACTTCAAGTGGAGCGAGCTGCGGAAGAAGTCCCTGAACATCAGAAGC |
| | | CACCTGGAAGCCTCCAGCGACCAGTGGAAAAGACTGCACCTGTCTCTCCAAGAGCTGCTCGTGTG |
| | | GCTGCAGCTGAAGGATGACGAGCTGAGTAGACAGGCCCCTATCGGCGGAGATTTTCCCGCCGTGCA |
| | | GAAACAGAACGACGTGCACCGGGCCTTCAAGAGAGAGCTGAAAACAAAAGAACCCGTCATCATG |
| | | AGCACCCTGGAAACCGTGCGGATCTTTCTGACCGAGCAGCCTCTGGAAGGACTGGAAAAGCTGTA |
| | | CCAAGAGCCTAGAGAGCTGCCTCCTGAAGAAAGGGCCCAGAACGTGACACGGCTGCTTAGAAAGC |
| | | AGGCCGAGGAAGTGAACACCGAATGGGAGAAGCTGAACCTCCACTCCGCCGACTGGCAGCGGAA |
| | | GATCGATGAGACACTGGAACGGCTGCGGGAACTGCAAGAAGCCACAGACGAGCTGGACCTGAAA |
| | | CTGCGGCAGGCTGAAGTGATCAAAGGCTCCTGGCAGCCAGTGGGCGATCTGCTGATCGATAGCCTG |
| | | CAGGACCATCTGGAAAAAGTCAAGGCCCTGCGGGGAGAGATCGCCCCTCTCAAAGAAAACGTGTC |
| | | CCACGTGAACGATCTGGCCAGGCAGCTGACAACACTGGGCATCCAGCTGAGCCCTTACAACCTGTC |
| | | TACCTTGGAGGACCTGAATACCCGGTGGAAGCTGCTCCAAGTGGCCGTCGAGGATAGAGTGCGGC |
| | | AGCTGCATGAGGCTCACAGAGATTTTGGACCCGCCAGCCAGCACTTCCTGAGCACATCTGTTCAAG |
| | | GCCCCTGGGAGAGAGCTATCAGCCCTAACAAGGTGCCCTACTACATCAACCACGAGACACAGACC |
| | | ACCTGTTGGGATCACCCCAAGATGACCGAACTGTATCAGTCCCTGGCCGACCTGAACAACGTGCGG |
| | | TTTAGCGCCTACCGGACCGCCATGAAGCTGCGCAGACTGCAGAAAGCTCTGTGCCTGGACCTGCTG |
| | | TCTCTGAGCGCTGCTTGTGATGCCCTGGACCAGCATAACCTCAAGCAGAACGACCAGCCTATGGAC |
| | | ATCCTGCAGATCATCAACTGCCTGACCACCATCTACGACCGGCTCGAACAAGAGCACAACAACCTG |
| | | GTCAACGTGCCCCTGTGCGTGGACATGTGCCTGAATTGGCTGCTGAACGTGTACGACACCGGCAGA |
| | | ACCGGCAGGATCAGAGTGCTGAGCTTCAAGACCGGCATCATCTCCCTGTGCAAGGCCCACCTTGAG |
| | | GATAAGTACCGCTACCTGTTTAAGCAGGTCGCCAGCAGCACCGGCTTTTGCGACCAAAGAAGGCTG |
| | | GGACTGCTGCTCCACGACAGCATTCAGATCCCTAGACAGCTGGGCGAAGTGGCCTCTTTCGGCGGC |
| | | TCTAATATCGAGCCTAGCGTGCGGAGCTGCTTCCAGTTCGCCAACAACAAGCCCGAGATTGAGGCC |
| | | GCACTGTTCCTGGACTGGATGAGACTGGAACCCCAGAGCATGGTCTGGCTGCCTGTGCTGCATAGA |
| | | GTGGCCGCAGCCGAAACAGCCAAGCACCAGGCCAAGTGCAACATCTGCAAAGAGTGCCCCATCAT |
| | | CGGCTTCCGGTACAGATCCCTGAAGCACTTCAACTACGATATCTGCCAGTCCTGCTTCTTCAGCGGC |
| | | AGAGTGGCCAAGGGCCACAAGATGCACTACCCCATGGTGGAATACTGCACCCCTACCACCTCTGGC |
| | | GAGGACGTGCGGGATTTTGCCAAGGTGCTCAAGAACAAGTTCCGGACCAAGCGCTACTTCGCTAA |
| | | GCACCCCAGAATGGGCTACCTGCCAGTGCAGACAGTGCTTGAGGGCGACAACATGGAAACCCCTG |
| | | TGACTCTGATCAACTTCTGGCCCGTGGATAGCGCCCCTGCTAGTTCTCCTCAGCTGTCTCACGACGA |
| | | TACCCACAGCAGAATCGAGCACTACGCCTCCAGACTGGCCGAGATGGAAAACAGCAACGGCAGCT |
| | | ACCTGAATGACAGCATCAGCCCCAACGAGAGCATCGACGACGAACATCTGCTCATTCAGCACTACT |
| | | GCCAGAGCCTGAATCAGGACAGCCCTCTGTCTCAGCCTAGAAGCCCTGCACAGATCCTGATCAGCC |
| | | TGGAAAGCGAGGAACGCGGCGAGCTGGAAAGAATCCTGGCAGACCTGGAAGAGGAAAACCGGAA |
| | | CCTGCAGGCCGAATACGACAGACTCAAGCAGCAGCACGAGCACAAGGGACTGTCTCCACTGCCTT |
| | | CTCCACCTGAAATGATGCCCACCTCTCCACAGAGCCCCAGAGATGCCGAGCTGATTGCCGAAGCCA |
| | | AACTGCTGAGGCAGCACAAAGGCAGACTCGAAGCCAGAATGCAGATTCTCGAAGATCACAACAAG |
| | | CAGCTCGAATCCCAGCTGCACAGACTTAGGCAGCTGCTGGAACAGCCTCAGGCAGAGGCCAAAGT |
| | | GAATGGCACCACCGTGTCTAGCCCTAGCACAAGCCTGCAGAGATCCGACAGCAGCCAGCCAATGCT |
| | | TCTGAGAGTCGTGGGCTCCCAGACCAGCGATTCTATGGGCGAAGAGGACCTGCTCAGCCCTCCTCA |
| | | GGATACAAGCACCGGCCTGGAAGAAGTGATGGAACAACTGAACAACAGCTTCCCCAGCAGCAGAG |
| | | GCAGAAACACCCCTGGCAAGCCCATGCGCGAGGACACCATGTGATAACTCGAGAATCAACCTCTG |
| | | GATTACAAAATTTGTGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCTTTCCGGGACTTT |
| | | CGCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGGGAAGATGT |
| | | CTACTGAGCTGTGCGATCCCTGCTGGGGACTTTCCGCTGGGGACTTTCCGCTGGGGACTTTCCGCCT |
| | | TCAGCTAAGGAAGCTACCAATATTTAGAGGTACATTTTGTTCTAGAACAAAATGTACCGGTACATTTT |
| | | GTTCTGGTACATTTTGTTCT |
| 28. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 5-WPRE3- | TGCCCAAGGACTAAAAAAAGGCCATGGAGCCAGAGGGGCGAGGGCAACAGACCTTTCATGGGCA |
| | DTS | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTG |
| | | TGGTGGGAGGAAGTTGAAGATTGTTATGAGAGAGAAGATGTCCAGAAAAAGACCTTTACCAAATG |
| | | GGTGAACGCACAGTTCTCCAAATTTGGGAAACAGCATATAGAAAATTTGTTCAGCGATCTTCAGGAT |
| | | GGGCGCAGGTTGCTTGACCTTCTGGAAGGACTGACCGGGCAGAAGCTGCCCAAAGAGAAAGGGT |
| | | CTACCAGAGTTCACGCTCTGAACAACGTGAATAAGGCACTGCGAGTCCTGCAAAATAACAACGTCG |
| | | ACCTGGTGAACATAGGCAGCACTGATATTGTTGATGGGAACCATAAACTCACACTGGGTCTGATTTG |
| | | GAATATTATACTGCACTGGCAGGTCAAAAATGTTATGAAAAATATTATGGCCGGGCTTCAACAGACC |
| | | AATTCCGAGAAGATCCTGCTGTCTTGGGTTAGGCAGTCCACGAGGAACTACCCTCAAGTGAACGTT |
| | | ATTAACTTCACAACATCATGGAGTGATGGACTTGCCCTGAACGCCTTGATCCACTCCCATAGGCCAG |
| | | ACCTGTTCGATTGGAATAGCGTAGTCTGTCAGCAGAGTGCTACACAACGCCTGGAACACGCCTTTA |
| | | ACATAGCCAGGTATCAGCTTGGAATTGAGAAACTCCTCGACCCTGAAGACGTAGACACCACTTACC |
| | | CCGACAAAAAATCCATTCTTATGTACATAACTTCCTTGTTTCAGGTTCTCCCCCAGCAGGTGTCCATC |
| | | GAGGCTATACAGGAGGTCGAAATGCTCCCCAGACCCCCTAAGGTCACGAAGGAAGAGCACTTCCA |
| | | ACTGCACCATCAGATGCACTACAGTCAACAGATCACTGTGAGTCTCGCACAGGGGTACGAAAGGA |
| | | CCAGCTCCCCTAAACCCCGCTTCAAGTCCTACGCCTATACTCAGGCAGCTTACGTAACCACAAGTGA |
| | | CCCCACGCGGTCACCCTTCCCAAGCCAGCACCTGGAGGCTCCCGAGGACAAAAGCTTTGGATCATC |
| | | CCTGATGGAATCTGAAGTCAATCTCGATAGGTACCAAACCGCTCTGGAGGAGGTCCTCAGTTGGCT |
| | | CCTTTCAGCTGAAGATACTCTGCAGGCACAGGGTGAGATCAGCAATGACGTGGAGGTAGTGAAAG |
| | | ATCAGTTTCACACTCACGAGGGCTATATGATGGACCTGACAGCCCATCAAGGTCGCGTTGGCAATAT |
| | | CCTGCAGTTGGGGTCCAAACTGATTGGTACCGGTAAGTTGTCCGAGGACGAAGAGACAGAAGTGC |
| | | AGGAGCAGATGAACCTGCTTAATTCTAGATGGGAATGCCTCCGCGTGGCCTCTATGGAGAAGCAGT |
| | | CCAATCTCCATCGGGTTCTCATGGACTTGCAGAATCAGAAGCTGAAGGAACTCAACGACTGGTTGA |
| | | CCAAGACTGAGGAGCGCACCAGAAAGATGGAGGAGGAGCCACTCGGTCCTGATCTTGAAGATCTG |
| | | AAACGACAAGTTCAGCAGCACAAGGTCCTGCAGGAGGATCTCGAACAGGAGCAGGTCCGGGTGA |
| | | ACAGCCTGACCCATATGGTTGTCGTTGTCGACGAGTCAAGTGGGGACCATGCTACGGCTGCGCTTG |
| | | AGGAACAGCTGAAGGTCTTGGGCGATAGATGGGCGAATATCTGCCGCTGGACTGAAGATAGATGGG |
| | | TGCTGCTGCAGGATATTCTTTTGAAGTGGCAGCGGTTGACCGAAGAACAATGCTTGTTTAGCGCCT |
| | | GGCTGAGTGAGAAAGAGGATGCGGTGAATAAGATACACACTACTGGCTTCAAAGACCAGAATGAA |
| | | ATGCTTAGCAGCCTGCAGAAACTTGCGGTTCTTAAGGCCGACTTGGAAAAGAAAAAGCAGTCAAT |
| | | GGGTAAGCTGTACTCACTCAAGCAGGACCTGCTGTCAACTTTGAAGAACAAGAGCGTCACGCAAA |
| | | AGACTGAGGCCTGGCTCGACAATTTTGCTAGGTGCTGGGATAACCTGGTGCAGAAGTTGGAAAAGT |
| | | CCACTGCTCAGATCTCTCAAGCCGTGACTACTACCCAGCCCAGCCTGACACAGACTACCGTCATGG |
| | | AAACCGTTACCACTGTGACGACTAGGGAGCAGATACTCGTTAAGCACGCTCAAGAGGAATTGCCGC |
| | | CACCTCCTCCCCAAAAGAAACGCCAGATTACCGTCGACTCTGAAATACGCAAGAGACTGGATGTGG |
| | | ACATCACGGAACTGCACTCTTGGATTACCAGAAGCGAGGCAGTCCTCCAGAGCCCCGAGTTTGCGA |
| | | TCTTCAGGAAGGAAGGCAACTTTTCAGATCTGAAGGAAAAGGTGAACGCCATCGAGCGCGAGAAA |
| | | GCCGAGAAGTTCCGCAAACTGCAGGATGCGAGCCGGTCTGCTCAGGCCTTGGTGGAGCAAATGGT |
| | | GAATGAAGGCGTAAATGCGGACAGCATCAAGCAGGCCTCCGAGCAGCTCAATTCAAGATGGATCG |
| | | AGTTCTGTCAGCTGCTCAGCGAGCGATTGAACTGGCTGGAGTATCAAAATAACATAATCGCGTTTTA |
| | | CAACCAGCTGCAACAGCTGGAACAGATGACGACTACTGCAGAGAACTGGCTTAAAATCCAGCCTA |
| | | CAACTCCTTCTGAACCCACAGCAATCAAAAGCCAGCTCAAGATCTGCAAGGACGAAGTCAACCGC |
| | | CTCTCCGATTTGCAGCCTCAGATAGAACGACTCAAGATCCAAAGTATCGCACTCAAAGAAAAGGGG |
| | | CAAGGCCCTATGTTTCTCGATGCTGATTTTGTGGCTTTCACCAACCATTTTAAGCAAGTGTTTTCAGA |
| | | TGTGCAAGCCCGGGAAAAGGAACTGCAAACCATTTTTGATACCCTGCCTCCAATGCGGTACCAGGA |
| | | GACTATGTCTGCAATTCGCACCTGGGTGCAACAAAGCGAGACGAAACTTTCTATTCCACAACTGTC |
| | | CGTGACAGACTACGAAATTATGGAACAGCGACTGGGAGAACTCCAAGCCCTTCAGTCCAGCCTTCA |
| | | GGAGCAGCAGTCAGGATTGTACTATCTGTCAACCACCGTGAAGGAAATGTCAAAAAAGGCTCCCTC |
| | | CGAAATATCTCGCAAGTATCAGTCTGAGTTTGAAGAGATTGAAGGGAGATGGAAGAAACTGTCCTC |
| | | ACAGCTGGTTGAGCACTGTCAGAAGTTGGAAGAGCAGATGAACAAGTTGCGCAAGATTCAAAATC |
| | | ACATACAGACACTCAAAAAGTGGATGGCGGAGGTTGACGTCTTCCTTAAAGAGGAGTGGCCAGCC |
| | | CTGGGCGACTCAGAGATCCTGAAAAAGCAACTGAAACAGTGCCGACTGCTGGTGTCCGACATCCA |
| | | GACAATACAGCCAAGTTTGAATAGCGTGAATGAGGGTGGACAAAAAATCAAAAACGAGGCTGAGC |
| | | CCGAGTTCGCCTCCAGATTGGAAACCGAGCTGAAGGAACTTAATACACAGTGGGATCATATGTGTC |
| | | AGCAGGTTTATGCCCGGAAGGAAGCACTGAAAGGGGGGCTGGAGAAAACCGTGAGCCTTCAGAA |
| | | GGACCTTAGCGAGATGCACGAGTGGATGACCCAGGCGGAGGAAGAGTATCTCGAACGGGATTTTG |
| | | AGTATAAGACACCAGATGAACTTCAGAAGGCAGTCGAAGAGATGAAACGGGCAAAGGAAGAGGC |
| | | TCAGCAGAAGGAAGCAAAGGTTAAACTTCTGACAGAATCCGTTAACAGCGTTATAGCCCAAGCTCC |
| | | CCCCGTTGCCCAGGAAGCTCTTAAAAAGGAACTGGAAACACTGACAACAAACTACCAGTGGCTGT |
| | | GCACCCGCTTGAACGGCAAATGCAAAACCCTGGAAGAGGTCTGGGCTTGTTGGCATGAACTCCTG |
| | | TCATACCTGGAAAAAGCTAATAAGTGGCTGAATGAGGTGGAGTTTAAATTGAAAACGACCGAAAAC |
| | | ATCCCTGGCGGTGCAGAGGAGATATCTGAGGTGCTCGATAGCCTGGAGAATCTTATGAGGCATAGCG |
| | | AGGATAACCCCAACCAGATTAGGATACTGGCCCAAACTCTGACCGACGGCGGAGTGATGGACGAG |
| | | CTGATAAACGAGGAACTGGAAACCTTTAATTCACGCTGGCGAGAACTTCACGAGGAGGCAGTCAG |
| | | GCGCCAGAAACTGCTGGAGCAGTCCATCCAGAGTGCCCAGGAGACTGAGAAGTCACTTCATCTGA |
| | | TCCAGGAGAGTTTGACGTTTATCGACAAGCAGCTTGCCGCCTACATTGCAGACAAGGTCGATGCTG |
| | | CTCAGATGCCGCAAGAGGCTCAGAAAATCCAGAGCGACCTGACAAGCCACGAGATTAGCCTGGAG |
| | | GAAATGAAAAAGCACAACCAGGGTAAAGAGGCCGCCCAGCGGGTTCTGAGCCAGATCGATGTCGC |
| | | ACAGAAGAAACTCCAAGATGTAAGCATGAAGTTCCGACTGTTTCAGAAACCAGCGAACTTTGAGC |
| | | AGCGACTGCAGGAGTCTAAAATGATCCTCGATGAGGTGAAAATGCATCTGCCAGCCCTGGAAACTA |
| | | AGAGCGTGGAGCAGGAGGTGGTTCAGAGTCAGCTGAACCACTGCGTAAACCTCTATAAAAGTCTT |
| | | AGTGAAGTGAAGAGCGAGGTGGAGATGGTGATTAAGACTGGTAGACAGATTGTACAGAAAAAGCA |
| | | GACGGAGAACCCCAAAGAGTTGGATGAACGGGTGACAGCCCTTAAGCTCCACTATAACGAGCTGG |
| | | GAGCCAAAGTGACTGAGAGAAAGCAGCAACTTGAAAAATGTCTGAAGCTGAGCAGAAAGATGCG |
| | | GAAGGAAATGAATGTGCTGACAGAGTGGCTGGCTGCGACCGACATGGAACTTACAAAGAGGAGCG |
| | | CCGTGGAAGGCATGCCTTCCAATCTGGATAGTGAGGTCGCTTGGGGCAAAGCTACACAGAAGGAA |
| | | ATTGAGAAGCAAAAAGTGCATCTTAAGAGCATTACTGAAGTTGGCGAGGCTTTGAAAACCGTCCTC |
| | | GGCAAGAAAGAGACTCTGGTAGAAGATAAACTCTCACTGCTGAACTCAAACTGGATCGCAGTCAC |
| | | TAGTCGGGCCGAAGAGTGGCTGAATTTGCTGTTGGAGTACCAAAAGCATATGGAGACTTTCGATCA |
| | | GAATGTCGACCACATTACGAAGTGGATAATCCAGGCGGACACACTGTTGGATGAGTCCGAGAAAAA |
| | | GAAACCTCAGCAGAAGGAAGACGTGCTCAAACGACTCAAAGCCGAACTGAACGACATCAGGCCC |
| | | AAAGTTGATTCCACCCGGGATCAAGCGGCCAATCTCATGGCCAACCGGGGGGATCACTGCAGAAA |
| | | GTTGGTAGAACCTCAGATAAGCGAACTTAATCATAGATTTGCCGCCATATCACATAGGATTAAGACC |
| | | GGTAAGGCTTCAATCCCCCTGAAGGAACTCGAACAGTTTAATAGTGACATCCAGAAGCTCCTGGAG |
| | | CCTCTCGAAGCCGAGATTCAACAGGGTGTAAACCTTAAGGAAGAGGATTTTAATAAGGATATGAAT |
| | | GAGGACAATGAAGGCACGGTCAAAGAGCTCCTCCAGAGGGGAGACAATTTGCAGCAGCGGATCAC |
| | | CGATGAACGCAAACGCGAAGAGATCAAAATTAAGCAGCAGCTCCTCCAAACAAAGCACAATGCCC |
| | | TTAAAGACCTCAGAAGCCAGCGGCGGAAAAAGGCTCTGGAAATCAGCCATCAATGGTATCAGTACA |
| | | AGCGGCAAGCGGACGATCTCCTGAAATGTCTGGATGATATCGAGAAAAAACTGGCCAGTCTGCCAG |
| | | AGCCCAGAGATGAGCGCAAAATCAAGGAAATCGACAGGGAGCTTCAGAAGAAAAAAGAGGAGCT |
| | | GAACGCCGTCCGAAGGCAGGCCGAAGGGCTGTCAGAAGATGGGGCCGCCATGGCAGTGGAACCTA |
| | | CCCAGATTCAACTGTCCAAGCGCTGGAGAGAGATTGAAAGTAAGTTCGCACAATTCCGGAGGCTTA |
| | | ATTTTGCTCAGATTCACACAGTTAGGGAGGAAACCATGATGGTGATGACCGAAGACATGCCACTCG |
| | | AAATCAGCTACGTCCCTAGCACATATCTCACGGAGATTACCCACGTGTCCCAGGCACTGCTGGAAGT |
| | | CGAGCAATTGTTGAACGCCCCTGATCTCTGCGCCAAGGATTTCGAAGACTTGTTCAAGCAAGAGGA |
| | | AAGCCTCAAGAACATCAAAGACAGTCTTCAGCAGTCCTCAGGCAGAATTGATATTATCCACTCCAA |
| | | GAAAACAGCCGCTCTCCAGAGCGCTACCCCTGTCGAACGAGTGAAACTGCAAGAGGCCTTGTCTC |
| | | AGTTGGACTTCCAGTGGGAGAAAGTCAACAAGATGTACAAGGACAGGCAGGGTCGGTTCGACCGG |
| | | AGCGTCGAAAAATGGCGGAGATTCCATTATGACATCAAGATATTCAATCAGTGGCTTACAGAGGCGG |
| | | AGCAATTTTTGAGAAAGACGCAGATTCCCGAAAACTGGGAGCATGCCAAGTACAAGTGGTACCTC |
| | | AAAGAGCTCCAGGATGGAATCGGCCAGAGACAGACCGTAGTTAGAACACTGAACGCAACTGGAGA |
| | | AGAGATTATCCAGCAGTCCTCCAAAACTGACGCCTCTATCCTCCAGGAGAAGCTTGGCAGCCTTAA |
| | | CCTGCGGTGGCAGGAGGTGTGTAAACAACTGTCAGATAGGAAAAAAAGACTTGAGGAACAGAAG |
| | | AACATTCTGAGCGAGTTCCAACGAGATCTCAACGAGTTCGTACTGTGGCTGGAGGAGGCCGACAA |
| | | CATCGCTTCAATCCCCCTGGAACCCGGCAAGGAACAGCAGCTTAAGGAAAAATTGGAACAGGTAA |
| | | AACTGTTGGTAGAGGAGCTCCCATTGCGGCAGGGCATTCTCAAGCAGCTGAATGAAACTGGAGGTC |
| | | CCGTTCTGGTCAGTGCGCCCATAAGCCCGGAAGAGCAGGACAAGTTGGAAAACAAGCTGAAGCAA |
| | | ACAAATCTGCAGTGGATCAAAGTGAGCCGGGCTCTTCCAGAAAAGCAAGGCGAGATTGAAGCCCA |
| | | GATTAAGGATCTTGGCCAGCTTGAGAAAAAGCTGGAGGATCTGGAAGAGCAGCTGAACCATTTGCT |
| | | TCTGTGGCTGAGTCCCATTAGAAACCAGCTGGAAATCTACAACCAGCCAAACCAAGAGGGCCCTTT |
| | | CGACGTCAAGGAAACTGAGATAGCAGTCCAGGCAAAACAGCCCGATGTCGAAGAAATCTTGTCTA |
| | | AGGGGCAGCACCTCTACAAAGAGAAACCTGCCACCCAGCCCGTGAAGAGAAAACTGGAAGACCT |
| | | CTCTTCAGAGTGGAAGGCTGTAAACAGGTTGCTCCAGGAGCTGAGAGCTAAGCAACCTGACCTCG |
| | | CACCCGGCCTGACTACTATTGGCGCCAGCCCGACCCAAACCGTCACACTGGTGACCCAGCCTGTAG |
| | | TCACAAAAGAAACCGCCATCTCCAAACTGGAGATGCCTTCAAGCCTGATGCTGGAGGTGCCTGCTC |
| | | TGGCCGATTTCAACAGAGCCTGGACAGAGCTGACAGACTGGCTGTCTCTGCTCGACCAAGTGATAA |
| | | AGTCACAGCGCGTCATGGTGGGTGACCTGGAGGACATCAATGAGATGATCATAAAGCAAAAGGCTA |
| | | CCATGCAGGACTTGGAGCAGCGCAGGCCGCAGTTGGAAGAACTGATTACAGCAGCCCAGAACCTC |
| | | AAGAACAAGACTTCAAATCAGGAGGCAAGGACCATCATAACAGATAGAATCGAGAGGATTCAGAA |
| | | CCAATGGGATGAGGTGCAGGAGCACCTGCAGAACAGAAGGCAGCAGCTGAACGAAATGCTGAAA |
| | | GACTCCACACAGTGGCTGGAGGCCAAAGAGGAGGCAGAACAGGTGCTGGGCCAGGCTAGGGCCA |
| | | AGCTGGAAAGTTGGAAGGAAGGGCCCTACACCGTGGACGCCATACAGAAGAAAATAACGGAGAC |
| | | AAAACAGCTGGCCAAAGACCTCCGCCAATGGCAGACCAACGTAGACGTCGCCAATGACCTGGCAC |
| | | TCAAGTTGTTGAGAGATTATTCCGCAGATGACACCCGAAAAGTACACATGATCACTGAGAATATCAA |
| | | CGCCAGTTGGCGGAGCATTCACAAACGGGTGAGCGAGAGAGAAGCCGCCCTTGAGGAGACGCATC |
| | | GCCTTTTGCAGCAATTTCCGCTTGACCTGGAAAAGTTTCTGGCTTGGTTGACAGAGGCCGAGACAA |
| | | CTGCTAACGTGCTGCAAGATGCCACCCGCAAAGAGCGGCTTCTTGAGGACTCTAAAGGGGTGAAA |
| | | GAATTGATGAAGCAGTGGCAGGACCTCCAGGGCGAAATCGAGGCCCATACTGATGTCTACCACAAC |
| | | CTGGATGAAAATAGTCAGAAAATCCTGAGATCCCTGGAAGGGAGTGATGATGCGGTGCTGCTGCAG |
| | | CGCCGACTCGATAACATGAACTTTAAGTGGTCTGAACTCCGGAAAAAAAGCCTGAACATTCGGAGC |
| | | CATCTGGAGGCTTCTTCTGATCAGTGGAAGAGACTTCATCTGTCCTTGCAGGAGCTTCTGGTGTGGC |
| | | TGCAGCTGAAAGACGACGAGCTGTCAAGGCAGGCACCAATAGGGGGCGACTTCCCCGCTGTTCAG |
| | | AAACAAAATGATGTGCACAGGGCATTCAAGCGGGAACTGAAGACGAAAGAACCAGTGATTATGAG |
| | | CACTCTGGAGACGGTACGCATCTTTCTGACCGAGCAGCCACTTGAAGGCCTCGAAAAGTTGTACCA |
| | | GGAGCCACGGGAATTGCCTCCCGAAGAACGGGCTCAGAATGTTACCCGACTGCTTCGCAAGCAGG |
| | | CTGAGGAAGTGAACACTGAGTGGGAAAAACTGAATCTGCACAGTGCCGATTGGCAGCGCAAAATT |
| | | GACGAGACATTGGAGCGCTTGCGGGAGCTGCAGGAAGCCACAGATGAACTTGATCTGAAACTCAG |
| | | ACAGGCCGAGGTCATAAAGGGTTCTTGGCAACCCGTAGGCGACCTTTTGATTGACTCACTGCAGGA |
| | | TCATCTGGAGAAAGTAAAGGCGCTGCGGGGGGAAATTGCTCCGCTGAAGGAAAACGTGAGCCATG |
| | | TGAATGATCTGGCTCGGCAATTGACTACCCTGGGTATCCAGCTGTCCCCCTACAATCTGAGCACTCT |
| | | CGAAGACCTCAACACAAGATGGAAACTGCTGCAGGTCGCCGTGGAGGACAGGGTGAGGCAGCTG |
| | | CATGAGGCACACAGGGACTTCGGTCCGGCAAGCCAACACTTCCTGTCTACTTCTGTTCAAGGGCCT |
| | | TGGGAACGCGCTATTTCCCCAAACAAAGTGCCGTACTATATTAACCATGAAACTCAGACAACCTGCT |
| | | GGGATCATCCTAAAATGACTGAGCTGTACCAGAGCCTCGCCGATCTGAATAACGTGAGGTTTAGCG |
| | | CATACAGAACCGCAATGAAGCTCAGGAGACTGCAGAAGGCACTTTGCCTGGACCTCCTGAGCCTCT |
| | | CCGCTGCCTGCGATGCCCTCGACCAGCACAACCTGAAGCAGAATGATCAGCCTATGGACATCCTGC |
| | | AAATCATAAATTGTCTGACGACAATCTACGACCGGCTCGAACAGGAGCACAATAATCTCGTGAACG |
| | | TTCCACTTTGTGTGGATATGTGTCTCAACTGGCTGTTGAACGTCTATGACACCGGGAGAACTGGAA |
| | | GGATCCGCGTTCTGTCCTTTAAGACTGGAATCATCAGCTTGTGTAAGGCGCACCTTGAGGACAAATA |
| | | TCGATACCTGTTTAAGCAGGTTGCAAGCTCCACTGGATTCTGCGACCAGAGGCGCCTCGGGCTGCT |
| | | GCTTCATGATAGTATCCAAATCCCACGCCAACTCGGAGAGGTGGCAAGCTTCGGCGGGAGCAATAT |
| | | TGAGCCAAGCGTCAGATCTTGTTTCCAGTTTGCCAACAATAAGCCTGAGATCGAAGCCGCACTGTT |
| | | TTTGGATTGGATGCGGTTGGAGCCCCAGAGTATGGTGTGGTTGCCCGTGCTGCATAGGGTCGCTGCC |
| | | GCTGAAACCGCCAAACACCAGGCCAAGTGCAATATTTGCAAGGAATGTCCCATCATCGGGTTTAGA |
| | | TACCGGAGCCTGAAGCACTTCAATTATGATATCTGCCAGTCTTGTTTTTTTTCCGGCCGAGTTGCTAA |
| | | GGGACACAAGATGCATTACCCAATGGTGGAATATTGTACGCCAACCACCAGCGGCGAAGATGTCAG |
| | | AGATTTTGCAAAAGTACTCAAGAACAAGTTCCGCACCAAGCGATATTTCGCGAAGCACCCCCGGAT |
| | | GGGCTACCTCCCAGTGCAGACGGTTCTGGAAGGCGATAACATGGAAACTCCAGTCACCCTGATCAA |
| | | TTTCTGGCCTGTGGACTCTGCGCCTGCCTCTTCACCTCAGCTCTCCCACGATGACACCCATTCCAGA |
| | | ATCGAGCACTACGCGTCTCGACTGGCGGAGATGGAGAACAGCAATGGTTCTTACCTCAATGACAGC |
| | | ATTAGCCCCAACGAAAGCATCGATGACGAGCACCTCTTGATTCAACACTACTGCCAGTCCCTGAATC |
| | | AGGACAGTCCTCTGTCCCAACCCAGGAGCCCAGCCCAGATACTGATCTCTCTCGAATCAGAAGAAA |
| | | GAGGGGAGTTGGAGCGAATTTTGGCCGACCTGGAGGAGGAGAATAGAAATCTTCAGGCCGAGTAC |
| | | GATAGACTGAAGCAGCAGCACGAGCACAAGGGCCTTTCTCCACTGCCCTCTCCACCGGAAATGATG |
| | | CCAACCTCCCCTCAAAGTCCCAGGGACGCTGAGCTCATTGCCGAGGCAAAGCTGTTGCGCCAGCA |
| | | CAAAGGACGCCTGGAGGCGAGAATGCAGATCCTGGAAGACCATAACAAGCAGCTTGAGTCTCAGC |
| | | TGCACCGACTTAGACAGCTGTTGGAGCAGCCACAAGCGGAAGCAAAGGTCAATGGAACTACCGTG |
| | | AGTTCCCCCAGTACAAGTCTGCAGCGCTCTGACTCAAGCCAGCCAATGCTGCTCCGGGTAGTCGGT |
| | | TCTCAGACTAGTGATTCAATGGGGGAGGAGGATCTTCTTTCCCCACCCCAAGACACCTCAACAGGG |
| | | CTTGAGGAAGTGATGGAGCAGCTGAACAACTCCTTCCCCTCTTCACGGGGGAGAAATACTCCTGGG |
| | | AAGCCTATGAGAGAGGATACCATGTAATGACTCGAGAATCAACCTCTGGATTACAAAATTTGTGAAA |
| | | GATTGACTGGTATTCTTAACTATGTTGCTCCTTTTACGCTATGTGGATACGCTGCTTTAATGCCTTTGT |
| | | ATCATGCTATTGCTTCCCGTATGGCTTTCATTTTCTCCTCCTTGTATAAATCCTGGTTGCTGTCTCTTTA |
| | | TGAGGAGTTGTGGCCCGTTGTCAGGCAACGTGGCGTGGTGTGCACTGTGTTTGCTGACGCAACCCC |
| | | CACTGGTTGGGGCATTGCCACCACCTGTCAGCTCCTTTCCGGGACTTTCGCTTTCCCCCTCCCTATT |
| | | GCCACGGCGGAACTCATCGCCGCCTGCCTTGCCCGCTGCTGGACAGGGGCTCGGCTGTTGGGCACT |
| | | GACAATTCCGTGGGGTGTGCCTTCTAGTTGCCAGCCATCTGTTGTTTGCCCCTCCCCCGTGCCTTCC |
| | | TTGACCCTGGAAGGTGCCACTCCCACTGTCCTTTCCTAATAAAATGAGGAAATTGCATCGCATTGTC |
| | | TGAGTAGGTGTCATTCTATTCTGGGGGGTGGGGTGGGGCAGGACAGCAAGGGGGAGGATTGGGAA |
| | | GACAATAGCAGGCATGCTGGGGATGCGGTGGGCTCTATGGGAAGATGTCTACTGAGCTGTGCGATC |
| | | CCTGCTGGGGACTTTCCGCTGGGGACTTTCCGCTGGGGACTTTCCGCCTTCAGCTAAGGAAGCTAC |
| | | CAATATTTAGAGGTACATTTTGTTCTAGAACAAAATGTACCGGTACATTTTGTTCTGGTACATTTTGTT |
| | | CT |
| 29. | NP-CBA- | TGCATGCGGCCGCCGTTACATAACTTACGGTAAATGGCCCGCCTGGCTGACCGCCCAACGACCCCC |
| | hBGi- | GCCCATTGACGTCAATAATGACGTATGTTCCCATAGTAACGCCAATAGGGACTTTCCATTGACGTCA |
| | EGFP-P2A- | ATGGGTGGAGTATTTACGGTAAACTGCCCACTTGGCAGTACATCAAGTGTATCATATGCCAAGTACG |
| | Fluc- | CCCCCTATTGACGTCAATGACGGTAAATGGCCCGCCTGGCATTATGCCCAGTACATGACCTTATGGG |
| | WPRE3- | ACTTTCCTACTTGGCAGTACATCTACGTATTAGTCATCGCTATTACCATGGTCGAGGTGAGCCCCACG |
| | DTS | TTCTGCTTCACTCTCCCCATCTCCCCCCCCTCCCCACCCCCAATTTTGTATTTATTTATTTTTTAATTAT |
| | | TTTGTGCAGCGATGGGGGCGGGGGGGGGGGGGGCGCGCGCCAGGCGGGGCGGGGCGGGGCGAGG |
| | | GGCGGGGCGGGGCGAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCGCGGCGGGCGGGAG |
| | | TCTCTGGATCCACTGCTTAAATACGGACGAGGACAGGGCCCTGTCTCCTCAGCTTCAGGCACCACC |
| | | ACTGACCTGGGACAGTGAATCGTAAGTACTAGCAGCTACAATCCAGCTACCATTCTGCTTTTATTTTA |
| | | TGGTTGGGATAAGGCTGGATTATTCTGAGTCCAAGCTAGGCCCTTTTGCTAATCATGTTCATACCTCT |
| | | TATCTTCCTCCCACAGCTCCTGGGCAACGTGCTGGTCTGTGTGCTGGCCCATCACTTTGGCAAAGAA |
| | | TTGCGATCGCCACCATGGTGAGCAAGGGCGAGGAGCTGTTCACCGGGGTGGTGCCCATCCTGGTCG |
| | | AGCTGGACGGCGACGTAAACGGCCACAAGTTCAGCGTGTCCGGCGAGGGCGAGGGCGATGCCACC |
| | | TACGGCAAGCTGACCCTGAAGTTCATCTGCACCACCGGCAAGCTGCCCGTGCCCTGGCCCACCCTC |
| | | GTGACCACCCTGACCTACGGCGTGCAGTGCTTCAGCCGCTACCCCGACCACATGAAGCAGCACGA |
| | | CTTCTTCAAGTCCGCCATGCCCGAAGGCTACGTCCAGGAGCGCACCATCTTCTTCAAGGACGACGG |
| | | CAACTACAAGACCCGCGCCGAGGTGAAGTTCGAGGGCGACACCCTGGTGAACCGCATCGAGCTGA |
| | | AGGGCATCGACTTCAAGGAGGACGGCAACATCCTGGGGCACAAGCTGGAGTACAACTACAACAGC |
| | | CACAACGTCTATATCATGGCCGACAAGCAGAAGAACGGCATCAAGGTGAACTTCAAGATCCGCCAC |
| | | AACATCGAGGACGGCAGCGTGCAGCTCGCCGACCACTACCAGCAGAACACCCCCATCGGCGACGG |
| | | CCCCGTGCTGCTGCCCGACAACCACTACCTGAGCACCCAGTCCGCCCTGAGCAAAGACCCCAACG |
| | | AGAAGCGCGATCACATGGTCCTGCTGGAGTTCGTGACCGCCGCCGGGATCACTCTCGGCATGGACG |
| | | AGCTGTACAAGAATCGCGCTACTAACTTCAGCCTGCTGAAGCAGGCTGGAGACGTGGAGGAGAAC |
| | | CCTGGACCTATGGAAGACGCCAAAAACATAAAGAAAGGCCCGGCGCCATTCTATCCGCTGGAAGAT |
| | | GGAACCGCTGGAGAGCAACTGCATAAGGCTATGAAGAGATACGCCCTGGTTCCTGGAACAATTGCT |
| | | TTTACAGATGCACATATCGAGGTGGACATCACTTACGCTGAGTACTTCGAAATGTCCGTTCGGTTGG |
| | | CAGAAGCTATGAAACGATATGGGCTGAATACAAATCACAGAATCGTCGTATGCAGTGAAAACTCTCT |
| | | TCAATTCTTTATGCCGGTGTTGGGCGCGTTATTTATCGGAGTTGCAGTTGCGCCCGCGAACGACATTT |
| | | ATAATGAACGTGAATTGCTCAACAGTATGGGCATTTCGCAGCCTACCGTGGTGTTCGTTTCCAAAAA |
| | | GGGGTTGCAAAAAATTTTGAACGTGCAAAAAAAGCTCCCAATCATCCAAAAAATTATTATCATGGAT |
| | | TCTAAAACGGATTACCAGGGATTTCAGTCGATGTACACGTTCGTCACATCTCATCTACCTCCCGGTTT |
| | | TAATGAATACGATTTTGTGCCAGAGTCCTTCGATAGGGACAAGACAATTGCACTGATCATGAACTCC |
| | | TCTGGATCTACTGGTCTGCCTAAAGGTGTCGCTCTGCCTCATAGAACTGCCTGCGTGAGATTCTCGC |
| | | ATGCCAGAGATCCTATTTTTGGCAATCAAATCATTCCGGATACTGCGATTTTAAGTGTTGTTCCATTC |
| | | CATCACGGTTTTGGAATGTTTACTACACTCGGATATTTGATATGTGGATTTCGAGTCGTCTTAATGTAT |
| | | AGATTTGAAGAAGAGCTGTTTCTGAGGAGCCTTCAGGATTACAAGATTCAAAGTGCGCTGCTGGTG |
| | | CCAACCCTATTCTCCTTCTTCGCCAAAAGCACTCTGATTGACAAATACGATTTATCTAATTTACACGA |
| | | AATTGCTTCTGGTGGCGCTCCCCTCTCTAAGGAAGTCGGGGAAGCGGTTGCCAAGAGGTTCCATCT |
| | | GCCAGGTATCAGGCAAGGATATGGGCTCACTGAGACTACATCAGCTATTCTGATTACACCCGAGGGG |
| | | GATGATAAACCGGGCGCGGTCGGTAAAGTTGTTCCATTTTTTGAAGCGAAGGTTGTGGATCTGGATA |
| | | CCGGGAAAACGCTGGGCGTTAATCAAAGAGGCGAACTGTGTGTGAGAGGTCCTATGATTATGTCCG |
| | | GTTATGTAAACAATCCGGAAGCGACCAACGCCTTGATTGACAAGGATGGATGGCTACATTCTGGAG |
| | | ACATAGCTTACTGGGACGAAGACGAACACTTCTTCATCGTTGACCGCCTGAAGTCTCTGATTAAGTA |
| | | CAAAGGCTATCAGGTGGCTCCCGCTGAATTGGAATCCATCTTGCTCCAACACCCCAACATCTTCGAC |
| | | GCAGGTGTCGCAGGTCTTCCCGACGATGACGCCGGTGAACTTCCCGCCGCCGTTGTTGTTTTGGAG |
| | | CACGGAAAGACGATGACGGAAAAAGAGATCGTGGATTACGTCGCCAGTCAAGTAACAACCGCGAA |
| | | AAAGTTGCGCGGAGGAGTTGTGTTTGTGGACGAAGTACCGAAAGGTCTTACCGGAAAACTCGACG |
| | | CAAGAAAAATCAGAGAGATCCTCATAAAGGCCAAGAAGGGCGGAAAGATCGCCGTGTAACTCGAG |
| | | AATCAACCTCTGGATTACAAAATTTGTGAAAGATTGACTGGTATTCTTAACTATGTTGCTCCTTTTAC |
| | | GCTATGTGGATACGCTGCTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGGGGACTTTCCGCTG |
| | | GGGACTTTCCGCCTTCAGCTAAGGAAGCTACCAATATTTAGAGGTACATTTTGTTCTAGAACAAAAT |
| | | GTACCGGTACATTTTGTTCTGGTACATTTTGTTCTATTAAGGACAACGTAAGTATAGCGCATAGACAC |
| | | GTGGCGCCATCGGATCCCGGGTGGCTTGTTGTCCACAACCATTAAACCTTAAAAGCTTTAAAAGCCT |
| | | TATATATTCTTTTTTTTCTTATAAAACTTAAAACCTTAGAGGCTATTTAAGTTGCTGATTTATATTAATT |
| | | TTATTGTTCAAACATGAGAGCTTAGTACGTGAAACATGAGAGCTTAGTACATTAGCCATGAGAGCTTA |
| | | GTACATTAGCCATGAGGGTTTAGTTCATTAAACATGAGAGCTTAGTACATTAAACATGAGAGCTTAG |
| | | TACATTAAACATGAGAGCTTAGTACATACTATCAACAGGTTGAACTGCTGATCTGTACAGTAGAATT |
| | | GGTAAAGAGAGTTGTGTAAAATATTGAGTTCGCACATCTTGTTGTCTGATTATTGATTTTTGGCGAAA |
| | | CCATTTGATCATATGACAAGATGTGTATCTACCTTAACTTAATGATTTTGATAAAAATCATTA |
| 30. | NP- | CTAGAAGCTGCATGTCTAAGCTAGACCCTTCAGATTAAAAATAACTGAGGTAAGGGCCTGGGTAGG |
| | MHCK7- | GGAGGTGGTGTGAGACGCTCCTGTCTCTCCTCTATCTGCCCATCGGCCCTTTGGGGAGGAGGAATG |
| | hBGi- | TGCCCAAGGACTAAAAAAAGGCCATGGAGCCAGAGGGGCGAGGGCAACAGACCTTTCATGGGCA |
| | EGFP-P2A- | AACCTTGGGGCCCTGCTGTCTAGCATGCCCCACTACGGGTCTAGGCTGCCCATGTAAGGAGGCAAG |
| | Fluc- | GCCTGGGGACACCCGAGATGCCTGGTTATAATTAACCCAGACATGTGGCTGCCCCCCCCCCCCCAA |
| | WPRE3- | CACCTGCTGCCTCTAAAAATAACCCTGTCCCTGGTGGATCCCCTGCATGCGAAGATCTTCGAACAAG |
| | DTS | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGGTG |
| | | AGCAAGGGCGAGGAGCTGTTCACCGGGGTGGTGCCCATCCTGGTCGAGCTGGACGGCGACGTAAA |
| | | CGGCCACAAGTTCAGCGTGTCCGGCGAGGGCGAGGGCGATGCCACCTACGGCAAGCTGACCCTGA |
| | | AGTTCATCTGCACCACCGGCAAGCTGCCCGTGCCCTGGCCCACCCTCGTGACCACCCTGACCTACG |
| | | GCGTGCAGTGCTTCAGCCGCTACCCCGACCACATGAAGCAGCACGACTTCTTCAAGTCCGCCATGC |
| | | CCGAAGGCTACGTCCAGGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGGGCATCGACTTCAAGGA |
| | | GGACGGCAACATCCTGGGGCACAAGCTGGAGTACAACTACAACAGCCACAACGTCTATATCATGGC |
| | | CGACAAGCAGAAGAACGGCATCAAGGTGAACTTCAAGATCCGCCACAACATCGAGGACGGCAGC |
| | | GTGCAGCTCGCCGACCACTACCAGCAGAACACCCCCATCGGCGACGGCCCCGTGCTGCTGCCCGA |
| | | CAACCACTACCTGAGCACCCAGTCCGCCCTGAGCAAAGACCCCAACGAGAAGCGCGATCACATGG |
| | | TCCTGCTGGAGTTCGTGACCGCCGCCGGGATCACTCTCGGCATGGACGAGCTGTACAAGAATCGCG |
| | | CTACTAACTTCAGCCTGCTGAAGCAGGCTGGAGACGTGGAGGAGAACCCTGGACCTATGGAAGAC |
| | | GCCAAAAACATAAAGAAAGGCCCGGCGCCATTCTATCCGCTGGAAGATGGAACCGCTGGAGAGCA |
| | | ACTGCATAAGGCTATGAAGAGATACGCCCTGGTTCCTGGAACAATTGCTTTTACAGATGCACATATC |
| | | GAGGTGGACATCACTTACGCTGAGTACTTCGAAATGTCCGTTCGGTTGGCAGAAGCTATGAAACGA |
| | | TATGGGCTGAATACAAATCACAGAATCGTCGTATGCAGTGAAAACTCTCTTCAATTCTTTATGCCGGT |
| | | GTTGGGCGCGTTATTTATCGGAGTTGCAGTTGCGCCCGCGAACGACATTTATAATGAACGTGAATTG |
| | | CTCAACAGTATGGGCATTTCGCAGCCTACCGTGGTGTTCGTTTCCAAAAAGGGGTTGCAAAAAATT |
| | | TTGAACGTGCAAAAAAAGCTCCCAATCATCCAAAAAATTATTATCATGGATTCTAAAACGGATTACC |
| | | AGGGATTTCAGTCGATGTACACGTTCGTCACATCTCATCTACCTCCCGGTTTTAATGAATACGATTTT |
| | | GTGCCAGAGTCCTTCGATAGGGACAAGACAATTGCACTGATCATGAACTCCTCTGGATCTACTGGTC |
| | | TGCCTAAAGGTGTCGCTCTGCCTCATAGAACTGCCTGCGTGAGATTCTCGCATGCCAGAGATCCTAT |
| | | TTTTGGCAATCAAATCATTCCGGATACTGCGATTTTAAGTGTTGTTCCATTCCATCACGGTTTTGGAA |
| | | TGTTTACTACACTCGGATATTTGATATGTGGATTTCGAGTCGTCTTAATGTATAGATTTGAAGAAGAG |
| | | CTGTTTCTGAGGAGCCTTCAGGATTACAAGATTCAAAGTGCGCTGCTGGTGCCAACCCTATTCTCCT |
| | | TCTTCGCCAAAAGCACTCTGATTGACAAATACGATTTATCTAATTTACACGAAATTGCTTCTGGTGGC |
| | | GCTCCCCTCTCTAAGGAAGTCGGGGAAGCGGTTGCCAAGAGGTTCCATCTGCCAGGTATCAGGCAA |
| | | GGATATGGGCTCACTGAGACTACATCAGCTATTCTGATTACACCCGAGGGGGATGATAAACCGGGCG |
| | | CGGTCGGTAAAGTTGTTCCATTTTTTGAAGCGAAGGTTGTGGATCTGGATACCGGGAAAACGCTGG |
| | | GCGTTAATCAAAGAGGCGAACTGTGTGTGAGAGGTCCTATGATTATGTCCGGTTATGTAAACAATCC |
| | | GGAAGCGACCAACGCCTTGATTGACAAGGATGGATGGCTACATTCTGGAGACATAGCTTACTGGGA |
| | | CGAAGACGAACACTTCTTCATCGTTGACCGCCTGAAGTCTCTGATTAAGTACAAAGGCTATCAGGT |
| | | GGCTCCCGCTGAATTGGAATCCATCTTGCTCCAACACCCCAACATCTTCGACGCAGGTGTCGCAGG |
| | | TCTTCCCGACGATGACGCCGGTGAACTTCCCGCCGCCGTTGTTGTTTTGGAGCACGGAAAGACGAT |
| | | GACGGAAAAAGAGATCGTGGATTACGTCGCCAGTCAAGTAACAACCGCGAAAAAGTTGCGCGGAG |
| | | GAGTTGTGTTTGTGGACGAAGTACCGAAAGGTCTTACCGGAAAACTCGACGCAAGAAAAATCAGA |
| | | GAGATCCTCATAAAGGCCAAGAAGGGCGGAAAGATCGCCGTGTAACTCGAGAATCAACCTCTGGAT |
| | | TACAAAATTTGTGAAAGATTGACTGGTATTCTTAACTATGTTGCTCCTTTTACGCTATGTGGATACGC |
| | | TGCTTTAATGCCTTTGTATCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCGTGGTGTGCACTGTG |
| | | TTTGCTGACGCAACCCCCACTGGTTGGGGCATTGCCACCACCTGTCAGCTCCTTTCCGGGACTTTC |
| | | GCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGGGAAGATGT |
| | | CTACTGAGCTGTGCGATCCCTGCTGGGGACTTTCCGCTGGGGACTTTCCGCTGGGGACTTTCCGCCT |
| | | TCAGCTAAGGAAGCTACCAATATTTAGAGGTACATTTTGTTCTAGAACAAAATGTACCGGTACATTTT |
| | | GTTCTGGTACATTTTGTTCTATTAAGGACAACGTAAGTATAGCGCATAGACACGTGGCGCCATCGGA |
| | | TCCCGGGCCCGTCGACTTAAGTGGCTTGTTGTCCACAACCATTAAACCTTAAAAGCTTTAAAAGCCT |
| | | TATATATTCTTTTTTTTCTTATAAAACTTAAAACCTTAGAGGCTATTTAAGTTGCTGATTTATATTAATT |
| | | TTATTGTTCAAACATGAGAGCTTAGTACGTGAAACATGAGAGCTTAGTACATTAGCCATGAGAGCTTA |
| | | GTACATTAGCCATGAGGGTTTAGTTCATTAAACATGAGAGCTTAGTACATTAAACATGAGAGCTTAG |
| | | TACATTAAACATGAGAGCTTAGTACATACTATCAACAGGTTGAACTGCTGATCTGTACAGTAGAATT |
| | | GGTAAAGAGAGTTGTGTAAAATATTGAGTTCGCACATCTTGTTGTCTGATTATTGATTTTTGGCGAAA |
| | | CCATTTGATCATATGACAAGATGTGTATCTACCTTAACTTAATGATTTTGATAAAAATCATTAGCGGCC |
| | | GC |
| 31. | pUC57- | TCTGGATCCACTGCTTAAATACGGACGAGGACAGGGCCCTGTCTCCTCAGCTTCAGGCACCACCAC |
| | CBA-hBGi- | TGACCTGGGACAGTGAATCGTAAGTACTAGCAGCTACAATCCAGCTACCATTCTGCTTTTATTTTATG |
| | DMD-4- | GTTGGGATAAGGCTGGATTATTCTGAGTCCAAGCTAGGCCCTTTTGCTAATCATGTTCATACCTCTTA |
| | WPRE3- | TCTTCCTCCCACAGCTCCTGGGCAACGTGCTGGTCTGTGTGCTGGCCCATCACTTTGGCAAAGAATT |
| | DTS | GCGATCGCCACCATGCTTTGGTGGGAAGAAGTCGAGGACTGCTACGAGCGCGAGGACGTGCAGAA |
| | | GAAAACCTTCACCAAATGGGTCAACGCCCAGTTCAGCAAGTTCGGCAAGCAGCACATCGAGAACC |
| | | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACGTGGCGCCATCGGATCCCGGGCCCGTCGACTGCAGAGGCCTGCATGCAAGCTTG |
| | | GCGTAATCATGGTCATAGCTGTTTCCTGTGTGAAATTGTTATCCGCTCACAATTCCACACAACATACG |
| | | AGCCGGAAGCATAAAGTGTAAAGCCTGGGGTGCCTAATGAGTGAGCTAACTCACATTAATTGCGTT |
| | | GCGCTCACTGCCCGCTTTCCAGTCGGGAAACCTGTCGTGCCAGCTGCATTAATGAATCGGCCAACG |
| | | CGCGGGGAGAGGCGGTTTGCGTATTGGGCGCTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAATC |
| | | AGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCA |
| | | AGTCAGAGGTGGCGAAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTCTCCCTTCGGGAAGCG |
| | | TGGCGCTTTCTCATAGCTCACGCTGTAGGTATCTCAGTTCGGTGTAGGTCGTTCGCTCCAAGCTGGG |
| | | CTGTGTGCACGAACCCCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTACACTAGAAGAACAGT |
| | | ATTTGGTATCTGCGCTCTGCTGAAGCCAGTTACCTTCGGAAAAAGAGTTGGTAGCTCTTGATCCGGC |
| | | AAACAAACCACCGCTGGTAGCGGTGGTTTTTTTGTTTGCAAGCAGCAGATTACGCGCAGAAAAAA |
| | | AGGATCTCAAGAAGATCCTTTGATCTTTTCTACGGGGTCTGACGCTCAGTGGAACGAAAACTCACG |
| | | TTAAGGGATTTTGGTCATGAGATTATCAAAAAGGATCTTCACCTAGATCCTTTTAAATTAAAAATGAA |
| | | GTTTTAAATCAATCTAAAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGAGACCCACGCTCACC |
| | | GGCTCCAGATTTATCAGCAATAAACCAGCCAGCCGGAAGGGCCGAGCGCAGAAGTGGTCCTGCAA |
| | | CTTTATCCGCCTCCATCCAGTCTATTAATTGTTGCCGGGAAGCTAGAGTAAGTAGTTCGCCAGTTAAT |
| | | AGTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCGG |
| | | TTAGCTCCTTCGGTCCTCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGCCCGGCGTCAATACGGGAT |
| | | AATACCGCGCCACATAGCAGAACTTTAAAAGTGCTCATCATTGGAAAACGTTCTTCGGGGCGAAAA |
| | | CTCTCAAGGATCTTACCGCTGTTGAGATCCAGTTCGATGTAACCCACTCGTGCACCCAACTGATCTT |
| | | CAGCATCTTTTACTTTCACCAGCGTTTCTGGGTGAGCAAAAACAGGAAGGCAAAATGCCGCAAAA |
| | | AAGGGAATAAGGGCGACACGGAAATGTTGAATACTCATACTCTTCCTTTTTCAATATTATTGAAGCAT |
| | | TTATCAGGGTTATTGTCTCATGAGCGGATACATATTTGAATGTATTTAGAAAAATAAACAAATAGGGG |
| | | TTCCGCGCACATTTCCCCGAAAAGTGCCACCTGACGTCTAAGAAACCATTATTATCATGACATTAAC |
| | | CTATAAAAATAGGCGTATCACGAGGCCCTTTCGTCTCGCGCGTTTCGGTGATGACGGTGAAAACCTC |
| | | TGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGC |
| | | AGATTGTACTGAGAGTGCACCATATGCGGTGTGAAATACCGCACAGATGCGTAAGGAGAAAATACC |
| | | GCATCAGGCGCCATTCGCCATTCAGGCTGCGCAACTGTTGGGAAGGGCGATCGGTGCGGGCCTCTT |
| | | CGCTATTACGCCAGCTGGCGAAAGGGGGATGTGCTGCAAGGCGATTAAGTTGGGTAACGCCAGGGT |
| | | TTTCCCAGTCACGACGTTGTAAAACGACGGCCAGTGAATTCGAGCTCGGTACCTCGCGAATGCATG |
| | | CGGCCGCCGTTACATAACTTACGGTAAATGGCCCGCCTGGCTGACCGCCCAACGACCCCCGCCCAT |
| | | TGACGTCAATAATGACGTATGTTCCCATAGTAACGCCAATAGGGACTTTCCATTGACGTCAATGGGT |
| | | GGAGTATTTACGGTAAACTGCCCACTTGGCAGTACATCAAGTGTATCATATGCCAAGTACGCCCCCT |
| | | ATTGACGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTCGAGGTGAGCCCCACGTTCTGC |
| | | TTCACTCTCCCCATCTCCCCCCCCTCCCCACCCCCAATTTTGTATTTATTTATTTTTTAATTATTTTGTG |
| | | CAGCGATGGGGGCGGGGGGGGGGGGGGCGCGCGCCAGGCGGGGCGGGGCGGGGCGAGGGGCGG |
| | | GGCGGGGCGAGGCGGAGAGGTGCGGCGGCAGCCAATCAGAGCGGCGCGCTCCGAAAGTTTCCTTT |
| | | TATGGCGAGGCGGCGGCGGCGGCGGCCCTATAAAAAGCGAAGCGCGCGGCGGGCGGGAGTC |
| 32. | pUC57- | TCTGGATCCACTGCTTAAATACGGACGAGGACAGGGCCCTGTCTCCTCAGCTTCAGGCACCACCAC |
| | MHCK7- | TGACCTGGGACAGTGAATCGTAAGTACTAGCAGCTACAATCCAGCTACCATTCTGCTTTTATTTTATG |
| | hBGi-DMD- | GTTGGGATAAGGCTGGATTATTCTGAGTCCAAGCTAGGCCCTTTTGCTAATCATGTTCATACCTCTTA |
| | 4-WPRE3- | TCTTCCTCCCACAGCTCCTGGGCAACGTGCTGGTCTGTGTGCTGGCCCATCACTTTGGCAAAGAATT |
| | DTS | GCGATCGCCACCATGCTTTGGTGGGAAGAAGTCGAGGACTGCTACGAGCGCGAGGACGTGCAGAA |
| | | GAAAACCTTCACCAAATGGGTCAACGCCCAGTTCAGCAAGTTCGGCAAGCAGCACATCGAGAACC |
| | | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACGTGGCGCCATCGGATCCCGGGCCCGTCGACTGCAGAGGCCTGCATGCAAGCTTG |
| | | GCGTAATCATGGTCATAGCTGTTTCCTGTGTGAAATTGTTATCCGCTCACAATTCCACACAACATACG |
| | | AGCCGGAAGCATAAAGTGTAAAGCCTGGGGTGCCTAATGAGTGAGCTAACTCACATTAATTGCGTT |
| | | GCGCTCACTGCCCGCTTTCCAGTCGGGAAACCTGTCGTGCCAGCTGCATTAATGAATCGGCCAACG |
| | | CGCGGGGAGAGGCGGTTTGCGTATTGGGCGCTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAATC |
| | | AGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCA |
| | | AGTCAGAGGTGGCGAAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTCTCCCTTCGGGAAGCG |
| | | TGGCGCTTTCTCATAGCTCACGCTGTAGGTATCTCAGTTCGGTGTAGGTCGTTCGCTCCAAGCTGGG |
| | | CTGTGTGCACGAACCCCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTACACTAGAAGAACAGT |
| | | ATTTGGTATCTGCGCTCTGCTGAAGCCAGTTACCTTCGGAAAAAGAGTTGGTAGCTCTTGATCCGGC |
| | | AAACAAACCACCGCTGGTAGCGGTGGTTTTTTTGTTTGCAAGCAGCAGATTACGCGCAGAAAAAA |
| | | AGGATCTCAAGAAGATCCTTTGATCTTTTCTACGGGGTCTGACGCTCAGTGGAACGAAAACTCACG |
| | | TTAAGGGATTTTGGTCATGAGATTATCAAAAAGGATCTTCACCTAGATCCTTTTAAATTAAAAATGAA |
| | | GTTTTAAATCAATCTAAAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGAGACCCACGCTCACC |
| | | GGCTCCAGATTTATCAGCAATAAACCAGCCAGCCGGAAGGGCCGAGCGCAGAAGTGGTCCTGCAA |
| | | CTTTATCCGCCTCCATCCAGTCTATTAATTGTTGCCGGGAAGCTAGAGTAAGTAGTTCGCCAGTTAAT |
| | | AGTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCGG |
| | | TTAGCTCCTTCGGTCCTCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGCCCGGCGTCAATACGGGAT |
| | | AATACCGCGCCACATAGCAGAACTTTAAAAGTGCTCATCATTGGAAAACGTTCTTCGGGGCGAAAA |
| | | CTCTCAAGGATCTTACCGCTGTTGAGATCCAGTTCGATGTAACCCACTCGTGCACCCAACTGATCTT |
| | | CAGCATCTTTTACTTTCACCAGCGTTTCTGGGTGAGCAAAAACAGGAAGGCAAAATGCCGCAAAA |
| | | AAGGGAATAAGGGCGACACGGAAATGTTGAATACTCATACTCTTCCTTTTTCAATATTATTGAAGCAT |
| | | TTATCAGGGTTATTGTCTCATGAGCGGATACATATTTGAATGTATTTAGAAAAATAAACAAATAGGGG |
| | | TTCCGCGCACATTTCCCCGAAAAGTGCCACCTGACGTCTAAGAAACCATTATTATCATGACATTAAC |
| | | CTATAAAAATAGGCGTATCACGAGGCCCTTTCGTCTCGCGCGTTTCGGTGATGACGGTGAAAACCTC |
| | | TGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGC |
| | | AGATTGTACTGAGAGTGCACCATATGCGGTGTGAAATACCGCACAGATGCGTAAGGAGAAAATACC |
| | | GCATCAGGCGCCATTCGCCATTCAGGCTGCGCAACTGTTGGGAAGGGCGATCGGTGCGGGCCTCTT |
| | | CGCTATTACGCCAGCTGGCGAAAGGGGGATGTGCTGCAAGGCGATTAAGTTGGGTAACGCCAGGGT |
| | | TTTCCCAGTCACGACGTTGTAAAACGACGGCCAGTGAATTCGAGCTCGGTACCTCGCGAATGCATC |
| | | TAGAAGCTGCATGTCTAAGCTAGACCCTTCAGATTAAAAATAACTGAGGTAAGGGCCTGGGTAGGG |
| | | GAGGTGGTGTGAGACGCTCCTGTCTCTCCTCTATCTGCCCATCGGCCCTTTGGGGAGGAGGAATGT |
| | | GCCCAAGGACTAAAAAAAGGCCATGGAGCCAGAGGGGCGAGGGCAACAGACCTTTCATGGGCAA |
| | | ACCTTGGGGCCCTGCTGTCTAGCATGCCCCACTACGGGTCTAGGCTGCCCATGTAAGGAGGCAAGG |
| | | CCTGGGGACACCCGAGATGCCTGGTTATAATTAACCCAGACATGTGGCTGCCCCCCCCCCCCCAAC |
| | | ACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCAC |
| 33. | NP- | AGCTCGGTACCTCGCGAATGCATCTAGAAGCTGCATGTCTAAGCTAGACCCTTCAGATTAAAAATAA |
| | MHCK7- | CTGAGGTAAGGGCCTGGGTAGGGGAGGTGGTGTGAGACGCTCCTGTCTCTCCTCTATCTGCCCATC |
| | hBGi- | GGCCCTTTGGGGAGGAGGAATGTGCCCAAGGACTAAAAAAAGGCCATGGAGCCAGAGGGGCGAG |
| | mafaDMD | GGCAACAGACCTTTCATGGGCAAACCTTGGGGCCCTGCTGTCTAGCATGCCCCACTACGGGTCTAG |
| | | GCTGCCCATGTAAGGAGGCAAGGCCTGGGGACACCCGAGATGCCTGGTTATAATTAACCCAGACAT |
| | | GTGGCTGCCCCCCCCCCCCCAACACCTGCTGCCTCTAAAAATAACCCTGTCCCTGGTGGATCCCCTG |
| | | CATGCGAAGATCTTCGAACAAGGCTGTGGGGGACTGAGGGCAGGCTGTAACAGGCTTGGGGGCCA |
| | | GGGCTTATACGTGCCTGGGACTCCCAAAGTATTACTGTTCCATGTTCCCGGCGAAGGGCCAGCTGTC |
| | | CCCCGCCAGCTAGACTCAGCACTTAGTTTAGGAACCAGTGAGCAAGTCAGCCCTTGGGGCAGCCC |
| | | ATACAAGGCCATGGGGCTGGGCAAGCTGCACGCCTGGGTCCGGGGTGGGCACGGTGCCCGGGCAA |
| | | CGAGCTGAAAGCTCATCTGCTCTCAGGGGCCCCTCCCTGGGGACAGCCCCTCCTGGCTAGTCACAC |
| | | CCTGTAGGCTCCTCTATATAACCCAGGGGCACAGGGGCTGCCCTCATTCTACCACCACCTCCACAGC |
| | | ACTCTGGATCCACTGCTTAAATACGGACGAGGACAGGGCCCTGTCTCCTCAGCTTCAGGCACCACC |
| | | ACTGACCTGGGACAGTGAATCGTAAGTACTAGCAGCTACAATCCAGCTACCATTCTGCTTTTATTTTA |
| | | TGGTTGGGATAAGGCTGGATTATTCTGAGTCCAAGCTAGGCCCTTTTGCTAATCATGTTCATACCTCT |
| | | TATCTTCCTCCCACAGCTCCTGGGCAACGTGCTGGTCTGTGTGCTGGCCCATCACTTTGGCAAAGAA |
| | | TTGCGATCGCCACCATGCTGTGGTGGGAGGAGGTGGAGGATTGCTACGAGAGGGAAGATGTCCAG |
| | | AAGAAAACCTTCACCAAGTGGGTGAACGCCCAGTTTAGCAAGTTCGGCAAGCAGCACATCGAGAA |
| | | CCTGTTTAGCGACCTGCAGGACGGCAGAAGGCTGCTGGACCTCCTGGAGGGGCTGACCGGCCAGA |
| | | AGCTGCCCAAGGAAAAGGGCAGCACCAGGGTGCACGCCCTGAATAACGTGAACAAGGCCCTGAG |
| | | AGTGCTGCAGAACAACAACGTGGACCTGGTGAACATCGGCAGCACCGACATCGTGGACGGCAACC |
| | | ACAAGCTGACCCTGGGCCTGATCTGGAATATCATCCTGCACTGGCAGGTGAAGAACGTGATGAAGA |
| | | ACATCATGGCCGGCCTGCAGCAGACCAACAGCGAGAAGATCCTGCTGAGCTGGGTGAGACAGAGC |
| | | ACCAGGAACTACCCCCAGGTGAACGTGATCAACTTCACCACCAGCTGGAGCGACGGGCTGGCCCT |
| | | GAACGCCCTGATCCACAGCCACAGGCCCGACCTGTTCGACTGGAACAGCGTGGTGTGCCAACAGA |
| | | GCGCCACGCAGAGGCTGGAGCACGCCTTCAACATCGCCAGATACCAGCTGGGCATCGAGAAGCTG |
| | | TTGGACCCCGAGGACGTGGCCACCACCTACCCCGACAAGAAGAGCATCCTGATGTACATCACCAGC |
| | | CTGTTCCAGGTGCTGCCCCAGCAGGTGAGCATCGAGGCCATCCAGGAGGTGGAGATGCTGCCCAG |
| | | GCCCCCCAAGGTGACAAAGGAGGAGCACTTCCAGCTGCACCACCAGATGCACTACAGCCAGCAGA |
| | | TCACGGTGTCACTGGCCCAGGGCTACGAGAGGACCAGCAGCCCCAAGCCCAGATTCAAGAGCTAC |
| | | GCCTACACCCAGGCCGCCTACGTGACCACCAGCGACCCCACCAGGTCCCCCTTCCCCAGCCAGCAC |
| | | CTGGAGGCCCCCGAGGATAAGAGTTTCGGCAGCAGCCTCATGGAGAGCGAAGTGAACCTGGACAG |
| | | ATACCAGACCGCCCTCGAAGAGGTGCTGAGCTGGCTGCTGAGCGCCGAGGATACCCTGCAGGCCC |
| | | AGGGCGAGATCAGCAACGACGTGGAGGTGGTGAAGGAGCAGTTCCACGCCCACGAGGGCTACATG |
| | | ATGGACCTGACCGCCCACCAGGGCAGGGTGGGCAACATCCTCCAACTGGGCAGCCAGCTGATCGG |
| | | CACCGGCAAGCTGAGCGAGGACGAGGAGACTGAGGTGCAGGAGCAGATGAACCTGCTGAACAGC |
| | | AGGTGGGAGTGCCTGAGGGTGGCCAGCATGGAGAAACAGTCCAACCTGCACAGGGTCCTGATGGA |
| | | CCTGCAGAACCAGAAGCTCAAGGAACTCAACGACTGGCTGACCAAGACCGAGGAGAGAACCAGG |
| | | AAGATGGAGGAGGAGCCCCTGGGCCCCGACCTGGAGGACCTGAAGAGGCAGGTGCAGCAGCACA |
| | | AGGTGCTGCAGGAGGATCTGGAGCAGGAACAGGTGAGGGTGAACAGCCTGACCCACATGGTGGT |
| | | GGTCGTGGACGAGAGCAGCGGCGACCATGCCACCGCCGCTCTGGAGGAGCAACTGAAGGTGCTG |
| | | GGCGACAGATGGGCCAACATCTGCAGATGGACCGAGGACAGGTGGGTGCTGCTCCAGGACATCCT |
| | | CCTGAAGTGGCAGAGGCTGACAGAGGAGCAGTGCCTGTTCAGCGCCTGGCTGAGCGAGAAGGAG |
| | | GACGTGGTGAACAGGATCCACACCACCGGCTTCAAGGACCAGAACGAGATGCTCAGCAGCCTGCA |
| | | AAAGCTGGCCGTCCTGAAGGCCGACTTGGAGAAAAAGAAGCAGAGCATGGACAAGCTGTACAGC |
| | | CTGAAGCAGGACCTCCTGAGCACCCTGAAGAACAAGAGTGTGACCCAGAAGATGGAGGCGTGGC |
| | | TGGAGAACTTCGCCAGGTGCTGGGACAACCTGGTGCAGAAGCTGGAGAAGAGCACCGCCCAGATT |
| | | AGCCAGGCCGTGACCACCACCCAGCCTAGCCTGACCCAGACCACGGTGATGGAGACTGTGACCAT |
| | | GGTGACCACCAGGGAGCAGATCCTGGTGAAGCACGCCCAGGAGGAACTCCCCCCCCCCCCGCCCC |
| | | AGAAGAAGAGACAGATCACCGTGGACTCCGAGATCAGGAAGAGGCTCGACGTGGACATCACCGA |
| | | ACTGCATAGCTGGATCACCAGAAGCGAGGCCGTGCTGCAGAGCCCCGAGTTCGCGATCTACAGGA |
| | | AGGAGGGCAACTTCAGCGACCTGAAGGAAAAGGTGAACGCCATCGAGAGGGAGAAGGCCGAGAA |
| | | GTTCAGGAAGCTCCAGGACGCCAGCAGGAGCGCCCAGGTGCTGGTGGAGCAGATGGTGAACGAG |
| | | GGGGTGAACGCCGACAGCATTAGGCAGGCCAGTGAGCAGCTGAATAGCAGGTGGATCGAGTTCTG |
| | | CCAGCTGCTCAGCGAGAGGCTCAACTGGCTGGAGTACCAGAACAACATTATCACCTTCTACAACCA |
| | | GCTGCAGCAGCTGGAGCAGATGACCACCACCGCCGAGAACTGGCTGAAGATCCAGCCCACCACCC |
| | | CCAGCGAGCCCACCGCCATCAAGAGTCAGCTGAAGATCTGCAAGGACGAGGTGAACAGACTGAGC |
| | | GCCCTGCAGCCCCAGATCGAGAGACTCAAGATCCAGTCCATCACCCTGAAGGAGAAGGGCCAGGG |
| | | CCCCATGTTCCTGGACGCCGACTTCGTGGCCTTCACCAACCACTTCAGCCAGGTGTTCAGCGACAT |
| | | GCAGGCCAGGGAGAAGGAGCTGCAGACCATCTTCGACACCCTGCCCCCCATGAGGTACCAAGAAA |
| | | CCATGAGCGCCATCAGGACCTGGGTGCAGCAGAGCGAGACTAAGCTGAGCATCCCCCAGCTGAGC |
| | | GTGACCGACTACGAGATCATGGAGCAGAGACTGGGCGAGCTGCAGGTGCTGCAAAGCAGCCTGCA |
| | | GGAGCAACAGAGCGGCCTGAACTACCTGAGCACCACCGTTAAGGAGATGAGCAAGAAGGCCCCC |
| | | AGCGAAATCAGCAGGAAGTACCAGCTGGAGTTCGAGGAAATCGAGACAAGGTGGAAGAAGCTCT |
| | | CCAGCCAGCTGGCCGAGCACTGCCAGAAGTTGGAGGAGCAAATGAACAAGCTGAGAAAAATCCA |
| | | GAACCACATCCAGACCCTGAAGAAGTGGATGGCCGAGGTGGACGTGTTCCTCAAGGAGGAGTGGC |
| | | CCGCCCTGGGCGACTCCGAGATTCTCAAAAAGCAGCTGAAGCAGTGTAGGCTGCTGGTGAACGAC |
| | | ATCCAGACCATCCAGCCCAGCCTGAACAGCGTGAACGAGGGCGGCCAGAAGATCAAGAACGAGG |
| | | CCGAGCCCGAGTTCGCCCTGAGGCTGGAGATCGAGCTGAAGGAGCTGAACACCCAGTGGGACCAC |
| | | ATGTGCCAGCAGGTGTACGCCAGGAAGGAGGCCCTGAAGGGCGGCCTGGAAAAGACCGTGAGCC |
| | | TGCAGAAGGACCTCAGCGAGATGCACGAGTGGATGACCCAGGCCGAGGAGGAGTACCTGGAGAG |
| | | GGACTTCGAGTACAAGACCCCCGACGAACTGCAGAAGGCCGTGGAAGAGATGAAGAGGGCCAAG |
| | | GAGGAGGCCCAACAAAAGGAGGCCAAGGTGAAGCTGCTGACAGAGAGCGTGAATAGCGTGATCG |
| | | CCCAGGCCCCTCCCGTGGCCCAGGAGGCGCTGAAGAAGGAACTGGAAACCCTGACCACCAACTAC |
| | | CAGTGGCTGTGCACCAGGCTGAACGGCAAGTGCAAGACCCTGGAGGAGGTGTGGGCCTGCTGGC |
| | | ACGAGCTGCTGAGCTACCTGGAAAAGGCCAACAAGTGGCTGAACGAGGTGGAGTTCAAGCTGAA |
| | | AACGACCGAGAACATCCCCGGCGGCGCCGAGGAGATCAGCGAGGTGCTGCACAGCCTGGAGAAC |
| | | CTGATGCAGCACAGCGAGGATAACCCCAACCAGATCAGGATCCTGGCCCAGACCCTGACCGACGG |
| | | CGGCGTGATGGACGAACTCATCAACGAGGAGCTGGAAACCTTCAACTCCAGATGGAGGGAGCTGC |
| | | ACGAGGAGGCCGTGAGGAGACAGAAGCTGCTCGAACAGAGCATCCAGAGCGCCCAGGAGATCGA |
| | | AAAGAGCCTGCACCTGATCCAGGAGAGCCTGACCTTCATCGACAAGCAGCTGACCGCCTACATCGC |
| | | CGACAAGGTGGACGCCGCCCAGATGCCCCAGGAGGCCCAGAAGATCCAGAGCGACCTGACCAGC |
| | | CACGAGATCAGCCTGGAGGAGATGAAGAAGCACAACCAGGGCAAGGAGGCCGCCCAGAGGGTGC |
| | | TGTCCCAGATCGACGTGGCCCAGAAGAAGCTGCAGGACGTGAGCATGAAGTTCCGGCTGTTCCAA |
| | | AAGCCCGCGAACTTCGAACAGAGGCTGCAGGAGAGCAAGATGATCCTGGACGAGGTCAAGATGCA |
| | | CCTGCCCGCCCTGGAAACCAAGAGTGTGGAGCAGGAGGTGGTGCAGAGCCAGCTCAACCACTGC |
| | | GTGAACCTGTACAAGAGCCTGAGCGAGGTGAAGAGTGAGGTCGAGATGGTGATTAAGACCGGGAG |
| | | GCAGATCGTGCAGAAAAAGCAGACCGAGAACCCCAAAGAGCTGGACGAGAGGGTGACCGCCCTG |
| | | AAACTGCACTACAACGAGCTGGGCGCCAAGGTGACCGAGAGGAAGCAGCAGCTTGAGAAGTGCC |
| | | TGAAGCTGTCCAGGAAGATGAGGAAGGAGATGAACGTGCTGACCGAGTGGCTGGCCGCCACCGAC |
| | | ATGGAGCTGACCAAGAGGAGCGCCGTGGAGGGGATGCCCAGCAACCTGGACAGCGAGGTGGCCT |
| | | GGGGCAAGGCCACCCAGAAGGAGATCGAGAAGCAGAAGGTGCACCTGAAGAGTATCACCGAGGT |
| | | GGGGGAGGCCCTCAAGACCGTGCTGGGCAAGAAGGAGACACTGGTGGAGGATAAGCTGAGCCTC |
| | | CTGAACAGCAACTGGATCGCCGTGACCAGCAGGGCCGAGGAGTGGCTGAACCTGCTGCTGGAGTA |
| | | TCAAAAGCACATGGAGACATTCGACCAGAACGTGGACCACATCACCAAGTGGATCATCCAGGCCG |
| | | ACACCCTGCTCGACGAGAGCGAAAAGAAGAAGAGCCAGCAGAAGGAGGATGTGTTGAAGAGGCT |
| | | GAAGGCCGAGCTGAACGACATCAGGCCCAAGGTGGACAGCACCAGGGACCAGGCCGCCAACCTG |
| | | ATGGCCAACAGGGGCGACCACTGCAGGAAGCTGGTGGAGCCCCAGATCAGCGAGCTGAACCACA |
| | | GGTTCGCCGCCATCAGCCACAGGATCAAGACCGGCAAGGCCAGTATCCCCCTCAAGGAGCTGGAG |
| | | CAGTTCAACAGCGACATCCAGAAGCTGCTGGAGCCTCTGGAGGCCGAGATCCAGCAGGGCGTGAA |
| | | CCTGAAGGAGGAGGATTTCAACAAGGACATGAACGAGGATAACGAGGGCACCGTGAAGGAGTTGC |
| | | TCCAGAGGGGCGACAACCTGCAGCAGAGGATCACCGATGAGAGAAAGAGGGAGGAGATCAAGAT |
| | | CAAGCAGCAACTGCTCCAGACCAAGCACAACGCCCTGAAGGACCTGAGATCCCAGAGGAGGAAG |
| | | AAGGCCCTGGAGATCAGCCACCAGTGGTACCAGTACAAGCGGCAGGCCGACGACCTGCTGAAGTG |
| | | CCTGGACGACATCGAGAAGAAGCTGGCCAGCCTGCCCGAGCCCAGGGACGAGAGAAAGATTAAG |
| | | GAGATCGACAGGGAGCTGCAGAAGAAGAAGGAGGAGCTGAACGCCGTGAGGAGGCAGGCCGAG |
| | | GGCCTGAGCGAAGATGGCGCCGCCATGGCCGTGGAGCCCACCCAGATCCAGCTGTCCAAGAGGTG |
| | | GAGGGAGATCGAGAGCAAGTTCGCCCAATTCAGGAGGCTGAACTTCGCCCAGATCCACACCGTGA |
| | | GGGAGGAAACCATGATGGTGATGACCGAGGATATGCCCCTGGAAATCAGCTACGTGCCCAGCACCT |
| | | ACCTGACCGAGATCACCCACGTGAGCCAGGCCCTGAGCGAAGTGGAGCAGCTGCTGAACGCCCCC |
| | | GACCTGTGCGCCAAGGACTTCGAGGATCTGTTTAAGCAGGAGGAGAGCCTCAAGAACATCAAGGA |
| | | CATCCTGCAGCAAAGCTCCGGCAGGATCGACATCATCCACAGCAAGAAAACCGCCGCCCTGCAGA |
| | | GCGCCACCCCCGTGGAGAGAGTCAAGCTGCAGGAAGCCCTGAGCCAGATCGACTTCCAGTGGGAG |
| | | AAGGTCAACAAGATGTACAAGGACAGACAGGGCAGGTTCGACAGAAGCGTGGAGAAGTGGAGGA |
| | | GGTTCCACTACGACATCAAGATCTTCAACCAGTGGCTGACCGAAGCCGAGCAGTTCCTGAGGAAA |
| | | ACCCAGATCCCCGAGAACTGGGAGCACGCCAAGTACAAGTGGTACCTGAAGGAACTCCAGGATGG |
| | | CATCGGCCAGAGGCAGACCGTGGTGAGGACCCTGAATGCCACCGGCGAGGAGATCATCCAGCAAA |
| | | GCAGCAAGACCGACGCCAGCATCCTGCAGGAGAAACTCGGCAGCCTGAACCTGAGGTGGCAGGA |
| | | GGTGTGCAAGCAGCTGAGCGAGAGGAAGAAGAGGCTGGAGGAGCAGAAGAACATCCTGAGCGAG |
| | | TTCCAGAGGGACCTGAACGAGTTCGTGCTGTGGCTGGAGGAGGCCGACAACATCGCCTCCATCCC |
| | | CCTGGAGCCCGGCAACGAGCAACAGCTGAAAGAGAAGCTGGAGCAGGTCAAGCTGCTGGTGGAG |
| | | GAGCTGCCCCTGAGGCAGGGCATCCTGAAGCAGCTGAACGAAACCGGCGGCCCCGTGCTGGTGAG |
| | | CGCCCCCATCAGCCCCGAGGAGCAGGACAAGCTGGAGAACAAGCTGAAGAGAACCAACCTGCAG |
| | | TGGATCAAGGTGAGCAGGGCCCTGCCCGAGAAGCAAGGCGAGATAGAGGCCCAGATCAAGGACCT |
| | | GGGCCAACTGGAAAAGAAGCTGGAGGATCTCGAAGAACAGCTGAACCACCTGCTGCTGTGGCTGA |
| | | GCCCCATCAGGAACCAGCTGGAGATCTATAACCAGCCCAACCAGGAGGGCCCCTTCGACATCAAGG |
| | | AGACAGAGATCGCCGTGCAGGCCAAGCAGCCCGACGTGGAGGGCATCCTGTCCAAGGGCCAGAG |
| | | GCTGTACAAGGAGAAGCCCGCCACACAGCCCGTGAAGAGAAAGCTGGAAGATCTCAGCAGCGAG |
| | | TGGAAAACCGTGAACCATCTGCTGCAGGAGCTGAGGGCCAAGCAACCCGACCTGGCCCCCGGCCT |
| | | GACCACCGTGGGCGCTAGCCCCAGCCAGACCGTGACCCTGGTGACCCAGCCCGTGGTGACCAAGG |
| | | AAACCGCCATCAGCAAGCTGGAGATGCCCAGCAGCCTGATGCTGGAGGTGCCTGCCCTGGCCGAC |
| | | TTCAACAGGGCCTGGACCGAGCTGACAGAGTGGCTGAGCCTGCTGGGCAGGGTGATCAAGAGCCA |
| | | GAGGGTGATGGTGGGCGACCTGGAAGATATCAACGAGATGATCATCAAGCAGAAGGCCACCGTGC |
| | | AGGACCTGGAGCAGAGAAGGCCCCAGCTGGAGGAGCTGATCACCGCCGCCCAGAACCTGAAGAA |
| | | TAAGACCAGCAACCAGGAGGCCAGGACAATCATCACCGACAGAATCGAGAGGATCCAGAACCAGT |
| | | GGGACGAAGTGCAGGAGCACCTGCAGAACAGGAGGCAGCAGCTCAACGAGATGCTGAAGGACAG |
| | | CACCCAGTGGCTGGAAGCCAAGGAGGAAGCTGAGCAGGTGCTGGGCCAGGCCAGGGCCAAGCTG |
| | | GAGAGCTGGAAGGAAGGCCCCTACACCATCGACGCCATCCAGAAGAAGATCACCGAAACCAAGCA |
| | | GCTGGCCAAGGACCTGAGGCAGTGGCAGATCAACGTGGACGTGGCCAACGACCTGGCCCTGAAGC |
| | | TGCTGCGGGACTACAGCGCCGACGACACCAGGAAGGTGCACATGATCACCGAAAACATCAACGCC |
| | | AGCTGGGGGAGCATCCACAAGAGGGTGAGCGAGCAGGAGGCCGCGCTGGAGGAAACCCACAGGC |
| | | TGCTGCAGCAGTTCCCCCTGGACCTCGAAAAGTTCCTGGCCTGGCTGACCGAGGCCGAGACAACC |
| | | GCCAACGTGCTGCAGGACGCTACCAGGAAGGAGAGGCTGCTGGAGGATAGCAAGGGCGTGAAGG |
| | | AACTCATGAAGCAGTGGCAGGACCTGCAGGGCGAGATTGAGGCCCACACCGACGTGTACCACAAC |
| | | CTGGACGAGAACAGCCAGAAGGTCCTGAGGAGCTTGGAGGGCAGCGACGACGCCGTGCTGCTGC |
| | | AGAGAAGGCTGGACAACATGAACTTCAAGTGGAGCGAGCTGAGGAAGAAGTCCCTGAACATTAGA |
| | | AGCCACCTGGAGGCTAGTAGCGACCAGTGGAAAAGGCTGCACCTGAGCCTCCAGGAGCTGCTCGT |
| | | GTGGCTGCAGCTGAAGGACGACGAGCTGAGCAGACAGACCCCCATCGGCGGCGACTTCCCCGCCG |
| | | TGCAGAAGCAGAACGACGTGCACAGGGCTTTCAAGAGGGAGCTGAAAACCAAGGAGCCCGTGAT |
| | | CATGAGCACCCTGGAGACAGTGAGGATCTTCCTCACCGAGCAGCCCCTGGAGGGCCTGGAGAAGC |
| | | TGTACCAGGAGCCCAGGGAGCTGCCCCCCGAGGAGAGAGCGCAGAACGTGACCAGACTGCTGAG |
| | | GAAGCAGGCCGAGGAAGTGAACACCGAGTGGGAGAAGCTGAACCTGCACAGCACCGACTGGCAG |
| | | AGGAAGATCGACGAAACCCTGGAGAGGCTGCAAGAGCTGCAGGAGGCCATGGACGAGCTGGACC |
| | | TGAAACTGAGACAGGCCGAGGTGATCAAGGGCAGCTGGCAGCCCGTCGGCGACCTGCTGATCGAC |
| | | AGCCTCCAGGACCACCTGGAAAAAGTGAAGGCCCTGAGGGGCGAGATCGCCCCTCTGAAGGAGA |
| | | ACGTGAGCCACGTGAACGACCTCGCCAGACAGCTGACCACCCTGGGCATCCAGCTGAGCCCCTAC |
| | | AACCTCAGCACCCTGGAAGATCTGAACACCAGGTGGAAGCTGCTGCAGGTGGCCGTCGAGGACAG |
| | | GGTGAGGCAGCTCCACGAGGCCCACAGGGACTTCGGCCCCGCCAGCCAGCACTTCCTGAGCACCA |
| | | GCGTGCAGGGCCCCTGGGAGAGAGCCATCAGCCCGAACAAGGTGCCCTACTACATCAACCACGAA |
| | | ACCCAGACCACCTGCTGGGACCACCCCAAGATGACCGAGCTGTACCAGAGCCTGGCCGACCTGAA |
| | | CAATGTGAGATTCTCCGCCTACAGAACCGCCATGAAGCTGAGGAGGCTCCAGAAGGCCCTGTGCCT |
| | | GGACCTGCTGAGCCTGAGCGCCGCCTGCGACGCCCTGGACCAGCACAACCTGAAGCAAAACGACC |
| | | AGCCCATGGACATCCTCCAGATCATCAACTGCCTGACCACCATCTACGACAGACTGGAGCAGGAGC |
| | | ACAACAACCTGGTGAACGTGCCCCTGTGCGTGGACATGTGCCTGAACTGGCTGCTGAACGTGTACG |
| | | ACACCGGCAGGACCGGCAGAATCAGAGTGCTGAGCTTCAAGACCGGGATCATCAGCCTGTGCAAG |
| | | GCCCACCTGGAGGACAAGTACAGGTACCTGTTCAAGCAGGTCAGCAGCAGCACCGGCTTCTGCGA |
| | | CCAGAGACGGCTGGGCCTGCTGCTGCACGACAGCATCCAGATCCCCAGACAGCTGGGCGAGGTGG |
| | | CCAGCTTCGGCGGCAGCAACATCGAGCCCAGCGTGAGAAGCTGCTTCCAGTTCGCCAACAACAAG |
| | | CCCGAGATCGAGGCCGCCCTGTTCCTGGACTGGATGAGGCTGGAGCCCCAAAGCATGGTGTGGCT |
| | | GCCCGTGCTGCACAGGGTGGCCGCCGCCGAGACAGCCAAGCACCAGGCCAAGTGCAACATCTGCA |
| | | AGGAGTGCCCCATCATCGGCTTCAGGTACAGGAGCCTGAAGCACTTCAACTACGACATCTGCCAGA |
| | | GCTGCTTCTTCAGCGGCAGGGTGGCCAAGGGCCACAAGATGCACTACCCCATGGTGGAGTACTGCA |
| | | CCCCCACCACCAGCGGCGAGGATGTGAGAGACTTCGCCAAGGTGCTGAAAAACAAGTTCAGGACC |
| | | AAGAGGTACTTCGCCAAGCACCCCAGAATGGGCTACCTGCCCGTGCAGACCGTCCTGGAGGGCGA |
| | | CAACATGGAGACACCCGTGACCCTGATCAACTTCTGGCCCGTGGACAGCGCCCCCGCCAGCAGCC |
| | | CTCAGCTGAGCCACGACGACACCCACAGCAGGATCGAGCACTACGCCAGCAGGCTGGCCGAGATG |
| | | GAGAACAGCAACGGCAGCTACCTGAACGACAGCATCAGCCCCAACGAGAGCATCGACGACGAGC |
| | | ACCTGCTGATCCAGCACTACTGCCAGAGCCTGAACCAGGACAGCCCCCTGAGCCAGCCCAGGTCC |
| | | CCCGCCCAGATCCTGATCAGCCTCGAAAGCGAGGAGAGGGGCGAGCTGGAGAGAATCCTGGCCGA |
| | | TCTGGAGGAGGAGAACAGGAACCTGCAGGCCGAGTACGACAGGCTGAAGCAGCAGCACGAGCAC |
| | | AAGGGCCTGAGCCCCCTGCCCAGCCCCCCCGAGATGATGCCCACCAGCCCCCAGAGCCCCAGGGA |
| | | CGCCGAGCTGATCGCCGAGGCGAAGCTGCTGAGGCAGCACAAGGGCAGGCTGGAGGCCAGGATG |
| | | CAGATCCTGGAGGACCACAACAAGCAGCTGGAGAGCCAGCTGCACAGACTGAGGCAGCTGCTTG |
| | | AGCAGCCCCAGGCCGAAGCCAAGGTGAACGGCACCACCGTCAGCAGCCCCAGCACCAGCCTGCA |
| | | GAGGAGCGACAGCAGCCAGCCCATGCTGCTGAGGGTGGTGGGCAGCCAGACCAGCGACAGCATG |
| | | GGCGAGGAGGATGTGCTGAGCCCCCCCCAGGACACCAGCACCGGCCTGGAGGAAGTGATGGAGC |
| | | AGCTGAACAACAGCTTCCCCAGCAGCAGGGGCAGGAACACCCCCGGCAAGCCCATGAGGGAGGA |
| | | CACCATGGATTACAAGGATGACGATGACAAGTGATAACTCGAGAATCAACCTCTGGATTACAAAATT |
| | | TGTGAAAGATTGACTGGTATTCTTAACTATGTTGCTCCTTTTACGCTATGTGGATACGCTGCTTTAATG |
| | | CCTTTGTATCATGCTATTGCTTCCCGTATGGCTTTCATTTTCTCCTCCTTGTATAAATCCTGGTTGCTGT |
| | | CTCTTTATGAGGAGTTGTGGCCCGTTGTCAGGCAACGTGGCGTGGTGTGCACTGTGTTTGCTGACG |
| | | CAACCCCCACTGGTTGGGGCATTGCCACCACCTGTCAGCTCCTTTCCGGGACTTTCGCTTTCCCCCT |
| | | CCCTATTGCCACGGCGGAACTCATCGCCGCCTGCCTTGCCCGCTGCTGGACAGGGGCTCGGCTGTT |
| | | GGGCACTGACAATTCCGTGGGGTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAATAAAATGAGGAAATTGCATCG |
| | | CATTGTCTGAGTAGGTGTCATTCTATTCTGGGGGGTGGGGTGGGGCAGGACAGCAAGGGGGAGGAT |
| | | TGGGAAGACAATAGCAGGCATGCTGGGGATGCGGTGGGCTCTATGGGAAGATGTCTACTGAGCTGT |
| | | GCGATCCCTGCTGGGGACTTTCCGCTGGGGACTTTCCGCTGGGGACTTTCCGCCTTCAGCTAAGGA |
| | | AGCTACCAATATTTAGAGGTACATTTTGTTCTAGAACAAAATGTACCGGTACATTTTGTTCTGGTACA |
| | | TTTTGTTCTATTAAGGACCGGGCGCGCCGTTTAAACTGGCTTGTTGTCCACAACCATTAAACCTTAA |
| | | AAGCTTTAAAAGCCTTATATATTCTTTTTTTTCTTATAAAACTTAAAACCTTAGAGGCTATTTAAGTTG |
| | | CTGATTTATATTAATTTTATTGTTCAAACATGAGAGCTTAGTACGTGAAACATGAGAGCTTAGTACAT |
| | | TAGCCATGAGAGCTTAGTACATTAGCCATGAGGGTTTAGTTCATTAAACATGAGAGCTTAGTACATTA |
| | | AACATGAGAGCTTAGTACATTAAACATGAGAGCTTAGTACATACTATCAACAGGTTGAACTGCTGAT |
| | | CTGTACAGTAGAATTGGTAAAGAGAGTTGTGTAAAATATTGAGTTCGCACATCTTGTTGTCTGATTAT |
| | | TGATTTTTGGCGAAACCATTTGATCATATGACAAGATGTGTATCTACCTTAACTTAATGATTTTGATAA |
| | | AAATCATTAGGCGCGCCGAAGAGCG |
| 34. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 4- | TGCCCAAGGACTAAAAAAAGGCCATGGAGCCAGAGGGGCGAGGGCAACAGACCTTTCATGGGCA |
| | | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTT |
| | | TGGTGGGAAGAAGTCGAGGACTGCTACGAGCGCGAGGACGTGCAGAAGAAAACCTTCACCAAAT |
| | | GGGTCAACGCCCAGTTCAGCAAGTTCGGCAAGCAGCACATCGAGAACCTGTTCAGCGACCTGCAG |
| | | GATGGCAGAAGGCTGCTGGATCTGCTGGAAGGCCTGACAGGACAGAAGCTGCCCAAAGAGAAGG |
| | | GCAGCACAAGAGTGCACGCCCTGAACAACGTGAACAAGGCCCTGAGAGTGCTGCAGAACAACAA |
| | | CGTGGACCTGGTCAACATCGGCAGCACCGACATCGTGGACGGCAACCACAAACTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATTCTGCTGAGCTGGGTCCGACAGAGCACCCGGAATTACCCTCAA |
| | | GTGAACGTGATCAACTTCACCACCTCTTGGAGCGACGGACTGGCCCTGAATGCCCTGATCCACAGC |
| | | CACAGACCTGACCTGTTCGACTGGAACAGCGTCGTGTGTCAGCAGAGCGCCACACAGAGGCTGGA |
| | | ACACGCCTTCAATATCGCCAGATACCAGCTGGGCATCGAGAAACTGCTGGACCCCGAGGATGTGGA |
| | | CACCACCTATCCTGACAAGAAATCCATCCTCATGTACATCACCAGCCTGTTCCAGGTGCTGCCCCAG |
| | | CAGGTTTCCATCGAGGCCATTCAAGAGGTCGAGATGCTGCCCAGACCTCCTAAAGTGACCAAAGAG |
| | | GAACACTTCCAGCTGCACCACCAGATGCACTACTCTCAGCAGATCACCGTGTCTCTGGCCCAGGGC |
| | | TACGAGAGAACAAGCAGCCCCAAGCCTCGGTTCAAGAGCTACGCCTATACACAGGCCGCCTACGTG |
| | | ACCACCAGCGATCCTACAAGAAGCCCATTTCCTAGCCAGCATCTGGAAGCCCCTGAGGACAAGAGC |
| | | TTTGGCAGCAGCCTGATGGAAAGCGAAGTGAACCTGGACCGCTACCAGACAGCCCTGGAAGAGGT |
| | | TCTGAGCTGGCTGCTGTCTGCCGAGGATACACTGCAGGCTCAGGGCGAGATCAGCAACGACGTGG |
| | | AAGTGGTCAAGGACCAGTTTCACACCCACGAGGGCTACATGATGGACCTGACAGCCCACCAGGGC |
| | | AGAGTGGGCAATATTCTGCAGCTGGGCTCCAAGCTGATCGGCACAGGCAAGCTGAGCGAGGACGA |
| | | AGAGACAGAGGTGCAAGAGCAGATGAACCTGCTGAACAGCAGATGGGAGTGTCTGAGAGTGGCC |
| | | AGCATGGAAAAGCAGAGCAACCTGCACCGGGTGCTGATGGATCTCCAGAACCAGAAGCTGAAAGA |
| | | GCTGAACGACTGGCTGACCAAGACCGAGGAACGGACCCGGAAGATGGAAGAGGAACCTCTGGGA |
| | | CCCGACCTGGAAGATCTGAAAAGACAGGTGCAGCAGCATAAGGTGCTGCAAGAGGACCTCGAACA |
| | | AGAGCAAGTGCGCGTGAACAGCCTGACACACATGGTGGTGGTCGTGGATGAGAGCAGCGGAGATC |
| | | ATGCCACAGCCGCTCTGGAAGAACAGCTGAAGGTGCTGGGAGACAGATGGGCCAATATCTGCCGG |
| | | TGGACCGAGGATAGATGGGTGCTGCTCCAGGACATCCTGCTGAAGTGGCAGCGGCTGACAGAGGA |
| | | ACAGTGCCTGTTTAGCGCCTGGCTGTCCGAGAAAGAGGACGCCGTCAACAAGATCCACACCACCG |
| | | GCTTCAAGGATCAGAATGAGATGCTGAGCAGCCTGCAGAAACTGGCCGTGCTGAAGGCTGACCTG |
| | | GAAAAGAAAAAGCAGTCCATGGGCAAGCTGTACTCCCTGAAGCAGGACCTGCTGAGCACACTGAA |
| | | GAACAAGTCTGTGACCCAGAAAACCGAGGCCTGGCTGGACAACTTCGCCCGGTGTTGGGATAACC |
| | | TGGTGCAGAAGCTGGAAAAGTCCACCGCTCAGATCTCTCAGGCCGTGACCACAACACAGCCTTCT |
| | | CTGACCCAGACCACCGTGATGGAAACAGTGACCACAGTGACAACCCGCGAGCAGATCCTGGTCAA |
| | | GCACGCCCAAGAAGAACTGCCTCCTCCACCTCCTCAGAAGAAACGGCAGATCACAGTGGACAGCG |
| | | AGATCCGGAAGAGACTGGACGTGGACATCACCGAGCTGCACAGCTGGATCACCAGATCCGAAGCC |
| | | GTGCTGCAGTCCCCTGAGTTCGCCATCTTCAGAAAAGAGGGCAACTTCTCCGACCTGAAAGAGAA |
| | | AGTCAACGCCATCGAGAGAGAGAAGGCCGAGAAGTTCCGGAAGCTGCAGGACGCCTCTAGATCTG |
| | | CCCAGGCTCTGGTGGAACAGATGGTCAACGAAGGCGTGAACGCCGACAGCATCAAGCAGGCCTCA |
| | | GAGCAGCTGAACTCCCGGTGGATCGAGTTCTGTCAGCTCCTGAGCGAGAGACTGAACTGGCTGGA |
| | | ATACCAGAACAATATCATTGCCTTCTACAACCAGCTCCAGCAGCTCGAACAGATGACCACCACAGC |
| | | CGAGAATTGGCTGAAGATCCAGCCTACCACACCTAGCGAGCCCACCGCCATTAAGAGCCAGCTGAA |
| | | AATCTGCAAGGACGAAGTGAATCGGCTGAGCGATCTGCAGCCCCAGATCGAAAGACTGAAAATCC |
| | | AGTCTATCGCCCTCAAAGAGAAAGGACAGGGCCCCATGTTCCTGGACGCCGATTTTGTGGCCTTTA |
| | | CCAACCACTTCAAACAGGTGTTCTCCGACGTGCAGGCCCGGGAAAAAGAGCTGCAGACCATCTTC |
| | | GACACCCTGCCTCCAATGAGATACCAAGAGACAATGAGCGCCATCCGGACCTGGGTGCAGCAAAG |
| | | CGAGACAAAGCTGAGCATCCCACAGCTGAGCGTGACCGACTACGAGATCATGGAACAGAGACTGG |
| | | GCGAACTGCAGGCCCTCCAGTCTAGCCTGCAAGAACAGCAGTCCGGCCTGTACTACCTGAGCACA |
| | | ACCGTGAAAGAGATGAGCAAGAAGGCCCCTAGCGAGATCTCCCGGAAGTACCAGAGCGAGTTCGA |
| | | AGAGATCGAAGGCCGGTGGAAGAAGCTGTCTAGCCAGCTGGTTGAGCACTGCCAGAAACTCGAAG |
| | | AACAAATGAACAAGCTGCGCAAGATCCAGAATCACATCCAGACGCTGAAGAAATGGATGGCCGAG |
| | | GTGGACGTGTTCCTGAAAGAAGAGTGGCCCGCTCTGGGCGACTCCGAGATTCTGAAGAAACAGCT |
| | | CAAACAGTGCCGGCTGCTGGTGTCCGATATCCAGACAATCCAGCCAAGCCTGAACTCCGTGAATGA |
| | | AGGCGGCCAGAAGATCAAGAACGAGGCCGAGCCTGAGTTTGCCAGCAGACTGGAAACCGAACTG |
| | | AAAGAACTCAACACCCAGTGGGACCACATGTGCCAGCAAGTGTACGCCCGGAAAGAGGCCCTGAA |
| | | AGGCGGACTCGAAAAGACTGTGTCTCTGCAGAAAGACCTGTCTGAGATGCACGAGTGGATGACCC |
| | | AGGCCGAAGAGGAATACCTGGAACGGGACTTCGAGTACAAGACCCCTGATGAGCTGCAAAAAGCC |
| | | GTCGAGGAAATGAAGCGGGCCAAAGAAGAGGCCCAGCAGAAAGAAGCCAAAGTCAAGCTGCTGA |
| | | CCGAGTCCGTGAATAGCGTTATCGCTCAGGCCCCTCCTGTGGCTCAAGAGGCTCTCAAGAAAGAAC |
| | | TGGAAACTCTGACCACCAACTACCAGTGGCTGTGCACCAGACTGAACGGCAAGTGCAAGACACTG |
| | | GAAGAAGTGTGGGCCTGCTGGCACGAACTGCTGTCCTATCTGGAAAAGGCCAACAAGTGGCTGAA |
| | | CGAGGTGGAATTCAAGCTGAAAACCACCGAGAACATCCCTGGCGGCGCTGAAGAGATTAGCGAGG |
| | | TGCTGGACAGCCTGGAAAACCTGATGAGACACAGCGAGGACAACCCCAATCAGATCAGAATCCTG |
| | | GCTCAGACCCTGACCGATGGCGGCGTGATGGACGAGCTGATCAACGAGGAACTCGAAACCTTCAA |
| | | CAGCCGGTGGCGGGAACTGCACGAGGAAGCTGTTCGGAGGCAGAAGTTGCTGGAACAGTCTATCC |
| | | AGAGCGCACAAGAAACCGAGAAGTCCCTGCACCTGATCCAAGAGAGCCTGACCTTCATCGACAAG |
| | | CAGCTGGCCGCTTATATCGCCGACAAGGTGGACGCTGCTCAGATGCCCCAAGAGGCACAGAAGATT |
| | | CAGAGCGACCTGACCAGCCACGAGATTAGCCTGGAAGAAATGAAGAAGCACAACCAGGGCAAAG |
| | | AGGCCGCTCAGAGAGTCCTGAGCCAGATCGATGTGGCACAGAAAAAGCTCCAGGATGTGTCCATG |
| | | AAGTTTCGGCTGTTTCAGAAGCCCGCCAACTTCGAGCAGAGACTGCAAGAGTCCAAGATGATCCT |
| | | GGACGAAGTCAAAATGCATCTGCCCGCACTGGAAACAAAGTCCGTGGAACAAGAAGTGGTGCAGT |
| | | CCCAGCTGAACCACTGCGTGAACCTGTACAAGAGCCTGTCCGAAGTGAAGTCTGAGGTGGAAATG |
| | | GTCATCAAGACCGGCAGGCAGATCGTCCAGAAGAAGCAGACCGAGAATCCCAAAGAACTCGACG |
| | | AGAGAGTGACCGCTCTGAAGCTGCACTACAATGAGCTGGGCGCCAAAGTGACCGAGCGGAAGCA |
| | | ACAGCTTGAGAAGTGCCTGAAACTGTCTCGGAAGATGCGGAAAGAAATGAACGTGCTGACAGAGT |
| | | GGCTGGCCGCCACCGATATGGAACTGACTAAGAGAAGCGCCGTGGAAGGCATGCCCAGCAACCTG |
| | | GATAGTGAAGTGGCCTGGGGCAAAGCCACTCAGAAAGAGATTGAGAAGCAGAAGGTCCACCTGA |
| | | AGTCCATCACCGAAGTGGGCGAAGCCCTGAAAACCGTGCTGGGAAAGAAAGAGACACTGGTCGA |
| | | GGACAAGCTGTCCCTGCTGAATTCCAACTGGATCGCCGTGACCTCCAGAGCTGAGGAATGGCTGAA |
| | | TCTGCTGCTTGAGTACCAGAAACACATGGAAACGTTCGACCAGAACGTCGACCACATCACAAAGT |
| | | GGATCATCCAGGCTGATACCCTGCTGGACGAGTCCGAGAAGAAGAAACCCCAACAAAAAGAAGAT |
| | | GTGCTGAAGCGGCTGAAAGCCGAGCTGAATGACATCCGGCCTAAGGTGGACAGCACCAGAGATCA |
| | | GGCAGCCAACCTGATGGCCAACAGAGGCGACCACTGTCGGAAGCTCGTGGAACCTCAGATCAGCG |
| | | AGCTTAACCACAGATTTGCCGCCATCAGCCACCGGATCAAGACAGGCAAGGCCAGCATTCCTCTCA |
| | | AAGAGCTTGAGCAGTTCAACAGCGACATCCAAAAGCTGCTCGAACCCCTGGAAGCCGAGATCCAA |
| | | CAGGGCGTCAACCTCAAAGAAGAAGATTTCAACAAGGACATGAACGAGGACAATGAGGGCACCG |
| | | TCAAAGAACTGCTGCAGCGGGGCGATAATCTGCAGCAGCGGATTACCGATGAGCGCAAGCGGGAA |
| | | GAGATCAAGATTAAGCAGCAGCTGCTGCAGACCAAGCACAACGCCCTGAAGGACCTGCGGAGCCA |
| | | GAGAAGAAAGAAGGCCCTGGAAATCAGCCACCAGTGGTATCAGTACAAGCGGCAGGCCGACGAC |
| | | CTGCTGAAGTGCCTGGATGACATCGAGAAGAAGCTGGCCTCTCTGCCCGAGCCTAGGGACGAGAG |
| | | AAAGATCAAAGAGATCGACCGCGAGCTGCAGAAAAAGAAAGAGGAACTGAACGCCGTCCGCAGA |
| | | CAGGCCGAAGGACTTTCTGAAGATGGCGCCGCTATGGCCGTGGAACCCACACAGATTCAGCTGAG |
| | | CAAGCGGTGGCGGGAAATCGAGAGCAAGTTCGCCCAGTTCCGGCGGCTGAATTTCGCCCAGATCC |
| | | ACACCGTGCGGGAAGAGACAATGATGGTCATGACAGAGGACATGCCCCTTGAGATCAGCTACGTGC |
| | | CCAGCACCTACCTGACCGAGATCACCCATGTGTCTCAGGCCCTGCTGGAAGTGGAACAGCTGCTGA |
| | | ATGCCCCTGACCTGTGCGCCAAGGACTTCGAGGACCTGTTCAAGCAAGAGGAAAGCCTGAAGAAC |
| | | ATCAAGGACAGCCTGCAGCAGAGCAGCGGCCGGATCGATATCATCCACAGCAAGAAAACCGCCGC |
| | | ACTGCAGAGCGCCACACCTGTGGAAAGAGTGAAGCTGCAAGAGGCCCTGAGCCAGCTGGATTTCC |
| | | AGTGGGAGAAAGTGAACAAGATGTACAAGGACCGGCAGGGCAGATTCGACCGCAGCGTTGAAAA |
| | | GTGGCGGCGGTTCCACTACGACATCAAGATCTTCAACCAGTGGCTGACCGAGGCCGAGCAGTTCCT |
| | | GAGAAAGACACAGATCCCCGAGAACTGGGAGCACGCCAAGTACAAGTGGTATCTGAAAGAGCTGC |
| | | AGGACGGCATCGGCCAGAGACAGACAGTCGTGCGGACACTGAATGCCACCGGCGAGGAAATCATC |
| | | CAGCAGTCCAGCAAGACCGACGCCAGCATCCTGCAAGAGAAGCTGGGCAGCCTGAACCTGCGGTG |
| | | GCAAGAAGTGTGCAAGCAGCTGAGCGACCGGAAGAAGCGGCTGGAAGAACAGAAGAATATCCTG |
| | | AGCGAGTTCCAGCGGGACCTGAACGAGTTCGTGCTGTGGCTCGAAGAGGCCGACAATATCGCCAG |
| | | CATTCCCCTGGAACCTGGCAAAGAGCAGCAGCTGAAAGAAAAGCTGGAACAAGTGAAACTGCTG |
| | | GTCGAGGAACTGCCCCTGAGACAGGGAATCCTGAAACAGCTGAACGAGACAGGCGGCCCTGTGCT |
| | | GGTGTCTGCCCCTATTTCTCCCGAGGAACAGGACAAGCTGGAAAACAAGCTGAAGCAGACCAACC |
| | | TGCAGTGGATCAAGGTGTCCAGAGCACTGCCTGAGAAGCAGGGCGAGATCGAGGCCCAGATCAAG |
| | | GACCTGGGACAGCTCGAAAAGAAGCTTGAGGACCTCGAAGAACAGCTCAACCATCTGCTGCTTTG |
| | | GCTGAGCCCCATCCGGAACCAGCTGGAAATCTACAACCAGCCTAATCAAGAGGGCCCCTTCGACGT |
| | | GAAAGAAACCGAGATTGCCGTGCAGGCCAAGCAGCCCGATGTGGAAGAGATCCTGTCTAAGGGCC |
| | | AGCACCTGTACAAAGAGAAGCCCGCCACACAGCCCGTGAAGCGGAAACTGGAAGATCTGAGCAG |
| | | CGAGTGGAAGGCCGTGAACCGGCTGCTGCAAGAACTGAGAGCCAAACAGCCTGATCTGGCCCCTG |
| | | GCCTGACAACAATTGGCGCCTCTCCTACACAGACCGTGACACTGGTCACACAGCCTGTGGTCACCA |
| | | AAGAGACAGCCATCAGCAAACTGGAAATGCCCAGCAGTCTGATGCTCGAAGTGCCCGCACTGGCC |
| | | GACTTCAATAGAGCCTGGACCGAGCTGACCGACTGGCTCAGTCTGCTGGACCAAGTCATCAAGTCC |
| | | CAGAGAGTGATGGTCGGAGATCTTGAGGACATCAACGAGATGATCATTAAGCAGAAAGCCACCATG |
| | | CAGGACCTTGAGCAGAGAAGGCCCCAGCTTGAAGAACTGATCACCGCCGCTCAGAACCTGAAAAA |
| | | CAAGACCAGCAATCAAGAAGCCCGGACCATCATCACCGACCGGATCGAGAGAATCCAGAACCAGT |
| | | GGGACGAAGTGCAAGAGCATCTGCAGAACAGACGGCAGCAGCTCAATGAGATGCTGAAGGACAG |
| | | CACACAGTGGCTGGAAGCCAAAGAAGAAGCCGAGCAGGTTCTCGGACAGGCCAGAGCTAAGCTG |
| | | GAATCTTGGAAAGAGGGACCCTACACCGTCGACGCCATCCAGAAGAAGATCACCGAGACAAAGCA |
| | | GCTGGCCAAGGATCTGAGACAGTGGCAGACCAATGTGGACGTGGCCAACGACCTGGCTCTGAAGC |
| | | TGCTGAGAGACTACAGCGCCGACGACACCAGAAAGGTGCACATGATCACCGAAAACATCAACGCC |
| | | TCTTGGCGGAGCATCCACAAGCGGGTGTCAGAGAGAGAAGCCGCTCTGGAAGAAACACATAGACT |
| | | GCTCCAGCAGTTCCCACTCGACCTGGAAAAGTTCCTGGCCTGGCTGACAGAAGCCGAAACCACCG |
| | | CTAACGTGCTCCAGGATGCCACCAGAAAAGAGCGGCTGTTGGAGGACAGCAAGGGCGTCAAAGA |
| | | ACTCATGAAGCAGTGGCAGGATCTGCAAGGGGAGATTGAAGCCCACACCGACGTGTACCACAACC |
| | | TGGACGAGAACAGCCAGAAGATCCTGCGGTCCCTGGAAGGCTCTGATGATGCTGTGCTGCTTCAGA |
| | | GGCGGCTGGACAACATGAACTTCAAGTGGAGCGAGCTGCGGAAGAAGTCCCTGAACATCAGAAGC |
| | | CACCTGGAAGCCTCCAGCGACCAGTGGAAAAGACTGCACCTGTCTCTCCAAGAGCTGCTCGTGTG |
| | | GCTGCAGCTGAAGGATGACGAGCTGAGTAGACAGGCCCCTATCGGCGGAGATTTTCCCGCCGTGCA |
| | | GAAACAGAACGACGTGCACCGGGCCTTCAAGAGAGAGCTGAAAACAAAAGAACCCGTCATCATG |
| | | AGCACCCTGGAAACCGTGCGGATCTTTCTGACCGAGCAGCCTCTGGAAGGACTGGAAAAGCTGTA |
| | | CCAAGAGCCTAGAGAGCTGCCTCCTGAAGAAAGGGCCCAGAACGTGACACGGCTGCTTAGAAAGC |
| | | AGGCCGAGGAAGTGAACACCGAATGGGAGAAGCTGAACCTCCACTCCGCCGACTGGCAGCGGAA |
| | | GATCGATGAGACACTGGAACGGCTGCGGGAACTGCAAGAAGCCACAGACGAGCTGGACCTGAAA |
| | | CTGCGGCAGGCTGAAGTGATCAAAGGCTCCTGGCAGCCAGTGGGCGATCTGCTGATCGATAGCCTG |
| | | CAGGACCATCTGGAAAAAGTCAAGGCCCTGCGGGGAGAGATCGCCCCTCTCAAAGAAAACGTGTC |
| | | CCACGTGAACGATCTGGCCAGGCAGCTGACAACACTGGGCATCCAGCTGAGCCCTTACAACCTGTC |
| | | TACCTTGGAGGACCTGAATACCCGGTGGAAGCTGCTCCAAGTGGCCGTCGAGGATAGAGTGCGGC |
| | | AGCTGCATGAGGCTCACAGAGATTTTGGACCCGCCAGCCAGCACTTCCTGAGCACATCTGTTCAAG |
| | | GCCCCTGGGAGAGAGCTATCAGCCCTAACAAGGTGCCCTACTACATCAACCACGAGACACAGACC |
| | | ACCTGTTGGGATCACCCCAAGATGACCGAACTGTATCAGTCCCTGGCCGACCTGAACAACGTGCGG |
| | | TTTAGCGCCTACCGGACCGCCATGAAGCTGCGCAGACTGCAGAAAGCTCTGTGCCTGGACCTGCTG |
| | | TCTCTGAGCGCTGCTTGTGATGCCCTGGACCAGCATAACCTCAAGCAGAACGACCAGCCTATGGAC |
| | | ATCCTGCAGATCATCAACTGCCTGACCACCATCTACGACCGGCTCGAACAAGAGCACAACAACCTG |
| | | GTCAACGTGCCCCTGTGCGTGGACATGTGCCTGAATTGGCTGCTGAACGTGTACGACACCGGCAGA |
| | | ACCGGCAGGATCAGAGTGCTGAGCTTCAAGACCGGCATCATCTCCCTGTGCAAGGCCCACCTTGAG |
| | | GATAAGTACCGCTACCTGTTTAAGCAGGTCGCCAGCAGCACCGGCTTTTGCGACCAAAGAAGGCTG |
| | | GGACTGCTGCTCCACGACAGCATTCAGATCCCTAGACAGCTGGGCGAAGTGGCCTCTTTCGGCGGC |
| | | TCTAATATCGAGCCTAGCGTGCGGAGCTGCTTCCAGTTCGCCAACAACAAGCCCGAGATTGAGGCC |
| | | GCACTGTTCCTGGACTGGATGAGACTGGAACCCCAGAGCATGGTCTGGCTGCCTGTGCTGCATAGA |
| | | GTGGCCGCAGCCGAAACAGCCAAGCACCAGGCCAAGTGCAACATCTGCAAAGAGTGCCCCATCAT |
| | | CGGCTTCCGGTACAGATCCCTGAAGCACTTCAACTACGATATCTGCCAGTCCTGCTTCTTCAGCGGC |
| | | AGAGTGGCCAAGGGCCACAAGATGCACTACCCCATGGTGGAATACTGCACCCCTACCACCTCTGGC |
| | | GAGGACGTGCGGGATTTTGCCAAGGTGCTCAAGAACAAGTTCCGGACCAAGCGCTACTTCGCTAA |
| | | GCACCCCAGAATGGGCTACCTGCCAGTGCAGACAGTGCTTGAGGGCGACAACATGGAAACCCCTG |
| | | TGACTCTGATCAACTTCTGGCCCGTGGATAGCGCCCCTGCTAGTTCTCCTCAGCTGTCTCACGACGA |
| | | TACCCACAGCAGAATCGAGCACTACGCCTCCAGACTGGCCGAGATGGAAAACAGCAACGGCAGCT |
| | | ACCTGAATGACAGCATCAGCCCCAACGAGAGCATCGACGACGAACATCTGCTCATTCAGCACTACT |
| | | GCCAGAGCCTGAATCAGGACAGCCCTCTGTCTCAGCCTAGAAGCCCTGCACAGATCCTGATCAGCC |
| | | TGGAAAGCGAGGAACGCGGCGAGCTGGAAAGAATCCTGGCAGACCTGGAAGAGGAAAACCGGAA |
| | | CCTGCAGGCCGAATACGACAGACTCAAGCAGCAGCACGAGCACAAGGGACTGTCTCCACTGCCTT |
| | | CTCCACCTGAAATGATGCCCACCTCTCCACAGAGCCCCAGAGATGCCGAGCTGATTGCCGAAGCCA |
| | | AACTGCTGAGGCAGCACAAAGGCAGACTCGAAGCCAGAATGCAGATTCTCGAAGATCACAACAAG |
| | | CAGCTCGAATCCCAGCTGCACAGACTTAGGCAGCTGCTGGAACAGCCTCAGGCAGAGGCCAAAGT |
| | | GAATGGCACCACCGTGTCTAGCCCTAGCACAAGCCTGCAGAGATCCGACAGCAGCCAGCCAATGCT |
| | | TCTGAGAGTCGTGGGCTCCCAGACCAGCGATTCTATGGGCGAAGAGGACCTGCTCAGCCCTCCTCA |
| | | GGATACAAGCACCGGCCTGGAAGAAGTGATGGAACAACTGAACAACAGCTTCCCCAGCAGCAGAG |
| | | GCAGAAACACCCCTGGCAAGCCCATGCGCGAGGACACCATGTGATAA |
| 35. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 4-WPRE3- | TGCCCAAGGACTAAAAAAAGGCCATGGAGCCAGAGGGGCGAGGGCAACAGACCTTTCATGGGCA |
| | | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTT |
| | | TGGTGGGAAGAAGTCGAGGACTGCTACGAGCGCGAGGACGTGCAGAAGAAAACCTTCACCAAAT |
| | | GGGTCAACGCCCAGTTCAGCAAGTTCGGCAAGCAGCACATCGAGAACCTGTTCAGCGACCTGCAG |
| | | GATGGCAGAAGGCTGCTGGATCTGCTGGAAGGCCTGACAGGACAGAAGCTGCCCAAAGAGAAGG |
| | | GCAGCACAAGAGTGCACGCCCTGAACAACGTGAACAAGGCCCTGAGAGTGCTGCAGAACAACAA |
| | | CGTGGACCTGGTCAACATCGGCAGCACCGACATCGTGGACGGCAACCACAAACTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATTCTGCTGAGCTGGGTCCGACAGAGCACCCGGAATTACCCTCAA |
| | | GTGAACGTGATCAACTTCACCACCTCTTGGAGCGACGGACTGGCCCTGAATGCCCTGATCCACAGC |
| | | CACAGACCTGACCTGTTCGACTGGAACAGCGTCGTGTGTCAGCAGAGCGCCACACAGAGGCTGGA |
| | | ACACGCCTTCAATATCGCCAGATACCAGCTGGGCATCGAGAAACTGCTGGACCCCGAGGATGTGGA |
| | | CACCACCTATCCTGACAAGAAATCCATCCTCATGTACATCACCAGCCTGTTCCAGGTGCTGCCCCAG |
| | | CAGGTTTCCATCGAGGCCATTCAAGAGGTCGAGATGCTGCCCAGACCTCCTAAAGTGACCAAAGAG |
| | | GAACACTTCCAGCTGCACCACCAGATGCACTACTCTCAGCAGATCACCGTGTCTCTGGCCCAGGGC |
| | | TACGAGAGAACAAGCAGCCCCAAGCCTCGGTTCAAGAGCTACGCCTATACACAGGCCGCCTACGTG |
| | | ACCACCAGCGATCCTACAAGAAGCCCATTTCCTAGCCAGCATCTGGAAGCCCCTGAGGACAAGAGC |
| | | TTTGGCAGCAGCCTGATGGAAAGCGAAGTGAACCTGGACCGCTACCAGACAGCCCTGGAAGAGGT |
| | | TCTGAGCTGGCTGCTGTCTGCCGAGGATACACTGCAGGCTCAGGGCGAGATCAGCAACGACGTGG |
| | | AAGTGGTCAAGGACCAGTTTCACACCCACGAGGGCTACATGATGGACCTGACAGCCCACCAGGGC |
| | | AGAGTGGGCAATATTCTGCAGCTGGGCTCCAAGCTGATCGGCACAGGCAAGCTGAGCGAGGACGA |
| | | AGAGACAGAGGTGCAAGAGCAGATGAACCTGCTGAACAGCAGATGGGAGTGTCTGAGAGTGGCC |
| | | AGCATGGAAAAGCAGAGCAACCTGCACCGGGTGCTGATGGATCTCCAGAACCAGAAGCTGAAAGA |
| | | GCTGAACGACTGGCTGACCAAGACCGAGGAACGGACCCGGAAGATGGAAGAGGAACCTCTGGGA |
| | | CCCGACCTGGAAGATCTGAAAAGACAGGTGCAGCAGCATAAGGTGCTGCAAGAGGACCTCGAACA |
| | | AGAGCAAGTGCGCGTGAACAGCCTGACACACATGGTGGTGGTCGTGGATGAGAGCAGCGGAGATC |
| | | ATGCCACAGCCGCTCTGGAAGAACAGCTGAAGGTGCTGGGAGACAGATGGGCCAATATCTGCCGG |
| | | TGGACCGAGGATAGATGGGTGCTGCTCCAGGACATCCTGCTGAAGTGGCAGCGGCTGACAGAGGA |
| | | ACAGTGCCTGTTTAGCGCCTGGCTGTCCGAGAAAGAGGACGCCGTCAACAAGATCCACACCACCG |
| | | GCTTCAAGGATCAGAATGAGATGCTGAGCAGCCTGCAGAAACTGGCCGTGCTGAAGGCTGACCTG |
| | | GAAAAGAAAAAGCAGTCCATGGGCAAGCTGTACTCCCTGAAGCAGGACCTGCTGAGCACACTGAA |
| | | GAACAAGTCTGTGACCCAGAAAACCGAGGCCTGGCTGGACAACTTCGCCCGGTGTTGGGATAACC |
| | | TGGTGCAGAAGCTGGAAAAGTCCACCGCTCAGATCTCTCAGGCCGTGACCACAACACAGCCTTCT |
| | | CTGACCCAGACCACCGTGATGGAAACAGTGACCACAGTGACAACCCGCGAGCAGATCCTGGTCAA |
| | | GCACGCCCAAGAAGAACTGCCTCCTCCACCTCCTCAGAAGAAACGGCAGATCACAGTGGACAGCG |
| | | AGATCCGGAAGAGACTGGACGTGGACATCACCGAGCTGCACAGCTGGATCACCAGATCCGAAGCC |
| | | GTGCTGCAGTCCCCTGAGTTCGCCATCTTCAGAAAAGAGGGCAACTTCTCCGACCTGAAAGAGAA |
| | | AGTCAACGCCATCGAGAGAGAGAAGGCCGAGAAGTTCCGGAAGCTGCAGGACGCCTCTAGATCTG |
| | | CCCAGGCTCTGGTGGAACAGATGGTCAACGAAGGCGTGAACGCCGACAGCATCAAGCAGGCCTCA |
| | | GAGCAGCTGAACTCCCGGTGGATCGAGTTCTGTCAGCTCCTGAGCGAGAGACTGAACTGGCTGGA |
| | | ATACCAGAACAATATCATTGCCTTCTACAACCAGCTCCAGCAGCTCGAACAGATGACCACCACAGC |
| | | CGAGAATTGGCTGAAGATCCAGCCTACCACACCTAGCGAGCCCACCGCCATTAAGAGCCAGCTGAA |
| | | AATCTGCAAGGACGAAGTGAATCGGCTGAGCGATCTGCAGCCCCAGATCGAAAGACTGAAAATCC |
| | | AGTCTATCGCCCTCAAAGAGAAAGGACAGGGCCCCATGTTCCTGGACGCCGATTTTGTGGCCTTTA |
| | | CCAACCACTTCAAACAGGTGTTCTCCGACGTGCAGGCCCGGGAAAAAGAGCTGCAGACCATCTTC |
| | | GACACCCTGCCTCCAATGAGATACCAAGAGACAATGAGCGCCATCCGGACCTGGGTGCAGCAAAG |
| | | CGAGACAAAGCTGAGCATCCCACAGCTGAGCGTGACCGACTACGAGATCATGGAACAGAGACTGG |
| | | GCGAACTGCAGGCCCTCCAGTCTAGCCTGCAAGAACAGCAGTCCGGCCTGTACTACCTGAGCACA |
| | | ACCGTGAAAGAGATGAGCAAGAAGGCCCCTAGCGAGATCTCCCGGAAGTACCAGAGCGAGTTCGA |
| | | AGAGATCGAAGGCCGGTGGAAGAAGCTGTCTAGCCAGCTGGTTGAGCACTGCCAGAAACTCGAAG |
| | | AACAAATGAACAAGCTGCGCAAGATCCAGAATCACATCCAGACGCTGAAGAAATGGATGGCCGAG |
| | | GTGGACGTGTTCCTGAAAGAAGAGTGGCCCGCTCTGGGCGACTCCGAGATTCTGAAGAAACAGCT |
| | | CAAACAGTGCCGGCTGCTGGTGTCCGATATCCAGACAATCCAGCCAAGCCTGAACTCCGTGAATGA |
| | | AGGCGGCCAGAAGATCAAGAACGAGGCCGAGCCTGAGTTTGCCAGCAGACTGGAAACCGAACTG |
| | | AAAGAACTCAACACCCAGTGGGACCACATGTGCCAGCAAGTGTACGCCCGGAAAGAGGCCCTGAA |
| | | AGGCGGACTCGAAAAGACTGTGTCTCTGCAGAAAGACCTGTCTGAGATGCACGAGTGGATGACCC |
| | | AGGCCGAAGAGGAATACCTGGAACGGGACTTCGAGTACAAGACCCCTGATGAGCTGCAAAAAGCC |
| | | GTCGAGGAAATGAAGCGGGCCAAAGAAGAGGCCCAGCAGAAAGAAGCCAAAGTCAAGCTGCTGA |
| | | CCGAGTCCGTGAATAGCGTTATCGCTCAGGCCCCTCCTGTGGCTCAAGAGGCTCTCAAGAAAGAAC |
| | | TGGAAACTCTGACCACCAACTACCAGTGGCTGTGCACCAGACTGAACGGCAAGTGCAAGACACTG |
| | | GAAGAAGTGTGGGCCTGCTGGCACGAACTGCTGTCCTATCTGGAAAAGGCCAACAAGTGGCTGAA |
| | | CGAGGTGGAATTCAAGCTGAAAACCACCGAGAACATCCCTGGCGGCGCTGAAGAGATTAGCGAGG |
| | | TGCTGGACAGCCTGGAAAACCTGATGAGACACAGCGAGGACAACCCCAATCAGATCAGAATCCTG |
| | | GCTCAGACCCTGACCGATGGCGGCGTGATGGACGAGCTGATCAACGAGGAACTCGAAACCTTCAA |
| | | CAGCCGGTGGCGGGAACTGCACGAGGAAGCTGTTCGGAGGCAGAAGTTGCTGGAACAGTCTATCC |
| | | AGAGCGCACAAGAAACCGAGAAGTCCCTGCACCTGATCCAAGAGAGCCTGACCTTCATCGACAAG |
| | | CAGCTGGCCGCTTATATCGCCGACAAGGTGGACGCTGCTCAGATGCCCCAAGAGGCACAGAAGATT |
| | | CAGAGCGACCTGACCAGCCACGAGATTAGCCTGGAAGAAATGAAGAAGCACAACCAGGGCAAAG |
| | | AGGCCGCTCAGAGAGTCCTGAGCCAGATCGATGTGGCACAGAAAAAGCTCCAGGATGTGTCCATG |
| | | AAGTTTCGGCTGTTTCAGAAGCCCGCCAACTTCGAGCAGAGACTGCAAGAGTCCAAGATGATCCT |
| | | GGACGAAGTCAAAATGCATCTGCCCGCACTGGAAACAAAGTCCGTGGAACAAGAAGTGGTGCAGT |
| | | CCCAGCTGAACCACTGCGTGAACCTGTACAAGAGCCTGTCCGAAGTGAAGTCTGAGGTGGAAATG |
| | | GTCATCAAGACCGGCAGGCAGATCGTCCAGAAGAAGCAGACCGAGAATCCCAAAGAACTCGACG |
| | | AGAGAGTGACCGCTCTGAAGCTGCACTACAATGAGCTGGGCGCCAAAGTGACCGAGCGGAAGCA |
| | | ACAGCTTGAGAAGTGCCTGAAACTGTCTCGGAAGATGCGGAAAGAAATGAACGTGCTGACAGAGT |
| | | GGCTGGCCGCCACCGATATGGAACTGACTAAGAGAAGCGCCGTGGAAGGCATGCCCAGCAACCTG |
| | | GATAGTGAAGTGGCCTGGGGCAAAGCCACTCAGAAAGAGATTGAGAAGCAGAAGGTCCACCTGA |
| | | AGTCCATCACCGAAGTGGGCGAAGCCCTGAAAACCGTGCTGGGAAAGAAAGAGACACTGGTCGA |
| | | GGACAAGCTGTCCCTGCTGAATTCCAACTGGATCGCCGTGACCTCCAGAGCTGAGGAATGGCTGAA |
| | | TCTGCTGCTTGAGTACCAGAAACACATGGAAACGTTCGACCAGAACGTCGACCACATCACAAAGT |
| | | GGATCATCCAGGCTGATACCCTGCTGGACGAGTCCGAGAAGAAGAAACCCCAACAAAAAGAAGAT |
| | | GTGCTGAAGCGGCTGAAAGCCGAGCTGAATGACATCCGGCCTAAGGTGGACAGCACCAGAGATCA |
| | | GGCAGCCAACCTGATGGCCAACAGAGGCGACCACTGTCGGAAGCTCGTGGAACCTCAGATCAGCG |
| | | AGCTTAACCACAGATTTGCCGCCATCAGCCACCGGATCAAGACAGGCAAGGCCAGCATTCCTCTCA |
| | | AAGAGCTTGAGCAGTTCAACAGCGACATCCAAAAGCTGCTCGAACCCCTGGAAGCCGAGATCCAA |
| | | CAGGGCGTCAACCTCAAAGAAGAAGATTTCAACAAGGACATGAACGAGGACAATGAGGGCACCG |
| | | TCAAAGAACTGCTGCAGCGGGGCGATAATCTGCAGCAGCGGATTACCGATGAGCGCAAGCGGGAA |
| | | GAGATCAAGATTAAGCAGCAGCTGCTGCAGACCAAGCACAACGCCCTGAAGGACCTGCGGAGCCA |
| | | GAGAAGAAAGAAGGCCCTGGAAATCAGCCACCAGTGGTATCAGTACAAGCGGCAGGCCGACGAC |
| | | CTGCTGAAGTGCCTGGATGACATCGAGAAGAAGCTGGCCTCTCTGCCCGAGCCTAGGGACGAGAG |
| | | AAAGATCAAAGAGATCGACCGCGAGCTGCAGAAAAAGAAAGAGGAACTGAACGCCGTCCGCAGA |
| | | CAGGCCGAAGGACTTTCTGAAGATGGCGCCGCTATGGCCGTGGAACCCACACAGATTCAGCTGAG |
| | | CAAGCGGTGGCGGGAAATCGAGAGCAAGTTCGCCCAGTTCCGGCGGCTGAATTTCGCCCAGATCC |
| | | ACACCGTGCGGGAAGAGACAATGATGGTCATGACAGAGGACATGCCCCTTGAGATCAGCTACGTGC |
| | | CCAGCACCTACCTGACCGAGATCACCCATGTGTCTCAGGCCCTGCTGGAAGTGGAACAGCTGCTGA |
| | | ATGCCCCTGACCTGTGCGCCAAGGACTTCGAGGACCTGTTCAAGCAAGAGGAAAGCCTGAAGAAC |
| | | ATCAAGGACAGCCTGCAGCAGAGCAGCGGCCGGATCGATATCATCCACAGCAAGAAAACCGCCGC |
| | | ACTGCAGAGCGCCACACCTGTGGAAAGAGTGAAGCTGCAAGAGGCCCTGAGCCAGCTGGATTTCC |
| | | AGTGGGAGAAAGTGAACAAGATGTACAAGGACCGGCAGGGCAGATTCGACCGCAGCGTTGAAAA |
| | | GTGGCGGCGGTTCCACTACGACATCAAGATCTTCAACCAGTGGCTGACCGAGGCCGAGCAGTTCCT |
| | | GAGAAAGACACAGATCCCCGAGAACTGGGAGCACGCCAAGTACAAGTGGTATCTGAAAGAGCTGC |
| | | AGGACGGCATCGGCCAGAGACAGACAGTCGTGCGGACACTGAATGCCACCGGCGAGGAAATCATC |
| | | CAGCAGTCCAGCAAGACCGACGCCAGCATCCTGCAAGAGAAGCTGGGCAGCCTGAACCTGCGGTG |
| | | GCAAGAAGTGTGCAAGCAGCTGAGCGACCGGAAGAAGCGGCTGGAAGAACAGAAGAATATCCTG |
| | | AGCGAGTTCCAGCGGGACCTGAACGAGTTCGTGCTGTGGCTCGAAGAGGCCGACAATATCGCCAG |
| | | CATTCCCCTGGAACCTGGCAAAGAGCAGCAGCTGAAAGAAAAGCTGGAACAAGTGAAACTGCTG |
| | | GTCGAGGAACTGCCCCTGAGACAGGGAATCCTGAAACAGCTGAACGAGACAGGCGGCCCTGTGCT |
| | | GGTGTCTGCCCCTATTTCTCCCGAGGAACAGGACAAGCTGGAAAACAAGCTGAAGCAGACCAACC |
| | | TGCAGTGGATCAAGGTGTCCAGAGCACTGCCTGAGAAGCAGGGCGAGATCGAGGCCCAGATCAAG |
| | | GACCTGGGACAGCTCGAAAAGAAGCTTGAGGACCTCGAAGAACAGCTCAACCATCTGCTGCTTTG |
| | | GCTGAGCCCCATCCGGAACCAGCTGGAAATCTACAACCAGCCTAATCAAGAGGGCCCCTTCGACGT |
| | | GAAAGAAACCGAGATTGCCGTGCAGGCCAAGCAGCCCGATGTGGAAGAGATCCTGTCTAAGGGCC |
| | | AGCACCTGTACAAAGAGAAGCCCGCCACACAGCCCGTGAAGCGGAAACTGGAAGATCTGAGCAG |
| | | CGAGTGGAAGGCCGTGAACCGGCTGCTGCAAGAACTGAGAGCCAAACAGCCTGATCTGGCCCCTG |
| | | GCCTGACAACAATTGGCGCCTCTCCTACACAGACCGTGACACTGGTCACACAGCCTGTGGTCACCA |
| | | AAGAGACAGCCATCAGCAAACTGGAAATGCCCAGCAGTCTGATGCTCGAAGTGCCCGCACTGGCC |
| | | GACTTCAATAGAGCCTGGACCGAGCTGACCGACTGGCTCAGTCTGCTGGACCAAGTCATCAAGTCC |
| | | CAGAGAGTGATGGTCGGAGATCTTGAGGACATCAACGAGATGATCATTAAGCAGAAAGCCACCATG |
| | | CAGGACCTTGAGCAGAGAAGGCCCCAGCTTGAAGAACTGATCACCGCCGCTCAGAACCTGAAAAA |
| | | CAAGACCAGCAATCAAGAAGCCCGGACCATCATCACCGACCGGATCGAGAGAATCCAGAACCAGT |
| | | GGGACGAAGTGCAAGAGCATCTGCAGAACAGACGGCAGCAGCTCAATGAGATGCTGAAGGACAG |
| | | CACACAGTGGCTGGAAGCCAAAGAAGAAGCCGAGCAGGTTCTCGGACAGGCCAGAGCTAAGCTG |
| | | GAATCTTGGAAAGAGGGACCCTACACCGTCGACGCCATCCAGAAGAAGATCACCGAGACAAAGCA |
| | | GCTGGCCAAGGATCTGAGACAGTGGCAGACCAATGTGGACGTGGCCAACGACCTGGCTCTGAAGC |
| | | TGCTGAGAGACTACAGCGCCGACGACACCAGAAAGGTGCACATGATCACCGAAAACATCAACGCC |
| | | TCTTGGCGGAGCATCCACAAGCGGGTGTCAGAGAGAGAAGCCGCTCTGGAAGAAACACATAGACT |
| | | GCTCCAGCAGTTCCCACTCGACCTGGAAAAGTTCCTGGCCTGGCTGACAGAAGCCGAAACCACCG |
| | | CTAACGTGCTCCAGGATGCCACCAGAAAAGAGCGGCTGTTGGAGGACAGCAAGGGCGTCAAAGA |
| | | ACTCATGAAGCAGTGGCAGGATCTGCAAGGGGAGATTGAAGCCCACACCGACGTGTACCACAACC |
| | | TGGACGAGAACAGCCAGAAGATCCTGCGGTCCCTGGAAGGCTCTGATGATGCTGTGCTGCTTCAGA |
| | | GGCGGCTGGACAACATGAACTTCAAGTGGAGCGAGCTGCGGAAGAAGTCCCTGAACATCAGAAGC |
| | | CACCTGGAAGCCTCCAGCGACCAGTGGAAAAGACTGCACCTGTCTCTCCAAGAGCTGCTCGTGTG |
| | | GCTGCAGCTGAAGGATGACGAGCTGAGTAGACAGGCCCCTATCGGCGGAGATTTTCCCGCCGTGCA |
| | | GAAACAGAACGACGTGCACCGGGCCTTCAAGAGAGAGCTGAAAACAAAAGAACCCGTCATCATG |
| | | AGCACCCTGGAAACCGTGCGGATCTTTCTGACCGAGCAGCCTCTGGAAGGACTGGAAAAGCTGTA |
| | | CCAAGAGCCTAGAGAGCTGCCTCCTGAAGAAAGGGCCCAGAACGTGACACGGCTGCTTAGAAAGC |
| | | AGGCCGAGGAAGTGAACACCGAATGGGAGAAGCTGAACCTCCACTCCGCCGACTGGCAGCGGAA |
| | | GATCGATGAGACACTGGAACGGCTGCGGGAACTGCAAGAAGCCACAGACGAGCTGGACCTGAAA |
| | | CTGCGGCAGGCTGAAGTGATCAAAGGCTCCTGGCAGCCAGTGGGCGATCTGCTGATCGATAGCCTG |
| | | CAGGACCATCTGGAAAAAGTCAAGGCCCTGCGGGGAGAGATCGCCCCTCTCAAAGAAAACGTGTC |
| | | CCACGTGAACGATCTGGCCAGGCAGCTGACAACACTGGGCATCCAGCTGAGCCCTTACAACCTGTC |
| | | TACCTTGGAGGACCTGAATACCCGGTGGAAGCTGCTCCAAGTGGCCGTCGAGGATAGAGTGCGGC |
| | | AGCTGCATGAGGCTCACAGAGATTTTGGACCCGCCAGCCAGCACTTCCTGAGCACATCTGTTCAAG |
| | | GCCCCTGGGAGAGAGCTATCAGCCCTAACAAGGTGCCCTACTACATCAACCACGAGACACAGACC |
| | | ACCTGTTGGGATCACCCCAAGATGACCGAACTGTATCAGTCCCTGGCCGACCTGAACAACGTGCGG |
| | | TTTAGCGCCTACCGGACCGCCATGAAGCTGCGCAGACTGCAGAAAGCTCTGTGCCTGGACCTGCTG |
| | | TCTCTGAGCGCTGCTTGTGATGCCCTGGACCAGCATAACCTCAAGCAGAACGACCAGCCTATGGAC |
| | | ATCCTGCAGATCATCAACTGCCTGACCACCATCTACGACCGGCTCGAACAAGAGCACAACAACCTG |
| | | GTCAACGTGCCCCTGTGCGTGGACATGTGCCTGAATTGGCTGCTGAACGTGTACGACACCGGCAGA |
| | | ACCGGCAGGATCAGAGTGCTGAGCTTCAAGACCGGCATCATCTCCCTGTGCAAGGCCCACCTTGAG |
| | | GATAAGTACCGCTACCTGTTTAAGCAGGTCGCCAGCAGCACCGGCTTTTGCGACCAAAGAAGGCTG |
| | | GGACTGCTGCTCCACGACAGCATTCAGATCCCTAGACAGCTGGGCGAAGTGGCCTCTTTCGGCGGC |
| | | TCTAATATCGAGCCTAGCGTGCGGAGCTGCTTCCAGTTCGCCAACAACAAGCCCGAGATTGAGGCC |
| | | GCACTGTTCCTGGACTGGATGAGACTGGAACCCCAGAGCATGGTCTGGCTGCCTGTGCTGCATAGA |
| | | GTGGCCGCAGCCGAAACAGCCAAGCACCAGGCCAAGTGCAACATCTGCAAAGAGTGCCCCATCAT |
| | | CGGCTTCCGGTACAGATCCCTGAAGCACTTCAACTACGATATCTGCCAGTCCTGCTTCTTCAGCGGC |
| | | AGAGTGGCCAAGGGCCACAAGATGCACTACCCCATGGTGGAATACTGCACCCCTACCACCTCTGGC |
| | | GAGGACGTGCGGGATTTTGCCAAGGTGCTCAAGAACAAGTTCCGGACCAAGCGCTACTTCGCTAA |
| | | GCACCCCAGAATGGGCTACCTGCCAGTGCAGACAGTGCTTGAGGGCGACAACATGGAAACCCCTG |
| | | TGACTCTGATCAACTTCTGGCCCGTGGATAGCGCCCCTGCTAGTTCTCCTCAGCTGTCTCACGACGA |
| | | TACCCACAGCAGAATCGAGCACTACGCCTCCAGACTGGCCGAGATGGAAAACAGCAACGGCAGCT |
| | | ACCTGAATGACAGCATCAGCCCCAACGAGAGCATCGACGACGAACATCTGCTCATTCAGCACTACT |
| | | GCCAGAGCCTGAATCAGGACAGCCCTCTGTCTCAGCCTAGAAGCCCTGCACAGATCCTGATCAGCC |
| | | TGGAAAGCGAGGAACGCGGCGAGCTGGAAAGAATCCTGGCAGACCTGGAAGAGGAAAACCGGAA |
| | | CCTGCAGGCCGAATACGACAGACTCAAGCAGCAGCACGAGCACAAGGGACTGTCTCCACTGCCTT |
| | | CTCCACCTGAAATGATGCCCACCTCTCCACAGAGCCCCAGAGATGCCGAGCTGATTGCCGAAGCCA |
| | | AACTGCTGAGGCAGCACAAAGGCAGACTCGAAGCCAGAATGCAGATTCTCGAAGATCACAACAAG |
| | | CAGCTCGAATCCCAGCTGCACAGACTTAGGCAGCTGCTGGAACAGCCTCAGGCAGAGGCCAAAGT |
| | | GAATGGCACCACCGTGTCTAGCCCTAGCACAAGCCTGCAGAGATCCGACAGCAGCCAGCCAATGCT |
| | | TCTGAGAGTCGTGGGCTCCCAGACCAGCGATTCTATGGGCGAAGAGGACCTGCTCAGCCCTCCTCA |
| | | GGATACAAGCACCGGCCTGGAAGAAGTGATGGAACAACTGAACAACAGCTTCCCCAGCAGCAGAG |
| | | GCAGAAACACCCCTGGCAAGCCCATGCGCGAGGACACCATGTGATAACTCGAGAATCAACCTCTG |
| | | GATTACAAAATTTGTGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCTTTCCGGGACTTT |
| | | CGCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGG |
| 36. | MHCK7- | CTAGAAGCTGCATGTCTAAGCTAGACCCTTCAGATTAAAAATAACTGAGGTAAGGGCCTGGGTAGG |
| | hBGi-DMD- | GGAGGTGGTGTGAGACGCTCCTGTCTCTCCTCTATCTGCCCATCGGCCCTTTGGGGAGGAGGAATG |
| | 4-WPRE3- | TGCCCAAGGACTAAAAAAAGGCCATGGAGCCAGAGGGGCGAGGGCAACAGACCTTTCATGGGCA |
| | Poly A | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGCTT |
| | | TGGTGGGAAGAAGTCGAGGACTGCTACGAGCGCGAGGACGTGCAGAAGAAAACCTTCACCAAAT |
| | | GGGTCAACGCCCAGTTCAGCAAGTTCGGCAAGCAGCACATCGAGAACCTGTTCAGCGACCTGCAG |
| | | GATGGCAGAAGGCTGCTGGATCTGCTGGAAGGCCTGACAGGACAGAAGCTGCCCAAAGAGAAGG |
| | | GCAGCACAAGAGTGCACGCCCTGAACAACGTGAACAAGGCCCTGAGAGTGCTGCAGAACAACAA |
| | | CGTGGACCTGGTCAACATCGGCAGCACCGACATCGTGGACGGCAACCACAAACTGACCCTGGGCC |
| | | TGATCTGGAACATCATCCTGCACTGGCAAGTGAAGAACGTGATGAAGAACATCATGGCCGGCCTGC |
| | | AGCAGACCAACAGCGAGAAGATTCTGCTGAGCTGGGTCCGACAGAGCACCCGGAATTACCCTCAA |
| | | GTGAACGTGATCAACTTCACCACCTCTTGGAGCGACGGACTGGCCCTGAATGCCCTGATCCACAGC |
| | | CACAGACCTGACCTGTTCGACTGGAACAGCGTCGTGTGTCAGCAGAGCGCCACACAGAGGCTGGA |
| | | ACACGCCTTCAATATCGCCAGATACCAGCTGGGCATCGAGAAACTGCTGGACCCCGAGGATGTGGA |
| | | CACCACCTATCCTGACAAGAAATCCATCCTCATGTACATCACCAGCCTGTTCCAGGTGCTGCCCCAG |
| | | CAGGTTTCCATCGAGGCCATTCAAGAGGTCGAGATGCTGCCCAGACCTCCTAAAGTGACCAAAGAG |
| | | GAACACTTCCAGCTGCACCACCAGATGCACTACTCTCAGCAGATCACCGTGTCTCTGGCCCAGGGC |
| | | TACGAGAGAACAAGCAGCCCCAAGCCTCGGTTCAAGAGCTACGCCTATACACAGGCCGCCTACGTG |
| | | ACCACCAGCGATCCTACAAGAAGCCCATTTCCTAGCCAGCATCTGGAAGCCCCTGAGGACAAGAGC |
| | | TTTGGCAGCAGCCTGATGGAAAGCGAAGTGAACCTGGACCGCTACCAGACAGCCCTGGAAGAGGT |
| | | TCTGAGCTGGCTGCTGTCTGCCGAGGATACACTGCAGGCTCAGGGCGAGATCAGCAACGACGTGG |
| | | AAGTGGTCAAGGACCAGTTTCACACCCACGAGGGCTACATGATGGACCTGACAGCCCACCAGGGC |
| | | AGAGTGGGCAATATTCTGCAGCTGGGCTCCAAGCTGATCGGCACAGGCAAGCTGAGCGAGGACGA |
| | | AGAGACAGAGGTGCAAGAGCAGATGAACCTGCTGAACAGCAGATGGGAGTGTCTGAGAGTGGCC |
| | | AGCATGGAAAAGCAGAGCAACCTGCACCGGGTGCTGATGGATCTCCAGAACCAGAAGCTGAAAGA |
| | | GCTGAACGACTGGCTGACCAAGACCGAGGAACGGACCCGGAAGATGGAAGAGGAACCTCTGGGA |
| | | CCCGACCTGGAAGATCTGAAAAGACAGGTGCAGCAGCATAAGGTGCTGCAAGAGGACCTCGAACA |
| | | AGAGCAAGTGCGCGTGAACAGCCTGACACACATGGTGGTGGTCGTGGATGAGAGCAGCGGAGATC |
| | | ATGCCACAGCCGCTCTGGAAGAACAGCTGAAGGTGCTGGGAGACAGATGGGCCAATATCTGCCGG |
| | | TGGACCGAGGATAGATGGGTGCTGCTCCAGGACATCCTGCTGAAGTGGCAGCGGCTGACAGAGGA |
| | | ACAGTGCCTGTTTAGCGCCTGGCTGTCCGAGAAAGAGGACGCCGTCAACAAGATCCACACCACCG |
| | | GCTTCAAGGATCAGAATGAGATGCTGAGCAGCCTGCAGAAACTGGCCGTGCTGAAGGCTGACCTG |
| | | GAAAAGAAAAAGCAGTCCATGGGCAAGCTGTACTCCCTGAAGCAGGACCTGCTGAGCACACTGAA |
| | | GAACAAGTCTGTGACCCAGAAAACCGAGGCCTGGCTGGACAACTTCGCCCGGTGTTGGGATAACC |
| | | TGGTGCAGAAGCTGGAAAAGTCCACCGCTCAGATCTCTCAGGCCGTGACCACAACACAGCCTTCT |
| | | CTGACCCAGACCACCGTGATGGAAACAGTGACCACAGTGACAACCCGCGAGCAGATCCTGGTCAA |
| | | GCACGCCCAAGAAGAACTGCCTCCTCCACCTCCTCAGAAGAAACGGCAGATCACAGTGGACAGCG |
| | | AGATCCGGAAGAGACTGGACGTGGACATCACCGAGCTGCACAGCTGGATCACCAGATCCGAAGCC |
| | | GTGCTGCAGTCCCCTGAGTTCGCCATCTTCAGAAAAGAGGGCAACTTCTCCGACCTGAAAGAGAA |
| | | AGTCAACGCCATCGAGAGAGAGAAGGCCGAGAAGTTCCGGAAGCTGCAGGACGCCTCTAGATCTG |
| | | CCCAGGCTCTGGTGGAACAGATGGTCAACGAAGGCGTGAACGCCGACAGCATCAAGCAGGCCTCA |
| | | GAGCAGCTGAACTCCCGGTGGATCGAGTTCTGTCAGCTCCTGAGCGAGAGACTGAACTGGCTGGA |
| | | ATACCAGAACAATATCATTGCCTTCTACAACCAGCTCCAGCAGCTCGAACAGATGACCACCACAGC |
| | | CGAGAATTGGCTGAAGATCCAGCCTACCACACCTAGCGAGCCCACCGCCATTAAGAGCCAGCTGAA |
| | | AATCTGCAAGGACGAAGTGAATCGGCTGAGCGATCTGCAGCCCCAGATCGAAAGACTGAAAATCC |
| | | AGTCTATCGCCCTCAAAGAGAAAGGACAGGGCCCCATGTTCCTGGACGCCGATTTTGTGGCCTTTA |
| | | CCAACCACTTCAAACAGGTGTTCTCCGACGTGCAGGCCCGGGAAAAAGAGCTGCAGACCATCTTC |
| | | GACACCCTGCCTCCAATGAGATACCAAGAGACAATGAGCGCCATCCGGACCTGGGTGCAGCAAAG |
| | | CGAGACAAAGCTGAGCATCCCACAGCTGAGCGTGACCGACTACGAGATCATGGAACAGAGACTGG |
| | | GCGAACTGCAGGCCCTCCAGTCTAGCCTGCAAGAACAGCAGTCCGGCCTGTACTACCTGAGCACA |
| | | ACCGTGAAAGAGATGAGCAAGAAGGCCCCTAGCGAGATCTCCCGGAAGTACCAGAGCGAGTTCGA |
| | | AGAGATCGAAGGCCGGTGGAAGAAGCTGTCTAGCCAGCTGGTTGAGCACTGCCAGAAACTCGAAG |
| | | AACAAATGAACAAGCTGCGCAAGATCCAGAATCACATCCAGACGCTGAAGAAATGGATGGCCGAG |
| | | GTGGACGTGTTCCTGAAAGAAGAGTGGCCCGCTCTGGGCGACTCCGAGATTCTGAAGAAACAGCT |
| | | CAAACAGTGCCGGCTGCTGGTGTCCGATATCCAGACAATCCAGCCAAGCCTGAACTCCGTGAATGA |
| | | AGGCGGCCAGAAGATCAAGAACGAGGCCGAGCCTGAGTTTGCCAGCAGACTGGAAACCGAACTG |
| | | AAAGAACTCAACACCCAGTGGGACCACATGTGCCAGCAAGTGTACGCCCGGAAAGAGGCCCTGAA |
| | | AGGCGGACTCGAAAAGACTGTGTCTCTGCAGAAAGACCTGTCTGAGATGCACGAGTGGATGACCC |
| | | AGGCCGAAGAGGAATACCTGGAACGGGACTTCGAGTACAAGACCCCTGATGAGCTGCAAAAAGCC |
| | | GTCGAGGAAATGAAGCGGGCCAAAGAAGAGGCCCAGCAGAAAGAAGCCAAAGTCAAGCTGCTGA |
| | | CCGAGTCCGTGAATAGCGTTATCGCTCAGGCCCCTCCTGTGGCTCAAGAGGCTCTCAAGAAAGAAC |
| | | TGGAAACTCTGACCACCAACTACCAGTGGCTGTGCACCAGACTGAACGGCAAGTGCAAGACACTG |
| | | GAAGAAGTGTGGGCCTGCTGGCACGAACTGCTGTCCTATCTGGAAAAGGCCAACAAGTGGCTGAA |
| | | CGAGGTGGAATTCAAGCTGAAAACCACCGAGAACATCCCTGGCGGCGCTGAAGAGATTAGCGAGG |
| | | TGCTGGACAGCCTGGAAAACCTGATGAGACACAGCGAGGACAACCCCAATCAGATCAGAATCCTG |
| | | GCTCAGACCCTGACCGATGGCGGCGTGATGGACGAGCTGATCAACGAGGAACTCGAAACCTTCAA |
| | | CAGCCGGTGGCGGGAACTGCACGAGGAAGCTGTTCGGAGGCAGAAGTTGCTGGAACAGTCTATCC |
| | | AGAGCGCACAAGAAACCGAGAAGTCCCTGCACCTGATCCAAGAGAGCCTGACCTTCATCGACAAG |
| | | CAGCTGGCCGCTTATATCGCCGACAAGGTGGACGCTGCTCAGATGCCCCAAGAGGCACAGAAGATT |
| | | CAGAGCGACCTGACCAGCCACGAGATTAGCCTGGAAGAAATGAAGAAGCACAACCAGGGCAAAG |
| | | AGGCCGCTCAGAGAGTCCTGAGCCAGATCGATGTGGCACAGAAAAAGCTCCAGGATGTGTCCATG |
| | | AAGTTTCGGCTGTTTCAGAAGCCCGCCAACTTCGAGCAGAGACTGCAAGAGTCCAAGATGATCCT |
| | | GGACGAAGTCAAAATGCATCTGCCCGCACTGGAAACAAAGTCCGTGGAACAAGAAGTGGTGCAGT |
| | | CCCAGCTGAACCACTGCGTGAACCTGTACAAGAGCCTGTCCGAAGTGAAGTCTGAGGTGGAAATG |
| | | GTCATCAAGACCGGCAGGCAGATCGTCCAGAAGAAGCAGACCGAGAATCCCAAAGAACTCGACG |
| | | AGAGAGTGACCGCTCTGAAGCTGCACTACAATGAGCTGGGCGCCAAAGTGACCGAGCGGAAGCA |
| | | ACAGCTTGAGAAGTGCCTGAAACTGTCTCGGAAGATGCGGAAAGAAATGAACGTGCTGACAGAGT |
| | | GGCTGGCCGCCACCGATATGGAACTGACTAAGAGAAGCGCCGTGGAAGGCATGCCCAGCAACCTG |
| | | GATAGTGAAGTGGCCTGGGGCAAAGCCACTCAGAAAGAGATTGAGAAGCAGAAGGTCCACCTGA |
| | | AGTCCATCACCGAAGTGGGCGAAGCCCTGAAAACCGTGCTGGGAAAGAAAGAGACACTGGTCGA |
| | | GGACAAGCTGTCCCTGCTGAATTCCAACTGGATCGCCGTGACCTCCAGAGCTGAGGAATGGCTGAA |
| | | TCTGCTGCTTGAGTACCAGAAACACATGGAAACGTTCGACCAGAACGTCGACCACATCACAAAGT |
| | | GGATCATCCAGGCTGATACCCTGCTGGACGAGTCCGAGAAGAAGAAACCCCAACAAAAAGAAGAT |
| | | GTGCTGAAGCGGCTGAAAGCCGAGCTGAATGACATCCGGCCTAAGGTGGACAGCACCAGAGATCA |
| | | GGCAGCCAACCTGATGGCCAACAGAGGCGACCACTGTCGGAAGCTCGTGGAACCTCAGATCAGCG |
| | | AGCTTAACCACAGATTTGCCGCCATCAGCCACCGGATCAAGACAGGCAAGGCCAGCATTCCTCTCA |
| | | AAGAGCTTGAGCAGTTCAACAGCGACATCCAAAAGCTGCTCGAACCCCTGGAAGCCGAGATCCAA |
| | | CAGGGCGTCAACCTCAAAGAAGAAGATTTCAACAAGGACATGAACGAGGACAATGAGGGCACCG |
| | | TCAAAGAACTGCTGCAGCGGGGCGATAATCTGCAGCAGCGGATTACCGATGAGCGCAAGCGGGAA |
| | | GAGATCAAGATTAAGCAGCAGCTGCTGCAGACCAAGCACAACGCCCTGAAGGACCTGCGGAGCCA |
| | | GAGAAGAAAGAAGGCCCTGGAAATCAGCCACCAGTGGTATCAGTACAAGCGGCAGGCCGACGAC |
| | | CTGCTGAAGTGCCTGGATGACATCGAGAAGAAGCTGGCCTCTCTGCCCGAGCCTAGGGACGAGAG |
| | | AAAGATCAAAGAGATCGACCGCGAGCTGCAGAAAAAGAAAGAGGAACTGAACGCCGTCCGCAGA |
| | | CAGGCCGAAGGACTTTCTGAAGATGGCGCCGCTATGGCCGTGGAACCCACACAGATTCAGCTGAG |
| | | CAAGCGGTGGCGGGAAATCGAGAGCAAGTTCGCCCAGTTCCGGCGGCTGAATTTCGCCCAGATCC |
| | | ACACCGTGCGGGAAGAGACAATGATGGTCATGACAGAGGACATGCCCCTTGAGATCAGCTACGTGC |
| | | CCAGCACCTACCTGACCGAGATCACCCATGTGTCTCAGGCCCTGCTGGAAGTGGAACAGCTGCTGA |
| | | ATGCCCCTGACCTGTGCGCCAAGGACTTCGAGGACCTGTTCAAGCAAGAGGAAAGCCTGAAGAAC |
| | | ATCAAGGACAGCCTGCAGCAGAGCAGCGGCCGGATCGATATCATCCACAGCAAGAAAACCGCCGC |
| | | ACTGCAGAGCGCCACACCTGTGGAAAGAGTGAAGCTGCAAGAGGCCCTGAGCCAGCTGGATTTCC |
| | | AGTGGGAGAAAGTGAACAAGATGTACAAGGACCGGCAGGGCAGATTCGACCGCAGCGTTGAAAA |
| | | GTGGCGGCGGTTCCACTACGACATCAAGATCTTCAACCAGTGGCTGACCGAGGCCGAGCAGTTCCT |
| | | GAGAAAGACACAGATCCCCGAGAACTGGGAGCACGCCAAGTACAAGTGGTATCTGAAAGAGCTGC |
| | | AGGACGGCATCGGCCAGAGACAGACAGTCGTGCGGACACTGAATGCCACCGGCGAGGAAATCATC |
| | | CAGCAGTCCAGCAAGACCGACGCCAGCATCCTGCAAGAGAAGCTGGGCAGCCTGAACCTGCGGTG |
| | | GCAAGAAGTGTGCAAGCAGCTGAGCGACCGGAAGAAGCGGCTGGAAGAACAGAAGAATATCCTG |
| | | AGCGAGTTCCAGCGGGACCTGAACGAGTTCGTGCTGTGGCTCGAAGAGGCCGACAATATCGCCAG |
| | | CATTCCCCTGGAACCTGGCAAAGAGCAGCAGCTGAAAGAAAAGCTGGAACAAGTGAAACTGCTG |
| | | GTCGAGGAACTGCCCCTGAGACAGGGAATCCTGAAACAGCTGAACGAGACAGGCGGCCCTGTGCT |
| | | GGTGTCTGCCCCTATTTCTCCCGAGGAACAGGACAAGCTGGAAAACAAGCTGAAGCAGACCAACC |
| | | TGCAGTGGATCAAGGTGTCCAGAGCACTGCCTGAGAAGCAGGGCGAGATCGAGGCCCAGATCAAG |
| | | GACCTGGGACAGCTCGAAAAGAAGCTTGAGGACCTCGAAGAACAGCTCAACCATCTGCTGCTTTG |
| | | GCTGAGCCCCATCCGGAACCAGCTGGAAATCTACAACCAGCCTAATCAAGAGGGCCCCTTCGACGT |
| | | GAAAGAAACCGAGATTGCCGTGCAGGCCAAGCAGCCCGATGTGGAAGAGATCCTGTCTAAGGGCC |
| | | AGCACCTGTACAAAGAGAAGCCCGCCACACAGCCCGTGAAGCGGAAACTGGAAGATCTGAGCAG |
| | | CGAGTGGAAGGCCGTGAACCGGCTGCTGCAAGAACTGAGAGCCAAACAGCCTGATCTGGCCCCTG |
| | | GCCTGACAACAATTGGCGCCTCTCCTACACAGACCGTGACACTGGTCACACAGCCTGTGGTCACCA |
| | | AAGAGACAGCCATCAGCAAACTGGAAATGCCCAGCAGTCTGATGCTCGAAGTGCCCGCACTGGCC |
| | | GACTTCAATAGAGCCTGGACCGAGCTGACCGACTGGCTCAGTCTGCTGGACCAAGTCATCAAGTCC |
| | | CAGAGAGTGATGGTCGGAGATCTTGAGGACATCAACGAGATGATCATTAAGCAGAAAGCCACCATG |
| | | CAGGACCTTGAGCAGAGAAGGCCCCAGCTTGAAGAACTGATCACCGCCGCTCAGAACCTGAAAAA |
| | | CAAGACCAGCAATCAAGAAGCCCGGACCATCATCACCGACCGGATCGAGAGAATCCAGAACCAGT |
| | | GGGACGAAGTGCAAGAGCATCTGCAGAACAGACGGCAGCAGCTCAATGAGATGCTGAAGGACAG |
| | | CACACAGTGGCTGGAAGCCAAAGAAGAAGCCGAGCAGGTTCTCGGACAGGCCAGAGCTAAGCTG |
| | | GAATCTTGGAAAGAGGGACCCTACACCGTCGACGCCATCCAGAAGAAGATCACCGAGACAAAGCA |
| | | GCTGGCCAAGGATCTGAGACAGTGGCAGACCAATGTGGACGTGGCCAACGACCTGGCTCTGAAGC |
| | | TGCTGAGAGACTACAGCGCCGACGACACCAGAAAGGTGCACATGATCACCGAAAACATCAACGCC |
| | | TCTTGGCGGAGCATCCACAAGCGGGTGTCAGAGAGAGAAGCCGCTCTGGAAGAAACACATAGACT |
| | | GCTCCAGCAGTTCCCACTCGACCTGGAAAAGTTCCTGGCCTGGCTGACAGAAGCCGAAACCACCG |
| | | CTAACGTGCTCCAGGATGCCACCAGAAAAGAGCGGCTGTTGGAGGACAGCAAGGGCGTCAAAGA |
| | | ACTCATGAAGCAGTGGCAGGATCTGCAAGGGGAGATTGAAGCCCACACCGACGTGTACCACAACC |
| | | TGGACGAGAACAGCCAGAAGATCCTGCGGTCCCTGGAAGGCTCTGATGATGCTGTGCTGCTTCAGA |
| | | GGCGGCTGGACAACATGAACTTCAAGTGGAGCGAGCTGCGGAAGAAGTCCCTGAACATCAGAAGC |
| | | CACCTGGAAGCCTCCAGCGACCAGTGGAAAAGACTGCACCTGTCTCTCCAAGAGCTGCTCGTGTG |
| | | GCTGCAGCTGAAGGATGACGAGCTGAGTAGACAGGCCCCTATCGGCGGAGATTTTCCCGCCGTGCA |
| | | GAAACAGAACGACGTGCACCGGGCCTTCAAGAGAGAGCTGAAAACAAAAGAACCCGTCATCATG |
| | | AGCACCCTGGAAACCGTGCGGATCTTTCTGACCGAGCAGCCTCTGGAAGGACTGGAAAAGCTGTA |
| | | CCAAGAGCCTAGAGAGCTGCCTCCTGAAGAAAGGGCCCAGAACGTGACACGGCTGCTTAGAAAGC |
| | | AGGCCGAGGAAGTGAACACCGAATGGGAGAAGCTGAACCTCCACTCCGCCGACTGGCAGCGGAA |
| | | GATCGATGAGACACTGGAACGGCTGCGGGAACTGCAAGAAGCCACAGACGAGCTGGACCTGAAA |
| | | CTGCGGCAGGCTGAAGTGATCAAAGGCTCCTGGCAGCCAGTGGGCGATCTGCTGATCGATAGCCTG |
| | | CAGGACCATCTGGAAAAAGTCAAGGCCCTGCGGGGAGAGATCGCCCCTCTCAAAGAAAACGTGTC |
| | | CCACGTGAACGATCTGGCCAGGCAGCTGACAACACTGGGCATCCAGCTGAGCCCTTACAACCTGTC |
| | | TACCTTGGAGGACCTGAATACCCGGTGGAAGCTGCTCCAAGTGGCCGTCGAGGATAGAGTGCGGC |
| | | AGCTGCATGAGGCTCACAGAGATTTTGGACCCGCCAGCCAGCACTTCCTGAGCACATCTGTTCAAG |
| | | GCCCCTGGGAGAGAGCTATCAGCCCTAACAAGGTGCCCTACTACATCAACCACGAGACACAGACC |
| | | ACCTGTTGGGATCACCCCAAGATGACCGAACTGTATCAGTCCCTGGCCGACCTGAACAACGTGCGG |
| | | TTTAGCGCCTACCGGACCGCCATGAAGCTGCGCAGACTGCAGAAAGCTCTGTGCCTGGACCTGCTG |
| | | TCTCTGAGCGCTGCTTGTGATGCCCTGGACCAGCATAACCTCAAGCAGAACGACCAGCCTATGGAC |
| | | ATCCTGCAGATCATCAACTGCCTGACCACCATCTACGACCGGCTCGAACAAGAGCACAACAACCTG |
| | | GTCAACGTGCCCCTGTGCGTGGACATGTGCCTGAATTGGCTGCTGAACGTGTACGACACCGGCAGA |
| | | ACCGGCAGGATCAGAGTGCTGAGCTTCAAGACCGGCATCATCTCCCTGTGCAAGGCCCACCTTGAG |
| | | GATAAGTACCGCTACCTGTTTAAGCAGGTCGCCAGCAGCACCGGCTTTTGCGACCAAAGAAGGCTG |
| | | GGACTGCTGCTCCACGACAGCATTCAGATCCCTAGACAGCTGGGCGAAGTGGCCTCTTTCGGCGGC |
| | | TCTAATATCGAGCCTAGCGTGCGGAGCTGCTTCCAGTTCGCCAACAACAAGCCCGAGATTGAGGCC |
| | | GCACTGTTCCTGGACTGGATGAGACTGGAACCCCAGAGCATGGTCTGGCTGCCTGTGCTGCATAGA |
| | | GTGGCCGCAGCCGAAACAGCCAAGCACCAGGCCAAGTGCAACATCTGCAAAGAGTGCCCCATCAT |
| | | CGGCTTCCGGTACAGATCCCTGAAGCACTTCAACTACGATATCTGCCAGTCCTGCTTCTTCAGCGGC |
| | | AGAGTGGCCAAGGGCCACAAGATGCACTACCCCATGGTGGAATACTGCACCCCTACCACCTCTGGC |
| | | GAGGACGTGCGGGATTTTGCCAAGGTGCTCAAGAACAAGTTCCGGACCAAGCGCTACTTCGCTAA |
| | | GCACCCCAGAATGGGCTACCTGCCAGTGCAGACAGTGCTTGAGGGCGACAACATGGAAACCCCTG |
| | | TGACTCTGATCAACTTCTGGCCCGTGGATAGCGCCCCTGCTAGTTCTCCTCAGCTGTCTCACGACGA |
| | | TACCCACAGCAGAATCGAGCACTACGCCTCCAGACTGGCCGAGATGGAAAACAGCAACGGCAGCT |
| | | ACCTGAATGACAGCATCAGCCCCAACGAGAGCATCGACGACGAACATCTGCTCATTCAGCACTACT |
| | | GCCAGAGCCTGAATCAGGACAGCCCTCTGTCTCAGCCTAGAAGCCCTGCACAGATCCTGATCAGCC |
| | | TGGAAAGCGAGGAACGCGGCGAGCTGGAAAGAATCCTGGCAGACCTGGAAGAGGAAAACCGGAA |
| | | CCTGCAGGCCGAATACGACAGACTCAAGCAGCAGCACGAGCACAAGGGACTGTCTCCACTGCCTT |
| | | CTCCACCTGAAATGATGCCCACCTCTCCACAGAGCCCCAGAGATGCCGAGCTGATTGCCGAAGCCA |
| | | AACTGCTGAGGCAGCACAAAGGCAGACTCGAAGCCAGAATGCAGATTCTCGAAGATCACAACAAG |
| | | CAGCTCGAATCCCAGCTGCACAGACTTAGGCAGCTGCTGGAACAGCCTCAGGCAGAGGCCAAAGT |
| | | GAATGGCACCACCGTGTCTAGCCCTAGCACAAGCCTGCAGAGATCCGACAGCAGCCAGCCAATGCT |
| | | TCTGAGAGTCGTGGGCTCCCAGACCAGCGATTCTATGGGCGAAGAGGACCTGCTCAGCCCTCCTCA |
| | | GGATACAAGCACCGGCCTGGAAGAAGTGATGGAACAACTGAACAACAGCTTCCCCAGCAGCAGAG |
| | | GCAGAAACACCCCTGGCAAGCCCATGCGCGAGGACACCATGTGATAACTCGAGAATCAACCTCTG |
| | | GATTACAAAATTTGTGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCTCCTCCTTGTATAAAT |
| | | CCTGGTTGCTGTCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCTTTCCGGGACTTT |
| | | CGCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGG |
| 37. | CMV | CGTTACATAACTTACGGTAAATGGCCCGCCTGGCTGACCGCCCAACGACCCCCGCCCATTGACGTC |
| | enhancer- | AATAATGACGTATGTTCCCATAGTAACGCCAATAGGGACTTTCCATTGACGTCAATGGGTGGAGTATT |
| | CBA | TACGGTAAACTGCCCACTTGGCAGTACATCAAGTGTATCATATGCCAAGTACGCCCCCTATTGACGT |
| | Promoter- | CAATGACGGTAAATGGCCCGCCTGGCATTATGCCCAGTACATGACCTTATGGGACTTTCCTACTTGG |
| | AAT Exon- | CAGTACATCTACGTATTAGTCATCGCTATTACCATGGTCGAGGTGAGCCCCACGTTCTGCTTCACTCT |
| | hBGI-hBG | CCCCATCTCCCCCCCCTCCCCACCCCCAATTTTGTATTTATTTATTTTTTAATTATTTTGTGCAGCGATG |
| | Exon | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATCGTAAGTACTAGCAGCTACAATCCAGCTACCATTCTGCTTTTATTTTATGGTTGGGATAAG |
| | | GCTGGATTATTCTGAGTCCAAGCTAGGCCCTTTTGCTAATCATGTTCATACCTCTTATCTTCCTCCCAC |
| | | AGCTCCTGGGCAACGTGCTGGTCTGTGTGCTGGCCCATCACTTTGGCAAAGAATTGCGATCGCCAC |
| | | C |
| 38. | CMV | CGTTACATAACTTACGGTAAATGGCCCGCCTGGCTGACCGCCCAACGACCCCCGCCCATTGACGTC |
| | enhancer- | AATAATGACGTATGTTCCCATAGTAACGCCAATAGGGACTTTCCATTGACGTCAATGGGTGGAGTATT |
| | CBA | TACGGTAAACTGCCCACTTGGCAGTACATCAAGTGTATCATATGCCAAGTACGCCCCCTATTGACGT |
| | Promoter- | CAATGACGGTAAATGGCCCGCCTGGCATTATGCCCAGTACATGACCTTATGGGACTTTCCTACTTGG |
| | AAT Exon | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCTCTGGATCCACT |
| | | GCTTAAATACGGACGAGGACAGGGCCCTGTCTCCTCAGCTTCAGGCACCACCACTGACCTGGGAC |
| | | AGTGAATC |
| 39. | CBA | TGGTCGAGGTGAGCCCCACGTTCTGCTTCACTCTCCCCATCTCCCCCCCCTCCCCACCCCCAATTTT |
| | Promoter- | GTATTTATTTATTTTTTAATTATTTTGTGCAGCGATGGGGGCGGGGGGGGGGGGGGCGCGCGCCAGG |
| | AAT Exon | CGGGGCGGGGCGGGGCGAGGGGGGGGCGGGGCGAGGCGGAGAGGTGCGGCGGCAGCCAATCA |
| | | GAGCGGCGCGCTCCGAAAGTTTCCTTTTATGGCGAGGCGGCGGCGGCGGCGGCCCTATAAAAAGC |
| | | GAAGCGCGCGGCGGGCGGGAGTCTCTGGATCCACTGCTTAAATACGGACGAGGACAGGGCCCTGT |
| | | CTCCTCAGCTTCAGGCACCACCACTGACCTGGGACAGTGAATC |
| 40. | CBA | TGGTCGAGGTGAGCCCCACGTTCTGCTTCACTCTCCCCATCTCCCCCCCCTCCCCACCCCCAATTTT |
| | Promoter- | GTATTTATTTATTTTTTAATTATTTTGTGCAGCGATGGGGGCGGGGGGGGGGGGGGCGCGCGCCAGG |
| | AAT Exon- | CGGGGCGGGGCGGGGCGAGGGGCGGGGCGGGGCGAGGCGGAGAGGTGCGGCGGCAGCCAATCA |
| | hBGI-hBG | GAGCGGCGCGCTCCGAAAGTTTCCTTTTATGGCGAGGCGGCGGCGGCGGCGGCCCTATAAAAAGC |
| | Exon | GAAGCGCGCGGCGGGCGGGAGTCTCTGGATCCACTGCTTAAATACGGACGAGGACAGGGCCCTGT |
| | | CTCCTCAGCTTCAGGCACCACCACTGACCTGGGACAGTGAATCGTAAGTACTAGCAGCTACAATCC |
| | | AGCTACCATTCTGCTTTTATTTTATGGTTGGGATAAGGCTGGATTATTCTGAGTCCAAGCTAGGCCCT |
| | | TTTGCTAATCATGTTCATACCTCTTATCTTCCTCCCACAGCTCCTGGGCAACGTGCTGGTCTGTGTGC |
| | | TGGCCCATCACTTTGGCAAAGAATTGCGATCGCCACC |
| 41. | CAG | CGTTACATAACTTACGGTAAATGGCCCGCCTGGCTGACCGCCCAACGACCCCCGCCCATTGACGTC |
| | | AATAATGACGTATGTTCCCATAGTAACGCCAATAGGGACTTTCCATTGACGTCAATGGGTGGAGTATT |
| | | TACGGTAAACTGCCCACTTGGCAGTACATCAAGTGTATCATATGCCAAGTACGCCCCCTATTGACGT |
| | | CAATGACGGTAAATGGCCCGCCTGGCATTATGCCCAGTACATGACCTTATGGGACTTTCCTACTTGG |
| | | CAGTACATCTACGTATTAGTCATCGCTATTACCATGGTCGAGGTGAGCCCCACGTTCTGCTTCACTCT |
| | | CCCCATCTCCCCCCCCTCCCCACCCCCAATTTTGTATTTATTTATTTTTTAATTATTTTGTGCAGCGATG |
| | | GGGGCGGGGGGGGGGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTTTCCTTTTATGGCGA |
| | | GGCGGCGGCGGCGGCGGCCCTATAAAAAGCGAAGCGCGCGGCGGGCGGGAGTCGCTGCGCGCTG |
| | | CCTTCGCCCCGTGCCCCGCTCCGCCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAATTAGCGCTTGGTTTAATG |
| | | ACGGCTTGTTTCTTTTCTGTGGCTGCGTGAAAGCCTTGAGGGGCTCCGGGAGGGCCCTTTGTGCGG |
| | | GGGGAGCGGCTCGGGGGGTGCGTGCGTGTGTGTGTGCGTGGGGAGCGCCGCGTGCGGCTCCGCGC |
| | | TGCCCGGCGGCTGTGAGCGCTGCGGGCGCGGCGCGGGGCTTTGTGCGCTCCGCAGTGTGCGCGAG |
| | | GGGAGCGCGGCCGGGGGCGGTGCCCCGCGGTGCGGGGGGGGCTGCGAGGGGAACAAAGGCTGCG |
| | | TGCGGGGTGTGTGCGTGGGGGGGTGAGCAGGGGGTGTGGGCGCGTCGGTCGGGCTGCAACCCCCC |
| | | CTGCACCCCCCTCCCCGAGTTGCTGAGCACGGCCCGGCTTCGGGTGCGGGGCTCCGTACGGGGCGT |
| | | GGCGCGGGGCTCGCCGTGCCGGGCGGGGGGTGGCGGCAGGTGGGGGTGCCGGGCGGGGCGGGGC |
| | | CGCCTCGGGCCGGGGAGGGCTCGGGGGAGGGGCGCGGCGGCCCCCGGAGCGCCGGCGGCTGTCG |
| | | AGGCGCGGCGAGCCGCAGCCATTGCCTTTTATGGTAATCGTGCGAGAGGGCGCAGGGACTTCCTTT |
| | | GTCCCAAATCTGTGCGGAGCCGAAATCTGGGAGGCGCCGCCGCACCCCCTCTAGCGGGCGCGGGG |
| | | CGAAGCGGTGCGGCGCCGGCAGGAAGGAAATGGGCGGGGAGGGCCTTCGTGCGTCGCCGCGCCG |
| | | CCGTCCCCTTCTCCCTCTCCAGCCTCGGGGCTGTCCGCGGGGGGACGGCTGCCTTCGGGGGGGACG |
| | | GGGCAGGGCGGGGTTCGGCTTCTGGCGTGTGACCGGCGGCTCTAGAGCCTCTGCTAACCATGTTCA |
| | | TGCCTTCTTCTTTTTCCTACAGCTCCTGGGCAACGTGCTGGTTATTGTGCTGTCTCATCATTTTGGCA |
| | | AAGAACGC |
| 42. | NP-CAG- | GTACAGTAGAATTGGTAAAGAGAGTTGTGTAAAATATTGAGTTCGCACATCTTGTTGTCTGATTATTG |
| | EGFP-P2A- | ATTTTTGGCGAAACCATTTGATCATATGACAAGATGTGTATCTACCTTAACTTAATGATTTTGATAAAA |
| | Fluc- | ATCATTAGGTACCTCGCGAATGCATCTAGATCCGACGTTACATAACTTACGGTAAATGGCCCGCCTGG |
| | WPRE3- | CTGACCGCCCAACGACCCCCGCCCATTGACGTCAATAATGACGTATGTTCCCATAGTAACGCCAATA |
| | DTS | GGGACTTTCCATTGACGTCAATGGGTGGAGTATTTACGGTAAACTGCCCACTTGGCAGTACATCAAG |
| | | TGTATCATATGCCAAGTACGCCCCCTATTGACGTCAATGACGGTAAATGGCCCGCCTGGCATTATGCC |
| | | CAGTACATGACCTTATGGGACTTTCCTACTTGGCAGTACATCTACGTATTAGTCATCGCTATTACCATG |
| | | GTCGAGGTGAGCCCCACGTTCTGCTTCACTCTCCCCATCTCCCCCCCCTCCCCACCCCCAATTTTGT |
| | | ATTTATTTATTTTTTAATTATTTTGTGCAGCGATGGGGGCGGGGGGGGGGGGGGCGCGCGCCAGGCG |
| | | GGGCGGGGCGGGGCGAGGGGCGGGGCGGGGCGAGGCGGAGAGGTGCGGCGGCAGCCAATCAGA |
| | | GCGGCGCGCTCCGAAAGTTTCCTTTTATGGCGAGGCGGCGGCGGCGGCGGCCCTATAAAAAGCGA |
| | | AGCGCGCGGCGGGCGGGAGTCGCTGCGCGCTGCCTTCGCCCCGTGCCCCGCTCCGCCGCCGCCTC |
| | | GCGCCGCCCGCCCCGGCTCTGACTGACCGCGTTACTCCCACAGGTGAGCGGGCGGGACGGCCCTT |
| | | CTCCTCCGGGCTGTAATTAGCGCTTGGTTTAATGACGGCTTGTTTCTTTTCTGTGGCTGCGTGAAAG |
| | | CCTTGAGGGGCTCCGGGAGGGCCCTTTGTGCGGGGGGAGCGGCTCGGGGGGTGCGTGCGTGTGTG |
| | | TGTGCGTGGGGAGCGCCGCGTGCGGCTCCGCGCTGCCCGGCGGCTGTGAGCGCTGCGGGCGCGGC |
| | | GCGGGGCTTTGTGCGCTCCGCAGTGTGCGCGAGGGGAGCGCGGCCGGGGGCGGTGCCCCGCGGTG |
| | | CGGGGGGGGCTGCGAGGGGAACAAAGGCTGCGTGCGGGGTGTGTGCGTGGGGGGGTGAGCAGGG |
| | | GGTGTGGGCGCGTCGGTCGGGCTGCAACCCCCCCTGCACCCCCCTCCCCGAGTTGCTGAGCACGG |
| | | CCCGGCTTCGGGTGCGGGGCTCCGTACGGGGCGTGGCGCGGGGCTCGCCGTGCCGGGCGGGGGGT |
| | | GGCGGCAGGTGGGGGTGCCGGGCGGGGCGGGGCCGCCTCGGGCCGGGGAGGGCTCGGGGGAGG |
| | | GGCGCGGCGGCCCCCGGAGCGCCGGCGGCTGTCGAGGCGCGGCGAGCCGCAGCCATTGCCTTTTA |
| | | TGGTAATCGTGCGAGAGGGCGCAGGGACTTCCTTTGTCCCAAATCTGTGCGGAGCCGAAATCTGGG |
| | | AGGCGCCGCCGCACCCCCTCTAGCGGGCGCGGGGCGAAGCGGTGCGGCGCCGGCAGGAAGGAAA |
| | | TGGGCGGGGAGGGCCTTCGTGCGTCGCCGCGCCGCCGTCCCCTTCTCCCTCTCCAGCCTCGGGGCT |
| | | GTCCGCGGGGGGACGGCTGCCTTCGGGGGGGACGGGGCAGGGCGGGGTTCGGCTTCTGGCGTGTG |
| | | ACCGGCGGCTCTAGAGCCTCTGCTAACCATGTTCATGCCTTCTTCTTTTTCCTACAGCTCCTGGGCA |
| | | ACGTGCTGGTTATTGTGCTGTCTCATCATTTTGGCAAAGAACGCGCGATCGCCACCATGGTGAGCAA |
| | | GGGCGAGGAGCTGTTCACCGGGGTGGTGCCCATCCTGGTCGAGCTGGACGGCGACGTAAACGGCC |
| | | ACAAGTTCAGCGTGTCCGGCGAGGGCGAGGGCGATGCCACCTACGGCAAGCTGACCCTGAAGTTC |
| | | ATCTGCACCACCGGCAAGCTGCCCGTGCCCTGGCCCACCCTCGTGACCACCCTGACCTACGGCGTG |
| | | CAGTGCTTCAGCCGCTACCCCGACCACATGAAGCAGCACGACTTCTTCAAGTCCGCCATGCCCGAA |
| | | GGCTACGTCCAGGAGCGCACCATCTTCTTCAAGGACGACGGCAACTACAAGACCCGCGCCGAGGT |
| | | GAAGTTCGAGGGCGACACCCTGGTGAACCGCATCGAGCTGAAGGGCATCGACTTCAAGGAGGACG |
| | | GCAACATCCTGGGGCACAAGCTGGAGTACAACTACAACAGCCACAACGTCTATATCATGGCCGACA |
| | | AGCAGAAGAACGGCATCAAGGTGAACTTCAAGATCCGCCACAACATCGAGGACGGCAGCGTGCAG |
| | | CTCGCCGACCACTACCAGCAGAACACCCCCATCGGCGACGGCCCCGTGCTGCTGCCCGACAACCA |
| | | CTACCTGAGCACCCAGTCCGCCCTGAGCAAAGACCCCAACGAGAAGCGCGATCACATGGTCCTGC |
| | | TGGAGTTCGTGACCGCCGCCGGGATCACTCTCGGCATGGACGAGCTGTACAAGAATCGCGCTACTA |
| | | ACTTCAGCCTGCTGAAGCAGGCTGGAGACGTGGAGGAGAACCCTGGACCTATGGAAGACGCCAAA |
| | | AACATAAAGAAAGGCCCGGCGCCATTCTATCCGCTGGAAGATGGAACCGCTGGAGAGCAACTGCAT |
| | | AAGGCTATGAAGAGATACGCCCTGGTTCCTGGAACAATTGCTTTTACAGATGCACATATCGAGGTGG |
| | | ACATCACTTACGCTGAGTACTTCGAAATGTCCGTTCGGTTGGCAGAAGCTATGAAACGATATGGGCT |
| | | GAATACAAATCACAGAATCGTCGTATGCAGTGAAAACTCTCTTCAATTCTTTATGCCGGTGTTGGGC |
| | | GCGTTATTTATCGGAGTTGCAGTTGCGCCCGCGAACGACATTTATAATGAACGTGAATTGCTCAACA |
| | | GTATGGGCATTTCGCAGCCTACCGTGGTGTTCGTTTCCAAAAAGGGGTTGCAAAAAATTTTGAACG |
| | | TGCAAAAAAAGCTCCCAATCATCCAAAAAATTATTATCATGGATTCTAAAACGGATTACCAGGGATT |
| | | TCAGTCGATGTACACGTTCGTCACATCTCATCTACCTCCCGGTTTTAATGAATACGATTTTGTGCCAG |
| | | AGTCCTTCGATAGGGACAAGACAATTGCACTGATCATGAACTCCTCTGGATCTACTGGTCTGCCTAA |
| | | AGGTGTCGCTCTGCCTCATAGAACTGCCTGCGTGAGATTCTCGCATGCCAGAGATCCTATTTTTGGC |
| | | AATCAAATCATTCCGGATACTGCGATTTTAAGTGTTGTTCCATTCCATCACGGTTTTGGAATGTTTAC |
| | | TACACTCGGATATTTGATATGTGGATTTCGAGTCGTCTTAATGTATAGATTTGAAGAAGAGCTGTTTC |
| | | TGAGGAGCCTTCAGGATTACAAGATTCAAAGTGCGCTGCTGGTGCCAACCCTATTCTCCTTCTTCGC |
| | | CAAAAGCACTCTGATTGACAAATACGATTTATCTAATTTACACGAAATTGCTTCTGGTGGCGCTCCC |
| | | CTCTCTAAGGAAGTCGGGGAAGCGGTTGCCAAGAGGTTCCATCTGCCAGGTATCAGGCAAGGATAT |
| | | GGGCTCACTGAGACTACATCAGCTATTCTGATTACACCCGAGGGGGATGATAAACCGGGCGCGGTC |
| | | GGTAAAGTTGTTCCATTTTTTGAAGCGAAGGTTGTGGATCTGGATACCGGGAAAACGCTGGGCGTT |
| | | AATCAAAGAGGCGAACTGTGTGTGAGAGGTCCTATGATTATGTCCGGTTATGTAAACAATCCGGAA |
| | | GCGACCAACGCCTTGATTGACAAGGATGGATGGCTACATTCTGGAGACATAGCTTACTGGGACGAA |
| | | GACGAACACTTCTTCATCGTTGACCGCCTGAAGTCTCTGATTAAGTACAAAGGCTATCAGGTGGCTC |
| | | CCGCTGAATTGGAATCCATCTTGCTCCAACACCCCAACATCTTCGACGCAGGTGTCGCAGGTCTTCC |
| | | CGACGATGACGCCGGTGAACTTCCCGCCGCCGTTGTTGTTTTGGAGCACGGAAAGACGATGACGG |
| | | AAAAAGAGATCGTGGATTACGTCGCCAGTCAAGTAACAACCGCGAAAAAGTTGCGCGGAGGAGTT |
| | | GTGTTTGTGGACGAAGTACCGAAAGGTCTTACCGGAAAACTCGACGCAAGAAAAATCAGAGAGAT |
| | | CCTCATAAAGGCCAAGAAGGGCGGAAAGATCGCCGTGTAACTCGAGAATCAACCTCTGGATTACAA |
| | | AATTTGTGAAAGATTGACTGGTATTCTTAACTATGTTGCTCCTTTTACGCTATGTGGATACGCTGCTTT |
| | | AATGCCTTTGTATCATGCTATTGCTTCCCGTATGGCTTTCATTTTCTCCTCCTTGTATAAATCCTGGTTG |
| | | CTGTCTCTTTATGAGGAGTTGTGGCCCGTTGTCAGGCAACGTGGCGTGGTGTGCACTGTGTTTGCTG |
| | | ACGCAACCCCCACTGGTTGGGGCATTGCCACCACCTGTCAGCTCCTTTCCGGGACTTTCGCTTTCCC |
| | | CCTCCCTATTGCCACGGCGGAACTCATCGCCGCCTGCCTTGCCCGCTGCTGGACAGGGGCTCGGCT |
| | | GTTGGGCACTGACAATTCCGTGGGGTGTGCCTTCTAGTTGCCAGCCATCTGTTGTTTGCCCCTCCCC |
| | | CGTGCCTTCCTTGACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGACAGCAAGGGGGAG |
| | | GATTGGGAAGACAATAGCAGGCATGCTGGGGATGCGGTGGGCTCTATGGGAAGATGTCTACTGAGC |
| | | TGTGCGATCCCTGCTGGGGACTTTCCGCTGGGGACTTTCCGCTGGGGACTTTCCGCCTTCAGCTAA |
| | | GGAAGCTACCAATATTTAGAGGTACATTTTGTTCTAGAACAAAATGTACCGGTACATTTTGTTCTGGT |
| | | ACATTTTGTTCTATTAAGGACAACGTAAGTATAGCGCATAGACACGTGGCGCCATCGGATCCCGGGT |
| | | GGCTTGTTGTCCACAACCATTAAACCTTAAAAGCTTTAAAAGCCTTATATATTCTTTTTTTTCTTATAA |
| | | AACTTAAAACCTTAGAGGCTATTTAAGTTGCTGATTTATATTAATTTTATTGTTCAAACATGAGAGCTT |
| | | AGTACGTGAAACATGAGAGCTTAGTACATTAGCCATGAGAGCTTAGTACATTAGCCATGAGGGTTTA |
| | | GTTCATTAAACATGAGAGCTTAGTACATTAAACATGAGAGCTTAGTACATTAAACATGAGAGCTTAG |
| | | TACATACTATCAACAGGTTGAACTGCTGATCT |
| 43. | NP-CBA- | TGCATGCGGCCGCCGTTACATAACTTACGGTAAATGGCCCGCCTGGCTGACCGCCCAACGACCCCC |
| | hBGi- | GCCCATTGACGTCAATAATGACGTATGTTCCCATAGTAACGCCAATAGGGACTTTCCATTGACGTCA |
| | EGFP-P2A- | ATGGGTGGAGTATTTACGGTAAACTGCCCACTTGGCAGTACATCAAGTGTATCATATGCCAAGTACG |
| | Fluc- | CCCCCTATTGACGTCAATGACGGTAAATGGCCCGCCTGGCATTATGCCCAGTACATGACCTTATGGG |
| | WPRE3- | ACTTTCCTACTTGGCAGTACATCTACGTATTAGTCATCGCTATTACCATGGTCGAGGTGAGCCCCACG |
| | DTS | TTCTGCTTCACTCTCCCCATCTCCCCCCCCTCCCCACCCCCAATTTTGTATTTATTTATTTTTTAATTAT |
| | | TTTGTGCAGCGATGGGGGCGGGGGGGGGGGGGGCGCGCGCCAGGCGGGGGGGGCGGGGCGAGG |
| | | GGCGGGGCGGGGCGAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCGCGGCGGGCGGGAG |
| | | TCTCTGGATCCACTGCTTAAATACGGACGAGGACAGGGCCCTGTCTCCTCAGCTTCAGGCACCACC |
| | | ACTGACCTGGGACAGTGAATCGTAAGTACTAGCAGCTACAATCCAGCTACCATTCTGCTTTTATTTTA |
| | | TGGTTGGGATAAGGCTGGATTATTCTGAGTCCAAGCTAGGCCCTTTTGCTAATCATGTTCATACCTCT |
| | | TATCTTCCTCCCACAGCTCCTGGGCAACGTGCTGGTCTGTGTGCTGGCCCATCACTTTGGCAAAGAA |
| | | TTGCGATCGCCACCATGGTGAGCAAGGGCGAGGAGCTGTTCACCGGGGTGGTGCCCATCCTGGTCG |
| | | AGCTGGACGGCGACGTAAACGGCCACAAGTTCAGCGTGTCCGGCGAGGGCGAGGGCGATGCCACC |
| | | TACGGCAAGCTGACCCTGAAGTTCATCTGCACCACCGGCAAGCTGCCCGTGCCCTGGCCCACCCTC |
| | | GTGACCACCCTGACCTACGGCGTGCAGTGCTTCAGCCGCTACCCCGACCACATGAAGCAGCACGA |
| | | CTTCTTCAAGTCCGCCATGCCCGAAGGCTACGTCCAGGAGCGCACCATCTTCTTCAAGGACGACGG |
| | | CAACTACAAGACCCGCGCCGAGGTGAAGTTCGAGGGCGACACCCTGGTGAACCGCATCGAGCTGA |
| | | AGGGCATCGACTTCAAGGAGGACGGCAACATCCTGGGGCACAAGCTGGAGTACAACTACAACAGC |
| | | CACAACGTCTATATCATGGCCGACAAGCAGAAGAACGGCATCAAGGTGAACTTCAAGATCCGCCAC |
| | | AACATCGAGGACGGCAGCGTGCAGCTCGCCGACCACTACCAGCAGAACACCCCCATCGGCGACGG |
| | | CCCCGTGCTGCTGCCCGACAACCACTACCTGAGCACCCAGTCCGCCCTGAGCAAAGACCCCAACG |
| | | AGAAGCGCGATCACATGGTCCTGCTGGAGTTCGTGACCGCCGCCGGGATCACTCTCGGCATGGACG |
| | | AGCTGTACAAGAATCGCGCTACTAACTTCAGCCTGCTGAAGCAGGCTGGAGACGTGGAGGAGAAC |
| | | CCTGGACCTATGGAAGACGCCAAAAACATAAAGAAAGGCCCGGCGCCATTCTATCCGCTGGAAGAT |
| | | GGAACCGCTGGAGAGCAACTGCATAAGGCTATGAAGAGATACGCCCTGGTTCCTGGAACAATTGCT |
| | | TTTACAGATGCACATATCGAGGTGGACATCACTTACGCTGAGTACTTCGAAATGTCCGTTCGGTTGG |
| | | CAGAAGCTATGAAACGATATGGGCTGAATACAAATCACAGAATCGTCGTATGCAGTGAAAACTCTCT |
| | | TCAATTCTTTATGCCGGTGTTGGGCGCGTTATTTATCGGAGTTGCAGTTGCGCCCGCGAACGACATTT |
| | | ATAATGAACGTGAATTGCTCAACAGTATGGGCATTTCGCAGCCTACCGTGGTGTTCGTTTCCAAAAA |
| | | GGGGTTGCAAAAAATTTTGAACGTGCAAAAAAAGCTCCCAATCATCCAAAAAATTATTATCATGGAT |
| | | TCTAAAACGGATTACCAGGGATTTCAGTCGATGTACACGTTCGTCACATCTCATCTACCTCCCGGTTT |
| | | TAATGAATACGATTTTGTGCCAGAGTCCTTCGATAGGGACAAGACAATTGCACTGATCATGAACTCC |
| | | TCTGGATCTACTGGTCTGCCTAAAGGTGTCGCTCTGCCTCATAGAACTGCCTGCGTGAGATTCTCGC |
| | | ATGCCAGAGATCCTATTTTTGGCAATCAAATCATTCCGGATACTGCGATTTTAAGTGTTGTTCCATTC |
| | | CATCACGGTTTTGGAATGTTTACTACACTCGGATATTTGATATGTGGATTTCGAGTCGTCTTAATGTAT |
| | | AGATTTGAAGAAGAGCTGTTTCTGAGGAGCCTTCAGGATTACAAGATTCAAAGTGCGCTGCTGGTG |
| | | CCAACCCTATTCTCCTTCTTCGCCAAAAGCACTCTGATTGACAAATACGATTTATCTAATTTACACGA |
| | | AATTGCTTCTGGTGGCGCTCCCCTCTCTAAGGAAGTCGGGGAAGCGGTTGCCAAGAGGTTCCATCT |
| | | GCCAGGTATCAGGCAAGGATATGGGCTCACTGAGACTACATCAGCTATTCTGATTACACCCGAGGGG |
| | | GATGATAAACCGGGCGCGGTCGGTAAAGTTGTTCCATTTTTTGAAGCGAAGGTTGTGGATCTGGATA |
| | | CCGGGAAAACGCTGGGCGTTAATCAAAGAGGCGAACTGTGTGTGAGAGGTCCTATGATTATGTCCG |
| | | GTTATGTAAACAATCCGGAAGCGACCAACGCCTTGATTGACAAGGATGGATGGCTACATTCTGGAG |
| | | ACATAGCTTACTGGGACGAAGACGAACACTTCTTCATCGTTGACCGCCTGAAGTCTCTGATTAAGTA |
| | | CAAAGGCTATCAGGTGGCTCCCGCTGAATTGGAATCCATCTTGCTCCAACACCCCAACATCTTCGAC |
| | | GCAGGTGTCGCAGGTCTTCCCGACGATGACGCCGGTGAACTTCCCGCCGCCGTTGTTGTTTTGGAG |
| | | CACGGAAAGACGATGACGGAAAAAGAGATCGTGGATTACGTCGCCAGTCAAGTAACAACCGCGAA |
| | | AAAGTTGCGCGGAGGAGTTGTGTTTGTGGACGAAGTACCGAAAGGTCTTACCGGAAAACTCGACG |
| | | CAAGAAAAATCAGAGAGATCCTCATAAAGGCCAAGAAGGGCGGAAAGATCGCCGTGTAACTCGAG |
| | | AATCAACCTCTGGATTACAAAATTTGTGAAAGATTGACTGGTATTCTTAACTATGTTGCTCCTTTTAC |
| | | GCTATGTGGATACGCTGCTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGGGGACTTTCCGCTG |
| | | GGGACTTTCCGCCTTCAGCTAAGGAAGCTACCAATATTTAGAGGTACATTTTGTTCTAGAACAAAAT |
| | | GTACCGGTACATTTTGTTCTGGTACATTTTGTTCTATTAAGGACAACGTAAGTATAGCGCATAGACAC |
| | | GTGGCGCCATCGGATCCCGGGTGGCTTGTTGTCCACAACCATTAAACCTTAAAAGCTTTAAAAGCCT |
| | | TATATATTCTTTTTTTTCTTATAAAACTTAAAACCTTAGAGGCTATTTAAGTTGCTGATTTATATTAATT |
| | | TTATTGTTCAAACATGAGAGCTTAGTACGTGAAACATGAGAGCTTAGTACATTAGCCATGAGAGCTTA |
| | | GTACATTAGCCATGAGGGTTTAGTTCATTAAACATGAGAGCTTAGTACATTAAACATGAGAGCTTAG |
| | | TACATTAAACATGAGAGCTTAGTACATACTATCAACAGGTTGAACTGCTGATCTGTACAGTAGAATT |
| | | GGTAAAGAGAGTTGTGTAAAATATTGAGTTCGCACATCTTGTTGTCTGATTATTGATTTTTGGCGAAA |
| | | CCATTTGATCATATGACAAGATGTGTATCTACCTTAACTTAATGATTTTGATAAAAATCATTA |
| 44. | hNPHS1- | CACCTGAGGTCAGGAGTTCGAGACCAGCGTGGCCAACATGATGAAACCCCGTCTCTAGTAAAAATA |
| | hBGi | CAAAAATTAGCCAGGCATGGTGCTATATACCTGTAGCACCAGCTACTTGGGAGACAGAGGTGGGAG |
| | | AATTACTTGAACCTGGGAGGTTCAAGCCATGGGAGGTGGAAGTTGCAGTGAGCCGAGATGCCACT |
| | | GCACTCCAGCCTGAGCAACAGAGCAAGACTATCTCAAGAAAAGAAAGAAAGAAAGAAAGAGACT |
| | | TGCCAAGGTCATGTATCAGGGCAAGGAAGAGCTGGGGGCCCAGCTGGCTGCTCCCCTGCTGAGCT |
| | | GGGAGACCACCTTGATCTGACTTCTCCCATCTTCCCAGCCTAAGCCAGGCCCTGGGGTCACGGAGG |
| | | CTGGGGAGGCACCGAGGAACGCGCCTGGCATGTGCTGACAGGGGATTTTATGCTCCAGCTGGGCC |
| | | AGCTGGGAGGAGCCTGCTGGGCAGAGGCCAGAGCTGGGGGCTCTGGAAGGTACCTGGGGGAGGT |
| | | TGCACTGTGAGAATGAGCTCAAGCTGGGTCAGAGAGCAGGGCTGACTCTGCCAGTGCCTGCATCA |
| | | GCCTCATCGCTCTCCTAGGCTCCTGGCCTGCTGGACTCTGGGCTGCAGGTCCTTCTTGAAAGGCTGT |
| | | GAGTAGTGAGACAAGGAGCAGGAGTGAGGGGTGGCAGGAGAGAAGATAGAGATTGAGAGAGAGA |
| | | GAGAGAGAGAGACAGAGAGAGAGGAAGAGACAGAGACAAAAGGAGAGAGAACGGCTTAGACAA |
| | | GGAGAGAAAGATGGAAAGATAAAGAGACTGGGCGCAGTGGCTCACGCCTGTAATCCCAACACTTG |
| | | GGGAGGCCAAGGTGGGAGGATGGCTTGAAGGAAAGAGTCTGAGATCAACCTGGCCAACATAGTGA |
| | | GACCCTGTCTCTAAAAAAAAAAAAAAGAAAAAAAAAAGAAAAAAGAAAAAAAAGTTTTTTTAAA |
| | | GAGACAGAGAAAGAGACTCAGAGATTGAGACTGAGAGCAAGACAGAGAGAGATACTCACAGGGA |
| | | AGAGGGGAAGAGGAAAACGAGAAAGGGAGGAGAGTAACGGAAAGAGATAAAAAAGAAAAGCAG |
| | | GTGGCAGAGACACACAGAGAGGGACCCAGAGAAAGCCAGACAGACGCAGGTGGCTGGCAGCGG |
| | | GCGCTGTGGGGGTCACAGTAGGGGGACCTGTGTCTGGATCCACTGCTTAAATACGGACGAGGACA |
| | | GGGCCCTGTCTCCTCAGCTTCAGGCACCACCACTGACCTGGGACAGTGAATCGTAAGTACTAGCAG |
| | | CTACAATCCAGCTACCATTCTGCTTTTATTTTATGGTTGGGATAAGGCTGGATTATTCTGAGTCCAAG |
| | | CTAGGCCCTTTTGCTAATCATGTTCATACCTCTTATCTTCCTCCCACAGCTCCTGGGCAACGTGCTGG |
| | | TCTGTGTGCTGGCCCATCACTTTGGCAAAGAATTGCGATCGCCACC |
| 45. | NP- | GCCACCTGAGGTCAGGAGTTCGAGACCAGCGTGGCCAACATGATGAAACCCCGTCTCTAGTAAAA |
| | hNPHS1- | ATACAAAAATTAGCCAGGCATGGTGCTATATACCTGTAGCACCAGCTACTTGGGAGACAGAGGTGG |
| | hBGi- | GAGAATTACTTGAACCTGGGAGGTTCAAGCCATGGGAGGTGGAAGTTGCAGTGAGCCGAGATGCC |
| | EGFP-P2A- | ACTGCACTCCAGCCTGAGCAACAGAGCAAGACTATCTCAAGAAAAGAAAGAAAGAAAGAAAGAG |
| | Fluc- | ACTTGCCAAGGTCATGTATCAGGGCAAGGAAGAGCTGGGGGCCCAGCTGGCTGCTCCCCTGCTGA |
| | WPRE3- | GCTGGGAGACCACCTTGATCTGACTTCTCCCATCTTCCCAGCCTAAGCCAGGCCCTGGGGTCACGG |
| | DTS | AGGCTGGGGAGGCACCGAGGAACGCGCCTGGCATGTGCTGACAGGGGATTTTATGCTCCAGCTGG |
| | | GCCAGCTGGGAGGAGCCTGCTGGGCAGAGGCCAGAGCTGGGGGCTCTGGAAGGTACCTGGGGGA |
| | | GGTTGCACTGTGAGAATGAGCTCAAGCTGGGTCAGAGAGCAGGGCTGACTCTGCCAGTGCCTGCA |
| | | TCAGCCTCATCGCTCTCCTAGGCTCCTGGCCTGCTGGACTCTGGGCTGCAGGTCCTTCTTGAAAGGC |
| | | TGTGAGTAGTGAGACAAGGAGCAGGAGTGAGGGGTGGCAGGAGAGAAGATAGAGATTGAGAGAG |
| | | AGAGAGAGAGAGAGACAGAGAGAGAGGAAGAGACAGAGACAAAAGGAGAGAGAACGGCTTAGA |
| | | CAAGGAGAGAAAGATGGAAAGATAAAGAGACTGGGCGCAGTGGCTCACGCCTGTAATCCCAACAC |
| | | TTGGGGAGGCCAAGGTGGGAGGATGGCTTGAAGGAAAGAGTCTGAGATCAACCTGGCCAACATAG |
| | | TGAGACCCTGTCTCTAAAAAAAAAAAAAAGAAAAAAAAAAGAAAAAAGAAAAAAAAGTTTTTTT |
| | | AAAGAGACAGAGAAAGAGACTCAGAGATTGAGACTGAGAGCAAGACAGAGAGAGATACTCACAG |
| | | GGAAGAGGGGAAGAGGAAAACGAGAAAGGGAGGAGAGTAACGGAAAGAGATAAAAAAGAAAAG |
| | | CAGGTGGCAGAGACACACAGAGAGGGACCCAGAGAAAGCCAGACAGACGCAGGTGGCTGGCAG |
| | | CGGGCGCTGTGGGGGTCACAGTAGGGGGACCTGTGTCTGGATCCACTGCTTAAATACGGACGAGG |
| | | ACAGGGCCCTGTCTCCTCAGCTTCAGGCACCACCACTGACCTGGGACAGTGAATCGTAAGTACTAG |
| | | CAGCTACAATCCAGCTACCATTCTGCTTTTATTTTATGGTTGGGATAAGGCTGGATTATTCTGAGTCC |
| | | AAGCTAGGCCCTTTTGCTAATCATGTTCATACCTCTTATCTTCCTCCCACAGCTCCTGGGCAACGTGC |
| | | TGGTCTGTGTGCTGGCCCATCACTTTGGCAAAGAATTGCGATCGCCACCATGGTGAGCAAGGGCGA |
| | | GGAGCTGTTCACCGGGGTGGTGCCCATCCTGGTCGAGCTGGACGGCGACGTAAACGGCCACAAGT |
| | | TCAGCGTGTCCGGCGAGGGCGAGGGCGATGCCACCTACGGCAAGCTGACCCTGAAGTTCATCTGC |
| | | ACCACCGGCAAGCTGCCCGTGCCCTGGCCCACCCTCGTGACCACCCTGACCTACGGCGTGCAGTGC |
| | | TTCAGCCGCTACCCCGACCACATGAAGCAGCACGACTTCTTCAAGTCCGCCATGCCCGAAGGCTAC |
| | | GTCCAGGAGCGCACCATCTTCTTCAAGGACGACGGCAACTACAAGACCCGCGCCGAGGTGAAGTT |
| | | CGAGGGCGACACCCTGGTGAACCGCATCGAGCTGAAGGGCATCGACTTCAAGGAGGACGGCAAC |
| | | ATCCTGGGGCACAAGCTGGAGTACAACTACAACAGCCACAACGTCTATATCATGGCCGACAAGCAG |
| | | AAGAACGGCATCAAGGTGAACTTCAAGATCCGCCACAACATCGAGGACGGCAGCGTGCAGCTCGC |
| | | CGACCACTACCAGCAGAACACCCCCATCGGCGACGGCCCCGTGCTGCTGCCCGACAACCACTACCT |
| | | GAGCACCCAGTCCGCCCTGAGCAAAGACCCCAACGAGAAGCGCGATCACATGGTCCTGCTGGAGT |
| | | TCGTGACCGCCGCCGGGATCACTCTCGGCATGGACGAGCTGTACAAGAATCGCGCTACTAACTTCA |
| | | GCCTGCTGAAGCAGGCTGGAGACGTGGAGGAGAACCCTGGACCTATGGAAGACGCCAAAAACATA |
| | | AAGAAAGGCCCGGCGCCATTCTATCCGCTGGAAGATGGAACCGCTGGAGAGCAACTGCATAAGGC |
| | | TATGAAGAGATACGCCCTGGTTCCTGGAACAATTGCTTTTACAGATGCACATATCGAGGTGGACATC |
| | | ACTTACGCTGAGTACTTCGAAATGTCCGTTCGGTTGGCAGAAGCTATGAAACGATATGGGCTGAATA |
| | | CAAATCACAGAATCGTCGTATGCAGTGAAAACTCTCTTCAATTCTTTATGCCGGTGTTGGGCGCGTT |
| | | ATTTATCGGAGTTGCAGTTGCGCCCGCGAACGACATTTATAATGAACGTGAATTGCTCAACAGTATG |
| | | GGCATTTCGCAGCCTACCGTGGTGTTCGTTTCCAAAAAGGGGTTGCAAAAAATTTTGAACGTGCAA |
| | | AAAAAGCTCCCAATCATCCAAAAAATTATTATCATGGATTCTAAAACGGATTACCAGGGATTTCAGT |
| | | CGATGTACACGTTCGTCACATCTCATCTACCTCCCGGTTTTAATGAATACGATTTTGTGCCAGAGTCC |
| | | TTCGATAGGGACAAGACAATTGCACTGATCATGAACTCCTCTGGATCTACTGGTCTGCCTAAAGGTG |
| | | TCGCTCTGCCTCATAGAACTGCCTGCGTGAGATTCTCGCATGCCAGAGATCCTATTTTTGGCAATCA |
| | | AATCATTCCGGATACTGCGATTTTAAGTGTTGTTCCATTCCATCACGGTTTTGGAATGTTTACTACAC |
| | | TCGGATATTTGATATGTGGATTTCGAGTCGTCTTAATGTATAGATTTGAAGAAGAGCTGTTTCTGAGG |
| | | AGCCTTCAGGATTACAAGATTCAAAGTGCGCTGCTGGTGCCAACCCTATTCTCCTTCTTCGCCAAAA |
| | | GCACTCTGATTGACAAATACGATTTATCTAATTTACACGAAATTGCTTCTGGTGGCGCTCCCCTCTCT |
| | | AAGGAAGTCGGGGAAGCGGTTGCCAAGAGGTTCCATCTGCCAGGTATCAGGCAAGGATATGGGCT |
| | | CACTGAGACTACATCAGCTATTCTGATTACACCCGAGGGGGATGATAAACCGGGCGCGGTCGGTAA |
| | | AGTTGTTCCATTTTTTGAAGCGAAGGTTGTGGATCTGGATACCGGGAAAACGCTGGGCGTTAATCA |
| | | AAGAGGCGAACTGTGTGTGAGAGGTCCTATGATTATGTCCGGTTATGTAAACAATCCGGAAGCGAC |
| | | CAACGCCTTGATTGACAAGGATGGATGGCTACATTCTGGAGACATAGCTTACTGGGACGAAGACGA |
| | | ACACTTCTTCATCGTTGACCGCCTGAAGTCTCTGATTAAGTACAAAGGCTATCAGGTGGCTCCCGCT |
| | | GAATTGGAATCCATCTTGCTCCAACACCCCAACATCTTCGACGCAGGTGTCGCAGGTCTTCCCGAC |
| | | GATGACGCCGGTGAACTTCCCGCCGCCGTTGTTGTTTTGGAGCACGGAAAGACGATGACGGAAAA |
| | | AGAGATCGTGGATTACGTCGCCAGTCAAGTAACAACCGCGAAAAAGTTGCGCGGAGGAGTTGTGT |
| | | TTGTGGACGAAGTACCGAAAGGTCTTACCGGAAAACTCGACGCAAGAAAAATCAGAGAGATCCTC |
| | | ATAAAGGCCAAGAAGGGCGGAAAGATCGCCGTGTAACTCGAGAATCAACCTCTGGATTACAAAATT |
| | | TGTGAAAGATTGACTGGTATTCTTAACTATGTTGCTCCTTTTACGCTATGTGGATACGCTGCTTTAATG |
| | | CCTTTGTATCATGCTATTGCTTCCCGTATGGCTTTCATTTTCTCCTCCTTGTATAAATCCTGGTTGCTGT |
| | | CTCTTTATGAGGAGTTGTGGCCCGTTGTCAGGCAACGTGGCGTGGTGTGCACTGTGTTTGCTGACG |
| | | CAACCCCCACTGGTTGGGGCATTGCCACCACCTGTCAGCTCCTTTCCGGGACTTTCGCTTTCCCCCT |
| | | CCCTATTGCCACGGCGGAACTCATCGCCGCCTGCCTTGCCCGCTGCTGGACAGGGGCTCGGCTGTT |
| | | GGGCACTGACAATTCCGTGGGGTGTGCCTTCTAGTTGCCAGCCATCTGTTGTTTGCCCCTCCCCCGT |
| | | GCCTTCCTTGACCCTGGAAGGTGCCACTCCCACTGTCCTTTCCTAATAAAATGAGGAAATTGCATCG |
| | | CATTGTCTGAGTAGGTGTCATTCTATTCTGGGGGGTGGGGTGGGGCAGGACAGCAAGGGGGAGGAT |
| | | TGGGAAGACAATAGCAGGCATGCTGGGGATGCGGTGGGCTCTATGGGAAGATGTCTACTGAGCTGT |
| | | GCGATCCCTGCTGGGGACTTTCCGCTGGGGACTTTCCGCTGGGGACTTTCCGCCTTCAGCTAAGGA |
| | | AGCTACCAATATTTAGAGGTACATTTTGTTCTAGAACAAAATGTACCGGTACATTTTGTTCTGGTACA |
| | | TTTTGTTCTATTAAGGACAACGTAAGTATAGCGCATAGACACTGGCTTGTTGTCCACAACCATTAAA |
| | | CCTTAAAAGCTTTAAAAGCCTTATATATTCTTTTTTTTCTTATAAAACTTAAAACCTTAGAGGCTATTT |
| | | AAGTTGCTGATTTATATTAATTTTATTGTTCAAACATGAGAGCTTAGTACGTGAAACATGAGAGCTTA |
| | | GTACATTAGCCATGAGAGCTTAGTACATTAGCCATGAGGGTTTAGTTCATTAAACATGAGAGCTTAGT |
| | | ACATTAAACATGAGAGCTTAGTACATTAAACATGAGAGCTTAGTACATACTATCAACAGGTTGAACT |
| | | GCTGATCTGTACAGTAGAATTGGTAAAGAGAGTTGTGTAAAATATTGAGTTCGCACATCTTGTTGTC |
| | | TGATTATTGATTTTTGGCGAAACCATTTGATCATATGACAAGATGTGTATCTACCTTAACTTAATGATT |
| | | TTGATAAAAATCATTA |
| 46. | MHCK | CTAGAAGCTGCATGTCTAAGCTAGACCCTTCAGATTAAAAATAACTGAGGTAAGGGCCTGGGTAGG |
| | Promoter | GGAGGTGGTGTGAGACGCTCCTGTCTCTCCTCTATCTGCCCATCGGCCCTTTGGGGAGGAGGAATG |
| | | TGCCCAAGGACTAAAAAAAGGCCATGGAGCCAGAGGGGCGAGGGCAACAGACCTTTCATGGGCA |
| | | AACCTTGGGGCCCTGCTGTCTAGCATGCCCCACTACGGGTCTAGGCTGCCCATGTAAGGAGGCAAG |
| | | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACC |
| 47. | NP-CBA- | TGCATGCGGCCGCCGTTACATAACTTACGGTAAATGGCCCGCCTGGCTGACCGCCCAACGACCCCC |
| | hBGi- | GCCCATTGACGTCAATAATGACGTATGTTCCCATAGTAACGCCAATAGGGACTTTCCATTGACGTCA |
| | EGFP-P2A- | ATGGGTGGAGTATTTACGGTAAACTGCCCACTTGGCAGTACATCAAGTGTATCATATGCCAAGTACG |
| | Fluc- | CCCCCTATTGACGTCAATGACGGTAAATGGCCCGCCTGGCATTATGCCCAGTACATGACCTTATGGG |
| | WPRE3- | ACTTTCCTACTTGGCAGTACATCTACGTATTAGTCATCGCTATTACCATGGTCGAGGTGAGCCCCACG |
| | DTS | TTCTGCTTCACTCTCCCCATCTCCCCCCCCTCCCCACCCCCAATTTTGTATTTATTTATTTTTTAATTAT |
| | | TTTGTGCAGCGATGGGGGCGGGGGGGGGGGGGGCGCGCGCCAGGCGGGGCGGGGCGGGGCGAGG |
| | | GGCGGGGCGGGGCGAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCGCGGCGGGCGGGAG |
| | | TCTCTGGATCCACTGCTTAAATACGGACGAGGACAGGGCCCTGTCTCCTCAGCTTCAGGCACCACC |
| | | ACTGACCTGGGACAGTGAATCGTAAGTACTAGCAGCTACAATCCAGCTACCATTCTGCTTTTATTTTA |
| | | TGGTTGGGATAAGGCTGGATTATTCTGAGTCCAAGCTAGGCCCTTTTGCTAATCATGTTCATACCTCT |
| | | TATCTTCCTCCCACAGCTCCTGGGCAACGTGCTGGTCTGTGTGCTGGCCCATCACTTTGGCAAAGAA |
| | | TTGCGATCGCCACCATGGTGAGCAAGGGCGAGGAGCTGTTCACCGGGGTGGTGCCCATCCTGGTCG |
| | | AGCTGGACGGCGACGTAAACGGCCACAAGTTCAGCGTGTCCGGCGAGGGCGAGGGCGATGCCACC |
| | | TACGGCAAGCTGACCCTGAAGTTCATCTGCACCACCGGCAAGCTGCCCGTGCCCTGGCCCACCCTC |
| | | GTGACCACCCTGACCTACGGCGTGCAGTGCTTCAGCCGCTACCCCGACCACATGAAGCAGCACGA |
| | | CTTCTTCAAGTCCGCCATGCCCGAAGGCTACGTCCAGGAGCGCACCATCTTCTTCAAGGACGACGG |
| | | CAACTACAAGACCCGCGCCGAGGTGAAGTTCGAGGGCGACACCCTGGTGAACCGCATCGAGCTGA |
| | | AGGGCATCGACTTCAAGGAGGACGGCAACATCCTGGGGCACAAGCTGGAGTACAACTACAACAGC |
| | | CACAACGTCTATATCATGGCCGACAAGCAGAAGAACGGCATCAAGGTGAACTTCAAGATCCGCCAC |
| | | AACATCGAGGACGGCAGCGTGCAGCTCGCCGACCACTACCAGCAGAACACCCCCATCGGCGACGG |
| | | CCCCGTGCTGCTGCCCGACAACCACTACCTGAGCACCCAGTCCGCCCTGAGCAAAGACCCCAACG |
| | | AGAAGCGCGATCACATGGTCCTGCTGGAGTTCGTGACCGCCGCCGGGATCACTCTCGGCATGGACG |
| | | AGCTGTACAAGAATCGCGCTACTAACTTCAGCCTGCTGAAGCAGGCTGGAGACGTGGAGGAGAAC |
| | | CCTGGACCTATGGAAGACGCCAAAAACATAAAGAAAGGCCCGGCGCCATTCTATCCGCTGGAAGAT |
| | | GGAACCGCTGGAGAGCAACTGCATAAGGCTATGAAGAGATACGCCCTGGTTCCTGGAACAATTGCT |
| | | TTTACAGATGCACATATCGAGGTGGACATCACTTACGCTGAGTACTTCGAAATGTCCGTTCGGTTGG |
| | | CAGAAGCTATGAAACGATATGGGCTGAATACAAATCACAGAATCGTCGTATGCAGTGAAAACTCTCT |
| | | TCAATTCTTTATGCCGGTGTTGGGCGCGTTATTTATCGGAGTTGCAGTTGCGCCCGCGAACGACATTT |
| | | ATAATGAACGTGAATTGCTCAACAGTATGGGCATTTCGCAGCCTACCGTGGTGTTCGTTTCCAAAAA |
| | | GGGGTTGCAAAAAATTTTGAACGTGCAAAAAAAGCTCCCAATCATCCAAAAAATTATTATCATGGAT |
| | | TCTAAAACGGATTACCAGGGATTTCAGTCGATGTACACGTTCGTCACATCTCATCTACCTCCCGGTTT |
| | | TAATGAATACGATTTTGTGCCAGAGTCCTTCGATAGGGACAAGACAATTGCACTGATCATGAACTCC |
| | | TCTGGATCTACTGGTCTGCCTAAAGGTGTCGCTCTGCCTCATAGAACTGCCTGCGTGAGATTCTCGC |
| | | ATGCCAGAGATCCTATTTTTGGCAATCAAATCATTCCGGATACTGCGATTTTAAGTGTTGTTCCATTC |
| | | CATCACGGTTTTGGAATGTTTACTACACTCGGATATTTGATATGTGGATTTCGAGTCGTCTTAATGTAT |
| | | AGATTTGAAGAAGAGCTGTTTCTGAGGAGCCTTCAGGATTACAAGATTCAAAGTGCGCTGCTGGTG |
| | | CCAACCCTATTCTCCTTCTTCGCCAAAAGCACTCTGATTGACAAATACGATTTATCTAATTTACACGA |
| | | AATTGCTTCTGGTGGCGCTCCCCTCTCTAAGGAAGTCGGGGAAGCGGTTGCCAAGAGGTTCCATCT |
| | | GCCAGGTATCAGGCAAGGATATGGGCTCACTGAGACTACATCAGCTATTCTGATTACACCCGAGGGG |
| | | GATGATAAACCGGGCGCGGTCGGTAAAGTTGTTCCATTTTTTGAAGCGAAGGTTGTGGATCTGGATA |
| | | CCGGGAAAACGCTGGGCGTTAATCAAAGAGGCGAACTGTGTGTGAGAGGTCCTATGATTATGTCCG |
| | | GTTATGTAAACAATCCGGAAGCGACCAACGCCTTGATTGACAAGGATGGATGGCTACATTCTGGAG |
| | | ACATAGCTTACTGGGACGAAGACGAACACTTCTTCATCGTTGACCGCCTGAAGTCTCTGATTAAGTA |
| | | CAAAGGCTATCAGGTGGCTCCCGCTGAATTGGAATCCATCTTGCTCCAACACCCCAACATCTTCGAC |
| | | GCAGGTGTCGCAGGTCTTCCCGACGATGACGCCGGTGAACTTCCCGCCGCCGTTGTTGTTTTGGAG |
| | | CACGGAAAGACGATGACGGAAAAAGAGATCGTGGATTACGTCGCCAGTCAAGTAACAACCGCGAA |
| | | AAAGTTGCGCGGAGGAGTTGTGTTTGTGGACGAAGTACCGAAAGGTCTTACCGGAAAACTCGACG |
| | | CAAGAAAAATCAGAGAGATCCTCATAAAGGCCAAGAAGGGCGGAAAGATCGCCGTGTAACTCGAG |
| | | AATCAACCTCTGGATTACAAAATTTGTGAAAGATTGACTGGTATTCTTAACTATGTTGCTCCTTTTAC |
| | | GCTATGTGGATACGCTGCTTTAATGCCTTTGTATCATGCTATTGCTTCCCGTATGGCTTTCATTTTCTCC |
| | | TCCTTGTATAAATCCTGGTTGCTGTCTCTTTATGAGGAGTTGTGGCCCGTTGTCAGGCAACGTGGCG |
| | | TGGTGTGCACTGTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGGGTGTGCCTTCTAGTTGCCAGC |
| | | CATCTGTTGTTTGCCCCTCCCCCGTGCCTTCCTTGACCCTGGAAGGTGCCACTCCCACTGTCCTTTC |
| | | CTAATAAAATGAGGAAATTGCATCGCATTGTCTGAGTAGGTGTCATTCTATTCTGGGGGGTGGGGTG |
| | | GGGCAGGACAGCAAGGGGGAGGATTGGGAAGACAATAGCAGGCATGCTGGGGATGCGGTGGGCT |
| | | CTATGGGAAGATGTCTACTGAGCTGTGCGATCCCTGCTGGGGACTTTCCGCTGGGGACTTTCCGCTG |
| | | GGGACTTTCCGCCTTCAGCTAAGGAAGCTACCAATATTTAGAGGTACATTTTGTTCTAGAACAAAAT |
| | | GTACCGGTACATTTTGTTCTGGTACATTTTGTTCTATTAAGGACAACGTAAGTATAGCGCATAGACAC |
| | | GTGGCGCCATCGGATCCCGGGTGGCTTGTTGTCCACAACCATTAAACCTTAAAAGCTTTAAAAGCCT |
| | | TATATATTCTTTTTTTTCTTATAAAACTTAAAACCTTAGAGGCTATTTAAGTTGCTGATTTATATTAATT |
| | | TTATTGTTCAAACATGAGAGCTTAGTACGTGAAACATGAGAGCTTAGTACATTAGCCATGAGAGCTTA |
| | | GTACATTAGCCATGAGGGTTTAGTTCATTAAACATGAGAGCTTAGTACATTAAACATGAGAGCTTAG |
| | | TACATTAAACATGAGAGCTTAGTACATACTATCAACAGGTTGAACTGCTGATCTGTACAGTAGAATT |
| | | GGTAAAGAGAGTTGTGTAAAATATTGAGTTCGCACATCTTGTTGTCTGATTATTGATTTTTGGCGAAA |
| | | CCATTTGATCATATGACAAGATGTGTATCTACCTTAACTTAATGATTTTGATAAAAATCATTA |
| 48. | NP- | CTAGAAGCTGCATGTCTAAGCTAGACCCTTCAGATTAAAAATAACTGAGGTAAGGGCCTGGGTAGG |
| | MHCK7- | GGAGGTGGTGTGAGACGCTCCTGTCTCTCCTCTATCTGCCCATCGGCCCTTTGGGGAGGAGGAATG |
| | hBGi- | TGCCCAAGGACTAAAAAAAGGCCATGGAGCCAGAGGGGCGAGGGCAACAGACCTTTCATGGGCA |
| | EGFP-P2A- | AACCTTGGGGCCCTGCTGTCTAGCATGCCCCACTACGGGTCTAGGCTGCCCATGTAAGGAGGCAAG |
| | Fluc- | GCCTGGGGACACCCGAGATGCCTGGTTATAATTAACCCAGACATGTGGCTGCCCCCCCCCCCCCAA |
| | | CACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCACTCTGGATCCACTGCTTAAAT |
| | | ACGGACGAGGACAGGGCCCTGTCTCCTCAGCTTCAGGCACCACCACTGACCTGGGACAGTGAATC |
| | | GTAAGTACTAGCAGCTACAATCCAGCTACCATTCTGCTTTTATTTTATGGTTGGGATAAGGCTGGATT |
| | | ATTCTGAGTCCAAGCTAGGCCCTTTTGCTAATCATGTTCATACCTCTTATCTTCCTCCCACAGCTCCT |
| | | GGGCAACGTGCTGGTCTGTGTGCTGGCCCATCACTTTGGCAAAGAATTGCGATCGCCACCATGGTG |
| | | AGCAAGGGCGAGGAGCTGTTCACCGGGGTGGTGCCCATCCTGGTCGAGCTGGACGGCGACGTAAA |
| | | CGGCCACAAGTTCAGCGTGTCCGGCGAGGGCGAGGGCGATGCCACCTACGGCAAGCTGACCCTGA |
| | | AGTTCATCTGCACCACCGGCAAGCTGCCCGTGCCCTGGCCCACCCTCGTGACCACCCTGACCTACG |
| | | GCGTGCAGTGCTTCAGCCGCTACCCCGACCACATGAAGCAGCACGACTTCTTCAAGTCCGCCATGC |
| | | CCGAAGGCTACGTCCAGGAGCGCACCATCTTCTTCAAGGACGACGGCAACTACAAGACCCGCGCC |
| | | GAGGTGAAGTTCGAGGGCGACACCCTGGTGAACCGCATCGAGCTGAAGGGCATCGACTTCAAGGA |
| | | GGACGGCAACATCCTGGGGCACAAGCTGGAGTACAACTACAACAGCCACAACGTCTATATCATGGC |
| | | CGACAAGCAGAAGAACGGCATCAAGGTGAACTTCAAGATCCGCCACAACATCGAGGACGGCAGC |
| | | GTGCAGCTCGCCGACCACTACCAGCAGAACACCCCCATCGGCGACGGCCCCGTGCTGCTGCCCGA |
| | | CAACCACTACCTGAGCACCCAGTCCGCCCTGAGCAAAGACCCCAACGAGAAGCGCGATCACATGG |
| | | TCCTGCTGGAGTTCGTGACCGCCGCCGGGATCACTCTCGGCATGGACGAGCTGTACAAGAATCGCG |
| | | CTACTAACTTCAGCCTGCTGAAGCAGGCTGGAGACGTGGAGGAGAACCCTGGACCTATGGAAGAC |
| | | GCCAAAAACATAAAGAAAGGCCCGGCGCCATTCTATCCGCTGGAAGATGGAACCGCTGGAGAGCA |
| | | ACTGCATAAGGCTATGAAGAGATACGCCCTGGTTCCTGGAACAATTGCTTTTACAGATGCACATATC |
| | | GAGGTGGACATCACTTACGCTGAGTACTTCGAAATGTCCGTTCGGTTGGCAGAAGCTATGAAACGA |
| | | TATGGGCTGAATACAAATCACAGAATCGTCGTATGCAGTGAAAACTCTCTTCAATTCTTTATGCCGGT |
| | | GTTGGGCGCGTTATTTATCGGAGTTGCAGTTGCGCCCGCGAACGACATTTATAATGAACGTGAATTG |
| | | CTCAACAGTATGGGCATTTCGCAGCCTACCGTGGTGTTCGTTTCCAAAAAGGGGTTGCAAAAAATT |
| | | TTGAACGTGCAAAAAAAGCTCCCAATCATCCAAAAAATTATTATCATGGATTCTAAAACGGATTACC |
| | | AGGGATTTCAGTCGATGTACACGTTCGTCACATCTCATCTACCTCCCGGTTTTAATGAATACGATTTT |
| | | GTGCCAGAGTCCTTCGATAGGGACAAGACAATTGCACTGATCATGAACTCCTCTGGATCTACTGGTC |
| | | TGCCTAAAGGTGTCGCTCTGCCTCATAGAACTGCCTGCGTGAGATTCTCGCATGCCAGAGATCCTAT |
| | | TTTTGGCAATCAAATCATTCCGGATACTGCGATTTTAAGTGTTGTTCCATTCCATCACGGTTTTGGAA |
| | | TGTTTACTACACTCGGATATTTGATATGTGGATTTCGAGTCGTCTTAATGTATAGATTTGAAGAAGAG |
| | | CTGTTTCTGAGGAGCCTTCAGGATTACAAGATTCAAAGTGCGCTGCTGGTGCCAACCCTATTCTCCT |
| | | TCTTCGCCAAAAGCACTCTGATTGACAAATACGATTTATCTAATTTACACGAAATTGCTTCTGGTGGC |
| | | GCTCCCCTCTCTAAGGAAGTCGGGGAAGCGGTTGCCAAGAGGTTCCATCTGCCAGGTATCAGGCAA |
| | | GGATATGGGCTCACTGAGACTACATCAGCTATTCTGATTACACCCGAGGGGGATGATAAACCGGGCG |
| | | CGGTCGGTAAAGTTGTTCCATTTTTTGAAGCGAAGGTTGTGGATCTGGATACCGGGAAAACGCTGG |
| | | GCGTTAATCAAAGAGGCGAACTGTGTGTGAGAGGTCCTATGATTATGTCCGGTTATGTAAACAATCC |
| | | GGAAGCGACCAACGCCTTGATTGACAAGGATGGATGGCTACATTCTGGAGACATAGCTTACTGGGA |
| | | CGAAGACGAACACTTCTTCATCGTTGACCGCCTGAAGTCTCTGATTAAGTACAAAGGCTATCAGGT |
| | | GGCTCCCGCTGAATTGGAATCCATCTTGCTCCAACACCCCAACATCTTCGACGCAGGTGTCGCAGG |
| | | TCTTCCCGACGATGACGCCGGTGAACTTCCCGCCGCCGTTGTTGTTTTGGAGCACGGAAAGACGAT |
| | | GACGGAAAAAGAGATCGTGGATTACGTCGCCAGTCAAGTAACAACCGCGAAAAAGTTGCGCGGAG |
| | | GAGTTGTGTTTGTGGACGAAGTACCGAAAGGTCTTACCGGAAAACTCGACGCAAGAAAAATCAGA |
| | | GAGATCCTCATAAAGGCCAAGAAGGGCGGAAAGATCGCCGTGTAACTCGAGAATCAACCTCTGGAT |
| | | TACAAAATTTGTGAAAGATTGACTGGTATTCTTAACTATGTTGCTCCTTTTACGCTATGTGGATACGC |
| | | TGCTTTAATGCCTTTGTATCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCGTGGTGTGCACTGTG |
| | | TTTGCTGACGCAACCCCCACTGGTTGGGGCATTGCCACCACCTGTCAGCTCCTTTCCGGGACTTTC |
| | | GCTTTCCCCCTCCCTATTGCCACGGCGGAACTCATCGCCGCCTGCCTTGCCCGCTGCTGGACAGGG |
| | | GCTCGGCTGTTGGGCACTGACAATTCCGTGGGGTGTGCCTTCTAGTTGCCAGCCATCTGTTGTTTGC |
| | | CCCTCCCCCGTGCCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTATGGGAAGATGT |
| | | CTACTGAGCTGTGCGATCCCTGCTGGGGACTTTCCGCTGGGGACTTTCCGCTGGGGACTTTCCGCCT |
| | | TCAGCTAAGGAAGCTACCAATATTTAGAGGTACATTTTGTTCTAGAACAAAATGTACCGGTACATTTT |
| | | GTTCTGGTACATTTTGTTCTATTAAGGACAACGTAAGTATAGCGCATAGACACGTGGCGCCATCGGA |
| | | TCCCGGGCCCGTCGACTTAAGTGGCTTGTTGTCCACAACCATTAAACCTTAAAAGCTTTAAAAGCCT |
| | | TATATATTCTTTTTTTTCTTATAAAACTTAAAACCTTAGAGGCTATTTAAGTTGCTGATTTATATTAATT |
| | | TTATTGTTCAAACATGAGAGCTTAGTACGTGAAACATGAGAGCTTAGTACATTAGCCATGAGAGCTTA |
| | | GTACATTAGCCATGAGGGTTTAGTTCATTAAACATGAGAGCTTAGTACATTAAACATGAGAGCTTAG |
| | | TACATTAAACATGAGAGCTTAGTACATACTATCAACAGGTTGAACTGCTGATCTGTACAGTAGAATT |
| | | GGTAAAGAGAGTTGTGTAAAATATTGAGTTCGCACATCTTGTTGTCTGATTATTGATTTTTGGCGAAA |
| | | CCATTTGATCATATGACAAGATGTGTATCTACCTTAACTTAATGATTTTGATAAAAATCATTAGCGGCC |
| | | GC |
| 49. | WPRE3- | TCTGGATCCACTGCTTAAATACGGACGAGGACAGGGCCCTGTCTCCTCAGCTTCAGGCACCACCAC |
| | DTS | TGACCTGGGACAGTGAATCGTAAGTACTAGCAGCTACAATCCAGCTACCATTCTGCTTTTATTTTATG |
| | pUC57- | GTTGGGATAAGGCTGGATTATTCTGAGTCCAAGCTAGGCCCTTTTGCTAATCATGTTCATACCTCTTA |
| | CBA-hBGi- | TCTTCCTCCCACAGCTCCTGGGCAACGTGCTGGTCTGTGTGCTGGCCCATCACTTTGGCAAAGAATT |
| | DMD-4- | GCGATCGCCACCATGCTTTGGTGGGAAGAAGTCGAGGACTGCTACGAGCGCGAGGACGTGCAGAA |
| | WPRE3- | GAAAACCTTCACCAAATGGGTCAACGCCCAGTTCAGCAAGTTCGGCAAGCAGCACATCGAGAACC |
| | DTS | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACGTGGCGCCATCGGATCCCGGGCCCGTCGACTGCAGAGGCCTGCATGCAAGCTTG |
| | | GCGTAATCATGGTCATAGCTGTTTCCTGTGTGAAATTGTTATCCGCTCACAATTCCACACAACATACG |
| | | AGCCGGAAGCATAAAGTGTAAAGCCTGGGGTGCCTAATGAGTGAGCTAACTCACATTAATTGCGTT |
| | | GCGCTCACTGCCCGCTTTCCAGTCGGGAAACCTGTCGTGCCAGCTGCATTAATGAATCGGCCAACG |
| | | CGCGGGGAGAGGCGGTTTGCGTATTGGGCGCTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAATC |
| | | AGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCA |
| | | AGTCAGAGGTGGCGAAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTCTCCCTTCGGGAAGCG |
| | | TGGCGCTTTCTCATAGCTCACGCTGTAGGTATCTCAGTTCGGTGTAGGTCGTTCGCTCCAAGCTGGG |
| | | CTGTGTGCACGAACCCCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTACACTAGAAGAACAGT |
| | | ATTTGGTATCTGCGCTCTGCTGAAGCCAGTTACCTTCGGAAAAAGAGTTGGTAGCTCTTGATCCGGC |
| | | AAACAAACCACCGCTGGTAGCGGTGGTTTTTTTGTTTGCAAGCAGCAGATTACGCGCAGAAAAAA |
| | | AGGATCTCAAGAAGATCCTTTGATCTTTTCTACGGGGTCTGACGCTCAGTGGAACGAAAACTCACG |
| | | TTAAGGGATTTTGGTCATGAGATTATCAAAAAGGATCTTCACCTAGATCCTTTTAAATTAAAAATGAA |
| | | GTTTTAAATCAATCTAAAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGAGACCCACGCTCACC |
| | | GGCTCCAGATTTATCAGCAATAAACCAGCCAGCCGGAAGGGCCGAGCGCAGAAGTGGTCCTGCAA |
| | | CTTTATCCGCCTCCATCCAGTCTATTAATTGTTGCCGGGAAGCTAGAGTAAGTAGTTCGCCAGTTAAT |
| | | AGTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCGG |
| | | TTAGCTCCTTCGGTCCTCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGCCCGGCGTCAATACGGGAT |
| | | AATACCGCGCCACATAGCAGAACTTTAAAAGTGCTCATCATTGGAAAACGTTCTTCGGGGCGAAAA |
| | | CTCTCAAGGATCTTACCGCTGTTGAGATCCAGTTCGATGTAACCCACTCGTGCACCCAACTGATCTT |
| | | CAGCATCTTTTACTTTCACCAGCGTTTCTGGGTGAGCAAAAACAGGAAGGCAAAATGCCGCAAAA |
| | | AAGGGAATAAGGGCGACACGGAAATGTTGAATACTCATACTCTTCCTTTTTCAATATTATTGAAGCAT |
| | | TTATCAGGGTTATTGTCTCATGAGCGGATACATATTTGAATGTATTTAGAAAAATAAACAAATAGGGG |
| | | TTCCGCGCACATTTCCCCGAAAAGTGCCACCTGACGTCTAAGAAACCATTATTATCATGACATTAAC |
| | | CTATAAAAATAGGCGTATCACGAGGCCCTTTCGTCTCGCGCGTTTCGGTGATGACGGTGAAAACCTC |
| | | TGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGC |
| | | AGATTGTACTGAGAGTGCACCATATGCGGTGTGAAATACCGCACAGATGCGTAAGGAGAAAATACC |
| | | GCATCAGGCGCCATTCGCCATTCAGGCTGCGCAACTGTTGGGAAGGGCGATCGGTGCGGGCCTCTT |
| | | CGCTATTACGCCAGCTGGCGAAAGGGGGATGTGCTGCAAGGCGATTAAGTTGGGTAACGCCAGGGT |
| | | TTTCCCAGTCACGACGTTGTAAAACGACGGCCAGTGAATTCGAGCTCGGTACCTCGCGAATGCATG |
| | | CGGCCGCCGTTACATAACTTACGGTAAATGGCCCGCCTGGCTGACCGCCCAACGACCCCCGCCCAT |
| | | TGACGTCAATAATGACGTATGTTCCCATAGTAACGCCAATAGGGACTTTCCATTGACGTCAATGGGT |
| | | GGAGTATTTACGGTAAACTGCCCACTTGGCAGTACATCAAGTGTATCATATGCCAAGTACGCCCCCT |
| | | ATTGACGTCAATGACGGTAAATGGCCCGCCTGGCATTATGCCCAGTACATGACCTTATGGGACTTTC |
| | | CTACTTGGCAGTACATCTACGTATTAGTCATCGCTATTACCATGGTCGAGGTGAGCCCCACGTTCTGC |
| | | TTCACTCTCCCCATCTCCCCCCCCTCCCCACCCCCAATTTTGTATTTATTTATTTTTTAATTATTTTGTG |
| | | CAGCGATGGGGGCGGGGGGGGGGGGGGCGCGCGCCAGGCGGGGCGGGGCGGGGCGAGGGGCGG |
| | | GGCGGGGCGAGGCGGAGAGGTGCGGCGGCAGCCAATCAGAGCGGCGCGCTCCGAAAGTTTCCTTT |
| | | TATGGCGAGGCGGCGGCGGCGGCGGCCCTATAAAAAGCGAAGCGCGCGGCGGGCGGGAGTC |
| 50. | pUC57- | TCTGGATCCACTGCTTAAATACGGACGAGGACAGGGCCCTGTCTCCTCAGCTTCAGGCACCACCAC |
| | MHCK7- | TGACCTGGGACAGTGAATCGTAAGTACTAGCAGCTACAATCCAGCTACCATTCTGCTTTTATTTTATG |
| | hBGi-DMD- | GTTGGGATAAGGCTGGATTATTCTGAGTCCAAGCTAGGCCCTTTTGCTAATCATGTTCATACCTCTTA |
| | 4-WPRE3- | TCTTCCTCCCACAGCTCCTGGGCAACGTGCTGGTCTGTGTGCTGGCCCATCACTTTGGCAAAGAATT |
| | DTS | GCGATCGCCACCATGCTTTGGTGGGAAGAAGTCGAGGACTGCTACGAGCGCGAGGACGTGCAGAA |
| | | GAAAACCTTCACCAAATGGGTCAACGCCCAGTTCAGCAAGTTCGGCAAGCAGCACATCGAGAACC |
| | | TGTTCAGCGACCTGCAGGATGGCAGAAGGCTGCTGGATCTGCTGGAAGGCCTGACAGGACAGAAG |
| | | CTGCCCAAAGAGAAGGGCAGCACAAGAGTGCACGCCCTGAACAACGTGAACAAGGCCCTGAGAG |
| | | TGCTGCAGAACAACAACGTGGACCTGGTCAACATCGGCAGCACCGACATCGTGGACGGCAACCAC |
| | | AAACTGACCCTGGGCCTGATCTGGAACATCATCCTGCACTGGCAAGTGAAGAACGTGATGAAGAA |
| | | CATCATGGCCGGCCTGCAGCAGACCAACAGCGAGAAGATTCTGCTGAGCTGGGTCCGACAGAGCA |
| | | CCCGGAATTACCCTCAAGTGAACGTGATCAACTTCACCACCTCTTGGAGCGACGGACTGGCCCTGA |
| | | ATGCCCTGATCCACAGCCACAGACCTGACCTGTTCGACTGGAACAGCGTCGTGTGTCAGCAGAGC |
| | | GCCACACAGAGGCTGGAACACGCCTTCAATATCGCCAGATACCAGCTGGGCATCGAGAAACTGCTG |
| | | GACCCCGAGGATGTGGACACCACCTATCCTGACAAGAAATCCATCCTCATGTACATCACCAGCCTGT |
| | | TCCAGGTGCTGCCCCAGCAGGTTTCCATCGAGGCCATTCAAGAGGTCGAGATGCTGCCCAGACCTC |
| | | CTAAAGTGACCAAAGAGGAACACTTCCAGCTGCACCACCAGATGCACTACTCTCAGCAGATCACC |
| | | GTGTCTCTGGCCCAGGGCTACGAGAGAACAAGCAGCCCCAAGCCTCGGTTCAAGAGCTACGCCTAT |
| | | ACACAGGCCGCCTACGTGACCACCAGCGATCCTACAAGAAGCCCATTTCCTAGCCAGCATCTGGAA |
| | | GCCCCTGAGGACAAGAGCTTTGGCAGCAGCCTGATGGAAAGCGAAGTGAACCTGGACCGCTACCA |
| | | GACAGCCCTGGAAGAGGTTCTGAGCTGGCTGCTGTCTGCCGAGGATACACTGCAGGCTCAGGGCG |
| | | AGATCAGCAACGACGTGGAAGTGGTCAAGGACCAGTTTCACACCCACGAGGGCTACATGATGGAC |
| | | CTGACAGCCCACCAGGGCAGAGTGGGCAATATTCTGCAGCTGGGCTCCAAGCTGATCGGCACAGG |
| | | CAAGCTGAGCGAGGACGAAGAGACAGAGGTGCAAGAGCAGATGAACCTGCTGAACAGCAGATGG |
| | | GAGTGTCTGAGAGTGGCCAGCATGGAAAAGCAGAGCAACCTGCACCGGGTGCTGATGGATCTCCA |
| | | GAACCAGAAGCTGAAAGAGCTGAACGACTGGCTGACCAAGACCGAGGAACGGACCCGGAAGATG |
| | | GAAGAGGAACCTCTGGGACCCGACCTGGAAGATCTGAAAAGACAGGTGCAGCAGCATAAGGTGCT |
| | | GCAAGAGGACCTCGAACAAGAGCAAGTGCGCGTGAACAGCCTGACACACATGGTGGTGGTCGTG |
| | | GATGAGAGCAGCGGAGATCATGCCACAGCCGCTCTGGAAGAACAGCTGAAGGTGCTGGGAGACA |
| | | GATGGGCCAATATCTGCCGGTGGACCGAGGATAGATGGGTGCTGCTCCAGGACATCCTGCTGAAGT |
| | | GGCAGCGGCTGACAGAGGAACAGTGCCTGTTTAGCGCCTGGCTGTCCGAGAAAGAGGACGCCGTC |
| | | AACAAGATCCACACCACCGGCTTCAAGGATCAGAATGAGATGCTGAGCAGCCTGCAGAAACTGGC |
| | | CGTGCTGAAGGCTGACCTGGAAAAGAAAAAGCAGTCCATGGGCAAGCTGTACTCCCTGAAGCAGG |
| | | ACCTGCTGAGCACACTGAAGAACAAGTCTGTGACCCAGAAAACCGAGGCCTGGCTGGACAACTTC |
| | | GCCCGGTGTTGGGATAACCTGGTGCAGAAGCTGGAAAAGTCCACCGCTCAGATCTCTCAGGCCGTG |
| | | ACCACAACACAGCCTTCTCTGACCCAGACCACCGTGATGGAAACAGTGACCACAGTGACAACCCG |
| | | CGAGCAGATCCTGGTCAAGCACGCCCAAGAAGAACTGCCTCCTCCACCTCCTCAGAAGAAACGGC |
| | | AGATCACAGTGGACAGCGAGATCCGGAAGAGACTGGACGTGGACATCACCGAGCTGCACAGCTGG |
| | | ATCACCAGATCCGAAGCCGTGCTGCAGTCCCCTGAGTTCGCCATCTTCAGAAAAGAGGGCAACTTC |
| | | TCCGACCTGAAAGAGAAAGTCAACGCCATCGAGAGAGAGAAGGCCGAGAAGTTCCGGAAGCTGC |
| | | AGGACGCCTCTAGATCTGCCCAGGCTCTGGTGGAACAGATGGTCAACGAAGGCGTGAACGCCGAC |
| | | AGCATCAAGCAGGCCTCAGAGCAGCTGAACTCCCGGTGGATCGAGTTCTGTCAGCTCCTGAGCGA |
| | | GAGACTGAACTGGCTGGAATACCAGAACAATATCATTGCCTTCTACAACCAGCTCCAGCAGCTCGA |
| | | ACAGATGACCACCACAGCCGAGAATTGGCTGAAGATCCAGCCTACCACACCTAGCGAGCCCACCG |
| | | CCATTAAGAGCCAGCTGAAAATCTGCAAGGACGAAGTGAATCGGCTGAGCGATCTGCAGCCCCAG |
| | | ATCGAAAGACTGAAAATCCAGTCTATCGCCCTCAAAGAGAAAGGACAGGGCCCCATGTTCCTGGAC |
| | | GCCGATTTTGTGGCCTTTACCAACCACTTCAAACAGGTGTTCTCCGACGTGCAGGCCCGGGAAAAA |
| | | GAGCTGCAGACCATCTTCGACACCCTGCCTCCAATGAGATACCAAGAGACAATGAGCGCCATCCGG |
| | | ACCTGGGTGCAGCAAAGCGAGACAAAGCTGAGCATCCCACAGCTGAGCGTGACCGACTACGAGAT |
| | | CATGGAACAGAGACTGGGCGAACTGCAGGCCCTCCAGTCTAGCCTGCAAGAACAGCAGTCCGGCC |
| | | TGTACTACCTGAGCACAACCGTGAAAGAGATGAGCAAGAAGGCCCCTAGCGAGATCTCCCGGAAG |
| | | TACCAGAGCGAGTTCGAAGAGATCGAAGGCCGGTGGAAGAAGCTGTCTAGCCAGCTGGTTGAGCA |
| | | CTGCCAGAAACTCGAAGAACAAATGAACAAGCTGCGCAAGATCCAGAATCACATCCAGACGCTGA |
| | | AGAAATGGATGGCCGAGGTGGACGTGTTCCTGAAAGAAGAGTGGCCCGCTCTGGGCGACTCCGAG |
| | | ATTCTGAAGAAACAGCTCAAACAGTGCCGGCTGCTGGTGTCCGATATCCAGACAATCCAGCCAAGC |
| | | CTGAACTCCGTGAATGAAGGCGGCCAGAAGATCAAGAACGAGGCCGAGCCTGAGTTTGCCAGCAG |
| | | ACTGGAAACCGAACTGAAAGAACTCAACACCCAGTGGGACCACATGTGCCAGCAAGTGTACGCCC |
| | | GGAAAGAGGCCCTGAAAGGCGGACTCGAAAAGACTGTGTCTCTGCAGAAAGACCTGTCTGAGATG |
| | | CACGAGTGGATGACCCAGGCCGAAGAGGAATACCTGGAACGGGACTTCGAGTACAAGACCCCTGA |
| | | TGAGCTGCAAAAAGCCGTCGAGGAAATGAAGCGGGCCAAAGAAGAGGCCCAGCAGAAAGAAGCC |
| | | AAAGTCAAGCTGCTGACCGAGTCCGTGAATAGCGTTATCGCTCAGGCCCCTCCTGTGGCTCAAGAG |
| | | GCTCTCAAGAAAGAACTGGAAACTCTGACCACCAACTACCAGTGGCTGTGCACCAGACTGAACGG |
| | | CAAGTGCAAGACACTGGAAGAAGTGTGGGCCTGCTGGCACGAACTGCTGTCCTATCTGGAAAAGG |
| | | CCAACAAGTGGCTGAACGAGGTGGAATTCAAGCTGAAAACCACCGAGAACATCCCTGGCGGCGCT |
| | | GAAGAGATTAGCGAGGTGCTGGACAGCCTGGAAAACCTGATGAGACACAGCGAGGACAACCCCA |
| | | ATCAGATCAGAATCCTGGCTCAGACCCTGACCGATGGCGGCGTGATGGACGAGCTGATCAACGAGG |
| | | AACTCGAAACCTTCAACAGCCGGTGGCGGGAACTGCACGAGGAAGCTGTTCGGAGGCAGAAGTT |
| | | GCTGGAACAGTCTATCCAGAGCGCACAAGAAACCGAGAAGTCCCTGCACCTGATCCAAGAGAGCC |
| | | TGACCTTCATCGACAAGCAGCTGGCCGCTTATATCGCCGACAAGGTGGACGCTGCTCAGATGCCCC |
| | | AAGAGGCACAGAAGATTCAGAGCGACCTGACCAGCCACGAGATTAGCCTGGAAGAAATGAAGAA |
| | | GCACAACCAGGGCAAAGAGGCCGCTCAGAGAGTCCTGAGCCAGATCGATGTGGCACAGAAAAAG |
| | | CTCCAGGATGTGTCCATGAAGTTTCGGCTGTTTCAGAAGCCCGCCAACTTCGAGCAGAGACTGCAA |
| | | GAGTCCAAGATGATCCTGGACGAAGTCAAAATGCATCTGCCCGCACTGGAAACAAAGTCCGTGGA |
| | | ACAAGAAGTGGTGCAGTCCCAGCTGAACCACTGCGTGAACCTGTACAAGAGCCTGTCCGAAGTGA |
| | | AGTCTGAGGTGGAAATGGTCATCAAGACCGGCAGGCAGATCGTCCAGAAGAAGCAGACCGAGAAT |
| | | CCCAAAGAACTCGACGAGAGAGTGACCGCTCTGAAGCTGCACTACAATGAGCTGGGCGCCAAAGT |
| | | GACCGAGCGGAAGCAACAGCTTGAGAAGTGCCTGAAACTGTCTCGGAAGATGCGGAAAGAAATG |
| | | AACGTGCTGACAGAGTGGCTGGCCGCCACCGATATGGAACTGACTAAGAGAAGCGCCGTGGAAGG |
| | | CATGCCCAGCAACCTGGATAGTGAAGTGGCCTGGGGCAAAGCCACTCAGAAAGAGATTGAGAAGC |
| | | AGAAGGTCCACCTGAAGTCCATCACCGAAGTGGGCGAAGCCCTGAAAACCGTGCTGGGAAAGAA |
| | | AGAGACACTGGTCGAGGACAAGCTGTCCCTGCTGAATTCCAACTGGATCGCCGTGACCTCCAGAG |
| | | CTGAGGAATGGCTGAATCTGCTGCTTGAGTACCAGAAACACATGGAAACGTTCGACCAGAACGTC |
| | | GACCACATCACAAAGTGGATCATCCAGGCTGATACCCTGCTGGACGAGTCCGAGAAGAAGAAACC |
| | | CCAACAAAAAGAAGATGTGCTGAAGCGGCTGAAAGCCGAGCTGAATGACATCCGGCCTAAGGTGG |
| | | ACAGCACCAGAGATCAGGCAGCCAACCTGATGGCCAACAGAGGCGACCACTGTCGGAAGCTCGTG |
| | | GAACCTCAGATCAGCGAGCTTAACCACAGATTTGCCGCCATCAGCCACCGGATCAAGACAGGCAA |
| | | GGCCAGCATTCCTCTCAAAGAGCTTGAGCAGTTCAACAGCGACATCCAAAAGCTGCTCGAACCCCT |
| | | GGAAGCCGAGATCCAACAGGGCGTCAACCTCAAAGAAGAAGATTTCAACAAGGACATGAACGAG |
| | | GACAATGAGGGCACCGTCAAAGAACTGCTGCAGCGGGGCGATAATCTGCAGCAGCGGATTACCGA |
| | | TGAGCGCAAGCGGGAAGAGATCAAGATTAAGCAGCAGCTGCTGCAGACCAAGCACAACGCCCTGA |
| | | AGGACCTGCGGAGCCAGAGAAGAAAGAAGGCCCTGGAAATCAGCCACCAGTGGTATCAGTACAA |
| | | GCGGCAGGCCGACGACCTGCTGAAGTGCCTGGATGACATCGAGAAGAAGCTGGCCTCTCTGCCCG |
| | | AGCCTAGGGACGAGAGAAAGATCAAAGAGATCGACCGCGAGCTGCAGAAAAAGAAAGAGGAACT |
| | | GAACGCCGTCCGCAGACAGGCCGAAGGACTTTCTGAAGATGGCGCCGCTATGGCCGTGGAACCCA |
| | | CACAGATTCAGCTGAGCAAGCGGTGGCGGGAAATCGAGAGCAAGTTCGCCCAGTTCCGGCGGCTG |
| | | AATTTCGCCCAGATCCACACCGTGCGGGAAGAGACAATGATGGTCATGACAGAGGACATGCCCCTT |
| | | GAGATCAGCTACGTGCCCAGCACCTACCTGACCGAGATCACCCATGTGTCTCAGGCCCTGCTGGAA |
| | | GTGGAACAGCTGCTGAATGCCCCTGACCTGTGCGCCAAGGACTTCGAGGACCTGTTCAAGCAAGA |
| | | GGAAAGCCTGAAGAACATCAAGGACAGCCTGCAGCAGAGCAGCGGCCGGATCGATATCATCCACA |
| | | GCAAGAAAACCGCCGCACTGCAGAGCGCCACACCTGTGGAAAGAGTGAAGCTGCAAGAGGCCCT |
| | | GAGCCAGCTGGATTTCCAGTGGGAGAAAGTGAACAAGATGTACAAGGACCGGCAGGGCAGATTCG |
| | | ACCGCAGCGTTGAAAAGTGGCGGCGGTTCCACTACGACATCAAGATCTTCAACCAGTGGCTGACC |
| | | GAGGCCGAGCAGTTCCTGAGAAAGACACAGATCCCCGAGAACTGGGAGCACGCCAAGTACAAGT |
| | | GGTATCTGAAAGAGCTGCAGGACGGCATCGGCCAGAGACAGACAGTCGTGCGGACACTGAATGCC |
| | | ACCGGCGAGGAAATCATCCAGCAGTCCAGCAAGACCGACGCCAGCATCCTGCAAGAGAAGCTGGG |
| | | CAGCCTGAACCTGCGGTGGCAAGAAGTGTGCAAGCAGCTGAGCGACCGGAAGAAGCGGCTGGAA |
| | | GAACAGAAGAATATCCTGAGCGAGTTCCAGCGGGACCTGAACGAGTTCGTGCTGTGGCTCGAAGA |
| | | GGCCGACAATATCGCCAGCATTCCCCTGGAACCTGGCAAAGAGCAGCAGCTGAAAGAAAAGCTGG |
| | | AACAAGTGAAACTGCTGGTCGAGGAACTGCCCCTGAGACAGGGAATCCTGAAACAGCTGAACGA |
| | | GACAGGCGGCCCTGTGCTGGTGTCTGCCCCTATTTCTCCCGAGGAACAGGACAAGCTGGAAAACA |
| | | AGCTGAAGCAGACCAACCTGCAGTGGATCAAGGTGTCCAGAGCACTGCCTGAGAAGCAGGGCGA |
| | | GATCGAGGCCCAGATCAAGGACCTGGGACAGCTCGAAAAGAAGCTTGAGGACCTCGAAGAACAG |
| | | CTCAACCATCTGCTGCTTTGGCTGAGCCCCATCCGGAACCAGCTGGAAATCTACAACCAGCCTAATC |
| | | AAGAGGGCCCCTTCGACGTGAAAGAAACCGAGATTGCCGTGCAGGCCAAGCAGCCCGATGTGGAA |
| | | GAGATCCTGTCTAAGGGCCAGCACCTGTACAAAGAGAAGCCCGCCACACAGCCCGTGAAGCGGAA |
| | | ACTGGAAGATCTGAGCAGCGAGTGGAAGGCCGTGAACCGGCTGCTGCAAGAACTGAGAGCCAAA |
| | | CAGCCTGATCTGGCCCCTGGCCTGACAACAATTGGCGCCTCTCCTACACAGACCGTGACACTGGTC |
| | | ACACAGCCTGTGGTCACCAAAGAGACAGCCATCAGCAAACTGGAAATGCCCAGCAGTCTGATGCT |
| | | CGAAGTGCCCGCACTGGCCGACTTCAATAGAGCCTGGACCGAGCTGACCGACTGGCTCAGTCTGCT |
| | | GGACCAAGTCATCAAGTCCCAGAGAGTGATGGTCGGAGATCTTGAGGACATCAACGAGATGATCAT |
| | | TAAGCAGAAAGCCACCATGCAGGACCTTGAGCAGAGAAGGCCCCAGCTTGAAGAACTGATCACCG |
| | | CCGCTCAGAACCTGAAAAACAAGACCAGCAATCAAGAAGCCCGGACCATCATCACCGACCGGATC |
| | | GAGAGAATCCAGAACCAGTGGGACGAAGTGCAAGAGCATCTGCAGAACAGACGGCAGCAGCTCA |
| | | ATGAGATGCTGAAGGACAGCACACAGTGGCTGGAAGCCAAAGAAGAAGCCGAGCAGGTTCTCGG |
| | | ACAGGCCAGAGCTAAGCTGGAATCTTGGAAAGAGGGACCCTACACCGTCGACGCCATCCAGAAGA |
| | | AGATCACCGAGACAAAGCAGCTGGCCAAGGATCTGAGACAGTGGCAGACCAATGTGGACGTGGCC |
| | | AACGACCTGGCTCTGAAGCTGCTGAGAGACTACAGCGCCGACGACACCAGAAAGGTGCACATGAT |
| | | CACCGAAAACATCAACGCCTCTTGGCGGAGCATCCACAAGCGGGTGTCAGAGAGAGAAGCCGCTC |
| | | TGGAAGAAACACATAGACTGCTCCAGCAGTTCCCACTCGACCTGGAAAAGTTCCTGGCCTGGCTG |
| | | ACAGAAGCCGAAACCACCGCTAACGTGCTCCAGGATGCCACCAGAAAAGAGCGGCTGTTGGAGG |
| | | ACAGCAAGGGCGTCAAAGAACTCATGAAGCAGTGGCAGGATCTGCAAGGGGAGATTGAAGCCCA |
| | | CACCGACGTGTACCACAACCTGGACGAGAACAGCCAGAAGATCCTGCGGTCCCTGGAAGGCTCTG |
| | | ATGATGCTGTGCTGCTTCAGAGGCGGCTGGACAACATGAACTTCAAGTGGAGCGAGCTGCGGAAG |
| | | AAGTCCCTGAACATCAGAAGCCACCTGGAAGCCTCCAGCGACCAGTGGAAAAGACTGCACCTGTC |
| | | TCTCCAAGAGCTGCTCGTGTGGCTGCAGCTGAAGGATGACGAGCTGAGTAGACAGGCCCCTATCGG |
| | | CGGAGATTTTCCCGCCGTGCAGAAACAGAACGACGTGCACCGGGCCTTCAAGAGAGAGCTGAAAA |
| | | CAAAAGAACCCGTCATCATGAGCACCCTGGAAACCGTGCGGATCTTTCTGACCGAGCAGCCTCTGG |
| | | AAGGACTGGAAAAGCTGTACCAAGAGCCTAGAGAGCTGCCTCCTGAAGAAAGGGCCCAGAACGT |
| | | GACACGGCTGCTTAGAAAGCAGGCCGAGGAAGTGAACACCGAATGGGAGAAGCTGAACCTCCAC |
| | | TCCGCCGACTGGCAGCGGAAGATCGATGAGACACTGGAACGGCTGCGGGAACTGCAAGAAGCCA |
| | | CAGACGAGCTGGACCTGAAACTGCGGCAGGCTGAAGTGATCAAAGGCTCCTGGCAGCCAGTGGGC |
| | | GATCTGCTGATCGATAGCCTGCAGGACCATCTGGAAAAAGTCAAGGCCCTGCGGGGAGAGATCGCC |
| | | CCTCTCAAAGAAAACGTGTCCCACGTGAACGATCTGGCCAGGCAGCTGACAACACTGGGCATCCA |
| | | GCTGAGCCCTTACAACCTGTCTACCTTGGAGGACCTGAATACCCGGTGGAAGCTGCTCCAAGTGGC |
| | | CGTCGAGGATAGAGTGCGGCAGCTGCATGAGGCTCACAGAGATTTTGGACCCGCCAGCCAGCACTT |
| | | CCTGAGCACATCTGTTCAAGGCCCCTGGGAGAGAGCTATCAGCCCTAACAAGGTGCCCTACTACAT |
| | | CAACCACGAGACACAGACCACCTGTTGGGATCACCCCAAGATGACCGAACTGTATCAGTCCCTGGC |
| | | CGACCTGAACAACGTGCGGTTTAGCGCCTACCGGACCGCCATGAAGCTGCGCAGACTGCAGAAAG |
| | | CTCTGTGCCTGGACCTGCTGTCTCTGAGCGCTGCTTGTGATGCCCTGGACCAGCATAACCTCAAGC |
| | | AGAACGACCAGCCTATGGACATCCTGCAGATCATCAACTGCCTGACCACCATCTACGACCGGCTCG |
| | | AACAAGAGCACAACAACCTGGTCAACGTGCCCCTGTGCGTGGACATGTGCCTGAATTGGCTGCTG |
| | | AACGTGTACGACACCGGCAGAACCGGCAGGATCAGAGTGCTGAGCTTCAAGACCGGCATCATCTC |
| | | CCTGTGCAAGGCCCACCTTGAGGATAAGTACCGCTACCTGTTTAAGCAGGTCGCCAGCAGCACCGG |
| | | CTTTTGCGACCAAAGAAGGCTGGGACTGCTGCTCCACGACAGCATTCAGATCCCTAGACAGCTGGG |
| | | CGAAGTGGCCTCTTTCGGCGGCTCTAATATCGAGCCTAGCGTGCGGAGCTGCTTCCAGTTCGCCAA |
| | | CAACAAGCCCGAGATTGAGGCCGCACTGTTCCTGGACTGGATGAGACTGGAACCCCAGAGCATGG |
| | | TCTGGCTGCCTGTGCTGCATAGAGTGGCCGCAGCCGAAACAGCCAAGCACCAGGCCAAGTGCAAC |
| | | ATCTGCAAAGAGTGCCCCATCATCGGCTTCCGGTACAGATCCCTGAAGCACTTCAACTACGATATCT |
| | | GCCAGTCCTGCTTCTTCAGCGGCAGAGTGGCCAAGGGCCACAAGATGCACTACCCCATGGTGGAAT |
| | | ACTGCACCCCTACCACCTCTGGCGAGGACGTGCGGGATTTTGCCAAGGTGCTCAAGAACAAGTTCC |
| | | GGACCAAGCGCTACTTCGCTAAGCACCCCAGAATGGGCTACCTGCCAGTGCAGACAGTGCTTGAG |
| | | GGCGACAACATGGAAACCCCTGTGACTCTGATCAACTTCTGGCCCGTGGATAGCGCCCCTGCTAGT |
| | | TCTCCTCAGCTGTCTCACGACGATACCCACAGCAGAATCGAGCACTACGCCTCCAGACTGGCCGAG |
| | | ATGGAAAACAGCAACGGCAGCTACCTGAATGACAGCATCAGCCCCAACGAGAGCATCGACGACGA |
| | | ACATCTGCTCATTCAGCACTACTGCCAGAGCCTGAATCAGGACAGCCCTCTGTCTCAGCCTAGAAG |
| | | CCCTGCACAGATCCTGATCAGCCTGGAAAGCGAGGAACGCGGCGAGCTGGAAAGAATCCTGGCAG |
| | | ACCTGGAAGAGGAAAACCGGAACCTGCAGGCCGAATACGACAGACTCAAGCAGCAGCACGAGCA |
| | | CAAGGGACTGTCTCCACTGCCTTCTCCACCTGAAATGATGCCCACCTCTCCACAGAGCCCCAGAGA |
| | | TGCCGAGCTGATTGCCGAAGCCAAACTGCTGAGGCAGCACAAAGGCAGACTCGAAGCCAGAATGC |
| | | AGATTCTCGAAGATCACAACAAGCAGCTCGAATCCCAGCTGCACAGACTTAGGCAGCTGCTGGAA |
| | | CAGCCTCAGGCAGAGGCCAAAGTGAATGGCACCACCGTGTCTAGCCCTAGCACAAGCCTGCAGAG |
| | | ATCCGACAGCAGCCAGCCAATGCTTCTGAGAGTCGTGGGCTCCCAGACCAGCGATTCTATGGGCGA |
| | | AGAGGACCTGCTCAGCCCTCCTCAGGATACAAGCACCGGCCTGGAAGAAGTGATGGAACAACTGA |
| | | ACAACAGCTTCCCCAGCAGCAGAGGCAGAAACACCCCTGGCAAGCCCATGCGCGAGGACACCATG |
| | | TGATAACTCGAGAATCAACCTCTGGATTACAAAATTTGTGAAAGATTGACTGGTATTCTTAACTATGT |
| | | TGCTCCTTTTACGCTATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTGTGGCCCGTTGTCA |
| | | GGCAACGTGGCGTGGTGTGCACTGTGTTTGCTGACGCAACCCCCACTGGTTGGGGCATTGCCACCA |
| | | CCTGTCAGCTCCTTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTGGGGTGTGCCTT |
| | | CTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCATGCTGGGGA |
| | | TGCGGTGGGCTCTATGGGAAGATGTCTACTGAGCTGTGCGATCCCTGCTGGGGACTTTCCGCTGGG |
| | | GACTTTCCGCTGGGGACTTTCCGCCTTCAGCTAAGGAAGCTACCAATATTTAGAGGTACATTTTGTT |
| | | CTAGAACAAAATGTACCGGTACATTTTGTTCTGGTACATTTTGTTCTATTAAGGACAACGTAAGTATA |
| | | GCGCATAGACACGTGGCGCCATCGGATCCCGGGCCCGTCGACTGCAGAGGCCTGCATGCAAGCTTG |
| | | GCGTAATCATGGTCATAGCTGTTTCCTGTGTGAAATTGTTATCCGCTCACAATTCCACACAACATACG |
| | | AGCCGGAAGCATAAAGTGTAAAGCCTGGGGTGCCTAATGAGTGAGCTAACTCACATTAATTGCGTT |
| | | GCGCTCACTGCCCGCTTTCCAGTCGGGAAACCTGTCGTGCCAGCTGCATTAATGAATCGGCCAACG |
| | | CGCGGGGAGAGGCGGTTTGCGTATTGGGCGCTCTTCCGCTTCCTCGCTCACTGACTCGCTGCGCTC |
| | | GGTCGTTCGGCTGCGGCGAGCGGTATCAGCTCACTCAAAGGCGGTAATACGGTTATCCACAGAATC |
| | | AGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCA |
| | | AGTCAGAGGTGGCGAAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTCTCCCTTCGGGAAGCG |
| | | TGGCGCTTTCTCATAGCTCACGCTGTAGGTATCTCAGTTCGGTGTAGGTCGTTCGCTCCAAGCTGGG |
| | | CTGTGTGCACGAACCCCCCGTTCAGCCCGACCGCTGCGCCTTATCCGGTAACTATCGTCTTGAGTCC |
| | | AACCCGGTAAGACACGACTTATCGCCACTGGCAGCAGCCACTGGTAACAGGATTAGCAGAGCGAG |
| | | GTATGTAGGCGGTGCTACAGAGTTCTTGAAGTGGTGGCCTAACTACGGCTACACTAGAAGAACAGT |
| | | ATTTGGTATCTGCGCTCTGCTGAAGCCAGTTACCTTCGGAAAAAGAGTTGGTAGCTCTTGATCCGGC |
| | | AAACAAACCACCGCTGGTAGCGGTGGTTTTTTTGTTTGCAAGCAGCAGATTACGCGCAGAAAAAA |
| | | AGGATCTCAAGAAGATCCTTTGATCTTTTCTACGGGGTCTGACGCTCAGTGGAACGAAAACTCACG |
| | | TTAAGGGATTTTGGTCATGAGATTATCAAAAAGGATCTTCACCTAGATCCTTTTAAATTAAAAATGAA |
| | | GTTTTAAATCAATCTAAAGTATATATGAGTAAACTTGGTCTGACAGTTACCAATGCTTAATCAGTGAG |
| | | GCACCTATCTCAGCGATCTGTCTATTTCGTTCATCCATAGTTGCCTGACTCCCCGTCGTGTAGATAAC |
| | | TACGATACGGGAGGGCTTACCATCTGGCCCCAGTGCTGCAATGATACCGCGAGACCCACGCTCACC |
| | | GGCTCCAGATTTATCAGCAATAAACCAGCCAGCCGGAAGGGCCGAGCGCAGAAGTGGTCCTGCAA |
| | | CTTTATCCGCCTCCATCCAGTCTATTAATTGTTGCCGGGAAGCTAGAGTAAGTAGTTCGCCAGTTAAT |
| | | AGTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCGG |
| | | TTAGCTCCTTCGGTCCTCCGATCGTTGTCAGAAGTAAGTTGGCCGCAGTGTTATCACTCATGGTTATG |
| | | GCAGCACTGCATAATTCTCTTACTGTCATGCCATCCGTAAGATGCTTTTCTGTGACTGGTGAGTACTC |
| | | AACCAAGTCATTCTGAGAATAGTGTATGCGGCGACCGAGTTGCTCTTGCCCGGCGTCAATACGGGAT |
| | | AATACCGCGCCACATAGCAGAACTTTAAAAGTGCTCATCATTGGAAAACGTTCTTCGGGGCGAAAA |
| | | CTCTCAAGGATCTTACCGCTGTTGAGATCCAGTTCGATGTAACCCACTCGTGCACCCAACTGATCTT |
| | | CAGCATCTTTTACTTTCACCAGCGTTTCTGGGTGAGCAAAAACAGGAAGGCAAAATGCCGCAAAA |
| | | AAGGGAATAAGGGCGACACGGAAATGTTGAATACTCATACTCTTCCTTTTTCAATATTATTGAAGCAT |
| | | TTATCAGGGTTATTGTCTCATGAGCGGATACATATTTGAATGTATTTAGAAAAATAAACAAATAGGGG |
| | | TTCCGCGCACATTTCCCCGAAAAGTGCCACCTGACGTCTAAGAAACCATTATTATCATGACATTAAC |
| | | CTATAAAAATAGGCGTATCACGAGGCCCTTTCGTCTCGCGCGTTTCGGTGATGACGGTGAAAACCTC |
| | | TGACACATGCAGCTCCCGGAGACGGTCACAGCTTGTCTGTAAGCGGATGCCGGGAGCAGACAAGC |
| | | CCGTCAGGGCGCGTCAGCGGGTGTTGGCGGGTGTCGGGGCTGGCTTAACTATGCGGCATCAGAGC |
| | | AGATTGTACTGAGAGTGCACCATATGCGGTGTGAAATACCGCACAGATGCGTAAGGAGAAAATACC |
| | | GCATCAGGCGCCATTCGCCATTCAGGCTGCGCAACTGTTGGGAAGGGCGATCGGTGCGGGCCTCTT |
| | | CGCTATTACGCCAGCTGGCGAAAGGGGGATGTGCTGCAAGGCGATTAAGTTGGGTAACGCCAGGGT |
| | | TTTCCCAGTCACGACGTTGTAAAACGACGGCCAGTGAATTCGAGCTCGGTACCTCGCGAATGCATC |
| | | TAGAAGCTGCATGTCTAAGCTAGACCCTTCAGATTAAAAATAACTGAGGTAAGGGCCTGGGTAGGG |
| | | GAGGTGGTGTGAGACGCTCCTGTCTCTCCTCTATCTGCCCATCGGCCCTTTGGGGAGGAGGAATGT |
| | | GCCCAAGGACTAAAAAAAGGCCATGGAGCCAGAGGGGCGAGGGCAACAGACCTTTCATGGGCAA |
| | | ACCTTGGGGCCCTGCTGTCTAGCATGCCCCACTACGGGTCTAGGCTGCCCATGTAAGGAGGCAAGG |
| | | CCTGGGGACACCCGAGATGCCTGGTTATAATTAACCCAGACATGTGGCTGCCCCCCCCCCCCCAAC |
| | | ACCTGCTGCCTCTAAAAATAACCCTGTCCCTGGTGGATCCCCTGCATGCGAAGATCTTCGAACAAG |
| | | GCTGTGGGGGACTGAGGGCAGGCTGTAACAGGCTTGGGGGCCAGGGCTTATACGTGCCTGGGACT |
| | | CCCAAAGTATTACTGTTCCATGTTCCCGGCGAAGGGCCAGCTGTCCCCCGCCAGCTAGACTCAGCA |
| | | CTTAGTTTAGGAACCAGTGAGCAAGTCAGCCCTTGGGGCAGCCCATACAAGGCCATGGGGCTGGGC |
| | | AAGCTGCACGCCTGGGTCCGGGGTGGGCACGGTGCCCGGGCAACGAGCTGAAAGCTCATCTGCTC |
| | | TCAGGGGCCCCTCCCTGGGGACAGCCCCTCCTGGCTAGTCACACCCTGTAGGCTCCTCTATATAACC |
| | | CAGGGGCACAGGGGCTGCCCTCATTCTACCACCACCTCCACAGCAC |
| 51. | hBGi intron | TCTGGATCCACTGCTTAAATACGGACGAGGACAGGGCCCTGTCTCCTCAGCTTCAGGCACCACCAC |
| | | TGACCTGGGACAGTGAATCGTAAGTACTAGCAGCTACAATCCAGCTACCATTCTGCTTTTATTTTATG |
| | | GTTGGGATAAGGCTGGATTATTCTGAGTCCAAGCTAGGCCCTTTTGCTAATCATGTTCATACCTCTTA |
| | | TCTTCCTCCCACAGCTCCTGGGCAACGTGCTGGTCTGTGTGCTGGCCCATCACTTTGGCAAAGAATT |
| | | GCGATCGCCACC |

1-19. (canceled)

20. A nucleic acid comprising a nucleic acid sequence having at least 90% sequence identity to SEQ ID NO: 4.

21. The nucleic acid of claim 20, wherein the nucleic acid sequence has at least 91% sequence identity to SEQ ID NO: 4.

22. The nucleic acid of claim 20, wherein the nucleic acid sequence has at least 92% sequence identity to SEQ ID NO: 4.

23. The nucleic acid of claim 20, wherein the nucleic acid sequence has at least 93% sequence identity to SEQ ID NO: 4.

24. The nucleic acid of claim 20, wherein the nucleic acid sequence has at least 94% sequence identity to SEQ ID NO: 4.

25. The nucleic acid of claim 20, wherein the nucleic acid sequence has at least 95% sequence identity to SEQ ID NO: 4.

26. The nucleic acid of claim 20, wherein the nucleic acid sequence has at least 96% sequence identity to SEQ ID NO: 4.

27. The nucleic acid of claim 20, wherein the nucleic acid sequence has at least 97% sequence identity to SEQ ID NO: 4.

28. The nucleic acid of claim 20, wherein the nucleic acid sequence has at least 98% sequence identity to SEQ ID NO: 4.

29. The nucleic acid of claim 20, wherein the nucleic acid sequence has at least 99% sequence identity to SEQ ID NO: 4.

30. The nucleic acid of claim 20, wherein the nucleic acid sequence is a muscle tissue specific promoter sequence.

31. The nucleic acid of claim 20, wherein the nucleic acid sequence enhances expression of a therapeutic transgene in a muscle cell.

32. The nucleic acid of claim 31, wherein the muscle cell comprises a myocyte, a cardiomyocyte, or a smooth muscle cell.

33. The nucleic acid of claim 31, wherein the nucleic acid sequence facilitates expression of a transgene coupled to the promoter for at least 30, 60, 90, 120, 150, 180, 210, 240, 270, or 275 days.

34. The nucleic acid of claim 31, wherein the nucleic acid sequence is comprised within a nucleic acid delivery vector and coupled to a nucleic acid coding sequence encoding a therapeutic transgene, wherein the therapeutic transgene encodes a protein having at least 90% sequence identity to SEQ ID NO: 1.

35. The nucleic acid of claim 1, wherein the nucleic acid is comprised within a kit comprising: (a) the nucleic acid, and (b) a sonoactive agent.

36. The nucleic acid of claim 35, wherein the sonoactive agent comprises lipid-stabilized microstructures.

37. The nucleic acid of claim 35, wherein the sonoactive agent comprises a lipid stabilized shell surrounding a perfluorinated gas core.

38. The nucleic acid of claim 35, wherein the sonoactive agent comprises protein stabilized microstructures.

39. The nucleic acid of claim 35, wherein the sonoactive agent comprises an albumin stabilized shell surrounding a perfluorinated gas core.


SEQUENCE LISTING (51 sequences)

SEQ ID NO: 1 - DNA, 11061 bp, synthetic construct
  source 1..11061: mol_type = other DNA; note = DMD-WT
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggaaaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggagccaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgaaacccttgaaagactccgggaacttcaagaggccacggatgagctggacctcaagctgcgccaagctgaggtgatcaagggatcctggcagcccgtgggcgatctcctcattgactctctccaagatcacctggagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtagtaa

SEQ ID NO: 2 - DNA, 11061 bp, synthetic construct
  source 1..11061: mol_type = other DNA; note = DMD-3
atgctgtggtgggaagaggtggaggactgttacgagagagaggacgtgcagaagaagacattcaccaagtgggtgaacgctcagttcagcaagttcggcaagcagcacatcgagaacctgtttagcgatctacaagatggcagaagactgctggacctgctggagggcctgaccgggcagaagctgcccaaagaaaaggggtctacaagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaagtgaagaacgtgatgaagaacatcatggccggcctgcagcagaccaacagcgagaagatcctgctgagctgggtgagacagagcacaagaaactacccccaagtgaacgtgatcaacttcaccacaagctggagcgacggcctggccctgaacgccctgatccacagccacagacccgacctgttcgactggaacagcgtggtgtgtcagcagagcgccacacagagactggagcacgccttcaacatcgctagatatcagctgggcatcgagaagttactggaccccgaagatgtggacaccacctaccccgacaagaagagcatcctgatgtacatcacaagcctgttccaagtgctgcctcagcaagtgagcatcgaggccatccaagaagtcgagatgctgcctagaccccccaaggtgaccaaggaggagcactttcagctgcaccatcagatgcactactctcagcagatcacagtcagcttggcacaaggctacgagagaacaagcagccccaagcctagattcaagagctacgcctacacccaagccgcctacgtgaccacaagtgaccccacaagaagccccttcccttctcagcacctggaggcccccgaggacaagagcttcggcagcagcctgatggagagcgaggtgaacctggacagatatcagaccgcgttagaagaagtgttgagctggctgctgagcgccgaggacaccctgcaagcccaaggcgagatcagcaacgatgtggaggtggtgaaggatcagttccacacccacgagggctacatgatggacctgaccgcccaccaaggcagagtgggcaacatcctgcagctgggcagcaagctgatcggcaccggcaagctgagcgaggacgaggaaaccgaggttcaagagcaaatgaacctgctaaacagcagatgggagtgcctgagagtggctagcatggaaaagcagagtaacctgcacagagtgctgatggacctgcagaatcagaaattgaaagaacttaatgattggctgaccaagaccgaggagagaacaagaaagatggaggaggagcccctgggccccgacctggaggacctgaagagacaagtgcagcagcacaaggtgctgcaagaggacctggagcaagagcaagtgagagtgaactctctgacacacatggtggtggtagtggacgagagcagcggcgatcacgctaccgcggcgctggaagagcagctgaaagtgctgggcgacagatgggccaacatctgcagatggaccgaggacagatgggtgctgctgcaagacatcctgctgaagtggcagagactgaccgaggagcagtgcctgttcagcgcctggctgagcgagaaggaagacgctgtgaacaagatccacaccaccggcttcaaggatcagaacgagatgctgagctccctccaaaagctcgcagtgctgaaggccgacctggaaaagaaaaaacagtcgatgggcaagctgtacagcctgaagcaagacctgctgagcaccctgaagaacaagagcgtgacacagaagaccgaggcctggctggacaacttcgctagatgctgggacaacctggtacagaagttagagaagtctaccgctcagatcagccaagccgtaaccaccacacaacctagcctgactcagaccaccgtgatggaaaccgtgaccaccgtgacaacccgtgagcagatcctggtgaagcacgcccaagaggagctgcccccccccccccctcagaagaagagacagatcaccgtggactcagagattcgcaagagactggacgtggacatcaccgagctgcacagctggatcacaagaagcgaggccgtgctgcagagccctgagttcgcgatctttagaaaggagggcaacttcagcgacttgaaggagaaagtgaacgccatcgagagagagaaggccgagaagttcagaaagttgcaagacgcctcaagaagcgcccaagccctggtggagcagatggttaacgaaggcgtgaatgccgacagcatcaagcaagctagcgagcagctgaatagccggtggattgaattctgtcagctgctgagcgagagattaaactggctggagtatcagaacaacatcatcgccttctacaatcagctgcagcaactagaacaaatgaccaccaccgccgagaactggctaaagattcagcccaccacccctagcgagcccaccgccatcaaatctcagctgaaaatttgcaaggacgaagtgaacagactgtcggacctgcagcctcaaatcgaaagactgaaaattcagagcatcgcacttaaggagaagggccaaggacccatgttcctggacgccgacttcgtggccttcaccaaccacttcaagcaagtgttcagcgacgtgcaagctagagagaaggagctgcagaccatcttcgacaccctgccccccatgagataccaagaaaccatgagcgccatcagaacctgggtgcagcagagcgaaaccaagctgagcatccctcagctgagcgtgaccgactacgagatcatggagcaaagactgggagagctgcaagccctgcagagcagcctgcaagagcagcagagcggcctgtactacctgagcaccaccgtgaaggagatgtcgaagaaagccccgtcggagatcagcagaaagtatcagagcgagttcgaggagatcgagggcagatggaagaagctgagcagccaactggtggaacactgtcagaaattagaagaacagatgaacaagctgagaaagattcagaaccacattcagaccctgaagaagtggatggccgaggtggacgtgttcctgaaagaggagtggcccgccctgggcgattccgaaatcttaaaaaagcagctgaagcagtgcagactgctggtgtcagatatccaaaccattcaaccaagcctgaacagcgtgaacgaaggcgggcagaagatcaagaacgaggccgagcccgagttcgctagcagactggaaaccgagctgaaggagctgaatacacagtgggaccacatgtgtcagcaagtgtacgctagaaaggaagcgctgaagggcgggctggagaagaccgttagcctgcaaaaggatctgagcgagatgcacgagtggatgacccaagccgaggaggagtacctggagagagacttcgagtacaagacccccgacgagctgcagaaggccgtcgaggaaatgaagagagcgaaggaggaggctcagcagaaggaagccaaggtgaagctgctgactgaatctgtcaacagtgtgatagctcaagcaccccccgttgcccaagaggcactaaagaaggagctggaaactctgaccaccaactatcagtggctgtgcacaagactgaacggcaagtgcaagaccctggaggaagtgtgggcctgctggcacgagctgctgagctacctggagaaggccaacaagtggctgaacgaggtggagttcaagctgaagaccaccgagaacatccccggcggcgccgaggagatcagcgaggtgctggacagcctggagaacctgatgagacacagcgaggacaaccccaatcagatcagaatcctggctcagaccctgaccgacggcggcgtgatggacgagctgatcaacgaggagctggaaacctttaactctagatggcgagaactgcacgaggaggccgtgagaagacagaagctgctggagcaaagcattcagagcgcccaagaaaccgagaagagcctgcacctgatccaagagagcctgaccttcatcgacaagcagctggccgcctacatcgccgacaaggtggacgccgctcagatgccacaagaggctcagaagattcagtccgatctgacaagccacgagatcagcctggaggagatgaaaaaacacaaccaaggcaaggaggccgctcagagagtgctgtctcagatcgacgtggctcagaagaagctgcaagacgtgagcatgaagttcagactgtttcagaagcccgccaacttcgagcagcgactgcaagagagcaagatgatcctggacgaggtgaagatgcacctgcccgccctggaaaccaagagcgtggagcaagaggtggtgcagtctcagttgaaccactgcgtgaacctgtacaagagcctgtcggaggtcaagagtgaggtcgagatggtgatcaaaactgggagacagatcgtgcaaaaaaagcagaccgagaaccccaaggagctggacgagcgtgttaccgccctgaagctgcactacaacgagctgggcgccaaggtgaccgagagaaagcagcagctggagaagtgcctgaagctgagcagaaagatgagaaaggagatgaacgtgctgaccgagtggctggccgccaccgacatggagctgaccaagagaagcgccgtggagggcatgcctagcaacctggacagcgaggtggcctggggcaaggccacacagaaggagatcgagaagcagaaggtgcacctgaagagcatcaccgaggtgggcgaggccctgaagaccgtgctgggcaagaaggaaaccctggtggaggacaagctgagcctgctgaactcgaactggatcgccgtgacaagcagagccgaggagtggctgaacctgctgttagagtaccaaaaacacatggaaaccttcgatcagaacgtggaccacatcaccaagtggatcatccaagccgacaccctgctggacgaaagcgaaaagaagaagccgcaacaaaaggaggatgtgctgaagagactgaaggccgaactgaacgatatcagacccaaggtggacagcacacgggaccaagccgccaacctgatggccaacagaggcgaccactgcagaaaactggtggagcctcagatcagcgagctgaaccacagattcgccgccatcagccacagaatcaaaaccggcaaagcgagcatccccttgaaggaactggagcaatttaacagcgacatccaaaaattgctcgaaccactggaggccgagattcagcaaggcgtgaacctgaaggaagaggacttcaacaaggacatgaacgaggacaacgagggcaccgtgaaagaactgctgcagagaggcgacaacctgcagcagagaatcaccgacgagagaaagagagaggagatcaagatcaagcagcagctgctgcagactaagcacaacgccctgaaggatctgagatctcagaggagaaaaaaggctctggaaatttcacatcagtggtatcagtacaagagacaagcagacgacctgctgaagtgcctggacgacatcgagaagaagttagctagcctgcccgagcctagagacgagagaaagatcaaggagatcgacagagagttacaaaagaagaaggaggagctgaatgcggtaagaagacaagcggaaggcctgtccgaggacggcgcagccatggccgtggagcccacacagattcagctcagcaagagatggagagagatcgagagcaagttcgctcagttcagaagactgaacttcgctcagatccacaccgtgagagaggaaaccatgatggtgatgaccgaggacatgcccctggagatctcatacgttcctagcacctacctgaccgagatcacccacgtgagccaagccctgctggaggtggagcagctgctgaacgcccccgacctgtgcgccaaggacttcgaggacctgttcaaacaagaggagagcctgaagaacatcaaggacagcctgcagcaaagtagcggcagaatcgacatcatccacagcaagaagaccgccgccctgcagagcgccacccccgtggagagagtgaagcttcaagaggccctttcgcagctggattttcagtgggagaaggtgaacaaaatgtacaaggacagacaaggcagattcgacagaagcgtggagaagtggagaagattccactacgacatcaagatcttcaatcagtggctgaccgaggcagagcagttcctgagaaagacacagatccccgagaactgggagcacgccaagtacaagtggtacctgaaggagctgcaagacggcatcgggcagagacagaccgtggtgagaaccctgaacgccaccggcgaggagatcatccaacaaagctccaaaaccgacgctagcatcctgcaagagaaactgggcagcctgaacctgagatggcaagaggtgtgcaagcagctgagcgaccgcaaaaaacggttagaggagcagaagaacatcctgagcgagtttcagcgggacctgaacgagttcgtgctgtggctggaggaggccgataacatcgctagcatccccctggagcccggcaaggagcagcagttaaaagaaaaacttgagcaagtgaagctgctggtggaggagctgcccctgagacaaggcatcctgaagcagctgaacgagactggcggccccgtgctggtgagcgcccccatcagccccgaggagcaagacaagctggagaacaagctgaagcagaccaacctgcagtggatcaaggtgagcagagccctgcccgagaagcaaggcgaaatcgaggcccaaataaaggacctggggcagctggagaagaaactcgaagacttggaagaacaactcaaccacttgctgctgtggctgagccccatcagaaatcagctggagatctacaatcagcccaaccaagaggggccattcgacgtgaaggaaaccgagatcgccgtgcaagccaagcagcccgatgtggaggagatcctgagcaaggggcagcacctgtacaaggagaagcccgccacacagcccgtcaagcgtaaactagaggatctttcctctgagtggaaggccgtgaaccgtctgctacaagagctgcgggctaagcaacccgacttagcgcccggcctgaccaccatcggtgcaagccccacgcagaccgtgaccctggtgacccaacccgtggtgaccaaggaaaccgccatcagcaagctggagatgcctagcagcctgatgctggaggtgcccgccctggccgacttcaacagagcctggaccgagctgaccgactggctgagcctgctggaccaagtgatcaagtctcagagagtgatggtgggcgatctggaggacatcaatgagatgatcatcaagcagaaggccaccatgcaagacctggagcagagaagacctcagctggaggagctgatcaccgccgctcagaacctgaaaaacaagacgagcaaccaagaggctagaaccatcatcaccgacagaatcgagagaattcagaatcagtgggacgaggtgcaagagcacctgcagaacagaagacagcaactaaacgaaatgctcaaggacagcacacagtggctggaggccaaggaggaggcggagcaagtgttgggccaagctagagccaagctggagagctggaaggagggaccctacaccgtggacgccattcagaagaagatcaccgaaaccaagcagctggccaaggacctgcgacagtggcagaccaacgtggacgtggccaacgatcttgccctgaagctgctgcgcgactatagcgccgacgacacaagaaaggtgcacatgatcaccgagaacatcaacgctagctggcgtagcatccacaagagagtgagcgagagagaggcggctttggaggaaacccatagactgctgcagcagtttcccctggacctggagaagttcctggcctggctgaccgaagccgaaacaaccgccaacgtgctgcaagacgccacaagaaaggagagactgctggaggacagcaagggcgtgaaggagctgatgaagcagtggcaagacctgcaaggcgagatcgaagcgcacaccgacgtgtaccacaacctggacgagaactctcagaagatcctgagaagcctggagggcagcgacgacgccgtgctgttacagagaaggctggacaacatgaacttcaagtggagcgagctgagaaagaagagcctgaacatcagaagccacctggaggctagcagcgatcagtggaagagactgcacctgagcctgcaagagctgctggtctggcttcagttgaaggacgatgagctgagcagacaagcccccatcggcggcgacttccccgccgtgcagaagcagaacgacgtgcatagagccttcaagagagagctgaagaccaaggagcccgtgatcatgagcaccctggaaacggtgagaatcttcctgaccgagcagcccctggagggcctggagaagctgtaccaagagcctagagagctgccccccgaggagagagctcagaacgtgacaagactgctgagaaagcaagccgaagaggtgaacaccgagtgggagaagctgaacctgcacagcgccgactggcagagaaagatcgacgaaaccctggagagactgagagaattgcaagaggcgaccgacgagctggacctgaagctgagacaagccgaggtcatcaagggcagctggcagcccgtgggcgatctattaattgactctctacaagaccacctggagaaagtgaaggccctgagaggcgagatcgcccccctgaaggagaacgtgagccacgtgaatgatctggcacgtcagctgaccaccctgggcatacaactgtccccctataacctgagcacactggaggatttgaatactagatggaagctgctgcaagtggccgtggaggacagagtaagacaattacatgaggcccacagagacttcggccccgcttctcagcacttcctgagcacaagcgtgcaaggcccctgggagagagccatttcccccaacaaggtcccctactacatcaaccacgagacacagaccacctgctgggaccaccccaagatgaccgagctgtatcagagcctcgccgatctgaacaatgtgagattcagcgcctacagaaccgccatgaagctgagaagactgcagaaggccctgtgcctggacttattaagcctgagcgccgcctgcgacgccctggatcagcacaacctgaagcaaaacgaccaacccatggacatcctgcagatcatcaactgcctcaccaccatatacgacagactggaacaagagcacaacaacctggtgaacgtgcccctgtgcgtggacatgtgcctgaactggctgctgaacgtgtacgacaccggcagaaccggcagaatcagagtgctgagcttcaagaccggcatcatcagcctgtgcaaggcccacctggaggacaaatacagataccttttcaaacaagtggctagcagcaccggcttctgcgaccaaagaagactgggtctgctgctgcacgacagcattcagatccctagacagctgggcgaggtggctagcttcggcggcagcaacatcgagcctagcgtgagaagctgctttcagttcgccaacaacaagcccgagatagaggcagccctcttcttagactggatgagactggagcctcagagcatggtatggctgccggtcctgcacagagtggccgccgccgaaaccgccaagcaccaagccaagtgcaacatctgcaaggagtgccccatcatcggcttcagatacagaagcctgaagcacttcaactacgacatctgtcagagctgcttcttcagcggcagagtggccaagggccacaagatgcactaccccatggtggagtactgcacccccaccacaagcggcgaggacgtgagagacttcgccaaggtgttgaagaataagtttagaaccaagagatacttcgccaagcaccctagaatgggctacctgcccgtgcagaccgtgctggagggcgacaacatggaaacccccgtgaccctgatcaacttctggcccgtggacagcgcccccgctagcagccctcagctgagccacgacgacacccacagcagaatcgagcactacgctagcagactggccgagatggagaacagcaacggcagctacctgaacgacagcatcagcccgaatgagagcatcgacgacgagcacctgctgattcagcactactgtcagagcctgaaccaagacagccccctgtctcagcctagaagccccgctcagatcctgatcagcctggagagcgaggagagaggcgagctggagagaatcctagcagatctggaggaagagaacagaaacctgcaagccgagtatgaccgactgaaacagcaacatgagcacaaaggcctgagcccactgccctccccccccgagatgatgcccacaagccctcagagccctagagacgccgagctgatcgctgaagcgaagttacttagacaacacaagggcagactggaggctagaatgcagatcctggaggaccacaacaagcagctggagtctcagctgcacagactgaggcagctgctggaacagccccaagccgaggccaaagtgaacggcaccaccgtgagcagtcctagcacaagcctgcagagaagcgacagctctcagcccatgctgctgagagtggtgggctctcagacaagcgacagcatgggcgaggaggatctgctgagtccgccccaagacacaagcaccggcctggaagaggtgatggagcaactgaacaatagcttccctagcagcagaggcagaaacacccccggcaagcccatgagagaggacaccatgtgatga

SEQ ID NO: 3 - DNA, 11061 bp, synthetic construct
  source 1..11061: mol_type = other DNA; note = DMD-4
atgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataa

SEQ ID NO: 4 - DNA, 1051 bp, synthetic construct
  source 1..1051: mol_type = other DNA; note = MHCK Promoter
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 5 - DNA, 855 bp, synthetic construct
  source 1..855: mol_type = other DNA; note = MHCK Promoter SF
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatc

SEQ ID NO: 6 - DNA, 12889 bp, synthetic construct
  source 1..12889: mol_type = other DNA; note = MHCK7-hBGi-DMD-3-WPRE3-DTS
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggactgttacgagagagaggacgtgcagaagaagacattcaccaagtgggtgaacgctcagttcagcaagttcggcaagcagcacatcgagaacctgtttagcgatctacaagatggcagaagactgctggacctgctggagggcctgaccgggcagaagctgcccaaagaaaaggggtctacaagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaagtgaagaacgtgatgaagaacatcatggccggcctgcagcagaccaacagcgagaagatcctgctgagctgggtgagacagagcacaagaaactacccccaagtgaacgtgatcaacttcaccacaagctggagcgacggcctggccctgaacgccctgatccacagccacagacccgacctgttcgactggaacagcgtggtgtgtcagcagagcgccacacagagactggagcacgccttcaacatcgctagatatcagctgggcatcgagaagttactggaccccgaagatgtggacaccacctaccccgacaagaagagcatcctgatgtacatcacaagcctgttccaagtgctgcctcagcaagtgagcatcgaggccatccaagaagtcgagatgctgcctagaccccccaaggtgaccaaggaggagcactttcagctgcaccatcagatgcactactctcagcagatcacagtcagcttggcacaaggctacgagagaacaagcagccccaagcctagattcaagagctacgcctacacccaagccgcctacgtgaccacaagtgaccccacaagaagccccttcccttctcagcacctggaggcccccgaggacaagagcttcggcagcagcctgatggagagcgaggtgaacctggacagatatcagaccgcgttagaagaagtgttgagctggctgctgagcgccgaggacaccctgcaagcccaaggcgagatcagcaacgatgtggaggtggtgaaggatcagttccacacccacgagggctacatgatggacctgaccgcccaccaaggcagagtgggcaacatcctgcagctgggcagcaagctgatcggcaccggcaagctgagcgaggacgaggaaaccgaggttcaagagcaaatgaacctgctaaacagcagatgggagtgcctgagagtggctagcatggaaaagcagagtaacctgcacagagtgctgatggacctgcagaatcagaaattgaaagaacttaatgattggctgaccaagaccgaggagagaacaagaaagatggaggaggagcccctgggccccgacctggaggacctgaagagacaagtgcagcagcacaaggtgctgcaagaggacctggagcaagagcaagtgagagtgaactctctgacacacatggtggtggtagtggacgagagcagcggcgatcacgctaccgcggcgctggaagagcagctgaaagtgctgggcgacagatgggccaacatctgcagatggaccgaggacagatgggtgctgctgcaagacatcctgctgaagtggcagagactgaccgaggagcagtgcctgttcagcgcctggctgagcgagaaggaagacgctgtgaacaagatccacaccaccggcttcaaggatcagaacgagatgctgagctccctccaaaagctcgcagtgctgaaggccgacctggaaaagaaaaaacagtcgatgggcaagctgtacagcctgaagcaagacctgctgagcaccctgaagaacaagagcgtgacacagaagaccgaggcctggctggacaacttcgctagatgctgggacaacctggtacagaagttagagaagtctaccgctcagatcagccaagccgtaaccaccacacaacctagcctgactcagaccaccgtgatggaaaccgtgaccaccgtgacaacccgtgagcagatcctggtgaagcacgcccaagaggagctgcccccccccccccctcagaagaagagacagatcaccgtggactcagagattcgcaagagactggacgtggacatcaccgagctgcacagctggatcacaagaagcgaggccgtgctgcagagccctgagttcgcgatctttagaaaggagggcaacttcagcgacttgaaggagaaagtgaacgccatcgagagagagaaggccgagaagttcagaaagttgcaagacgcctcaagaagcgcccaagccctggtggagcagatggttaacgaaggcgtgaatgccgacagcatcaagcaagctagcgagcagctgaatagccggtggattgaattctgtcagctgctgagcgagagattaaactggctggagtatcagaacaacatcatcgccttctacaatcagctgcagcaactagaacaaatgaccaccaccgccgagaactggctaaagattcagcccaccacccctagcgagcccaccgccatcaaatctcagctgaaaatttgcaaggacgaagtgaacagactgtcggacctgcagcctcaaatcgaaagactgaaaattcagagcatcgcacttaaggagaagggccaaggacccatgttcctggacgccgacttcgtggccttcaccaaccacttcaagcaagtgttcagcgacgtgcaagctagagagaaggagctgcagaccatcttcgacaccctgccccccatgagataccaagaaaccatgagcgccatcagaacctgggtgcagcagagcgaaaccaagctgagcatccctcagctgagcgtgaccgactacgagatcatggagcaaagactgggagagctgcaagccctgcagagcagcctgcaagagcagcagagcggcctgtactacctgagcaccaccgtgaaggagatgtcgaagaaagccccgtcggagatcagcagaaagtatcagagcgagttcgaggagatcgagggcagatggaagaagctgagcagccaactggtggaacactgtcagaaattagaagaacagatgaacaagctgagaaagattcagaaccacattcagaccctgaagaagtggatggccgaggtggacgtgttcctgaaagaggagtggcccgccctgggcgattccgaaatcttaaaaaagcagctgaagcagtgcagactgctggtgtcagatatccaaaccattcaaccaagcctgaacagcgtgaacgaaggcgggcagaagatcaagaacgaggccgagcccgagttcgctagcagactggaaaccgagctgaaggagctgaatacacagtgggaccacatgtgtcagcaagtgtacgctagaaaggaagcgctgaagggcgggctggagaagaccgttagcctgcaaaaggatctgagcgagatgcacgagtggatgacccaagccgaggaggagtacctggagagagacttcgagtacaagacccccgacgagctgcagaaggccgtcgaggaaatgaagagagcgaaggaggaggctcagcagaaggaagccaaggtgaagctgctgactgaatctgtcaacagtgtgatagctcaagcaccccccgttgcccaagaggcactaaagaaggagctggaaactctgaccaccaactatcagtggctgtgcacaagactgaacggcaagtgcaagaccctggaggaagtgtgggcctgctggcacgagctgctgagctacctggagaaggccaacaagtggctgaacgaggtggagttcaagctgaagaccaccgagaacatccccggcggcgccgaggagatcagcgaggtgctggacagcctggagaacctgatgagacacagcgaggacaaccccaatcagatcagaatcctggctcagaccctgaccgacggcggcgtgatggacgagctgatcaacgaggagctggaaacctttaactctagatggcgagaactgcacgaggaggccgtgagaagacagaagctgctggagcaaagcattcagagcgcccaagaaaccgagaagagcctgcacctgatccaagagagcctgaccttcatcgacaagcagctggccgcctacatcgccgacaaggtggacgccgctcagatgccacaagaggctcagaagattcagtccgatctgacaagccacgagatcagcctggaggagatgaaaaaacacaaccaaggcaaggaggccgctcagagagtgctgtctcagatcgacgtggctcagaagaagctgcaagacgtgagcatgaagttcagactgtttcagaagcccgccaacttcgagcagcgactgcaagagagcaagatgatcctggacgaggtgaagatgcacctgcccgccctggaaaccaagagcgtggagcaagaggtggtgcagtctcagttgaaccactgcgtgaacctgtacaagagcctgtcggaggtcaagagtgaggtcgagatggtgatcaaaactgggagacagatcgtgcaaaaaaagcagaccgagaaccccaaggagctggacgagcgtgttaccgccctgaagctgcactacaacgagctgggcgccaaggtgaccgagagaaagcagcagctggagaagtgcctgaagctgagcagaaagatgagaaaggagatgaacgtgctgaccgagtggctggccgccaccgacatggagctgaccaagagaagcgccgtggagggcatgcctagcaacctggacagcgaggtggcctggggcaaggccacacagaaggagatcgagaagcagaaggtgcacctgaagagcatcaccgaggtgggcgaggccctgaagaccgtgctgggcaagaaggaaaccctggtggaggacaagctgagcctgctgaactcgaactggatcgccgtgacaagcagagccgaggagtggctgaacctgctgttagagtaccaaaaacacatggaaaccttcgatcagaacgtggaccacatcaccaagtggatcatccaagccgacaccctgctggacgaaagcgaaaagaagaagccgcaacaaaaggaggatgtgctgaagagactgaaggccgaactgaacgatatcagacccaaggtggacagcacacgggaccaagccgccaacctgatggccaacagaggcgaccactgcagaaaactggtggagcctcagatcagcgagctgaaccacagattcgccgccatcagccacagaatcaaaaccggcaaagcgagcatccccttgaaggaactggagcaatttaacagcgacatccaaaaattgctcgaaccactggaggccgagattcagcaaggcgtgaacctgaaggaagaggacttcaacaaggacatgaacgaggacaacgagggcaccgtgaaagaactgctgcagagaggcgacaacctgcagcagagaatcaccgacgagagaaagagagaggagatcaagatcaagcagcagctgctgcagactaagcacaacgccctgaaggatctgagatctcagaggagaaaaaaggctctggaaatttcacatcagtggtatcagtacaagagacaagcagacgacctgctgaagtgcctggacgacatcgagaagaagttagctagcctgcccgagcctagagacgagagaaagatcaaggagatcgacagagagttacaaaagaagaaggaggagctgaatgcggtaagaagacaagcggaaggcctgtccgaggacggcgcagccatggccgtggagcccacacagattcagctcagcaagagatggagagagatcgagagcaagttcgctcagttcagaagactgaacttcgctcagatccacaccgtgagagaggaaaccatgatggtgatgaccgaggacatgcccctggagatctcatacgttcctagcacctacctgaccgagatcacccacgtgagccaagccctgctggaggtggagcagctgctgaacgcccccgacctgtgcgccaaggacttcgaggacctgttcaaacaagaggagagcctgaagaacatcaaggacagcctgcagcaaagtagcggcagaatcgacatcatccacagcaagaagaccgccgccctgcagagcgccacccccgtggagagagtgaagcttcaagaggccctttcgcagctggattttcagtgggagaaggtgaacaaaatgtacaaggacagacaaggcagattcgacagaagcgtggagaagtggagaagattccactacgacatcaagatcttcaatcagtggctgaccgaggcagagcagttcctgagaaagacacagatccccgagaactgggagcacgccaagtacaagtggtacctgaaggagctgcaagacggcatcgggcagagacagaccgtggtgagaaccctgaacgccaccggcgaggagatcatccaacaaagctccaaaaccgacgctagcatcctgcaagagaaactgggcagcctgaacctgagatggcaagaggtgtgcaagcagctgagcgaccgcaaaaaacggttagaggagcagaagaacatcctgagcgagtttcagcgggacctgaacgagttcgtgctgtggctggaggaggccgataacatcgctagcatccccctggagcccggcaaggagcagcagttaaaagaaaaacttgagcaagtgaagctgctggtggaggagctgcccctgagacaaggcatcctgaagcagctgaacgagactggcggccccgtgctggtgagcgcccccatcagccccgaggagcaagacaagctggagaacaagctgaagcagaccaacctgcagtggatcaaggtgagcagagccctgcccgagaagcaaggcgaaatcgaggcccaaataaaggacctggggcagctggagaagaaactcgaagacttggaagaacaactcaaccacttgctgctgtggctgagccccatcagaaatcagctggagatctacaatcagcccaaccaagaggggccattcgacgtgaaggaaaccgagatcgccgtgcaagccaagcagcccgatgtggaggagatcctgagcaaggggcagcacctgtacaaggagaagcccgccacacagcccgtcaagcgtaaactagaggatctttcctctgagtggaaggccgtgaaccgtctgctacaagagctgcgggctaagcaacccgacttagcgcccggcctgaccaccatcggtgcaagccccacgcagaccgtgaccctggtgacccaacccgtggtgaccaaggaaaccgccatcagcaagctggagatgcctagcagcctgatgctggaggtgcccgccctggccgacttcaacagagcctggaccgagctgaccgactggctgagcctgctggaccaagtgatcaagtctcagagagtgatggtgggcgatctggaggacatcaatgagatgatcatcaagcagaaggccaccatgcaagacctggagcagagaagacctcagctggaggagctgatcaccgccgctcagaacctgaaaaacaagacgagcaaccaagaggctagaaccatcatcaccgacagaatcgagagaattcagaatcagtgggacgaggtgcaagagcacctgcagaacagaagacagcaactaaacgaaatgctcaaggacagcacacagtggctggaggccaaggaggaggcggagcaagtgttgggccaagctagagccaagctggagagctggaaggagggaccctacaccgtggacgccattcagaagaagatcaccgaaaccaagcagctggccaaggacctgcgacagtggcagaccaacgtggacgtggccaacgatcttgccctgaagctgctgcgcgactatagcgccgacgacacaagaaaggtgcacatgatcaccgagaacatcaacgctagctggcgtagcatccacaagagagtgagcgagagagaggcggctttggaggaaacccatagactgctgcagcagtttcccctggacctggagaagttcctggcctggctgaccgaagccgaaacaaccgccaacgtgctgcaagacgccacaagaaaggagagactgctggaggacagcaagggcgtgaaggagctgatgaagcagtggcaagacctgcaaggcgagatcgaagcgcacaccgacgtgtaccacaacctggacgagaactctcagaagatcctgagaagcctggagggcagcgacgacgccgtgctgttacagagaaggctggacaacatgaacttcaagtggagcgagctgagaaagaagagcctgaacatcagaagccacctggaggctagcagcgatcagtggaagagactgcacctgagcctgcaagagctgctggtctggcttcagttgaaggacgatgagctgagcagacaagcccccatcggcggcgacttccccgccgtgcagaagcagaacgacgtgcatagagccttcaagagagagctgaagaccaaggagcccgtgatcatgagcaccctggaaacggtgagaatcttcctgaccgagcagcccctggagggcctggagaagctgtaccaagagcctagagagctgccccccgaggagagagctcagaacgtgacaagactgctgagaaagcaagccgaagaggtgaacaccgagtgggagaagctgaacctgcacagcgccgactggcagagaaagatcgacgaaaccctggagagactgagagaattgcaagaggcgaccgacgagctggacctgaagctgagacaagccgaggtcatcaagggcagctggcagcccgtgggcgatctattaattgactctctacaagaccacctggagaaagtgaaggccctgagaggcgagatcgcccccctgaaggagaacgtgagccacgtgaatgatctggcacgtcagctgaccaccctgggcatacaactgtccccctataacctgagcacactggaggatttgaatactagatggaagctgctgcaagtggccgtggaggacagagtaagacaattacatgaggcccacagagacttcggccccgcttctcagcacttcctgagcacaagcgtgcaaggcccctgggagagagccatttcccccaacaaggtcccctactacatcaaccacgagacacagaccacctgctgggaccaccccaagatgaccgagctgtatcagagcctcgccgatctgaacaatgtgagattcagcgcctacagaaccgccatgaagctgagaagactgcagaaggccctgtgcctggacttattaagcctgagcgccgcctgcgacgccctggatcagcacaacctgaagcaaaacgaccaacccatggacatcctgcagatcatcaactgcctcaccaccatatacgacagactggaacaagagcacaacaacctggtgaacgtgcccctgtgcgtggacatgtgcctgaactggctgctgaacgtgtacgacaccggcagaaccggcagaatcagagtgctgagcttcaagaccggcatcatcagcctgtgcaaggcccacctggaggacaaatacagataccttttcaaacaagtggctagcagcaccggcttctgcgaccaaagaagactgggtctgctgctgcacgacagcattcagatccctagacagctgggcgaggtggctagcttcggcggcagcaacatcgagcctagcgtgagaagctgctttcagttcgccaacaacaagcccgagatagaggcagccctcttcttagactggatgagactggagcctcagagcatggtatggctgccggtcctgcacagagtggccgccgccgaaaccgccaagcaccaagccaagtgcaacatctgcaaggagtgccccatcatcggcttcagatacagaagcctgaagcacttcaactacgacatctgtcagagctgcttcttcagcggcagagtggccaagggccacaagatgcactaccccatggtggagtactgcacccccaccacaagcggcgaggacgtgagagacttcgccaaggtgttgaagaataagtttagaaccaagagatacttcgccaagcaccctagaatgggctacctgcccgtgcagaccgtgctggagggcgacaacatggaaacccccgtgaccctgatcaacttctggcccgtggacagcgcccccgctagcagccctcagctgagccacgacgacacccacagcagaatcgagcactacgctagcagactggccgagatggagaacagcaacggcagctacctgaacgacagcatcagcccgaatgagagcatcgacgacgagcacctgctgattcagcactactgtcagagcctgaaccaagacagccccctgtctcagcctagaagccccgctcagatcctgatcagcctggagagcgaggagagaggcgagctggagagaatcctagcagatctggaggaagagaacagaaacctgcaagccgagtatgaccgactgaaacagcaacatgagcacaaaggcctgagcccactgccctccccccccgagatgatgcccacaagccctcagagccctagagacgccgagctgatcgctgaagcgaagttacttagacaacacaagggcagactggaggctagaatgcagatcctggaggaccacaacaagcagctggagtctcagctgcacagactgaggcagctgctggaacagccccaagccgaggccaaagtgaacggcaccaccgtgagcagtcctagcacaagcctgcagagaagcgacagctctcagcccatgctgctgagagtggtgggctctcagacaagcgacagcatgggcgaggaggatctgctgagtccgccccaagacacaagcaccggcctggaagaggtgatggagcaactgaacaatagcttccctagcagcagaggcagaaacacccccggcaagcccatgagagaggacaccatgtgatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 7 - DNA, 12889 bp, synthetic construct
  source 1..12889: mol_type = other DNA; note = MHCK7-hBGi-DMD-4-WPRE3-DTS
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 8 - DNA, 382 bp, synthetic construct
  source 1..382: mol_type = other DNA; note = WPRE3
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgg

SEQ ID NO: 9 - DNA, 224 bp, synthetic construct
  source 1..224: mol_type = other DNA; note = PolyA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - DNA, 608 bp, synthetic construct
  source 1..608: mol_type = other DNA; note = Post-Tx Reg.
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 11 - DNA, 150 bp, synthetic construct
  source 1..150: mol_type = other DNA; note = DTS
gagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 12 - DNA, 771 bp, synthetic construct
  source 1..771: mol_type = other DNA; note = Post-Tx Reg. DTS
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 13 - protein, 3685 aa, synthetic construct
  source 1..3685: note = Dystrophin -WT
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 14 - DNA, 13222 bp, synthetic construct
  source 1..13222: mol_type = other DNA; note = NP-CBA-hBGi-DMD-3-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggactgttacgagagagaggacgtgcagaagaagacattcaccaagtgggtgaacgctcagttcagcaagttcggcaagcagcacatcgagaacctgtttagcgatctacaagatggcagaagactgctggacctgctggagggcctgaccgggcagaagctgcccaaagaaaaggggtctacaagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaagtgaagaacgtgatgaagaacatcatggccggcctgcagcagaccaacagcgagaagatcctgctgagctgggtgagacagagcacaagaaactacccccaagtgaacgtgatcaacttcaccacaagctggagcgacggcctggccctgaacgccctgatccacagccacagacccgacctgttcgactggaacagcgtggtgtgtcagcagagcgccacacagagactggagcacgccttcaacatcgctagatatcagctgggcatcgagaagttactggaccccgaagatgtggacaccacctaccccgacaagaagagcatcctgatgtacatcacaagcctgttccaagtgctgcctcagcaagtgagcatcgaggccatccaagaagtcgagatgctgcctagaccccccaaggtgaccaaggaggagcactttcagctgcaccatcagatgcactactctcagcagatcacagtcagcttggcacaaggctacgagagaacaagcagccccaagcctagattcaagagctacgcctacacccaagccgcctacgtgaccacaagtgaccccacaagaagccccttcccttctcagcacctggaggcccccgaggacaagagcttcggcagcagcctgatggagagcgaggtgaacctggacagatatcagaccgcgttagaagaagtgttgagctggctgctgagcgccgaggacaccctgcaagcccaaggcgagatcagcaacgatgtggaggtggtgaaggatcagttccacacccacgagggctacatgatggacctgaccgcccaccaaggcagagtgggcaacatcctgcagctgggcagcaagctgatcggcaccggcaagctgagcgaggacgaggaaaccgaggttcaagagcaaatgaacctgctaaacagcagatgggagtgcctgagagtggctagcatggaaaagcagagtaacctgcacagagtgctgatggacctgcagaatcagaaattgaaagaacttaatgattggctgaccaagaccgaggagagaacaagaaagatggaggaggagcccctgggccccgacctggaggacctgaagagacaagtgcagcagcacaaggtgctgcaagaggacctggagcaagagcaagtgagagtgaactctctgacacacatggtggtggtagtggacgagagcagcggcgatcacgctaccgcggcgctggaagagcagctgaaagtgctgggcgacagatgggccaacatctgcagatggaccgaggacagatgggtgctgctgcaagacatcctgctgaagtggcagagactgaccgaggagcagtgcctgttcagcgcctggctgagcgagaaggaagacgctgtgaacaagatccacaccaccggcttcaaggatcagaacgagatgctgagctccctccaaaagctcgcagtgctgaaggccgacctggaaaagaaaaaacagtcgatgggcaagctgtacagcctgaagcaagacctgctgagcaccctgaagaacaagagcgtgacacagaagaccgaggcctggctggacaacttcgctagatgctgggacaacctggtacagaagttagagaagtctaccgctcagatcagccaagccgtaaccaccacacaacctagcctgactcagaccaccgtgatggaaaccgtgaccaccgtgacaacccgtgagcagatcctggtgaagcacgcccaagaggagctgcccccccccccccctcagaagaagagacagatcaccgtggactcagagattcgcaagagactggacgtggacatcaccgagctgcacagctggatcacaagaagcgaggccgtgctgcagagccctgagttcgcgatctttagaaaggagggcaacttcagcgacttgaaggagaaagtgaacgccatcgagagagagaaggccgagaagttcagaaagttgcaagacgcctcaagaagcgcccaagccctggtggagcagatggttaacgaaggcgtgaatgccgacagcatcaagcaagctagcgagcagctgaatagccggtggattgaattctgtcagctgctgagcgagagattaaactggctggagtatcagaacaacatcatcgccttctacaatcagctgcagcaactagaacaaatgaccaccaccgccgagaactggctaaagattcagcccaccacccctagcgagcccaccgccatcaaatctcagctgaaaatttgcaaggacgaagtgaacagactgtcggacctgcagcctcaaatcgaaagactgaaaattcagagcatcgcacttaaggagaagggccaaggacccatgttcctggacgccgacttcgtggccttcaccaaccacttcaagcaagtgttcagcgacgtgcaagctagagagaaggagctgcagaccatcttcgacaccctgccccccatgagataccaagaaaccatgagcgccatcagaacctgggtgcagcagagcgaaaccaagctgagcatccctcagctgagcgtgaccgactacgagatcatggagcaaagactgggagagctgcaagccctgcagagcagcctgcaagagcagcagagcggcctgtactacctgagcaccaccgtgaaggagatgtcgaagaaagccccgtcggagatcagcagaaagtatcagagcgagttcgaggagatcgagggcagatggaagaagctgagcagccaactggtggaacactgtcagaaattagaagaacagatgaacaagctgagaaagattcagaaccacattcagaccctgaagaagtggatggccgaggtggacgtgttcctgaaagaggagtggcccgccctgggcgattccgaaatcttaaaaaagcagctgaagcagtgcagactgctggtgtcagatatccaaaccattcaaccaagcctgaacagcgtgaacgaaggcgggcagaagatcaagaacgaggccgagcccgagttcgctagcagactggaaaccgagctgaaggagctgaatacacagtgggaccacatgtgtcagcaagtgtacgctagaaaggaagcgctgaagggcgggctggagaagaccgttagcctgcaaaaggatctgagcgagatgcacgagtggatgacccaagccgaggaggagtacctggagagagacttcgagtacaagacccccgacgagctgcagaaggccgtcgaggaaatgaagagagcgaaggaggaggctcagcagaaggaagccaaggtgaagctgctgactgaatctgtcaacagtgtgatagctcaagcaccccccgttgcccaagaggcactaaagaaggagctggaaactctgaccaccaactatcagtggctgtgcacaagactgaacggcaagtgcaagaccctggaggaagtgtgggcctgctggcacgagctgctgagctacctggagaaggccaacaagtggctgaacgaggtggagttcaagctgaagaccaccgagaacatccccggcggcgccgaggagatcagcgaggtgctggacagcctggagaacctgatgagacacagcgaggacaaccccaatcagatcagaatcctggctcagaccctgaccgacggcggcgtgatggacgagctgatcaacgaggagctggaaacctttaactctagatggcgagaactgcacgaggaggccgtgagaagacagaagctgctggagcaaagcattcagagcgcccaagaaaccgagaagagcctgcacctgatccaagagagcctgaccttcatcgacaagcagctggccgcctacatcgccgacaaggtggacgccgctcagatgccacaagaggctcagaagattcagtccgatctgacaagccacgagatcagcctggaggagatgaaaaaacacaaccaaggcaaggaggccgctcagagagtgctgtctcagatcgacgtggctcagaagaagctgcaagacgtgagcatgaagttcagactgtttcagaagcccgccaacttcgagcagcgactgcaagagagcaagatgatcctggacgaggtgaagatgcacctgcccgccctggaaaccaagagcgtggagcaagaggtggtgcagtctcagttgaaccactgcgtgaacctgtacaagagcctgtcggaggtcaagagtgaggtcgagatggtgatcaaaactgggagacagatcgtgcaaaaaaagcagaccgagaaccccaaggagctggacgagcgtgttaccgccctgaagctgcactacaacgagctgggcgccaaggtgaccgagagaaagcagcagctggagaagtgcctgaagctgagcagaaagatgagaaaggagatgaacgtgctgaccgagtggctggccgccaccgacatggagctgaccaagagaagcgccgtggagggcatgcctagcaacctggacagcgaggtggcctggggcaaggccacacagaaggagatcgagaagcagaaggtgcacctgaagagcatcaccgaggtgggcgaggccctgaagaccgtgctgggcaagaaggaaaccctggtggaggacaagctgagcctgctgaactcgaactggatcgccgtgacaagcagagccgaggagtggctgaacctgctgttagagtaccaaaaacacatggaaaccttcgatcagaacgtggaccacatcaccaagtggatcatccaagccgacaccctgctggacgaaagcgaaaagaagaagccgcaacaaaaggaggatgtgctgaagagactgaaggccgaactgaacgatatcagacccaaggtggacagcacacgggaccaagccgccaacctgatggccaacagaggcgaccactgcagaaaactggtggagcctcagatcagcgagctgaaccacagattcgccgccatcagccacagaatcaaaaccggcaaagcgagcatccccttgaaggaactggagcaatttaacagcgacatccaaaaattgctcgaaccactggaggccgagattcagcaaggcgtgaacctgaaggaagaggacttcaacaaggacatgaacgaggacaacgagggcaccgtgaaagaactgctgcagagaggcgacaacctgcagcagagaatcaccgacgagagaaagagagaggagatcaagatcaagcagcagctgctgcagactaagcacaacgccctgaaggatctgagatctcagaggagaaaaaaggctctggaaatttcacatcagtggtatcagtacaagagacaagcagacgacctgctgaagtgcctggacgacatcgagaagaagttagctagcctgcccgagcctagagacgagagaaagatcaaggagatcgacagagagttacaaaagaagaaggaggagctgaatgcggtaagaagacaagcggaaggcctgtccgaggacggcgcagccatggccgtggagcccacacagattcagctcagcaagagatggagagagatcgagagcaagttcgctcagttcagaagactgaacttcgctcagatccacaccgtgagagaggaaaccatgatggtgatgaccgaggacatgcccctggagatctcatacgttcctagcacctacctgaccgagatcacccacgtgagccaagccctgctggaggtggagcagctgctgaacgcccccgacctgtgcgccaaggacttcgaggacctgttcaaacaagaggagagcctgaagaacatcaaggacagcctgcagcaaagtagcggcagaatcgacatcatccacagcaagaagaccgccgccctgcagagcgccacccccgtggagagagtgaagcttcaagaggccctttcgcagctggattttcagtgggagaaggtgaacaaaatgtacaaggacagacaaggcagattcgacagaagcgtggagaagtggagaagattccactacgacatcaagatcttcaatcagtggctgaccgaggcagagcagttcctgagaaagacacagatccccgagaactgggagcacgccaagtacaagtggtacctgaaggagctgcaagacggcatcgggcagagacagaccgtggtgagaaccctgaacgccaccggcgaggagatcatccaacaaagctccaaaaccgacgctagcatcctgcaagagaaactgggcagcctgaacctgagatggcaagaggtgtgcaagcagctgagcgaccgcaaaaaacggttagaggagcagaagaacatcctgagcgagtttcagcgggacctgaacgagttcgtgctgtggctggaggaggccgataacatcgctagcatccccctggagcccggcaaggagcagcagttaaaagaaaaacttgagcaagtgaagctgctggtggaggagctgcccctgagacaaggcatcctgaagcagctgaacgagactggcggccccgtgctggtgagcgcccccatcagccccgaggagcaagacaagctggagaacaagctgaagcagaccaacctgcagtggatcaaggtgagcagagccctgcccgagaagcaaggcgaaatcgaggcccaaataaaggacctggggcagctggagaagaaactcgaagacttggaagaacaactcaaccacttgctgctgtggctgagccccatcagaaatcagctggagatctacaatcagcccaaccaagaggggccattcgacgtgaaggaaaccgagatcgccgtgcaagccaagcagcccgatgtggaggagatcctgagcaaggggcagcacctgtacaaggagaagcccgccacacagcccgtcaagcgtaaactagaggatctttcctctgagtggaaggccgtgaaccgtctgctacaagagctgcgggctaagcaacccgacttagcgcccggcctgaccaccatcggtgcaagccccacgcagaccgtgaccctggtgacccaacccgtggtgaccaaggaaaccgccatcagcaagctggagatgcctagcagcctgatgctggaggtgcccgccctggccgacttcaacagagcctggaccgagctgaccgactggctgagcctgctggaccaagtgatcaagtctcagagagtgatggtgggcgatctggaggacatcaatgagatgatcatcaagcagaaggccaccatgcaagacctggagcagagaagacctcagctggaggagctgatcaccgccgctcagaacctgaaaaacaagacgagcaaccaagaggctagaaccatcatcaccgacagaatcgagagaattcagaatcagtgggacgaggtgcaagagcacctgcagaacagaagacagcaactaaacgaaatgctcaaggacagcacacagtggctggaggccaaggaggaggcggagcaagtgttgggccaagctagagccaagctggagagctggaaggagggaccctacaccgtggacgccattcagaagaagatcaccgaaaccaagcagctggccaaggacctgcgacagtggcagaccaacgtggacgtggccaacgatcttgccctgaagctgctgcgcgactatagcgccgacgacacaagaaaggtgcacatgatcaccgagaacatcaacgctagctggcgtagcatccacaagagagtgagcgagagagaggcggctttggaggaaacccatagactgctgcagcagtttcccctggacctggagaagttcctggcctggctgaccgaagccgaaacaaccgccaacgtgctgcaagacgccacaagaaaggagagactgctggaggacagcaagggcgtgaaggagctgatgaagcagtggcaagacctgcaaggcgagatcgaagcgcacaccgacgtgtaccacaacctggacgagaactctcagaagatcctgagaagcctggagggcagcgacgacgccgtgctgttacagagaaggctggacaacatgaacttcaagtggagcgagctgagaaagaagagcctgaacatcagaagccacctggaggctagcagcgatcagtggaagagactgcacctgagcctgcaagagctgctggtctggcttcagttgaaggacgatgagctgagcagacaagcccccatcggcggcgacttccccgccgtgcagaagcagaacgacgtgcatagagccttcaagagagagctgaagaccaaggagcccgtgatcatgagcaccctggaaacggtgagaatcttcctgaccgagcagcccctggagggcctggagaagctgtaccaagagcctagagagctgccccccgaggagagagctcagaacgtgacaagactgctgagaaagcaagccgaagaggtgaacaccgagtgggagaagctgaacctgcacagcgccgactggcagagaaagatcgacgaaaccctggagagactgagagaattgcaagaggcgaccgacgagctggacctgaagctgagacaagccgaggtcatcaagggcagctggcagcccgtgggcgatctattaattgactctctacaagaccacctggagaaagtgaaggccctgagaggcgagatcgcccccctgaaggagaacgtgagccacgtgaatgatctggcacgtcagctgaccaccctgggcatacaactgtccccctataacctgagcacactggaggatttgaatactagatggaagctgctgcaagtggccgtggaggacagagtaagacaattacatgaggcccacagagacttcggccccgcttctcagcacttcctgagcacaagcgtgcaaggcccctgggagagagccatttcccccaacaaggtcccctactacatcaaccacgagacacagaccacctgctgggaccaccccaagatgaccgagctgtatcagagcctcgccgatctgaacaatgtgagattcagcgcctacagaaccgccatgaagctgagaagactgcagaaggccctgtgcctggacttattaagcctgagcgccgcctgcgacgccctggatcagcacaacctgaagcaaaacgaccaacccatggacatcctgcagatcatcaactgcctcaccaccatatacgacagactggaacaagagcacaacaacctggtgaacgtgcccctgtgcgtggacatgtgcctgaactggctgctgaacgtgtacgacaccggcagaaccggcagaatcagagtgctgagcttcaagaccggcatcatcagcctgtgcaaggcccacctggaggacaaatacagataccttttcaaacaagtggctagcagcaccggcttctgcgaccaaagaagactgggtctgctgctgcacgacagcattcagatccctagacagctgggcgaggtggctagcttcggcggcagcaacatcgagcctagcgtgagaagctgctttcagttcgccaacaacaagcccgagatagaggcagccctcttcttagactggatgagactggagcctcagagcatggtatggctgccggtcctgcacagagtggccgccgccgaaaccgccaagcaccaagccaagtgcaacatctgcaaggagtgccccatcatcggcttcagatacagaagcctgaagcacttcaactacgacatctgtcagagctgcttcttcagcggcagagtggccaagggccacaagatgcactaccccatggtggagtactgcacccccaccacaagcggcgaggacgtgagagacttcgccaaggtgttgaagaataagtttagaaccaagagatacttcgccaagcaccctagaatgggctacctgcccgtgcagaccgtgctggagggcgacaacatggaaacccccgtgaccctgatcaacttctggcccgtggacagcgcccccgctagcagccctcagctgagccacgacgacacccacagcagaatcgagcactacgctagcagactggccgagatggagaacagcaacggcagctacctgaacgacagcatcagcccgaatgagagcatcgacgacgagcacctgctgattcagcactactgtcagagcctgaaccaagacagccccctgtctcagcctagaagccccgctcagatcctgatcagcctggagagcgaggagagaggcgagctggagagaatcctagcagatctggaggaagagaacagaaacctgcaagccgagtatgaccgactgaaacagcaacatgagcacaaaggcctgagcccactgccctccccccccgagatgatgcccacaagccctcagagccctagagacgccgagctgatcgctgaagcgaagttacttagacaacacaagggcagactggaggctagaatgcagatcctggaggaccacaacaagcagctggagtctcagctgcacagactgaggcagctgctggaacagccccaagccgaggccaaagtgaacggcaccaccgtgagcagtcctagcacaagcctgcagagaagcgacagctctcagcccatgctgctgagagtggtgggctctcagacaagcgacagcatgggcgaggaggatctgctgagtccgccccaagacacaagcaccggcctggaagaggtgatggagcaactgaacaatagcttccctagcagcagaggcagaaacacccccggcaagcccatgagagaggacaccatgtgatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggcccgtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattatgcggccgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtc

SEQ ID NO: 15 - DNA, 13210 bp, synthetic construct
  source 1..13210: mol_type = other DNA; note = NP-CBA-hBGi-DMD-4-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattacgaatgcatgcggccgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtc

SEQ ID NO: 16 - DNA, 13397 bp, synthetic construct
  source 1..13397: mol_type = other DNA; note = NP-MHCK7-hBGi-DMD-3-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggactgttacgagagagaggacgtgcagaagaagacattcaccaagtgggtgaacgctcagttcagcaagttcggcaagcagcacatcgagaacctgtttagcgatctacaagatggcagaagactgctggacctgctggagggcctgaccgggcagaagctgcccaaagaaaaggggtctacaagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaagtgaagaacgtgatgaagaacatcatggccggcctgcagcagaccaacagcgagaagatcctgctgagctgggtgagacagagcacaagaaactacccccaagtgaacgtgatcaacttcaccacaagctggagcgacggcctggccctgaacgccctgatccacagccacagacccgacctgttcgactggaacagcgtggtgtgtcagcagagcgccacacagagactggagcacgccttcaacatcgctagatatcagctgggcatcgagaagttactggaccccgaagatgtggacaccacctaccccgacaagaagagcatcctgatgtacatcacaagcctgttccaagtgctgcctcagcaagtgagcatcgaggccatccaagaagtcgagatgctgcctagaccccccaaggtgaccaaggaggagcactttcagctgcaccatcagatgcactactctcagcagatcacagtcagcttggcacaaggctacgagagaacaagcagccccaagcctagattcaagagctacgcctacacccaagccgcctacgtgaccacaagtgaccccacaagaagccccttcccttctcagcacctggaggcccccgaggacaagagcttcggcagcagcctgatggagagcgaggtgaacctggacagatatcagaccgcgttagaagaagtgttgagctggctgctgagcgccgaggacaccctgcaagcccaaggcgagatcagcaacgatgtggaggtggtgaaggatcagttccacacccacgagggctacatgatggacctgaccgcccaccaaggcagagtgggcaacatcctgcagctgggcagcaagctgatcggcaccggcaagctgagcgaggacgaggaaaccgaggttcaagagcaaatgaacctgctaaacagcagatgggagtgcctgagagtggctagcatggaaaagcagagtaacctgcacagagtgctgatggacctgcagaatcagaaattgaaagaacttaatgattggctgaccaagaccgaggagagaacaagaaagatggaggaggagcccctgggccccgacctggaggacctgaagagacaagtgcagcagcacaaggtgctgcaagaggacctggagcaagagcaagtgagagtgaactctctgacacacatggtggtggtagtggacgagagcagcggcgatcacgctaccgcggcgctggaagagcagctgaaagtgctgggcgacagatgggccaacatctgcagatggaccgaggacagatgggtgctgctgcaagacatcctgctgaagtggcagagactgaccgaggagcagtgcctgttcagcgcctggctgagcgagaaggaagacgctgtgaacaagatccacaccaccggcttcaaggatcagaacgagatgctgagctccctccaaaagctcgcagtgctgaaggccgacctggaaaagaaaaaacagtcgatgggcaagctgtacagcctgaagcaagacctgctgagcaccctgaagaacaagagcgtgacacagaagaccgaggcctggctggacaacttcgctagatgctgggacaacctggtacagaagttagagaagtctaccgctcagatcagccaagccgtaaccaccacacaacctagcctgactcagaccaccgtgatggaaaccgtgaccaccgtgacaacccgtgagcagatcctggtgaagcacgcccaagaggagctgcccccccccccccctcagaagaagagacagatcaccgtggactcagagattcgcaagagactggacgtggacatcaccgagctgcacagctggatcacaagaagcgaggccgtgctgcagagccctgagttcgcgatctttagaaaggagggcaacttcagcgacttgaaggagaaagtgaacgccatcgagagagagaaggccgagaagttcagaaagttgcaagacgcctcaagaagcgcccaagccctggtggagcagatggttaacgaaggcgtgaatgccgacagcatcaagcaagctagcgagcagctgaatagccggtggattgaattctgtcagctgctgagcgagagattaaactggctggagtatcagaacaacatcatcgccttctacaatcagctgcagcaactagaacaaatgaccaccaccgccgagaactggctaaagattcagcccaccacccctagcgagcccaccgccatcaaatctcagctgaaaatttgcaaggacgaagtgaacagactgtcggacctgcagcctcaaatcgaaagactgaaaattcagagcatcgcacttaaggagaagggccaaggacccatgttcctggacgccgacttcgtggccttcaccaaccacttcaagcaagtgttcagcgacgtgcaagctagagagaaggagctgcagaccatcttcgacaccctgccccccatgagataccaagaaaccatgagcgccatcagaacctgggtgcagcagagcgaaaccaagctgagcatccctcagctgagcgtgaccgactacgagatcatggagcaaagactgggagagctgcaagccctgcagagcagcctgcaagagcagcagagcggcctgtactacctgagcaccaccgtgaaggagatgtcgaagaaagccccgtcggagatcagcagaaagtatcagagcgagttcgaggagatcgagggcagatggaagaagctgagcagccaactggtggaacactgtcagaaattagaagaacagatgaacaagctgagaaagattcagaaccacattcagaccctgaagaagtggatggccgaggtggacgtgttcctgaaagaggagtggcccgccctgggcgattccgaaatcttaaaaaagcagctgaagcagtgcagactgctggtgtcagatatccaaaccattcaaccaagcctgaacagcgtgaacgaaggcgggcagaagatcaagaacgaggccgagcccgagttcgctagcagactggaaaccgagctgaaggagctgaatacacagtgggaccacatgtgtcagcaagtgtacgctagaaaggaagcgctgaagggcgggctggagaagaccgttagcctgcaaaaggatctgagcgagatgcacgagtggatgacccaagccgaggaggagtacctggagagagacttcgagtacaagacccccgacgagctgcagaaggccgtcgaggaaatgaagagagcgaaggaggaggctcagcagaaggaagccaaggtgaagctgctgactgaatctgtcaacagtgtgatagctcaagcaccccccgttgcccaagaggcactaaagaaggagctggaaactctgaccaccaactatcagtggctgtgcacaagactgaacggcaagtgcaagaccctggaggaagtgtgggcctgctggcacgagctgctgagctacctggagaaggccaacaagtggctgaacgaggtggagttcaagctgaagaccaccgagaacatccccggcggcgccgaggagatcagcgaggtgctggacagcctggagaacctgatgagacacagcgaggacaaccccaatcagatcagaatcctggctcagaccctgaccgacggcggcgtgatggacgagctgatcaacgaggagctggaaacctttaactctagatggcgagaactgcacgaggaggccgtgagaagacagaagctgctggagcaaagcattcagagcgcccaagaaaccgagaagagcctgcacctgatccaagagagcctgaccttcatcgacaagcagctggccgcctacatcgccgacaaggtggacgccgctcagatgccacaagaggctcagaagattcagtccgatctgacaagccacgagatcagcctggaggagatgaaaaaacacaaccaaggcaaggaggccgctcagagagtgctgtctcagatcgacgtggctcagaagaagctgcaagacgtgagcatgaagttcagactgtttcagaagcccgccaacttcgagcagcgactgcaagagagcaagatgatcctggacgaggtgaagatgcacctgcccgccctggaaaccaagagcgtggagcaagaggtggtgcagtctcagttgaaccactgcgtgaacctgtacaagagcctgtcggaggtcaagagtgaggtcgagatggtgatcaaaactgggagacagatcgtgcaaaaaaagcagaccgagaaccccaaggagctggacgagcgtgttaccgccctgaagctgcactacaacgagctgggcgccaaggtgaccgagagaaagcagcagctggagaagtgcctgaagctgagcagaaagatgagaaaggagatgaacgtgctgaccgagtggctggccgccaccgacatggagctgaccaagagaagcgccgtggagggcatgcctagcaacctggacagcgaggtggcctggggcaaggccacacagaaggagatcgagaagcagaaggtgcacctgaagagcatcaccgaggtgggcgaggccctgaagaccgtgctgggcaagaaggaaaccctggtggaggacaagctgagcctgctgaactcgaactggatcgccgtgacaagcagagccgaggagtggctgaacctgctgttagagtaccaaaaacacatggaaaccttcgatcagaacgtggaccacatcaccaagtggatcatccaagccgacaccctgctggacgaaagcgaaaagaagaagccgcaacaaaaggaggatgtgctgaagagactgaaggccgaactgaacgatatcagacccaaggtggacagcacacgggaccaagccgccaacctgatggccaacagaggcgaccactgcagaaaactggtggagcctcagatcagcgagctgaaccacagattcgccgccatcagccacagaatcaaaaccggcaaagcgagcatccccttgaaggaactggagcaatttaacagcgacatccaaaaattgctcgaaccactggaggccgagattcagcaaggcgtgaacctgaaggaagaggacttcaacaaggacatgaacgaggacaacgagggcaccgtgaaagaactgctgcagagaggcgacaacctgcagcagagaatcaccgacgagagaaagagagaggagatcaagatcaagcagcagctgctgcagactaagcacaacgccctgaaggatctgagatctcagaggagaaaaaaggctctggaaatttcacatcagtggtatcagtacaagagacaagcagacgacctgctgaagtgcctggacgacatcgagaagaagttagctagcctgcccgagcctagagacgagagaaagatcaaggagatcgacagagagttacaaaagaagaaggaggagctgaatgcggtaagaagacaagcggaaggcctgtccgaggacggcgcagccatggccgtggagcccacacagattcagctcagcaagagatggagagagatcgagagcaagttcgctcagttcagaagactgaacttcgctcagatccacaccgtgagagaggaaaccatgatggtgatgaccgaggacatgcccctggagatctcatacgttcctagcacctacctgaccgagatcacccacgtgagccaagccctgctggaggtggagcagctgctgaacgcccccgacctgtgcgccaaggacttcgaggacctgttcaaacaagaggagagcctgaagaacatcaaggacagcctgcagcaaagtagcggcagaatcgacatcatccacagcaagaagaccgccgccctgcagagcgccacccccgtggagagagtgaagcttcaagaggccctttcgcagctggattttcagtgggagaaggtgaacaaaatgtacaaggacagacaaggcagattcgacagaagcgtggagaagtggagaagattccactacgacatcaagatcttcaatcagtggctgaccgaggcagagcagttcctgagaaagacacagatccccgagaactgggagcacgccaagtacaagtggtacctgaaggagctgcaagacggcatcgggcagagacagaccgtggtgagaaccctgaacgccaccggcgaggagatcatccaacaaagctccaaaaccgacgctagcatcctgcaagagaaactgggcagcctgaacctgagatggcaagaggtgtgcaagcagctgagcgaccgcaaaaaacggttagaggagcagaagaacatcctgagcgagtttcagcgggacctgaacgagttcgtgctgtggctggaggaggccgataacatcgctagcatccccctggagcccggcaaggagcagcagttaaaagaaaaacttgagcaagtgaagctgctggtggaggagctgcccctgagacaaggcatcctgaagcagctgaacgagactggcggccccgtgctggtgagcgcccccatcagccccgaggagcaagacaagctggagaacaagctgaagcagaccaacctgcagtggatcaaggtgagcagagccctgcccgagaagcaaggcgaaatcgaggcccaaataaaggacctggggcagctggagaagaaactcgaagacttggaagaacaactcaaccacttgctgctgtggctgagccccatcagaaatcagctggagatctacaatcagcccaaccaagaggggccattcgacgtgaaggaaaccgagatcgccgtgcaagccaagcagcccgatgtggaggagatcctgagcaaggggcagcacctgtacaaggagaagcccgccacacagcccgtcaagcgtaaactagaggatctttcctctgagtggaaggccgtgaaccgtctgctacaagagctgcgggctaagcaacccgacttagcgcccggcctgaccaccatcggtgcaagccccacgcagaccgtgaccctggtgacccaacccgtggtgaccaaggaaaccgccatcagcaagctggagatgcctagcagcctgatgctggaggtgcccgccctggccgacttcaacagagcctggaccgagctgaccgactggctgagcctgctggaccaagtgatcaagtctcagagagtgatggtgggcgatctggaggacatcaatgagatgatcatcaagcagaaggccaccatgcaagacctggagcagagaagacctcagctggaggagctgatcaccgccgctcagaacctgaaaaacaagacgagcaaccaagaggctagaaccatcatcaccgacagaatcgagagaattcagaatcagtgggacgaggtgcaagagcacctgcagaacagaagacagcaactaaacgaaatgctcaaggacagcacacagtggctggaggccaaggaggaggcggagcaagtgttgggccaagctagagccaagctggagagctggaaggagggaccctacaccgtggacgccattcagaagaagatcaccgaaaccaagcagctggccaaggacctgcgacagtggcagaccaacgtggacgtggccaacgatcttgccctgaagctgctgcgcgactatagcgccgacgacacaagaaaggtgcacatgatcaccgagaacatcaacgctagctggcgtagcatccacaagagagtgagcgagagagaggcggctttggaggaaacccatagactgctgcagcagtttcccctggacctggagaagttcctggcctggctgaccgaagccgaaacaaccgccaacgtgctgcaagacgccacaagaaaggagagactgctggaggacagcaagggcgtgaaggagctgatgaagcagtggcaagacctgcaaggcgagatcgaagcgcacaccgacgtgtaccacaacctggacgagaactctcagaagatcctgagaagcctggagggcagcgacgacgccgtgctgttacagagaaggctggacaacatgaacttcaagtggagcgagctgagaaagaagagcctgaacatcagaagccacctggaggctagcagcgatcagtggaagagactgcacctgagcctgcaagagctgctggtctggcttcagttgaaggacgatgagctgagcagacaagcccccatcggcggcgacttccccgccgtgcagaagcagaacgacgtgcatagagccttcaagagagagctgaagaccaaggagcccgtgatcatgagcaccctggaaacggtgagaatcttcctgaccgagcagcccctggagggcctggagaagctgtaccaagagcctagagagctgccccccgaggagagagctcagaacgtgacaagactgctgagaaagcaagccgaagaggtgaacaccgagtgggagaagctgaacctgcacagcgccgactggcagagaaagatcgacgaaaccctggagagactgagagaattgcaagaggcgaccgacgagctggacctgaagctgagacaagccgaggtcatcaagggcagctggcagcccgtgggcgatctattaattgactctctacaagaccacctggagaaagtgaaggccctgagaggcgagatcgcccccctgaaggagaacgtgagccacgtgaatgatctggcacgtcagctgaccaccctgggcatacaactgtccccctataacctgagcacactggaggatttgaatactagatggaagctgctgcaagtggccgtggaggacagagtaagacaattacatgaggcccacagagacttcggccccgcttctcagcacttcctgagcacaagcgtgcaaggcccctgggagagagccatttcccccaacaaggtcccctactacatcaaccacgagacacagaccacctgctgggaccaccccaagatgaccgagctgtatcagagcctcgccgatctgaacaatgtgagattcagcgcctacagaaccgccatgaagctgagaagactgcagaaggccctgtgcctggacttattaagcctgagcgccgcctgcgacgccctggatcagcacaacctgaagcaaaacgaccaacccatggacatcctgcagatcatcaactgcctcaccaccatatacgacagactggaacaagagcacaacaacctggtgaacgtgcccctgtgcgtggacatgtgcctgaactggctgctgaacgtgtacgacaccggcagaaccggcagaatcagagtgctgagcttcaagaccggcatcatcagcctgtgcaaggcccacctggaggacaaatacagataccttttcaaacaagtggctagcagcaccggcttctgcgaccaaagaagactgggtctgctgctgcacgacagcattcagatccctagacagctgggcgaggtggctagcttcggcggcagcaacatcgagcctagcgtgagaagctgctttcagttcgccaacaacaagcccgagatagaggcagccctcttcttagactggatgagactggagcctcagagcatggtatggctgccggtcctgcacagagtggccgccgccgaaaccgccaagcaccaagccaagtgcaacatctgcaaggagtgccccatcatcggcttcagatacagaagcctgaagcacttcaactacgacatctgtcagagctgcttcttcagcggcagagtggccaagggccacaagatgcactaccccatggtggagtactgcacccccaccacaagcggcgaggacgtgagagacttcgccaaggtgttgaagaataagtttagaaccaagagatacttcgccaagcaccctagaatgggctacctgcccgtgcagaccgtgctggagggcgacaacatggaaacccccgtgaccctgatcaacttctggcccgtggacagcgcccccgctagcagccctcagctgagccacgacgacacccacagcagaatcgagcactacgctagcagactggccgagatggagaacagcaacggcagctacctgaacgacagcatcagcccgaatgagagcatcgacgacgagcacctgctgattcagcactactgtcagagcctgaaccaagacagccccctgtctcagcctagaagccccgctcagatcctgatcagcctggagagcgaggagagaggcgagctggagagaatcctagcagatctggaggaagagaacagaaacctgcaagccgagtatgaccgactgaaacagcaacatgagcacaaaggcctgagcccactgccctccccccccgagatgatgcccacaagccctcagagccctagagacgccgagctgatcgctgaagcgaagttacttagacaacacaagggcagactggaggctagaatgcagatcctggaggaccacaacaagcagctggagtctcagctgcacagactgaggcagctgctggaacagccccaagccgaggccaaagtgaacggcaccaccgtgagcagtcctagcacaagcctgcagagaagcgacagctctcagcccatgctgctgagagtggtgggctctcagacaagcgacagcatgggcgaggaggatctgctgagtccgccccaagacacaagcaccggcctggaagaggtgatggagcaactgaacaatagcttccctagcagcagaggcagaaacacccccggcaagcccatgagagaggacaccatgtgatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggcccgtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattatctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 17 - DNA, 13385 bp, synthetic construct
  source 1..13385: mol_type = other DNA; note = NP-MHCK7-hBGi-DMD-4-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattacgaatgcatctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 18 - DNA, 868 bp, synthetic construct
  source 1..868: mol_type = other DNA; note = CBA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 19 - DNA, 11061 bp, synthetic construct
  source 1..11061: mol_type = other DNA; note = DMD-1
atgctgtggtgggaagaggtggaggattgctacgagcgggaggatgtgcagaaaaagactttcaccaagtgggtaaatgctcagttctctaagttcgggaagcaacacatcgagaacttgttttccgacctccaggatggcaggcgcctgcttgacttgctggagggtctgactggtcagaagctccctaaggagaagggcagcaccagagttcacgctctgaataacgttaacaaagccctgagggtcctccagaataacaatgtagacctggtgaacatcggttctaccgacatcgtggacgggaatcataaactgaccttggggctcatctggaacattatcttgcactggcaggtgaagaacgtcatgaagaatatcatggccgggctgcaacagaccaactccgaaaagattttgctcagctgggtgcgccagtccacccgcaattatccacaagtcaacgttatcaatttcaccactagttggagtgatggcctcgccctgaacgccttgatccacagtcaccgccccgacctgtttgactggaactctgtggtttgccaacagagcgctacccagcgtctcgaacatgctttcaatatcgctagataccagctcggtatcgagaagctgttggatcctgaggacgtagacacgacctaccccgataaaaagagcatcctgatgtatatcacttctctgtttcaggtgcttcctcaacaggtgtccattgaagccatccaagaggtcgaaatgctgcccaggccccctaaggtgacaaaagaagagcatttccagctgcatcaccagatgcattacagtcaacagattaccgtcagcttggctcagggatatgagcgcacgtcctctcctaagcccagatttaagtcctacgcatatacccaggctgcctacgtgaccacttccgacccaactaggagcccctttccctcccaacacctggaggcccctgaggacaaaagcttcggatctagtctgatggagtccgaggtgaatctggatcggtaccaaactgctctggaggaagtgctgtcctggcttctgtccgctgaagacaccctgcaggcgcaaggtgagatttctaacgacgtcgaggtcgtgaaggatcagttccacactcatgagggctacatgatggacttgacagctcaccagggaagagttggtaacatcctgcagcttggctccaagttgattggcacgggaaagcttagcgaggacgaggaaaccgaggtacaggagcagatgaacttgctgaattcccgctgggagtgcctgagagttgcgtccatggaaaagcagtcaaaccttcacagagtgctgatggaccttcagaaccagaagctgaaggagctgaacgattggctgacaaagaccgaggaacgcacaagaaaaatggaagaggaacctctgggacctgatctggaggatctgaagcgccaggtccaacagcataaggtgctgcaggaggatttggagcaggagcaggtgcgggtcaacagcctgacacatatggtggttgtggtcgatgagtcctctggcgatcatgctaccgcagcgctggaagagcagctgaaggtgctgggggatcgctgggccaacatttgcagatggacagaagaccggtgggtcctgcttcaagatatcctgctcaagtggcagcgcctgactgaagagcagtgcctgttctcagcttggttgagcgaaaaagaggacgcagttaacaagatccataccacaggcttcaaggatcagaatgagatgctgagctcactgcagaagctggctgtgctgaaggccgacttggagaagaaaaagcagtccatgggcaaactgtatagcctgaagcaggacctgctcagcaccctgaagaacaaaagcgttacgcagaaaactgaggcctggttggacaactttgctcgctgctgggataaccttgtgcagaagctggaaaagtcaaccgcacagatctcccaggctgtgaccactacgcagcctagcttgacccagacaaccgttatggaaaccgtgacaaccgtgacaacgagagagcaaatcctggttaagcatgcccaggaggaactgccgccacccccgcctcagaagaaacggcagattacggtggacagcgagatccgcaagaggctggacgtcgacatcactgagctccactcatggatcacgcggagtgaggccgtgctgcagtctcccgagttcgctattttccgcaaggagggaaacttctctgacttgaaagagaaggtgaatgccatcgagcgtgaaaaggcagaaaagttcagaaagctgcaggacgcctctcgctctgcgcaggccctggtggagcagatggtcaacgagggtgttaacgctgacagcatcaaacaggcaagcgaacagctgaactccagatggatcgaattttgccaactgctctccgaacgcctcaactggttggagtaccagaacaatattatcgctttttacaaccagctgcagcaactggagcagatgacaactaccgccgaaaactggctgaaaattcagccaaccacaccgtccgagccaactgcgatcaagtctcagctgaagatctgcaaggacgaagtgaaccgtcttagcgacttgcaaccccagattgaacggctcaagattcagtctattgctctgaaggagaagggtcagggtccgatgttcctggatgcagattttgtcgcatttaccaatcacttcaaacaggtgtttagtgacgtccaggcgcgcgagaaagagctgcagactatttttgacaccctgcctcccatgaggtaccaggagacaatgagtgccattcgcacctgggtgcaacagtccgagacgaaattgtcaatcccacagctgtccgtgacagactacgagattatggagcaacggttgggggagctgcaggctcttcagagttccctgcaggagcaacagagcggactgtactatctttccactaccgtgaaggaaatgagtaagaaagccccttccgagatcagccgcaaataccagtctgaattcgaagagatcgagggacgctggaaaaagctgtcaagccagctggtcgagcactgtcagaagctggaagagcagatgaacaagctgcgtaaaatccagaaccatattcagaccctcaagaaatggatggcagaagttgacgtgtttctgaaggaagagtggcctgccctcggcgatagcgaaatccttaagaaacaactcaagcagtgccgcctcctggtgtccgatattcagacaatccagccttccctgaacagcgttaatgagggcggacagaaaattaaaaacgaggccgaacccgagttcgcttctcgcctggagactgaactgaaggagctgaatacccagtgggaccatatgtgtcaacaggtctacgcacgcaaagaggcactgaagggcggtctggagaagactgtctcactccagaaggacctgagcgagatgcacgagtggatgacccaggccgaagaggaatacctggagcgtgattttgagtataagaccccagacgaactccagaaagccgttgaagagatgaagagggctaaggaggaagcccaacagaaggaggcgaaagtgaagctcctgacagagtccgtgaactccgtcatcgctcaagctcctcccgtggctcaggaggccctcaagaaagagctggaaactctgaccacaaattaccagtggttgtgtacccgcttgaacggtaagtgcaagaccctggaagaggtgtgggcctgctggcacgagctcctgtcctatcttgagaaggcgaacaaatggctgaacgaagtggaattcaagttgaaaacaactgagaacattccgggaggcgctgaggaaatctctgaggtgctggactctctggaaaatctgatgcgtcattctgaagacaatcctaaccagatcaggattctggcacagaccctcactgacggaggcgttatggacgaacttatcaacgaggaactggagacattcaattcccgttggcgtgagctgcacgaagaggccgtgcgccgtcagaagttgctggaacagtctatccagtccgcccaggagacagagaaatctctccacctgatccaggagagtctgacgtttatcgataagcagctggctgcctacatcgccgacaaggtggacgctgcccagatgccccaagaggctcagaagatccagtccgacctgacgtctcatgaaatcagcctggaagagatgaaaaagcacaaccagggcaaagaggctgcccagcgcgtcctctcccagattgacgtggctcagaaaaagttgcaggacgtgagcatgaagtttaggctgttccagaaacccgccaacttcgaacagagactgcaggagtctaagatgatcctggatgaggtcaagatgcacctccctgctctggaaacaaaatctgtggagcaggaggtcgtgcagtcacaactgaaccattgtgtgaacctctacaagtctctcagcgaggtgaagtccgaggtcgagatggtgatcaagaccggacgccagattgtgcagaaaaagcagacagagaacccaaaggagctggacgaacgggtgaccgccctgaagctccactacaacgagttgggtgccaaagtcactgaaagaaagcaacagctggagaagtgtctgaagctgagccgcaagatgcgcaaagaaatgaacgtcttgaccgagtggttggcggccaccgacatggagctgaccaagagatccgctgtggagggcatgccctccaaccttgactcagaggtggcctggggcaaggccacacagaaggagatcgaaaaacagaaggtgcatcttaaatcaatcactgaggtgggcgaagctctgaagacagtgctgggcaagaaagagacacttgtagaggacaagctctcccttctgaatagcaattggatcgccgtaactagtcgcgcggaggaatggctgaacctcctgttggaataccagaaacacatggaaaccttcgatcagaacgtggaccatattaccaagtggattatccaggctgacactctcctggacgagtctgagaaaaagaaaccccagcaaaaggaagatgtgctgaagcgcctgaaggcggaattgaacgacatccgcccgaaggttgattccacccgcgaccaagcggccaaccttatggctaaccgcggggaccattgcagaaaactggtggaaccgcagatctctgagctgaaccacagattcgccgcgatttctcacagaattaagactggcaaggcctccatccctctgaaagagctggagcagttcaactctgatattcagaagcttctggagcccctggaggcagaaatccaacagggcgtcaacctgaaagaggaagactttaataaggacatgaatgaggataacgaaggtaccgtcaaggagctcctgcagaggggcgacaatctgcagcaaagaatcactgacgaacgtaagcgcgaagagattaagatcaagcaacagctgttgcagactaagcacaacgccctgaaggatctgaggtcccagcgcaggaaaaaggccctggagatttcccaccagtggtaccagtacaagcgccaggccgatgaccttctgaagtgcctcgacgatatcgagaaaaagctggcctcactgcccgagccccgcgacgagcgcaagatcaaggagattgaccgtgagctccagaagaaaaaggaggaactgaacgcggtgcgcagacaggccgagggcctctccgaggacggcgctgcgatggctgttgagcccactcagatccaactgagcaagcggtggagagagatcgagtctaagttcgcgcaatttaggcgcctcaacttcgcccagattcacacggtccgtgaggaaacgatgatggtgatgacagaggatatgccactggaaatcagctacgtgcccagtacatatctcaccgaaatcacccatgtgtcccaggctctcctggaggtggaacagttgctcaacgctcccgacttgtgcgctaaggacttcgaggacctgttcaagcaggaagagagcctgaaaaacattaaggactccctgcaacagtcaagcggccgcatcgatatcattcattctaagaaaaccgctgccctgcagtctgcaacccccgtagagcgcgtcaaactgcaggaagctctcagccaactggatttccagtgggagaaggtgaacaagatgtataaggatcgtcagggccggttcgaccgctctgtggagaagtggcgccgtttccactacgatatcaagattttcaatcaatggttgaccgaggcagagcagttcctcagaaagacacagatccctgaaaattgggagcatgccaagtacaagtggtatctgaaggagctgcaggacgggatcggacaacgtcagaccgtggtccgcaccctgaacgcaaccggcgaggaaattatccaacagtctagcaagacagatgctagcatcctccaggagaagctcggaagcctcaaccttcgctggcaggaggtgtgcaagcagctgtcagacaggaagaaacgcctggaggaacagaagaacatcctttcagagttccagagagatctgaacgaattcgtgctttggcttgaagaggccgataacatcgcaagcatcccactggagcctggtaaggaacagcaattgaaggagaagctggagcaggtaaagctgctcgtggaagagctgcctctgaggcagggaattctcaagcagctcaacgagactgggggcccggttttggtgagcgccccaattagcccagaagagcaggacaagttggagaacaagctgaagcagaccaacctgcagtggatcaaggtgtcccgcgcactgcccgaaaagcagggagagattgaggcccagatcaaggatctgggacagctggagaaaaagctggaagacctggaggaacagctgaaccatttgctcctgtggttgtcccccatcagaaatcagctggaaatctacaaccaaccgaaccaggaaggtccgtttgatgttaaggaaaccgagatcgctgtgcaggcgaagcagcctgacgtagaagagatcctgtcaaagggacagcacctctacaaggagaagcccgccacccagccagtcaagcggaagctcgaagacctgtccagcgagtggaaagccgtcaaccgcctcctgcaagagctgcgtgccaagcaaccggacctggcgccaggtctgactaccattggcgcaagtcctacccagactgtcaccctggtgacccaacccgttgtgacaaaggaaaccgccatcagcaagctggagatgcctagctctttgatgctggaggttcccgccctcgccgacttcaaccgtgcttggacagaactgaccgactggctgtccctcctggaccaagtgatcaagagccagcgcgtgatggtgggtgatcttgaggatattaacgaaatgattatcaagcagaaggccaccatgcaggacttggagcagaggcgcccccagttggaggaactgatcaccgccgcgcagaatctgaaaaacaaaacttccaatcaggaagcgagaaccatcattacagacagaattgagagaattcaaaaccagtgggacgaagtgcaagaacacctccagaaccggcgtcaacagctgaacgagatgctgaaggattccactcaatggctggaagctaaggaagaggccgagcaggtccttggacaggcacgcgctaagctggagtcctggaaagaaggcccctatacagtggacgcaattcaaaagaaaatcaccgagacgaagcaactggccaaggacctgagacagtggcaaaccaacgttgatgtggctaatgacctggccttgaagctcctgcgtgactactccgcggatgacactagaaaggtgcacatgatcaccgagaatatcaacgctagctggcgctccatccataagcgtgtgagtgagcgcgaagccgctctggaggaaacccatcgcctgcttcaacagtttcctttggacctggaaaagttcctggcctggctcacagaagccgagacaaccgccaacgtgctgcaggacgccacccgtaaggagcgcttgctggaggatagtaagggggtgaaggagttgatgaagcagtggcaagatctgcagggggaaatcgaggcacataccgacgtctatcataacctggatgagaattcccagaaaatcctgcgcagcttggagggcagtgatgacgcggtgcttctgcaacggcgcttggacaacatgaacttcaagtggtccgagttgcgtaaaaagagtctgaacattcgctcccacctggaggcttctagtgaccagtggaagcgcctgcacctgagcctgcaagaactgctcgtgtggctgcaactgaaggatgacgagttgagtcgtcaggcccctatcggaggtgactttccggctgtccagaaacagaacgacgtgcatagggcatttaagcgcgagttgaagacaaaggagccagtcattatgtccacgctggaaaccgtgcgcattttcctgactgagcagcctctggaggggctggagaagctgtaccaggagcctcgcgagttgccccctgaagagcgtgcccagaatgtgacccgcctgttgcgtaagcaggccgaggaagtgaacaccgagtgggagaagctgaacctgcattccgcggattggcagaggaagattgacgagacgctggagaggctccgtgagttgcaggaggcaaccgacgaactggacctcaagctgcgtcaagcagaggtgatcaaggggagttggcagccggtcggcgatttgctgatcgactccctgcaggatcacctggaaaaggttaaagctttgcgcggggaaattgccccgctgaaggagaacgtgagccacgttaacgatctggccagacagctgaccactctgggtatccagctgagcccgtacaatctgagcaccctcgaagacttgaatacgagatggaaactcctgcaggtggcagtggaggacagggtgcgccaattgcacgaggcccaccgggactttggaccggccagccagcatttcctcagtacctctgttcagggcccgtgggagcgggccatctcccccaacaaggtcccctattacatcaaccatgaaacccagactacctgttgggaccaccctaaaatgactgagttgtatcaatctctggccgacttgaataacgtgcgcttctcagcctacaggactgcaatgaaactcagacgcctccaaaaggccctttgtctggacttgctgtcccttagcgctgcctgtgatgctctggatcagcacaatttgaaacaaaacgaccagccgatggacatcctccagattatcaactgcctgactaccatctacgacagactggaacaggagcataataacctggtgaacgtccccctgtgcgtggacatgtgcctgaactggctgctcaatgtgtatgacacgggtagaacaggacggatcagggtcctgtcctttaagaccggtatcatttctctgtgtaaggcacatctggaggacaagtaccgctacctgtttaagcaggtcgccagctccactggcttctgcgatcaaaggcggctgggtctgctcctgcacgactccattcagatcccgagacagttgggtgaagtggcgtccttcggcggttctaatatcgagcctagcgtccgcagctgtttccaattcgccaataacaagccggagatcgaagcggcccttttcctggactggatgcgtctggaacctcagagcatggtttggcttccggtgcttcatcgcgtggccgctgccgaaaccgctaaacatcaggccaaatgtaacatctgtaaggaatgcccgattatcggtttccggtaccgtagcctcaagcatttcaactatgatatttgtcaatcttgcttctttagcggacgggtggccaaaggccacaaaatgcactacccgatggtcgagtattgcactcctacaacctccggcgaggacgtgcgcgattttgccaaagtgctgaaaaacaagtttagaaccaagcgttactttgccaagcatccacgtatgggctacctgcctgtccagaccgttctggaaggtgataacatggagacacccgtgactttgattaacttttggccagtagactctgctccggcaagctcccctcagctctcccacgatgacactcacagccgtatcgagcactacgcctctcgcctggctgagatggagaactctaacggttcatacctgaatgacagtatctccccaaacgagagtatcgatgacgaacatctgctcattcagcattactgtcaatccttgaatcaggacagccccctgtcccagccacggtccccggcccagatcctgatttcactggagtccgaagagagaggggaactggaaaggatcctcgctgacctggaagaggaaaaccggaacctgcaggctgaatacgaccgcttgaagcagcaacacgagcacaagggcttgagcccgttgccaagccctccggagatgatgccaactagccctcagagcccacgggatgccgagctcatcgctgaagctaagttgctgagacagcacaagggacgtctggaggcccgcatgcagatcctggaggatcataataaacagttggagagccaactgcataggctgcgccagctcctggagcagccacaggccgaggctaaggtcaacggaactaccgtttccagcccgtccacatctctgcagcgctcagatagttcccagcctatgttgctgagagtggttggatctcagacctccgactcaatgggcgaagaggacttgctgtctcccccacaggacactagcacaggactcgaagaggtgatggaacagctcaataactcttttccttctagtcggggtcgcaatacccctggaaagcccatgcgcgaagacaccatgtaatag

SEQ ID NO: 20 - DNA, 11061 bp, synthetic construct
  source 1..11061: mol_type = other DNA; note = DMD-5
atgctgtggtgggaggaagttgaagattgttatgagagagaagatgtccagaaaaagacctttaccaaatgggtgaacgcacagttctccaaatttgggaaacagcatatagaaaatttgttcagcgatcttcaggatgggcgcaggttgcttgaccttctggaaggactgaccgggcagaagctgcccaaagagaaagggtctaccagagttcacgctctgaacaacgtgaataaggcactgcgagtcctgcaaaataacaacgtcgacctggtgaacataggcagcactgatattgttgatgggaaccataaactcacactgggtctgatttggaatattatactgcactggcaggtcaaaaatgttatgaaaaatattatggccgggcttcaacagaccaattccgagaagatcctgctgtcttgggttaggcagtccacgaggaactaccctcaagtgaacgttattaacttcacaacatcatggagtgatggacttgccctgaacgccttgatccactcccataggccagacctgttcgattggaatagcgtagtctgtcagcagagtgctacacaacgcctggaacacgcctttaacatagccaggtatcagcttggaattgagaaactcctcgaccctgaagacgtagacaccacttaccccgacaaaaaatccattcttatgtacataacttccttgtttcaggttctcccccagcaggtgtccatcgaggctatacaggaggtcgaaatgctccccagaccccctaaggtcacgaaggaagagcacttccaactgcaccatcagatgcactacagtcaacagatcactgtgagtctcgcacaggggtacgaaaggaccagctcccctaaaccccgcttcaagtcctacgcctatactcaggcagcttacgtaaccacaagtgaccccacgcggtcacccttcccaagccagcacctggaggctcccgaggacaaaagctttggatcatccctgatggaatctgaagtcaatctcgataggtaccaaaccgctctggaggaggtcctcagttggctcctttcagctgaagatactctgcaggcacagggtgagatcagcaatgacgtggaggtagtgaaagatcagtttcacactcacgagggctatatgatggacctgacagcccatcaaggtcgcgttggcaatatcctgcagttggggtccaaactgattggtaccggtaagttgtccgaggacgaagagacagaagtgcaggagcagatgaacctgcttaattctagatgggaatgcctccgcgtggcctctatggagaagcagtccaatctccatcgggttctcatggacttgcagaatcagaagctgaaggaactcaacgactggttgaccaagactgaggagcgcaccagaaagatggaggaggagccactcggtcctgatcttgaagatctgaaacgacaagttcagcagcacaaggtcctgcaggaggatctcgaacaggagcaggtccgggtgaacagcctgacccatatggttgtcgttgtcgacgagtcaagtggggaccatgctacggctgcgcttgaggaacagctgaaggtcttgggcgatagatgggcgaatatctgccgctggactgaagatagatgggtgctgctgcaggatattcttttgaagtggcagcggttgaccgaagaacaatgcttgtttagcgcctggctgagtgagaaagaggatgcggtgaataagatacacactactggcttcaaagaccagaatgaaatgcttagcagcctgcagaaacttgcggttcttaaggccgacttggaaaagaaaaagcagtcaatgggtaagctgtactcactcaagcaggacctgctgtcaactttgaagaacaagagcgtcacgcaaaagactgaggcctggctcgacaattttgctaggtgctgggataacctggtgcagaagttggaaaagtccactgctcagatctctcaagccgtgactactacccagcccagcctgacacagactaccgtcatggaaaccgttaccactgtgacgactagggagcagatactcgttaagcacgctcaagaggaattgccgccacctcctccccaaaagaaacgccagattaccgtcgactctgaaatacgcaagagactggatgtggacatcacggaactgcactcttggattaccagaagcgaggcagtcctccagagccccgagtttgcgatcttcaggaaggaaggcaacttttcagatctgaaggaaaaggtgaacgccatcgagcgcgagaaagccgagaagttccgcaaactgcaggatgcgagccggtctgctcaggccttggtggagcaaatggtgaatgaaggcgtaaatgcggacagcatcaagcaggcctccgagcagctcaattcaagatggatcgagttctgtcagctgctcagcgagcgattgaactggctggagtatcaaaataacataatcgcgttttacaaccagctgcaacagctggaacagatgacgactactgcagagaactggcttaaaatccagcctacaactccttctgaacccacagcaatcaaaagccagctcaagatctgcaaggacgaagtcaaccgcctctccgatttgcagcctcagatagaacgactcaagatccaaagtatcgcactcaaagaaaaggggcaaggccctatgtttctcgatgctgattttgtggctttcaccaaccattttaagcaagtgttttcagatgtgcaagcccgggaaaaggaactgcaaaccatttttgataccctgcctccaatgcggtaccaggagactatgtctgcaattcgcacctgggtgcaacaaagcgagacgaaactttctattccacaactgtccgtgacagactacgaaattatggaacagcgactgggagaactccaagcccttcagtccagccttcaggagcagcagtcaggattgtactatctgtcaaccaccgtgaaggaaatgtcaaaaaaggctccctccgaaatatctcgcaagtatcagtctgagtttgaagagattgaagggagatggaagaaactgtcctcacagctggttgagcactgtcagaagttggaagagcagatgaacaagttgcgcaagattcaaaatcacatacagacactcaaaaagtggatggcggaggttgacgtcttccttaaagaggagtggccagccctgggcgactcagagatcctgaaaaagcaactgaaacagtgccgactgctggtgtccgacatccagacaatacagccaagtttgaatagcgtgaatgagggtggacaaaaaatcaaaaacgaggctgagcccgagttcgcctccagattggaaaccgagctgaaggaacttaatacacagtgggatcatatgtgtcagcaggtttatgcccggaaggaagcactgaaaggggggctggagaaaaccgtgagccttcagaaggaccttagcgagatgcacgagtggatgacccaggcggaggaagagtatctcgaacgggattttgagtataagacaccagatgaacttcagaaggcagtcgaagagatgaaacgggcaaaggaagaggctcagcagaaggaagcaaaggttaaacttctgacagaatccgttaacagcgttatagcccaagctccccccgttgcccaggaagctcttaaaaaggaactggaaacactgacaacaaactaccagtggctgtgcacccgcttgaacggcaaatgcaaaaccctggaagaggtctgggcttgttggcatgaactcctgtcatacctggaaaaagctaataagtggctgaatgaggtggagtttaaattgaaaacgaccgaaaacatccctggcggtgcagaggagatatctgaggtgctcgatagcctggagaatcttatgaggcatagcgaggataaccccaaccagattaggatactggcccaaactctgaccgacggcggagtgatggacgagctgataaacgaggaactggaaacctttaattcacgctggcgagaacttcacgaggaggcagtcaggcgccagaaactgctggagcagtccatccagagtgcccaggagactgagaagtcacttcatctgatccaggagagtttgacgtttatcgacaagcagcttgccgcctacattgcagacaaggtcgatgctgctcagatgccgcaagaggctcagaaaatccagagcgacctgacaagccacgagattagcctggaggaaatgaaaaagcacaaccagggtaaagaggccgcccagcgggttctgagccagatcgatgtcgcacagaagaaactccaagatgtaagcatgaagttccgactgtttcagaaaccagcgaactttgagcagcgactgcaggagtctaaaatgatcctcgatgaggtgaaaatgcatctgccagccctggaaactaagagcgtggagcaggaggtggttcagagtcagctgaaccactgcgtaaacctctataaaagtcttagtgaagtgaagagcgaggtggagatggtgattaagactggtagacagattgtacagaaaaagcagacggagaaccccaaagagttggatgaacgggtgacagcccttaagctccactataacgagctgggagccaaagtgactgagagaaagcagcaacttgaaaaatgtctgaagctgagcagaaagatgcggaaggaaatgaatgtgctgacagagtggctggctgcgaccgacatggaacttacaaagaggagcgccgtggaaggcatgccttccaatctggatagtgaggtcgcttggggcaaagctacacagaaggaaattgagaagcaaaaagtgcatcttaagagcattactgaagttggcgaggctttgaaaaccgtcctcggcaagaaagagactctggtagaagataaactctcactgctgaactcaaactggatcgcagtcactagtcgggccgaagagtggctgaatttgctgttggagtaccaaaagcatatggagactttcgatcagaatgtcgaccacattacgaagtggataatccaggcggacacactgttggatgagtccgagaaaaagaaacctcagcagaaggaagacgtgctcaaacgactcaaagccgaactgaacgacatcaggcccaaagttgattccacccgggatcaagcggccaatctcatggccaaccggggggatcactgcagaaagttggtagaacctcagataagcgaacttaatcatagatttgccgccatatcacataggattaagaccggtaaggcttcaatccccctgaaggaactcgaacagtttaatagtgacatccagaagctcctggagcctctcgaagccgagattcaacagggtgtaaaccttaaggaagaggattttaataaggatatgaatgaggacaatgaaggcacggtcaaagagctcctccagaggggagacaatttgcagcagcggatcaccgatgaacgcaaacgcgaagagatcaaaattaagcagcagctcctccaaacaaagcacaatgcccttaaagacctcagaagccagcggcggaaaaaggctctggaaatcagccatcaatggtatcagtacaagcggcaagcggacgatctcctgaaatgtctggatgatatcgagaaaaaactggccagtctgccagagcccagagatgagcgcaaaatcaaggaaatcgacagggagcttcagaagaaaaaagaggagctgaacgccgtccgaaggcaggccgaagggctgtcagaagatggggccgccatggcagtggaacctacccagattcaactgtccaagcgctggagagagattgaaagtaagttcgcacaattccggaggcttaattttgctcagattcacacagttagggaggaaaccatgatggtgatgaccgaagacatgccactcgaaatcagctacgtccctagcacatatctcacggagattacccacgtgtcccaggcactgctggaagtcgagcaattgttgaacgcccctgatctctgcgccaaggatttcgaagacttgttcaagcaagaggaaagcctcaagaacatcaaagacagtcttcagcagtcctcaggcagaattgatattatccactccaagaaaacagccgctctccagagcgctacccctgtcgaacgagtgaaactgcaagaggccttgtctcagttggacttccagtgggagaaagtcaacaagatgtacaaggacaggcagggtcggttcgaccggagcgtcgaaaaatggcggagattccattatgacatcaagatattcaatcagtggcttacagaggcggagcaatttttgagaaagacgcagattcccgaaaactgggagcatgccaagtacaagtggtacctcaaagagctccaggatggaatcggccagagacagaccgtagttagaacactgaacgcaactggagaagagattatccagcagtcctccaaaactgacgcctctatcctccaggagaagcttggcagccttaacctgcggtggcaggaggtgtgtaaacaactgtcagataggaaaaaaagacttgaggaacagaagaacattctgagcgagttccaacgagatctcaacgagttcgtactgtggctggaggaggccgacaacatcgcttcaatccccctggaacccggcaaggaacagcagcttaaggaaaaattggaacaggtaaaactgttggtagaggagctcccattgcggcagggcattctcaagcagctgaatgaaactggaggtcccgttctggtcagtgcgcccataagcccggaagagcaggacaagttggaaaacaagctgaagcaaacaaatctgcagtggatcaaagtgagccgggctcttccagaaaagcaaggcgagattgaagcccagattaaggatcttggccagcttgagaaaaagctggaggatctggaagagcagctgaaccatttgcttctgtggctgagtcccattagaaaccagctggaaatctacaaccagccaaaccaagagggccctttcgacgtcaaggaaactgagatagcagtccaggcaaaacagcccgatgtcgaagaaatcttgtctaaggggcagcacctctacaaagagaaacctgccacccagcccgtgaagagaaaactggaagacctctcttcagagtggaaggctgtaaacaggttgctccaggagctgagagctaagcaacctgacctcgcacccggcctgactactattggcgccagcccgacccaaaccgtcacactggtgacccagcctgtagtcacaaaagaaaccgccatctccaaactggagatgccttcaagcctgatgctggaggtgcctgctctggccgatttcaacagagcctggacagagctgacagactggctgtctctgctcgaccaagtgataaagtcacagcgcgtcatggtgggtgacctggaggacatcaatgagatgatcataaagcaaaaggctaccatgcaggacttggagcagcgcaggccgcagttggaagaactgattacagcagcccagaacctcaagaacaagacttcaaatcaggaggcaaggaccatcataacagatagaatcgagaggattcagaaccaatgggatgaggtgcaggagcacctgcagaacagaaggcagcagctgaacgaaatgctgaaagactccacacagtggctggaggccaaagaggaggcagaacaggtgctgggccaggctagggccaagctggaaagttggaaggaagggccctacaccgtggacgccatacagaagaaaataacggagacaaaacagctggccaaagacctccgccaatggcagaccaacgtagacgtcgccaatgacctggcactcaagttgttgagagattattccgcagatgacacccgaaaagtacacatgatcactgagaatatcaacgccagttggcggagcattcacaaacgggtgagcgagagagaagccgcccttgaggagacgcatcgccttttgcagcaatttccgcttgacctggaaaagtttctggcttggttgacagaggccgagacaactgctaacgtgctgcaagatgccacccgcaaagagcggcttcttgaggactctaaaggggtgaaagaattgatgaagcagtggcaggacctccagggcgaaatcgaggcccatactgatgtctaccacaacctggatgaaaatagtcagaaaatcctgagatccctggaagggagtgatgatgcggtgctgctgcagcgccgactcgataacatgaactttaagtggtctgaactccggaaaaaaagcctgaacattcggagccatctggaggcttcttctgatcagtggaagagacttcatctgtccttgcaggagcttctggtgtggctgcagctgaaagacgacgagctgtcaaggcaggcaccaatagggggcgacttccccgctgttcagaaacaaaatgatgtgcacagggcattcaagcgggaactgaagacgaaagaaccagtgattatgagcactctggagacggtacgcatctttctgaccgagcagccacttgaaggcctcgaaaagttgtaccaggagccacgggaattgcctcccgaagaacgggctcagaatgttacccgactgcttcgcaagcaggctgaggaagtgaacactgagtgggaaaaactgaatctgcacagtgccgattggcagcgcaaaattgacgagacattggagcgcttgcgggagctgcaggaagccacagatgaacttgatctgaaactcagacaggccgaggtcataaagggttcttggcaacccgtaggcgaccttttgattgactcactgcaggatcatctggagaaagtaaaggcgctgcggggggaaattgctccgctgaaggaaaacgtgagccatgtgaatgatctggctcggcaattgactaccctgggtatccagctgtccccctacaatctgagcactctcgaagacctcaacacaagatggaaactgctgcaggtcgccgtggaggacagggtgaggcagctgcatgaggcacacagggacttcggtccggcaagccaacacttcctgtctacttctgttcaagggccttgggaacgcgctatttccccaaacaaagtgccgtactatattaaccatgaaactcagacaacctgctgggatcatcctaaaatgactgagctgtaccagagcctcgccgatctgaataacgtgaggtttagcgcatacagaaccgcaatgaagctcaggagactgcagaaggcactttgcctggacctcctgagcctctccgctgcctgcgatgccctcgaccagcacaacctgaagcagaatgatcagcctatggacatcctgcaaatcataaattgtctgacgacaatctacgaccggctcgaacaggagcacaataatctcgtgaacgttccactttgtgtggatatgtgtctcaactggctgttgaacgtctatgacaccgggagaactggaaggatccgcgttctgtcctttaagactggaatcatcagcttgtgtaaggcgcaccttgaggacaaatatcgatacctgtttaagcaggttgcaagctccactggattctgcgaccagaggcgcctcgggctgctgcttcatgatagtatccaaatcccacgccaactcggagaggtggcaagcttcggcgggagcaatattgagccaagcgtcagatcttgtttccagtttgccaacaataagcctgagatcgaagccgcactgtttttggattggatgcggttggagccccagagtatggtgtggttgcccgtgctgcatagggtcgctgccgctgaaaccgccaaacaccaggccaagtgcaatatttgcaaggaatgtcccatcatcgggtttagataccggagcctgaagcacttcaattatgatatctgccagtcttgttttttttccggccgagttgctaagggacacaagatgcattacccaatggtggaatattgtacgccaaccaccagcggcgaagatgtcagagattttgcaaaagtactcaagaacaagttccgcaccaagcgatatttcgcgaagcacccccggatgggctacctcccagtgcagacggttctggaaggcgataacatggaaactccagtcaccctgatcaatttctggcctgtggactctgcgcctgcctcttcacctcagctctcccacgatgacacccattccagaatcgagcactacgcgtctcgactggcggagatggagaacagcaatggttcttacctcaatgacagcattagccccaacgaaagcatcgatgacgagcacctcttgattcaacactactgccagtccctgaatcaggacagtcctctgtcccaacccaggagcccagcccagatactgatctctctcgaatcagaagaaagaggggagttggagcgaattttggccgacctggaggaggagaatagaaatcttcaggccgagtacgatagactgaagcagcagcacgagcacaagggcctttctccactgccctctccaccggaaatgatgccaacctcccctcaaagtcccagggacgctgagctcattgccgaggcaaagctgttgcgccagcacaaaggacgcctggaggcgagaatgcagatcctggaagaccataacaagcagcttgagtctcagctgcaccgacttagacagctgttggagcagccacaagcggaagcaaaggtcaatggaactaccgtgagttcccccagtacaagtctgcagcgctctgactcaagccagccaatgctgctccgggtagtcggttctcagactagtgattcaatgggggaggaggatcttctttccccaccccaagacacctcaacagggcttgaggaagtgatggagcagctgaacaactccttcccctcttcacgggggagaaatactcctgggaagcctatgagagaggataccatgtaatga

SEQ ID NO: 21 - DNA, 12706 bp, synthetic construct
  source 1..12706: mol_type = other DNA; note = CBA-hBGi-DMD-1-WPRE3-DTS
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggattgctacgagcgggaggatgtgcagaaaaagactttcaccaagtgggtaaatgctcagttctctaagttcgggaagcaacacatcgagaacttgttttccgacctccaggatggcaggcgcctgcttgacttgctggagggtctgactggtcagaagctccctaaggagaagggcagcaccagagttcacgctctgaataacgttaacaaagccctgagggtcctccagaataacaatgtagacctggtgaacatcggttctaccgacatcgtggacgggaatcataaactgaccttggggctcatctggaacattatcttgcactggcaggtgaagaacgtcatgaagaatatcatggccgggctgcaacagaccaactccgaaaagattttgctcagctgggtgcgccagtccacccgcaattatccacaagtcaacgttatcaatttcaccactagttggagtgatggcctcgccctgaacgccttgatccacagtcaccgccccgacctgtttgactggaactctgtggtttgccaacagagcgctacccagcgtctcgaacatgctttcaatatcgctagataccagctcggtatcgagaagctgttggatcctgaggacgtagacacgacctaccccgataaaaagagcatcctgatgtatatcacttctctgtttcaggtgcttcctcaacaggtgtccattgaagccatccaagaggtcgaaatgctgcccaggccccctaaggtgacaaaagaagagcatttccagctgcatcaccagatgcattacagtcaacagattaccgtcagcttggctcagggatatgagcgcacgtcctctcctaagcccagatttaagtcctacgcatatacccaggctgcctacgtgaccacttccgacccaactaggagcccctttccctcccaacacctggaggcccctgaggacaaaagcttcggatctagtctgatggagtccgaggtgaatctggatcggtaccaaactgctctggaggaagtgctgtcctggcttctgtccgctgaagacaccctgcaggcgcaaggtgagatttctaacgacgtcgaggtcgtgaaggatcagttccacactcatgagggctacatgatggacttgacagctcaccagggaagagttggtaacatcctgcagcttggctccaagttgattggcacgggaaagcttagcgaggacgaggaaaccgaggtacaggagcagatgaacttgctgaattcccgctgggagtgcctgagagttgcgtccatggaaaagcagtcaaaccttcacagagtgctgatggaccttcagaaccagaagctgaaggagctgaacgattggctgacaaagaccgaggaacgcacaagaaaaatggaagaggaacctctgggacctgatctggaggatctgaagcgccaggtccaacagcataaggtgctgcaggaggatttggagcaggagcaggtgcgggtcaacagcctgacacatatggtggttgtggtcgatgagtcctctggcgatcatgctaccgcagcgctggaagagcagctgaaggtgctgggggatcgctgggccaacatttgcagatggacagaagaccggtgggtcctgcttcaagatatcctgctcaagtggcagcgcctgactgaagagcagtgcctgttctcagcttggttgagcgaaaaagaggacgcagttaacaagatccataccacaggcttcaaggatcagaatgagatgctgagctcactgcagaagctggctgtgctgaaggccgacttggagaagaaaaagcagtccatgggcaaactgtatagcctgaagcaggacctgctcagcaccctgaagaacaaaagcgttacgcagaaaactgaggcctggttggacaactttgctcgctgctgggataaccttgtgcagaagctggaaaagtcaaccgcacagatctcccaggctgtgaccactacgcagcctagcttgacccagacaaccgttatggaaaccgtgacaaccgtgacaacgagagagcaaatcctggttaagcatgcccaggaggaactgccgccacccccgcctcagaagaaacggcagattacggtggacagcgagatccgcaagaggctggacgtcgacatcactgagctccactcatggatcacgcggagtgaggccgtgctgcagtctcccgagttcgctattttccgcaaggagggaaacttctctgacttgaaagagaaggtgaatgccatcgagcgtgaaaaggcagaaaagttcagaaagctgcaggacgcctctcgctctgcgcaggccctggtggagcagatggtcaacgagggtgttaacgctgacagcatcaaacaggcaagcgaacagctgaactccagatggatcgaattttgccaactgctctccgaacgcctcaactggttggagtaccagaacaatattatcgctttttacaaccagctgcagcaactggagcagatgacaactaccgccgaaaactggctgaaaattcagccaaccacaccgtccgagccaactgcgatcaagtctcagctgaagatctgcaaggacgaagtgaaccgtcttagcgacttgcaaccccagattgaacggctcaagattcagtctattgctctgaaggagaagggtcagggtccgatgttcctggatgcagattttgtcgcatttaccaatcacttcaaacaggtgtttagtgacgtccaggcgcgcgagaaagagctgcagactatttttgacaccctgcctcccatgaggtaccaggagacaatgagtgccattcgcacctgggtgcaacagtccgagacgaaattgtcaatcccacagctgtccgtgacagactacgagattatggagcaacggttgggggagctgcaggctcttcagagttccctgcaggagcaacagagcggactgtactatctttccactaccgtgaaggaaatgagtaagaaagccccttccgagatcagccgcaaataccagtctgaattcgaagagatcgagggacgctggaaaaagctgtcaagccagctggtcgagcactgtcagaagctggaagagcagatgaacaagctgcgtaaaatccagaaccatattcagaccctcaagaaatggatggcagaagttgacgtgtttctgaaggaagagtggcctgccctcggcgatagcgaaatccttaagaaacaactcaagcagtgccgcctcctggtgtccgatattcagacaatccagccttccctgaacagcgttaatgagggcggacagaaaattaaaaacgaggccgaacccgagttcgcttctcgcctggagactgaactgaaggagctgaatacccagtgggaccatatgtgtcaacaggtctacgcacgcaaagaggcactgaagggcggtctggagaagactgtctcactccagaaggacctgagcgagatgcacgagtggatgacccaggccgaagaggaatacctggagcgtgattttgagtataagaccccagacgaactccagaaagccgttgaagagatgaagagggctaaggaggaagcccaacagaaggaggcgaaagtgaagctcctgacagagtccgtgaactccgtcatcgctcaagctcctcccgtggctcaggaggccctcaagaaagagctggaaactctgaccacaaattaccagtggttgtgtacccgcttgaacggtaagtgcaagaccctggaagaggtgtgggcctgctggcacgagctcctgtcctatcttgagaaggcgaacaaatggctgaacgaagtggaattcaagttgaaaacaactgagaacattccgggaggcgctgaggaaatctctgaggtgctggactctctggaaaatctgatgcgtcattctgaagacaatcctaaccagatcaggattctggcacagaccctcactgacggaggcgttatggacgaacttatcaacgaggaactggagacattcaattcccgttggcgtgagctgcacgaagaggccgtgcgccgtcagaagttgctggaacagtctatccagtccgcccaggagacagagaaatctctccacctgatccaggagagtctgacgtttatcgataagcagctggctgcctacatcgccgacaaggtggacgctgcccagatgccccaagaggctcagaagatccagtccgacctgacgtctcatgaaatcagcctggaagagatgaaaaagcacaaccagggcaaagaggctgcccagcgcgtcctctcccagattgacgtggctcagaaaaagttgcaggacgtgagcatgaagtttaggctgttccagaaacccgccaacttcgaacagagactgcaggagtctaagatgatcctggatgaggtcaagatgcacctccctgctctggaaacaaaatctgtggagcaggaggtcgtgcagtcacaactgaaccattgtgtgaacctctacaagtctctcagcgaggtgaagtccgaggtcgagatggtgatcaagaccggacgccagattgtgcagaaaaagcagacagagaacccaaaggagctggacgaacgggtgaccgccctgaagctccactacaacgagttgggtgccaaagtcactgaaagaaagcaacagctggagaagtgtctgaagctgagccgcaagatgcgcaaagaaatgaacgtcttgaccgagtggttggcggccaccgacatggagctgaccaagagatccgctgtggagggcatgccctccaaccttgactcagaggtggcctggggcaaggccacacagaaggagatcgaaaaacagaaggtgcatcttaaatcaatcactgaggtgggcgaagctctgaagacagtgctgggcaagaaagagacacttgtagaggacaagctctcccttctgaatagcaattggatcgccgtaactagtcgcgcggaggaatggctgaacctcctgttggaataccagaaacacatggaaaccttcgatcagaacgtggaccatattaccaagtggattatccaggctgacactctcctggacgagtctgagaaaaagaaaccccagcaaaaggaagatgtgctgaagcgcctgaaggcggaattgaacgacatccgcccgaaggttgattccacccgcgaccaagcggccaaccttatggctaaccgcggggaccattgcagaaaactggtggaaccgcagatctctgagctgaaccacagattcgccgcgatttctcacagaattaagactggcaaggcctccatccctctgaaagagctggagcagttcaactctgatattcagaagcttctggagcccctggaggcagaaatccaacagggcgtcaacctgaaagaggaagactttaataaggacatgaatgaggataacgaaggtaccgtcaaggagctcctgcagaggggcgacaatctgcagcaaagaatcactgacgaacgtaagcgcgaagagattaagatcaagcaacagctgttgcagactaagcacaacgccctgaaggatctgaggtcccagcgcaggaaaaaggccctggagatttcccaccagtggtaccagtacaagcgccaggccgatgaccttctgaagtgcctcgacgatatcgagaaaaagctggcctcactgcccgagccccgcgacgagcgcaagatcaaggagattgaccgtgagctccagaagaaaaaggaggaactgaacgcggtgcgcagacaggccgagggcctctccgaggacggcgctgcgatggctgttgagcccactcagatccaactgagcaagcggtggagagagatcgagtctaagttcgcgcaatttaggcgcctcaacttcgcccagattcacacggtccgtgaggaaacgatgatggtgatgacagaggatatgccactggaaatcagctacgtgcccagtacatatctcaccgaaatcacccatgtgtcccaggctctcctggaggtggaacagttgctcaacgctcccgacttgtgcgctaaggacttcgaggacctgttcaagcaggaagagagcctgaaaaacattaaggactccctgcaacagtcaagcggccgcatcgatatcattcattctaagaaaaccgctgccctgcagtctgcaacccccgtagagcgcgtcaaactgcaggaagctctcagccaactggatttccagtgggagaaggtgaacaagatgtataaggatcgtcagggccggttcgaccgctctgtggagaagtggcgccgtttccactacgatatcaagattttcaatcaatggttgaccgaggcagagcagttcctcagaaagacacagatccctgaaaattgggagcatgccaagtacaagtggtatctgaaggagctgcaggacgggatcggacaacgtcagaccgtggtccgcaccctgaacgcaaccggcgaggaaattatccaacagtctagcaagacagatgctagcatcctccaggagaagctcggaagcctcaaccttcgctggcaggaggtgtgcaagcagctgtcagacaggaagaaacgcctggaggaacagaagaacatcctttcagagttccagagagatctgaacgaattcgtgctttggcttgaagaggccgataacatcgcaagcatcccactggagcctggtaaggaacagcaattgaaggagaagctggagcaggtaaagctgctcgtggaagagctgcctctgaggcagggaattctcaagcagctcaacgagactgggggcccggttttggtgagcgccccaattagcccagaagagcaggacaagttggagaacaagctgaagcagaccaacctgcagtggatcaaggtgtcccgcgcactgcccgaaaagcagggagagattgaggcccagatcaaggatctgggacagctggagaaaaagctggaagacctggaggaacagctgaaccatttgctcctgtggttgtcccccatcagaaatcagctggaaatctacaaccaaccgaaccaggaaggtccgtttgatgttaaggaaaccgagatcgctgtgcaggcgaagcagcctgacgtagaagagatcctgtcaaagggacagcacctctacaaggagaagcccgccacccagccagtcaagcggaagctcgaagacctgtccagcgagtggaaagccgtcaaccgcctcctgcaagagctgcgtgccaagcaaccggacctggcgccaggtctgactaccattggcgcaagtcctacccagactgtcaccctggtgacccaacccgttgtgacaaaggaaaccgccatcagcaagctggagatgcctagctctttgatgctggaggttcccgccctcgccgacttcaaccgtgcttggacagaactgaccgactggctgtccctcctggaccaagtgatcaagagccagcgcgtgatggtgggtgatcttgaggatattaacgaaatgattatcaagcagaaggccaccatgcaggacttggagcagaggcgcccccagttggaggaactgatcaccgccgcgcagaatctgaaaaacaaaacttccaatcaggaagcgagaaccatcattacagacagaattgagagaattcaaaaccagtgggacgaagtgcaagaacacctccagaaccggcgtcaacagctgaacgagatgctgaaggattccactcaatggctggaagctaaggaagaggccgagcaggtccttggacaggcacgcgctaagctggagtcctggaaagaaggcccctatacagtggacgcaattcaaaagaaaatcaccgagacgaagcaactggccaaggacctgagacagtggcaaaccaacgttgatgtggctaatgacctggccttgaagctcctgcgtgactactccgcggatgacactagaaaggtgcacatgatcaccgagaatatcaacgctagctggcgctccatccataagcgtgtgagtgagcgcgaagccgctctggaggaaacccatcgcctgcttcaacagtttcctttggacctggaaaagttcctggcctggctcacagaagccgagacaaccgccaacgtgctgcaggacgccacccgtaaggagcgcttgctggaggatagtaagggggtgaaggagttgatgaagcagtggcaagatctgcagggggaaatcgaggcacataccgacgtctatcataacctggatgagaattcccagaaaatcctgcgcagcttggagggcagtgatgacgcggtgcttctgcaacggcgcttggacaacatgaacttcaagtggtccgagttgcgtaaaaagagtctgaacattcgctcccacctggaggcttctagtgaccagtggaagcgcctgcacctgagcctgcaagaactgctcgtgtggctgcaactgaaggatgacgagttgagtcgtcaggcccctatcggaggtgactttccggctgtccagaaacagaacgacgtgcatagggcatttaagcgcgagttgaagacaaaggagccagtcattatgtccacgctggaaaccgtgcgcattttcctgactgagcagcctctggaggggctggagaagctgtaccaggagcctcgcgagttgccccctgaagagcgtgcccagaatgtgacccgcctgttgcgtaagcaggccgaggaagtgaacaccgagtgggagaagctgaacctgcattccgcggattggcagaggaagattgacgagacgctggagaggctccgtgagttgcaggaggcaaccgacgaactggacctcaagctgcgtcaagcagaggtgatcaaggggagttggcagccggtcggcgatttgctgatcgactccctgcaggatcacctggaaaaggttaaagctttgcgcggggaaattgccccgctgaaggagaacgtgagccacgttaacgatctggccagacagctgaccactctgggtatccagctgagcccgtacaatctgagcaccctcgaagacttgaatacgagatggaaactcctgcaggtggcagtggaggacagggtgcgccaattgcacgaggcccaccgggactttggaccggccagccagcatttcctcagtacctctgttcagggcccgtgggagcgggccatctcccccaacaaggtcccctattacatcaaccatgaaacccagactacctgttgggaccaccctaaaatgactgagttgtatcaatctctggccgacttgaataacgtgcgcttctcagcctacaggactgcaatgaaactcagacgcctccaaaaggccctttgtctggacttgctgtcccttagcgctgcctgtgatgctctggatcagcacaatttgaaacaaaacgaccagccgatggacatcctccagattatcaactgcctgactaccatctacgacagactggaacaggagcataataacctggtgaacgtccccctgtgcgtggacatgtgcctgaactggctgctcaatgtgtatgacacgggtagaacaggacggatcagggtcctgtcctttaagaccggtatcatttctctgtgtaaggcacatctggaggacaagtaccgctacctgtttaagcaggtcgccagctccactggcttctgcgatcaaaggcggctgggtctgctcctgcacgactccattcagatcccgagacagttgggtgaagtggcgtccttcggcggttctaatatcgagcctagcgtccgcagctgtttccaattcgccaataacaagccggagatcgaagcggcccttttcctggactggatgcgtctggaacctcagagcatggtttggcttccggtgcttcatcgcgtggccgctgccgaaaccgctaaacatcaggccaaatgtaacatctgtaaggaatgcccgattatcggtttccggtaccgtagcctcaagcatttcaactatgatatttgtcaatcttgcttctttagcggacgggtggccaaaggccacaaaatgcactacccgatggtcgagtattgcactcctacaacctccggcgaggacgtgcgcgattttgccaaagtgctgaaaaacaagtttagaaccaagcgttactttgccaagcatccacgtatgggctacctgcctgtccagaccgttctggaaggtgataacatggagacacccgtgactttgattaacttttggccagtagactctgctccggcaagctcccctcagctctcccacgatgacactcacagccgtatcgagcactacgcctctcgcctggctgagatggagaactctaacggttcatacctgaatgacagtatctccccaaacgagagtatcgatgacgaacatctgctcattcagcattactgtcaatccttgaatcaggacagccccctgtcccagccacggtccccggcccagatcctgatttcactggagtccgaagagagaggggaactggaaaggatcctcgctgacctggaagaggaaaaccggaacctgcaggctgaatacgaccgcttgaagcagcaacacgagcacaagggcttgagcccgttgccaagccctccggagatgatgccaactagccctcagagcccacgggatgccgagctcatcgctgaagctaagttgctgagacagcacaagggacgtctggaggcccgcatgcagatcctggaggatcataataaacagttggagagccaactgcataggctgcgccagctcctggagcagccacaggccgaggctaaggtcaacggaactaccgtttccagcccgtccacatctctgcagcgctcagatagttcccagcctatgttgctgagagtggttggatctcagacctccgactcaatgggcgaagaggacttgctgtctcccccacaggacactagcacaggactcgaagaggtgatggaacagctcaataactcttttccttctagtcggggtcgcaatacccctggaaagcccatgcgcgaagacaccatgtaatagctcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 22 - DNA, 12706 bp, synthetic construct
  source 1..12706: mol_type = other DNA; note = CBA-hBGi-DMD-3-WPRE3-DTS
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggactgttacgagagagaggacgtgcagaagaagacattcaccaagtgggtgaacgctcagttcagcaagttcggcaagcagcacatcgagaacctgtttagcgatctacaagatggcagaagactgctggacctgctggagggcctgaccgggcagaagctgcccaaagaaaaggggtctacaagagtgcacgccctgaacaacgtgaacaaggccctgagagtgctgcagaacaacaacgtggacctggtgaacatcggcagcaccgacatcgtggacggcaaccacaagctgaccctgggcctgatctggaacatcatcctgcactggcaagtgaagaacgtgatgaagaacatcatggccggcctgcagcagaccaacagcgagaagatcctgctgagctgggtgagacagagcacaagaaactacccccaagtgaacgtgatcaacttcaccacaagctggagcgacggcctggccctgaacgccctgatccacagccacagacccgacctgttcgactggaacagcgtggtgtgtcagcagagcgccacacagagactggagcacgccttcaacatcgctagatatcagctgggcatcgagaagttactggaccccgaagatgtggacaccacctaccccgacaagaagagcatcctgatgtacatcacaagcctgttccaagtgctgcctcagcaagtgagcatcgaggccatccaagaagtcgagatgctgcctagaccccccaaggtgaccaaggaggagcactttcagctgcaccatcagatgcactactctcagcagatcacagtcagcttggcacaaggctacgagagaacaagcagccccaagcctagattcaagagctacgcctacacccaagccgcctacgtgaccacaagtgaccccacaagaagccccttcccttctcagcacctggaggcccccgaggacaagagcttcggcagcagcctgatggagagcgaggtgaacctggacagatatcagaccgcgttagaagaagtgttgagctggctgctgagcgccgaggacaccctgcaagcccaaggcgagatcagcaacgatgtggaggtggtgaaggatcagttccacacccacgagggctacatgatggacctgaccgcccaccaaggcagagtgggcaacatcctgcagctgggcagcaagctgatcggcaccggcaagctgagcgaggacgaggaaaccgaggttcaagagcaaatgaacctgctaaacagcagatgggagtgcctgagagtggctagcatggaaaagcagagtaacctgcacagagtgctgatggacctgcagaatcagaaattgaaagaacttaatgattggctgaccaagaccgaggagagaacaagaaagatggaggaggagcccctgggccccgacctggaggacctgaagagacaagtgcagcagcacaaggtgctgcaagaggacctggagcaagagcaagtgagagtgaactctctgacacacatggtggtggtagtggacgagagcagcggcgatcacgctaccgcggcgctggaagagcagctgaaagtgctgggcgacagatgggccaacatctgcagatggaccgaggacagatgggtgctgctgcaagacatcctgctgaagtggcagagactgaccgaggagcagtgcctgttcagcgcctggctgagcgagaaggaagacgctgtgaacaagatccacaccaccggcttcaaggatcagaacgagatgctgagctccctccaaaagctcgcagtgctgaaggccgacctggaaaagaaaaaacagtcgatgggcaagctgtacagcctgaagcaagacctgctgagcaccctgaagaacaagagcgtgacacagaagaccgaggcctggctggacaacttcgctagatgctgggacaacctggtacagaagttagagaagtctaccgctcagatcagccaagccgtaaccaccacacaacctagcctgactcagaccaccgtgatggaaaccgtgaccaccgtgacaacccgtgagcagatcctggtgaagcacgcccaagaggagctgcccccccccccccctcagaagaagagacagatcaccgtggactcagagattcgcaagagactggacgtggacatcaccgagctgcacagctggatcacaagaagcgaggccgtgctgcagagccctgagttcgcgatctttagaaaggagggcaacttcagcgacttgaaggagaaagtgaacgccatcgagagagagaaggccgagaagttcagaaagttgcaagacgcctcaagaagcgcccaagccctggtggagcagatggttaacgaaggcgtgaatgccgacagcatcaagcaagctagcgagcagctgaatagccggtggattgaattctgtcagctgctgagcgagagattaaactggctggagtatcagaacaacatcatcgccttctacaatcagctgcagcaactagaacaaatgaccaccaccgccgagaactggctaaagattcagcccaccacccctagcgagcccaccgccatcaaatctcagctgaaaatttgcaaggacgaagtgaacagactgtcggacctgcagcctcaaatcgaaagactgaaaattcagagcatcgcacttaaggagaagggccaaggacccatgttcctggacgccgacttcgtggccttcaccaaccacttcaagcaagtgttcagcgacgtgcaagctagagagaaggagctgcagaccatcttcgacaccctgccccccatgagataccaagaaaccatgagcgccatcagaacctgggtgcagcagagcgaaaccaagctgagcatccctcagctgagcgtgaccgactacgagatcatggagcaaagactgggagagctgcaagccctgcagagcagcctgcaagagcagcagagcggcctgtactacctgagcaccaccgtgaaggagatgtcgaagaaagccccgtcggagatcagcagaaagtatcagagcgagttcgaggagatcgagggcagatggaagaagctgagcagccaactggtggaacactgtcagaaattagaagaacagatgaacaagctgagaaagattcagaaccacattcagaccctgaagaagtggatggccgaggtggacgtgttcctgaaagaggagtggcccgccctgggcgattccgaaatcttaaaaaagcagctgaagcagtgcagactgctggtgtcagatatccaaaccattcaaccaagcctgaacagcgtgaacgaaggcgggcagaagatcaagaacgaggccgagcccgagttcgctagcagactggaaaccgagctgaaggagctgaatacacagtgggaccacatgtgtcagcaagtgtacgctagaaaggaagcgctgaagggcgggctggagaagaccgttagcctgcaaaaggatctgagcgagatgcacgagtggatgacccaagccgaggaggagtacctggagagagacttcgagtacaagacccccgacgagctgcagaaggccgtcgaggaaatgaagagagcgaaggaggaggctcagcagaaggaagccaaggtgaagctgctgactgaatctgtcaacagtgtgatagctcaagcaccccccgttgcccaagaggcactaaagaaggagctggaaactctgaccaccaactatcagtggctgtgcacaagactgaacggcaagtgcaagaccctggaggaagtgtgggcctgctggcacgagctgctgagctacctggagaaggccaacaagtggctgaacgaggtggagttcaagctgaagaccaccgagaacatccccggcggcgccgaggagatcagcgaggtgctggacagcctggagaacctgatgagacacagcgaggacaaccccaatcagatcagaatcctggctcagaccctgaccgacggcggcgtgatggacgagctgatcaacgaggagctggaaacctttaactctagatggcgagaactgcacgaggaggccgtgagaagacagaagctgctggagcaaagcattcagagcgcccaagaaaccgagaagagcctgcacctgatccaagagagcctgaccttcatcgacaagcagctggccgcctacatcgccgacaaggtggacgccgctcagatgccacaagaggctcagaagattcagtccgatctgacaagccacgagatcagcctggaggagatgaaaaaacacaaccaaggcaaggaggccgctcagagagtgctgtctcagatcgacgtggctcagaagaagctgcaagacgtgagcatgaagttcagactgtttcagaagcccgccaacttcgagcagcgactgcaagagagcaagatgatcctggacgaggtgaagatgcacctgcccgccctggaaaccaagagcgtggagcaagaggtggtgcagtctcagttgaaccactgcgtgaacctgtacaagagcctgtcggaggtcaagagtgaggtcgagatggtgatcaaaactgggagacagatcgtgcaaaaaaagcagaccgagaaccccaaggagctggacgagcgtgttaccgccctgaagctgcactacaacgagctgggcgccaaggtgaccgagagaaagcagcagctggagaagtgcctgaagctgagcagaaagatgagaaaggagatgaacgtgctgaccgagtggctggccgccaccgacatggagctgaccaagagaagcgccgtggagggcatgcctagcaacctggacagcgaggtggcctggggcaaggccacacagaaggagatcgagaagcagaaggtgcacctgaagagcatcaccgaggtgggcgaggccctgaagaccgtgctgggcaagaaggaaaccctggtggaggacaagctgagcctgctgaactcgaactggatcgccgtgacaagcagagccgaggagtggctgaacctgctgttagagtaccaaaaacacatggaaaccttcgatcagaacgtggaccacatcaccaagtggatcatccaagccgacaccctgctggacgaaagcgaaaagaagaagccgcaacaaaaggaggatgtgctgaagagactgaaggccgaactgaacgatatcagacccaaggtggacagcacacgggaccaagccgccaacctgatggccaacagaggcgaccactgcagaaaactggtggagcctcagatcagcgagctgaaccacagattcgccgccatcagccacagaatcaaaaccggcaaagcgagcatccccttgaaggaactggagcaatttaacagcgacatccaaaaattgctcgaaccactggaggccgagattcagcaaggcgtgaacctgaaggaagaggacttcaacaaggacatgaacgaggacaacgagggcaccgtgaaagaactgctgcagagaggcgacaacctgcagcagagaatcaccgacgagagaaagagagaggagatcaagatcaagcagcagctgctgcagactaagcacaacgccctgaaggatctgagatctcagaggagaaaaaaggctctggaaatttcacatcagtggtatcagtacaagagacaagcagacgacctgctgaagtgcctggacgacatcgagaagaagttagctagcctgcccgagcctagagacgagagaaagatcaaggagatcgacagagagttacaaaagaagaaggaggagctgaatgcggtaagaagacaagcggaaggcctgtccgaggacggcgcagccatggccgtggagcccacacagattcagctcagcaagagatggagagagatcgagagcaagttcgctcagttcagaagactgaacttcgctcagatccacaccgtgagagaggaaaccatgatggtgatgaccgaggacatgcccctggagatctcatacgttcctagcacctacctgaccgagatcacccacgtgagccaagccctgctggaggtggagcagctgctgaacgcccccgacctgtgcgccaaggacttcgaggacctgttcaaacaagaggagagcctgaagaacatcaaggacagcctgcagcaaagtagcggcagaatcgacatcatccacagcaagaagaccgccgccctgcagagcgccacccccgtggagagagtgaagcttcaagaggccctttcgcagctggattttcagtgggagaaggtgaacaaaatgtacaaggacagacaaggcagattcgacagaagcgtggagaagtggagaagattccactacgacatcaagatcttcaatcagtggctgaccgaggcagagcagttcctgagaaagacacagatccccgagaactgggagcacgccaagtacaagtggtacctgaaggagctgcaagacggcatcgggcagagacagaccgtggtgagaaccctgaacgccaccggcgaggagatcatccaacaaagctccaaaaccgacgctagcatcctgcaagagaaactgggcagcctgaacctgagatggcaagaggtgtgcaagcagctgagcgaccgcaaaaaacggttagaggagcagaagaacatcctgagcgagtttcagcgggacctgaacgagttcgtgctgtggctggaggaggccgataacatcgctagcatccccctggagcccggcaaggagcagcagttaaaagaaaaacttgagcaagtgaagctgctggtggaggagctgcccctgagacaaggcatcctgaagcagctgaacgagactggcggccccgtgctggtgagcgcccccatcagccccgaggagcaagacaagctggagaacaagctgaagcagaccaacctgcagtggatcaaggtgagcagagccctgcccgagaagcaaggcgaaatcgaggcccaaataaaggacctggggcagctggagaagaaactcgaagacttggaagaacaactcaaccacttgctgctgtggctgagccccatcagaaatcagctggagatctacaatcagcccaaccaagaggggccattcgacgtgaaggaaaccgagatcgccgtgcaagccaagcagcccgatgtggaggagatcctgagcaaggggcagcacctgtacaaggagaagcccgccacacagcccgtcaagcgtaaactagaggatctttcctctgagtggaaggccgtgaaccgtctgctacaagagctgcgggctaagcaacccgacttagcgcccggcctgaccaccatcggtgcaagccccacgcagaccgtgaccctggtgacccaacccgtggtgaccaaggaaaccgccatcagcaagctggagatgcctagcagcctgatgctggaggtgcccgccctggccgacttcaacagagcctggaccgagctgaccgactggctgagcctgctggaccaagtgatcaagtctcagagagtgatggtgggcgatctggaggacatcaatgagatgatcatcaagcagaaggccaccatgcaagacctggagcagagaagacctcagctggaggagctgatcaccgccgctcagaacctgaaaaacaagacgagcaaccaagaggctagaaccatcatcaccgacagaatcgagagaattcagaatcagtgggacgaggtgcaagagcacctgcagaacagaagacagcaactaaacgaaatgctcaaggacagcacacagtggctggaggccaaggaggaggcggagcaagtgttgggccaagctagagccaagctggagagctggaaggagggaccctacaccgtggacgccattcagaagaagatcaccgaaaccaagcagctggccaaggacctgcgacagtggcagaccaacgtggacgtggccaacgatcttgccctgaagctgctgcgcgactatagcgccgacgacacaagaaaggtgcacatgatcaccgagaacatcaacgctagctggcgtagcatccacaagagagtgagcgagagagaggcggctttggaggaaacccatagactgctgcagcagtttcccctggacctggagaagttcctggcctggctgaccgaagccgaaacaaccgccaacgtgctgcaagacgccacaagaaaggagagactgctggaggacagcaagggcgtgaaggagctgatgaagcagtggcaagacctgcaaggcgagatcgaagcgcacaccgacgtgtaccacaacctggacgagaactctcagaagatcctgagaagcctggagggcagcgacgacgccgtgctgttacagagaaggctggacaacatgaacttcaagtggagcgagctgagaaagaagagcctgaacatcagaagccacctggaggctagcagcgatcagtggaagagactgcacctgagcctgcaagagctgctggtctggcttcagttgaaggacgatgagctgagcagacaagcccccatcggcggcgacttccccgccgtgcagaagcagaacgacgtgcatagagccttcaagagagagctgaagaccaaggagcccgtgatcatgagcaccctggaaacggtgagaatcttcctgaccgagcagcccctggagggcctggagaagctgtaccaagagcctagagagctgccccccgaggagagagctcagaacgtgacaagactgctgagaaagcaagccgaagaggtgaacaccgagtgggagaagctgaacctgcacagcgccgactggcagagaaagatcgacgaaaccctggagagactgagagaattgcaagaggcgaccgacgagctggacctgaagctgagacaagccgaggtcatcaagggcagctggcagcccgtgggcgatctattaattgactctctacaagaccacctggagaaagtgaaggccctgagaggcgagatcgcccccctgaaggagaacgtgagccacgtgaatgatctggcacgtcagctgaccaccctgggcatacaactgtccccctataacctgagcacactggaggatttgaatactagatggaagctgctgcaagtggccgtggaggacagagtaagacaattacatgaggcccacagagacttcggccccgcttctcagcacttcctgagcacaagcgtgcaaggcccctgggagagagccatttcccccaacaaggtcccctactacatcaaccacgagacacagaccacctgctgggaccaccccaagatgaccgagctgtatcagagcctcgccgatctgaacaatgtgagattcagcgcctacagaaccgccatgaagctgagaagactgcagaaggccctgtgcctggacttattaagcctgagcgccgcctgcgacgccctggatcagcacaacctgaagcaaaacgaccaacccatggacatcctgcagatcatcaactgcctcaccaccatatacgacagactggaacaagagcacaacaacctggtgaacgtgcccctgtgcgtggacatgtgcctgaactggctgctgaacgtgtacgacaccggcagaaccggcagaatcagagtgctgagcttcaagaccggcatcatcagcctgtgcaaggcccacctggaggacaaatacagataccttttcaaacaagtggctagcagcaccggcttctgcgaccaaagaagactgggtctgctgctgcacgacagcattcagatccctagacagctgggcgaggtggctagcttcggcggcagcaacatcgagcctagcgtgagaagctgctttcagttcgccaacaacaagcccgagatagaggcagccctcttcttagactggatgagactggagcctcagagcatggtatggctgccggtcctgcacagagtggccgccgccgaaaccgccaagcaccaagccaagtgcaacatctgcaaggagtgccccatcatcggcttcagatacagaagcctgaagcacttcaactacgacatctgtcagagctgcttcttcagcggcagagtggccaagggccacaagatgcactaccccatggtggagtactgcacccccaccacaagcggcgaggacgtgagagacttcgccaaggtgttgaagaataagtttagaaccaagagatacttcgccaagcaccctagaatgggctacctgcccgtgcagaccgtgctggagggcgacaacatggaaacccccgtgaccctgatcaacttctggcccgtggacagcgcccccgctagcagccctcagctgagccacgacgacacccacagcagaatcgagcactacgctagcagactggccgagatggagaacagcaacggcagctacctgaacgacagcatcagcccgaatgagagcatcgacgacgagcacctgctgattcagcactactgtcagagcctgaaccaagacagccccctgtctcagcctagaagccccgctcagatcctgatcagcctggagagcgaggagagaggcgagctggagagaatcctagcagatctggaggaagagaacagaaacctgcaagccgagtatgaccgactgaaacagcaacatgagcacaaaggcctgagcccactgccctccccccccgagatgatgcccacaagccctcagagccctagagacgccgagctgatcgctgaagcgaagttacttagacaacacaagggcagactggaggctagaatgcagatcctggaggaccacaacaagcagctggagtctcagctgcacagactgaggcagctgctggaacagccccaagccgaggccaaagtgaacggcaccaccgtgagcagtcctagcacaagcctgcagagaagcgacagctctcagcccatgctgctgagagtggtgggctctcagacaagcgacagcatgggcgaggaggatctgctgagtccgccccaagacacaagcaccggcctggaagaggtgatggagcaactgaacaatagcttccctagcagcagaggcagaaacacccccggcaagcccatgagagaggacaccatgtgatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 23 - DNA, 12706 bp, synthetic construct
  source 1..12706: mol_type = other DNA; note = CBA-hBGi-DMD-4-WPRE3-DTS
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 24 - DNA, 12706 bp, synthetic construct
  source 1..12706: mol_type = other DNA; note = CBA-hBGi-DMD-5-WPRE3-DTS
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaggaagttgaagattgttatgagagagaagatgtccagaaaaagacctttaccaaatgggtgaacgcacagttctccaaatttgggaaacagcatatagaaaatttgttcagcgatcttcaggatgggcgcaggttgcttgaccttctggaaggactgaccgggcagaagctgcccaaagagaaagggtctaccagagttcacgctctgaacaacgtgaataaggcactgcgagtcctgcaaaataacaacgtcgacctggtgaacataggcagcactgatattgttgatgggaaccataaactcacactgggtctgatttggaatattatactgcactggcaggtcaaaaatgttatgaaaaatattatggccgggcttcaacagaccaattccgagaagatcctgctgtcttgggttaggcagtccacgaggaactaccctcaagtgaacgttattaacttcacaacatcatggagtgatggacttgccctgaacgccttgatccactcccataggccagacctgttcgattggaatagcgtagtctgtcagcagagtgctacacaacgcctggaacacgcctttaacatagccaggtatcagcttggaattgagaaactcctcgaccctgaagacgtagacaccacttaccccgacaaaaaatccattcttatgtacataacttccttgtttcaggttctcccccagcaggtgtccatcgaggctatacaggaggtcgaaatgctccccagaccccctaaggtcacgaaggaagagcacttccaactgcaccatcagatgcactacagtcaacagatcactgtgagtctcgcacaggggtacgaaaggaccagctcccctaaaccccgcttcaagtcctacgcctatactcaggcagcttacgtaaccacaagtgaccccacgcggtcacccttcccaagccagcacctggaggctcccgaggacaaaagctttggatcatccctgatggaatctgaagtcaatctcgataggtaccaaaccgctctggaggaggtcctcagttggctcctttcagctgaagatactctgcaggcacagggtgagatcagcaatgacgtggaggtagtgaaagatcagtttcacactcacgagggctatatgatggacctgacagcccatcaaggtcgcgttggcaatatcctgcagttggggtccaaactgattggtaccggtaagttgtccgaggacgaagagacagaagtgcaggagcagatgaacctgcttaattctagatgggaatgcctccgcgtggcctctatggagaagcagtccaatctccatcgggttctcatggacttgcagaatcagaagctgaaggaactcaacgactggttgaccaagactgaggagcgcaccagaaagatggaggaggagccactcggtcctgatcttgaagatctgaaacgacaagttcagcagcacaaggtcctgcaggaggatctcgaacaggagcaggtccgggtgaacagcctgacccatatggttgtcgttgtcgacgagtcaagtggggaccatgctacggctgcgcttgaggaacagctgaaggtcttgggcgatagatgggcgaatatctgccgctggactgaagatagatgggtgctgctgcaggatattcttttgaagtggcagcggttgaccgaagaacaatgcttgtttagcgcctggctgagtgagaaagaggatgcggtgaataagatacacactactggcttcaaagaccagaatgaaatgcttagcagcctgcagaaacttgcggttcttaaggccgacttggaaaagaaaaagcagtcaatgggtaagctgtactcactcaagcaggacctgctgtcaactttgaagaacaagagcgtcacgcaaaagactgaggcctggctcgacaattttgctaggtgctgggataacctggtgcagaagttggaaaagtccactgctcagatctctcaagccgtgactactacccagcccagcctgacacagactaccgtcatggaaaccgttaccactgtgacgactagggagcagatactcgttaagcacgctcaagaggaattgccgccacctcctccccaaaagaaacgccagattaccgtcgactctgaaatacgcaagagactggatgtggacatcacggaactgcactcttggattaccagaagcgaggcagtcctccagagccccgagtttgcgatcttcaggaaggaaggcaacttttcagatctgaaggaaaaggtgaacgccatcgagcgcgagaaagccgagaagttccgcaaactgcaggatgcgagccggtctgctcaggccttggtggagcaaatggtgaatgaaggcgtaaatgcggacagcatcaagcaggcctccgagcagctcaattcaagatggatcgagttctgtcagctgctcagcgagcgattgaactggctggagtatcaaaataacataatcgcgttttacaaccagctgcaacagctggaacagatgacgactactgcagagaactggcttaaaatccagcctacaactccttctgaacccacagcaatcaaaagccagctcaagatctgcaaggacgaagtcaaccgcctctccgatttgcagcctcagatagaacgactcaagatccaaagtatcgcactcaaagaaaaggggcaaggccctatgtttctcgatgctgattttgtggctttcaccaaccattttaagcaagtgttttcagatgtgcaagcccgggaaaaggaactgcaaaccatttttgataccctgcctccaatgcggtaccaggagactatgtctgcaattcgcacctgggtgcaacaaagcgagacgaaactttctattccacaactgtccgtgacagactacgaaattatggaacagcgactgggagaactccaagcccttcagtccagccttcaggagcagcagtcaggattgtactatctgtcaaccaccgtgaaggaaatgtcaaaaaaggctccctccgaaatatctcgcaagtatcagtctgagtttgaagagattgaagggagatggaagaaactgtcctcacagctggttgagcactgtcagaagttggaagagcagatgaacaagttgcgcaagattcaaaatcacatacagacactcaaaaagtggatggcggaggttgacgtcttccttaaagaggagtggccagccctgggcgactcagagatcctgaaaaagcaactgaaacagtgccgactgctggtgtccgacatccagacaatacagccaagtttgaatagcgtgaatgagggtggacaaaaaatcaaaaacgaggctgagcccgagttcgcctccagattggaaaccgagctgaaggaacttaatacacagtgggatcatatgtgtcagcaggtttatgcccggaaggaagcactgaaaggggggctggagaaaaccgtgagccttcagaaggaccttagcgagatgcacgagtggatgacccaggcggaggaagagtatctcgaacgggattttgagtataagacaccagatgaacttcagaaggcagtcgaagagatgaaacgggcaaaggaagaggctcagcagaaggaagcaaaggttaaacttctgacagaatccgttaacagcgttatagcccaagctccccccgttgcccaggaagctcttaaaaaggaactggaaacactgacaacaaactaccagtggctgtgcacccgcttgaacggcaaatgcaaaaccctggaagaggtctgggcttgttggcatgaactcctgtcatacctggaaaaagctaataagtggctgaatgaggtggagtttaaattgaaaacgaccgaaaacatccctggcggtgcagaggagatatctgaggtgctcgatagcctggagaatcttatgaggcatagcgaggataaccccaaccagattaggatactggcccaaactctgaccgacggcggagtgatggacgagctgataaacgaggaactggaaacctttaattcacgctggcgagaacttcacgaggaggcagtcaggcgccagaaactgctggagcagtccatccagagtgcccaggagactgagaagtcacttcatctgatccaggagagtttgacgtttatcgacaagcagcttgccgcctacattgcagacaaggtcgatgctgctcagatgccgcaagaggctcagaaaatccagagcgacctgacaagccacgagattagcctggaggaaatgaaaaagcacaaccagggtaaagaggccgcccagcgggttctgagccagatcgatgtcgcacagaagaaactccaagatgtaagcatgaagttccgactgtttcagaaaccagcgaactttgagcagcgactgcaggagtctaaaatgatcctcgatgaggtgaaaatgcatctgccagccctggaaactaagagcgtggagcaggaggtggttcagagtcagctgaaccactgcgtaaacctctataaaagtcttagtgaagtgaagagcgaggtggagatggtgattaagactggtagacagattgtacagaaaaagcagacggagaaccccaaagagttggatgaacgggtgacagcccttaagctccactataacgagctgggagccaaagtgactgagagaaagcagcaacttgaaaaatgtctgaagctgagcagaaagatgcggaaggaaatgaatgtgctgacagagtggctggctgcgaccgacatggaacttacaaagaggagcgccgtggaaggcatgccttccaatctggatagtgaggtcgcttggggcaaagctacacagaaggaaattgagaagcaaaaagtgcatcttaagagcattactgaagttggcgaggctttgaaaaccgtcctcggcaagaaagagactctggtagaagataaactctcactgctgaactcaaactggatcgcagtcactagtcgggccgaagagtggctgaatttgctgttggagtaccaaaagcatatggagactttcgatcagaatgtcgaccacattacgaagtggataatccaggcggacacactgttggatgagtccgagaaaaagaaacctcagcagaaggaagacgtgctcaaacgactcaaagccgaactgaacgacatcaggcccaaagttgattccacccgggatcaagcggccaatctcatggccaaccggggggatcactgcagaaagttggtagaacctcagataagcgaacttaatcatagatttgccgccatatcacataggattaagaccggtaaggcttcaatccccctgaaggaactcgaacagtttaatagtgacatccagaagctcctggagcctctcgaagccgagattcaacagggtgtaaaccttaaggaagaggattttaataaggatatgaatgaggacaatgaaggcacggtcaaagagctcctccagaggggagacaatttgcagcagcggatcaccgatgaacgcaaacgcgaagagatcaaaattaagcagcagctcctccaaacaaagcacaatgcccttaaagacctcagaagccagcggcggaaaaaggctctggaaatcagccatcaatggtatcagtacaagcggcaagcggacgatctcctgaaatgtctggatgatatcgagaaaaaactggccagtctgccagagcccagagatgagcgcaaaatcaaggaaatcgacagggagcttcagaagaaaaaagaggagctgaacgccgtccgaaggcaggccgaagggctgtcagaagatggggccgccatggcagtggaacctacccagattcaactgtccaagcgctggagagagattgaaagtaagttcgcacaattccggaggcttaattttgctcagattcacacagttagggaggaaaccatgatggtgatgaccgaagacatgccactcgaaatcagctacgtccctagcacatatctcacggagattacccacgtgtcccaggcactgctggaagtcgagcaattgttgaacgcccctgatctctgcgccaaggatttcgaagacttgttcaagcaagaggaaagcctcaagaacatcaaagacagtcttcagcagtcctcaggcagaattgatattatccactccaagaaaacagccgctctccagagcgctacccctgtcgaacgagtgaaactgcaagaggccttgtctcagttggacttccagtgggagaaagtcaacaagatgtacaaggacaggcagggtcggttcgaccggagcgtcgaaaaatggcggagattccattatgacatcaagatattcaatcagtggcttacagaggcggagcaatttttgagaaagacgcagattcccgaaaactgggagcatgccaagtacaagtggtacctcaaagagctccaggatggaatcggccagagacagaccgtagttagaacactgaacgcaactggagaagagattatccagcagtcctccaaaactgacgcctctatcctccaggagaagcttggcagccttaacctgcggtggcaggaggtgtgtaaacaactgtcagataggaaaaaaagacttgaggaacagaagaacattctgagcgagttccaacgagatctcaacgagttcgtactgtggctggaggaggccgacaacatcgcttcaatccccctggaacccggcaaggaacagcagcttaaggaaaaattggaacaggtaaaactgttggtagaggagctcccattgcggcagggcattctcaagcagctgaatgaaactggaggtcccgttctggtcagtgcgcccataagcccggaagagcaggacaagttggaaaacaagctgaagcaaacaaatctgcagtggatcaaagtgagccgggctcttccagaaaagcaaggcgagattgaagcccagattaaggatcttggccagcttgagaaaaagctggaggatctggaagagcagctgaaccatttgcttctgtggctgagtcccattagaaaccagctggaaatctacaaccagccaaaccaagagggccctttcgacgtcaaggaaactgagatagcagtccaggcaaaacagcccgatgtcgaagaaatcttgtctaaggggcagcacctctacaaagagaaacctgccacccagcccgtgaagagaaaactggaagacctctcttcagagtggaaggctgtaaacaggttgctccaggagctgagagctaagcaacctgacctcgcacccggcctgactactattggcgccagcccgacccaaaccgtcacactggtgacccagcctgtagtcacaaaagaaaccgccatctccaaactggagatgccttcaagcctgatgctggaggtgcctgctctggccgatttcaacagagcctggacagagctgacagactggctgtctctgctcgaccaagtgataaagtcacagcgcgtcatggtgggtgacctggaggacatcaatgagatgatcataaagcaaaaggctaccatgcaggacttggagcagcgcaggccgcagttggaagaactgattacagcagcccagaacctcaagaacaagacttcaaatcaggaggcaaggaccatcataacagatagaatcgagaggattcagaaccaatgggatgaggtgcaggagcacctgcagaacagaaggcagcagctgaacgaaatgctgaaagactccacacagtggctggaggccaaagaggaggcagaacaggtgctgggccaggctagggccaagctggaaagttggaaggaagggccctacaccgtggacgccatacagaagaaaataacggagacaaaacagctggccaaagacctccgccaatggcagaccaacgtagacgtcgccaatgacctggcactcaagttgttgagagattattccgcagatgacacccgaaaagtacacatgatcactgagaatatcaacgccagttggcggagcattcacaaacgggtgagcgagagagaagccgcccttgaggagacgcatcgccttttgcagcaatttccgcttgacctggaaaagtttctggcttggttgacagaggccgagacaactgctaacgtgctgcaagatgccacccgcaaagagcggcttcttgaggactctaaaggggtgaaagaattgatgaagcagtggcaggacctccagggcgaaatcgaggcccatactgatgtctaccacaacctggatgaaaatagtcagaaaatcctgagatccctggaagggagtgatgatgcggtgctgctgcagcgccgactcgataacatgaactttaagtggtctgaactccggaaaaaaagcctgaacattcggagccatctggaggcttcttctgatcagtggaagagacttcatctgtccttgcaggagcttctggtgtggctgcagctgaaagacgacgagctgtcaaggcaggcaccaatagggggcgacttccccgctgttcagaaacaaaatgatgtgcacagggcattcaagcgggaactgaagacgaaagaaccagtgattatgagcactctggagacggtacgcatctttctgaccgagcagccacttgaaggcctcgaaaagttgtaccaggagccacgggaattgcctcccgaagaacgggctcagaatgttacccgactgcttcgcaagcaggctgaggaagtgaacactgagtgggaaaaactgaatctgcacagtgccgattggcagcgcaaaattgacgagacattggagcgcttgcgggagctgcaggaagccacagatgaacttgatctgaaactcagacaggccgaggtcataaagggttcttggcaacccgtaggcgaccttttgattgactcactgcaggatcatctggagaaagtaaaggcgctgcggggggaaattgctccgctgaaggaaaacgtgagccatgtgaatgatctggctcggcaattgactaccctgggtatccagctgtccccctacaatctgagcactctcgaagacctcaacacaagatggaaactgctgcaggtcgccgtggaggacagggtgaggcagctgcatgaggcacacagggacttcggtccggcaagccaacacttcctgtctacttctgttcaagggccttgggaacgcgctatttccccaaacaaagtgccgtactatattaaccatgaaactcagacaacctgctgggatcatcctaaaatgactgagctgtaccagagcctcgccgatctgaataacgtgaggtttagcgcatacagaaccgcaatgaagctcaggagactgcagaaggcactttgcctggacctcctgagcctctccgctgcctgcgatgccctcgaccagcacaacctgaagcagaatgatcagcctatggacatcctgcaaatcataaattgtctgacgacaatctacgaccggctcgaacaggagcacaataatctcgtgaacgttccactttgtgtggatatgtgtctcaactggctgttgaacgtctatgacaccgggagaactggaaggatccgcgttctgtcctttaagactggaatcatcagcttgtgtaaggcgcaccttgaggacaaatatcgatacctgtttaagcaggttgcaagctccactggattctgcgaccagaggcgcctcgggctgctgcttcatgatagtatccaaatcccacgccaactcggagaggtggcaagcttcggcgggagcaatattgagccaagcgtcagatcttgtttccagtttgccaacaataagcctgagatcgaagccgcactgtttttggattggatgcggttggagccccagagtatggtgtggttgcccgtgctgcatagggtcgctgccgctgaaaccgccaaacaccaggccaagtgcaatatttgcaaggaatgtcccatcatcgggtttagataccggagcctgaagcacttcaattatgatatctgccagtcttgttttttttccggccgagttgctaagggacacaagatgcattacccaatggtggaatattgtacgccaaccaccagcggcgaagatgtcagagattttgcaaaagtactcaagaacaagttccgcaccaagcgatatttcgcgaagcacccccggatgggctacctcccagtgcagacggttctggaaggcgataacatggaaactccagtcaccctgatcaatttctggcctgtggactctgcgcctgcctcttcacctcagctctcccacgatgacacccattccagaatcgagcactacgcgtctcgactggcggagatggagaacagcaatggttcttacctcaatgacagcattagccccaacgaaagcatcgatgacgagcacctcttgattcaacactactgccagtccctgaatcaggacagtcctctgtcccaacccaggagcccagcccagatactgatctctctcgaatcagaagaaagaggggagttggagcgaattttggccgacctggaggaggagaatagaaatcttcaggccgagtacgatagactgaagcagcagcacgagcacaagggcctttctccactgccctctccaccggaaatgatgccaacctcccctcaaagtcccagggacgctgagctcattgccgaggcaaagctgttgcgccagcacaaaggacgcctggaggcgagaatgcagatcctggaagaccataacaagcagcttgagtctcagctgcaccgacttagacagctgttggagcagccacaagcggaagcaaaggtcaatggaactaccgtgagttcccccagtacaagtctgcagcgctctgactcaagccagccaatgctgctccgggtagtcggttctcagactagtgattcaatgggggaggaggatcttctttccccaccccaagacacctcaacagggcttgaggaagtgatggagcagctgaacaactccttcccctcttcacgggggagaaatactcctgggaagcctatgagagaggataccatgtaatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 25 - DNA, 12889 bp, synthetic construct
  source 1..12889: mol_type = other DNA; note = MHCK7-hBGi-DMD-1-WPRE3-DTS
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaagaggtggaggattgctacgagcgggaggatgtgcagaaaaagactttcaccaagtgggtaaatgctcagttctctaagttcgggaagcaacacatcgagaacttgttttccgacctccaggatggcaggcgcctgcttgacttgctggagggtctgactggtcagaagctccctaaggagaagggcagcaccagagttcacgctctgaataacgttaacaaagccctgagggtcctccagaataacaatgtagacctggtgaacatcggttctaccgacatcgtggacgggaatcataaactgaccttggggctcatctggaacattatcttgcactggcaggtgaagaacgtcatgaagaatatcatggccgggctgcaacagaccaactccgaaaagattttgctcagctgggtgcgccagtccacccgcaattatccacaagtcaacgttatcaatttcaccactagttggagtgatggcctcgccctgaacgccttgatccacagtcaccgccccgacctgtttgactggaactctgtggtttgccaacagagcgctacccagcgtctcgaacatgctttcaatatcgctagataccagctcggtatcgagaagctgttggatcctgaggacgtagacacgacctaccccgataaaaagagcatcctgatgtatatcacttctctgtttcaggtgcttcctcaacaggtgtccattgaagccatccaagaggtcgaaatgctgcccaggccccctaaggtgacaaaagaagagcatttccagctgcatcaccagatgcattacagtcaacagattaccgtcagcttggctcagggatatgagcgcacgtcctctcctaagcccagatttaagtcctacgcatatacccaggctgcctacgtgaccacttccgacccaactaggagcccctttccctcccaacacctggaggcccctgaggacaaaagcttcggatctagtctgatggagtccgaggtgaatctggatcggtaccaaactgctctggaggaagtgctgtcctggcttctgtccgctgaagacaccctgcaggcgcaaggtgagatttctaacgacgtcgaggtcgtgaaggatcagttccacactcatgagggctacatgatggacttgacagctcaccagggaagagttggtaacatcctgcagcttggctccaagttgattggcacgggaaagcttagcgaggacgaggaaaccgaggtacaggagcagatgaacttgctgaattcccgctgggagtgcctgagagttgcgtccatggaaaagcagtcaaaccttcacagagtgctgatggaccttcagaaccagaagctgaaggagctgaacgattggctgacaaagaccgaggaacgcacaagaaaaatggaagaggaacctctgggacctgatctggaggatctgaagcgccaggtccaacagcataaggtgctgcaggaggatttggagcaggagcaggtgcgggtcaacagcctgacacatatggtggttgtggtcgatgagtcctctggcgatcatgctaccgcagcgctggaagagcagctgaaggtgctgggggatcgctgggccaacatttgcagatggacagaagaccggtgggtcctgcttcaagatatcctgctcaagtggcagcgcctgactgaagagcagtgcctgttctcagcttggttgagcgaaaaagaggacgcagttaacaagatccataccacaggcttcaaggatcagaatgagatgctgagctcactgcagaagctggctgtgctgaaggccgacttggagaagaaaaagcagtccatgggcaaactgtatagcctgaagcaggacctgctcagcaccctgaagaacaaaagcgttacgcagaaaactgaggcctggttggacaactttgctcgctgctgggataaccttgtgcagaagctggaaaagtcaaccgcacagatctcccaggctgtgaccactacgcagcctagcttgacccagacaaccgttatggaaaccgtgacaaccgtgacaacgagagagcaaatcctggttaagcatgcccaggaggaactgccgccacccccgcctcagaagaaacggcagattacggtggacagcgagatccgcaagaggctggacgtcgacatcactgagctccactcatggatcacgcggagtgaggccgtgctgcagtctcccgagttcgctattttccgcaaggagggaaacttctctgacttgaaagagaaggtgaatgccatcgagcgtgaaaaggcagaaaagttcagaaagctgcaggacgcctctcgctctgcgcaggccctggtggagcagatggtcaacgagggtgttaacgctgacagcatcaaacaggcaagcgaacagctgaactccagatggatcgaattttgccaactgctctccgaacgcctcaactggttggagtaccagaacaatattatcgctttttacaaccagctgcagcaactggagcagatgacaactaccgccgaaaactggctgaaaattcagccaaccacaccgtccgagccaactgcgatcaagtctcagctgaagatctgcaaggacgaagtgaaccgtcttagcgacttgcaaccccagattgaacggctcaagattcagtctattgctctgaaggagaagggtcagggtccgatgttcctggatgcagattttgtcgcatttaccaatcacttcaaacaggtgtttagtgacgtccaggcgcgcgagaaagagctgcagactatttttgacaccctgcctcccatgaggtaccaggagacaatgagtgccattcgcacctgggtgcaacagtccgagacgaaattgtcaatcccacagctgtccgtgacagactacgagattatggagcaacggttgggggagctgcaggctcttcagagttccctgcaggagcaacagagcggactgtactatctttccactaccgtgaaggaaatgagtaagaaagccccttccgagatcagccgcaaataccagtctgaattcgaagagatcgagggacgctggaaaaagctgtcaagccagctggtcgagcactgtcagaagctggaagagcagatgaacaagctgcgtaaaatccagaaccatattcagaccctcaagaaatggatggcagaagttgacgtgtttctgaaggaagagtggcctgccctcggcgatagcgaaatccttaagaaacaactcaagcagtgccgcctcctggtgtccgatattcagacaatccagccttccctgaacagcgttaatgagggcggacagaaaattaaaaacgaggccgaacccgagttcgcttctcgcctggagactgaactgaaggagctgaatacccagtgggaccatatgtgtcaacaggtctacgcacgcaaagaggcactgaagggcggtctggagaagactgtctcactccagaaggacctgagcgagatgcacgagtggatgacccaggccgaagaggaatacctggagcgtgattttgagtataagaccccagacgaactccagaaagccgttgaagagatgaagagggctaaggaggaagcccaacagaaggaggcgaaagtgaagctcctgacagagtccgtgaactccgtcatcgctcaagctcctcccgtggctcaggaggccctcaagaaagagctggaaactctgaccacaaattaccagtggttgtgtacccgcttgaacggtaagtgcaagaccctggaagaggtgtgggcctgctggcacgagctcctgtcctatcttgagaaggcgaacaaatggctgaacgaagtggaattcaagttgaaaacaactgagaacattccgggaggcgctgaggaaatctctgaggtgctggactctctggaaaatctgatgcgtcattctgaagacaatcctaaccagatcaggattctggcacagaccctcactgacggaggcgttatggacgaacttatcaacgaggaactggagacattcaattcccgttggcgtgagctgcacgaagaggccgtgcgccgtcagaagttgctggaacagtctatccagtccgcccaggagacagagaaatctctccacctgatccaggagagtctgacgtttatcgataagcagctggctgcctacatcgccgacaaggtggacgctgcccagatgccccaagaggctcagaagatccagtccgacctgacgtctcatgaaatcagcctggaagagatgaaaaagcacaaccagggcaaagaggctgcccagcgcgtcctctcccagattgacgtggctcagaaaaagttgcaggacgtgagcatgaagtttaggctgttccagaaacccgccaacttcgaacagagactgcaggagtctaagatgatcctggatgaggtcaagatgcacctccctgctctggaaacaaaatctgtggagcaggaggtcgtgcagtcacaactgaaccattgtgtgaacctctacaagtctctcagcgaggtgaagtccgaggtcgagatggtgatcaagaccggacgccagattgtgcagaaaaagcagacagagaacccaaaggagctggacgaacgggtgaccgccctgaagctccactacaacgagttgggtgccaaagtcactgaaagaaagcaacagctggagaagtgtctgaagctgagccgcaagatgcgcaaagaaatgaacgtcttgaccgagtggttggcggccaccgacatggagctgaccaagagatccgctgtggagggcatgccctccaaccttgactcagaggtggcctggggcaaggccacacagaaggagatcgaaaaacagaaggtgcatcttaaatcaatcactgaggtgggcgaagctctgaagacagtgctgggcaagaaagagacacttgtagaggacaagctctcccttctgaatagcaattggatcgccgtaactagtcgcgcggaggaatggctgaacctcctgttggaataccagaaacacatggaaaccttcgatcagaacgtggaccatattaccaagtggattatccaggctgacactctcctggacgagtctgagaaaaagaaaccccagcaaaaggaagatgtgctgaagcgcctgaaggcggaattgaacgacatccgcccgaaggttgattccacccgcgaccaagcggccaaccttatggctaaccgcggggaccattgcagaaaactggtggaaccgcagatctctgagctgaaccacagattcgccgcgatttctcacagaattaagactggcaaggcctccatccctctgaaagagctggagcagttcaactctgatattcagaagcttctggagcccctggaggcagaaatccaacagggcgtcaacctgaaagaggaagactttaataaggacatgaatgaggataacgaaggtaccgtcaaggagctcctgcagaggggcgacaatctgcagcaaagaatcactgacgaacgtaagcgcgaagagattaagatcaagcaacagctgttgcagactaagcacaacgccctgaaggatctgaggtcccagcgcaggaaaaaggccctggagatttcccaccagtggtaccagtacaagcgccaggccgatgaccttctgaagtgcctcgacgatatcgagaaaaagctggcctcactgcccgagccccgcgacgagcgcaagatcaaggagattgaccgtgagctccagaagaaaaaggaggaactgaacgcggtgcgcagacaggccgagggcctctccgaggacggcgctgcgatggctgttgagcccactcagatccaactgagcaagcggtggagagagatcgagtctaagttcgcgcaatttaggcgcctcaacttcgcccagattcacacggtccgtgaggaaacgatgatggtgatgacagaggatatgccactggaaatcagctacgtgcccagtacatatctcaccgaaatcacccatgtgtcccaggctctcctggaggtggaacagttgctcaacgctcccgacttgtgcgctaaggacttcgaggacctgttcaagcaggaagagagcctgaaaaacattaaggactccctgcaacagtcaagcggccgcatcgatatcattcattctaagaaaaccgctgccctgcagtctgcaacccccgtagagcgcgtcaaactgcaggaagctctcagccaactggatttccagtgggagaaggtgaacaagatgtataaggatcgtcagggccggttcgaccgctctgtggagaagtggcgccgtttccactacgatatcaagattttcaatcaatggttgaccgaggcagagcagttcctcagaaagacacagatccctgaaaattgggagcatgccaagtacaagtggtatctgaaggagctgcaggacgggatcggacaacgtcagaccgtggtccgcaccctgaacgcaaccggcgaggaaattatccaacagtctagcaagacagatgctagcatcctccaggagaagctcggaagcctcaaccttcgctggcaggaggtgtgcaagcagctgtcagacaggaagaaacgcctggaggaacagaagaacatcctttcagagttccagagagatctgaacgaattcgtgctttggcttgaagaggccgataacatcgcaagcatcccactggagcctggtaaggaacagcaattgaaggagaagctggagcaggtaaagctgctcgtggaagagctgcctctgaggcagggaattctcaagcagctcaacgagactgggggcccggttttggtgagcgccccaattagcccagaagagcaggacaagttggagaacaagctgaagcagaccaacctgcagtggatcaaggtgtcccgcgcactgcccgaaaagcagggagagattgaggcccagatcaaggatctgggacagctggagaaaaagctggaagacctggaggaacagctgaaccatttgctcctgtggttgtcccccatcagaaatcagctggaaatctacaaccaaccgaaccaggaaggtccgtttgatgttaaggaaaccgagatcgctgtgcaggcgaagcagcctgacgtagaagagatcctgtcaaagggacagcacctctacaaggagaagcccgccacccagccagtcaagcggaagctcgaagacctgtccagcgagtggaaagccgtcaaccgcctcctgcaagagctgcgtgccaagcaaccggacctggcgccaggtctgactaccattggcgcaagtcctacccagactgtcaccctggtgacccaacccgttgtgacaaaggaaaccgccatcagcaagctggagatgcctagctctttgatgctggaggttcccgccctcgccgacttcaaccgtgcttggacagaactgaccgactggctgtccctcctggaccaagtgatcaagagccagcgcgtgatggtgggtgatcttgaggatattaacgaaatgattatcaagcagaaggccaccatgcaggacttggagcagaggcgcccccagttggaggaactgatcaccgccgcgcagaatctgaaaaacaaaacttccaatcaggaagcgagaaccatcattacagacagaattgagagaattcaaaaccagtgggacgaagtgcaagaacacctccagaaccggcgtcaacagctgaacgagatgctgaaggattccactcaatggctggaagctaaggaagaggccgagcaggtccttggacaggcacgcgctaagctggagtcctggaaagaaggcccctatacagtggacgcaattcaaaagaaaatcaccgagacgaagcaactggccaaggacctgagacagtggcaaaccaacgttgatgtggctaatgacctggccttgaagctcctgcgtgactactccgcggatgacactagaaaggtgcacatgatcaccgagaatatcaacgctagctggcgctccatccataagcgtgtgagtgagcgcgaagccgctctggaggaaacccatcgcctgcttcaacagtttcctttggacctggaaaagttcctggcctggctcacagaagccgagacaaccgccaacgtgctgcaggacgccacccgtaaggagcgcttgctggaggatagtaagggggtgaaggagttgatgaagcagtggcaagatctgcagggggaaatcgaggcacataccgacgtctatcataacctggatgagaattcccagaaaatcctgcgcagcttggagggcagtgatgacgcggtgcttctgcaacggcgcttggacaacatgaacttcaagtggtccgagttgcgtaaaaagagtctgaacattcgctcccacctggaggcttctagtgaccagtggaagcgcctgcacctgagcctgcaagaactgctcgtgtggctgcaactgaaggatgacgagttgagtcgtcaggcccctatcggaggtgactttccggctgtccagaaacagaacgacgtgcatagggcatttaagcgcgagttgaagacaaaggagccagtcattatgtccacgctggaaaccgtgcgcattttcctgactgagcagcctctggaggggctggagaagctgtaccaggagcctcgcgagttgccccctgaagagcgtgcccagaatgtgacccgcctgttgcgtaagcaggccgaggaagtgaacaccgagtgggagaagctgaacctgcattccgcggattggcagaggaagattgacgagacgctggagaggctccgtgagttgcaggaggcaaccgacgaactggacctcaagctgcgtcaagcagaggtgatcaaggggagttggcagccggtcggcgatttgctgatcgactccctgcaggatcacctggaaaaggttaaagctttgcgcggggaaattgccccgctgaaggagaacgtgagccacgttaacgatctggccagacagctgaccactctgggtatccagctgagcccgtacaatctgagcaccctcgaagacttgaatacgagatggaaactcctgcaggtggcagtggaggacagggtgcgccaattgcacgaggcccaccgggactttggaccggccagccagcatttcctcagtacctctgttcagggcccgtgggagcgggccatctcccccaacaaggtcccctattacatcaaccatgaaacccagactacctgttgggaccaccctaaaatgactgagttgtatcaatctctggccgacttgaataacgtgcgcttctcagcctacaggactgcaatgaaactcagacgcctccaaaaggccctttgtctggacttgctgtcccttagcgctgcctgtgatgctctggatcagcacaatttgaaacaaaacgaccagccgatggacatcctccagattatcaactgcctgactaccatctacgacagactggaacaggagcataataacctggtgaacgtccccctgtgcgtggacatgtgcctgaactggctgctcaatgtgtatgacacgggtagaacaggacggatcagggtcctgtcctttaagaccggtatcatttctctgtgtaaggcacatctggaggacaagtaccgctacctgtttaagcaggtcgccagctccactggcttctgcgatcaaaggcggctgggtctgctcctgcacgactccattcagatcccgagacagttgggtgaagtggcgtccttcggcggttctaatatcgagcctagcgtccgcagctgtttccaattcgccaataacaagccggagatcgaagcggcccttttcctggactggatgcgtctggaacctcagagcatggtttggcttccggtgcttcatcgcgtggccgctgccgaaaccgctaaacatcaggccaaatgtaacatctgtaaggaatgcccgattatcggtttccggtaccgtagcctcaagcatttcaactatgatatttgtcaatcttgcttctttagcggacgggtggccaaaggccacaaaatgcactacccgatggtcgagtattgcactcctacaacctccggcgaggacgtgcgcgattttgccaaagtgctgaaaaacaagtttagaaccaagcgttactttgccaagcatccacgtatgggctacctgcctgtccagaccgttctggaaggtgataacatggagacacccgtgactttgattaacttttggccagtagactctgctccggcaagctcccctcagctctcccacgatgacactcacagccgtatcgagcactacgcctctcgcctggctgagatggagaactctaacggttcatacctgaatgacagtatctccccaaacgagagtatcgatgacgaacatctgctcattcagcattactgtcaatccttgaatcaggacagccccctgtcccagccacggtccccggcccagatcctgatttcactggagtccgaagagagaggggaactggaaaggatcctcgctgacctggaagaggaaaaccggaacctgcaggctgaatacgaccgcttgaagcagcaacacgagcacaagggcttgagcccgttgccaagccctccggagatgatgccaactagccctcagagcccacgggatgccgagctcatcgctgaagctaagttgctgagacagcacaagggacgtctggaggcccgcatgcagatcctggaggatcataataaacagttggagagccaactgcataggctgcgccagctcctggagcagccacaggccgaggctaaggtcaacggaactaccgtttccagcccgtccacatctctgcagcgctcagatagttcccagcctatgttgctgagagtggttggatctcagacctccgactcaatgggcgaagaggacttgctgtctcccccacaggacactagcacaggactcgaagaggtgatggaacagctcaataactcttttccttctagtcggggtcgcaatacccctggaaagcccatgcgcgaagacaccatgtaatagctcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 26 - DNA, 12889 bp, synthetic construct; identical to SEQ ID NO: 6
  source 1..12889: mol_type = other DNA; note = MHCK7-hBGi-DMD-3-WPRE3-DTS

SEQ ID NO: 27 - DNA, 12889 bp, synthetic construct; identical to SEQ ID NO: 7
  source 1..12889: mol_type = other DNA; note = MHCK7-hBGi-DMD-4-WPRE3-DTS

SEQ ID NO: 28 - DNA, 12889 bp, synthetic construct
  source 1..12889: mol_type = other DNA; note = MHCK7-hBGi-DMD-5-WPRE3-DTS
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaggaagttgaagattgttatgagagagaagatgtccagaaaaagacctttaccaaatgggtgaacgcacagttctccaaatttgggaaacagcatatagaaaatttgttcagcgatcttcaggatgggcgcaggttgcttgaccttctggaaggactgaccgggcagaagctgcccaaagagaaagggtctaccagagttcacgctctgaacaacgtgaataaggcactgcgagtcctgcaaaataacaacgtcgacctggtgaacataggcagcactgatattgttgatgggaaccataaactcacactgggtctgatttggaatattatactgcactggcaggtcaaaaatgttatgaaaaatattatggccgggcttcaacagaccaattccgagaagatcctgctgtcttgggttaggcagtccacgaggaactaccctcaagtgaacgttattaacttcacaacatcatggagtgatggacttgccctgaacgccttgatccactcccataggccagacctgttcgattggaatagcgtagtctgtcagcagagtgctacacaacgcctggaacacgcctttaacatagccaggtatcagcttggaattgagaaactcctcgaccctgaagacgtagacaccacttaccccgacaaaaaatccattcttatgtacataacttccttgtttcaggttctcccccagcaggtgtccatcgaggctatacaggaggtcgaaatgctccccagaccccctaaggtcacgaaggaagagcacttccaactgcaccatcagatgcactacagtcaacagatcactgtgagtctcgcacaggggtacgaaaggaccagctcccctaaaccccgcttcaagtcctacgcctatactcaggcagcttacgtaaccacaagtgaccccacgcggtcacccttcccaagccagcacctggaggctcccgaggacaaaagctttggatcatccctgatggaatctgaagtcaatctcgataggtaccaaaccgctctggaggaggtcctcagttggctcctttcagctgaagatactctgcaggcacagggtgagatcagcaatgacgtggaggtagtgaaagatcagtttcacactcacgagggctatatgatggacctgacagcccatcaaggtcgcgttggcaatatcctgcagttggggtccaaactgattggtaccggtaagttgtccgaggacgaagagacagaagtgcaggagcagatgaacctgcttaattctagatgggaatgcctccgcgtggcctctatggagaagcagtccaatctccatcgggttctcatggacttgcagaatcagaagctgaaggaactcaacgactggttgaccaagactgaggagcgcaccagaaagatggaggaggagccactcggtcctgatcttgaagatctgaaacgacaagttcagcagcacaaggtcctgcaggaggatctcgaacaggagcaggtccgggtgaacagcctgacccatatggttgtcgttgtcgacgagtcaagtggggaccatgctacggctgcgcttgaggaacagctgaaggtcttgggcgatagatgggcgaatatctgccgctggactgaagatagatgggtgctgctgcaggatattcttttgaagtggcagcggttgaccgaagaacaatgcttgtttagcgcctggctgagtgagaaagaggatgcggtgaataagatacacactactggcttcaaagaccagaatgaaatgcttagcagcctgcagaaacttgcggttcttaaggccgacttggaaaagaaaaagcagtcaatgggtaagctgtactcactcaagcaggacctgctgtcaactttgaagaacaagagcgtcacgcaaaagactgaggcctggctcgacaattttgctaggtgctgggataacctggtgcagaagttggaaaagtccactgctcagatctctcaagccgtgactactacccagcccagcctgacacagactaccgtcatggaaaccgttaccactgtgacgactagggagcagatactcgttaagcacgctcaagaggaattgccgccacctcctccccaaaagaaacgccagattaccgtcgactctgaaatacgcaagagactggatgtggacatcacggaactgcactcttggattaccagaagcgaggcagtcctccagagccccgagtttgcgatcttcaggaaggaaggcaacttttcagatctgaaggaaaaggtgaacgccatcgagcgcgagaaagccgagaagttccgcaaactgcaggatgcgagccggtctgctcaggccttggtggagcaaatggtgaatgaaggcgtaaatgcggacagcatcaagcaggcctccgagcagctcaattcaagatggatcgagttctgtcagctgctcagcgagcgattgaactggctggagtatcaaaataacataatcgcgttttacaaccagctgcaacagctggaacagatgacgactactgcagagaactggcttaaaatccagcctacaactccttctgaacccacagcaatcaaaagccagctcaagatctgcaaggacgaagtcaaccgcctctccgatttgcagcctcagatagaacgactcaagatccaaagtatcgcactcaaagaaaaggggcaaggccctatgtttctcgatgctgattttgtggctttcaccaaccattttaagcaagtgttttcagatgtgcaagcccgggaaaaggaactgcaaaccatttttgataccctgcctccaatgcggtaccaggagactatgtctgcaattcgcacctgggtgcaacaaagcgagacgaaactttctattccacaactgtccgtgacagactacgaaattatggaacagcgactgggagaactccaagcccttcagtccagccttcaggagcagcagtcaggattgtactatctgtcaaccaccgtgaaggaaatgtcaaaaaaggctccctccgaaatatctcgcaagtatcagtctgagtttgaagagattgaagggagatggaagaaactgtcctcacagctggttgagcactgtcagaagttggaagagcagatgaacaagttgcgcaagattcaaaatcacatacagacactcaaaaagtggatggcggaggttgacgtcttccttaaagaggagtggccagccctgggcgactcagagatcctgaaaaagcaactgaaacagtgccgactgctggtgtccgacatccagacaatacagccaagtttgaatagcgtgaatgagggtggacaaaaaatcaaaaacgaggctgagcccgagttcgcctccagattggaaaccgagctgaaggaacttaatacacagtgggatcatatgtgtcagcaggtttatgcccggaaggaagcactgaaaggggggctggagaaaaccgtgagccttcagaaggaccttagcgagatgcacgagtggatgacccaggcggaggaagagtatctcgaacgggattttgagtataagacaccagatgaacttcagaaggcagtcgaagagatgaaacgggcaaaggaagaggctcagcagaaggaagcaaaggttaaacttctgacagaatccgttaacagcgttatagcccaagctccccccgttgcccaggaagctcttaaaaaggaactggaaacactgacaacaaactaccagtggctgtgcacccgcttgaacggcaaatgcaaaaccctggaagaggtctgggcttgttggcatgaactcctgtcatacctggaaaaagctaataagtggctgaatgaggtggagtttaaattgaaaacgaccgaaaacatccctggcggtgcagaggagatatctgaggtgctcgatagcctggagaatcttatgaggcatagcgaggataaccccaaccagattaggatactggcccaaactctgaccgacggcggagtgatggacgagctgataaacgaggaactggaaacctttaattcacgctggcgagaacttcacgaggaggcagtcaggcgccagaaactgctggagcagtccatccagagtgcccaggagactgagaagtcacttcatctgatccaggagagtttgacgtttatcgacaagcagcttgccgcctacattgcagacaaggtcgatgctgctcagatgccgcaagaggctcagaaaatccagagcgacctgacaagccacgagattagcctggaggaaatgaaaaagcacaaccagggtaaagaggccgcccagcgggttctgagccagatcgatgtcgcacagaagaaactccaagatgtaagcatgaagttccgactgtttcagaaaccagcgaactttgagcagcgactgcaggagtctaaaatgatcctcgatgaggtgaaaatgcatctgccagccctggaaactaagagcgtggagcaggaggtggttcagagtcagctgaaccactgcgtaaacctctataaaagtcttagtgaagtgaagagcgaggtggagatggtgattaagactggtagacagattgtacagaaaaagcagacggagaaccccaaagagttggatgaacgggtgacagcccttaagctccactataacgagctgggagccaaagtgactgagagaaagcagcaacttgaaaaatgtctgaagctgagcagaaagatgcggaaggaaatgaatgtgctgacagagtggctggctgcgaccgacatggaacttacaaagaggagcgccgtggaaggcatgccttccaatctggatagtgaggtcgcttggggcaaagctacacagaaggaaattgagaagcaaaaagtgcatcttaagagcattactgaagttggcgaggctttgaaaaccgtcctcggcaagaaagagactctggtagaagataaactctcactgctgaactcaaactggatcgcagtcactagtcgggccgaagagtggctgaatttgctgttggagtaccaaaagcatatggagactttcgatcagaatgtcgaccacattacgaagtggataatccaggcggacacactgttggatgagtccgagaaaaagaaacctcagcagaaggaagacgtgctcaaacgactcaaagccgaactgaacgacatcaggcccaaagttgattccacccgggatcaagcggccaatctcatggccaaccggggggatcactgcagaaagttggtagaacctcagataagcgaacttaatcatagatttgccgccatatcacataggattaagaccggtaaggcttcaatccccctgaaggaactcgaacagtttaatagtgacatccagaagctcctggagcctctcgaagccgagattcaacagggtgtaaaccttaaggaagaggattttaataaggatatgaatgaggacaatgaaggcacggtcaaagagctcctccagaggggagacaatttgcagcagcggatcaccgatgaacgcaaacgcgaagagatcaaaattaagcagcagctcctccaaacaaagcacaatgcccttaaagacctcagaagccagcggcggaaaaaggctctggaaatcagccatcaatggtatcagtacaagcggcaagcggacgatctcctgaaatgtctggatgatatcgagaaaaaactggccagtctgccagagcccagagatgagcgcaaaatcaaggaaatcgacagggagcttcagaagaaaaaagaggagctgaacgccgtccgaaggcaggccgaagggctgtcagaagatggggccgccatggcagtggaacctacccagattcaactgtccaagcgctggagagagattgaaagtaagttcgcacaattccggaggcttaattttgctcagattcacacagttagggaggaaaccatgatggtgatgaccgaagacatgccactcgaaatcagctacgtccctagcacatatctcacggagattacccacgtgtcccaggcactgctggaagtcgagcaattgttgaacgcccctgatctctgcgccaaggatttcgaagacttgttcaagcaagaggaaagcctcaagaacatcaaagacagtcttcagcagtcctcaggcagaattgatattatccactccaagaaaacagccgctctccagagcgctacccctgtcgaacgagtgaaactgcaagaggccttgtctcagttggacttccagtgggagaaagtcaacaagatgtacaaggacaggcagggtcggttcgaccggagcgtcgaaaaatggcggagattccattatgacatcaagatattcaatcagtggcttacagaggcggagcaatttttgagaaagacgcagattcccgaaaactgggagcatgccaagtacaagtggtacctcaaagagctccaggatggaatcggccagagacagaccgtagttagaacactgaacgcaactggagaagagattatccagcagtcctccaaaactgacgcctctatcctccaggagaagcttggcagccttaacctgcggtggcaggaggtgtgtaaacaactgtcagataggaaaaaaagacttgaggaacagaagaacattctgagcgagttccaacgagatctcaacgagttcgtactgtggctggaggaggccgacaacatcgcttcaatccccctggaacccggcaaggaacagcagcttaaggaaaaattggaacaggtaaaactgttggtagaggagctcccattgcggcagggcattctcaagcagctgaatgaaactggaggtcccgttctggtcagtgcgcccataagcccggaagagcaggacaagttggaaaacaagctgaagcaaacaaatctgcagtggatcaaagtgagccgggctcttccagaaaagcaaggcgagattgaagcccagattaaggatcttggccagcttgagaaaaagctggaggatctggaagagcagctgaaccatttgcttctgtggctgagtcccattagaaaccagctggaaatctacaaccagccaaaccaagagggccctttcgacgtcaaggaaactgagatagcagtccaggcaaaacagcccgatgtcgaagaaatcttgtctaaggggcagcacctctacaaagagaaacctgccacccagcccgtgaagagaaaactggaagacctctcttcagagtggaaggctgtaaacaggttgctccaggagctgagagctaagcaacctgacctcgcacccggcctgactactattggcgccagcccgacccaaaccgtcacactggtgacccagcctgtagtcacaaaagaaaccgccatctccaaactggagatgccttcaagcctgatgctggaggtgcctgctctggccgatttcaacagagcctggacagagctgacagactggctgtctctgctcgaccaagtgataaagtcacagcgcgtcatggtgggtgacctggaggacatcaatgagatgatcataaagcaaaaggctaccatgcaggacttggagcagcgcaggccgcagttggaagaactgattacagcagcccagaacctcaagaacaagacttcaaatcaggaggcaaggaccatcataacagatagaatcgagaggattcagaaccaatgggatgaggtgcaggagcacctgcagaacagaaggcagcagctgaacgaaatgctgaaagactccacacagtggctggaggccaaagaggaggcagaacaggtgctgggccaggctagggccaagctggaaagttggaaggaagggccctacaccgtggacgccatacagaagaaaataacggagacaaaacagctggccaaagacctccgccaatggcagaccaacgtagacgtcgccaatgacctggcactcaagttgttgagagattattccgcagatgacacccgaaaagtacacatgatcactgagaatatcaacgccagttggcggagcattcacaaacgggtgagcgagagagaagccgcccttgaggagacgcatcgccttttgcagcaatttccgcttgacctggaaaagtttctggcttggttgacagaggccgagacaactgctaacgtgctgcaagatgccacccgcaaagagcggcttcttgaggactctaaaggggtgaaagaattgatgaagcagtggcaggacctccagggcgaaatcgaggcccatactgatgtctaccacaacctggatgaaaatagtcagaaaatcctgagatccctggaagggagtgatgatgcggtgctgctgcagcgccgactcgataacatgaactttaagtggtctgaactccggaaaaaaagcctgaacattcggagccatctggaggcttcttctgatcagtggaagagacttcatctgtccttgcaggagcttctggtgtggctgcagctgaaagacgacgagctgtcaaggcaggcaccaatagggggcgacttccccgctgttcagaaacaaaatgatgtgcacagggcattcaagcgggaactgaagacgaaagaaccagtgattatgagcactctggagacggtacgcatctttctgaccgagcagccacttgaaggcctcgaaaagttgtaccaggagccacgggaattgcctcccgaagaacgggctcagaatgttacccgactgcttcgcaagcaggctgaggaagtgaacactgagtgggaaaaactgaatctgcacagtgccgattggcagcgcaaaattgacgagacattggagcgcttgcgggagctgcaggaagccacagatgaacttgatctgaaactcagacaggccgaggtcataaagggttcttggcaacccgtaggcgaccttttgattgactcactgcaggatcatctggagaaagtaaaggcgctgcggggggaaattgctccgctgaaggaaaacgtgagccatgtgaatgatctggctcggcaattgactaccctgggtatccagctgtccccctacaatctgagcactctcgaagacctcaacacaagatggaaactgctgcaggtcgccgtggaggacagggtgaggcagctgcatgaggcacacagggacttcggtccggcaagccaacacttcctgtctacttctgttcaagggccttgggaacgcgctatttccccaaacaaagtgccgtactatattaaccatgaaactcagacaacctgctgggatcatcctaaaatgactgagctgtaccagagcctcgccgatctgaataacgtgaggtttagcgcatacagaaccgcaatgaagctcaggagactgcagaaggcactttgcctggacctcctgagcctctccgctgcctgcgatgccctcgaccagcacaacctgaagcagaatgatcagcctatggacatcctgcaaatcataaattgtctgacgacaatctacgaccggctcgaacaggagcacaataatctcgtgaacgttccactttgtgtggatatgtgtctcaactggctgttgaacgtctatgacaccgggagaactggaaggatccgcgttctgtcctttaagactggaatcatcagcttgtgtaaggcgcaccttgaggacaaatatcgatacctgtttaagcaggttgcaagctccactggattctgcgaccagaggcgcctcgggctgctgcttcatgatagtatccaaatcccacgccaactcggagaggtggcaagcttcggcgggagcaatattgagccaagcgtcagatcttgtttccagtttgccaacaataagcctgagatcgaagccgcactgtttttggattggatgcggttggagccccagagtatggtgtggttgcccgtgctgcatagggtcgctgccgctgaaaccgccaaacaccaggccaagtgcaatatttgcaaggaatgtcccatcatcgggtttagataccggagcctgaagcacttcaattatgatatctgccagtcttgttttttttccggccgagttgctaagggacacaagatgcattacccaatggtggaatattgtacgccaaccaccagcggcgaagatgtcagagattttgcaaaagtactcaagaacaagttccgcaccaagcgatatttcgcgaagcacccccggatgggctacctcccagtgcagacggttctggaaggcgataacatggaaactccagtcaccctgatcaatttctggcctgtggactctgcgcctgcctcttcacctcagctctcccacgatgacacccattccagaatcgagcactacgcgtctcgactggcggagatggagaacagcaatggttcttacctcaatgacagcattagccccaacgaaagcatcgatgacgagcacctcttgattcaacactactgccagtccctgaatcaggacagtcctctgtcccaacccaggagcccagcccagatactgatctctctcgaatcagaagaaagaggggagttggagcgaattttggccgacctggaggaggagaatagaaatcttcaggccgagtacgatagactgaagcagcagcacgagcacaagggcctttctccactgccctctccaccggaaatgatgccaacctcccctcaaagtcccagggacgctgagctcattgccgaggcaaagctgttgcgccagcacaaaggacgcctggaggcgagaatgcagatcctggaagaccataacaagcagcttgagtctcagctgcaccgacttagacagctgttggagcagccacaagcggaagcaaaggtcaatggaactaccgtgagttcccccagtacaagtctgcagcgctctgactcaagccagccaatgctgctccgggtagtcggttctcagactagtgattcaatgggggaggaggatcttctttccccaccccaagacacctcaacagggcttgaggaagtgatggagcagctgaacaactccttcccctcttcacgggggagaaatactcctgggaagcctatgagagaggataccatgtaatgactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttct

SEQ ID NO: 29 - DNA, 4594 bp, synthetic construct
  source 1..4594: mol_type = other DNA; note = NP-CBA-hBGi-EGFP-P2A-Fluc-WPRE3-DTS
tgcatgcggccgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaatcgcgctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 30 - DNA, 4786 bp, synthetic construct
  source 1..4786: mol_type = other DNA; note = NP-MHCK7-hBGi-EGFP-P2A-Fluc-WPRE3-DTS
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaatcgcgctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggcccgtcgacttaagtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattagcggccgc

SEQ ID NO: 31 - DNA, 15459 bp, synthetic construct
  source 1..15459: mol_type = other DNA; note = pUC57-CBA-hBGi-DMD-4-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatgcggccgccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtc

SEQ ID NO: 32 - DNA, 15634 bp, synthetic construct
  source 1..15634: mol_type = other DNA; note = pUC57-MHCK7-hBGi-DMD-4-WPRE3-DTS
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 33 - DNA, 13428 bp, synthetic construct
  source 1..13428: mol_type = other DNA; note = NP-MHCK7-hBGi-mafaDMD
agctcggtacctcgcgaatgcatctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctgtggtgggaggaggtggaggattgctacgagagggaagatgtccagaagaaaaccttcaccaagtgggtgaacgcccagtttagcaagttcggcaagcagcacatcgagaacctgtttagcgacctgcaggacggcagaaggctgctggacctcctggaggggctgaccggccagaagctgcccaaggaaaagggcagcaccagggtgcacgccctgaataacgtgaacaaggccctgagagtgctgcagaacaacaacgtggacctggtgaacatcggcagcaccgacatcgtggacggcaaccacaagctgaccctgggcctgatctggaatatcatcctgcactggcaggtgaagaacgtgatgaagaacatcatggccggcctgcagcagaccaacagcgagaagatcctgctgagctgggtgagacagagcaccaggaactacccccaggtgaacgtgatcaacttcaccaccagctggagcgacgggctggccctgaacgccctgatccacagccacaggcccgacctgttcgactggaacagcgtggtgtgccaacagagcgccacgcagaggctggagcacgccttcaacatcgccagataccagctgggcatcgagaagctgttggaccccgaggacgtggccaccacctaccccgacaagaagagcatcctgatgtacatcaccagcctgttccaggtgctgccccagcaggtgagcatcgaggccatccaggaggtggagatgctgcccaggccccccaaggtgacaaaggaggagcacttccagctgcaccaccagatgcactacagccagcagatcacggtgtcactggcccagggctacgagaggaccagcagccccaagcccagattcaagagctacgcctacacccaggccgcctacgtgaccaccagcgaccccaccaggtcccccttccccagccagcacctggaggcccccgaggataagagtttcggcagcagcctcatggagagcgaagtgaacctggacagataccagaccgccctcgaagaggtgctgagctggctgctgagcgccgaggataccctgcaggcccagggcgagatcagcaacgacgtggaggtggtgaaggagcagttccacgcccacgagggctacatgatggacctgaccgcccaccagggcagggtgggcaacatcctccaactgggcagccagctgatcggcaccggcaagctgagcgaggacgaggagactgaggtgcaggagcagatgaacctgctgaacagcaggtgggagtgcctgagggtggccagcatggagaaacagtccaacctgcacagggtcctgatggacctgcagaaccagaagctcaaggaactcaacgactggctgaccaagaccgaggagagaaccaggaagatggaggaggagcccctgggccccgacctggaggacctgaagaggcaggtgcagcagcacaaggtgctgcaggaggatctggagcaggaacaggtgagggtgaacagcctgacccacatggtggtggtcgtggacgagagcagcggcgaccatgccaccgccgctctggaggagcaactgaaggtgctgggcgacagatgggccaacatctgcagatggaccgaggacaggtgggtgctgctccaggacatcctcctgaagtggcagaggctgacagaggagcagtgcctgttcagcgcctggctgagcgagaaggaggacgtggtgaacaggatccacaccaccggcttcaaggaccagaacgagatgctcagcagcctgcaaaagctggccgtcctgaaggccgacttggagaaaaagaagcagagcatggacaagctgtacagcctgaagcaggacctcctgagcaccctgaagaacaagagtgtgacccagaagatggaggcgtggctggagaacttcgccaggtgctgggacaacctggtgcagaagctggagaagagcaccgcccagattagccaggccgtgaccaccacccagcctagcctgacccagaccacggtgatggagactgtgaccatggtgaccaccagggagcagatcctggtgaagcacgcccaggaggaactccccccccccccgccccagaagaagagacagatcaccgtggactccgagatcaggaagaggctcgacgtggacatcaccgaactgcatagctggatcaccagaagcgaggccgtgctgcagagccccgagttcgcgatctacaggaaggagggcaacttcagcgacctgaaggaaaaggtgaacgccatcgagagggagaaggccgagaagttcaggaagctccaggacgccagcaggagcgcccaggtgctggtggagcagatggtgaacgagggggtgaacgccgacagcattaggcaggccagtgagcagctgaatagcaggtggatcgagttctgccagctgctcagcgagaggctcaactggctggagtaccagaacaacattatcaccttctacaaccagctgcagcagctggagcagatgaccaccaccgccgagaactggctgaagatccagcccaccacccccagcgagcccaccgccatcaagagtcagctgaagatctgcaaggacgaggtgaacagactgagcgccctgcagccccagatcgagagactcaagatccagtccatcaccctgaaggagaagggccagggccccatgttcctggacgccgacttcgtggccttcaccaaccacttcagccaggtgttcagcgacatgcaggccagggagaaggagctgcagaccatcttcgacaccctgccccccatgaggtaccaagaaaccatgagcgccatcaggacctgggtgcagcagagcgagactaagctgagcatcccccagctgagcgtgaccgactacgagatcatggagcagagactgggcgagctgcaggtgctgcaaagcagcctgcaggagcaacagagcggcctgaactacctgagcaccaccgttaaggagatgagcaagaaggcccccagcgaaatcagcaggaagtaccagctggagttcgaggaaatcgagacaaggtggaagaagctctccagccagctggccgagcactgccagaagttggaggagcaaatgaacaagctgagaaaaatccagaaccacatccagaccctgaagaagtggatggccgaggtggacgtgttcctcaaggaggagtggcccgccctgggcgactccgagattctcaaaaagcagctgaagcagtgtaggctgctggtgaacgacatccagaccatccagcccagcctgaacagcgtgaacgagggcggccagaagatcaagaacgaggccgagcccgagttcgccctgaggctggagatcgagctgaaggagctgaacacccagtgggaccacatgtgccagcaggtgtacgccaggaaggaggccctgaagggcggcctggaaaagaccgtgagcctgcagaaggacctcagcgagatgcacgagtggatgacccaggccgaggaggagtacctggagagggacttcgagtacaagacccccgacgaactgcagaaggccgtggaagagatgaagagggccaaggaggaggcccaacaaaaggaggccaaggtgaagctgctgacagagagcgtgaatagcgtgatcgcccaggcccctcccgtggcccaggaggcgctgaagaaggaactggaaaccctgaccaccaactaccagtggctgtgcaccaggctgaacggcaagtgcaagaccctggaggaggtgtgggcctgctggcacgagctgctgagctacctggaaaaggccaacaagtggctgaacgaggtggagttcaagctgaaaacgaccgagaacatccccggcggcgccgaggagatcagcgaggtgctgcacagcctggagaacctgatgcagcacagcgaggataaccccaaccagatcaggatcctggcccagaccctgaccgacggcggcgtgatggacgaactcatcaacgaggagctggaaaccttcaactccagatggagggagctgcacgaggaggccgtgaggagacagaagctgctcgaacagagcatccagagcgcccaggagatcgaaaagagcctgcacctgatccaggagagcctgaccttcatcgacaagcagctgaccgcctacatcgccgacaaggtggacgccgcccagatgccccaggaggcccagaagatccagagcgacctgaccagccacgagatcagcctggaggagatgaagaagcacaaccagggcaaggaggccgcccagagggtgctgtcccagatcgacgtggcccagaagaagctgcaggacgtgagcatgaagttccggctgttccaaaagcccgcgaacttcgaacagaggctgcaggagagcaagatgatcctggacgaggtcaagatgcacctgcccgccctggaaaccaagagtgtggagcaggaggtggtgcagagccagctcaaccactgcgtgaacctgtacaagagcctgagcgaggtgaagagtgaggtcgagatggtgattaagaccgggaggcagatcgtgcagaaaaagcagaccgagaaccccaaagagctggacgagagggtgaccgccctgaaactgcactacaacgagctgggcgccaaggtgaccgagaggaagcagcagcttgagaagtgcctgaagctgtccaggaagatgaggaaggagatgaacgtgctgaccgagtggctggccgccaccgacatggagctgaccaagaggagcgccgtggaggggatgcccagcaacctggacagcgaggtggcctggggcaaggccacccagaaggagatcgagaagcagaaggtgcacctgaagagtatcaccgaggtgggggaggccctcaagaccgtgctgggcaagaaggagacactggtggaggataagctgagcctcctgaacagcaactggatcgccgtgaccagcagggccgaggagtggctgaacctgctgctggagtatcaaaagcacatggagacattcgaccagaacgtggaccacatcaccaagtggatcatccaggccgacaccctgctcgacgagagcgaaaagaagaagagccagcagaaggaggatgtgttgaagaggctgaaggccgagctgaacgacatcaggcccaaggtggacagcaccagggaccaggccgccaacctgatggccaacaggggcgaccactgcaggaagctggtggagccccagatcagcgagctgaaccacaggttcgccgccatcagccacaggatcaagaccggcaaggccagtatccccctcaaggagctggagcagttcaacagcgacatccagaagctgctggagcctctggaggccgagatccagcagggcgtgaacctgaaggaggaggatttcaacaaggacatgaacgaggataacgagggcaccgtgaaggagttgctccagaggggcgacaacctgcagcagaggatcaccgatgagagaaagagggaggagatcaagatcaagcagcaactgctccagaccaagcacaacgccctgaaggacctgagatcccagaggaggaagaaggccctggagatcagccaccagtggtaccagtacaagcggcaggccgacgacctgctgaagtgcctggacgacatcgagaagaagctggccagcctgcccgagcccagggacgagagaaagattaaggagatcgacagggagctgcagaagaagaaggaggagctgaacgccgtgaggaggcaggccgagggcctgagcgaagatggcgccgccatggccgtggagcccacccagatccagctgtccaagaggtggagggagatcgagagcaagttcgcccaattcaggaggctgaacttcgcccagatccacaccgtgagggaggaaaccatgatggtgatgaccgaggatatgcccctggaaatcagctacgtgcccagcacctacctgaccgagatcacccacgtgagccaggccctgagcgaagtggagcagctgctgaacgcccccgacctgtgcgccaaggacttcgaggatctgtttaagcaggaggagagcctcaagaacatcaaggacatcctgcagcaaagctccggcaggatcgacatcatccacagcaagaaaaccgccgccctgcagagcgccacccccgtggagagagtcaagctgcaggaagccctgagccagatcgacttccagtgggagaaggtcaacaagatgtacaaggacagacagggcaggttcgacagaagcgtggagaagtggaggaggttccactacgacatcaagatcttcaaccagtggctgaccgaagccgagcagttcctgaggaaaacccagatccccgagaactgggagcacgccaagtacaagtggtacctgaaggaactccaggatggcatcggccagaggcagaccgtggtgaggaccctgaatgccaccggcgaggagatcatccagcaaagcagcaagaccgacgccagcatcctgcaggagaaactcggcagcctgaacctgaggtggcaggaggtgtgcaagcagctgagcgagaggaagaagaggctggaggagcagaagaacatcctgagcgagttccagagggacctgaacgagttcgtgctgtggctggaggaggccgacaacatcgcctccatccccctggagcccggcaacgagcaacagctgaaagagaagctggagcaggtcaagctgctggtggaggagctgcccctgaggcagggcatcctgaagcagctgaacgaaaccggcggccccgtgctggtgagcgcccccatcagccccgaggagcaggacaagctggagaacaagctgaagagaaccaacctgcagtggatcaaggtgagcagggccctgcccgagaagcaaggcgagatagaggcccagatcaaggacctgggccaactggaaaagaagctggaggatctcgaagaacagctgaaccacctgctgctgtggctgagccccatcaggaaccagctggagatctataaccagcccaaccaggagggccccttcgacatcaaggagacagagatcgccgtgcaggccaagcagcccgacgtggagggcatcctgtccaagggccagaggctgtacaaggagaagcccgccacacagcccgtgaagagaaagctggaagatctcagcagcgagtggaaaaccgtgaaccatctgctgcaggagctgagggccaagcaacccgacctggcccccggcctgaccaccgtgggcgctagccccagccagaccgtgaccctggtgacccagcccgtggtgaccaaggaaaccgccatcagcaagctggagatgcccagcagcctgatgctggaggtgcctgccctggccgacttcaacagggcctggaccgagctgacagagtggctgagcctgctgggcagggtgatcaagagccagagggtgatggtgggcgacctggaagatatcaacgagatgatcatcaagcagaaggccaccgtgcaggacctggagcagagaaggccccagctggaggagctgatcaccgccgcccagaacctgaagaataagaccagcaaccaggaggccaggacaatcatcaccgacagaatcgagaggatccagaaccagtgggacgaagtgcaggagcacctgcagaacaggaggcagcagctcaacgagatgctgaaggacagcacccagtggctggaagccaaggaggaagctgagcaggtgctgggccaggccagggccaagctggagagctggaaggaaggcccctacaccatcgacgccatccagaagaagatcaccgaaaccaagcagctggccaaggacctgaggcagtggcagatcaacgtggacgtggccaacgacctggccctgaagctgctgcgggactacagcgccgacgacaccaggaaggtgcacatgatcaccgaaaacatcaacgccagctgggggagcatccacaagagggtgagcgagcaggaggccgcgctggaggaaacccacaggctgctgcagcagttccccctggacctcgaaaagttcctggcctggctgaccgaggccgagacaaccgccaacgtgctgcaggacgctaccaggaaggagaggctgctggaggatagcaagggcgtgaaggaactcatgaagcagtggcaggacctgcagggcgagattgaggcccacaccgacgtgtaccacaacctggacgagaacagccagaaggtcctgaggagcttggagggcagcgacgacgccgtgctgctgcagagaaggctggacaacatgaacttcaagtggagcgagctgaggaagaagtccctgaacattagaagccacctggaggctagtagcgaccagtggaaaaggctgcacctgagcctccaggagctgctcgtgtggctgcagctgaaggacgacgagctgagcagacagacccccatcggcggcgacttccccgccgtgcagaagcagaacgacgtgcacagggctttcaagagggagctgaaaaccaaggagcccgtgatcatgagcaccctggagacagtgaggatcttcctcaccgagcagcccctggagggcctggagaagctgtaccaggagcccagggagctgccccccgaggagagagcgcagaacgtgaccagactgctgaggaagcaggccgaggaagtgaacaccgagtgggagaagctgaacctgcacagcaccgactggcagaggaagatcgacgaaaccctggagaggctgcaagagctgcaggaggccatggacgagctggacctgaaactgagacaggccgaggtgatcaagggcagctggcagcccgtcggcgacctgctgatcgacagcctccaggaccacctggaaaaagtgaaggccctgaggggcgagatcgcccctctgaaggagaacgtgagccacgtgaacgacctcgccagacagctgaccaccctgggcatccagctgagcccctacaacctcagcaccctggaagatctgaacaccaggtggaagctgctgcaggtggccgtcgaggacagggtgaggcagctccacgaggcccacagggacttcggccccgccagccagcacttcctgagcaccagcgtgcagggcccctgggagagagccatcagcccgaacaaggtgccctactacatcaaccacgaaacccagaccacctgctgggaccaccccaagatgaccgagctgtaccagagcctggccgacctgaacaatgtgagattctccgcctacagaaccgccatgaagctgaggaggctccagaaggccctgtgcctggacctgctgagcctgagcgccgcctgcgacgccctggaccagcacaacctgaagcaaaacgaccagcccatggacatcctccagatcatcaactgcctgaccaccatctacgacagactggagcaggagcacaacaacctggtgaacgtgcccctgtgcgtggacatgtgcctgaactggctgctgaacgtgtacgacaccggcaggaccggcagaatcagagtgctgagcttcaagaccgggatcatcagcctgtgcaaggcccacctggaggacaagtacaggtacctgttcaagcaggtcagcagcagcaccggcttctgcgaccagagacggctgggcctgctgctgcacgacagcatccagatccccagacagctgggcgaggtggccagcttcggcggcagcaacatcgagcccagcgtgagaagctgcttccagttcgccaacaacaagcccgagatcgaggccgccctgttcctggactggatgaggctggagccccaaagcatggtgtggctgcccgtgctgcacagggtggccgccgccgagacagccaagcaccaggccaagtgcaacatctgcaaggagtgccccatcatcggcttcaggtacaggagcctgaagcacttcaactacgacatctgccagagctgcttcttcagcggcagggtggccaagggccacaagatgcactaccccatggtggagtactgcacccccaccaccagcggcgaggatgtgagagacttcgccaaggtgctgaaaaacaagttcaggaccaagaggtacttcgccaagcaccccagaatgggctacctgcccgtgcagaccgtcctggagggcgacaacatggagacacccgtgaccctgatcaacttctggcccgtggacagcgcccccgccagcagccctcagctgagccacgacgacacccacagcaggatcgagcactacgccagcaggctggccgagatggagaacagcaacggcagctacctgaacgacagcatcagccccaacgagagcatcgacgacgagcacctgctgatccagcactactgccagagcctgaaccaggacagccccctgagccagcccaggtcccccgcccagatcctgatcagcctcgaaagcgaggagaggggcgagctggagagaatcctggccgatctggaggaggagaacaggaacctgcaggccgagtacgacaggctgaagcagcagcacgagcacaagggcctgagccccctgcccagcccccccgagatgatgcccaccagcccccagagccccagggacgccgagctgatcgccgaggcgaagctgctgaggcagcacaagggcaggctggaggccaggatgcagatcctggaggaccacaacaagcagctggagagccagctgcacagactgaggcagctgcttgagcagccccaggccgaagccaaggtgaacggcaccaccgtcagcagccccagcaccagcctgcagaggagcgacagcagccagcccatgctgctgagggtggtgggcagccagaccagcgacagcatgggcgaggaggatgtgctgagccccccccaggacaccagcaccggcctggaggaagtgatggagcagctgaacaacagcttccccagcagcaggggcaggaacacccccggcaagcccatgagggaggacaccatggattacaaggatgacgatgacaagtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggaccgggcgcgccgtttaaactggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggcgcgccgaagagcg

SEQ ID NO: 34 - DNA, 12112 bp, synthetic construct
  source 1..12112: mol_type = other DNA; note = MHCK7-hBGi-DMD-4--
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataa

SEQ ID NO: 35 - DNA, 12500 bp, synthetic construct
  source 1..12500: mol_type = other DNA; note = MHCK7-hBGi-DMD-4-WPRE3-
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtgg

SEQ ID NO: 36 - DNA, 12726 bp, synthetic construct
  source 1..12726: mol_type = other DNA; note = MHCK7-hBGi-DMD-4-WPRE3-PolyA
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcactctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatgctttggtgggaagaagtcgaggactgctacgagcgcgaggacgtgcagaagaaaaccttcaccaaatgggtcaacgcccagttcagcaagttcggcaagcagcacatcgagaacctgttcagcgacctgcaggatggcagaaggctgctggatctgctggaaggcctgacaggacagaagctgcccaaagagaagggcagcacaagagtgcacgccctgaacaacgtgaacaaggccctgagagtgctgcagaacaacaacgtggacctggtcaacatcggcagcaccgacatcgtggacggcaaccacaaactgaccctgggcctgatctggaacatcatcctgcactggcaagtgaagaacgtgatgaagaacatcatggccggcctgcagcagaccaacagcgagaagattctgctgagctgggtccgacagagcacccggaattaccctcaagtgaacgtgatcaacttcaccacctcttggagcgacggactggccctgaatgccctgatccacagccacagacctgacctgttcgactggaacagcgtcgtgtgtcagcagagcgccacacagaggctggaacacgccttcaatatcgccagataccagctgggcatcgagaaactgctggaccccgaggatgtggacaccacctatcctgacaagaaatccatcctcatgtacatcaccagcctgttccaggtgctgccccagcaggtttccatcgaggccattcaagaggtcgagatgctgcccagacctcctaaagtgaccaaagaggaacacttccagctgcaccaccagatgcactactctcagcagatcaccgtgtctctggcccagggctacgagagaacaagcagccccaagcctcggttcaagagctacgcctatacacaggccgcctacgtgaccaccagcgatcctacaagaagcccatttcctagccagcatctggaagcccctgaggacaagagctttggcagcagcctgatggaaagcgaagtgaacctggaccgctaccagacagccctggaagaggttctgagctggctgctgtctgccgaggatacactgcaggctcagggcgagatcagcaacgacgtggaagtggtcaaggaccagtttcacacccacgagggctacatgatggacctgacagcccaccagggcagagtgggcaatattctgcagctgggctccaagctgatcggcacaggcaagctgagcgaggacgaagagacagaggtgcaagagcagatgaacctgctgaacagcagatgggagtgtctgagagtggccagcatggaaaagcagagcaacctgcaccgggtgctgatggatctccagaaccagaagctgaaagagctgaacgactggctgaccaagaccgaggaacggacccggaagatggaagaggaacctctgggacccgacctggaagatctgaaaagacaggtgcagcagcataaggtgctgcaagaggacctcgaacaagagcaagtgcgcgtgaacagcctgacacacatggtggtggtcgtggatgagagcagcggagatcatgccacagccgctctggaagaacagctgaaggtgctgggagacagatgggccaatatctgccggtggaccgaggatagatgggtgctgctccaggacatcctgctgaagtggcagcggctgacagaggaacagtgcctgtttagcgcctggctgtccgagaaagaggacgccgtcaacaagatccacaccaccggcttcaaggatcagaatgagatgctgagcagcctgcagaaactggccgtgctgaaggctgacctggaaaagaaaaagcagtccatgggcaagctgtactccctgaagcaggacctgctgagcacactgaagaacaagtctgtgacccagaaaaccgaggcctggctggacaacttcgcccggtgttgggataacctggtgcagaagctggaaaagtccaccgctcagatctctcaggccgtgaccacaacacagccttctctgacccagaccaccgtgatggaaacagtgaccacagtgacaacccgcgagcagatcctggtcaagcacgcccaagaagaactgcctcctccacctcctcagaagaaacggcagatcacagtggacagcgagatccggaagagactggacgtggacatcaccgagctgcacagctggatcaccagatccgaagccgtgctgcagtcccctgagttcgccatcttcagaaaagagggcaacttctccgacctgaaagagaaagtcaacgccatcgagagagagaaggccgagaagttccggaagctgcaggacgcctctagatctgcccaggctctggtggaacagatggtcaacgaaggcgtgaacgccgacagcatcaagcaggcctcagagcagctgaactcccggtggatcgagttctgtcagctcctgagcgagagactgaactggctggaataccagaacaatatcattgccttctacaaccagctccagcagctcgaacagatgaccaccacagccgagaattggctgaagatccagcctaccacacctagcgagcccaccgccattaagagccagctgaaaatctgcaaggacgaagtgaatcggctgagcgatctgcagccccagatcgaaagactgaaaatccagtctatcgccctcaaagagaaaggacagggccccatgttcctggacgccgattttgtggcctttaccaaccacttcaaacaggtgttctccgacgtgcaggcccgggaaaaagagctgcagaccatcttcgacaccctgcctccaatgagataccaagagacaatgagcgccatccggacctgggtgcagcaaagcgagacaaagctgagcatcccacagctgagcgtgaccgactacgagatcatggaacagagactgggcgaactgcaggccctccagtctagcctgcaagaacagcagtccggcctgtactacctgagcacaaccgtgaaagagatgagcaagaaggcccctagcgagatctcccggaagtaccagagcgagttcgaagagatcgaaggccggtggaagaagctgtctagccagctggttgagcactgccagaaactcgaagaacaaatgaacaagctgcgcaagatccagaatcacatccagacgctgaagaaatggatggccgaggtggacgtgttcctgaaagaagagtggcccgctctgggcgactccgagattctgaagaaacagctcaaacagtgccggctgctggtgtccgatatccagacaatccagccaagcctgaactccgtgaatgaaggcggccagaagatcaagaacgaggccgagcctgagtttgccagcagactggaaaccgaactgaaagaactcaacacccagtgggaccacatgtgccagcaagtgtacgcccggaaagaggccctgaaaggcggactcgaaaagactgtgtctctgcagaaagacctgtctgagatgcacgagtggatgacccaggccgaagaggaatacctggaacgggacttcgagtacaagacccctgatgagctgcaaaaagccgtcgaggaaatgaagcgggccaaagaagaggcccagcagaaagaagccaaagtcaagctgctgaccgagtccgtgaatagcgttatcgctcaggcccctcctgtggctcaagaggctctcaagaaagaactggaaactctgaccaccaactaccagtggctgtgcaccagactgaacggcaagtgcaagacactggaagaagtgtgggcctgctggcacgaactgctgtcctatctggaaaaggccaacaagtggctgaacgaggtggaattcaagctgaaaaccaccgagaacatccctggcggcgctgaagagattagcgaggtgctggacagcctggaaaacctgatgagacacagcgaggacaaccccaatcagatcagaatcctggctcagaccctgaccgatggcggcgtgatggacgagctgatcaacgaggaactcgaaaccttcaacagccggtggcgggaactgcacgaggaagctgttcggaggcagaagttgctggaacagtctatccagagcgcacaagaaaccgagaagtccctgcacctgatccaagagagcctgaccttcatcgacaagcagctggccgcttatatcgccgacaaggtggacgctgctcagatgccccaagaggcacagaagattcagagcgacctgaccagccacgagattagcctggaagaaatgaagaagcacaaccagggcaaagaggccgctcagagagtcctgagccagatcgatgtggcacagaaaaagctccaggatgtgtccatgaagtttcggctgtttcagaagcccgccaacttcgagcagagactgcaagagtccaagatgatcctggacgaagtcaaaatgcatctgcccgcactggaaacaaagtccgtggaacaagaagtggtgcagtcccagctgaaccactgcgtgaacctgtacaagagcctgtccgaagtgaagtctgaggtggaaatggtcatcaagaccggcaggcagatcgtccagaagaagcagaccgagaatcccaaagaactcgacgagagagtgaccgctctgaagctgcactacaatgagctgggcgccaaagtgaccgagcggaagcaacagcttgagaagtgcctgaaactgtctcggaagatgcggaaagaaatgaacgtgctgacagagtggctggccgccaccgatatggaactgactaagagaagcgccgtggaaggcatgcccagcaacctggatagtgaagtggcctggggcaaagccactcagaaagagattgagaagcagaaggtccacctgaagtccatcaccgaagtgggcgaagccctgaaaaccgtgctgggaaagaaagagacactggtcgaggacaagctgtccctgctgaattccaactggatcgccgtgacctccagagctgaggaatggctgaatctgctgcttgagtaccagaaacacatggaaacgttcgaccagaacgtcgaccacatcacaaagtggatcatccaggctgataccctgctggacgagtccgagaagaagaaaccccaacaaaaagaagatgtgctgaagcggctgaaagccgagctgaatgacatccggcctaaggtggacagcaccagagatcaggcagccaacctgatggccaacagaggcgaccactgtcggaagctcgtggaacctcagatcagcgagcttaaccacagatttgccgccatcagccaccggatcaagacaggcaaggccagcattcctctcaaagagcttgagcagttcaacagcgacatccaaaagctgctcgaacccctggaagccgagatccaacagggcgtcaacctcaaagaagaagatttcaacaaggacatgaacgaggacaatgagggcaccgtcaaagaactgctgcagcggggcgataatctgcagcagcggattaccgatgagcgcaagcgggaagagatcaagattaagcagcagctgctgcagaccaagcacaacgccctgaaggacctgcggagccagagaagaaagaaggccctggaaatcagccaccagtggtatcagtacaagcggcaggccgacgacctgctgaagtgcctggatgacatcgagaagaagctggcctctctgcccgagcctagggacgagagaaagatcaaagagatcgaccgcgagctgcagaaaaagaaagaggaactgaacgccgtccgcagacaggccgaaggactttctgaagatggcgccgctatggccgtggaacccacacagattcagctgagcaagcggtggcgggaaatcgagagcaagttcgcccagttccggcggctgaatttcgcccagatccacaccgtgcgggaagagacaatgatggtcatgacagaggacatgccccttgagatcagctacgtgcccagcacctacctgaccgagatcacccatgtgtctcaggccctgctggaagtggaacagctgctgaatgcccctgacctgtgcgccaaggacttcgaggacctgttcaagcaagaggaaagcctgaagaacatcaaggacagcctgcagcagagcagcggccggatcgatatcatccacagcaagaaaaccgccgcactgcagagcgccacacctgtggaaagagtgaagctgcaagaggccctgagccagctggatttccagtgggagaaagtgaacaagatgtacaaggaccggcagggcagattcgaccgcagcgttgaaaagtggcggcggttccactacgacatcaagatcttcaaccagtggctgaccgaggccgagcagttcctgagaaagacacagatccccgagaactgggagcacgccaagtacaagtggtatctgaaagagctgcaggacggcatcggccagagacagacagtcgtgcggacactgaatgccaccggcgaggaaatcatccagcagtccagcaagaccgacgccagcatcctgcaagagaagctgggcagcctgaacctgcggtggcaagaagtgtgcaagcagctgagcgaccggaagaagcggctggaagaacagaagaatatcctgagcgagttccagcgggacctgaacgagttcgtgctgtggctcgaagaggccgacaatatcgccagcattcccctggaacctggcaaagagcagcagctgaaagaaaagctggaacaagtgaaactgctggtcgaggaactgcccctgagacagggaatcctgaaacagctgaacgagacaggcggccctgtgctggtgtctgcccctatttctcccgaggaacaggacaagctggaaaacaagctgaagcagaccaacctgcagtggatcaaggtgtccagagcactgcctgagaagcagggcgagatcgaggcccagatcaaggacctgggacagctcgaaaagaagcttgaggacctcgaagaacagctcaaccatctgctgctttggctgagccccatccggaaccagctggaaatctacaaccagcctaatcaagagggccccttcgacgtgaaagaaaccgagattgccgtgcaggccaagcagcccgatgtggaagagatcctgtctaagggccagcacctgtacaaagagaagcccgccacacagcccgtgaagcggaaactggaagatctgagcagcgagtggaaggccgtgaaccggctgctgcaagaactgagagccaaacagcctgatctggcccctggcctgacaacaattggcgcctctcctacacagaccgtgacactggtcacacagcctgtggtcaccaaagagacagccatcagcaaactggaaatgcccagcagtctgatgctcgaagtgcccgcactggccgacttcaatagagcctggaccgagctgaccgactggctcagtctgctggaccaagtcatcaagtcccagagagtgatggtcggagatcttgaggacatcaacgagatgatcattaagcagaaagccaccatgcaggaccttgagcagagaaggccccagcttgaagaactgatcaccgccgctcagaacctgaaaaacaagaccagcaatcaagaagcccggaccatcatcaccgaccggatcgagagaatccagaaccagtgggacgaagtgcaagagcatctgcagaacagacggcagcagctcaatgagatgctgaaggacagcacacagtggctggaagccaaagaagaagccgagcaggttctcggacaggccagagctaagctggaatcttggaaagagggaccctacaccgtcgacgccatccagaagaagatcaccgagacaaagcagctggccaaggatctgagacagtggcagaccaatgtggacgtggccaacgacctggctctgaagctgctgagagactacagcgccgacgacaccagaaaggtgcacatgatcaccgaaaacatcaacgcctcttggcggagcatccacaagcgggtgtcagagagagaagccgctctggaagaaacacatagactgctccagcagttcccactcgacctggaaaagttcctggcctggctgacagaagccgaaaccaccgctaacgtgctccaggatgccaccagaaaagagcggctgttggaggacagcaagggcgtcaaagaactcatgaagcagtggcaggatctgcaaggggagattgaagcccacaccgacgtgtaccacaacctggacgagaacagccagaagatcctgcggtccctggaaggctctgatgatgctgtgctgcttcagaggcggctggacaacatgaacttcaagtggagcgagctgcggaagaagtccctgaacatcagaagccacctggaagcctccagcgaccagtggaaaagactgcacctgtctctccaagagctgctcgtgtggctgcagctgaaggatgacgagctgagtagacaggcccctatcggcggagattttcccgccgtgcagaaacagaacgacgtgcaccgggccttcaagagagagctgaaaacaaaagaacccgtcatcatgagcaccctggaaaccgtgcggatctttctgaccgagcagcctctggaaggactggaaaagctgtaccaagagcctagagagctgcctcctgaagaaagggcccagaacgtgacacggctgcttagaaagcaggccgaggaagtgaacaccgaatgggagaagctgaacctccactccgccgactggcagcggaagatcgatgagacactggaacggctgcgggaactgcaagaagccacagacgagctggacctgaaactgcggcaggctgaagtgatcaaaggctcctggcagccagtgggcgatctgctgatcgatagcctgcaggaccatctggaaaaagtcaaggccctgcggggagagatcgcccctctcaaagaaaacgtgtcccacgtgaacgatctggccaggcagctgacaacactgggcatccagctgagcccttacaacctgtctaccttggaggacctgaatacccggtggaagctgctccaagtggccgtcgaggatagagtgcggcagctgcatgaggctcacagagattttggacccgccagccagcacttcctgagcacatctgttcaaggcccctgggagagagctatcagccctaacaaggtgccctactacatcaaccacgagacacagaccacctgttgggatcaccccaagatgaccgaactgtatcagtccctggccgacctgaacaacgtgcggtttagcgcctaccggaccgccatgaagctgcgcagactgcagaaagctctgtgcctggacctgctgtctctgagcgctgcttgtgatgccctggaccagcataacctcaagcagaacgaccagcctatggacatcctgcagatcatcaactgcctgaccaccatctacgaccggctcgaacaagagcacaacaacctggtcaacgtgcccctgtgcgtggacatgtgcctgaattggctgctgaacgtgtacgacaccggcagaaccggcaggatcagagtgctgagcttcaagaccggcatcatctccctgtgcaaggcccaccttgaggataagtaccgctacctgtttaagcaggtcgccagcagcaccggcttttgcgaccaaagaaggctgggactgctgctccacgacagcattcagatccctagacagctgggcgaagtggcctctttcggcggctctaatatcgagcctagcgtgcggagctgcttccagttcgccaacaacaagcccgagattgaggccgcactgttcctggactggatgagactggaaccccagagcatggtctggctgcctgtgctgcatagagtggccgcagccgaaacagccaagcaccaggccaagtgcaacatctgcaaagagtgccccatcatcggcttccggtacagatccctgaagcacttcaactacgatatctgccagtcctgcttcttcagcggcagagtggccaagggccacaagatgcactaccccatggtggaatactgcacccctaccacctctggcgaggacgtgcgggattttgccaaggtgctcaagaacaagttccggaccaagcgctacttcgctaagcaccccagaatgggctacctgccagtgcagacagtgcttgagggcgacaacatggaaacccctgtgactctgatcaacttctggcccgtggatagcgcccctgctagttctcctcagctgtctcacgacgatacccacagcagaatcgagcactacgcctccagactggccgagatggaaaacagcaacggcagctacctgaatgacagcatcagccccaacgagagcatcgacgacgaacatctgctcattcagcactactgccagagcctgaatcaggacagccctctgtctcagcctagaagccctgcacagatcctgatcagcctggaaagcgaggaacgcggcgagctggaaagaatcctggcagacctggaagaggaaaaccggaacctgcaggccgaatacgacagactcaagcagcagcacgagcacaagggactgtctccactgccttctccacctgaaatgatgcccacctctccacagagccccagagatgccgagctgattgccgaagccaaactgctgaggcagcacaaaggcagactcgaagccagaatgcagattctcgaagatcacaacaagcagctcgaatcccagctgcacagacttaggcagctgctggaacagcctcaggcagaggccaaagtgaatggcaccaccgtgtctagccctagcacaagcctgcagagatccgacagcagccagccaatgcttctgagagtcgtgggctcccagaccagcgattctatgggcgaagaggacctgctcagccctcctcaggatacaagcaccggcctggaagaagtgatggaacaactgaacaacagcttccccagcagcagaggcagaaacacccctggcaagcccatgcgcgaggacaccatgtgataactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 37 - DNA, 868 bp, synthetic construct; identical to SEQ ID NO: 18
  source 1..868: mol_type = other DNA; note = CMV enhancer-CBA Promoter-AAT Exon-hBGI-hBG Exon

SEQ ID NO: 38 - DNA, 672 bp, synthetic construct
  source 1..672: mol_type = other DNA; note = CMV enhancer-CBA Promoter-AAT Exon
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatc

SEQ ID NO: 39 - DNA, 370 bp, synthetic construct
  source 1..370: mol_type = other DNA; note = CBA Promoter-AAT Exon
tggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatc

SEQ ID NO: 40 - DNA, 566 bp, synthetic construct
  source 1..566: mol_type = other DNA; note = CBA Promoter-AAT Exon-hBGI-hBG Exon
tggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtctctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 41 - DNA, 1652 bp, synthetic construct
  source 1..1652: mol_type = other DNA; note = CAG
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgc

SEQ ID NO: 42 - DNA, 5405 bp, synthetic construct
  source 1..5405: mol_type = other DNA; note = NP-CAG-EGFP-P2A-Fluc-WPRE3-DTS
gtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtacctcgcgaatgcatctagatccgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaacgcgcgatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaatcgcgctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacacgtggcgccatcggatcccgggtggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatct

SEQ ID NO: 43 - DNA, 4594 bp, synthetic construct; identical to SEQ ID NO: 29
  source 1..4594: mol_type = other DNA; note = NP-CBA-hBGi-EGFP-P2A-Fluc-WPRE3-DTS

SEQ ID NO: 44 - DNA, 1477 bp, synthetic construct
  source 1..1477: mol_type = other DNA; note = hNPHS1-hBGi
cacctgaggtcaggagttcgagaccagcgtggccaacatgatgaaaccccgtctctagtaaaaatacaaaaattagccaggcatggtgctatatacctgtagcaccagctacttgggagacagaggtgggagaattacttgaacctgggaggttcaagccatgggaggtggaagttgcagtgagccgagatgccactgcactccagcctgagcaacagagcaagactatctcaagaaaagaaagaaagaaagaaagagacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccctgtctctaaaaaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtgtctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 45 - DNA, 5171 bp, synthetic construct
  source 1..5171: mol_type = other DNA; note = NP-hNPHS1-hBGi-EGFP-P2A-Fluc-WPRE3-DTS
gccacctgaggtcaggagttcgagaccagcgtggccaacatgatgaaaccccgtctctagtaaaaatacaaaaattagccaggcatggtgctatatacctgtagcaccagctacttgggagacagaggtgggagaattacttgaacctgggaggttcaagccatgggaggtggaagttgcagtgagccgagatgccactgcactccagcctgagcaacagagcaagactatctcaagaaaagaaagaaagaaagaaagagacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccctgtctctaaaaaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtgtctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaatcgcgctactaacttcagcctgctgaagcaggctggagacgtggaggagaaccctggacctatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggaagatgtctactgagctgtgcgatccctgctggggactttccgctggggactttccgctggggactttccgccttcagctaaggaagctaccaatatttagaggtacattttgttctagaacaaaatgtaccggtacattttgttctggtacattttgttctattaaggacaacgtaagtatagcgcatagacactggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcatta

SEQ ID NO: 46 - DNA, 1051 bp, synthetic construct; identical to SEQ ID NO: 4
  source 1..1051: mol_type = other DNA; note = MHCK Promoter

SEQ ID NO: 47 - DNA, 4594 bp, synthetic construct; identical to SEQ ID NO: 29
  source 1..4594: mol_type = other DNA; note = NP-CBA-hBGi-EGFP-P2A-Fluc-WPRE3-DTS

SEQ ID NO: 48 - DNA, 4786 bp, synthetic construct; identical to SEQ ID NO: 30
  source 1..4786: mol_type = other DNA; note = NP-MHCK7-hBGi-EGFP-P2A-Fluc-WPRE3-DTS

SEQ ID NO: 49 - DNA, 15459 bp, synthetic construct; identical to SEQ ID NO: 31
  source 1..15459: mol_type = other DNA; note = pUC57-CBA-hBGi-DMD-4-WPRE3-DTS

SEQ ID NO: 50 - DNA, 15634 bp, synthetic construct; identical to SEQ ID NO: 32
  source 1..15634: mol_type = other DNA; note = pUC57-MHCK7-hBGi-DMD-4-WPRE3-DTS

SEQ ID NO: 51 - DNA, 281 bp, synthetic construct
  source 1..281: mol_type = other DNA
tctggatccactgcttaaatacggacgaggacagggccctgtctcctcagcttcaggcaccaccactgacctgggacagtgaatcgtaagtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc